(19) United States
(12) Patent Application Publication
(10) Pub. No.: US 2002/0155119 A1
(43) Pub. Date: Oct. 24, 2002

(54) Isolation and use of fetal urogenital sinus expressed sequences

(76) Inventors: Robert A. Sikes, Gordonsville, VA (US); Leland W.K. Chung, Lovingston, VA (US); Jin Hee Kim, Santa Monica, CA (US); Claudia Fasciana, Rotterdam (NL); Jan Trapman, Mijnsheerenland (NL)

Correspondence Address: PENNIE & EDMONDS LLP, 1667 K STREET NW, SUITE 1000, WASHINGTON, DC, 20006

(21) Appl. No.: 09/933,797

(22) Filed: Aug. 22, 2001

Related U.S. Application Data
(63) Continuation of application No. 09/482,933, filed on Jan. 14, 2000, now abandoned.
Application No. 09/482,933 is a continuation of application No. PCT/US99/10746, filed on May 14, 1999.
(60) Provisional application No. 60/085,383, filed on May 14, 1998.

(51) Int. Cl. C12Q 1/68; C07H 21/04; A61K 39/00; C12P 21/02; C12N 5/06; C07K 14/435

(52) U.S. Cl. 424/185.1; 536/23.5; 435/325; 435/320.1; 435/69.1; 530/350; 435/6

(57) ABSTRACT

The invention comprises methods for identifying biomarkers useful for prognostic or diagnostic assays of human prostate disease, and for identifying those fetal genes which are differentially expressed between prostate cancers versus normal or benign prostate.

### CROSS-REFERENCE TO RELATED APPLICATIONS

This application claims the benefit under 35 U.S.C. section 119(e) of co-pending U.S. provisional application Ser. No. 60/085,383, filed May 14, 1998, the entire text of which is herein incorporated by reference without disclaimer.

### STATEMENT AS TO RIGHTS TO INVENTIONS MADE UNDER FEDERALLY-SPONSORED RESEARCH AND DEVELOPMENT

Part of the work performed during development of this invention utilized U.S. Government funds. The U.S. Government has certain rights in this invention. This work was supported by National Institutes of Health Grant PHFDK47596.

### I. FIELD OF THE INVENTION

The present invention relates to the study of normal and diseased prostate development. More particularly, the present invention relates to methods and compositions relates to novel nucleotide sequences which can be used for the diagnosis, prognosis and treatment of prostatitis and benign and malignant growth of the prostate gland. More particularly, the present invention concerns probes and methods useful in diagnosing, identifying and monitoring the progression of diseases of the prostate through measurements of fetal gene products.

### II. BACKGROUND OF THE INVENTION

Prostatic Hyperplasia

Development of prostatic hyperplasia is an almost universal phenomenon in aging men. The prostate weighs only a few grams at birth; at puberty it undergoes androgen-mediated growth and reaches the adult size of about 20 g by age 20. It remains stable in size for about 25 years, and during the fifth decade a second growth spurt commences in the majority of men. Consequently, the disorder affects men over the age of 45 and increases in frequency with age so that by the eighth decade more than 90 percent of men have prostatic hyperplasia at autopsy. Since the development of BPH is not a major cause of death, the development of effective therapies has been slow despite BPH been a leading cause of morbidity in elderly men. The prostate surrounds the urethra, and prostatic hyperplasia is the most common cause of obstruction to urinary outflow in men. The disorder occurs in all populations but may be less common in the Orient. The mean age for development of symptomatic disease is about 65 years for whites and about 60 years for blacks. At present, it is not clear whether prostatic hyperplasia predisposes the prostate to the development of prostatic cancer (Harrison's Principles of Internal Medicine, Chapter 97, p 596, 14th Edition, McGraw Hill, 1999).

Unlike the pubertal growth spurt which involves the gland diffusely, prostatic hyperplasia begins in the periurethral region as a localized proliferation and progresses to compress the remaining normal gland. Histologically, the hyperplastic tissue is nodular and composed of varying amounts of glandular epithelium, stroma, and smooth muscle. The hyperplasia can compress and obstruct the urethra; the hyperplastic gland can also grow posteriorly to obstruct the rectum and cause constipation.

At present, the pathogenesis is not well understood, but two necessary features for the process are aging and the presence of testes; whether the testes play a direct or permissive role is not known, but the active androgen that mediates prostatic growth at all ages is dihydrotestosterone, which is formed within the prostate from plasma testosterone (Harrison's Principles of Internal Medicine, Chapter 97, pp 597, 14th Edition, McGraw Hill, 1999).

Prostatic Carcinoma

Cancer of the prostate is the most common malignancy in men in the United States and the third most common cause of cancer death in men above age 55 (after carcinomas of the lung and colon). In the United States there are approximately 317,000 newly diagnosed cases and more than 41,000 deaths from the disorder each year. Only about a third of cases identified at autopsy are manifest clinically. The disease is rare before age 50, and the incidence increases with age. The frequency varies in different parts of the world. The United States has 14 deaths per 100,000 mean per year, compared with 22 for Sweden and 2 for Japan. However, Japanese immigrants to the United States develop prostatic cancer at a frequency similar to other men in this country, suggesting that environmental factors are the principal cause for population differences. The disease is more common among American blacks than whites; the reason for this difference is not known. Some carcinomas of the prostate are slow-growing and may persist for long periods without causing significant symptoms, whereas others behave aggressively. It is not known whether tumors can become more malignant with time (Harrison's Principles of Internal Medicine, Chapter 97, p 598, 14th Edition, McGraw Hill, 1999).

Prostatitis

The term prostatitis has been used for various inflammatory conditions affecting the prostate, including acute and chronic infections with specific bacteria and, more commonly, instances in which signs and symptoms of prostatic inflammation are present but no specific organisms can be detected. Patients with acute bacterial prostatitis can usually be identified on the basis of typical symptoms and signs, pyuria, and bacteriuria. To classify a patient with suspected chronic prostatitis correctly, first-void and midstream urine specimens, a prostatic expressate, and a postmassage urine specimen should be quantitatively cultured and evaluated for numbers of leukocytes. On the basis of the results of these studies, patients can be classified as having chronic bacterial prostatitis, chronic nonbacterial prostatitis, or prostatodynia. Patients with suspected chronic prostatitis usually have low back pain, perineal or testicular discomfort, mild dysuria, and lower urinary obstructive symptoms. Microscopic pyuria may be the only objective manifestation of prostatic disease (Harrison's Principles of Internal Medicine, Chapter 131, pp823, 14th Edition, McGraw Hill, 1999).

Carcinoma of the prostate (PCA) is the second-most frequent cause of cancer related death in men in the United States (Boring, 1993). The increased incidence of prostate cancer during the last decade has established prostate cancer as the most prevalent of all cancers (Carter and Coffey, 1990). Although prostate cancer is the most common cancer found in United States men, (approximately 200,000 newly diagnosed cases/year), the molecular changes underlying its genesis and progression remain poorly understood (Boring et al., 1993). According to American Cancer Society estimates, the number of deaths from PCA is increasing in excess of 8% annually.

An unusual challenge presented by prostate cancer is that most prostate tumors do not represent life threatening conditions. Evidence from autopsies indicate that 11 million American men have prostate cancer (Dbom, 1983). These figures are consistent with prostate carcinoma having a protracted natural history in which relatively few tumors progress to clinical significance during the lifetime of the patient. If the cancer is well-differentiated, organ-confined and focal when detected, treatment does not extend the life expectancy of older patients.

Unfortunately, the relatively few prostate carcinomas that are progressive in nature are likely to have already metastasized by the time of clinical detection. Survival rates for individuals with metastatic prostate cancer are quite low. Between these two extremes are patients with prostate tumors that will metastasize but have not yet done so. For these patients, surgical removal of their prostates is curative and extends their life expectancy. Therefore, determination of which group a newly diagnosed patient falls within is critical in determining optimal treatment and patient survival.

Although clinical and pathologic stage and histological grading systems (e.g., Gleason's) have been used to indicate prognosis for groups of patients based on the degree of tumor differentiation or the type of glandular pattern (Carter and Coffey, 1989; Diamond et al., 1982), these systems do not predict the progression rate of the cancer. While the use of computer-system image analysis of histologic sections of primary lesions for “nuclear roundness” has been suggested as an aide in the management of individual patients (Diamond et al., 1982), this method is of limited use in studying the progression of the disease.

It is known that the processes of transformation and tumor progression are associated with changes in the levels of messenger RNA species (Slamon et al., 1984; Sager et al., 1993; Mok et al., 1994; Watson et al., 1994). Recently, a variation on PCR analysis known as RNA fingerprinting has been used to identify messages differentially expressed in ovarian or breast carcinomas (Liang et al., 1992; Sager et al., 1993; Mok et al., 1994; Watson et al., 1994). By using arbitrary primers to generate “fingerprints” from total cell RNA, followed by separation of the amplified fragments by high resolution gel electrophoresis, it is possible to identify RNA species that are either up-regulated or down-regulated in cancer cells. Results of these studies indicated the presence of several markers of potential utility for diagnosis of breast or ovarian cancer, including a6-integrin (Sager et al., 1993), DEST001 and DEST002 (Watson et al., 1994), and LF4.0 (Mok et al., 1994).

There are two unique features of prostate cancer not shared by most of the other forms of human malignancies. First, the prevalence of prostate cancer is extremely high. In 1998 there are estimated to be 184,500 new cases diagnosed in American men accounting for nearly one-third of all male cancers (Parker et al., Cancer Journal for Clinicians. 47:5-27, 1997). At the same time there are predicted to be 39,000 deaths from prostate cancer or about 21% of the number of new cases. Prostate cancer is a disease of advancing years. By the sixth decade of life the chances of having prostate cancer are 1 in 5. In this group of men prostate cancer is the second most common form of death by cancer. But this is still only a fraction of those diagnosed. In contrast, the prevalence/incidence of lung cancer virtually equals the mortality from lung cancer with approximately 90,000 cases diagnosed and 90,000 deaths expected (Parker et al., Cancer Journal for Clinicians. 47:5-27, 1997; Boring et al., Cancer Journal for Clinicians. 44:7-26, 1994) and has remained unchanged for several years. The significant disparity between the total number of men diagnosed with prostate cancer and those dying from the disease emphasizes the importance of developing molecular markers to differentiate the virulent from indolent forms of prostate cancer and to help stratify management options for men presenting with prostate tumors. Current staging and prognostic modalities for human prostate cancer are woefully inadequate. Furthermore, our comprehension of the genetic influence over prostate carcinogenesis is lacking, although several genetic and epigenetic factors have been identified that correlate with the development of a more aggressive neoplastic phenotype (Bostwick et al., Journal of Cellular Biochemistry—Supplement. 19:283-289, 1994; Bostwick et al., Journal of Cellular Biochemistry, Supplement. 19:197-201, 1994; Rinker-Schaeffer et al., Cancer & Metastasis Reviews. 12:3-10,1993; Thompson et al., Genomics. 13:402-8,1992; Zhau et al., Journal of Cellular Biochemistry, Supplement. 19:208-216, 1994; Veltri et al., Journal of Cellular Biochemistry, Supplement. 19:249-258,1994). These include proliferation markers, pathophysiologic markers, growth factor-growth factor receptors, oncogenes, tumor suppressor genes, neuroendocrine products, and the extracellular matrix. These have been used either alone or in combination as prognostic and diagnostic markers. Unfortunately, these are poor markers and, to date, no single factor has been identified that can accurately predict the malignant potential of any given prostate tumor nor predict which patient with localized disease will eventually relapse or progress (Bostwick et al., Journal of Cellular Biochemistry—Supplement. 19: 283-289,1994;Veltri et al., Journal of Cellular Biochemistry, Supplement. 19: 249-258, 1994).

A second unique feature of prostate cancer is that it responds poorly to chemotherapy. Men with prostate cancer may initially respond well to hormonal or radiation therapy, but inevitably will relapse. Once an androgen independent phenotype is acquired, no effective therapies are currently available. For this reason, it is critically important to develop both novel management strategies and therapeutic modalities for the treatment of advanced prostate disease. One obstacle to studying human prostate cancer has been the long latency period, generally ≧25-35 years, that is required for the progression of prostate cancer from its latent morphologic forms to clinically-apparent disease. To overcome this long latency period, our laboratory has developed a human prostate cancer progression model utilizing the LNCaP cell line, a useful androgen responsive cell line as the starting material from which were generated an array of cell-lineage related sublines. This model has been shown to be relevant to human prostate cancer progression and mimics the pathophysiologic changes observed clinically as a tumor acquires increasingly metastatic and tumorigenic characteristics (Thalmann et al., Cancer Research. 54:2577-2581,1994; Wu et al., The International Journal of Cancer. Submitted October 1997.:, 1997; Wu et al., International Journal of Cancer. 57:406-12, 1994).

Recent studies have identified several recurring genetic changes in prostate cancer including, inter alia: allelic loss (particularly loss of chromosome 8p and 16q) (Bova, et al., 1993; Macoska et al., 1994; Carter et al., 1990); generalized DNA hypermethylation (Isaacs et al., 1994); point mutations or deletions of the retinoblastoma (Rb) and p53 genes (Bookstein et al., 1990a; Bookstein et al., 1990b; Isaacs et al., 1991); alterations in the level of certain cell-cell adhesion molecules (i.e., E-cadherin/alpha-catenin) (Carter et al., 1990; Morton et al., 1993; Umbas et al., 1992) and aneuploidy and aneusomy of chromosomes detected by fluorescence in situ hybridization (FISH), particularly chromosomes 7 and 8 Macoska et al., 1994; Visakorpi et al., 1994; Takahashi et al., 1994; Alcaraz et al., 1994).

The analysis of DNA content/ploidy using flow cytometry and FISH has been demonstrated to have utility predicting prostate cancer aggressiveness (Pearsons et al., 1993; Macoska et al., 1994; Visakorpi et al., 1994; Takahashi et al., 1994; Alcaraz et al., 1994; Pearsons et al., 1993), but these methods are expensive, time-consuming, and the latter methodology requires the construction of centromere-specific probes for analysis. Additionally, specific nuclear matrix proteins have been reported to be associated with prostate cancer. (Partin et al., 1993). However, these protein markers apparently do not distinguish between benign prostate hyperplasia and prostate cancer. Martin et al., 1993). Unfortunately, markers which cannot distinguish between benign and malignant prostate tumors are deemed to be of little value to urologists.

From the clinical perspective, successfully managing a prostate cancer patient is often a difficult task for the practicing urologist. Although clinicians examine tumor architecture, measure prostate-specific antigen (PSA) levels, and estimate tumor volume to help guide clinical decision-making, these currently available staging and prognostic modalities are insufficient. Studies performed on other types of cancers, such as testicular, liver and colon, have determined that these tumors can express gene products that are normally expressed only in the fetus during normal development of those organs. Some examples of these fetal proteins, also called oncofetal markers, include alpha fetoprotein (AFP) and carcinoembryonic antigen (CEA). For testicular, liver, and colon tumors, AFP and CEA are commonly used in diagnosis, therapy, and for predicting and monitoring responses to treatment. Unfortunately, these particular markers are not applicable to the management of prostate cancer and, to date, no similar oncofetal gene(s) have been identified with any prognostic or diagnostic potential for prostate disease. It has been demonstrated that embryonic or fetal genes, such as the carcinoembryonic antigen (CEA) and alpha-fetoprotein (AFP), are frequently re-expressed in a spatially or temporally inappropriate manner during carcinogenesis. This aberrant expression has particular importance for tumor biology and therapy. Both CEA and AFP have provided significant contribution to the detection and management of germ cell, gastrointestinal and hepatobiliary cancers. Although this approach has demonstrated successful application to the diagnosis and treatment of the aforementioned tumors, no such correlates to these markers have been developed for prostate cancer.

As a result, there remain, however, deficiencies in the prior art with respect to the identification of the fetal genes linked with the progression of prostate cancer and the development of diagnostic methods to monitor disease progression. Likewise, the identification of fetal genes which are differentially expressed in prostate cancer would be of considerable importance in the development of a rapid, inexpensive method to diagnose prostate cancer. The present invention addresses the deficiencies in the prior art.

### III. SUMMARY OF THE INVENTION

One aspect of the present invention is novel isolated nucleic acid segments that are useful as described herein as hybridization probes and primers that specifically hybridize to prostate disease markers. These disease markers, including both known genes and previously undescribed genes, are described herein as those fetal genes shown to be differentially expressed (either up- or down-regulated) in a prostate disease state as compared to a normal prostate. The novel isolated nucleic acid segments are designated herein as ug92, ug93, ug96, ug101, ug102, ug106, ug120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194. The invention further comprises an isolated nucleic acid of between about 14 and about 100 bases in length, either identical to or complementary to a portion of the same length occurring within the disclosed sequences.

The present invention comprises proteins and peptides with amino acid sequences encoded by the aforementioned isolated nucleic acid segments. The invention also comprises methods for identifying biomarkers useful for prognostic or diagnostic assays of human prostate disease, and for identifying those fetal genes which are differentially expressed between prostate cancers versus normal or benign prostate.

The invention further comprises methods for detecting prostate cancer cells in biological samples, using hybridization primers and probes designed to specifically hybridize to prostate cancer markers. The hybridization probes are identified and designated herein as ug092, ug093,ug096,ug101, ug102, ug106, ug120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194. This method further comprises measuring the amounts of nucleic acid amplification products formed when primers selected from the designated sequences are used.

The invention further comprises the prognosis and/or diagnosis of prostate cancer by measuring the amounts of nucleic acid amplification products formed as above. The invention comprises methods of treating individuals with prostate cancer by providing effective amounts of substances, including, inter alia, antibodies and/or antisense DNA molecules which bind to the products of the above mentioned isolated nucleic acids. The invention further comprises kits for performing the above-mentioned procedures, containing amplification primers and/or hybridization probes.

The present invention further comprises production of antibodies specific for proteins or peptides encoded by ug092, ug093, ug096, ug101, ug102, ug106, ug 120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194, and the use of those antibodies for diagnostic applications in detecting diseases of the prostate, including, without limitation, prostatitis, and benign and malignant growth of the prostate gland.

The invention further comprises therapeutic treatment of diseases of the prostate, including, without limitation, prostatitis, and benign and malignant growth of the prostate gland by administration of pharmaceutically effective doses of inhibitors specific for proteins encoded by the aforementioned markers.

The invention further comprises therapeutic treatment of diseases of the prostate, including, without limitation, prostatitis, and benign and malignant growth of the prostate gland by the use of novel isolated nucleic acid segments comprising ug092, ug093, ug096, ug101, ug102, ug106, ug120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194 for the development of therapeutic modalities including tissue-or cancer-specific gene promoters for use in gene therapy by naked DNA delivery or viral toxic gene therapy, growth suppression of prostate cancer by replacement gene therapy, and tissue specific gene products used to develop immunotherapeutic agents using peptide specific anti-prostate cancer vaccines or adoptive immunotherapies using peptide/protein specific cytotoxic T-cells.

### IV. BRIEF DESCRIPTION OF THE DRAWINGS

The following drawings form part of the present specification and are included to further demonstrate certain aspects of the present invention. The invention may be understood better by reference to one or more of these drawings in combination with the detailed description of specific embodiments presented herein.

FIG. 1: Nucleotide sequences for 787 urogenital sinus (UGS)-derived ESTs

FIG. 2: Representative grid for columns “E”. The dot E1 is the result of pooled clones 297, 306, 314, 323, 333, 342, 352 and 360. Dot E16 on the matrix represents the addition of clones 297, 298, 299, 300, 301, 302, 304 and 305.

FIG. 3: Duplicate Dot Matrix Array filters spotted with 320 cDNA clones in pooled sets of 64 as depicted in FIG. 2 for set E. Each pair of columns A-E represents a new set of 64 clones in overlapping arrays. Radiolabeled cDNAs, reverse transcribed from LNCaP and C4-2 human prostate cancer cell line RNAs, were used as probes. The arrows indicate the pair of spots corresponding to UG311 as depicted in FIG. 2 that are lost with progression from LNCaP to C4-2.

FIG. 4: Northern blot analysis using the UG311 EST as a probe on a progression series of lineage-related prostate cancer cell lines either not-treated or treated for 48 h with 1 nM R1881 (androgen).

FIG. 5: Fold luciferase induction in LNCaP and C4-2 prostate cancer cell lines. Cells that are stably transfected with pTET-on were assayed by transient transfection to determine their ability to induce luciferase expression from pTRE-luc in response to doxycycline.

FIG. 6: RNA bolts using 30 μg total RNA from the cell lines as indicated. LNCaP through C4-2B#4 represent lineage-related cell lines having progressively more androgen independence and metastatic capacity. The +/− signs signify whether or not the samples were treated with 1 nM R1881 for 48 hours in serum-free conditions.

FIG. 7: Schematic representation of UGS-derived cDNA protein coding sequence into bacterial expression vector pGEX-4T for generating recombinant protein for use as immunogen.

FIG. 8: Urogenital sinus cDNA clone summary obtained from GelView Contig run: A determination of the range of independent sequences.

FIG. 9: Additional consensus sequence of differentially expressed cDNA clones.

### V. DETAILED DESCRIPTION OF THE INVENTION

Current staging and prognostic modalities for human prostate cancer are woefully inadequate. Furthermore, the current comprehension of the genetic influence over prostate carcinogenesis is lacking, although several genetic and epigenetic factors have been identified that correlate with the development of a more aggressive neoplastic phenotype. In the human, mesenchymal-epithelial interaction maintains the functional integrity of the adult prostate gland. Prior investigations in our laboratory have demonstrated that fetal mesenchyme has the capacity to initiate glandular overgrowth of the adult rodent prostate (Sikes et al., Biology of Reproduction. 43: 353-62, 1990; Chung et al., Biology of Reproduction. 31:155-163, 1984), reduce anaplasia in the Dunning prostatic adenocarcinoma model (Chung et al., Prostate. 17:165-74, 1990; Hayashi et al., Cancer Research. 50: 4747-54, 1990), and induce the differentiation of androgen receptor deficient urogenital sinus epithelium (UGE) into functional prostate tissue (Chung et al., Biology of Reproduction. 31:155-163, 1984; Chung et al., Prostate. 17:165-74, 1990; Hayashi et al., Cancer Research. 50:4747-54, 1990; Chung et al., Molecular Biology Reports. 23:13-19, 1996. Prostatic carcinogenesis may be explained by aberrant instructive influences derived from its underlying stroma, as the microenvironment surrounding cancer epithelium has been demonstrated to determine tumor growth and malignant potential (Bissell et al., The Journal of Theoretical Biology. 99:31-68, 1982; Jacobson, Science. 152:25-34, 1966).

Consequently, it is believed that abnormal prostate growth and prostate carcinogenesis may result from abnormalities of the constituents of the stromal-epithehal milieu. The inductive role of stroma has been demonstrated in a wide variety of glandular tissues during embryonic development, including the prostate (Sakakura et al., Developmental Biology. 72:201-210, 1979; Drews et al., Cell. 10:401-404, 1977; Franks et al., The Journal of Pathology. 100:113-120, 1970; McNeal, Investigative Urology. 15:340-5,1978; Cunha et al., Journal of Steroid Biochemistry. 14:1317-24, 1981; Cunha et al., Biology of Reproduction. 22:19-42, 1980). Prostatic proliferation in the adult may result from a reawakening of dormant embryonic growth elements present in the prostatic stroma (Chung et al., Prostate. 4:503-11, 1983). It has been demonstrated that fetal urogenital sinus mesenchyme (UGM), a fetal form of prostatic stroma, is inductive and can redirect prostatic epithelial growth and differentiation (Chung et al., Biology of Reproduction. 31:155-163, 1984; Cunha et al., Endocrine Reviews. 8:338-62, 1987). Marked growth and expression of tissue-specific secretory proteins can be induced when fetal UGM is recombined with either fetal or adult prostate epithelium (Gleave et al., Cancer Research. 51:3753-61, 1991; Chung, Cancer Surveys. 23:33-42, 1995) or when it is implanted directly into the adult prostate gland (Evans, The Brittish Journal of Cancer. 68: 1051-1060,1993; Sokoloff et al., Cancer. 77:1862-1872,1996). Implanted fetal mesenchyme can induce differentiation and growth of adult rat urogenital cells (Chung et al., Prostate. 17:165-74, 1990; Hayashi et al., Cancer Research. 50:4747-54, 1990). Recombinants of androgen receptor deficient fetal mesenchyme with either fetal or adult epithelium failed to produce appropriate cytodifferentiation when recombined with fetal UGM lacking the androgen receptor (derived from testicular feminization, Tfm/y, fetuses)(Chung et al., Biology of Reproduction. 31:155-163, 1984; Chung, Cancer Surveys. 23:33-42, 1995). This further supports the contention that paracrine mediators between stroma and epithelium are prerequisite for prostate growth and morphogenesis.

Inductive influences from stroma to prostatic epithelial differentiation can be classified as either directive or permissive, depending upon the sources of embryonic epithelium and the age of both the inductive and responsive fetal tissue (Han et al., Carcinogenesis. 16:951-954, 1995). Thereafter, the ultimate growth potential of the embryonic and adult prostatic epithelium in tissue recombinants or in situ will be dictated by the presence of inductive stroma. By varying the amount of embryonic stroma used in the construction of tissue recombinants (Chung, Cancer Surveys. 23:33-42, 1995) or by inserting fetal UGM directly into the adult prostate (Evans, The Brittish Journal of Cancer. 68:1051-1060, 1993), the growth potential of prostatic epithelium is dictated entirely by the amount of UGM present in either tissue recombinants or in the induced chimeric adult gland. Hence, mesenchymal agents can induce normal and neoplastic prostate growth and differentiation. Furthermore, prostate carcinogenesis mimics a reversion to a more developmentally primitive state. Therefore, the differential expression of prostate-embryonic genes may direct neoplastic transformation or, at least, identify when a clonal population has undergone such transformation.

The temporal involvement of steroid hormones and growth factors is paramount to prostate development. Prostate growth and differentiation is tightly regulated by androgens and is influenced by a number of soluble peptide growth factors and their receptors (Cunha et al., Recent Progress in Hormone Research. 39:559-98, 1983). A close reciprocal association between stromal and epithelial tissues also has a fundamental role in normal, benign, and malignant prostate development. Mesenchymal and epithelial differentiation depends upon the stimulatory effects of dihydrotestosterone, inductive growth factors and peptides, and embryonic factors (Cunha et al., Recent Progress in Hormone Research. 39:559-98, 1983). The combination of epidermal growth factor, transforming growth factor-β, insulin growth factor, and gonadotropin can induce differentiation of reproductive cells. Other studies have demonstrated that many of the properties associated with tumor progression and metastasis in hormone-refractory prostate cancer cell lines can be altered after treatment with cytokines (Sokoloff et al., Cancer. 77:1862-1872, 1996; Ritchie et al., Endocrinology. 138:1145-1150, 1997). Suppression of prostate cancer cell growth correlated with the downregulation of oncogene, suppressor gene, growth factor, and adhesion molecule gene expression.

Our laboratory studies the interaction of prostate cancer cells and their surrounding environment, known as stroma. It has been shown that the stroma can alter normal prostate behavior and contribute to cancer progression. Furthermore, it has been shown that when normal prostate tissue is exposed to fetal tissue, the growth and development of the normal prostate resembles that of a neoplastic prostate. Many similarities exist between fetal tissue and neoplastic tissue. These include an increased rate of growth, the predilection to invade and migrate to distant locations as well as an inclination for undergoing internal changes that can detour a cell from maturing normally. These cells either remain underdeveloped or acquire the characteristics of a cell with non-prostate qualities or fetal prostate qualities.

In the human, mesenchymal-epithelial interaction maintains the functional integrity of the adult prostate gland. Indeed, some of the prognostic markers discussed previously, such as the extracellular matrix, basement membrane integrity and intermediate filament/integrin alterations, demonstrate that changes in the mesenchymal-epithelial interaction are hallmarks of cancer development. Prior investigations have demonstrated that fetal prostate mesenchyme has the capacity to initiate glandular overgrowth of adult rodent prostates (McKinnell et al., New York: Plenum Press, 1989; Pierce, N.J.: Prentiss-Hall, Inc., 1978; Sikes et al., Biology of Reproduction. 43:353-62, 1990; Chung et al., Biology of Reproduction. 31:155-163, 1984), reduce anaplasia in the Dunning prostatic adenocarcinoma model (Chung et al., Prostate. 17:165-74, 1990; Hayashi et al., Cancer Research. 50:4747-54, 1990), and induce the differentiation of androgen receptor deficient urogenital sinus epithelium into functional prostate tissue (Sikes et al., Biology of Reproduction. 43:353-62, 1990; Chung et al., Molecular Biology Reports.23:13-19,1996; Bissell et al., The Journal of Theoretical Biology. 99:31-68, 1982). As such, the instructive influence of fetal mesenchymal gene products to drive differentiation and growth is of particular interest for cancer biology since fetal tissues: divide rapidly, migrate and invade, remodel and differentiate; all of which are properties fetal tissues have in common with cancer cells. Additionally, many cancers have an embryonic appearance and express fetal (Jacobson, Science. 152:25-34, 1966) gene or differentiated (Sakakura et al., Developmental Biology. 72:201-210,1979) gene products in an inappropriate temporally or spatially manner.

Since there has been no examination of fetal prostate gene expression in prostate cancer, we sought to examine the possibility that UGS-derived gene products might be oncofetal markers for prostate cancer. Therefore, in order to investigate the role of gene expression during prostate embryogenesis and to then relate this to changes in gene expression during prostate cancer progression, a cDNA library was made from murine urogenital sinus (UGS), the prostate progenitor, and 787 clones were generated and randomly screened. Of these 787 cDNA clones, 728 generated useful sequence information. These 728 fetal murine urogenital sinus (UGS)-derived cDNA clones were subsequently screened for their expression in the LNCaP (androgen dependent, non-tumorigenic) and lineage derived C4-2 (androgen-independent, tumorigenic metastatic to bone) cell lines that closely mimic the natural progression of human prostate cancer but in a much shorter time frame. This model allows the comparison of the 728 UGS derived cDNA clones with the expressed genes from a less-aggressive versus a more aggressive prostate cancer model. This screen has identified over 33 UGS expressed sequence tags or cDNA clones whose level of expression changes when the androgen sensitive LNCaP probed filters are compared to the androgen independent C4-2 clone probed filters.

This represents the first documented evidence that fetal urogenital sinus-derived genes have been associated with the malignant potential of prostate cancer. This evidence immediately suggests that fetal prostate gene expression or loss in the prostate is significant in the development and progression of prostate cancer. In addition to clarifying the role of embryonic influences on prostate carcinogenesis, these differentially-expressed genes can also be developed into prognostic markers and targets for gene therapy and other therapeutic modalities to detect and prevent the development and progression of human prostate cancer. Furthermore, such gene products encoded by these genes can also be used to predict a prostate cancer's aggressiveness and to differentiate prostate cancers exhibiting different degrees of virulence. Such an approach has never before been employed with fetal prostate genes and thus represents a novel approach to diagnosis of prostate cancer. The methods employed herein may thus be used to examine those fetal genes which show the greatest change in expression and to develop improved techniques of monitoring patients with prostate cancer and novel therapies to prevent or retard cancerous changes in the prostate. Both of these advances should make a significant impact on the clinical management of men with prostate disease.

The more than 780 randomly screened fetal murine urogenital sinus (UGS)-derived cDNA clones described above have the following designations: ua1a2 (SEQ ID NO:1); ua1a4f (SEQ ID NO:2); ua1a4r (SEQ ID NO:3 ); ua1a6f (SEQ ID NO:4 ); ua1a6r (SEQ ID NO:5 ); ua1b4f (SEQ ID NO:6); ua1b4r (SEQ ID NO:7); ua1b5 (SEQ ID NO:8); ua1d1 (SEQ ID NO:9); ua1c6f (SEQ ID NO:10); ua1c6r (SEQ ID NO:11); ua1c6r (SEQ ID NO:12); ua1d2 (SEQ ID NO:13); ua1d4 (SEQ ID NO:14); ua1e1f (SEQ ID NO:15); ua1e1r (SEQ ID NO: 16); ua1e3f (SEQ ID NO:17); ua1e3r (SEQ ID NO:18); ua1e5r (SEQ ID NO:19); ua1e6f (SEQ ID NO:20); ua1e6r (SEQ ID NO:21); ua1f1r (SEQ ID NO:22); ua1f3f (SEQ ID NO: 23); ua1f3r (SEQ ID NO:24); ua1f4f (SEQ ID NO:25); ua1f5f (SEQ ID NO:26); ua1f6f (SEQ ID NO:27); ua1f6r (SEQ ID NO:28); ua1g2f (SEQ ID NO:29); ua1g4r (SEQ ID NO:30); ua1g5f (SEQ ID NO:31); ua1h2f (SEQ ID NO:32); ua1h3f (SEQ ID NO:33); ua1h4 (SEQ ID NO:34); ua2h6f (SEQ ID NO:35); ua2h6r (SEQ ID NO:36); ua2h6f (SEQ ID NO:37); ua2h6r (SEQ ID NO:38); ua2h7r (SEQ ID NO:39); ug1rcon (SEQ ID NO:40); ug2rcon (SEQ ID NO: 41); ug3 meld (SEQ ID NO:42); ug4rcon (SEQ ID NO:43); ug5rcon (SEQ ID NO:44); ug6rcon (SEQ ID NO:45); ug6?con (SEQ ID NO:46); ug7rcon (SEQ ID NO:47); ug8rcon (SEQ ID NO:48); ug9rcon (SEQ ID NO:49); ug10rcon (SEQ ID NO:50); ug11rcon (SEQ ID NO:51); ug12rcon (SEQ ID NO:52); ug13rcon (SEQ ID NO:53); ug14rcon (SEQ ID NO:54); ug15rcon (SEQ ID NO:55); ug16/38180 (SEQ ID NO:56); ug17rcon (SEQ ID NO:57); ug18reon (SEQ ID NO:58); ug19rcon (SEQ ID NO:59); ug20r2 (SEQ ID NO:60); ug21rcon (SEQ ID NO:61); ug22rcon (SEQ ID NO:62); ug23rcon (SEQ ID NO:63); ug24rcon (SEQ ID NO:64); ug25rcon (SEQ ID NO:65); ug26rcon (SEQ ID NO:66); ug27rcon (SEQ ID NO: 67); ug28rcon (SEQ ID NO:68); ug29rcon (SEQ ID NO:69); ug30rcon (SEQ ID NO:70); ug31con (SEQ ID NO:71); ug32rcon (SEQ ID NO:72); ug33con (SEQ ID NO:73); ug34con (SEQ ID NO:74); ug35con (SEQ ID NO:75); ug36rcon (SEQ ID NO:76); ug37rcon (SEQ ID NO:77); ug39rcon (SEQ ID NO:78); ug42rcon (SEQ ID NO:79); ug43rcon (SEQ ID NO: 80); ug42con (SEQ ID NO:81); ug43rcon (SEQ ID NO:82); ug44rcon (SEQ ID NO:83); ug45 (SEQ ID NO:84); ug46 (SEQ ID NO:85); ug47rcon (SEQ ID NO:86); ug48 (SEQ ID NO: 87); ug49rcon (SEQ ID NO:88); ug50rcon (SEQ ID NO:89); ugs51rcon (SEQ ID NO:90); ug52rcon (SEQ ID NO:91); ug53rcon (SEQ ID NO:92); ug54 (SEQ ID NO:93); ug53rcon (SEQ ID NO:94); ug56 (SEQ ID NO:95); ug 57rcon (SEQ ID NO:96); ug58rcon (SEQ ID NO:97); ug59 (SEQ ID NO:98); ug60 (SEQ ID NO:99); ug61rcon (SEQ ID NO:100); ug62rcon (SEQ ID NO:101); ug63rcon (SEQ ID NO:102); ug64rcon (SEQ ID NO:103); ug65rcon (SEQ ID NO:104); ug66rcon (SEQ ID NO:105); ug67rcon (SEQ ID NO:106); ug68rcon (SEQ ID NO:107); ug69rcon (SEQ ID NO:108); ug70rcon (SEQ ID NO:109); ug71rcon (SEQ ID NO:110); ug72rcon (SEQ ID NO:111); ug73rcon (SEQ ID NO:112); ug74rcon (SEQ ID NO:113); ug75rcon (SEQ ID NO:114); ug76rcon (SEQ ID NO:115); ug77rcon (SEQ ID NO:116); ug78rcon (SEQ ID NO:117); ug79rcon (SEQ ID NO:118); ug81rcon (SEQ ID NO:119); ug82rcon (SEQ ID NO:120); ug83rcon (SEQ ID NO:121); ug84rcon (SEQ ID NO:122); ug85rcon (SEQ ID NO:123); ug86rcon (SEQ ID NO:124); ug87rcon (SEQ ID NO:125); ug88rcon (SEQ ID NO:126); ug89rcon (SEQ ID NO:127); ug90rcon (SEQ ID NO:128); ug91rcon (SEQ ID NO:129); ug92rcon (SEQ ID NO:130); ug93rcon (SEQ ID NO:131); ug94rcon (SEQ ID NO:132); ug95rcon (SEQ ID NO:133); ug96 (SEQ ID NO:134); ug96rcon (SEQ ID NO:135); ug97rcon (SEQ ID NO:136); ug98rcon (SEQ ID NO:137); ug99rcon (SEQ ID NO:138); ug100rcon (SEQ ID NO:139); ug101rcon (SEQ ID NO:140); ug102rcon (SEQ ID NO:141); ug103rcon (SEQ ID NO:142); ug104rcon (SEQ ID NO:143); ug106rcon (SEQ ID NO:144); ug107rcon (SEQ ID NO:145); ug108rcon (SEQ ID NO:146); ug109rcon (SEQ ID NO:147); ug110rcon (SEQ ID NO:148); ug111rcon (SEQ ID NO:149); ug112 (SEQ ID NO:150); ug113rcon (SEQ ID NO:151); ug114rcon (SEQ ID NO:152); ug115rcn1o (SEQ ID NO:153); ug115rcon (SEQ ID NO:154); ug116rcon (SEQ ID NO:155); ug117 (SEQ ID NO:156); ug118 (SEQ ID NO:157); ug119 (SEQ ID NO:158); ug120rcon (SEQ ID NO:159); ug121(SEQ ID NO:160); ug122rcon (SEQ ID NO:161); ug123 (SEQ ID NO:162); ug124 (SEQ ID NO:163); ug125 (SEQ ID NO:164); ug126 (SEQ ID NO:165); ug127 (SEQ ID NO:166); ug128 (SEQ ID NO:167); ug129 (SEQ ID NO:168); ug130 (SEQ ID NO:169); ug130r2 (SEQ ID NO:170);ug131 (SEQ ID NO:171); ug132 (SEQ ID NO:172); ug133 (SEQ ID NO:173); ug134 (SEQ ID NO:174); ug135 (SEQ ID NO:175); ug136rcon (SEQ ID NO:176); ug137rcon (SEQ ID NO:177); ug138 (SEQ ID NO:178); ug139 (SEQ ID NO:179); ug140 (SEQ ID NO:180); ug141rcon (SEQ ID NO:181); ug142 (SEQ ID NO:182); ug143 (SEQ ID NO:183); ug144 (SEQ ID NO:184); ug145 (SEQ ID NO: 185); ug146 (SEQ ID NO:186); ug147 (SEQ ID NO:187); ug148 (SEQ ID NO:188); ug149rcon (SEQ ID NO:189); ug150rcon (SEQ ID NO:190); ug151rcon (SEQ ID NO:191); ug152rcon (SEQ ID NO:192); ug153rcon (SEQ ID NO:193); ug154rcon (SEQ ID NO:194); ug155rcon (SEQ ID NO:195); ug156rcon (SEQ ID NO:196); ug157rcon (SEQ ID NO:197); ug158 (SEQ ID NO:198); ug159 (SEQ ID NO:199); ug160 (SEQ ID NO:200); ug161 (SEQ ID NO:201); ug162 (SEQ ID NO:202); ug163rcon (SEQ ID NO:203); ug164rcon (SEQ ID NO:204); ug165rcon (SEQ ID NO:205); ug166rcon (SEQ ID NO:206); ug167rcon (SEQ ID NO:207); ug168rcon (SEQ ID NO:208); ug169rcon (SEQ ID NO:209); ug170rcon (SEQ ID NO:210); ug171rcon (SEQ ID NO:211); ug172rcon (SEQ ID NO:212); ug173rcon (SEQ ID NO:213); ug174rcon (SEQ ID NO:214); ug175rcon (SEQ ID NO:215); ug176rcon (SEQ ID NO:216); ug177rcon (SEQ ID NO:217); ug178rcon (SEQ ID NO:218); ug179rcon (SEQ ID NO:219); ug180rcon (SEQ ID NO:220); ug181rcon (SEQ ID NO:221); ug182 (SEQ ID NO: 222); ug183rcon (SEQ ID NO:223); ug184rcon (SEQ ID NO:224); ug185 (SEQ ID NO:225); ug185rcon (SEQ ID NO:226); ug186rcon (SEQ ID NO:227); ug187rcon (SEQ ID NO:228); ug188rcon (SEQ ID NO:229); ug189rcon (SEQ ID NO:230); ug190rcon (SEQ ID NO:231); ug191rcon (SEQ ID NO:232); ug192rcon (SEQ ID NO:233); ug193 (SEQ ID NO:234); ug194 (SEQ ID NO:235); ug195 (SEQ ID NO:236); ug196 (SEQ ID NO:237); ug197 (SEQ ID NO:238); ug198 (SEQ ID NO:239); ug199 (SEQ ID NO:240); ug200 (SEQ ID NO:241); ug201 (SEQ ID NO:242); ug202 (SEQ ID NO:243); ug203 (SEQ ID NO:244); ug204 (SEQ ID NO:245); ug205 (SEQ ID NO:246); ug206 (SEQ ID NO:247); ug207 (SEQ ID NO:248); ug208 (SEQ ID NO:249); ug209 (SEQ ID NO:250); ug210 (SEQ ID NO:251); ug210 (SEQ ID NO:252); ug211 (SEQ ID NO:253); ug212 (SEQ ID NO:254); ug213 (SEQ ID NO:255); ug214 (SEQ ID NO:256); ug215 (SEQ ID NO:257); ug216 (SEQ ID NO:258); ug217 (SEQ ID NO:259); ug218 (SEQ ID NO:260); ug219 (SEQ ID NO:261); ug220 (SEQ ID NO:262); ug221 (SEQ ID NO:263); ug222 (SEQ ID NO:264); ug223 (SEQ ID NO:265); ug224 (SEQ ID NO:266); ug225 (SEQ ID NO:267); ug226 (SEQ ID NO:268); ug227 (SEQ ID NO:269); ug228 (SEQ ID NO:270); ug229 (SEQ ID NO:271); ug230 (SEQ ID NO:272); ug231 (SEQ ID NO:273); ug232 (SEQ ID NO:274); ug233 (SEQ ID NO:275); ug234 (SEQ ID NO:276); ug235 (SEQ ID NO:277); ug236 (SEQ ID NO:278); ug237 (SEQ ID NO:279); ug238 (SEQ ID NO:280); ug239 (SEQ ID NO:281); ug240 (SEQ ID NO:282); ug241 (SEQ ID NO:283); ug242 (SEQ ID NO:284); ug243 (SEQ ID NO:285); ug244 (SEQ ID NO:286); ug245 (SEQ ID NO:287); ug246 (SEQ ID NO:288); ug247 (SEQ ID NO:289); ug248 (SEQ ID NO:290); ug249 (SEQ ID NO:291); ug250 (SEQ ID NO:292); ug251 (SEQ ID NO:293); ug252 (SEQ ID NO:294); ug253 (SEQ ID NO:295); ug254 (SEQ ID NO:296); ug255 (SEQ ID NO:297); ug256 (SEQ ID NO:298); ug257 (SEQ ID NO:299); ug258 (SEQ ID NO:300); ug259 (SEQ ID NO:301); ug260 (SEQ ID NO:302); ug261 (SEQ ID NO:303); ug262 (SEQ ID NO:304); ug263 (SEQ ID NO:305); ug264 (SEQ ID NO:306); ug265 (SEQ ID NO:307); ug266 (SEQ ID NO:308); ug267 (SEQ ID NO:309); ug268 (SEQ ID NO:310); ug269 (SEQ ID NO:311); ug270 (SEQ ID NO:312); ug271 (SEQ ID NO:313); ug272 (SEQ ID NO:314); ug273 (SEQ ID NO:315); ug274 (SEQ ID NO:316); ug275 (SEQ ID NO:317); ug276 (SEQ ID NO:318); ug277 (SEQ ID NO:319); ug278 (SEQ ID NO:320); ug279 (SEQ ID NO:321); ug280 (SEQ ID NO:322); ug281 (SEQ ID NO:323); ug282 (SEQ ID NO:324); ug283 (SEQ ID NO:325); ug284 (SEQ ID NO:326); ug285 (SEQ ID NO:327); ug286 (SEQ ID NO:328); ug287 (SEQ ID NO:329); ug288 (SEQ ID NO:330); ug289 (SEQ ID NO:331); ug290 (SEQ ID NO:332); ug291 (SEQ ID NO:333); ug292 (SEQ ID NO:334); ug293 (SEQ ID NO:335); ug294 (SEQ ID NO:336); ug295 (SEQ ID NO:337); ug296 (SEQ ID NO:338); ug297 (SEQ ID NO:339); ug298 (SEQ ID NO:340); ug299 (SEQ ID NO:341); ug300 (SEQ ID NO:342); ug301 (SEQ ID NO:343); ug303 (SEQ ID NO:344); ug304 (SEQ ID NO:345); ug305 (SEQ ID NO:346); ug306 (SEQ ID NO:347); ug307 (SEQ ID NO:348); ug308 (SEQ ID NO:349); ug309 (SEQ ID NO:350); ug310 (SEQ ID NO:351); ug311 (SEQ ID NO:352); ug312 (SEQ ID NO:353); ug313 (SEQ ID NO:354); ug314 (SEQ ID NO:355); ug315 (SEQ ID NO:356); ug316 (SEQ ID NO:357); ug317 (SEQ ID NO:358); ug318 (SEQ ID NO:359); ug320 (SEQ ID NO:360); ug321 (SEQ ID NO:361); ug322 (SEQ ID NO:362); ug323 (SEQ ID NO:363); ug324 (SEQ ID NO:364); ug325 (SEQ ID NO:365); ug326 (SEQ ID NO:366); ug327 (SEQ ID NO:367); ug328 (SEQ ID NO:368); ug329 (SEQ ID NO:369); ug330 (SEQ ID NO:370); ug331 (SEQ ID NO:371); ug332 (SEQ ID NO:372); ug333 (SEQ ID NO:373); ug334 (SEQ ID NO:374); ug335 (SEQ ID NO:375); ug336 (SEQ ID NO:376); ug337 (SEQ ID NO:377); ug338 (SEQ ID NO:378); ug339 (SEQ ID NO:379); ug340 (SEQ ID NO:380); ug341 (SEQ ID NO:381); ug342 (SEQ ID NO:382); ug343 (SEQ ID NO:383); ug344 (SEQ ID NO:384); ug345 (SEQ ID NO:385); ug346 (SEQ ID NO:386); ug347 (SEQ ID NO:387); ug348 (SEQ ID NO:388); ug349 (SEQ ID NO:389); ug350 (SEQ ID NO:390); ug351 (SEQ ID NO:391); ug352 (SEQ ID NO:392); ug353 (SEQ ID NO:393); ug354 (SEQ ID NO:394); ug355 (SEQ ID NO:395); ug356 (SEQ ID NO:396); ug357 (SEQ ID NO:397); ug358 (SEQ ID NO:398); ug359 (SEQ ID NO:399); ug360 (SEQ ID NO:400); ug361 (SEQ ID NO:401); ug362 (SEQ ID NO:402); ug363 (SEQ ID NO:403); ug364 (SEQ ID NO:404); ug365 (SEQ ID NO:405); ug366 (SEQ ID NO:406); ug367 (SEQ ID NO:407); ug368 (SEQ ID NO:408); ug369 (SEQ ID NO:409); ug370 (SEQ ID NO:410); ug371 (SEQ ID NO:411); ug372 (SEQ ID NO:412); ug373 (SEQ ID NO:413); ug374 (SEQ ID NO:414); ug375 (SEQ ID NO:415); ug376 (SEQ ID NO:416); ug377 (SEQ ID NO:417); ug378 (SEQ ID NO:418); ug379 (SEQ ID NO:419); ug380 (SEQ ID NO:420); ug381 (SEQ ID NO:421); ug382 (SEQ ID NO:422); ug383 (SEQ ID NO:423); ug384 (SEQ ID NO:424); ug385 (SEQ ID NO:425); ug386 (SEQ ID NO:426); ug387 (SEQ ID NO:427); ug388 (SEQ ID NO:428); ug389 (SEQ ID NO:429); ug390 (SEQ ID NO:430); ug391 (SEQ ID NO:431); ug392 (SEQ ID NO:432); ug393 (SEQ ID NO:433); ug394 (SEQ ID NO:434); ug395 (SEQ ID NO:435); ug386 (SEQ ID NO:436); ug397 (SEQ ID NO:437); ug398 (SEQ ID NO:438); ug399 (SEQ ID NO:439); ug400 (SEQ ID NO:440); ug401 (SEQ ID NO:441); ug402 (SEQ ID NO:442); ug403 (SEQ ID NO:443); ug404 (SEQ ID NO:444); ug406 (SEQ ID NO:445); ug407 (SEQ ID NO:446); ug408 (SEQ ID NO:447); ug411 (SEQ ID NO:448); ug412 (SEQ ID NO:449); ug413 (SEQ ID NO:450); ug414 (SEQ ID NO:451); ug415 (SEQ ID NO:452); ug416 (SEQ ID NO:453); ug417 (SEQ ID NO:454); ug418 (SEQ ID NO:455); ug420 (SEQ ID NO:456); ug421 (SEQ ID NO:457); ug422 (SEQ ID NO:458); ug423 (SEQ ID NO:459); ug424 (SEQ ID NO:460); ug425 (SEQ ID NO:461); ug426 (SEQ ID NO:462); ug427 (SEQ ID NO:463); ug428 (SEQ ID NO:464); ug429 (SEQ ID NO:465); ug430 (SEQ ID NO:466); ug431 (SEQ ID NO:467); ug432 (SEQ ID NO:468); ug433 (SEQ ID NO:469); ug434 (SEQ ID NO:470); ug435 (SEQ ID NO:471); ug436 (SEQ ID NO:472); ug437 (SEQ ID NO:473); ug439 (SEQ ID NO:474); ug441 (SEQ ID NO:475); ug442 (SEQ ID NO:476); ug443 (SEQ ID NO:477); ug444 (SEQ ID NO:478); ug445 (SEQ ID NO:479); ug446 (SEQ ID NO:480); ug447 (SEQ ID NO:481); ug448 (SEQ ID NO:482); ug449 (SEQ ID NO:483); ug450 (SEQ ID NO:484); ug451 (SEQ ID NO:485); ug452 (SEQ ID NO:486); ug453 (SEQ ID NO:487); ug454 (SEQ ID NO:488); ug455 (SEQ ID NO:489); ug456 (SEQ ID NO:490); ug457 (SEQ ID NO:491); ug458 (SEQ ID NO:492); ug459 (SEQ ID NO:493); ug460 (SEQ ID NO:494); ug461 (SEQ ID NO:495); ug462 (SEQ ID NO:496); ug463 (SEQ ID NO:497); ug464 (SEQ ID NO:498); ug465 (SEQ ID NO:499); ug466 (SEQ ID NO:500); ug467 (SEQ ID NO:501); ug468 (SEQ ID NO:502); ug470 (SEQ ID NO:503); ug471 (SEQ ID NO:504); ug472 (SEQ ID NO:505); ug473 (SEQ ID NO:506); ug474 (SEQ ID NO:507); ug475 (SEQ ID NO:508); ug476 (SEQ ID NO:509); ug477 (SEQ ID NO:510); ug478 (SEQ ID NO:511); ug479 (SEQ ID NO:512); ug480 (SEQ ID NO:513); ug481 (SEQ ID NO:514); ug482 (SEQ ID NO:515); ug483 (SEQ ID NO:516); ug484 (SEQ ID NO:517); ug485 (SEQ ID NO:518); ug486 (SEQ ID NO:519); ug487 (SEQ ID NO:520); ug488 (SEQ ID NO:521); ug489 (SEQ ID NO:522); ug491 (SEQ ID NO:523); ug492 (SEQ ID NO:524); ug493 (SEQ ID NO:525); ug494 (SEQ ID NO:526); ug495 (SEQ ID NO:527); ug496 (SEQ ID NO:528); ug497 (SEQ ID NO:529); ug498 (SEQ ID NO:530); ug499 (SEQ ID NO:531); ug500 (SEQ ID NO:532); ug501 (SEQ ID NO:533); ug502 (SEQ ID NO:534); ug504 (SEQ ID NO:535); ug505 (SEQ ID NO:536); ug506 (SEQ ID NO:537); ug507 (SEQ ID NO:538); ug508 (SEQ ID NO:539); ug509 (SEQ ID NO:540); ug510 (SEQ ID NO:541); ug511 (SEQ ID NO:542); ug514 (SEQ ID NO:543); ug516 (SEQ ID NO:544); ug517 (SEQ ID NO:545); ug518 (SEQ ID NO:546); ug519 (SEQ ID NO:547); ug520 (SEQ ID NO:548); ug521 (SEQ ID NO:549); ug522 (SEQ ID NO:550); ug523 (SEQ ID NO:551); ug524 (SEQ ID NO:552); ug525 (SEQ ID NO:553); ugs001 (SEQ ID NO:554); ugs003 (SEQ ID NO:555); ugs005 (SEQ ID NO: 556); ugs006 (SEQ ID NO:557); ugs007 (SEQ ID NO:558); ugs008 (SEQ ID NO:559); ugs009 (SEQ ID NO:560); ugs010 (SEQ ID NO:561); ugs011 (SEQ ID NO:562); ugs012 (SEQ ID NO:563); ugs013 (SEQ ID NO:564); ugs014 (SEQ ID NO:565); ugs015) (SEQ ID NO:566); ugs016 (SEQ ID NO:567); ugs017 (SEQ ID NO:568); ugs018 (SEQ ID NO:569); ugs019 (SEQ ID NO:570); ugs020 (SEQ ID NO:571); ugs021 (SEQ ID NO:572); ugs022 (SEQ ID NO:573); ugs023 (SEQ ID NO:574); ugs024 (SEQ ID NO:575); ugs025 (SEQ ID NO:576); ugs026 (SEQ ID NO:577); ugs027 (SEQ ID NO:578); ugs028 (SEQ ID NO:579); ugs029 (SEQ ID NO:580); ugs030 (SEQ ID NO:581); ugs031 (SEQ ID NO:582); ugs032 (SEQ ID NO:583); ugs033 (SEQ ID NO:584); ugs034 (SEQ ID NO:585); ugs035 (SEQ ID NO:586); ugs036 (SEQ ID NO:587); ugs038 (SEQ ID NO:588); ugs039 (SEQ ID NO:589); ugs040 (SEQ ID NO:590); ugs041 (SEQ ID NO:591); ugs042 (SEQ ID NO:592); ugs043 (SEQ ID NO:593); ugs044 (SEQ ID NO:594); ugs045 (SEQ ID NO:595); ugs046 (SEQ ID NO:596); ugs047 (SEQ ID NO:597); ugs048 (SEQ ID NO:598); ugs050 (SEQ ID NO:599); ugs051 (SEQ ID NO:600); ugs052 (SEQ ID NO:601); ugs054 (SEQ ID NO:602); ugs055 (SEQ ID NO:603); ugs059 (SEQ ID NO:604); ugs060 (SEQ ID NO:605); ugs063 (SEQ ID NO:606); ugs064 (SEQ ID NO:607); ugs065 (SEQ ID NO:608); ugs066 (SEQ ID NO:609); ugs067 (SEQ ID NO:610); ugs068 (SEQ ID NO:611); ugs070 (SEQ ID NO:612); ugs071 (SEQ ID NO:613); ugs072 (SEQ ID NO:614); ugs074 (SEQ ID NO:615); ugs077 (SEQ ID NO:616); ugs078 (SEQ ID NO:617); ugs080 (SEQ ID NO:618); ugs084 (SEQ ID NO:619); ugs085 (SEQ ID NO:620); ugs086 (SEQ ID NO:621); ugs087 (SEQ ID NO:622); ugs088 (SEQ ID NO:623); ugs090 (SEQ ID NO:624); ugs091 (SEQ ID NO:625); ugs092 (SEQ ID NO:626); ugs093 (SEQ ID NO:627); ugs094 (SEQ ID NO:628); ugs095 (SEQ ID NO:629); ugs099 (SEQ ID NO:630); ugs100 (SEQ ID NO:631); ugs101 (SEQ ID NO:632); ugs102 (SEQ ID NO:633); ugs103 (SEQ ID NO:634); ugs104 (SEQ ID NO:635); ugs105 (SEQ ID NO:636); ugs106 (SEQ ID NO:637); ugs107 (SEQ ID NO:638); ugs108 (SEQ ID NO:639); ugs110 (SEQ ID NO:640); ugs111 (SEQ ID NO:641); ugs112 (SEQ ID NO:642); ugs113 (SEQ ID NO:643); ugs114 (SEQ ID NO:644); ugs115 (SEQ ID NO:645); ugs116 (SEQ ID NO:646); ugs117 (SEQ ID NO:647); ugs118 (SEQ ID NO:648); ugs119 (SEQ ID NO:649); ugs120 (SEQ ID NO:650); ugs121 (SEQ ID NO:651); ugs122 (SEQ ID NO:652); ugs123 (SEQ ID NO:653); ugs125 (SEQ ID NO:654); ugs126 (SEQ ID NO:655); ugs127 (SEQ ID NO:656); ugs128 (SEQ ID NO:657); ugs129 (SEQ ID NO:658); ugs131 (SEQ ID NO:659); ugs133 (SEQ ID NO:660); ugs134 (SEQ ID NO:661); ugs135 (SEQ ID NO:662); ugs136 (SEQ ID NO:663); ugs137 (SEQ ID NO:664); ugs138 (SEQ ID NO:665); ugs139 (SEQ ID NO:666); ugs140 (SEQ ID NO:667); ugs142 (SEQ ID NO:668); ugs143 (SEQ ID NO:669); ugs144 (SEQ ID NO:670); ugs145 (SEQ ID NO:671); ugs146 (SEQ ID NO:672); ugs147 (SEQ ID NO:673); ugs148 (SEQ ID NO:674); ugs149 (SEQ ID NO:675); ugs150 (SEQ ID NO:676); ugs151 (SEQ ID NO:677); ugs152 (SEQ ID NO:678); ugs153 (SEQ ID NO:679); ugs156 (SEQ ID NO:680); ugs157 (SEQ ID NO:681); ugs159 (SEQ ID NO:682); ugs160 (SEQ ID NO:683); ugs161 (SEQ ID NO:684); ugs163 (SEQ ID NO:685); ugs164 (SEQ ID NO:686); ugs165 (SEQ ID NO:687); ugs167 (SEQ ID NO:688); ugs168 (SEQ ID NO:689); ugs172 (SEQ ID NO:690); ugs173 (SEQ ID NO:691); ugs174 (SEQ ID NO:692); ugs175 (SEQ ID NO:693); ugs177 (SEQ ID NO:694); ugs178 (SEQ ID NO:695); ugs179 (SEQ ID NO:696); ugs180 (SEQ ID NO:697); ugs181 (SEQ ID NO:698); ugs182 (SEQ ID NO:699); ugs183 (SEQ ID NO:700); ugs184 (SEQ ID NO:701); ugs186 (SEQ ID NO:702); ugs187 (SEQ ID NO:703); ugs188 (SEQ ID NO:704); ugs190 (SEQ ID NO:705); ugs191 (SEQ ID NO:706); ugs192 (SEQ ID NO:707); ugs193 (SEQ ID NO:708); ugs194 (SEQ ID NO:709); ugs195 (SEQ ID NO:710); ugs196 (SEQ ID NO:711); ugs198 (SEQ ID NO:712); ugs199 (SEQ ID NO:713); ugs200 (SEQ ID NO:714); ugs201 (SEQ ID NO:715); ugs202 (SEQ ID NO:716); ugs203 (SEQ ID NO:717); ugs204 (SEQ ID NO:718); ugs205 (SEQ ID NO:719); ugs206 (SEQ ID NO:720); ugs208 (SEQ ID NO:721); ugs210 (SEQ ID NO:722); ugs211 (SEQ ID NO:723); ugs212 (SEQ ID NO:724); ugs213 (SEQ ID NO:725); ugs214 (SEQ ID NO:726); ugs216 (SEQ ID NO:727); ugs217 (SEQ ID NO:728); ugs218 (SEQ ID NO:729); ugs219 (SEQ ID NO:730); ugs221 (SEQ ID NO:731); ugs223 (SEQ ID NO:732); ugs225 (SEQ ID NO:733); ugs226 (SEQ ID NO:734); ugs227 (SEQ ID NO:735); ugs228 (SEQ ID NO:736); ugs229 (SEQ ID NO:737); ugs231 (SEQ ID NO:738); ugs232 (SEQ ID NO:739); ugs233 (SEQ ID NO:740); ugs234 (SEQ ID NO:741); ugs235 (SEQ ID NO:742); and ugs236 (SEQ ID NO:743).

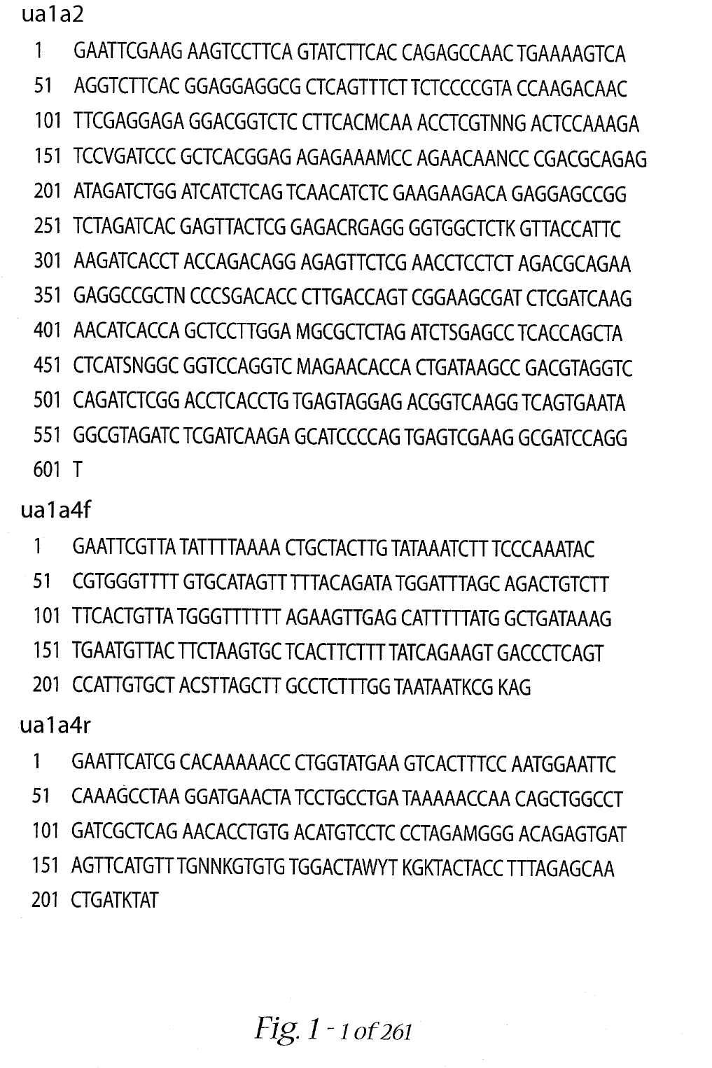

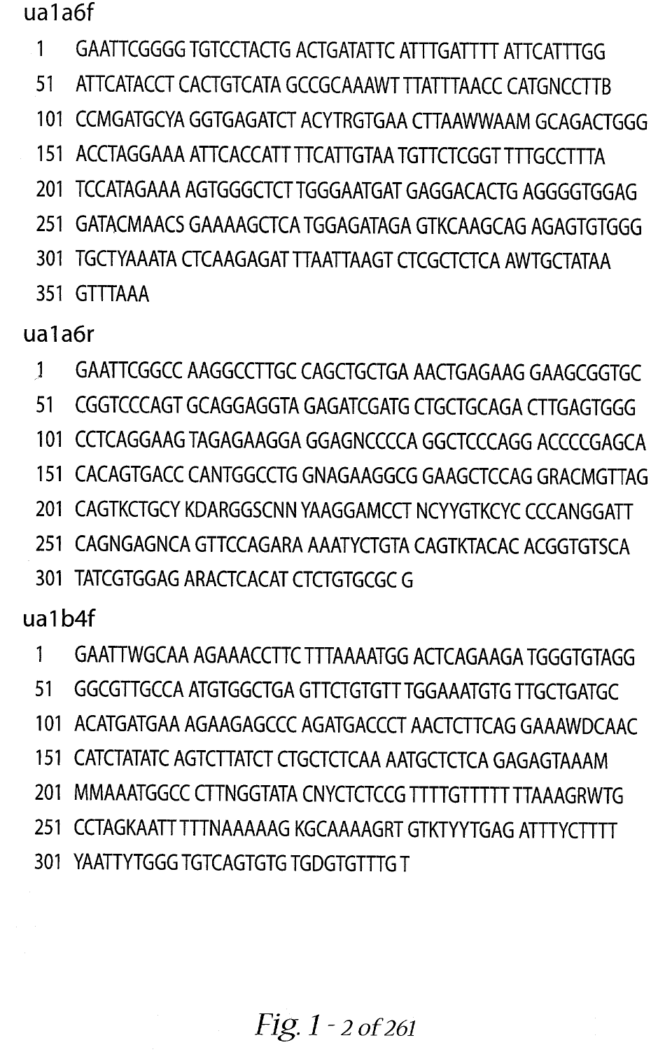

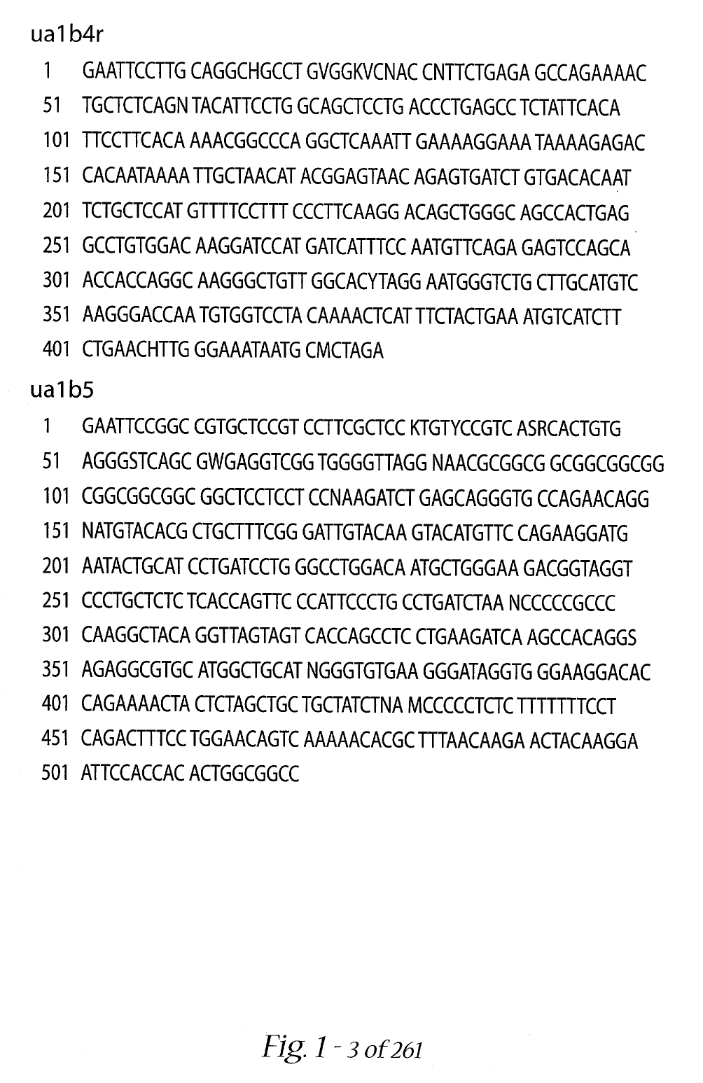

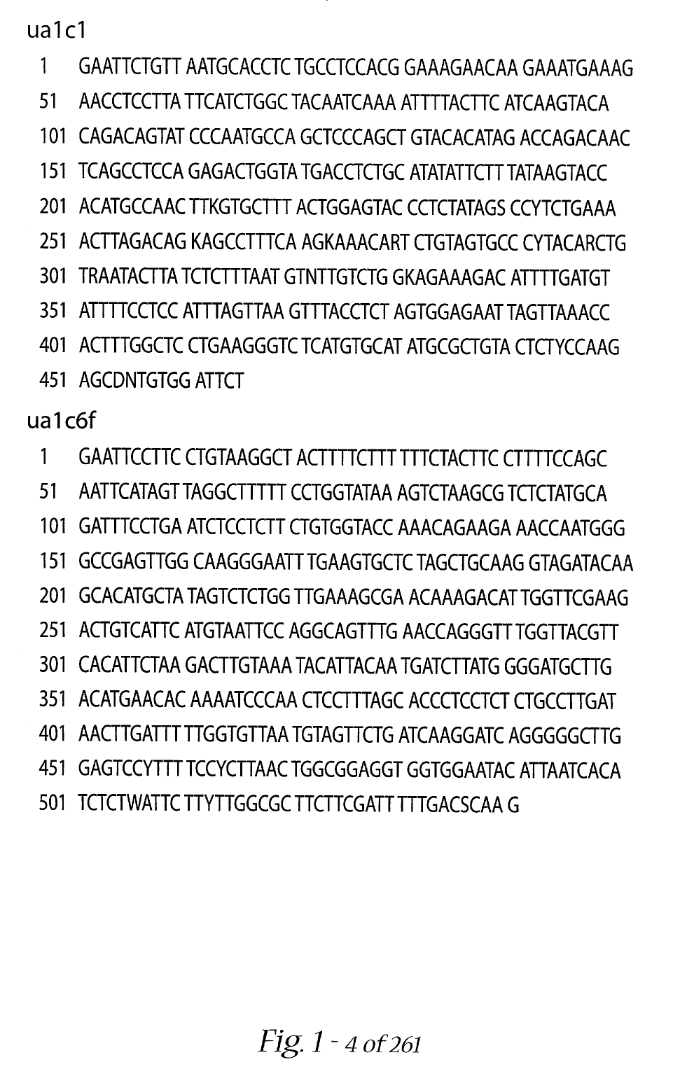

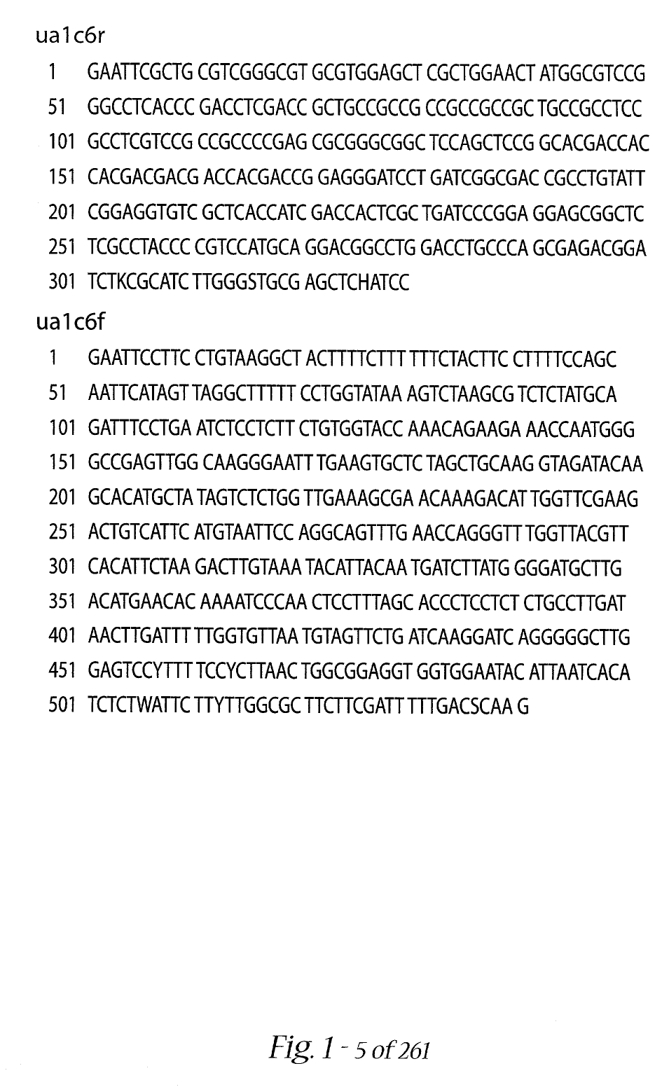

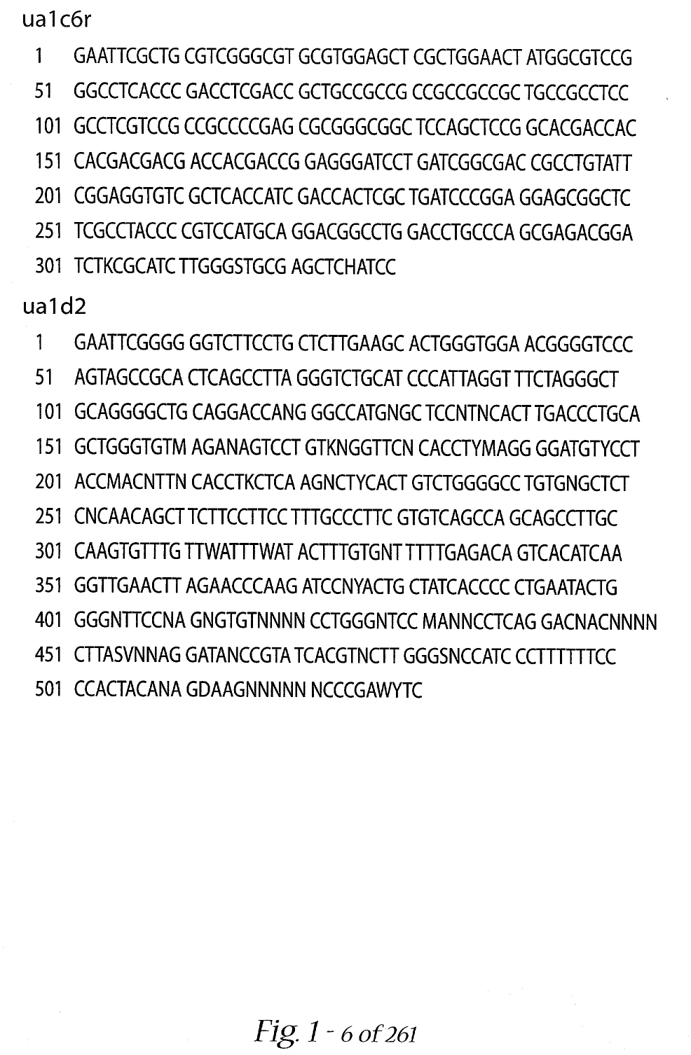

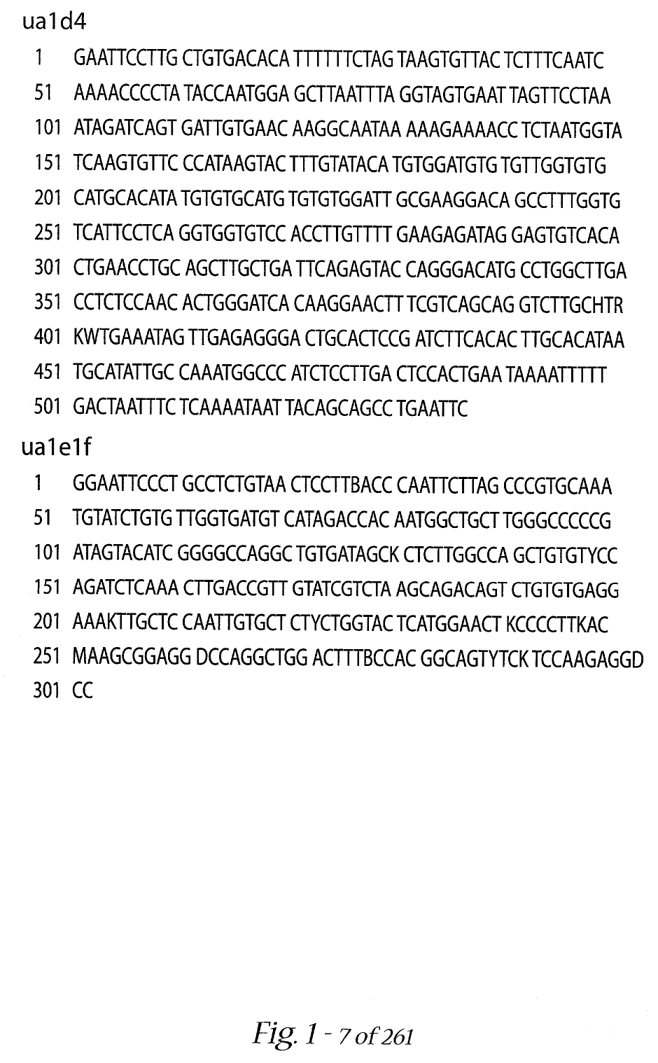

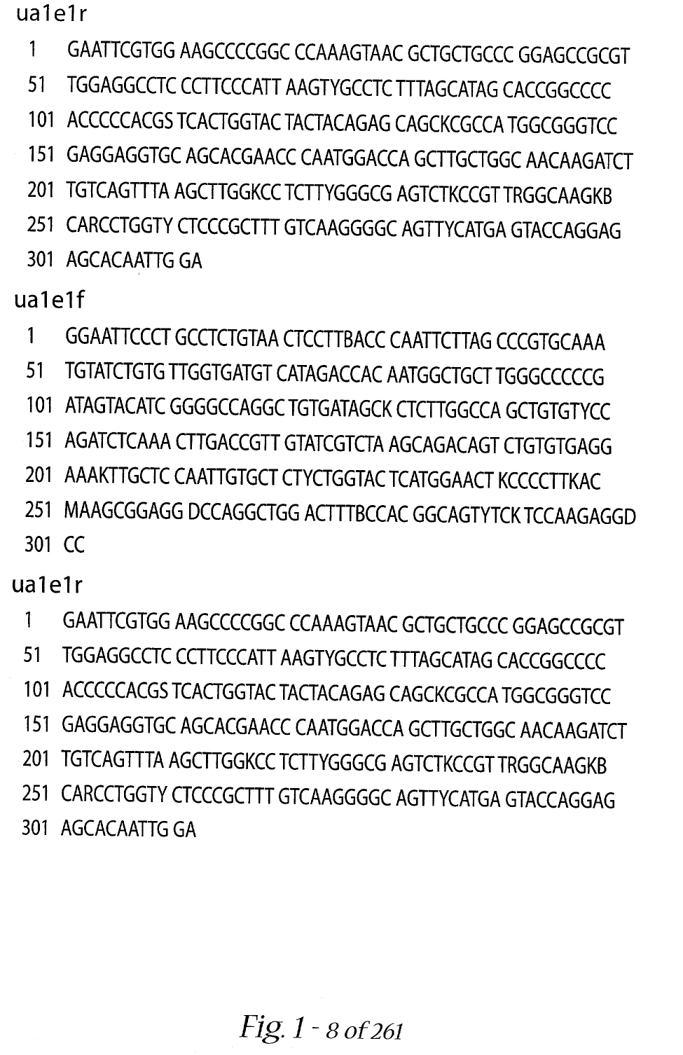

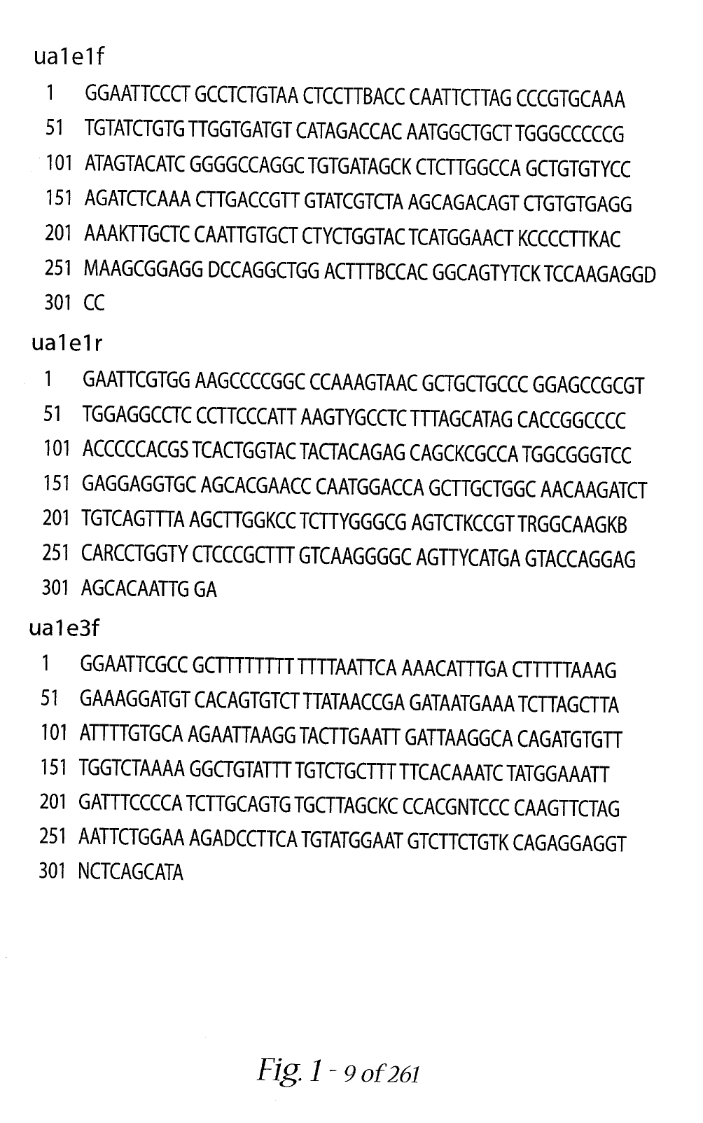

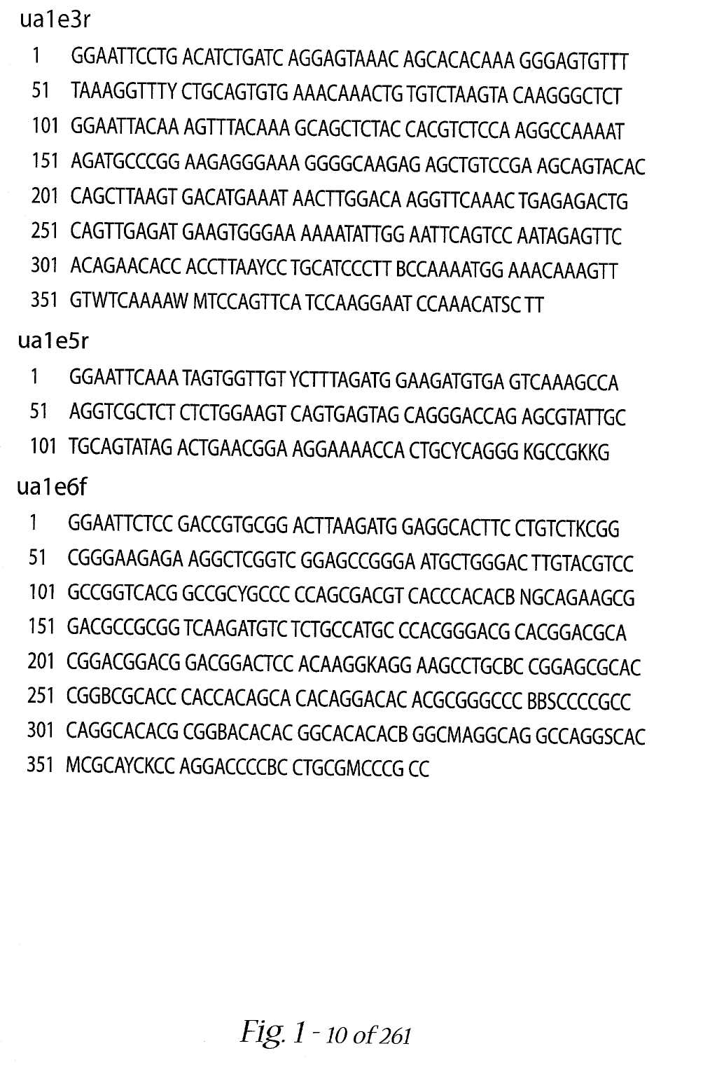

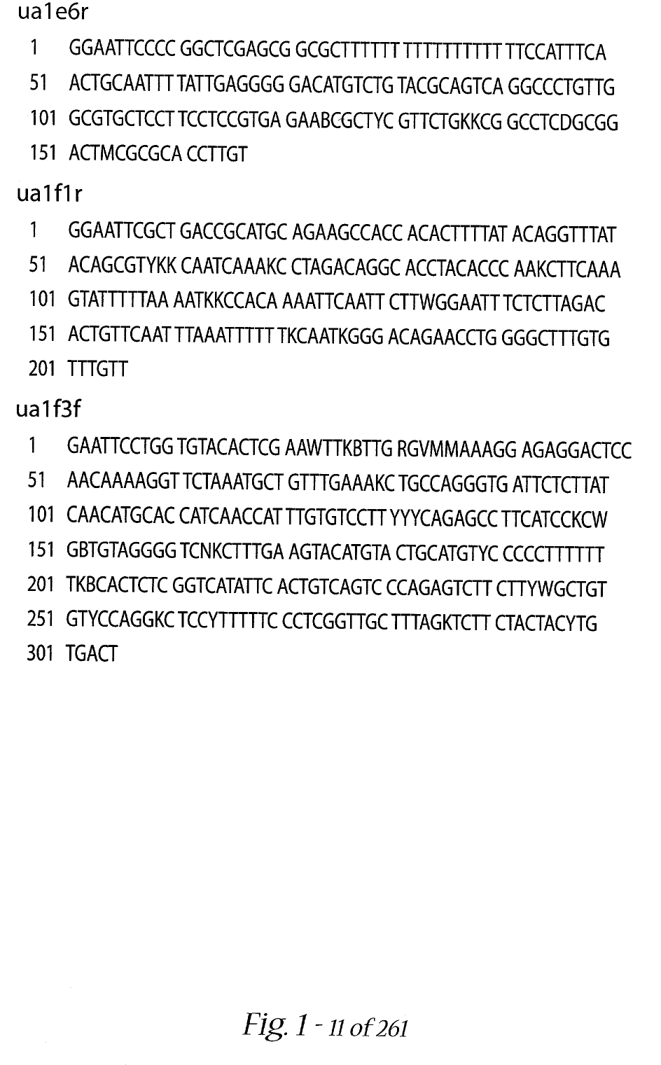

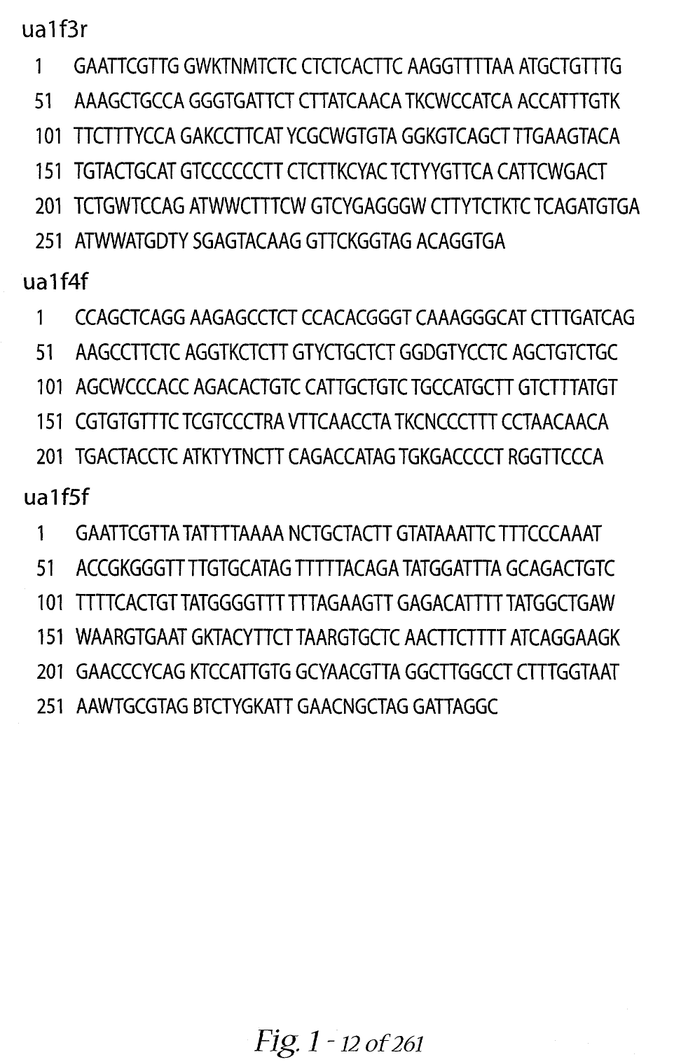

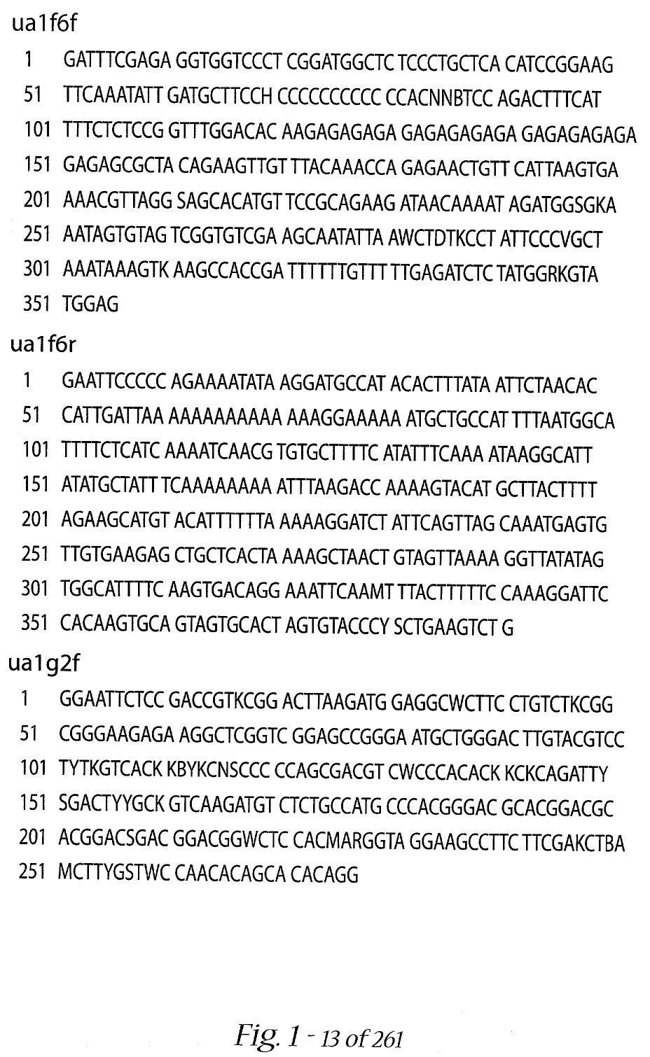

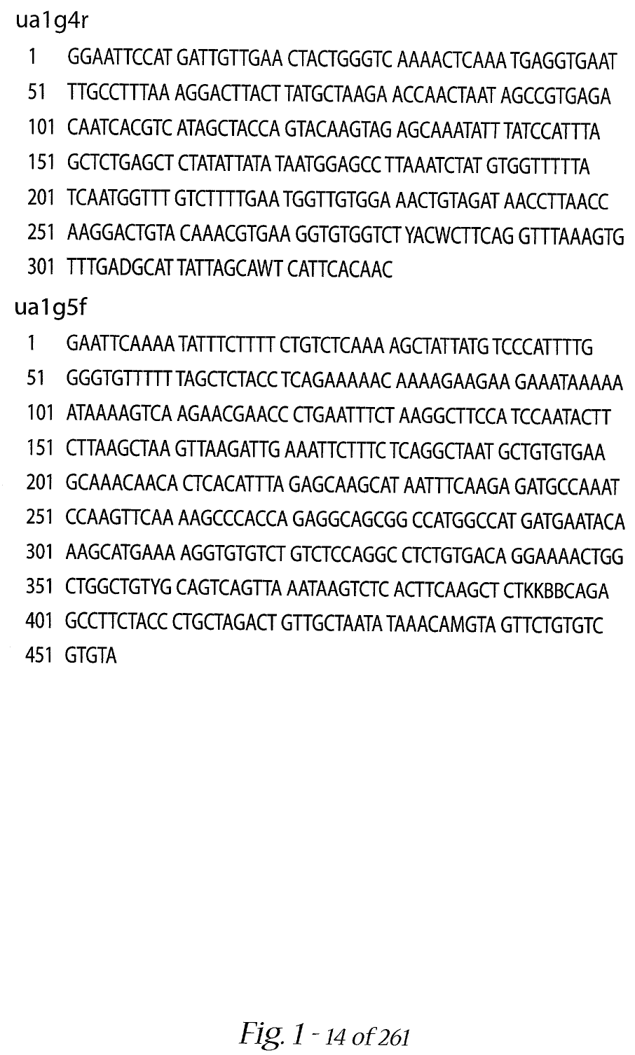

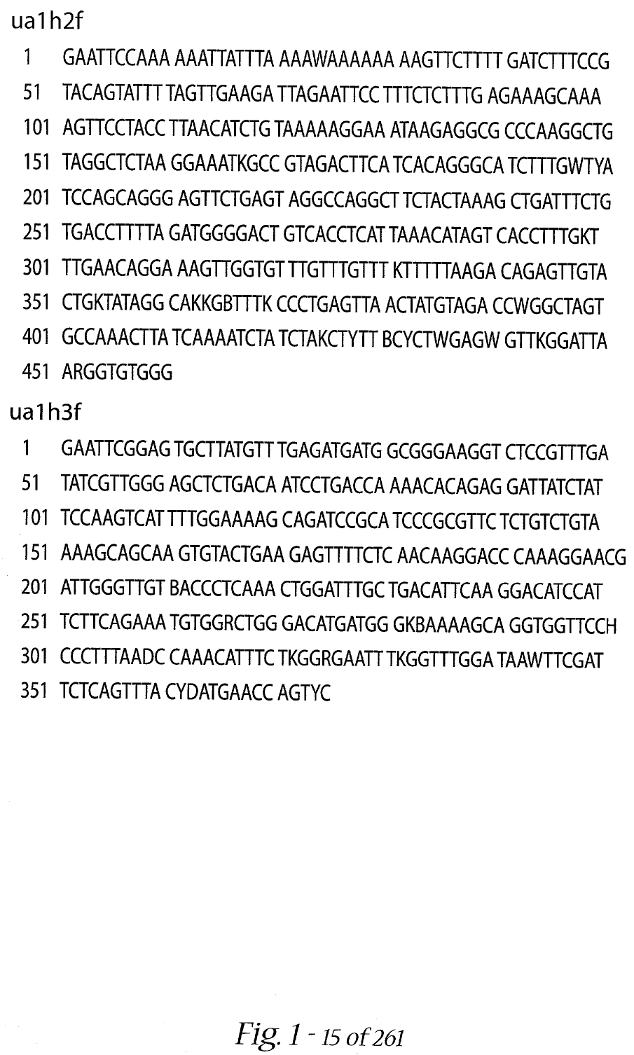

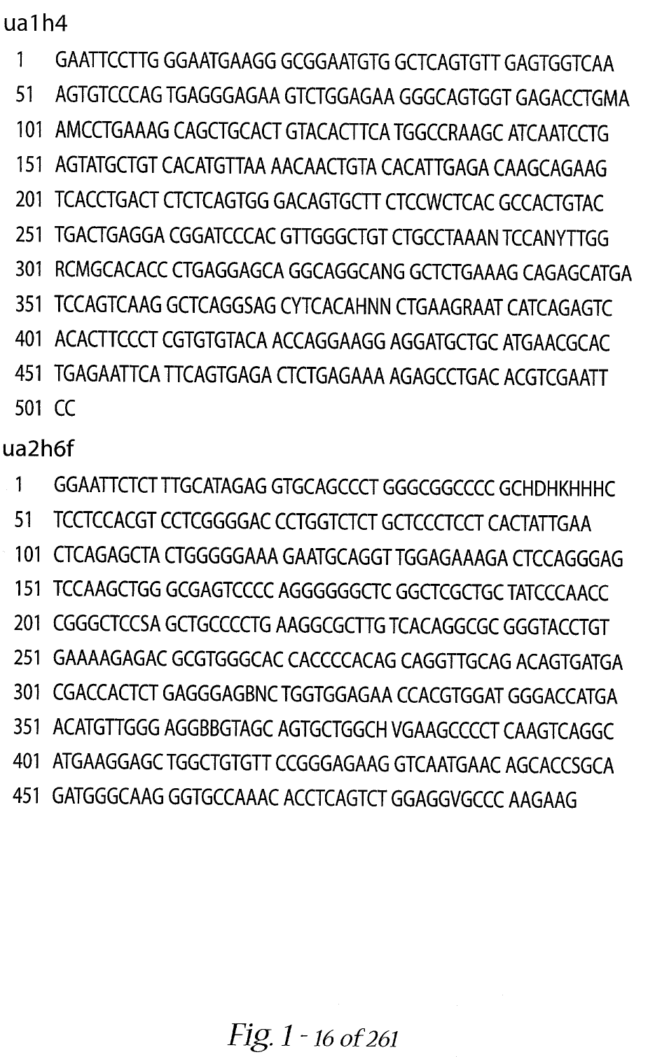

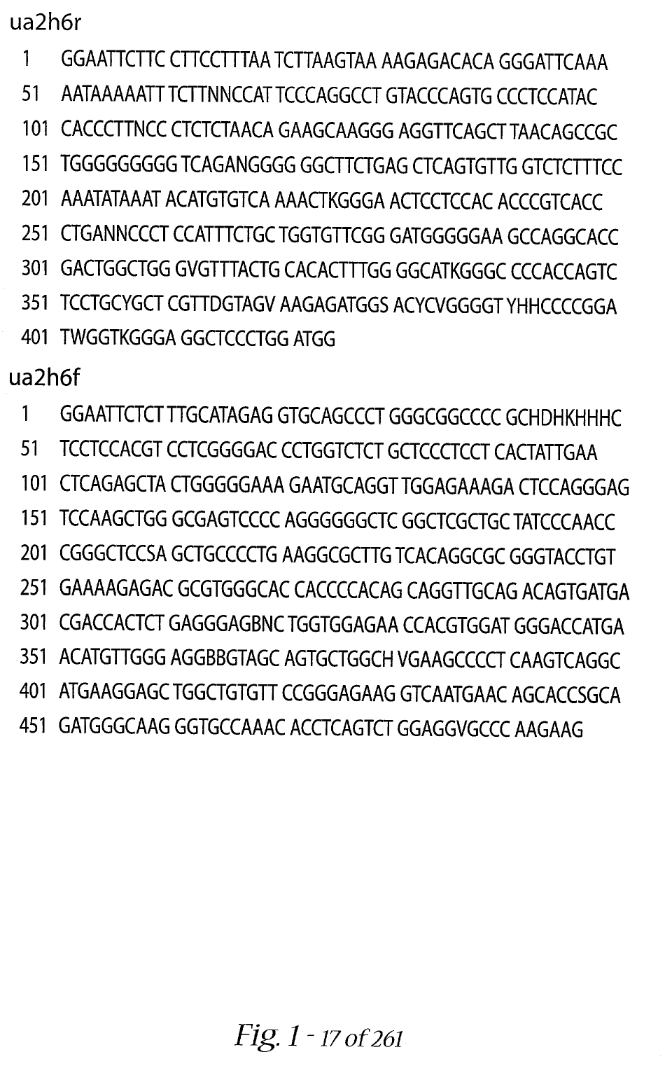

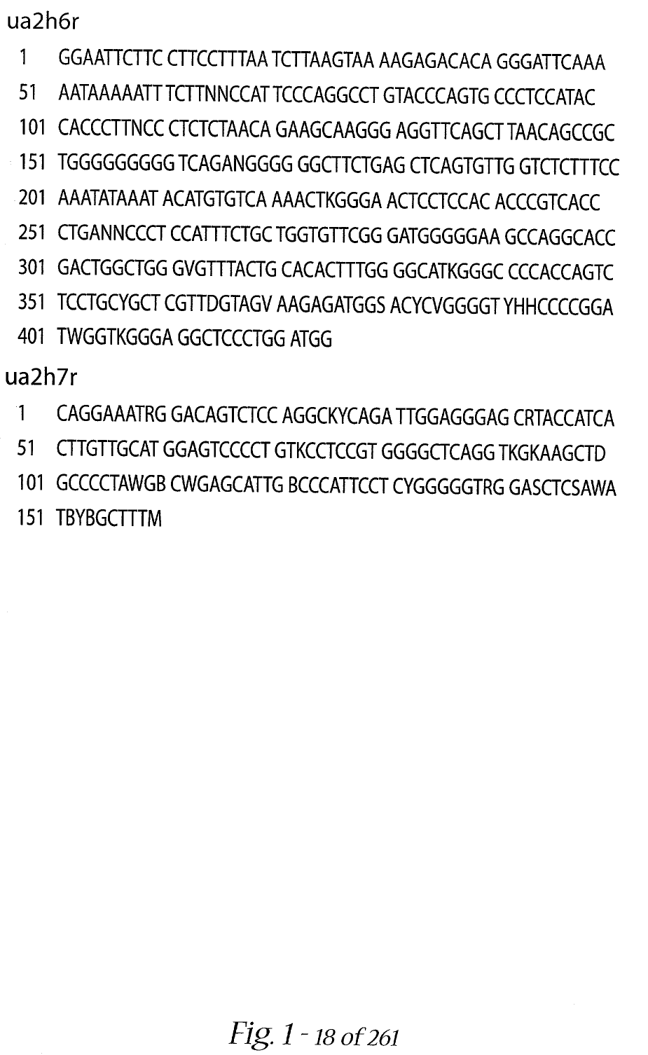

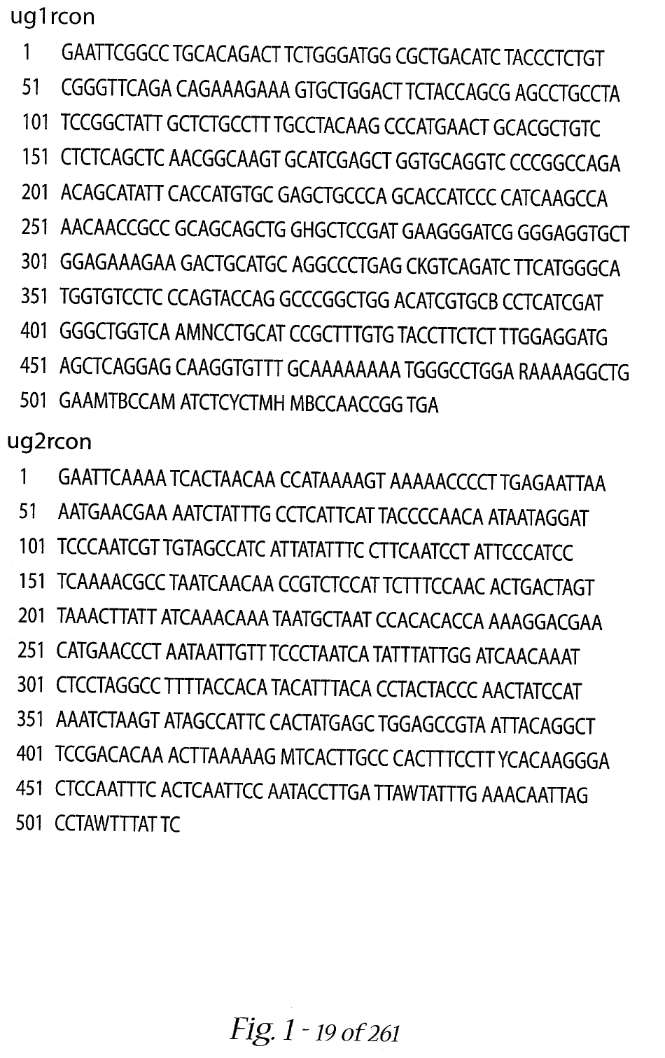

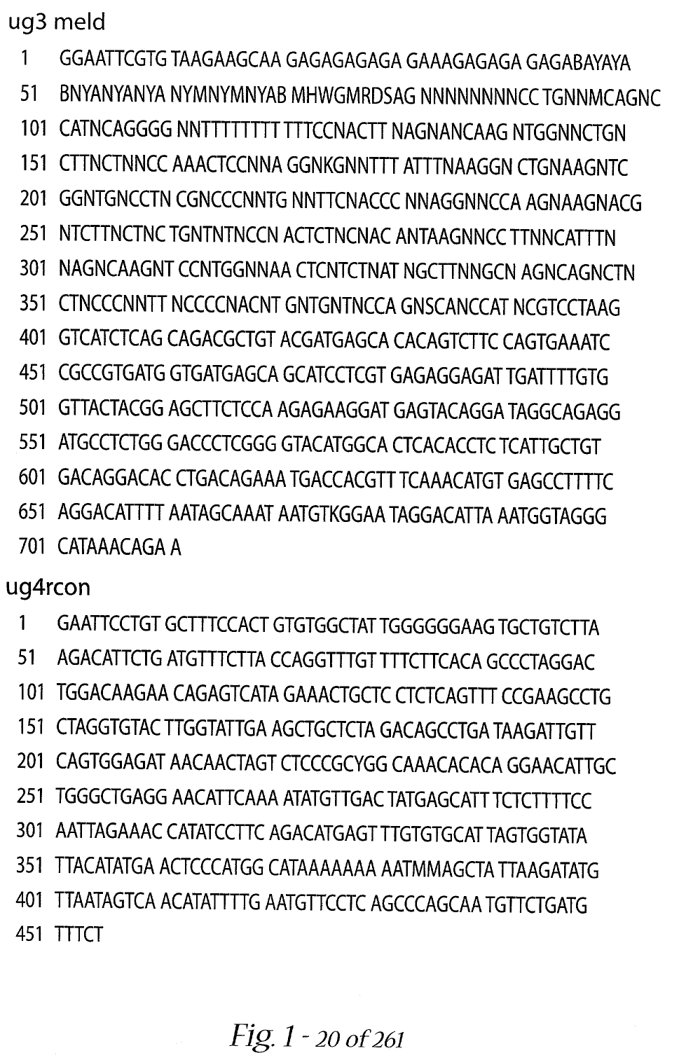

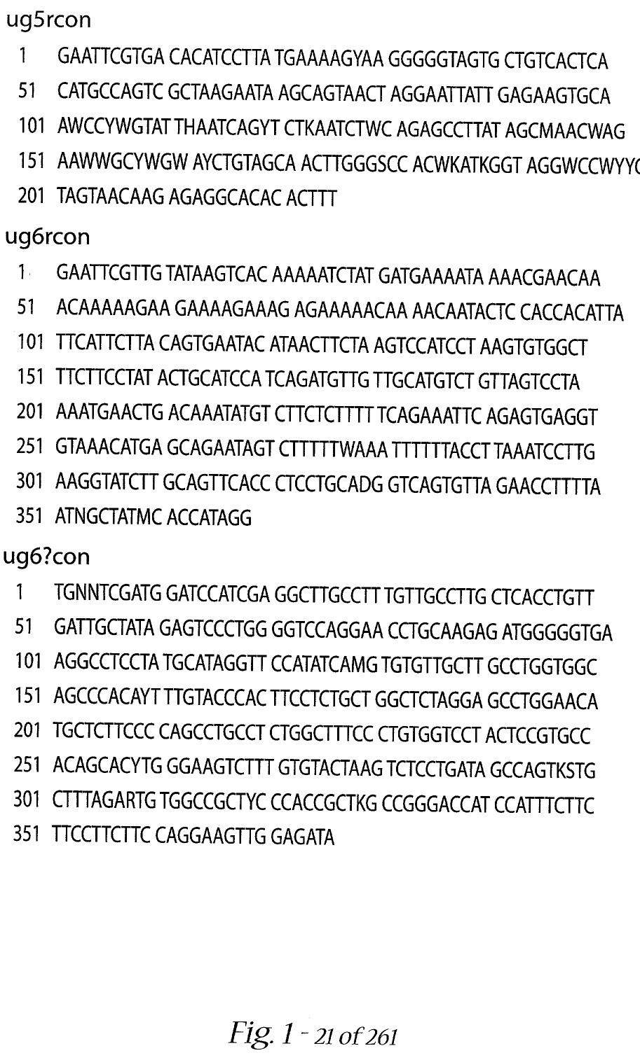

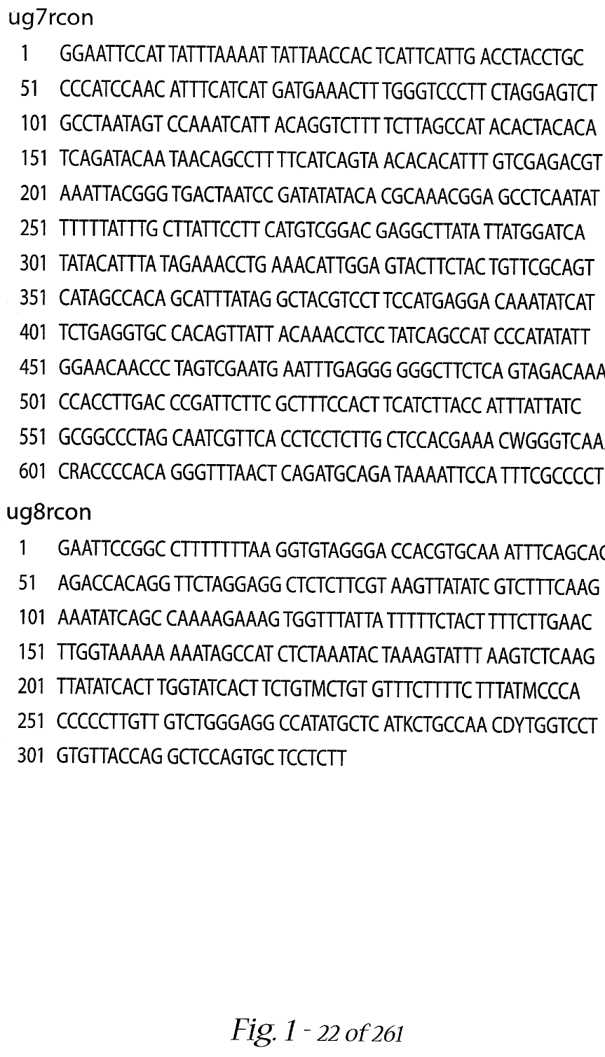

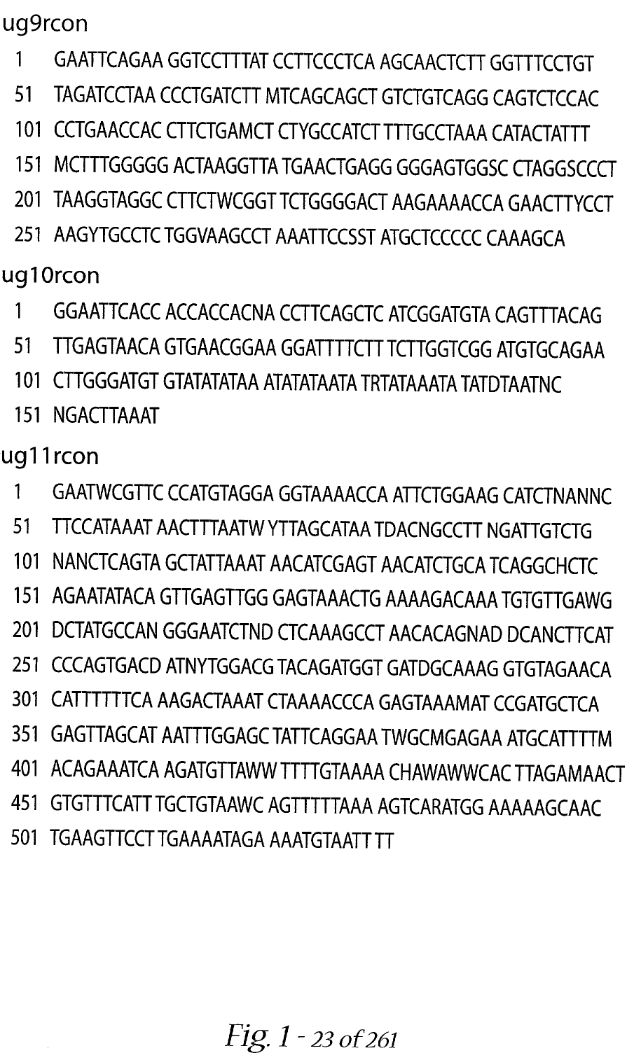

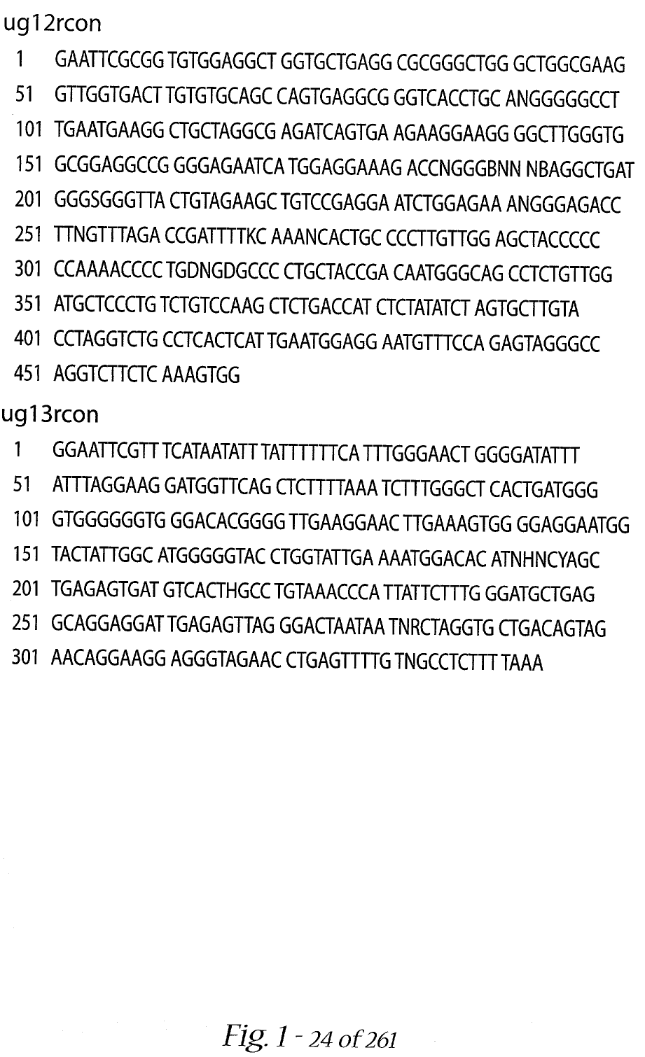

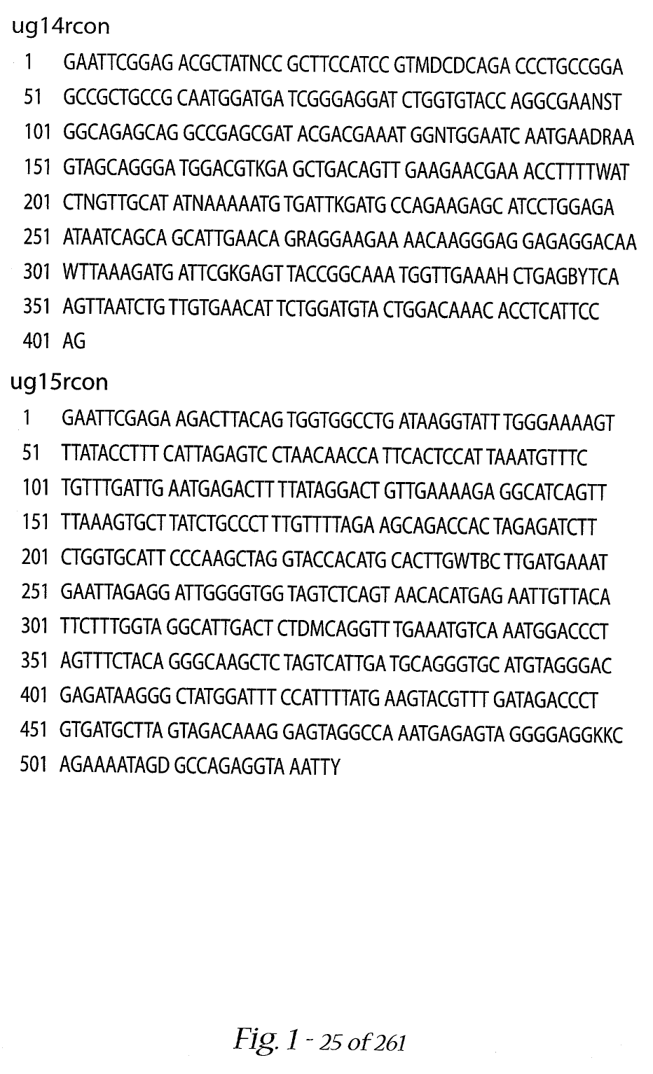

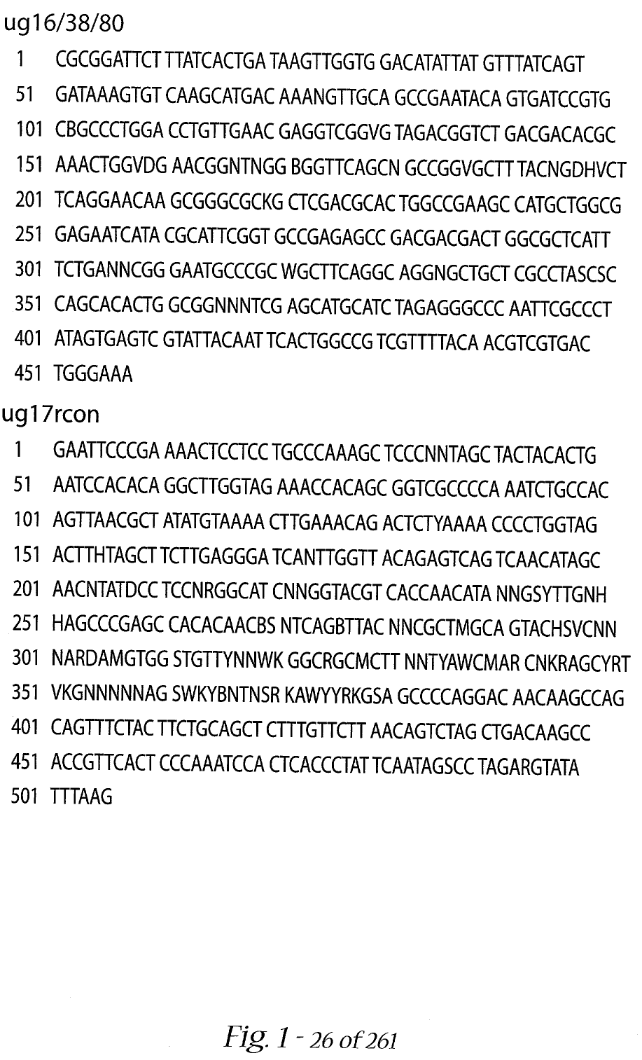

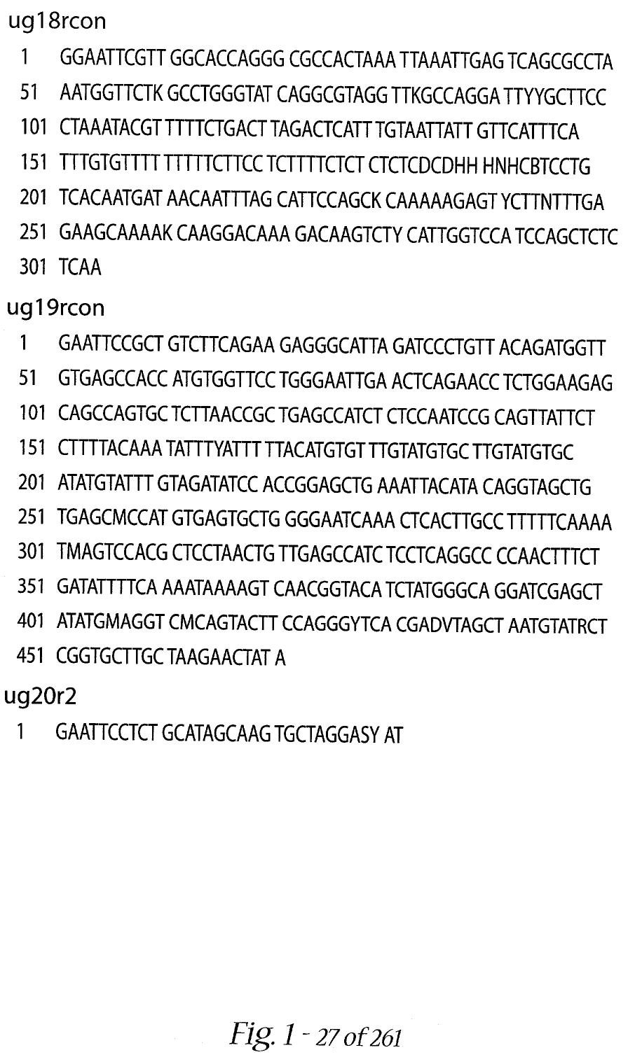

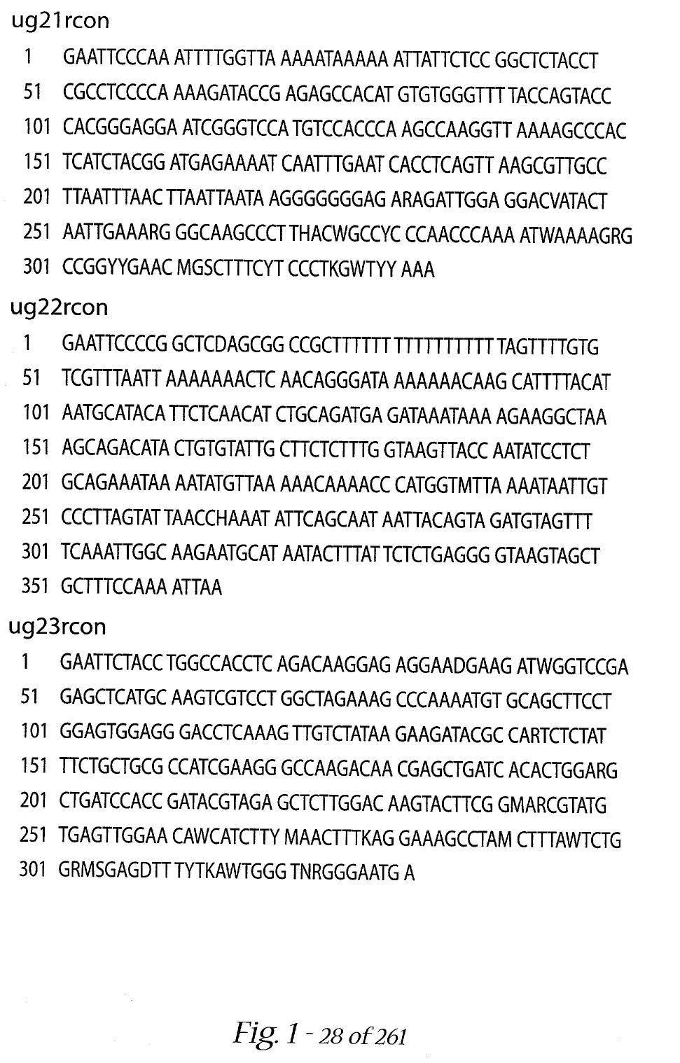

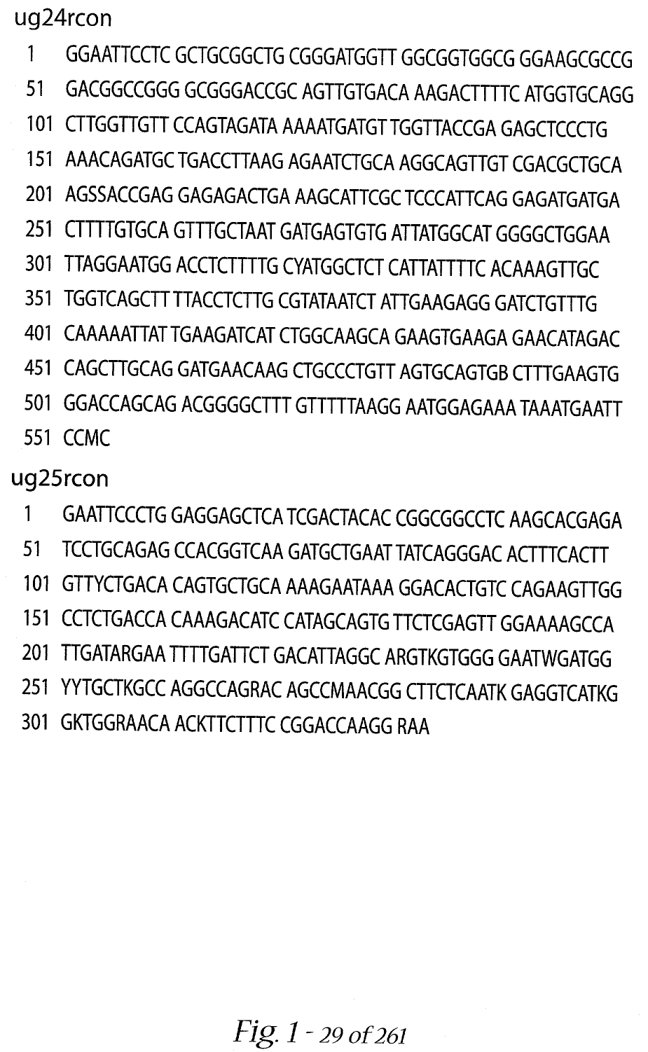

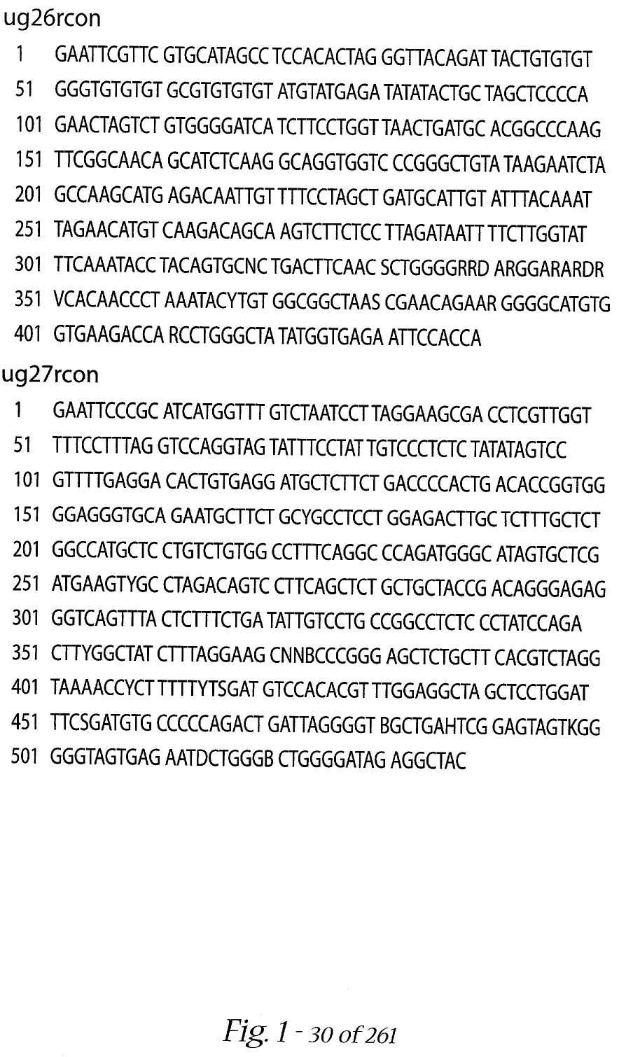

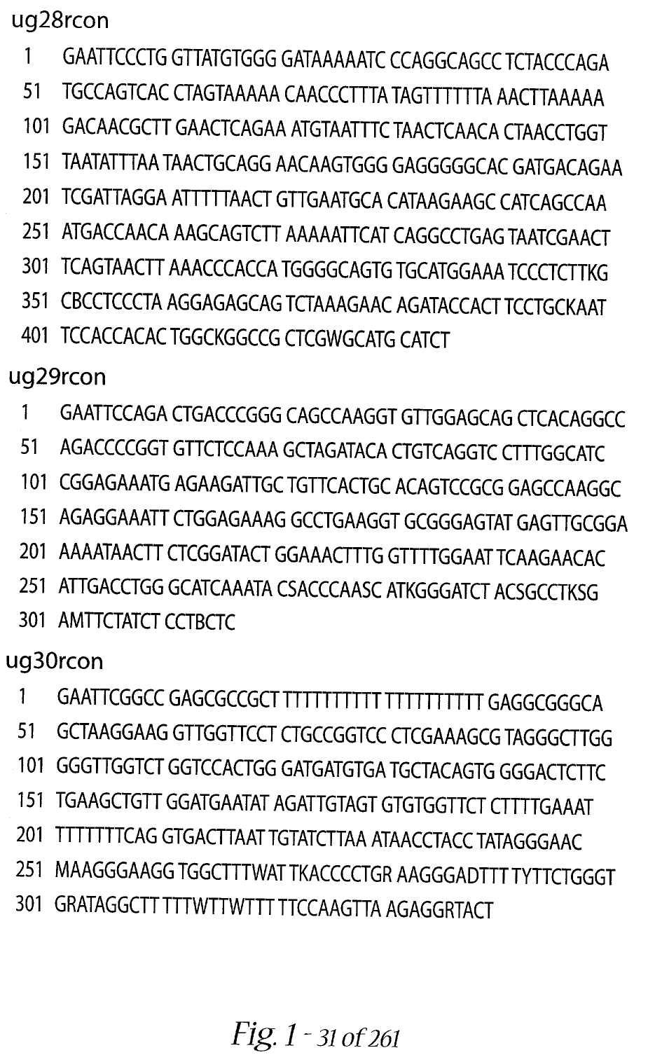

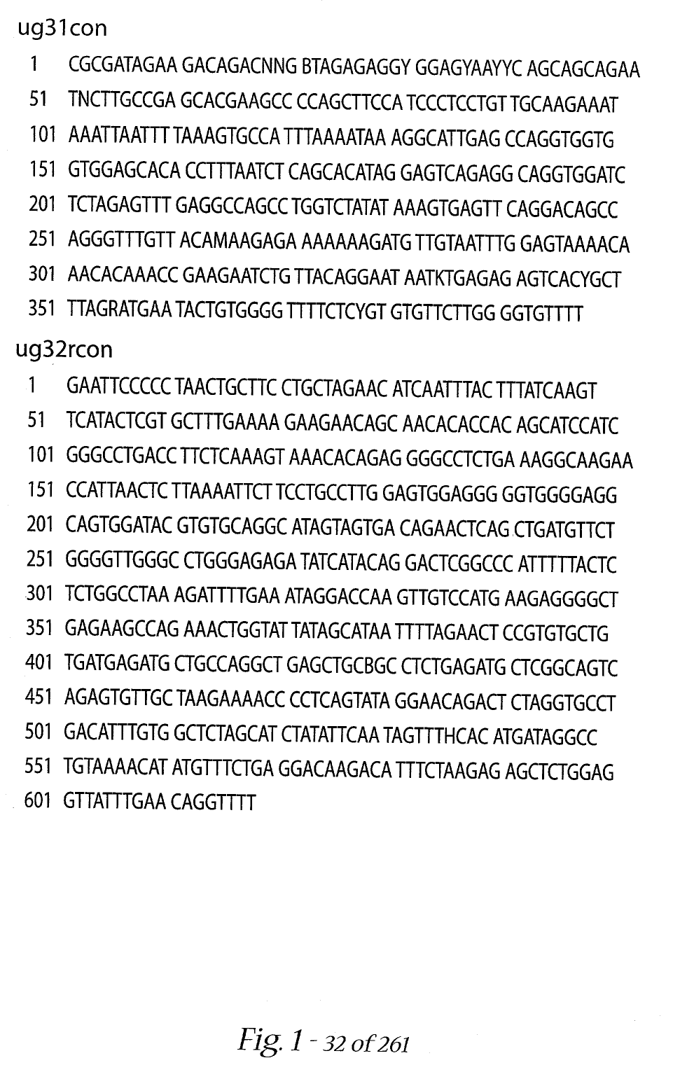

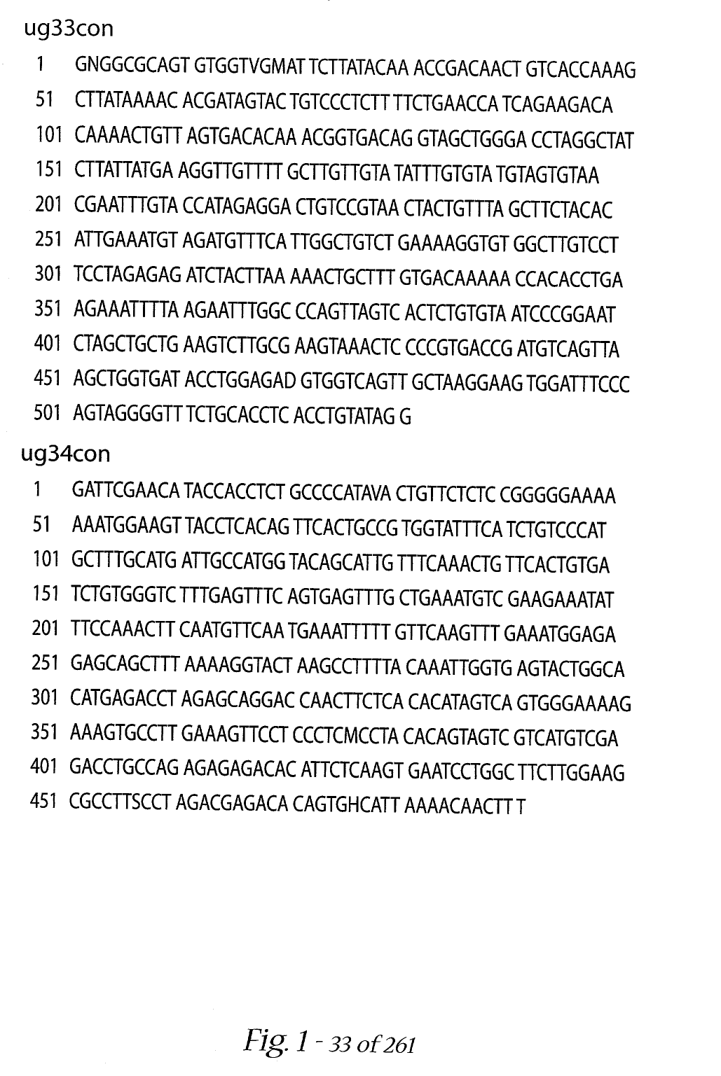

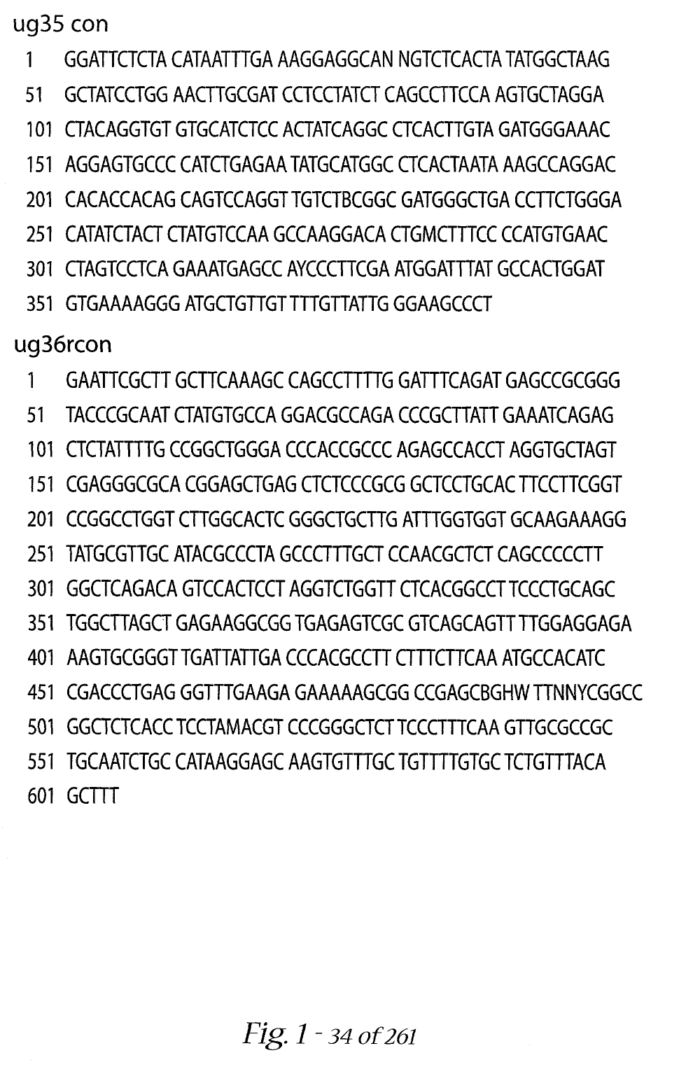

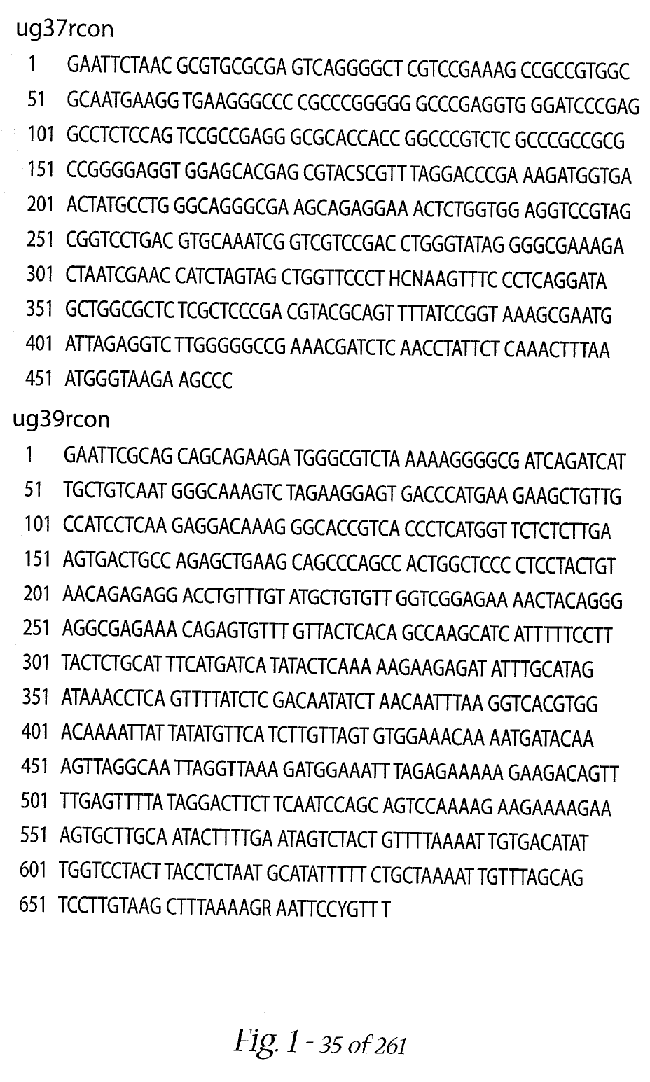

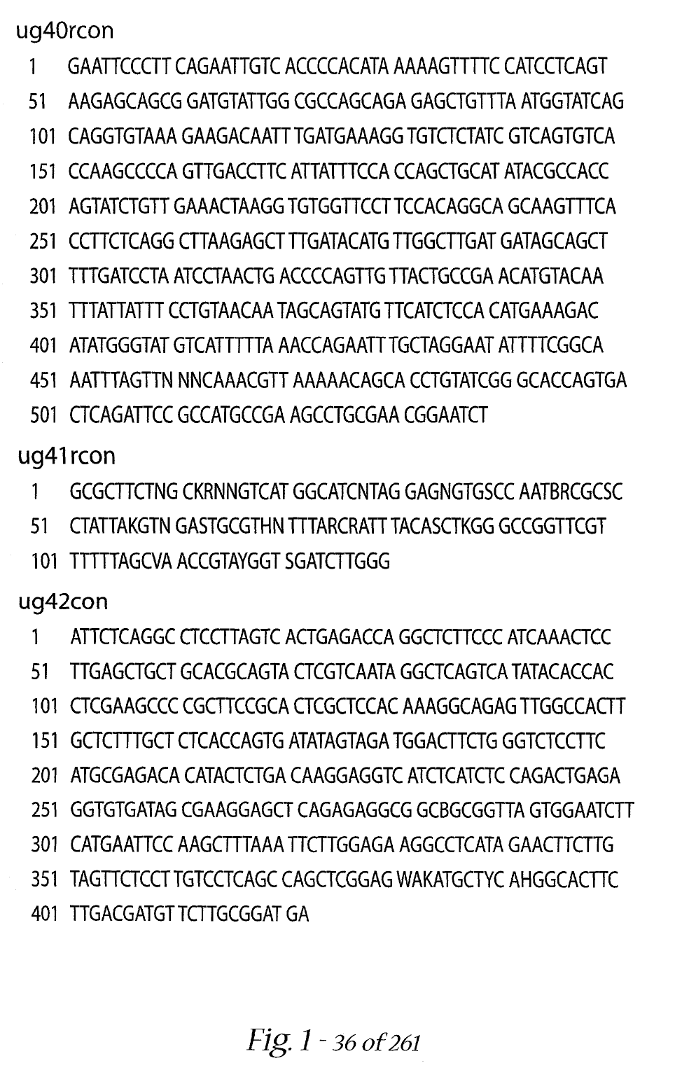

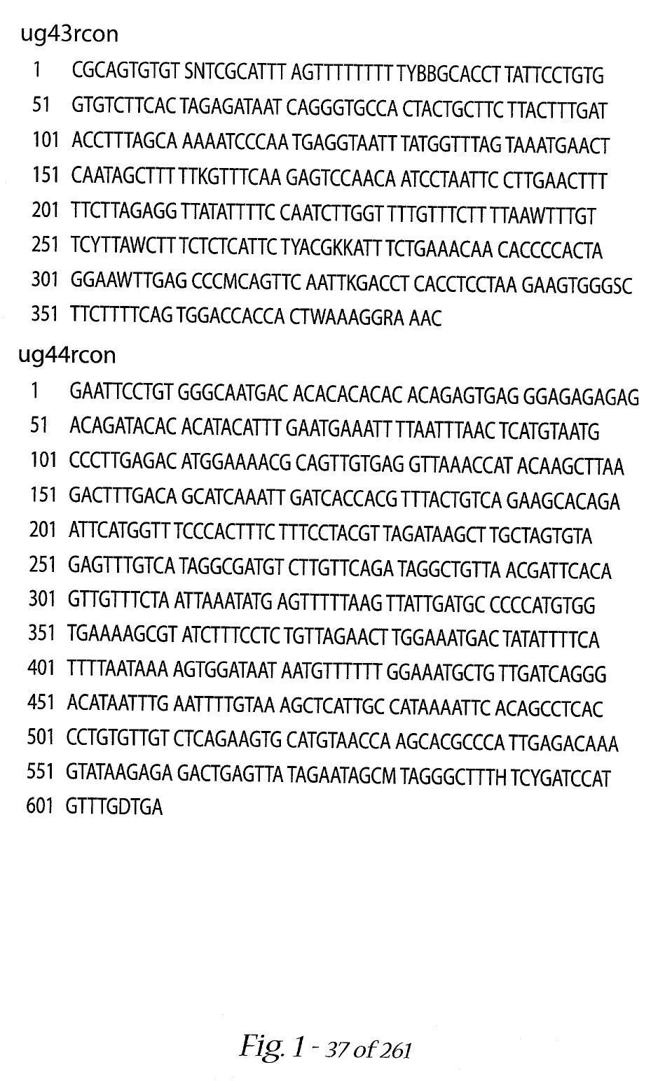

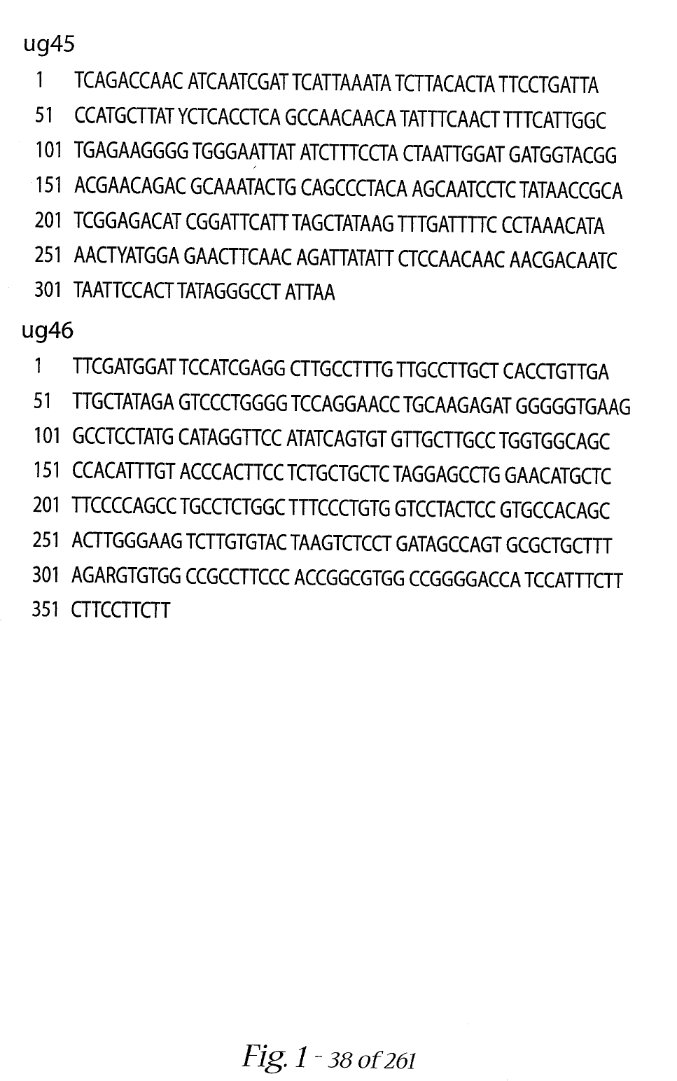

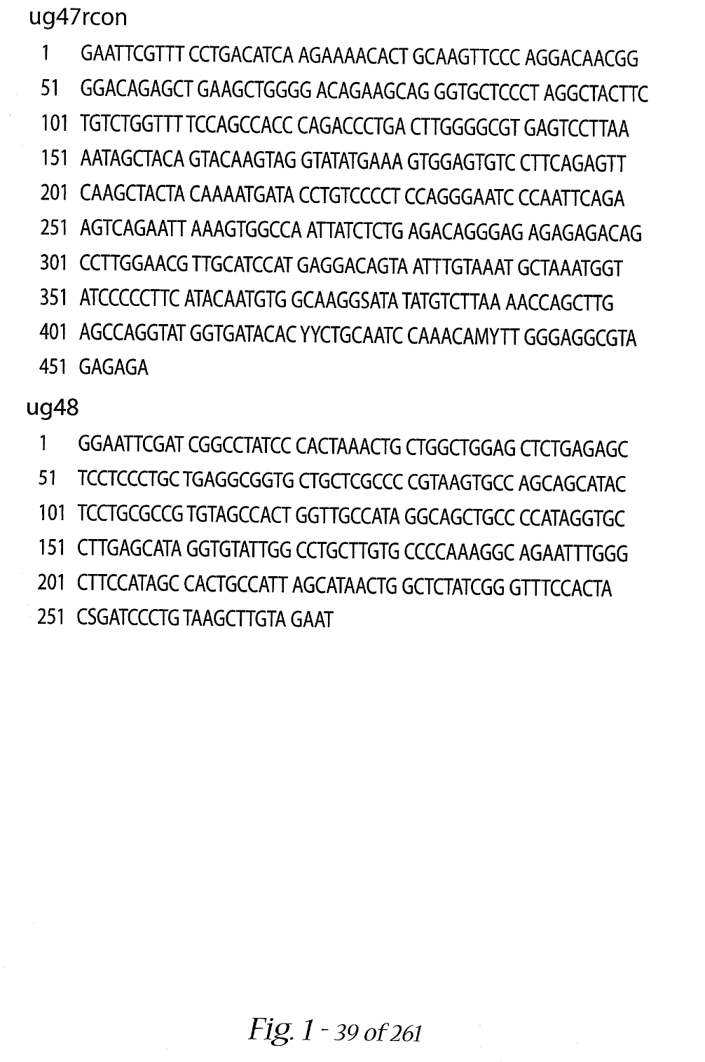

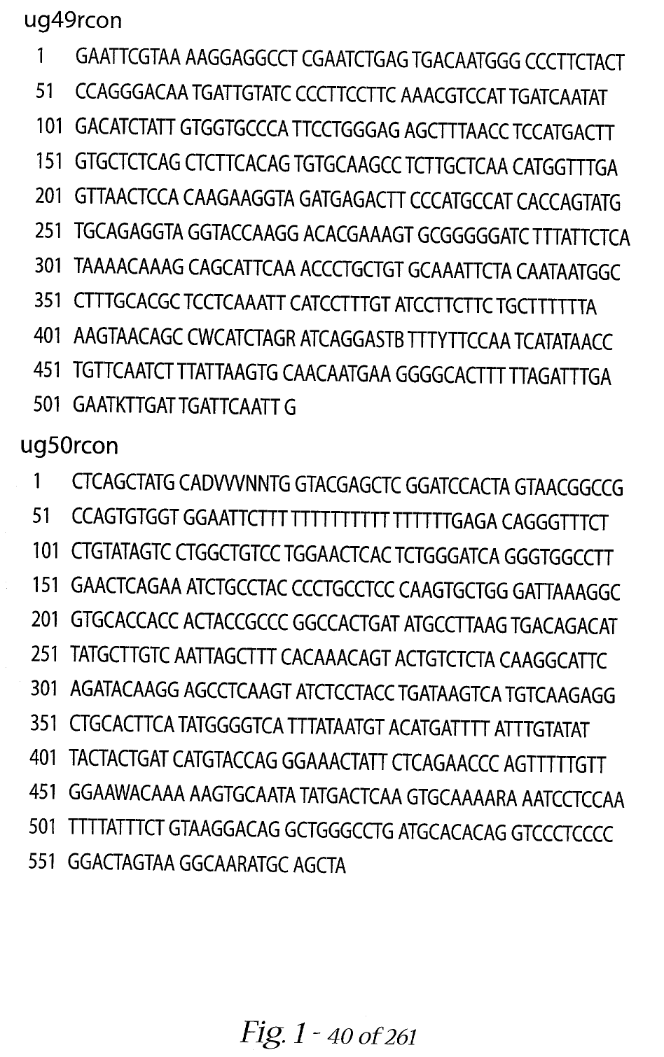

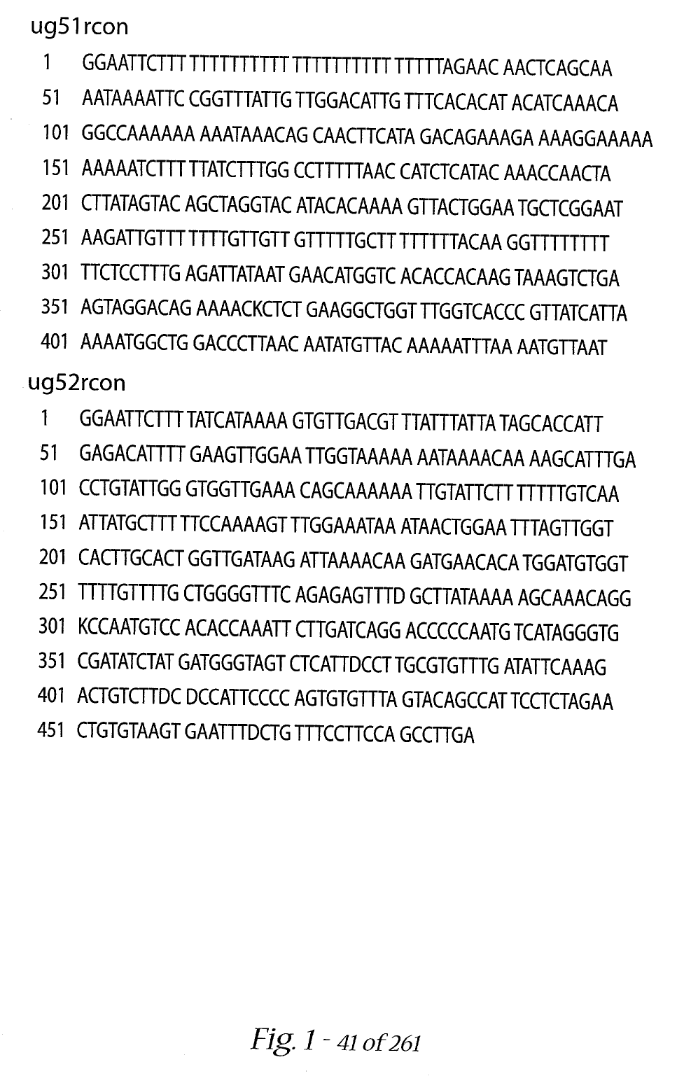

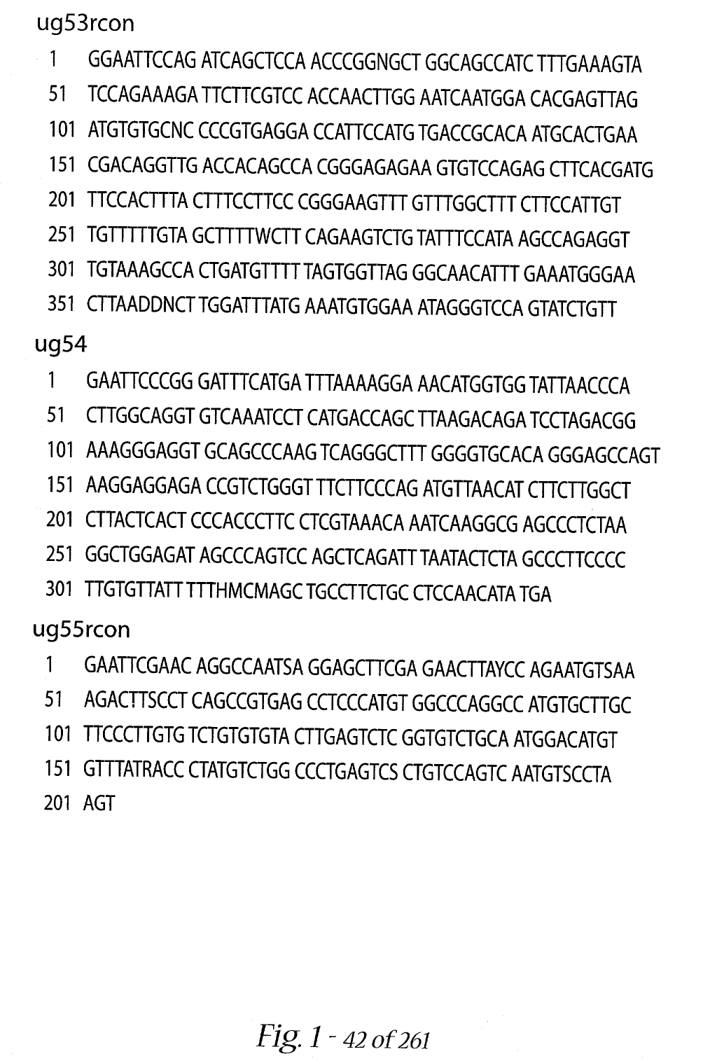

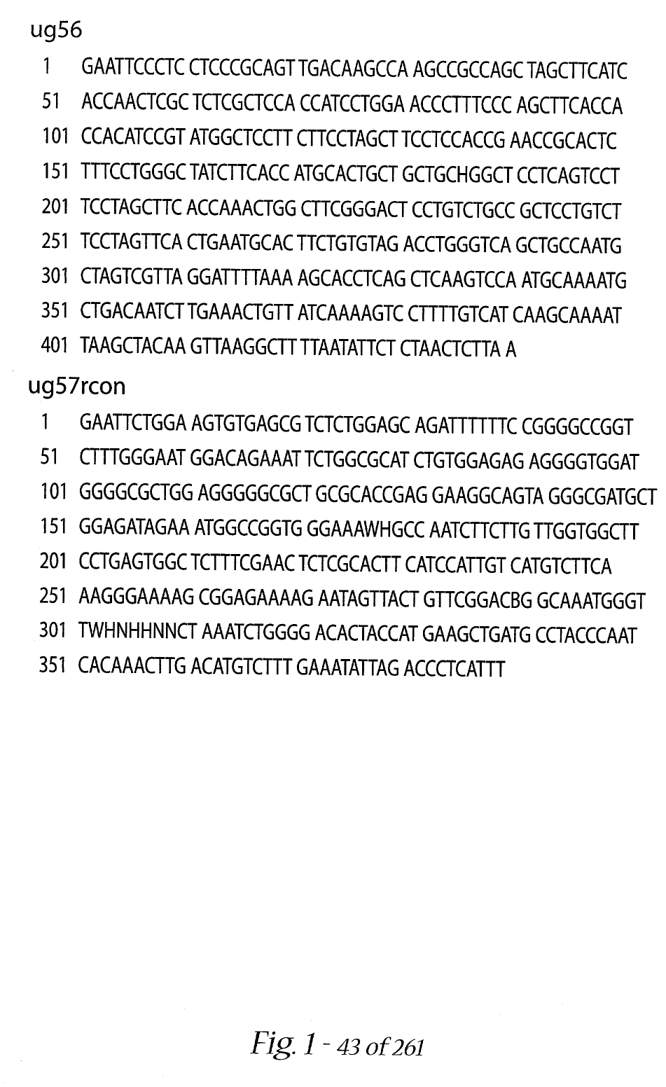

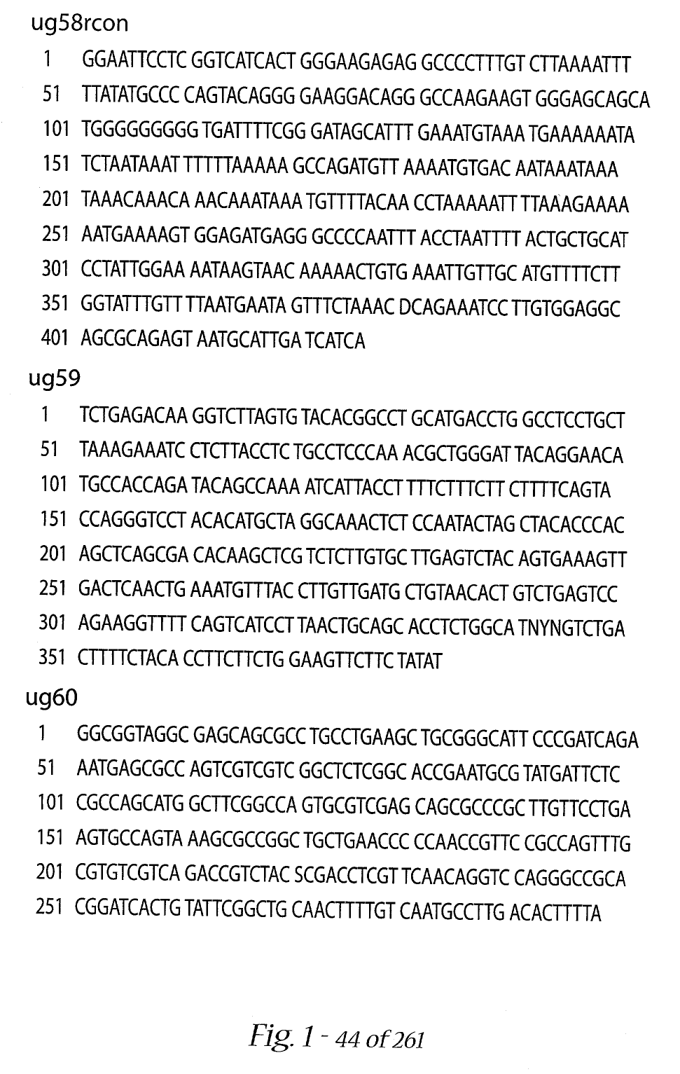

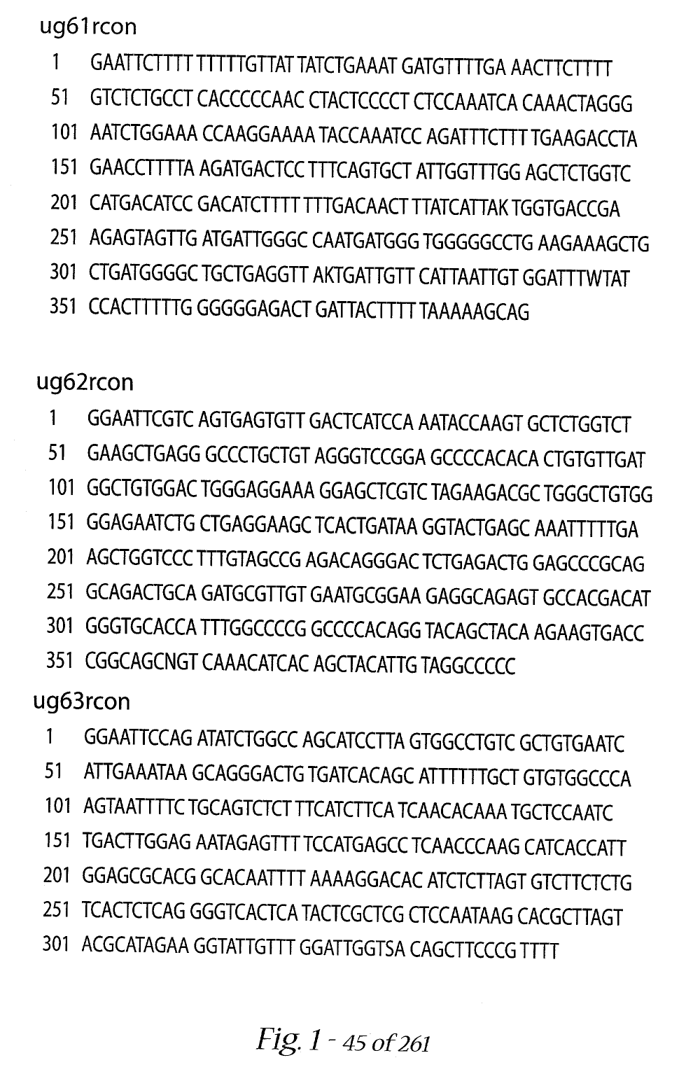

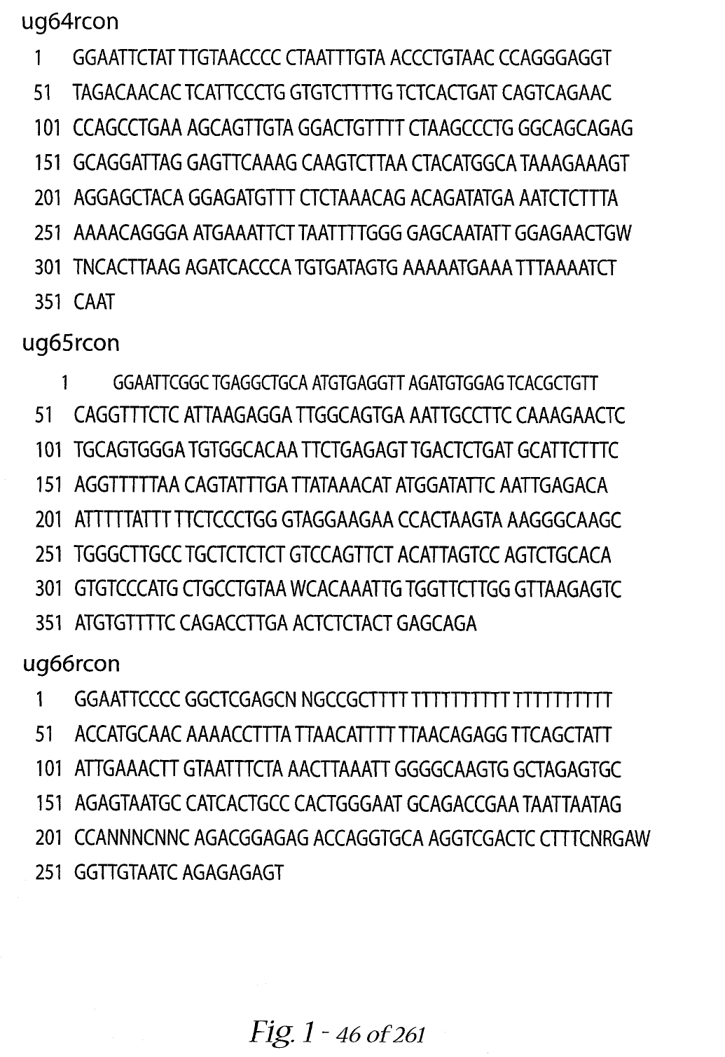

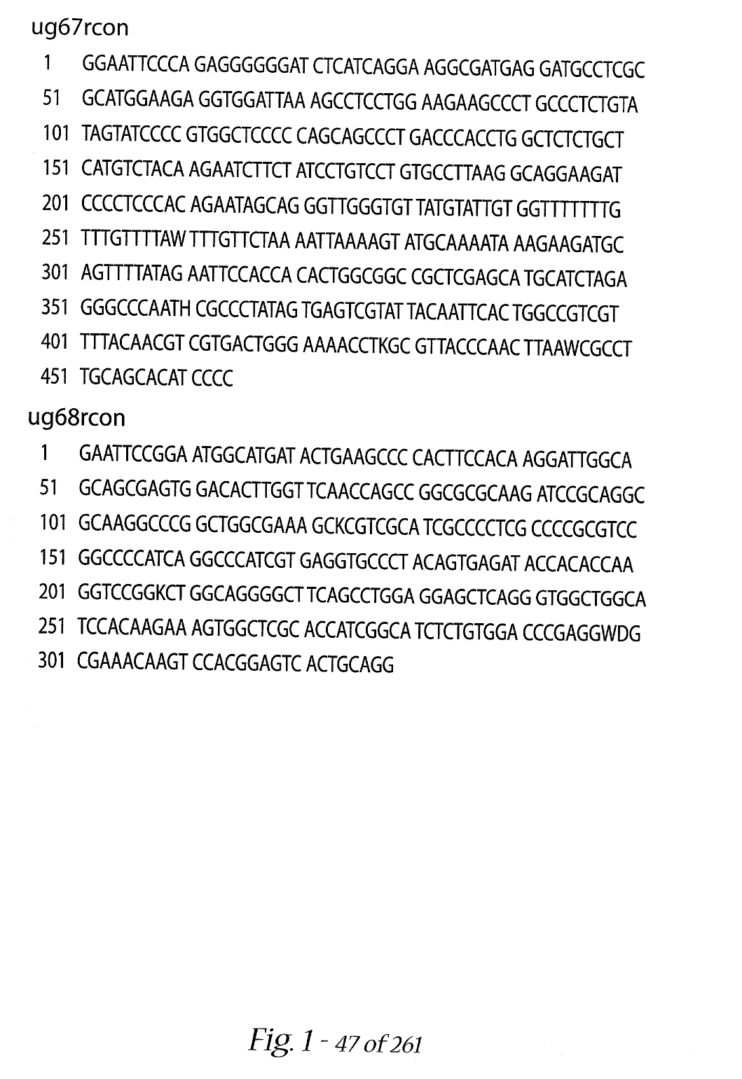

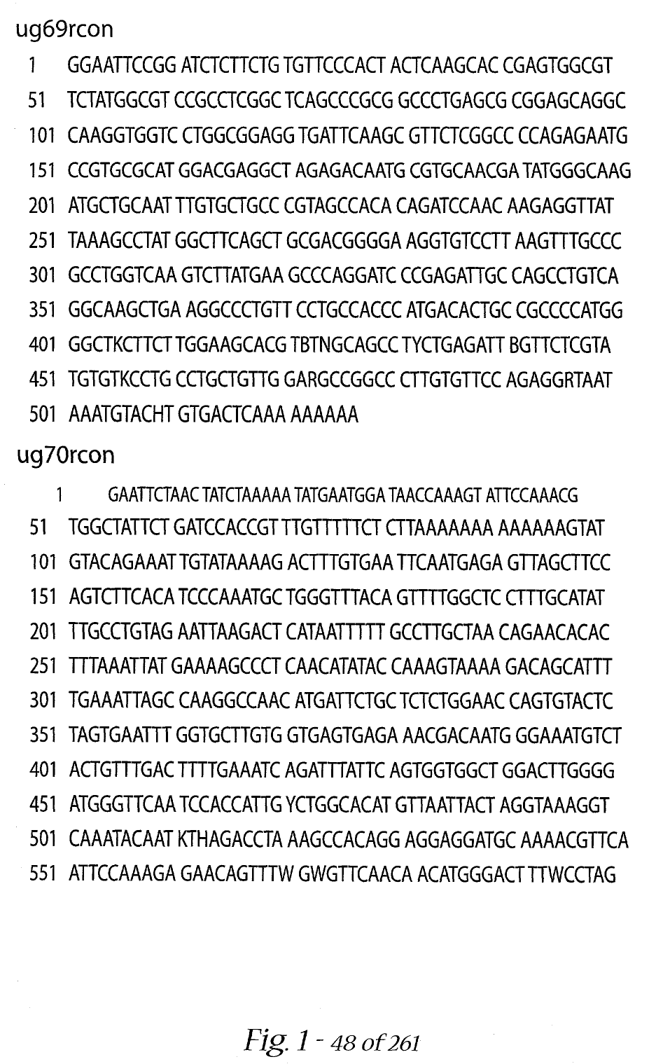

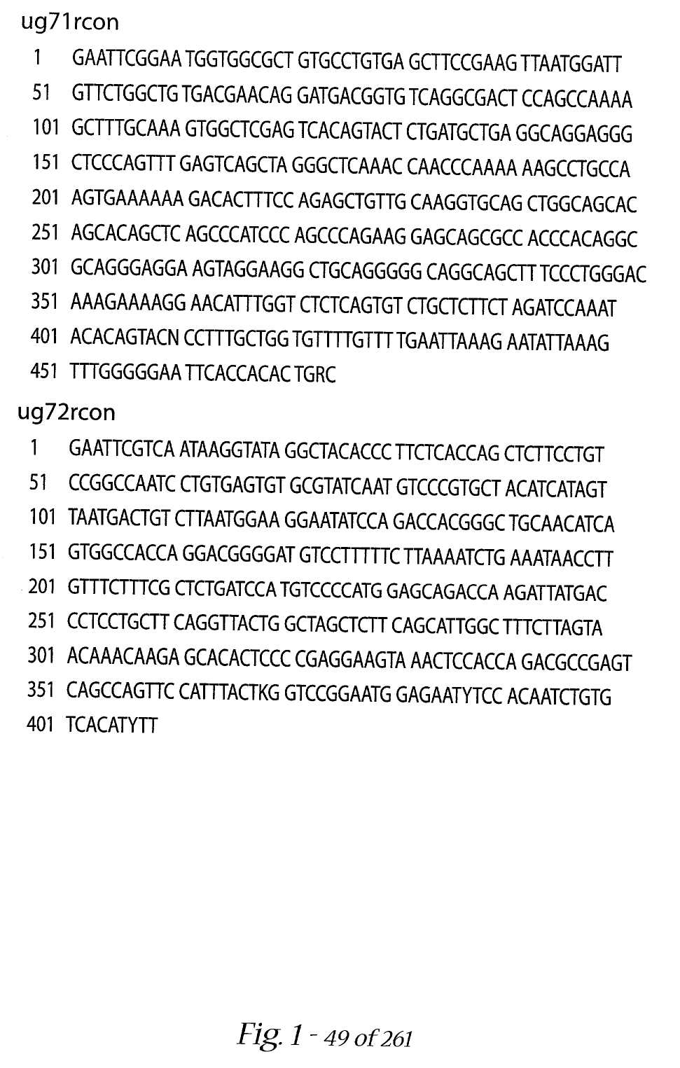

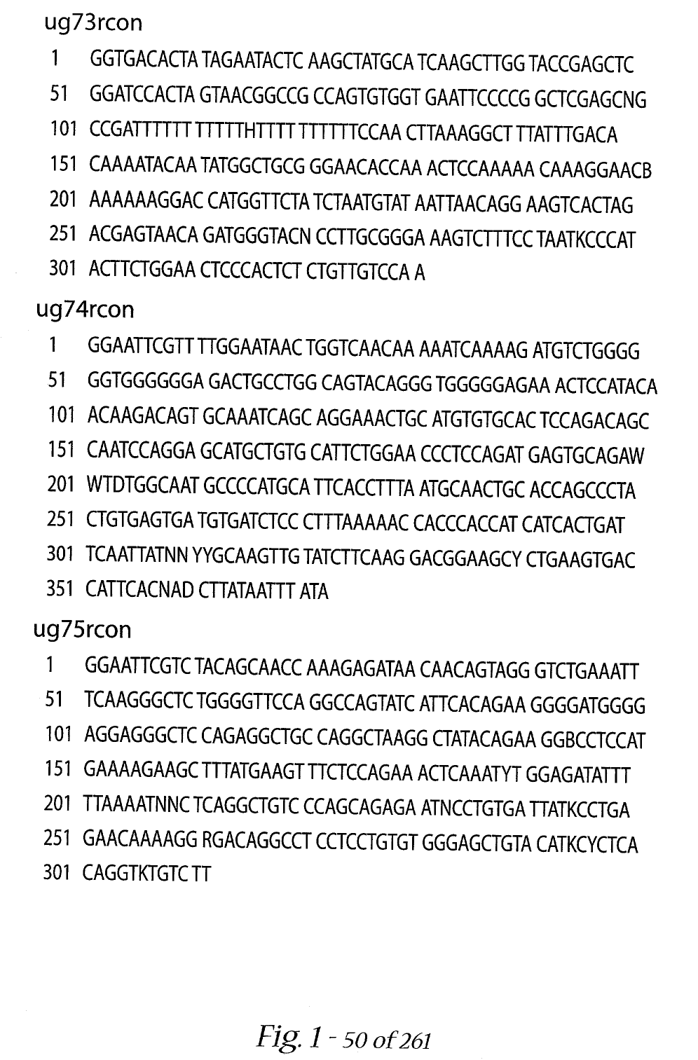

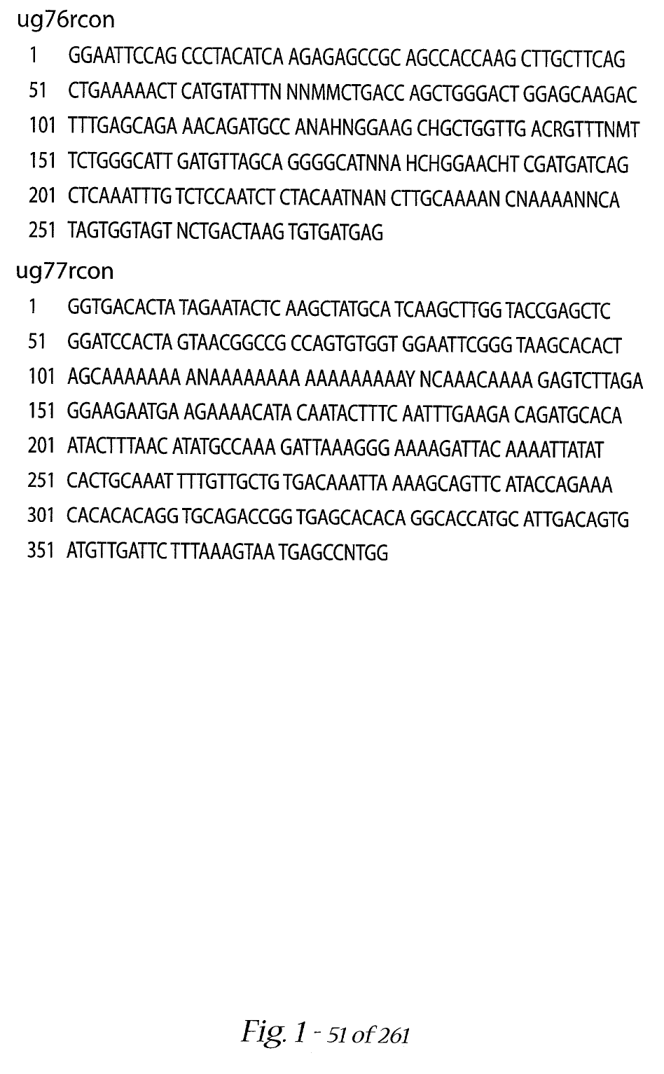

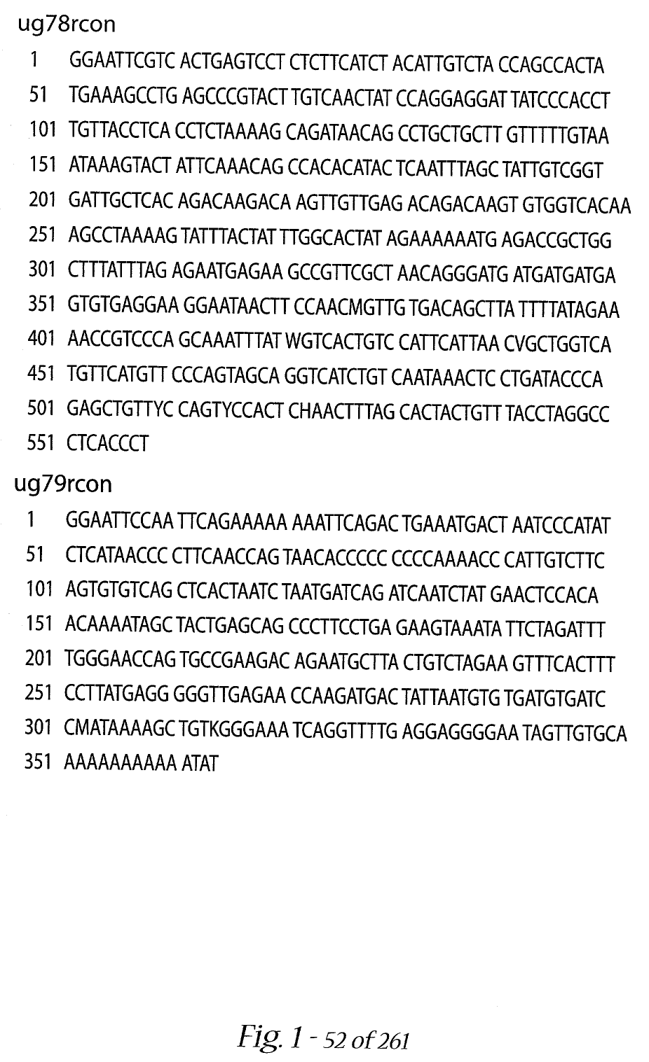

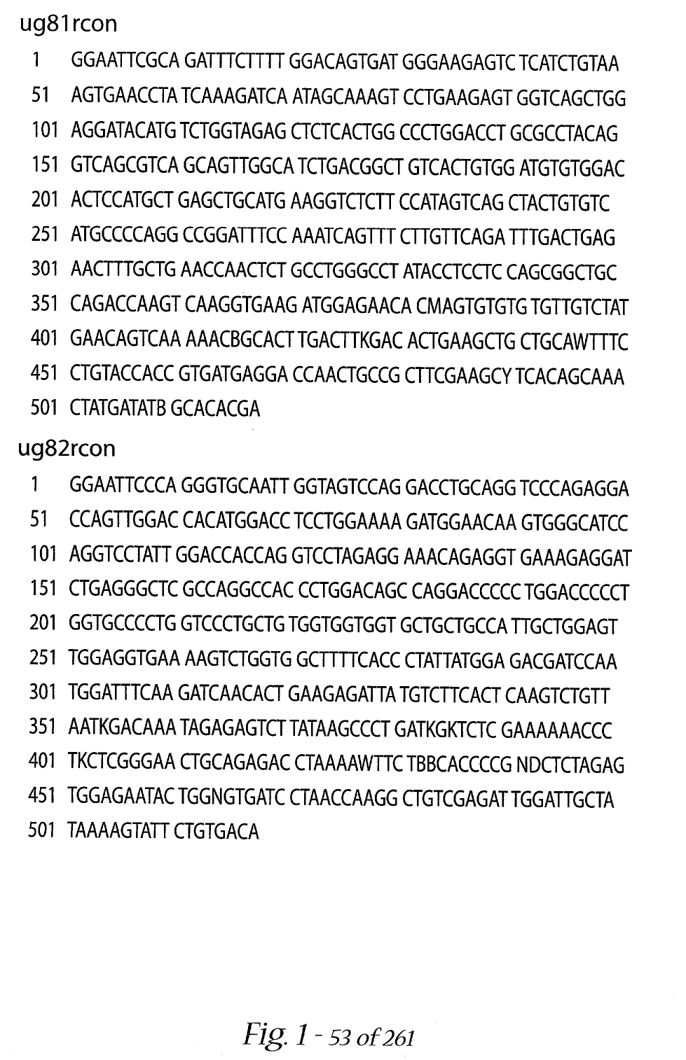

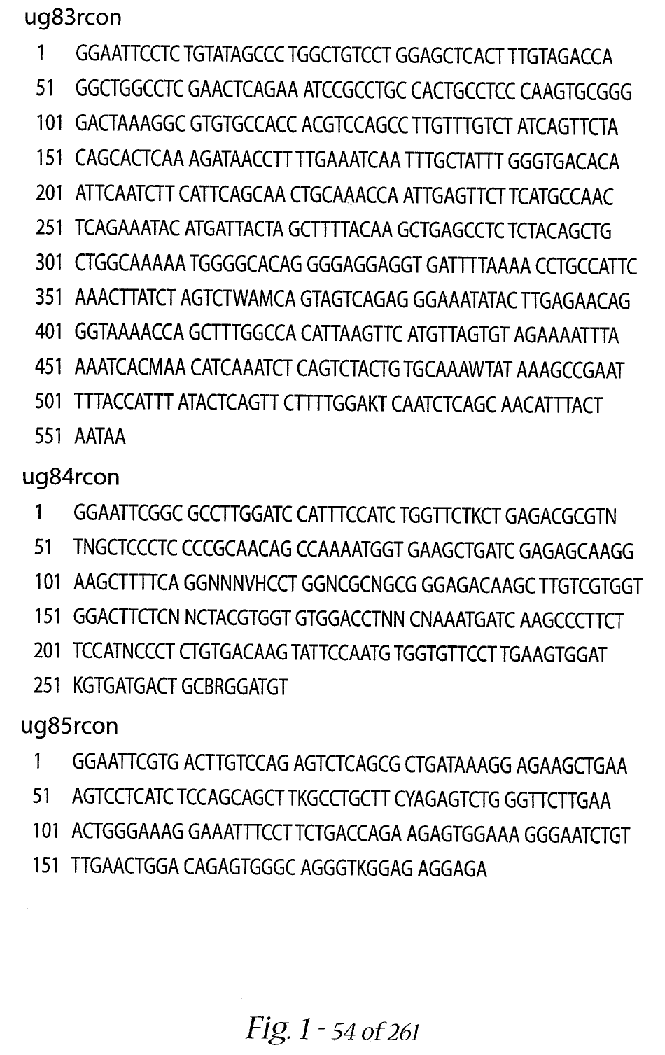

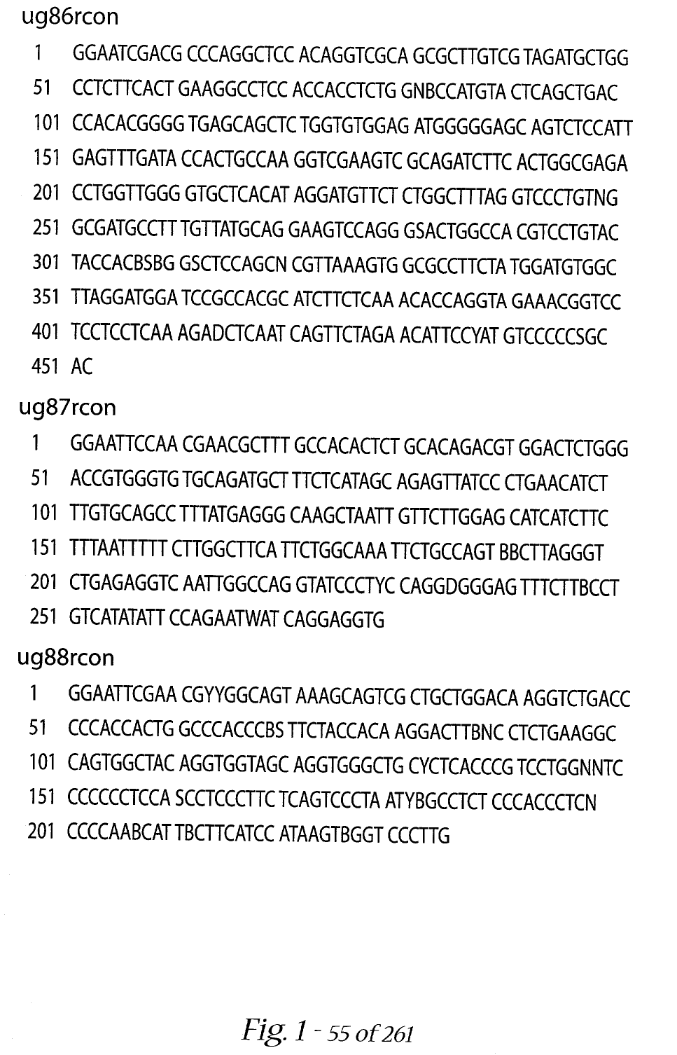

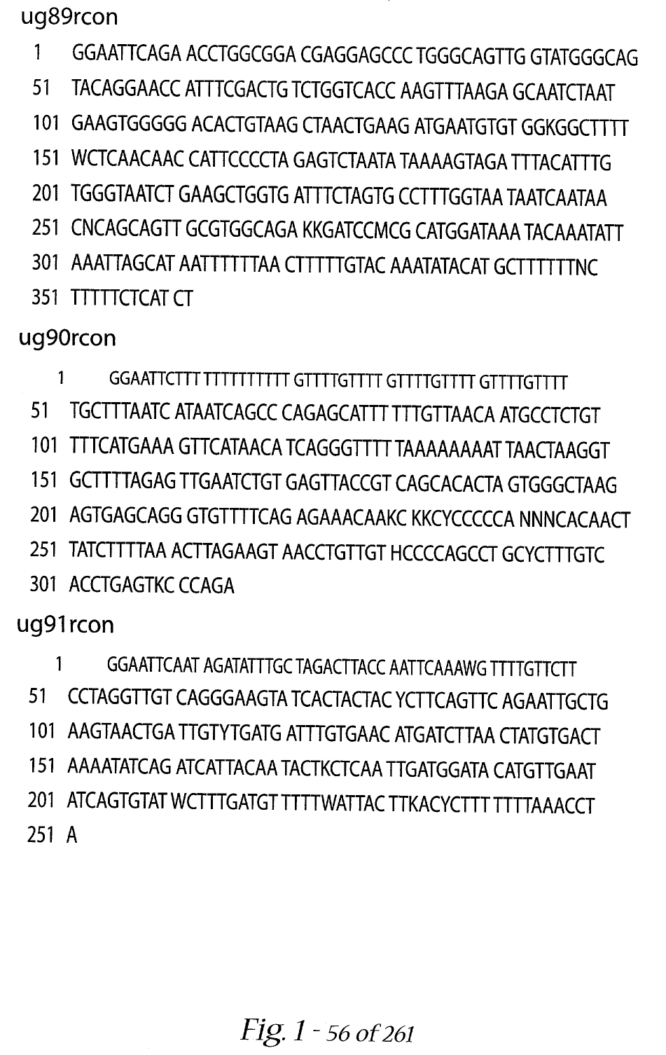

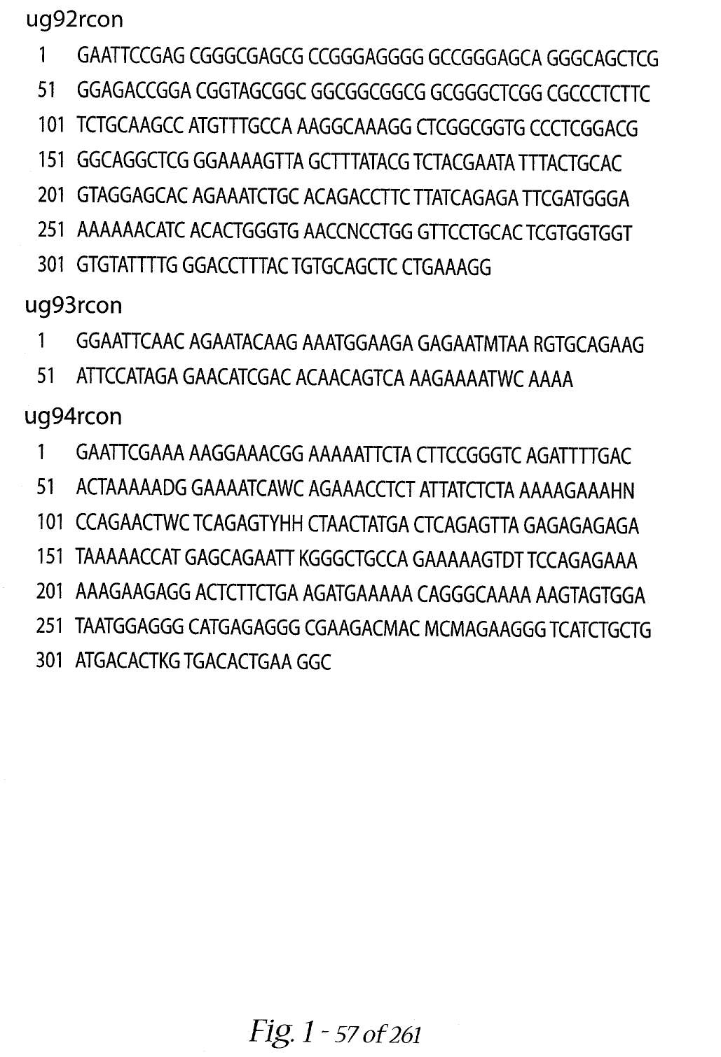

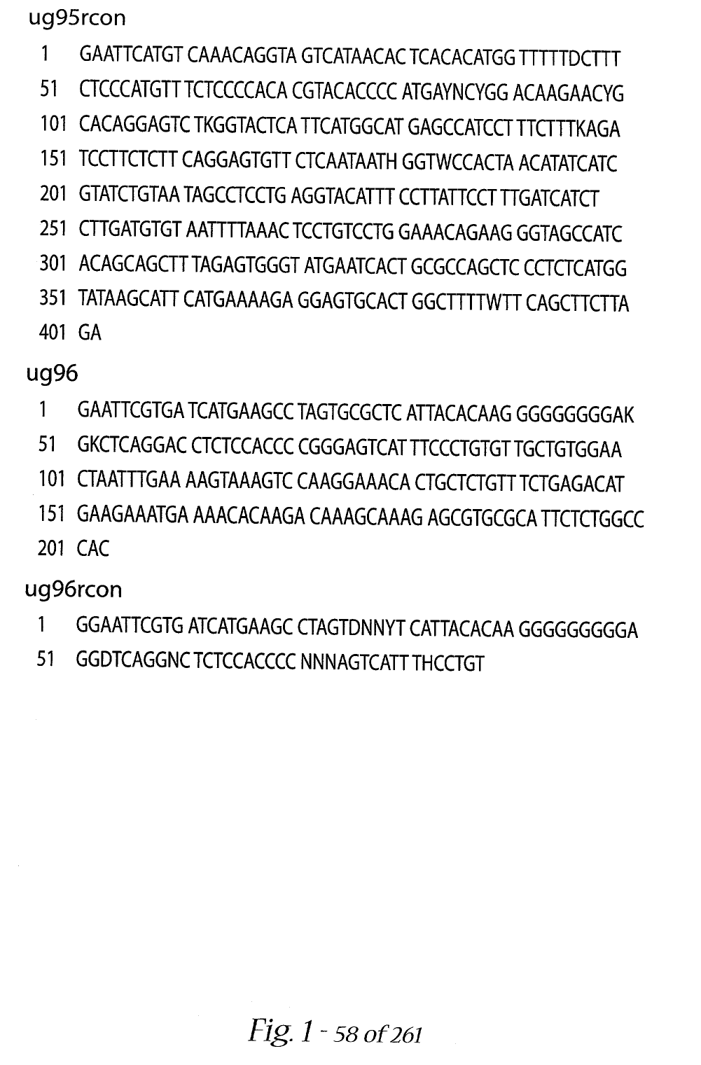

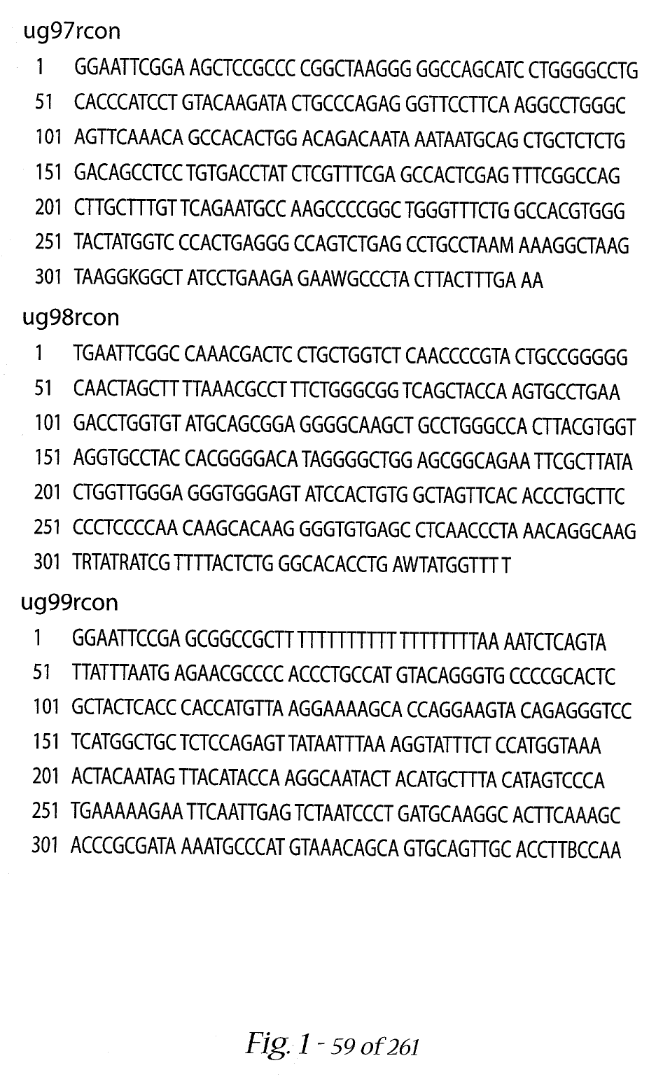

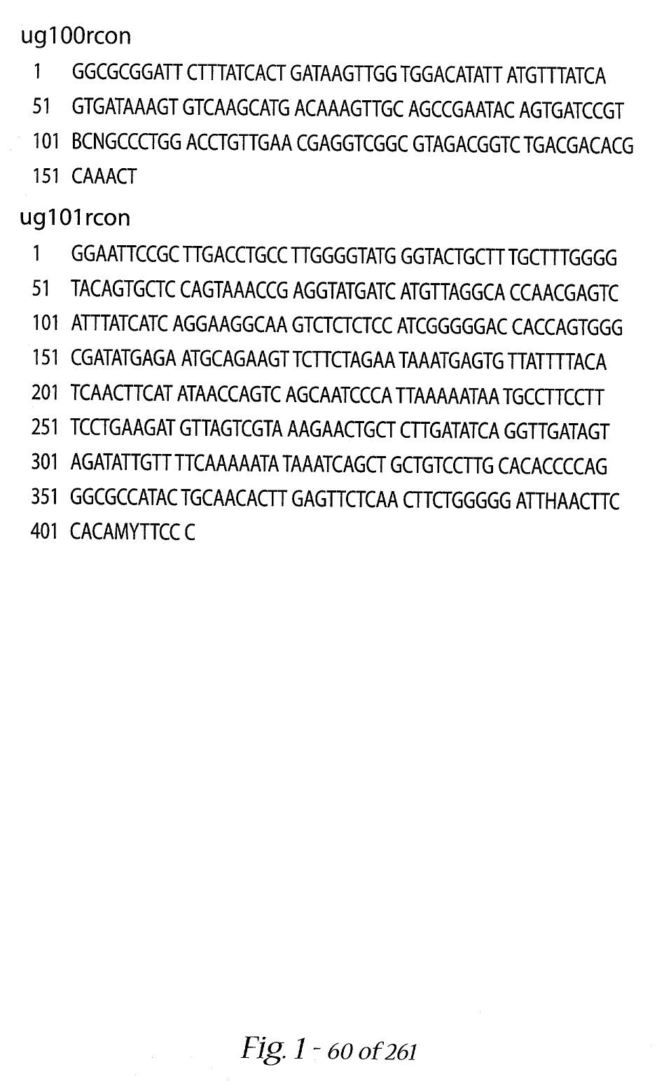

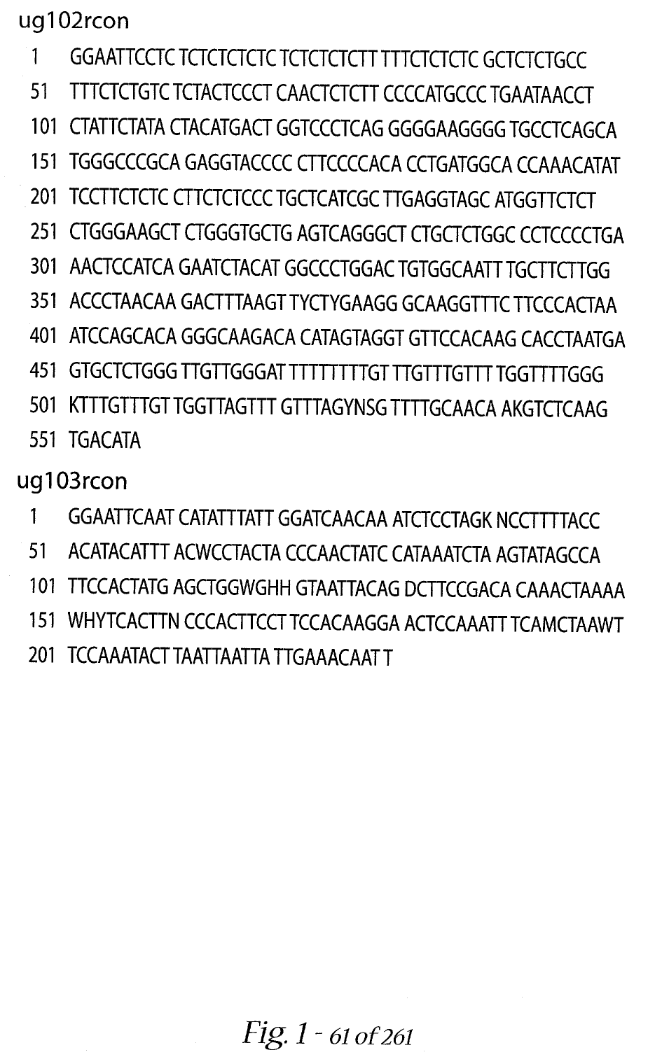

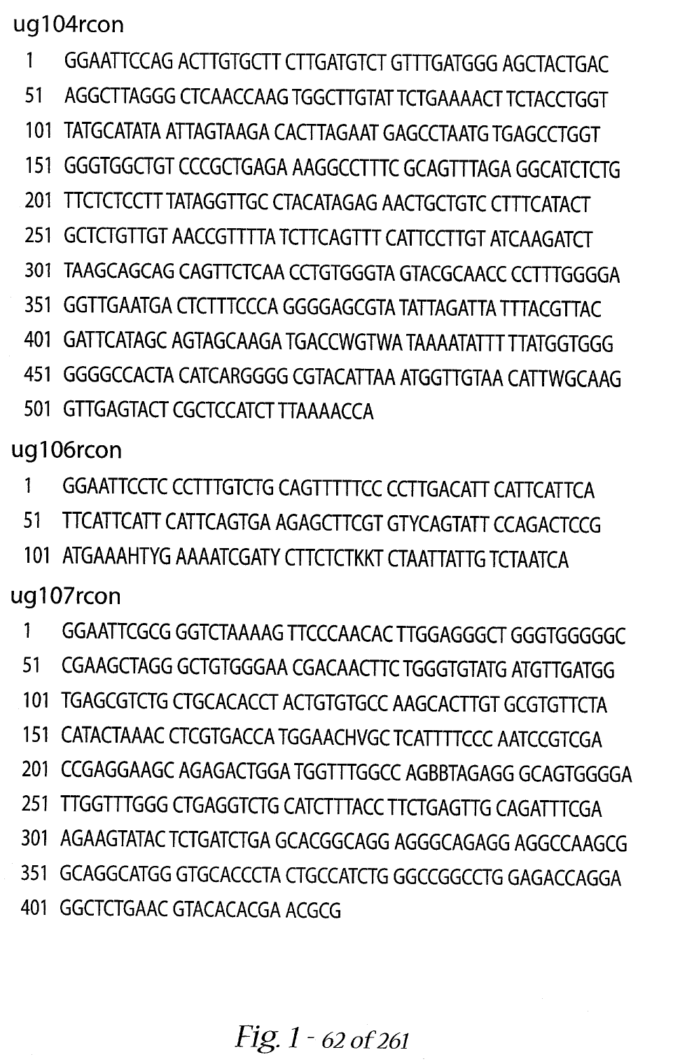

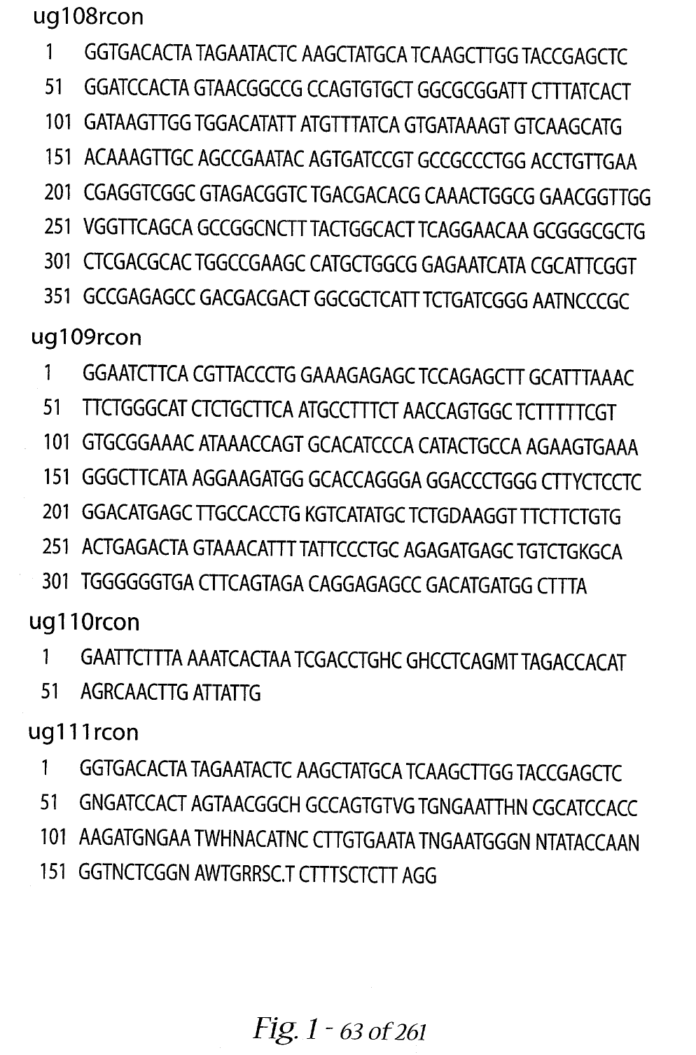

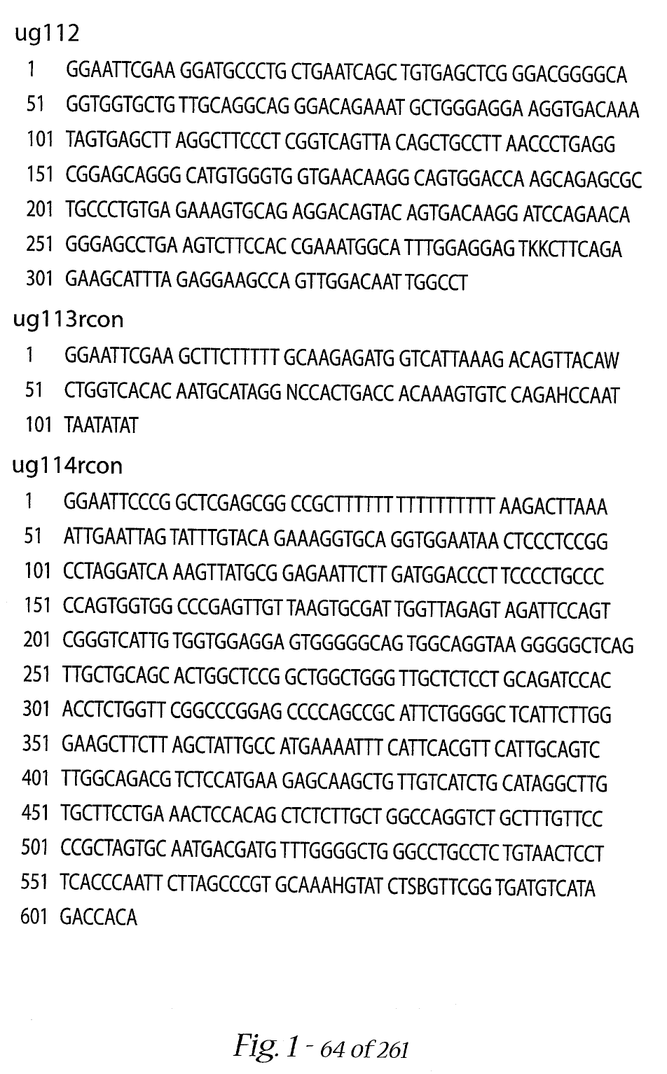

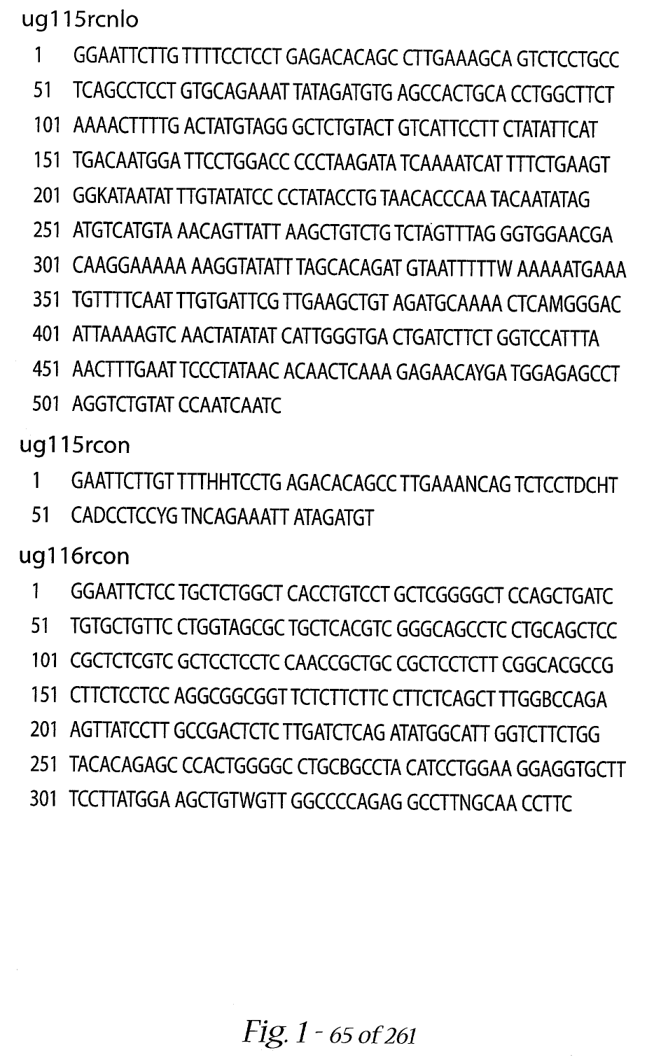

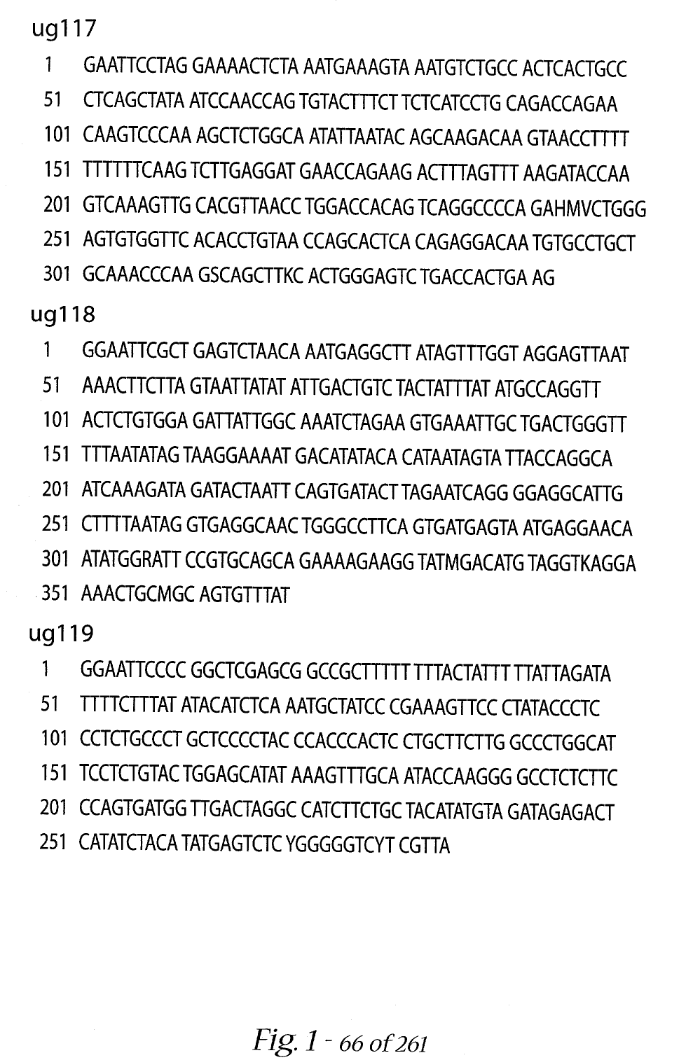

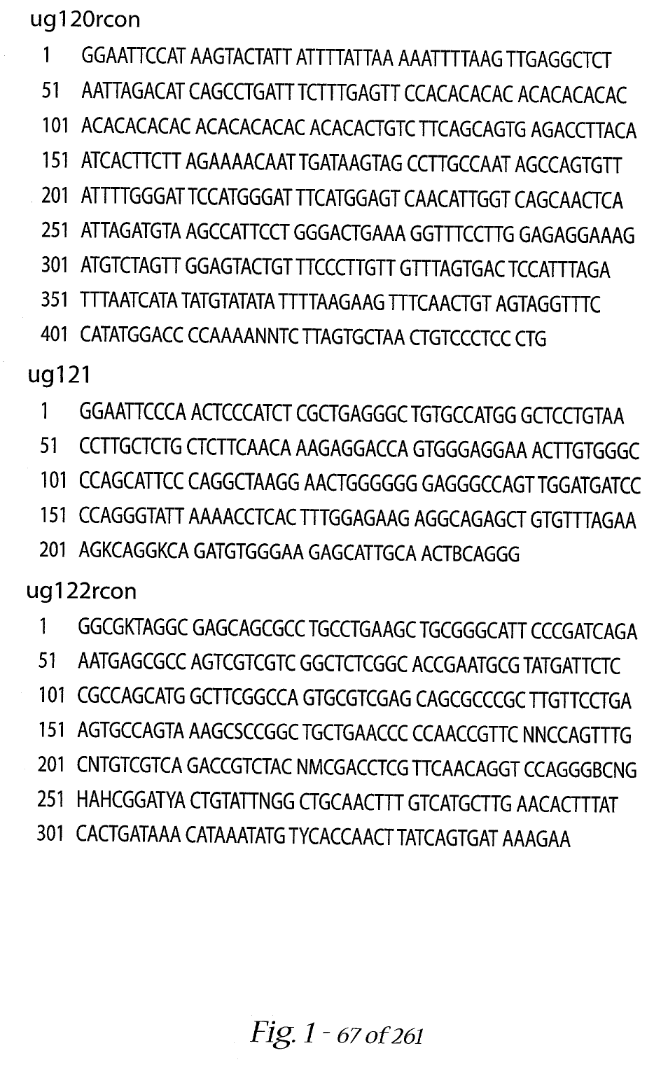

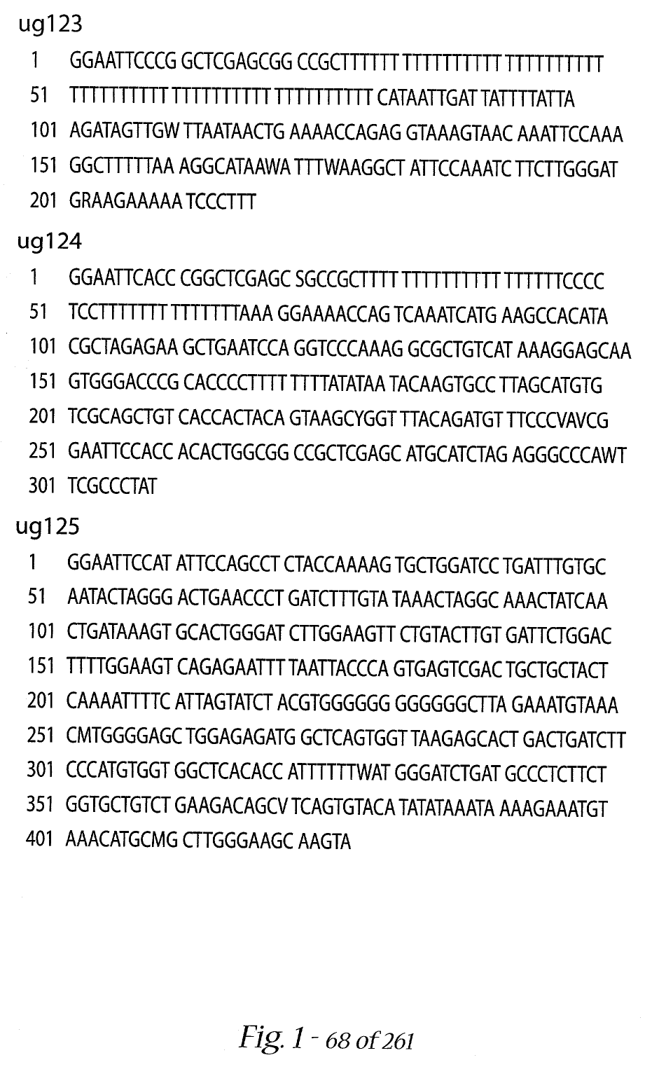

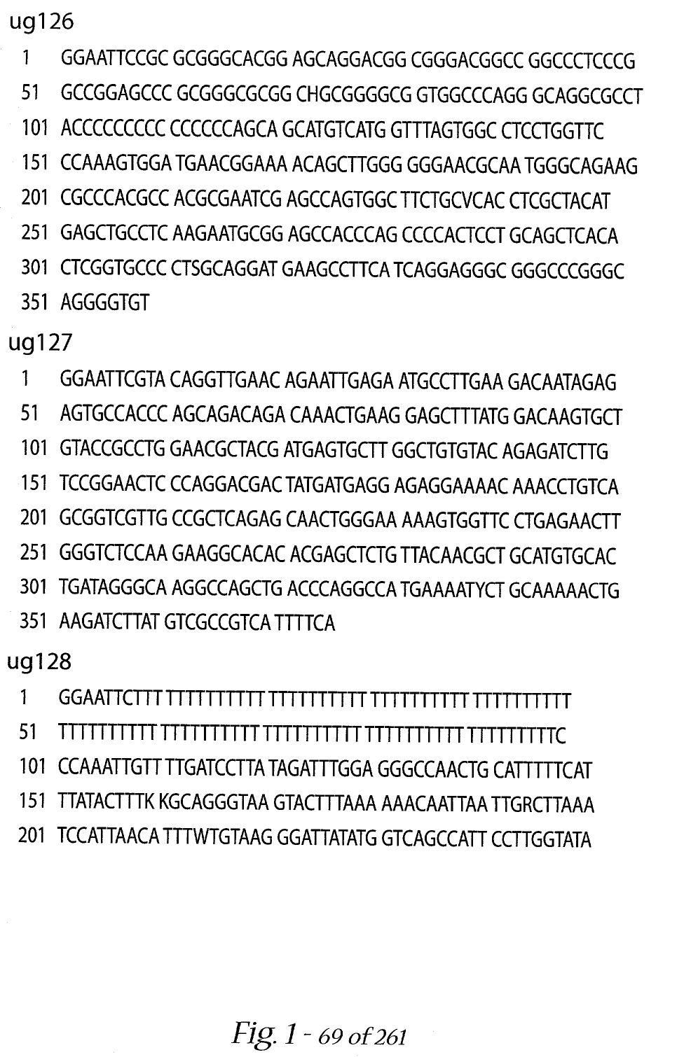

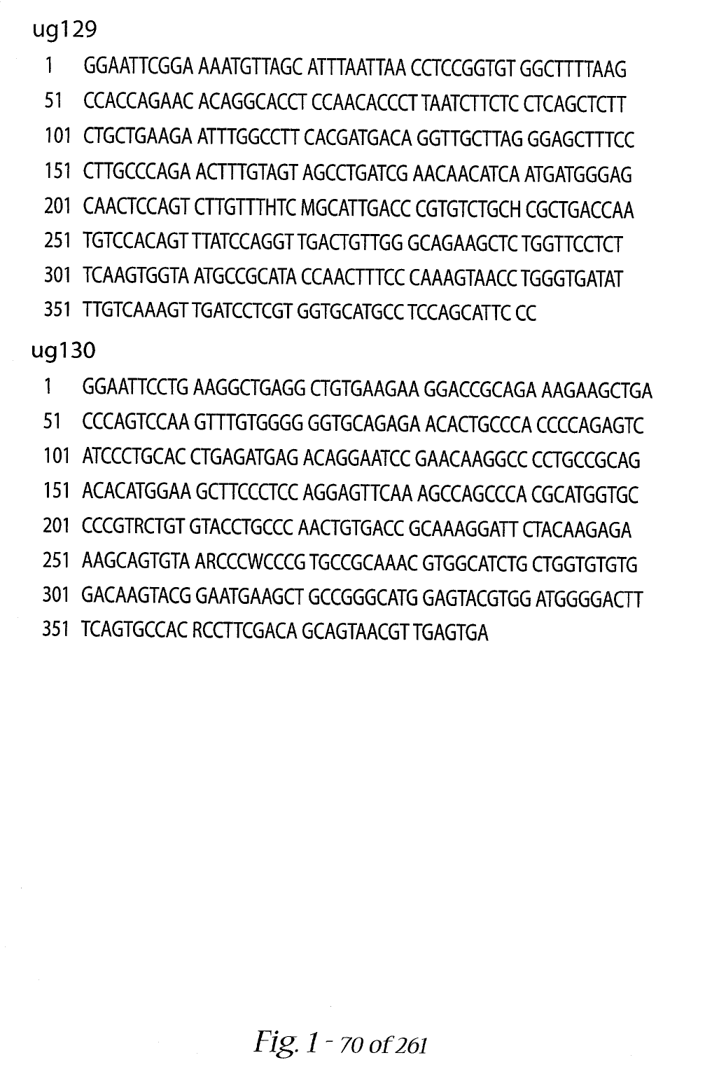

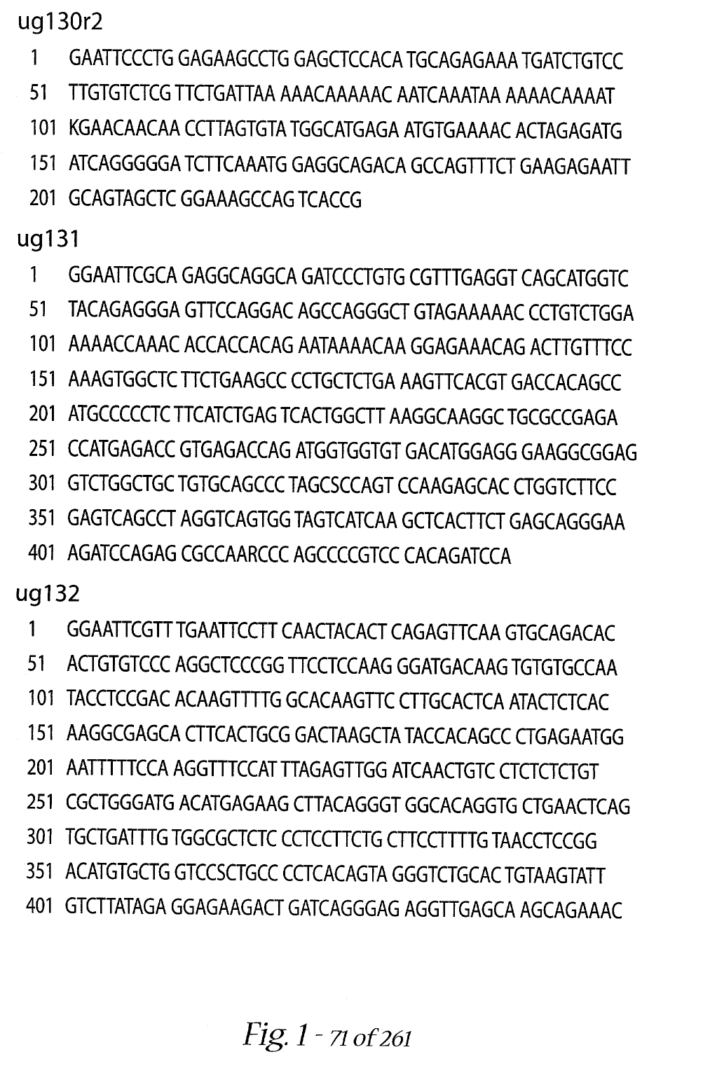

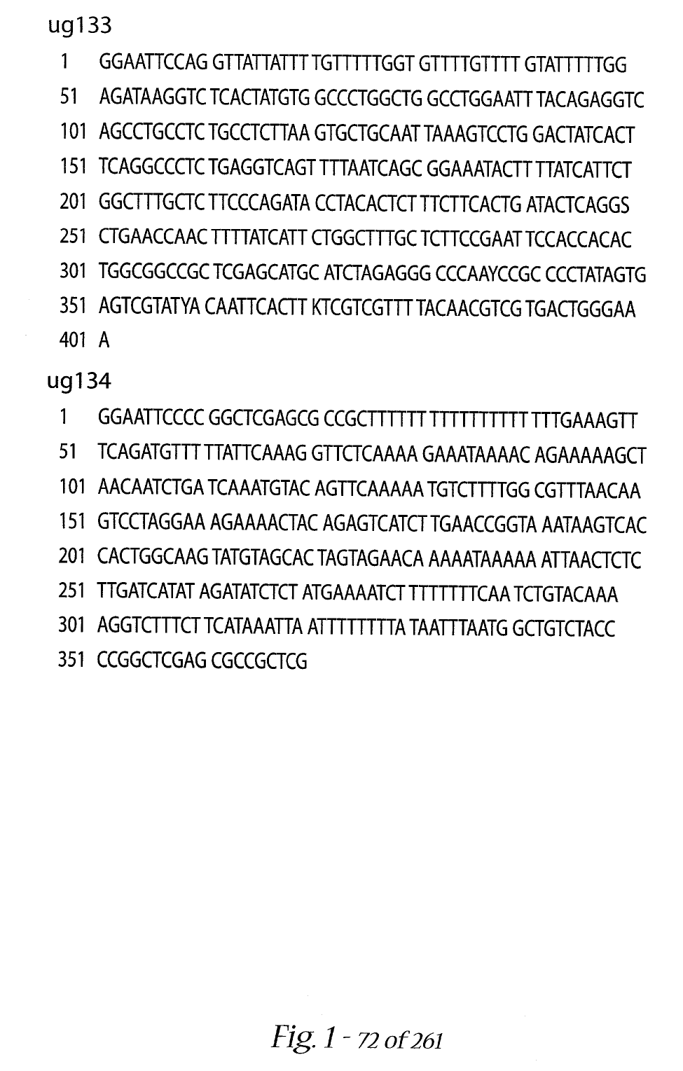

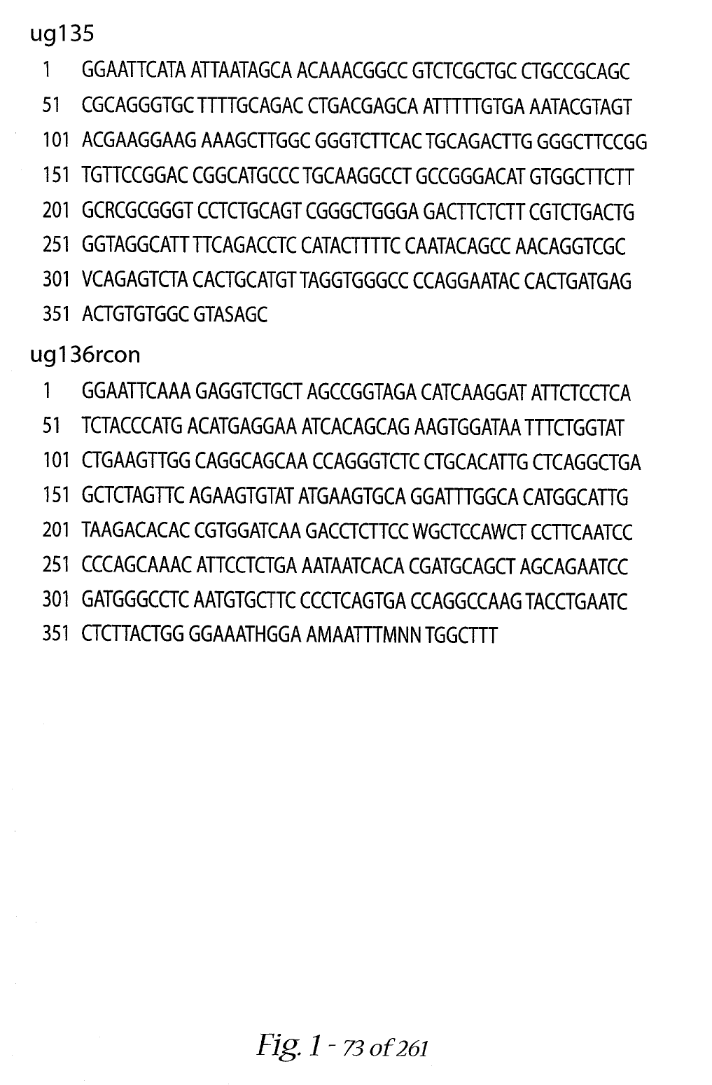

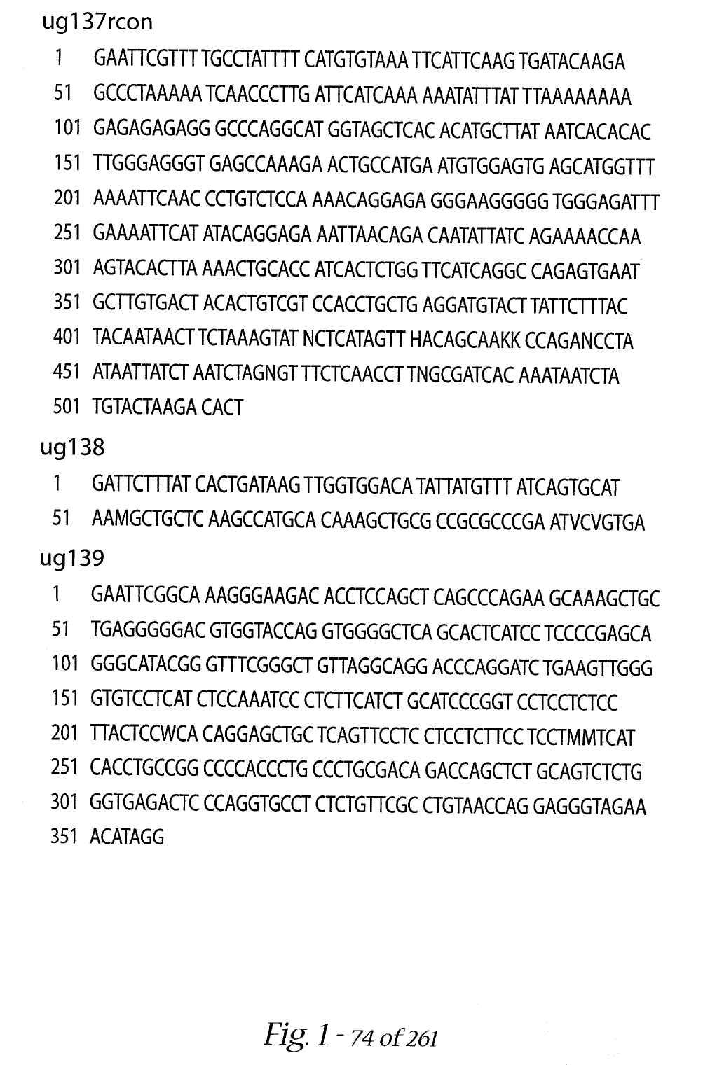

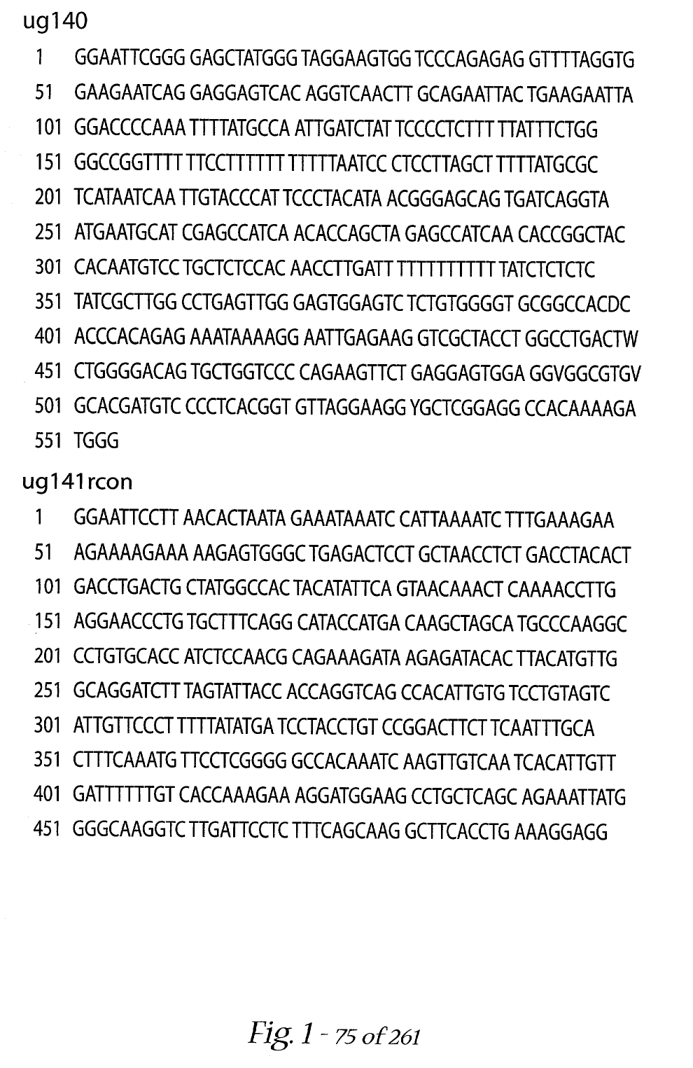

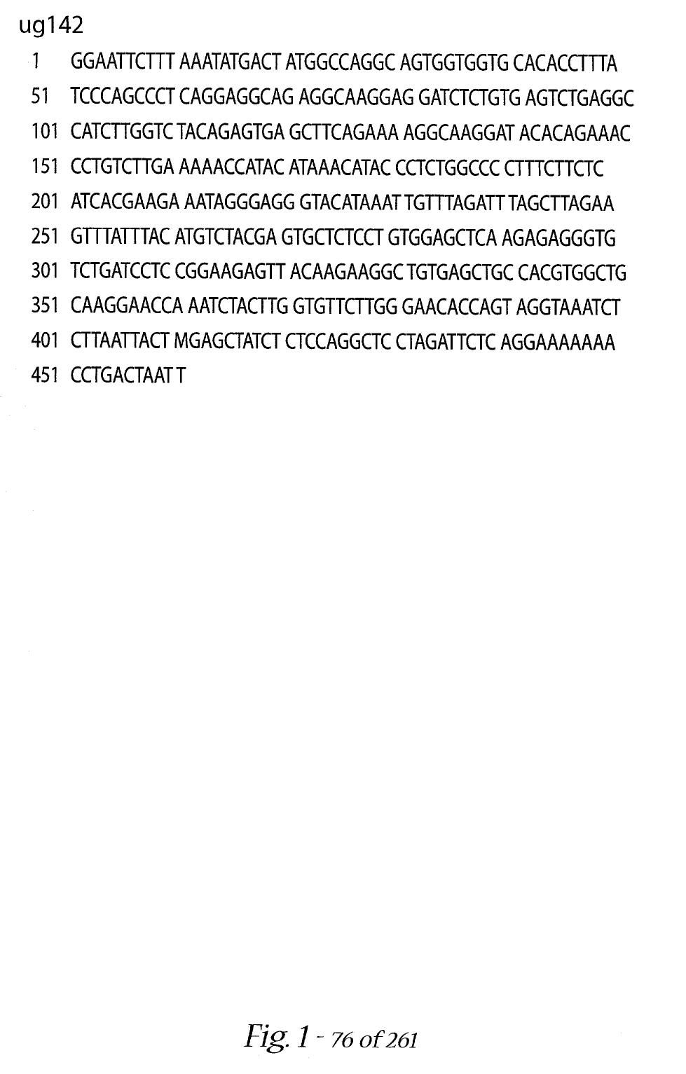

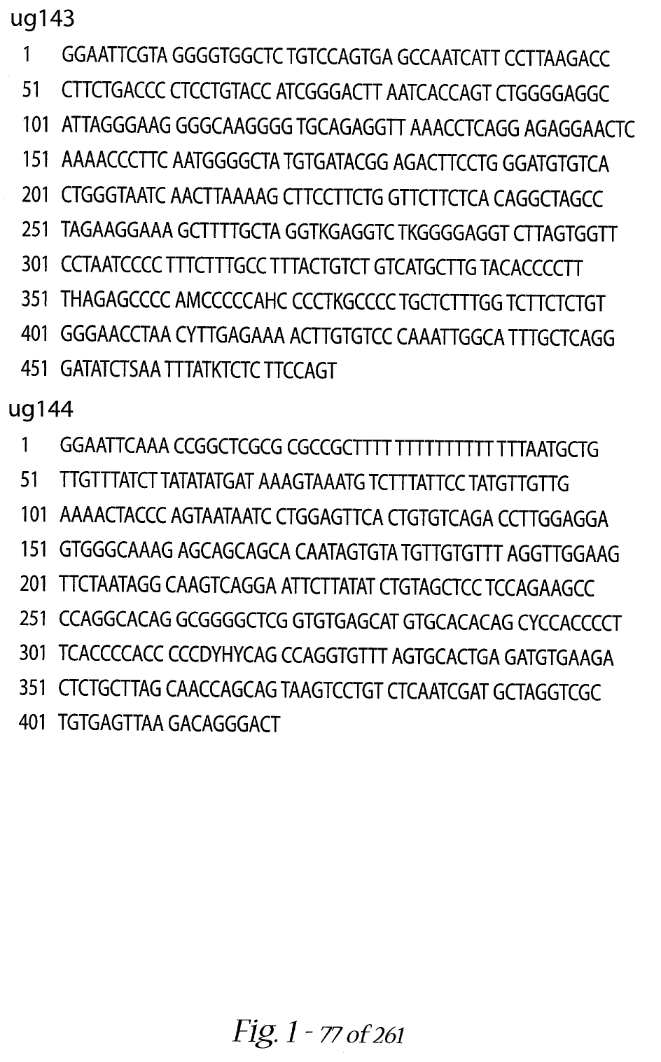

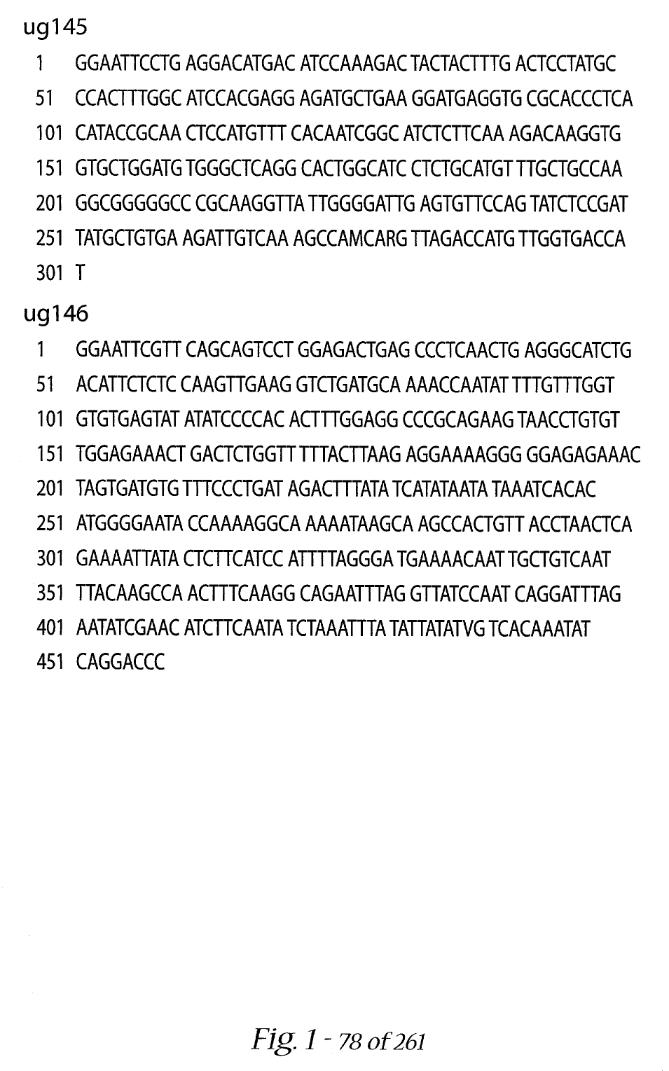

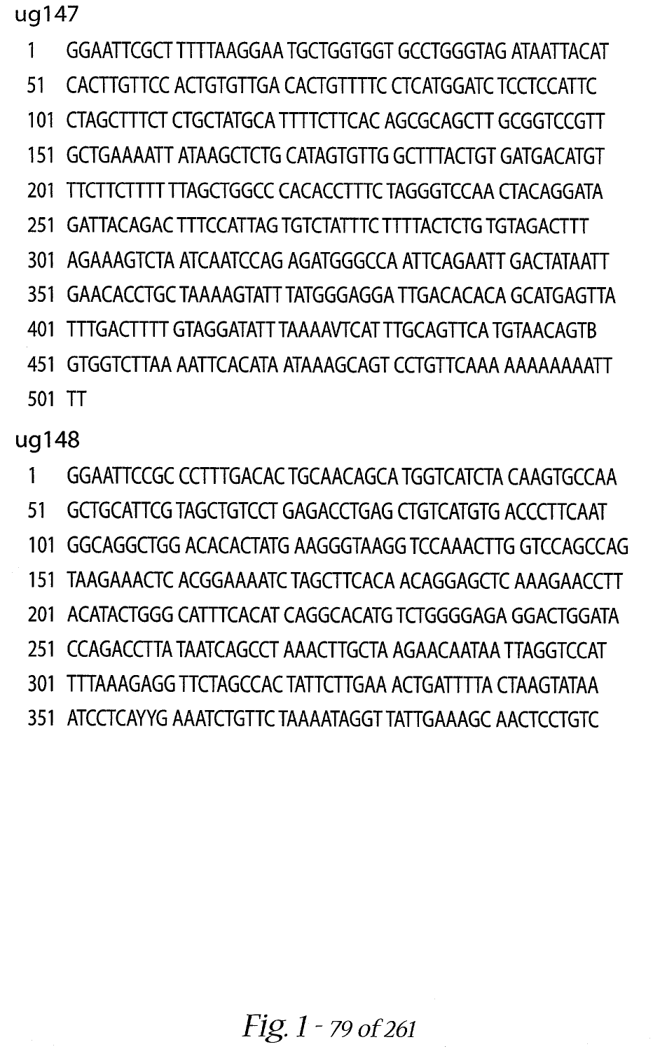

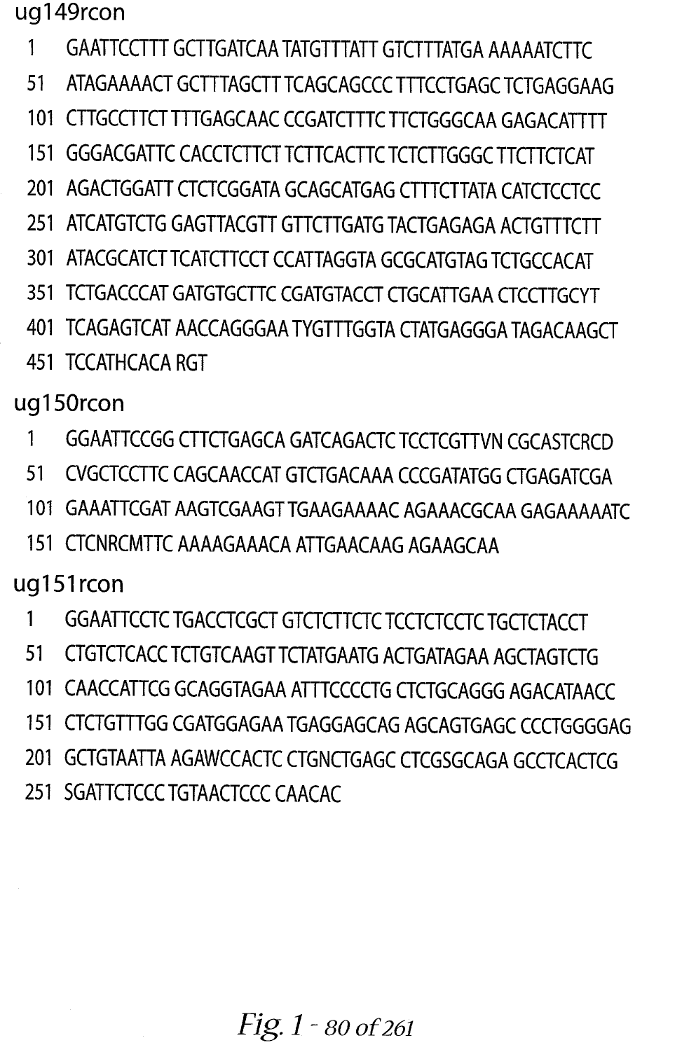

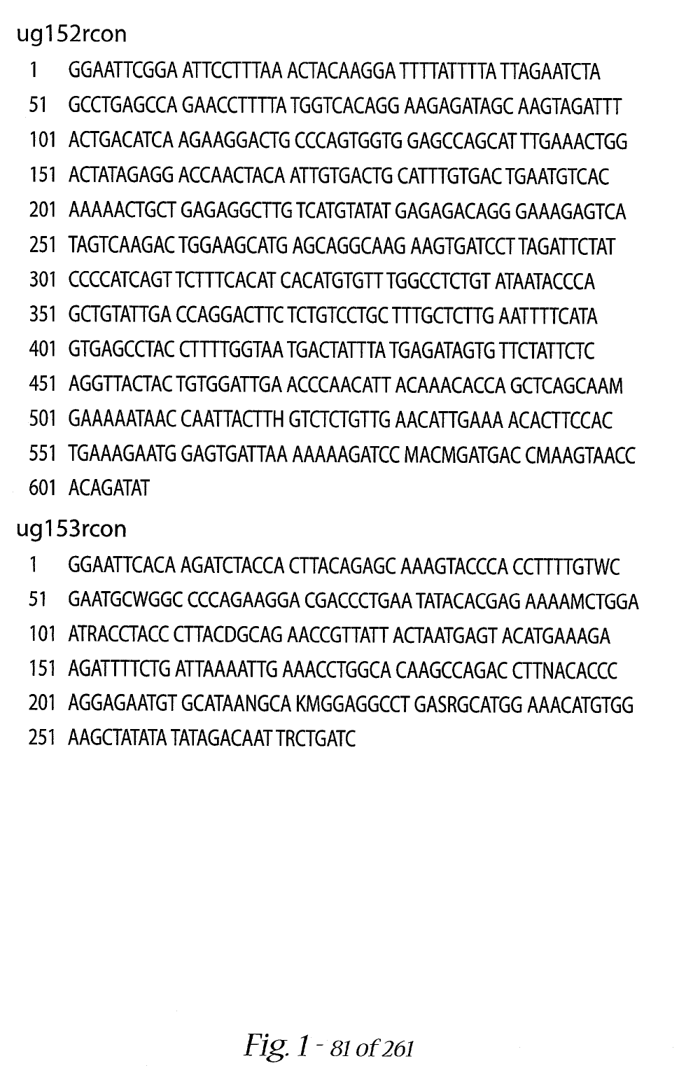

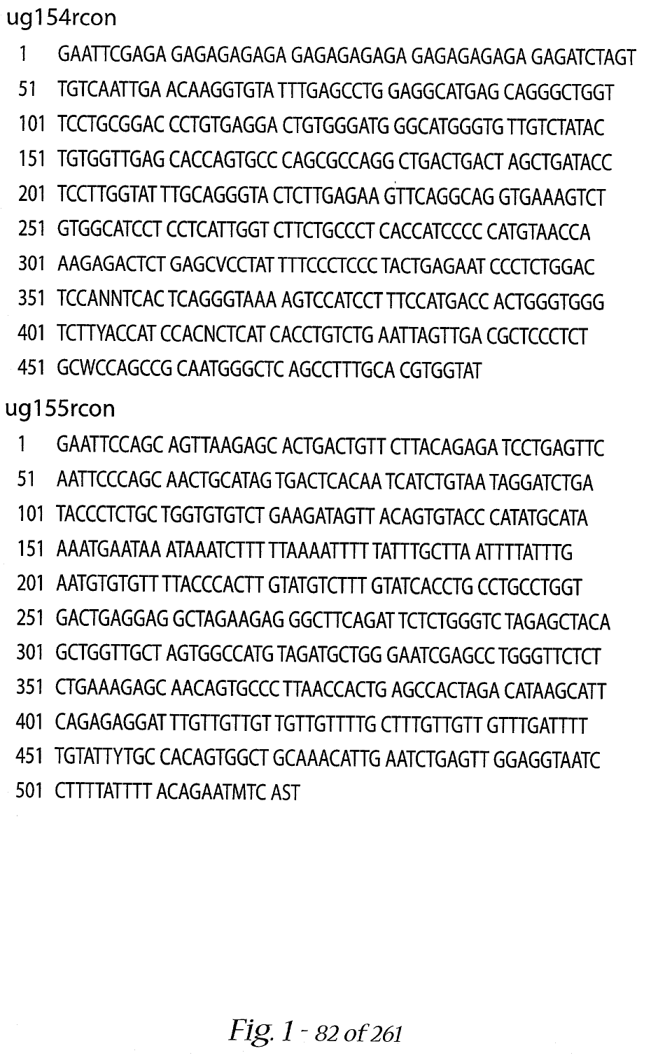

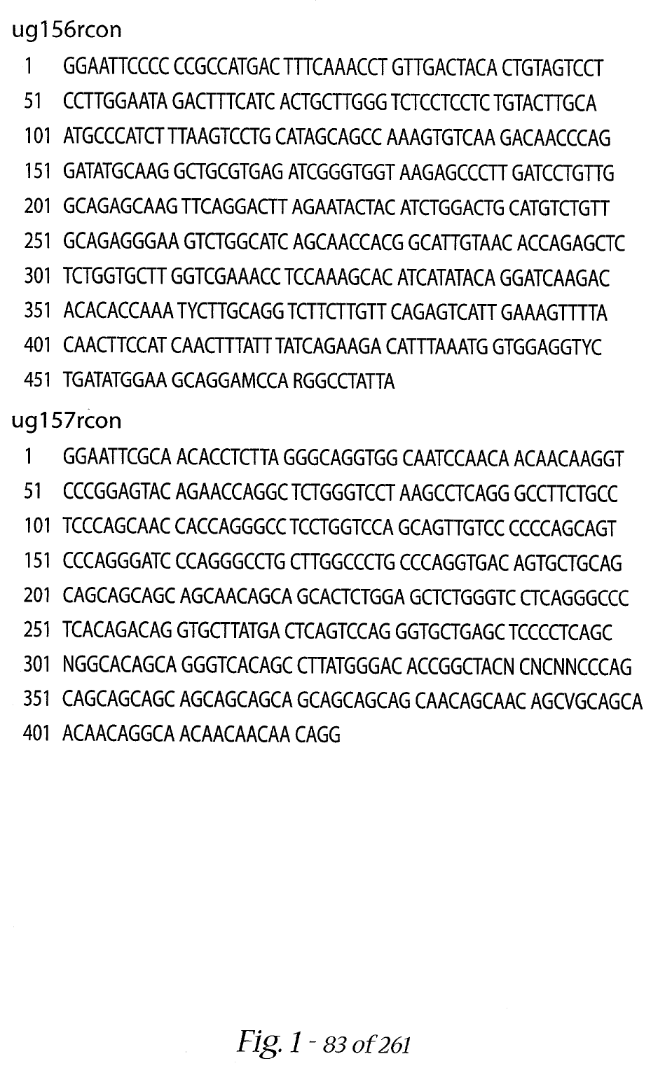

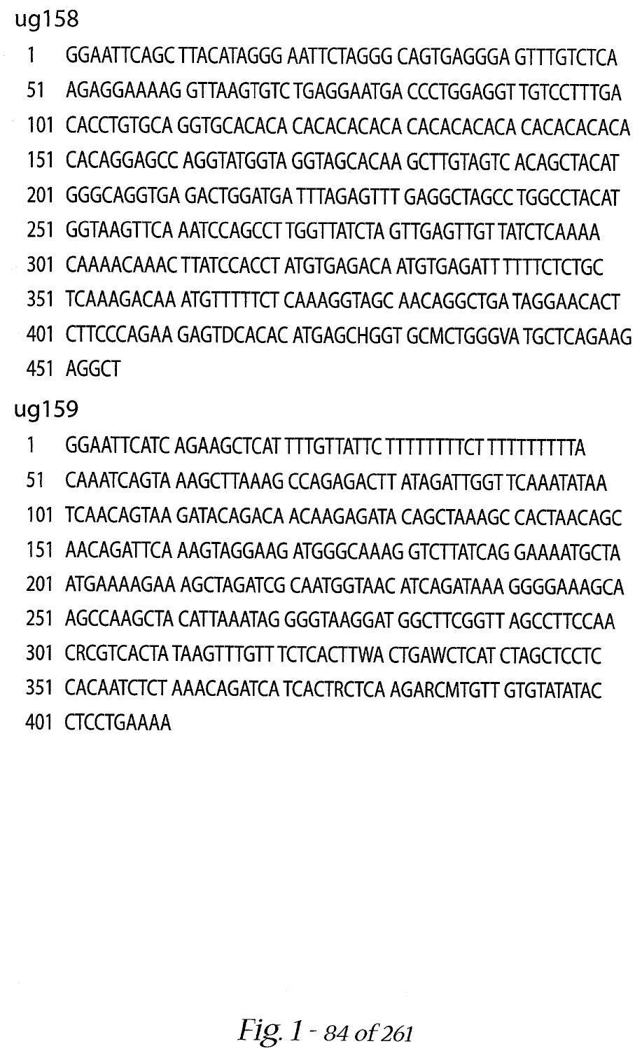

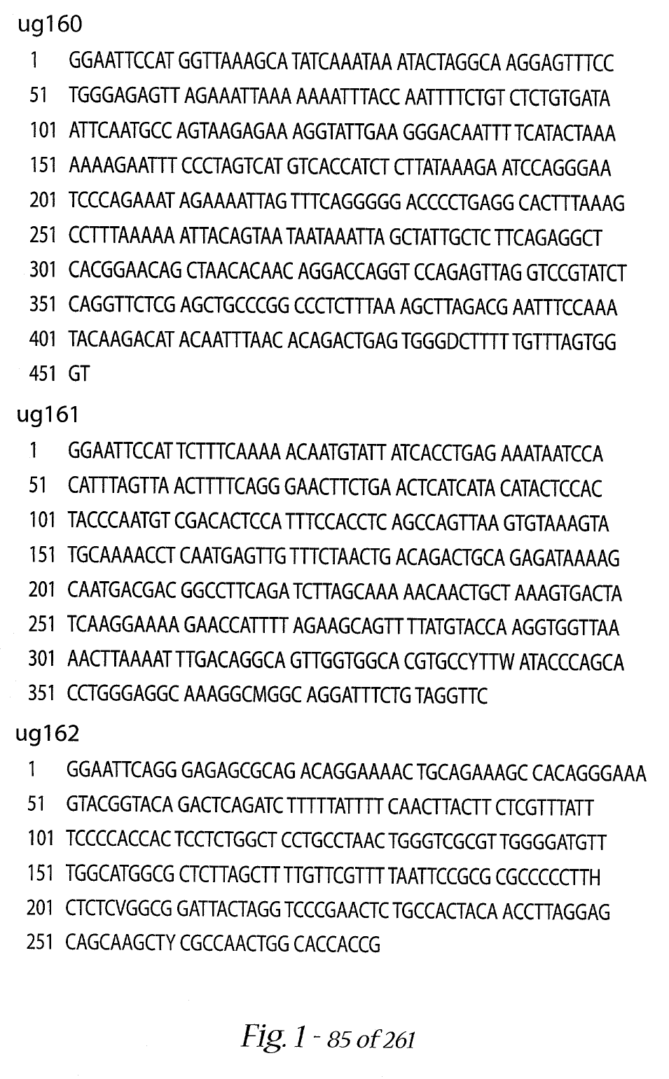

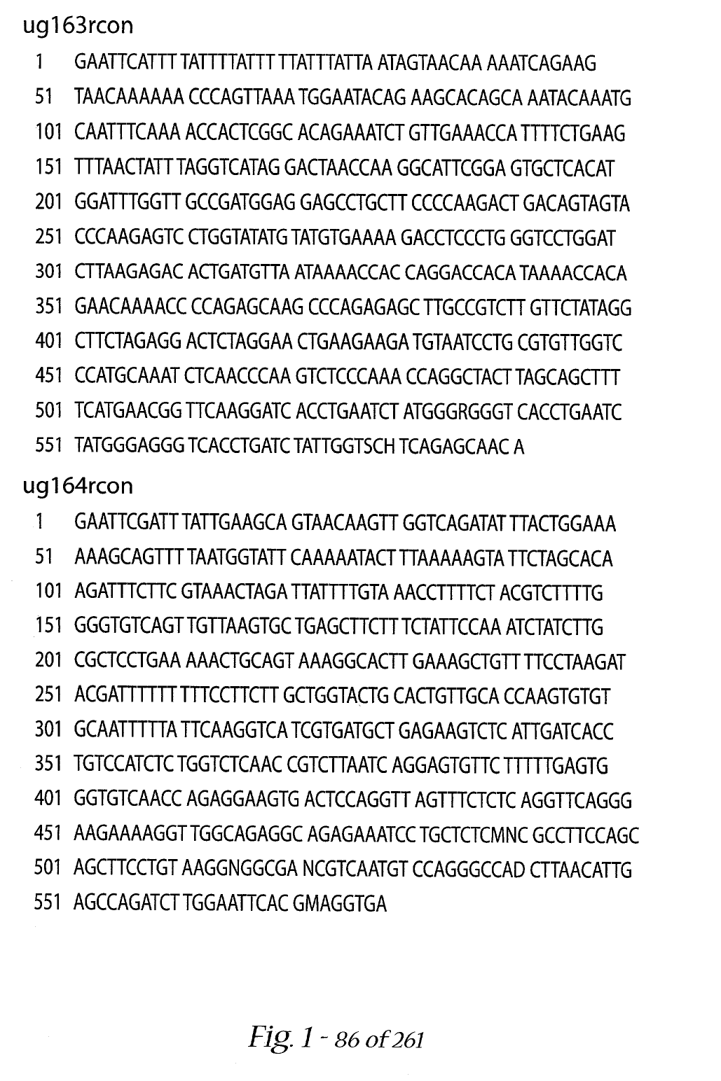

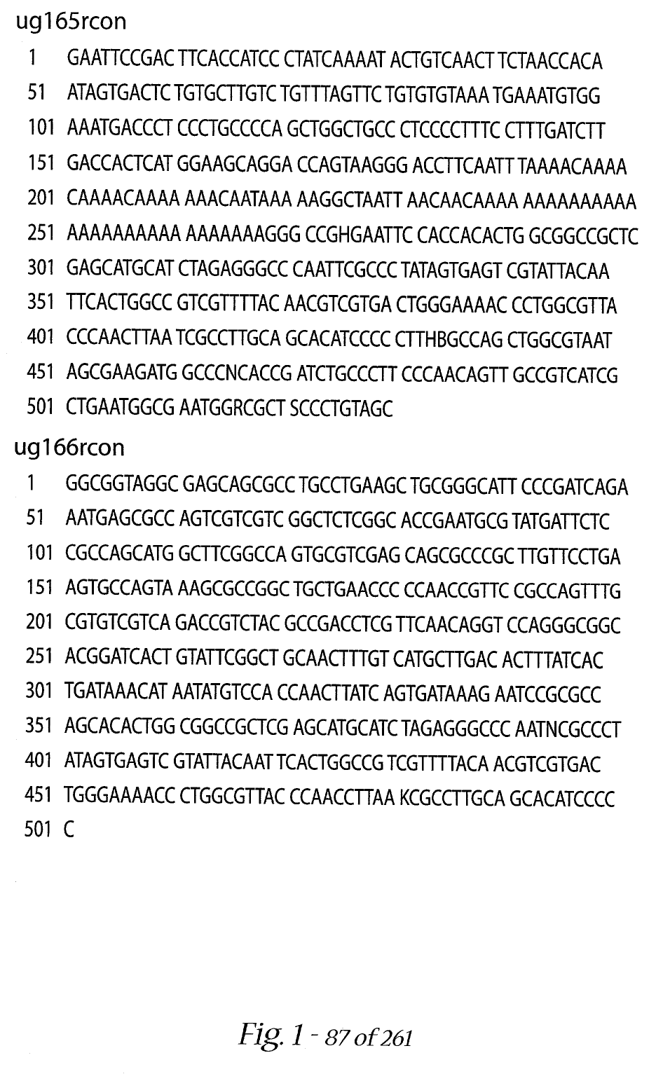

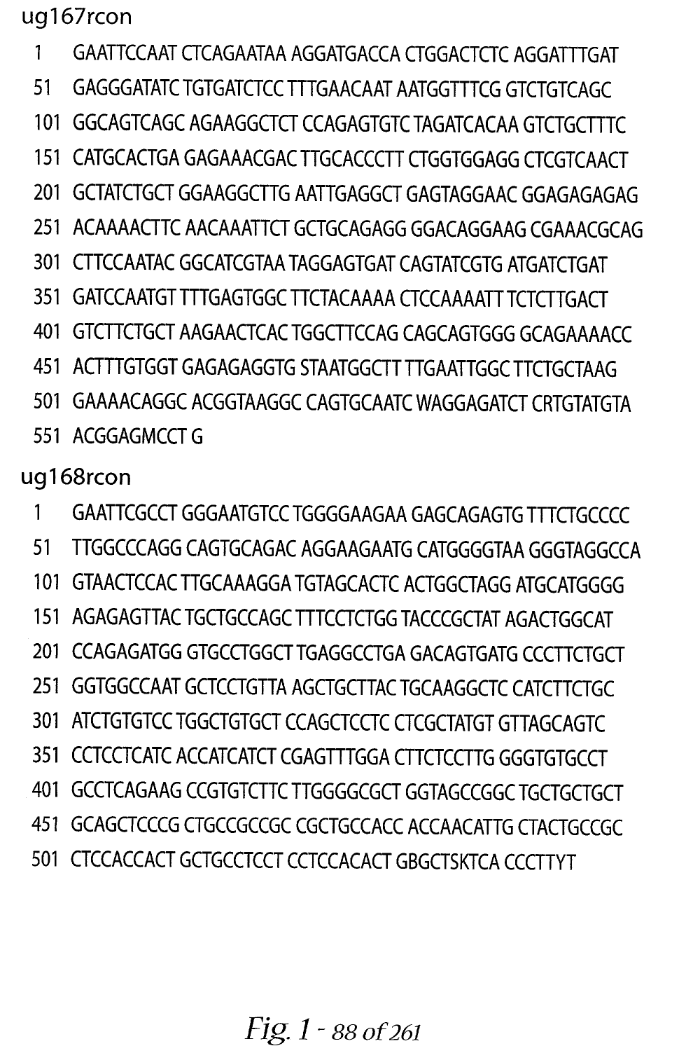

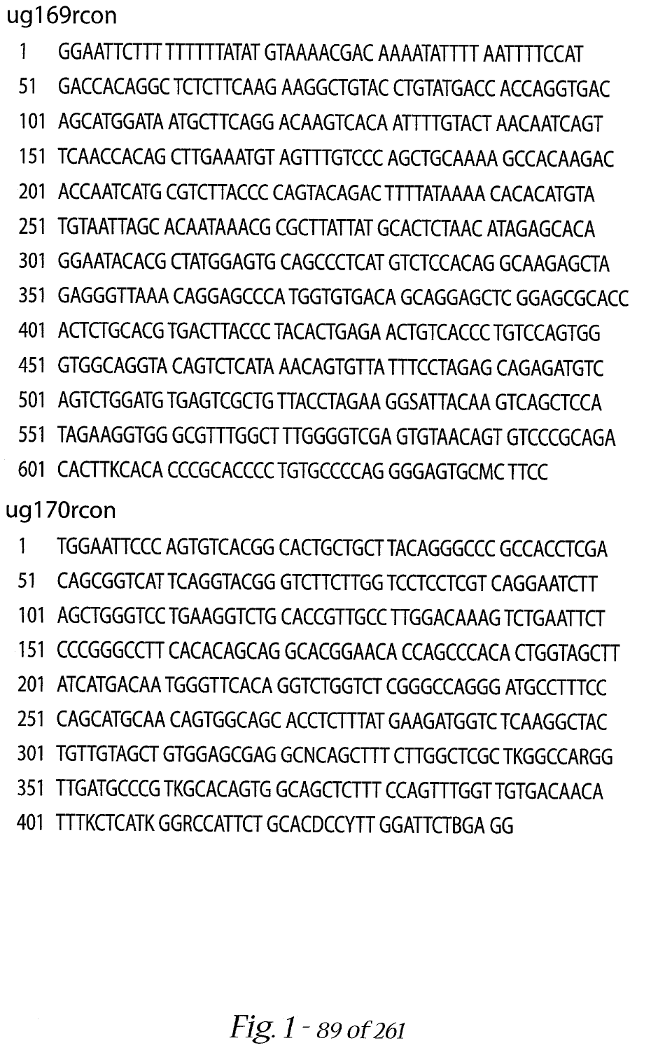

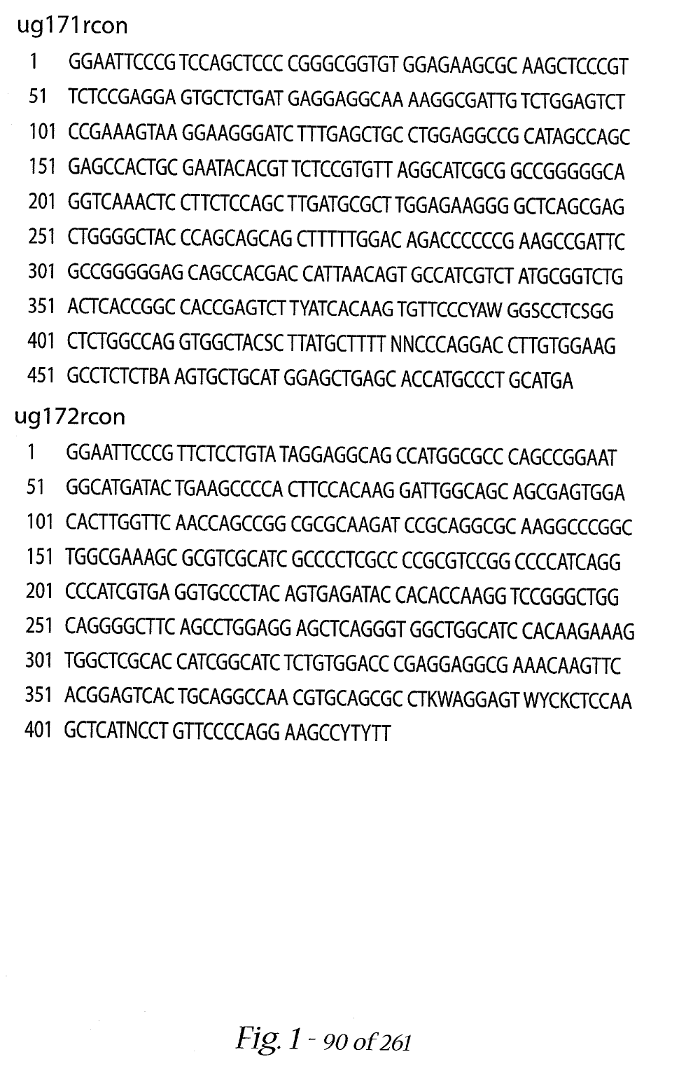

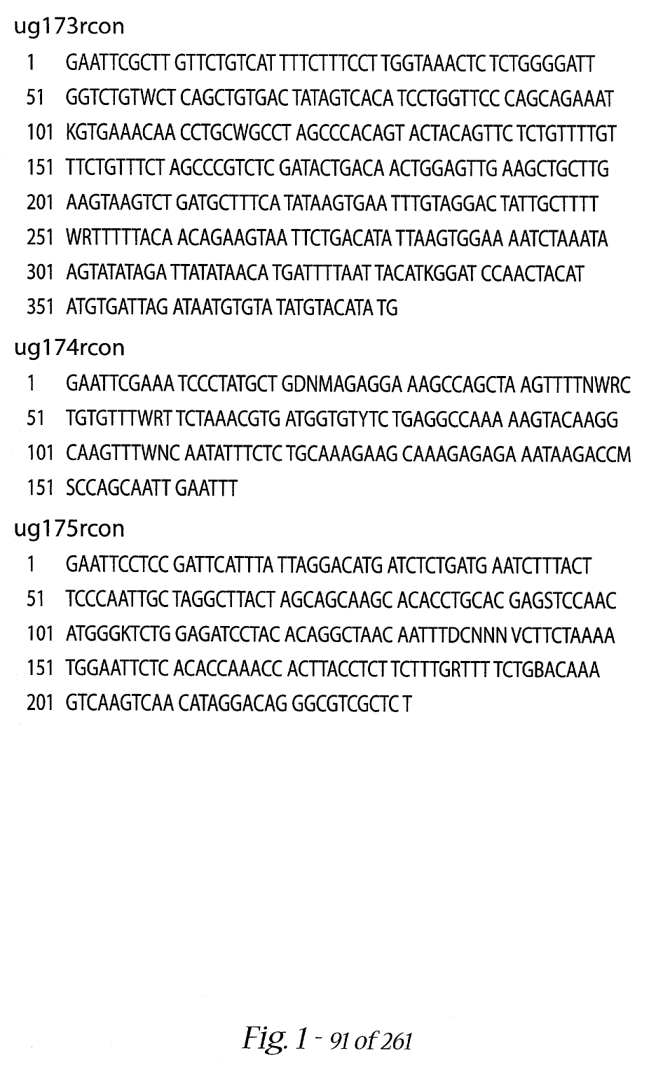

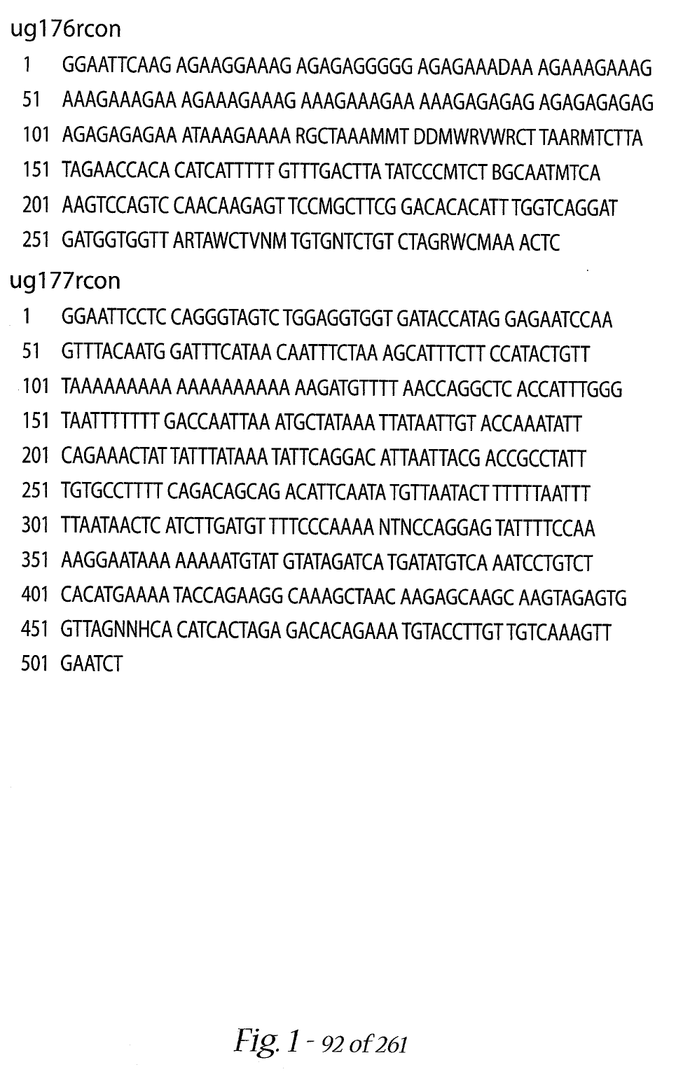

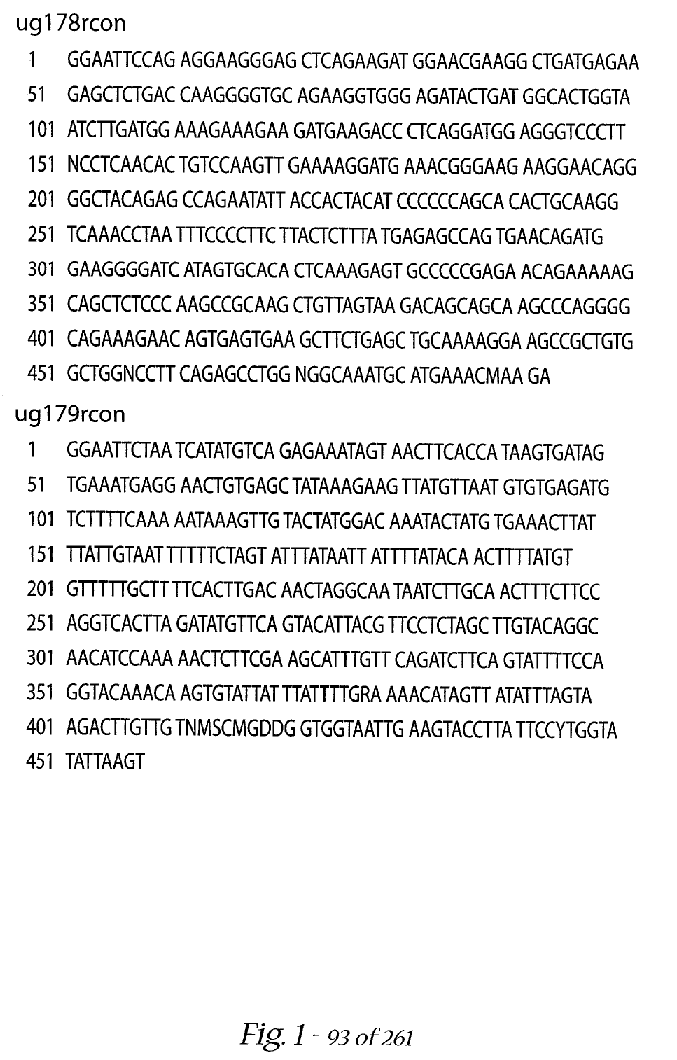

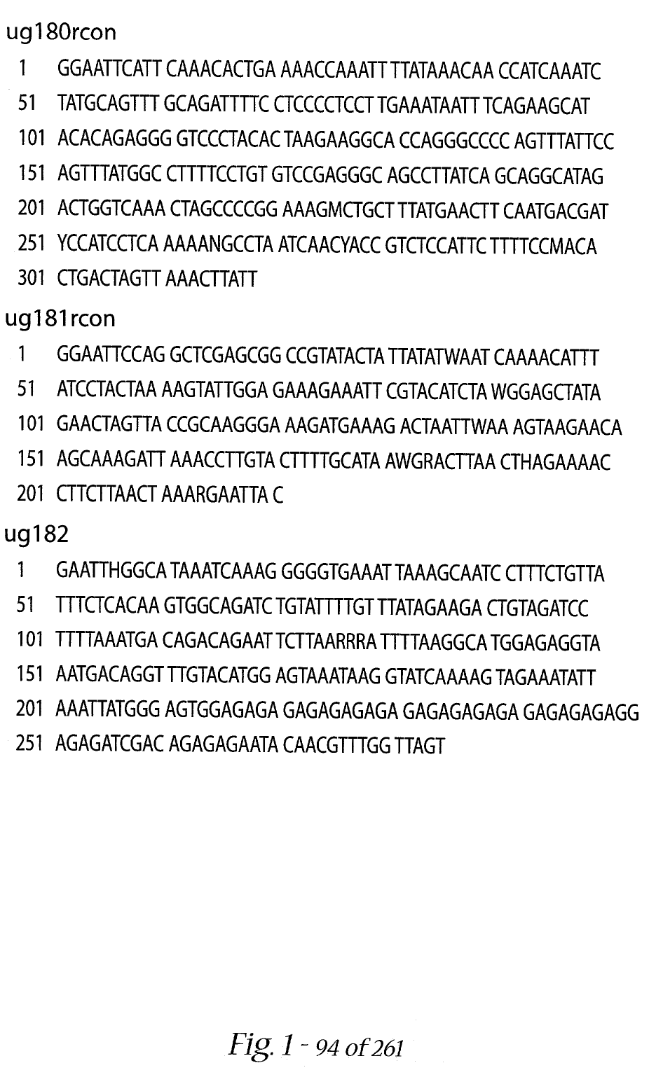

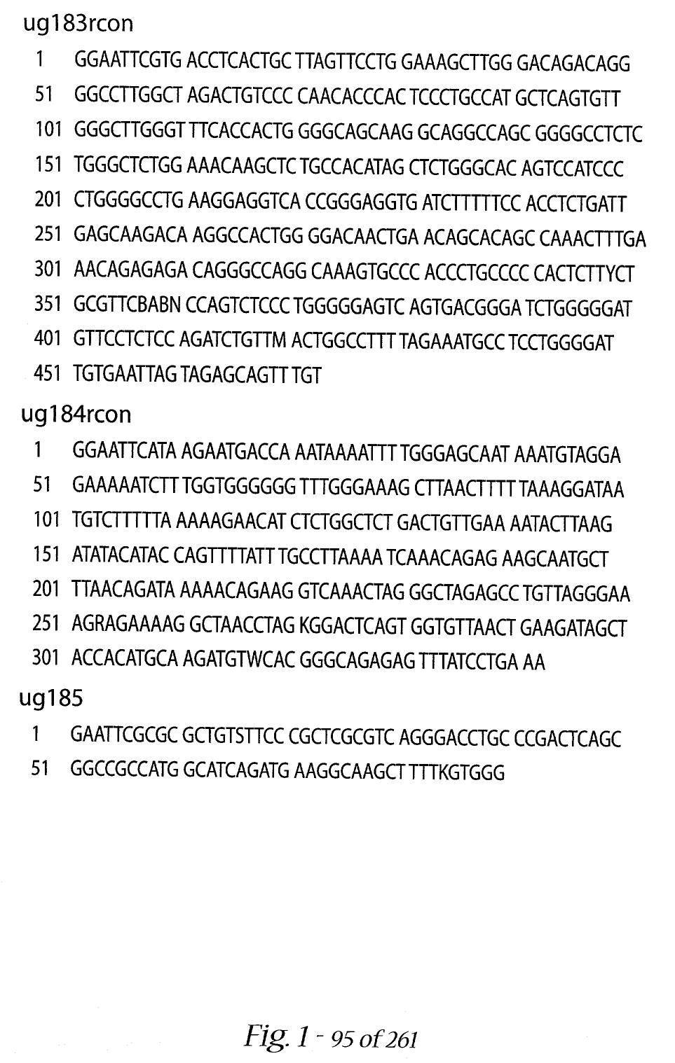

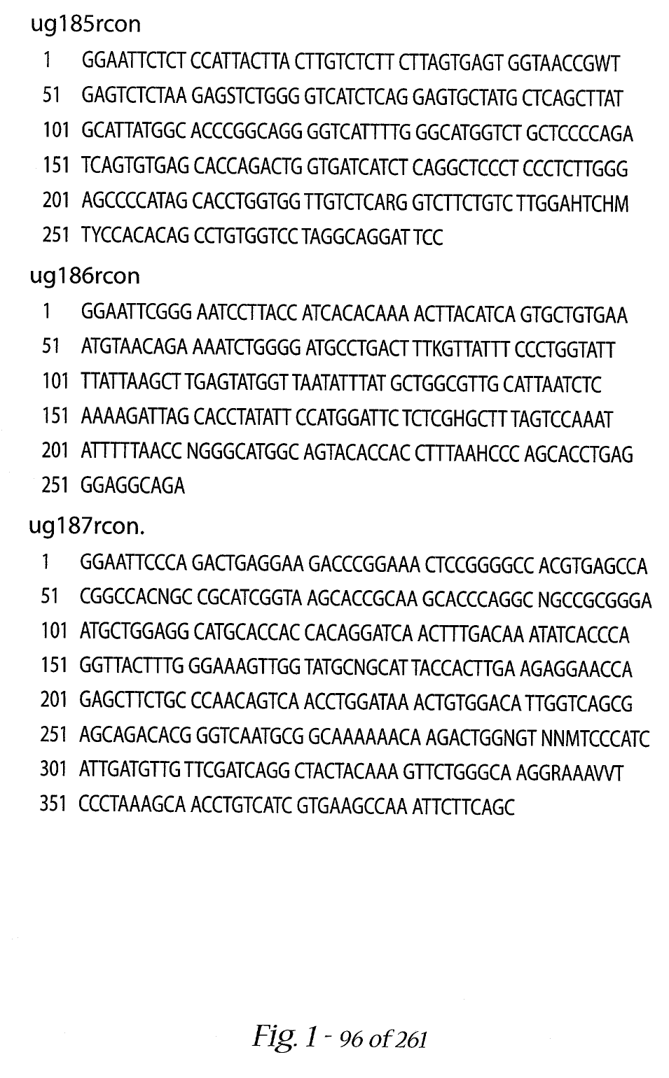

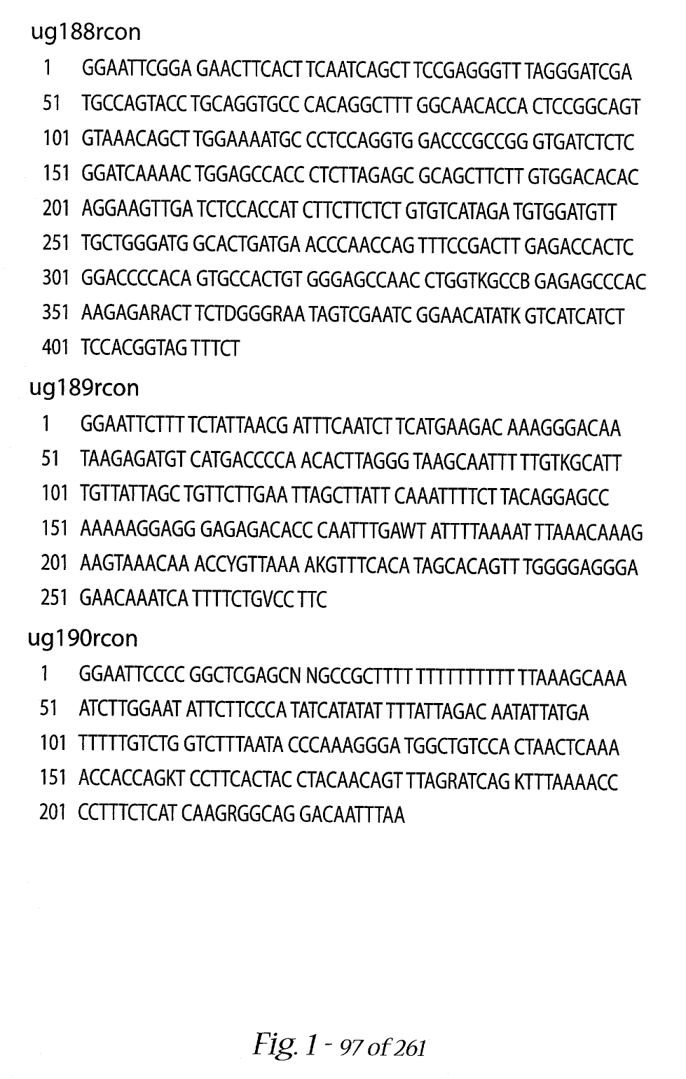

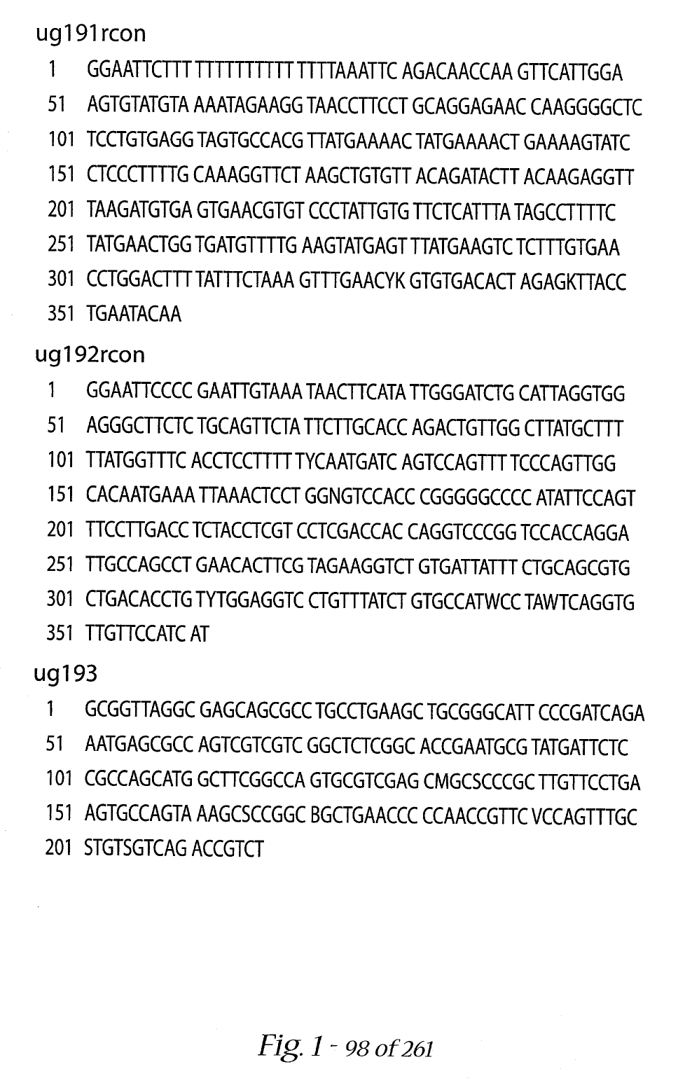

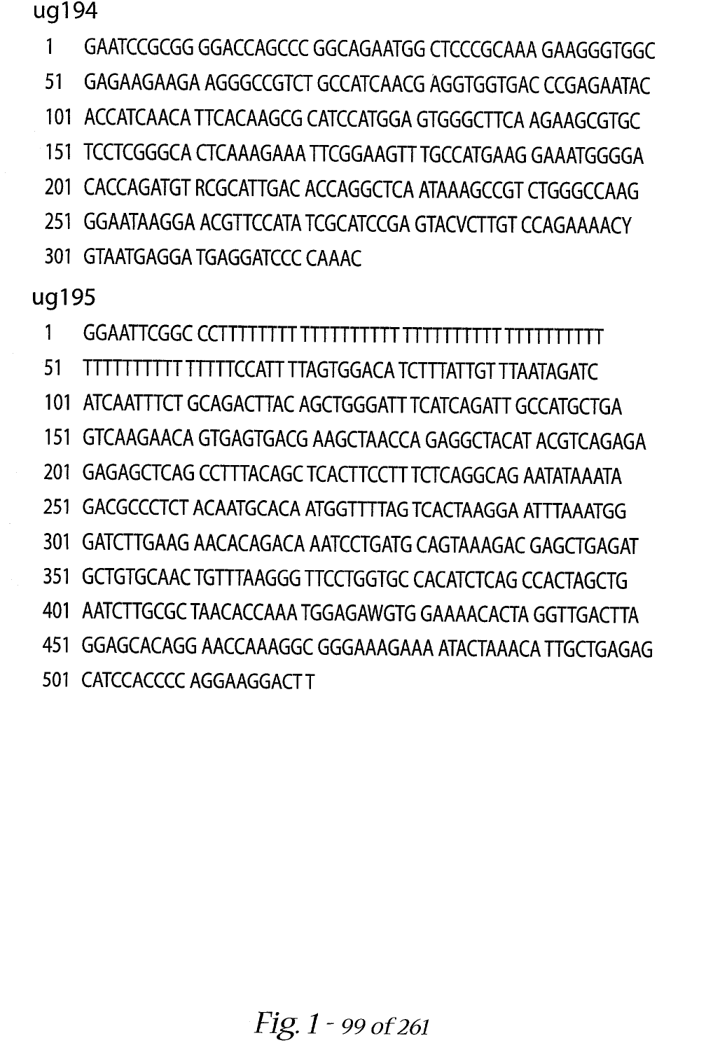

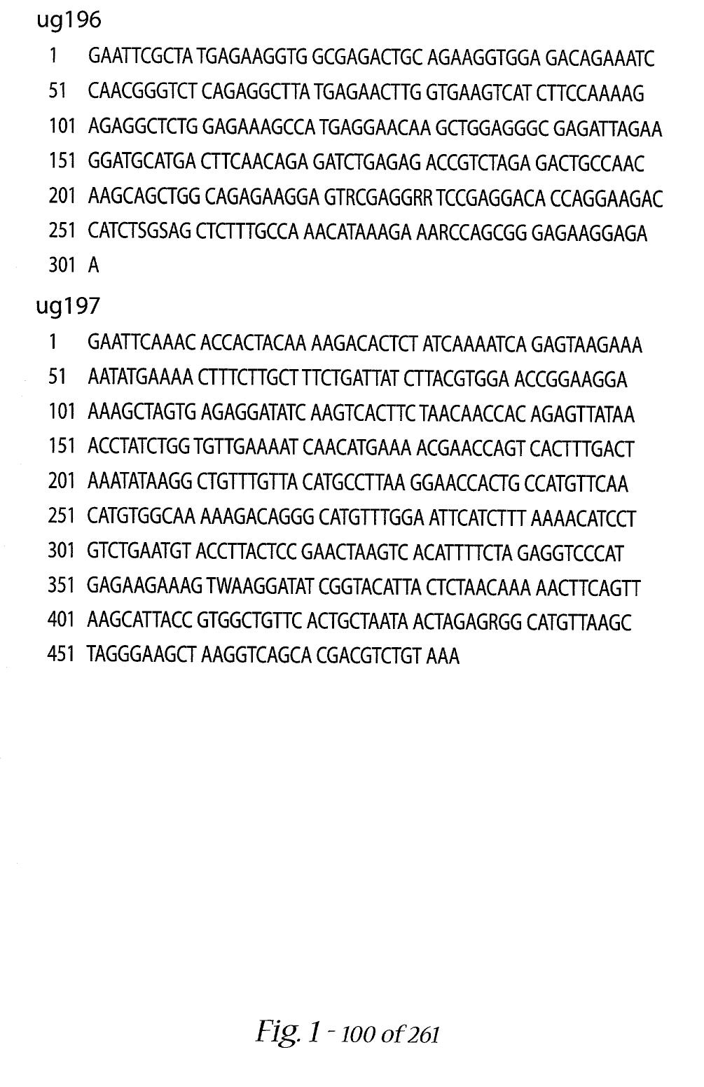

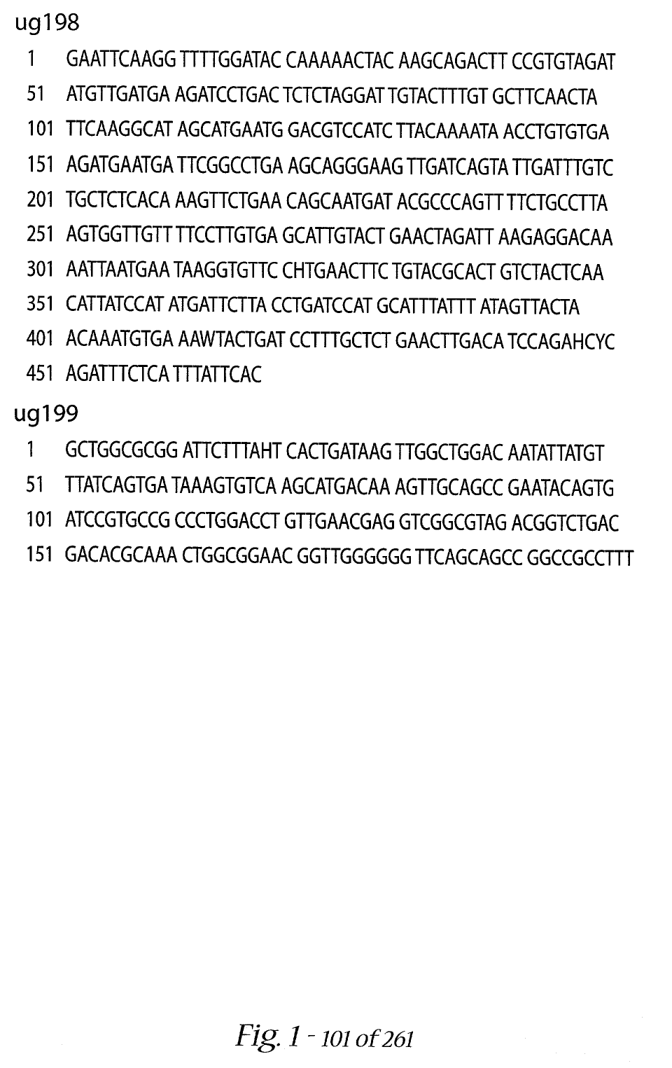

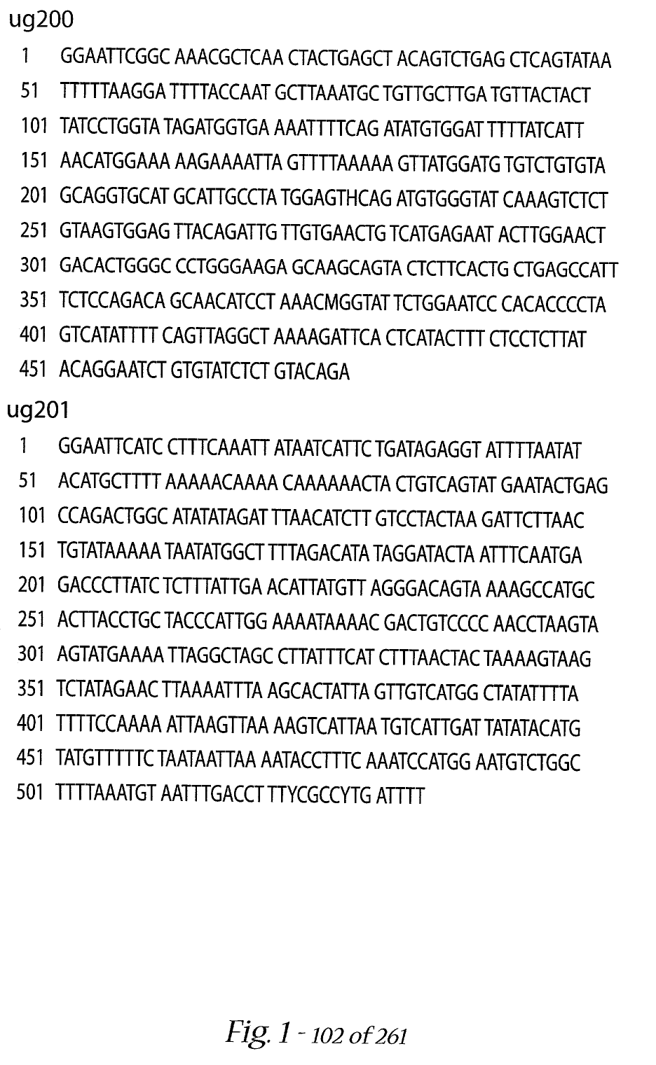

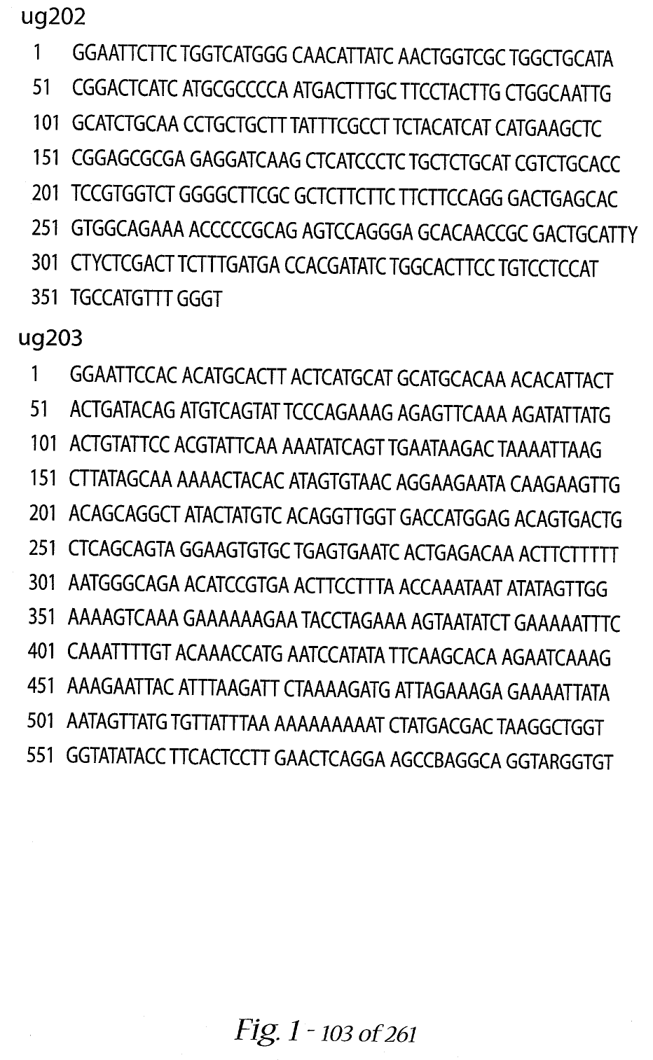

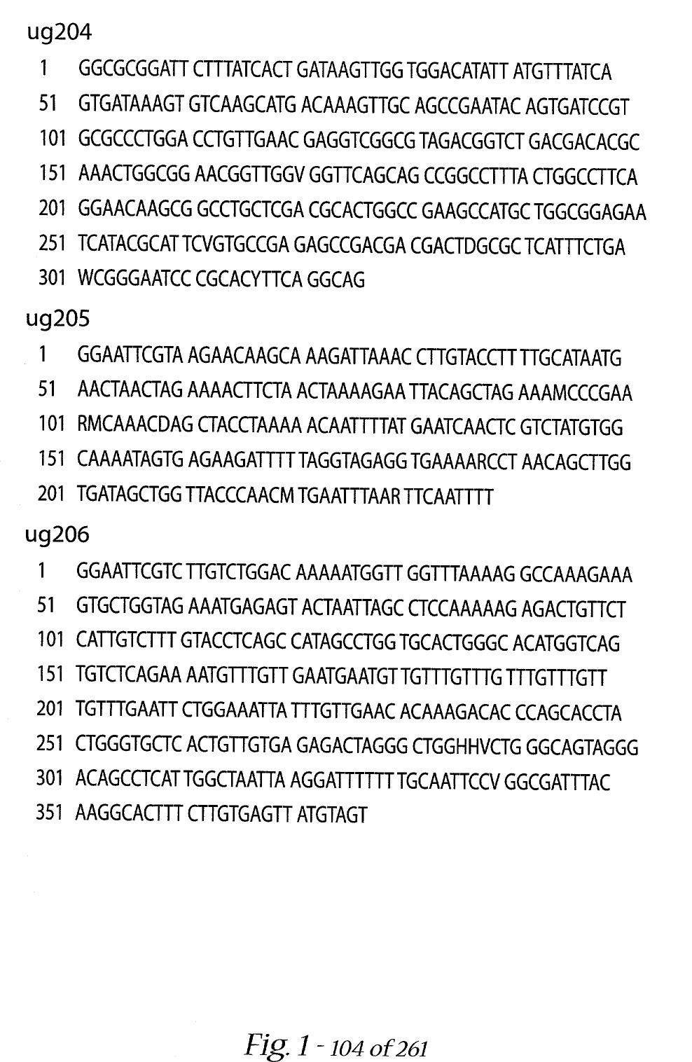

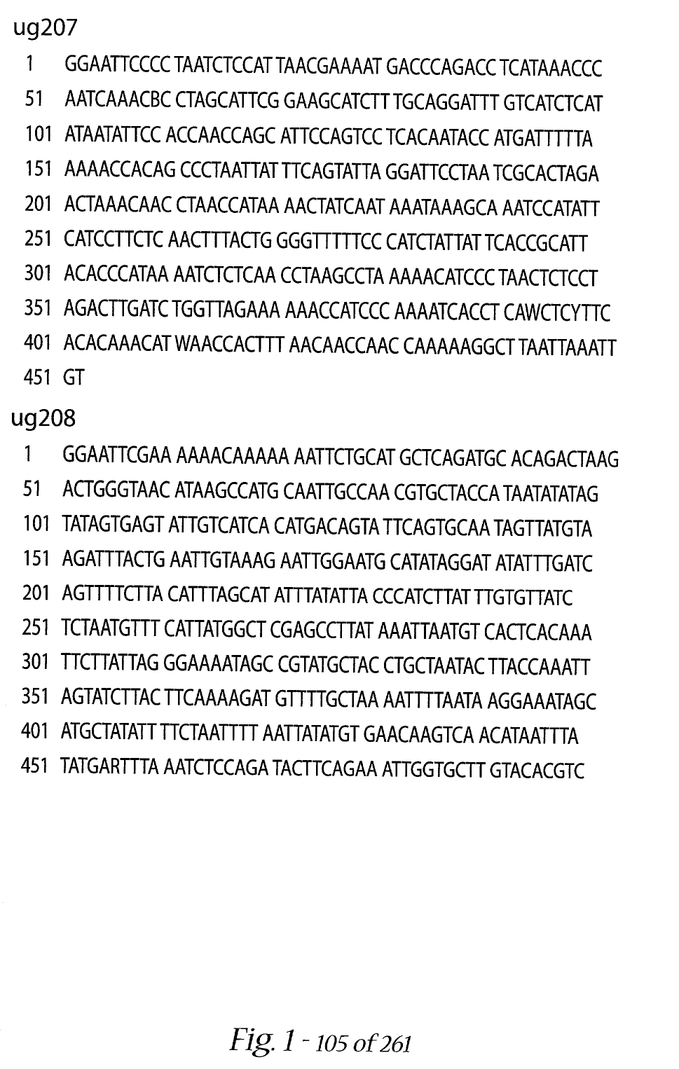

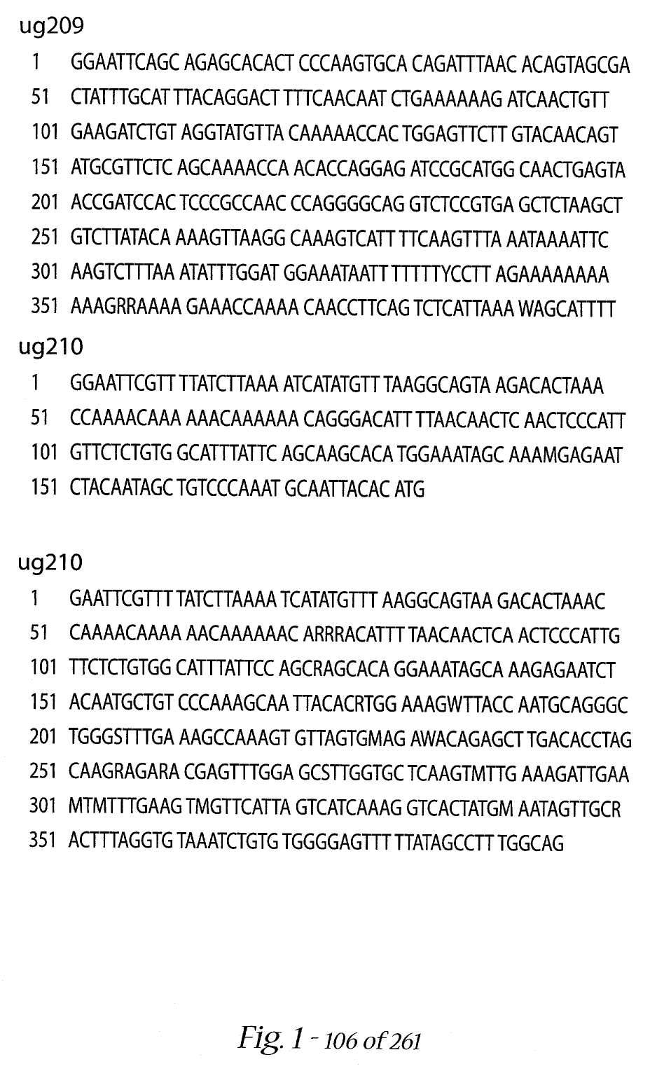

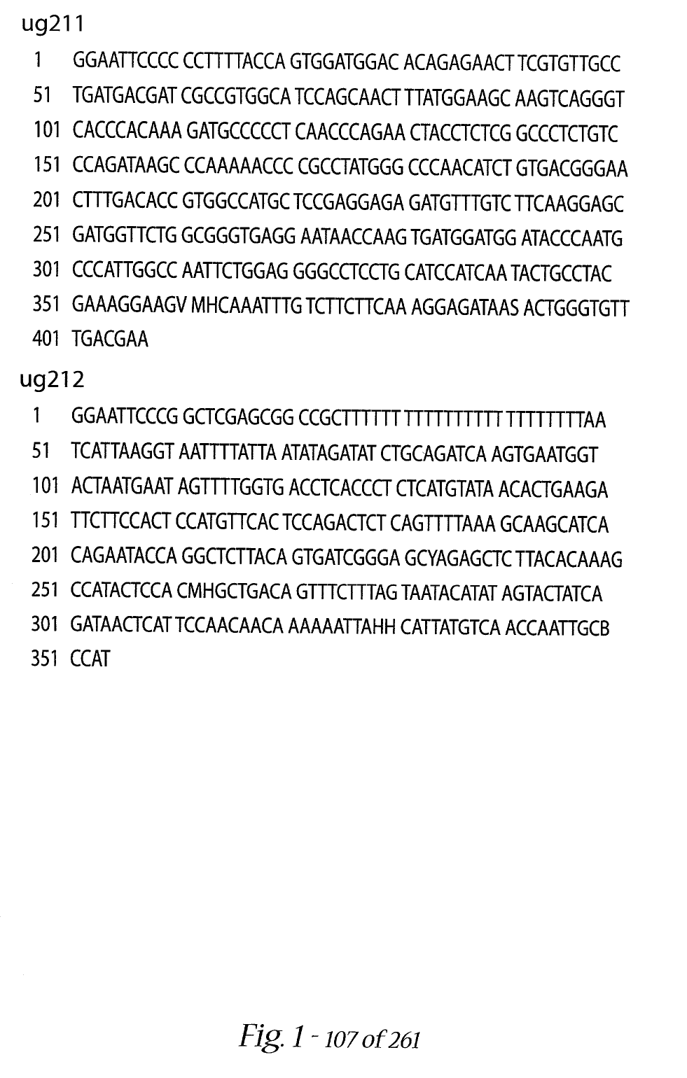

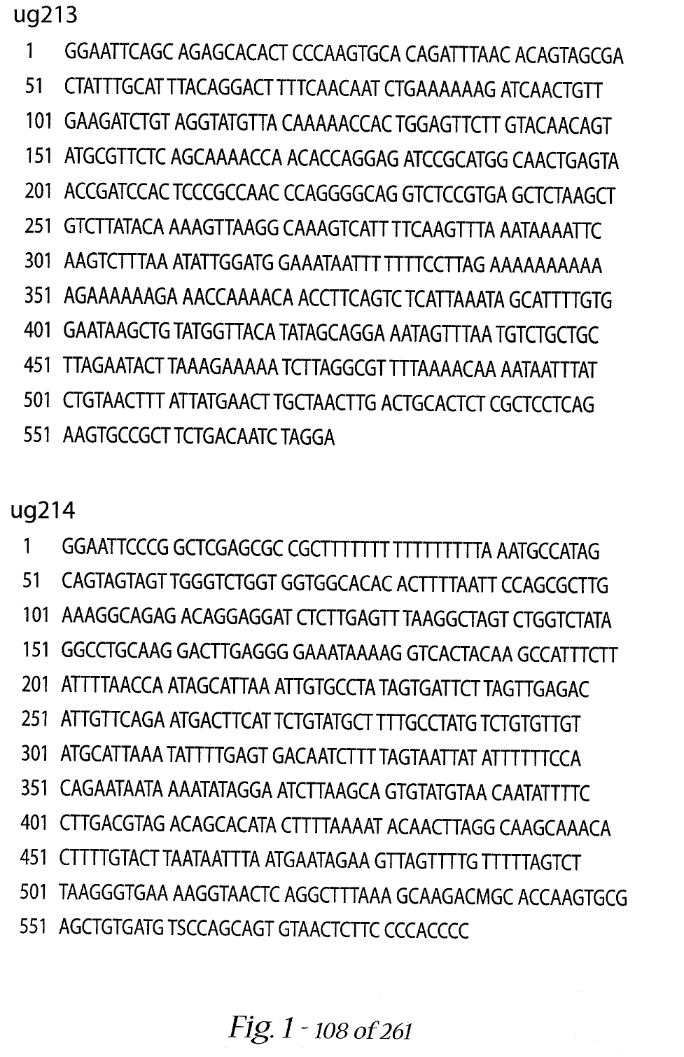

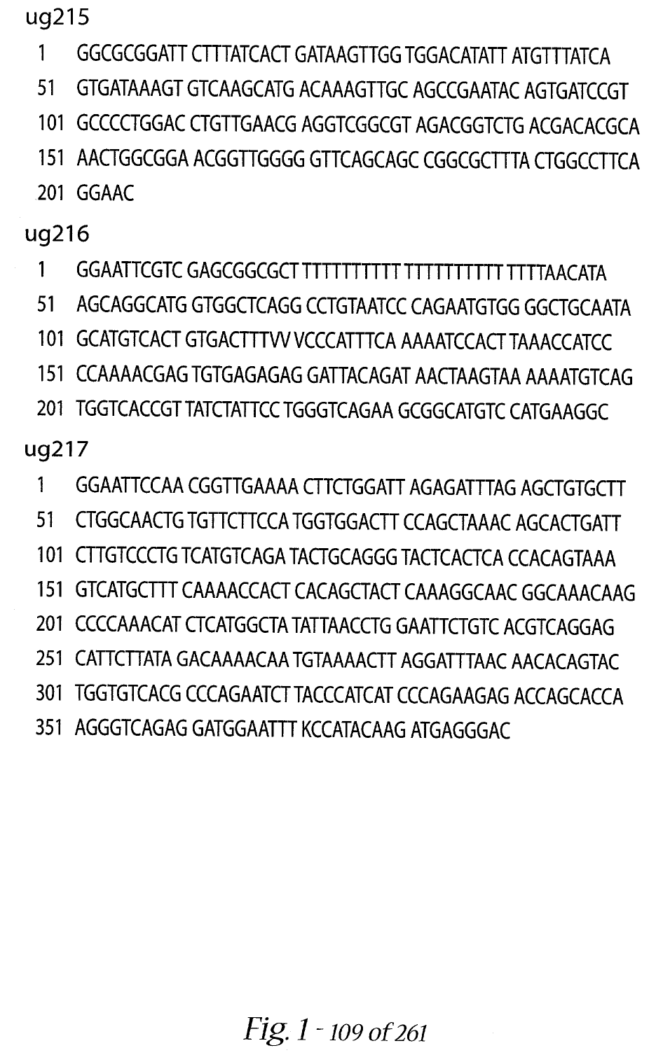

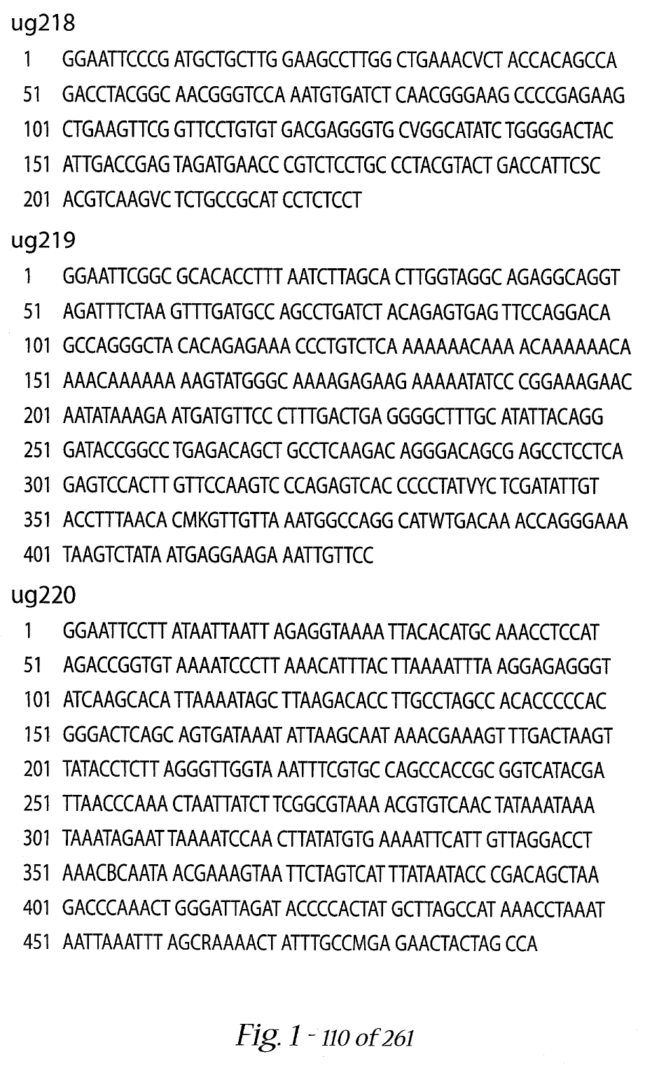

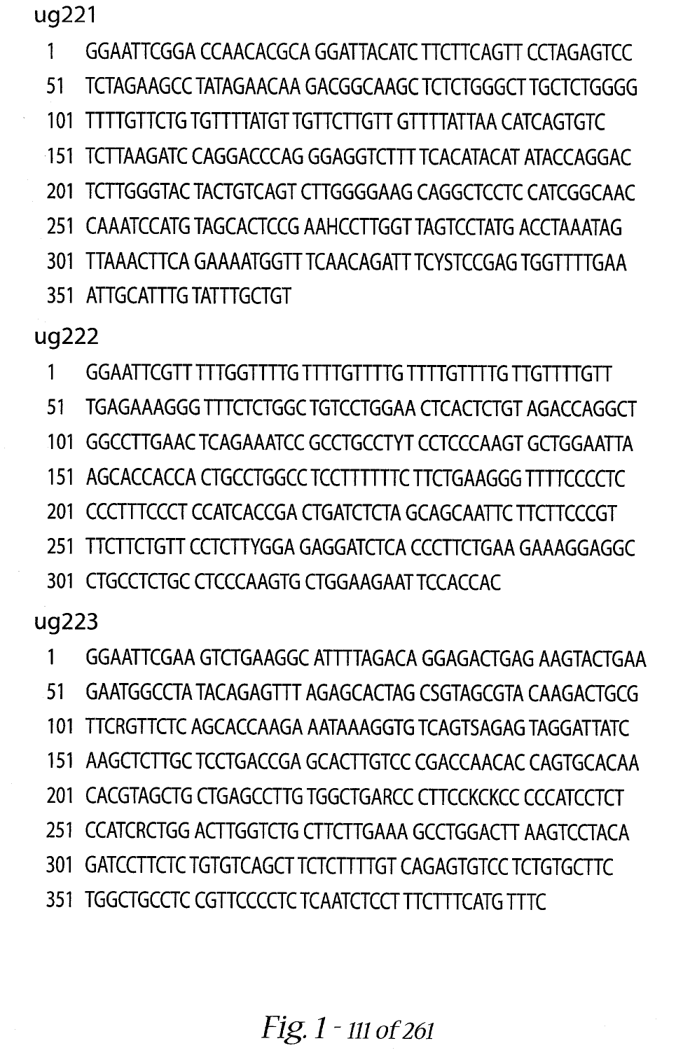

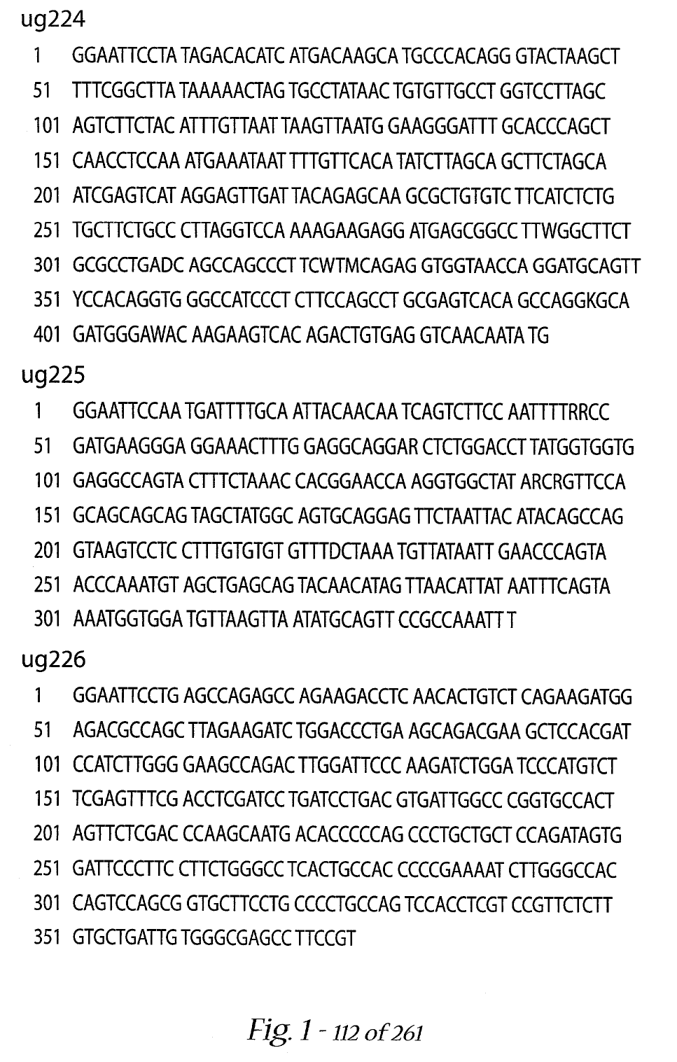

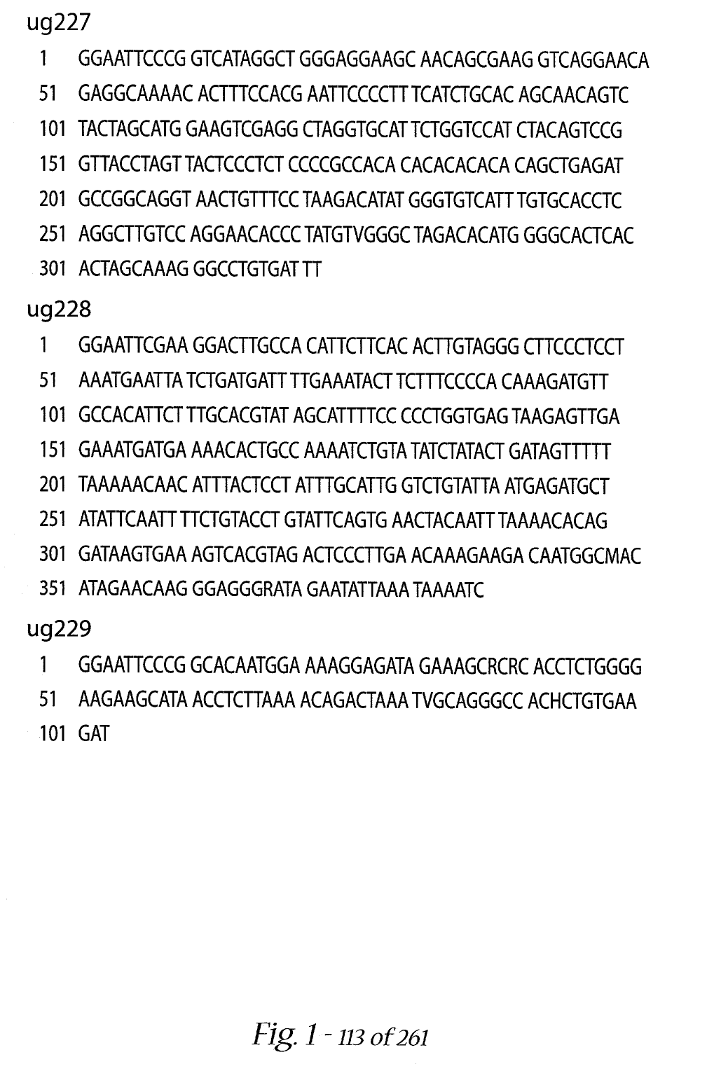

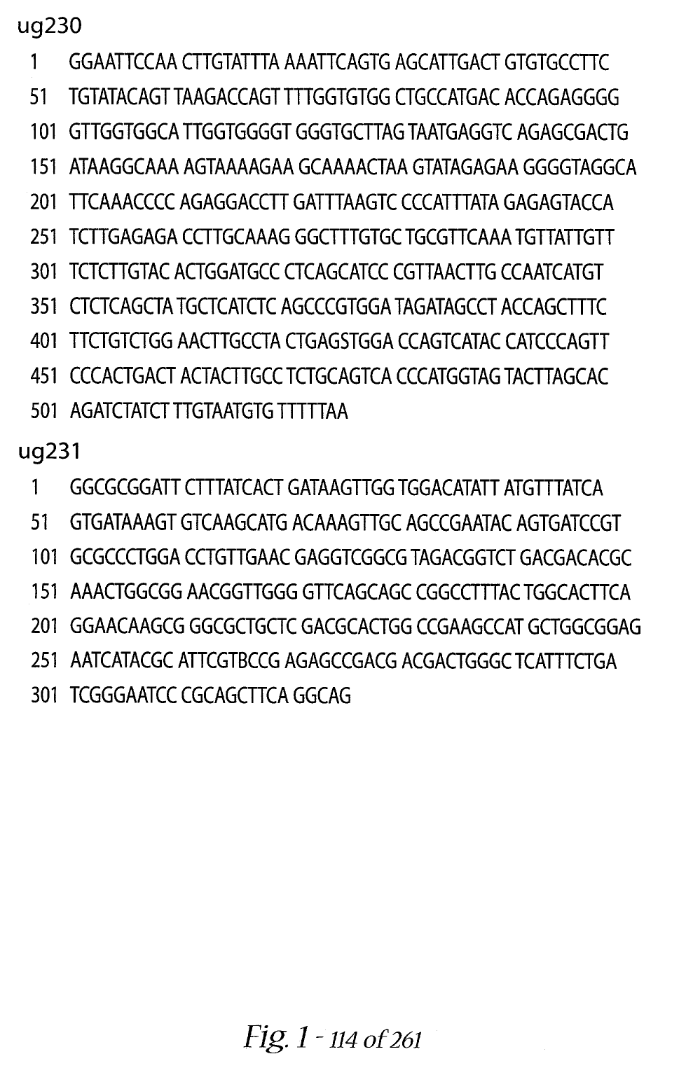

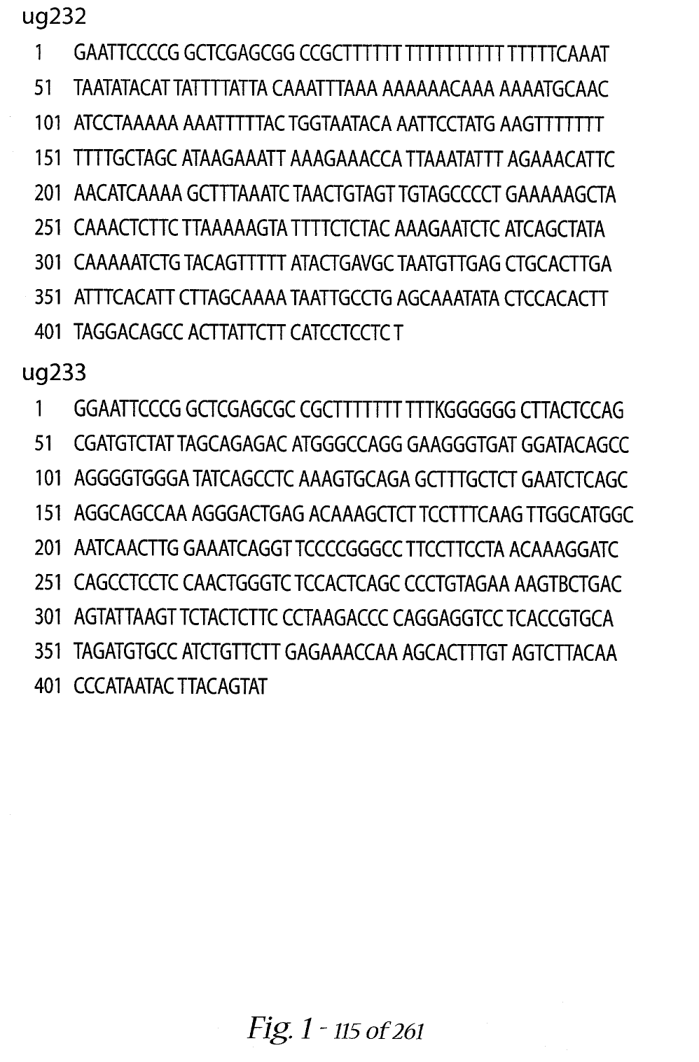

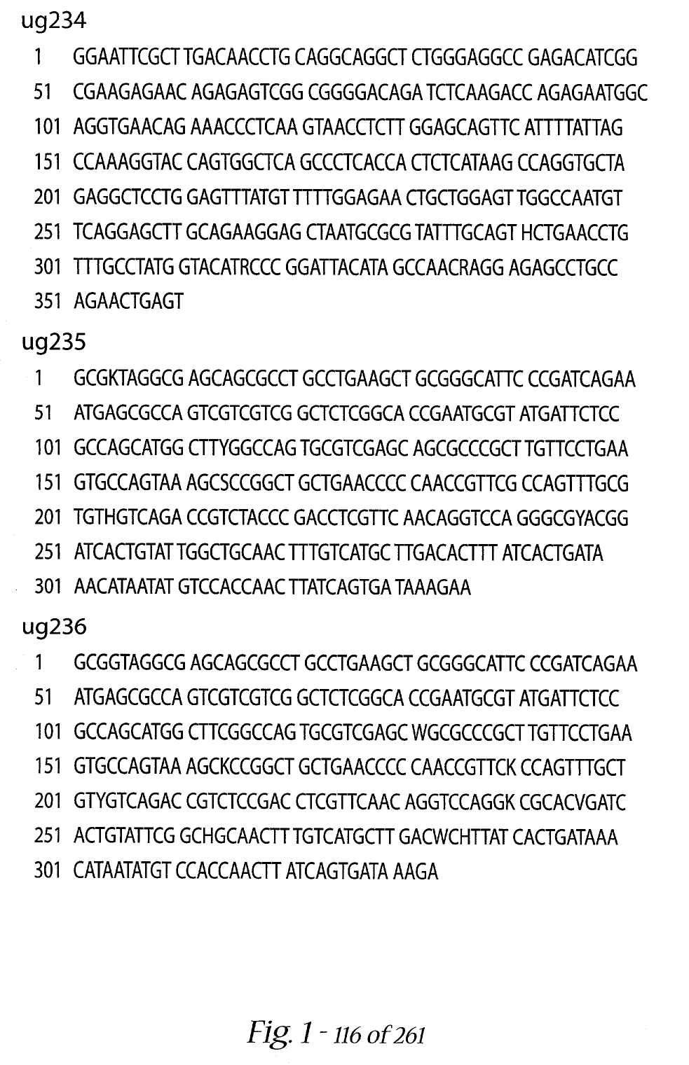

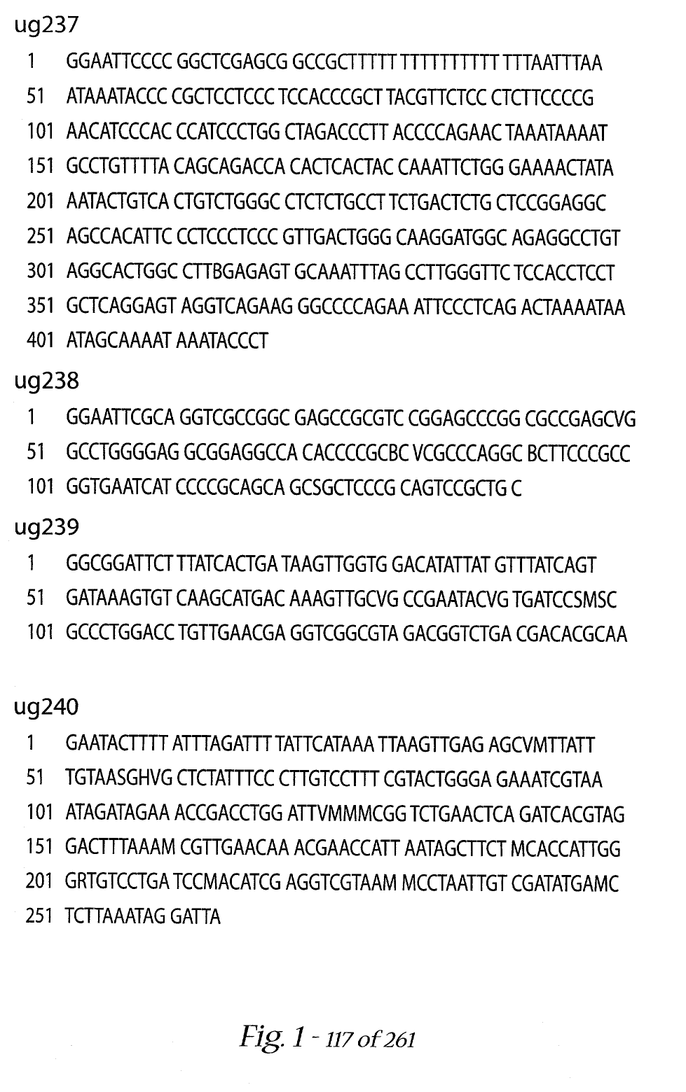

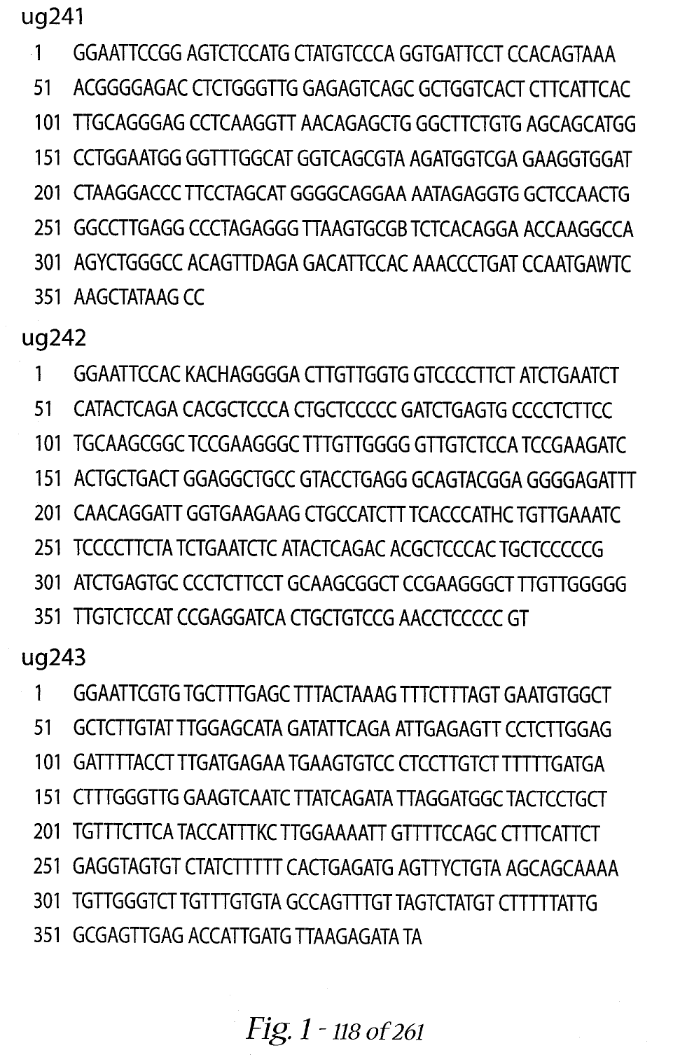

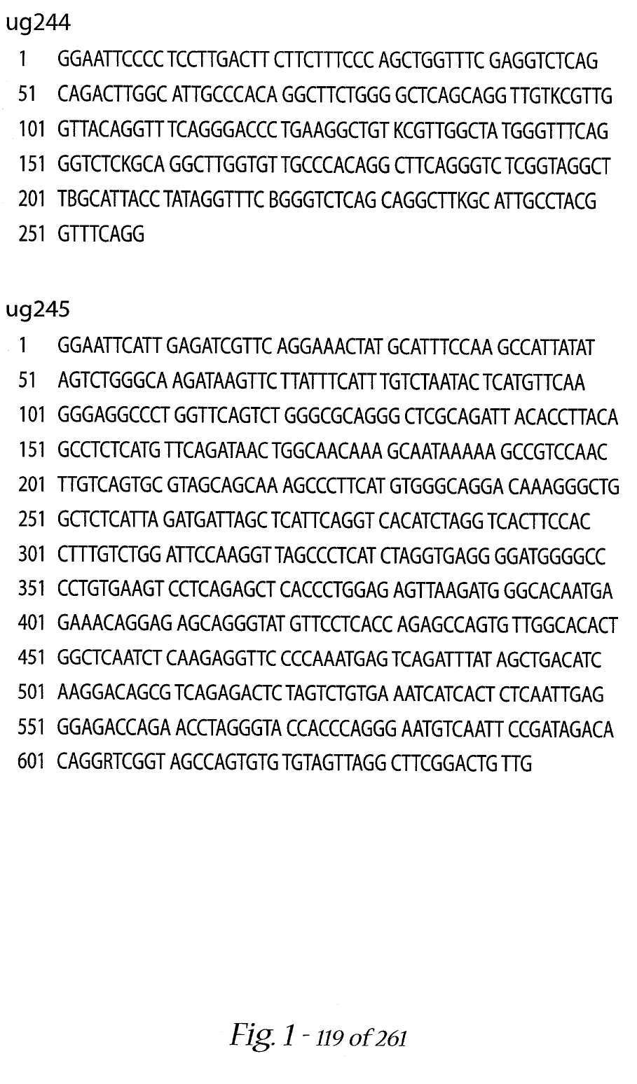

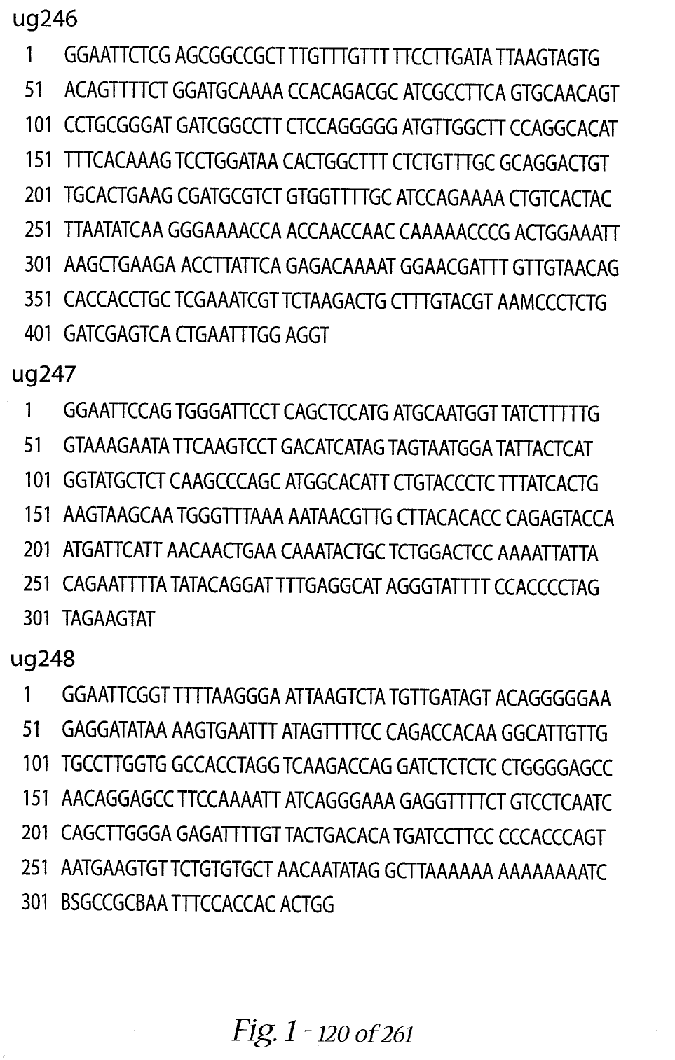

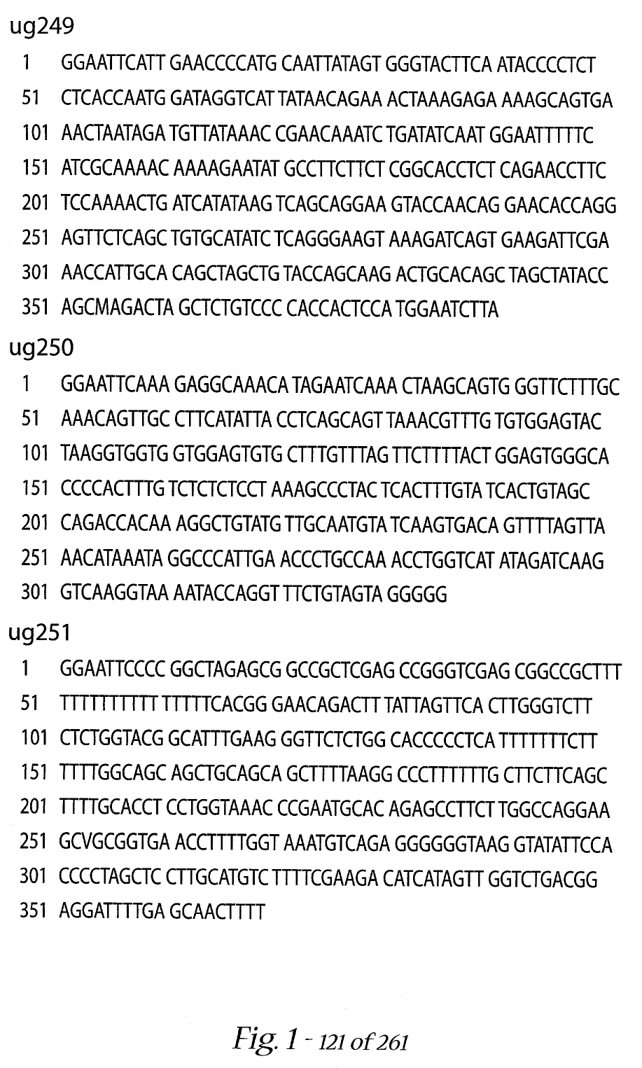

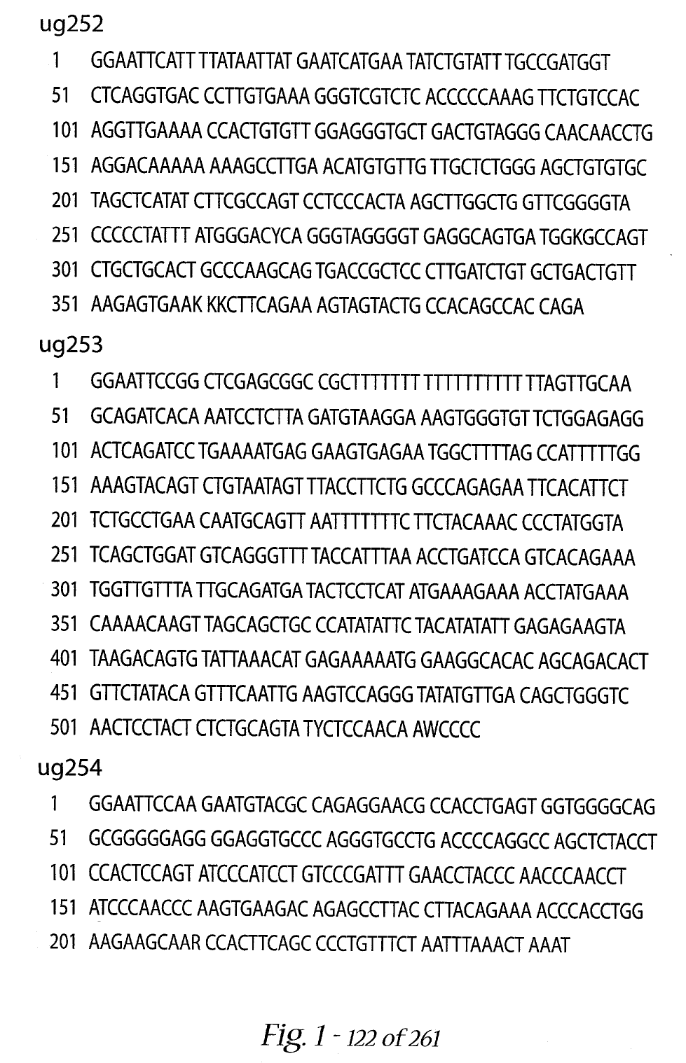

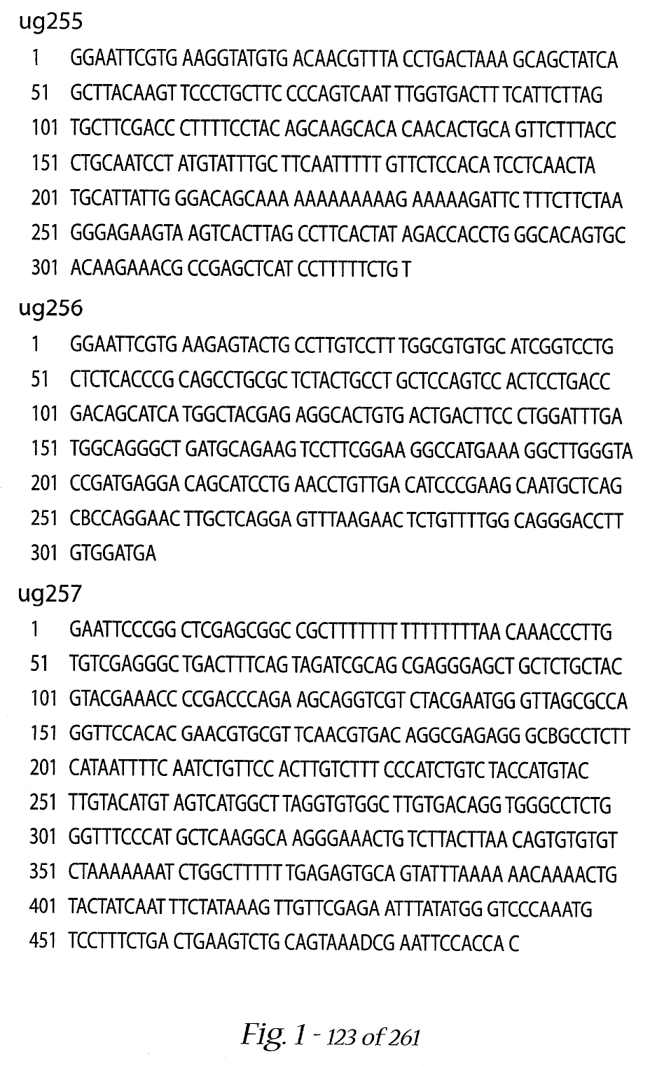

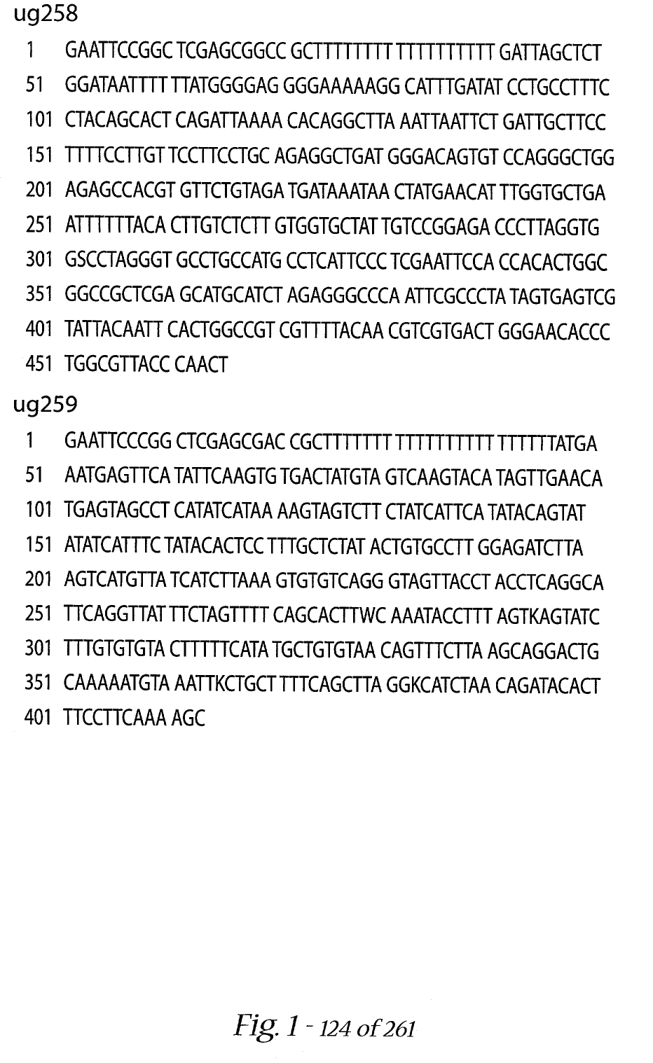

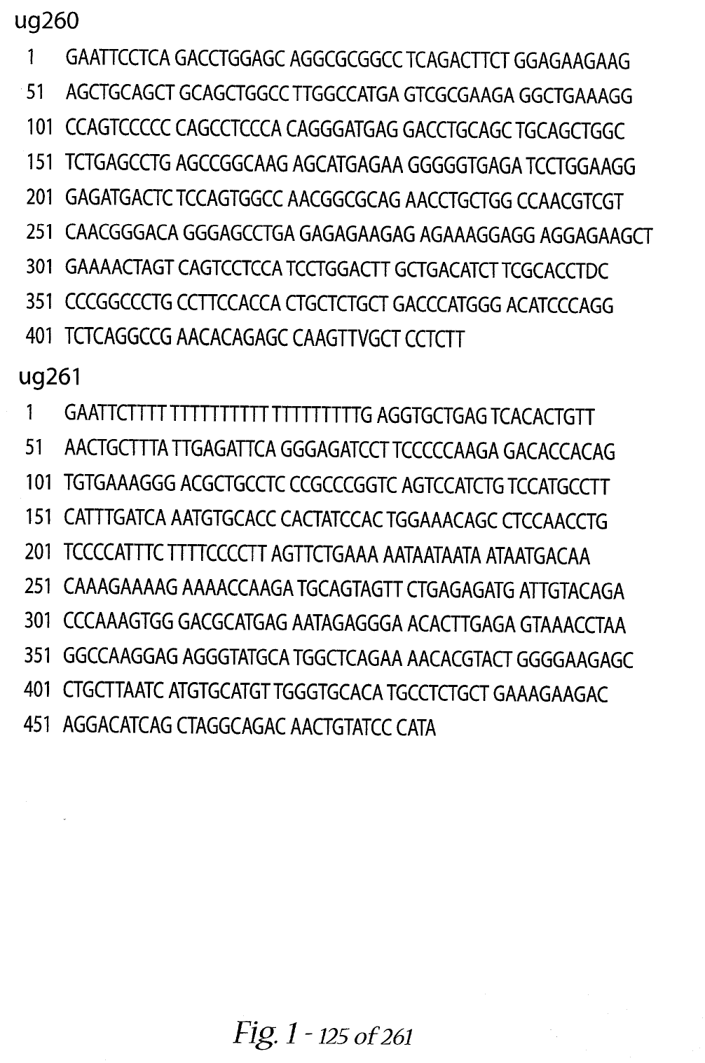

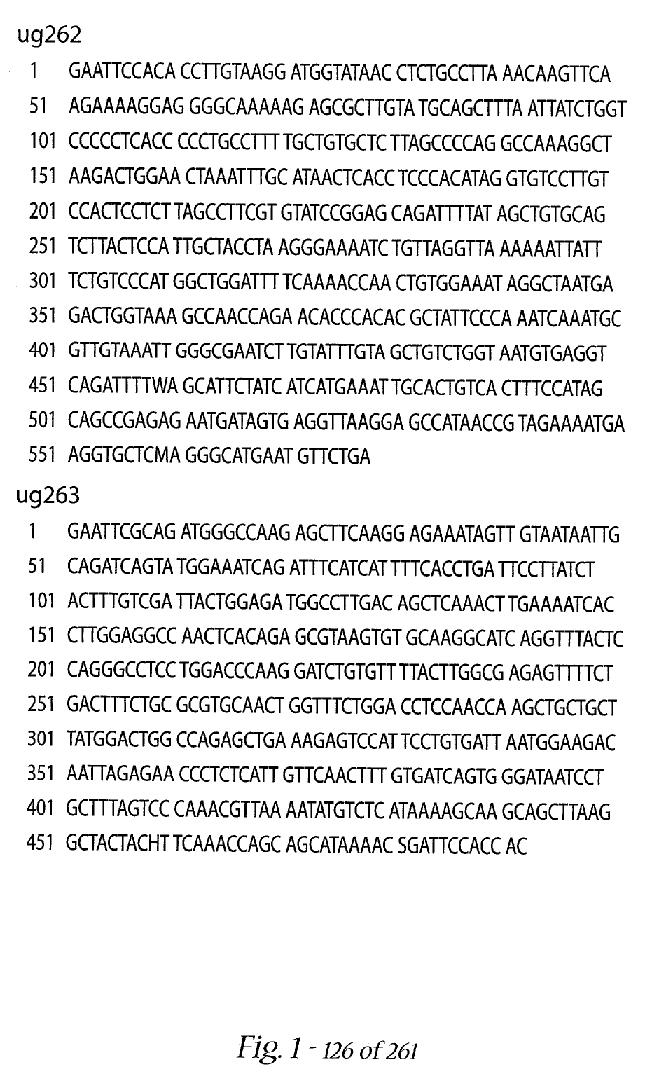

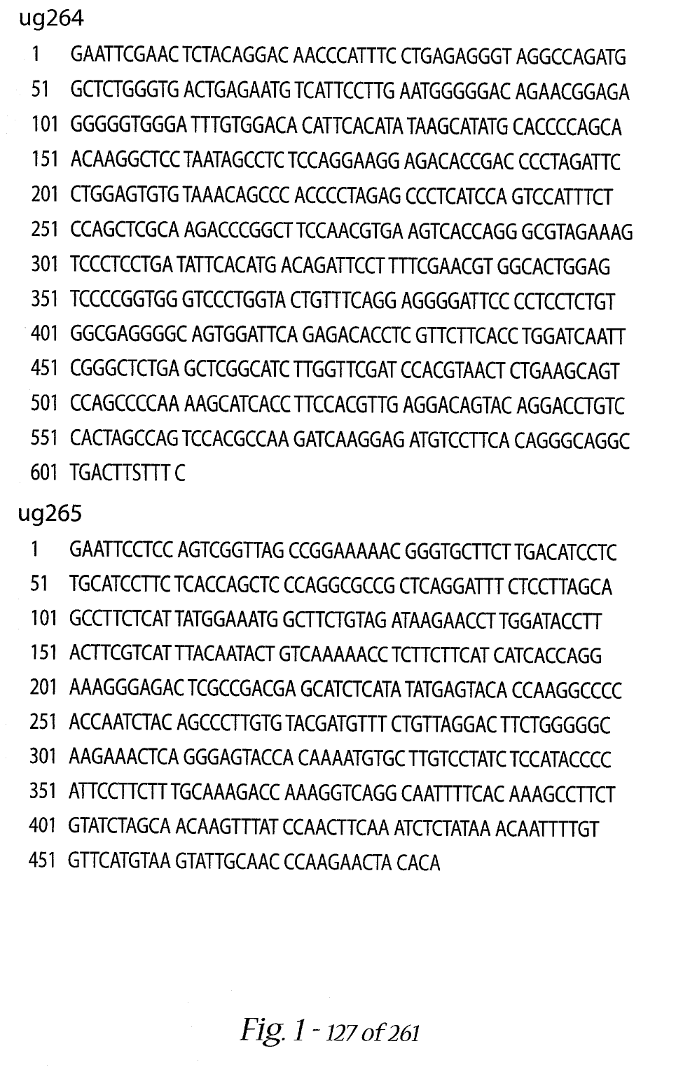

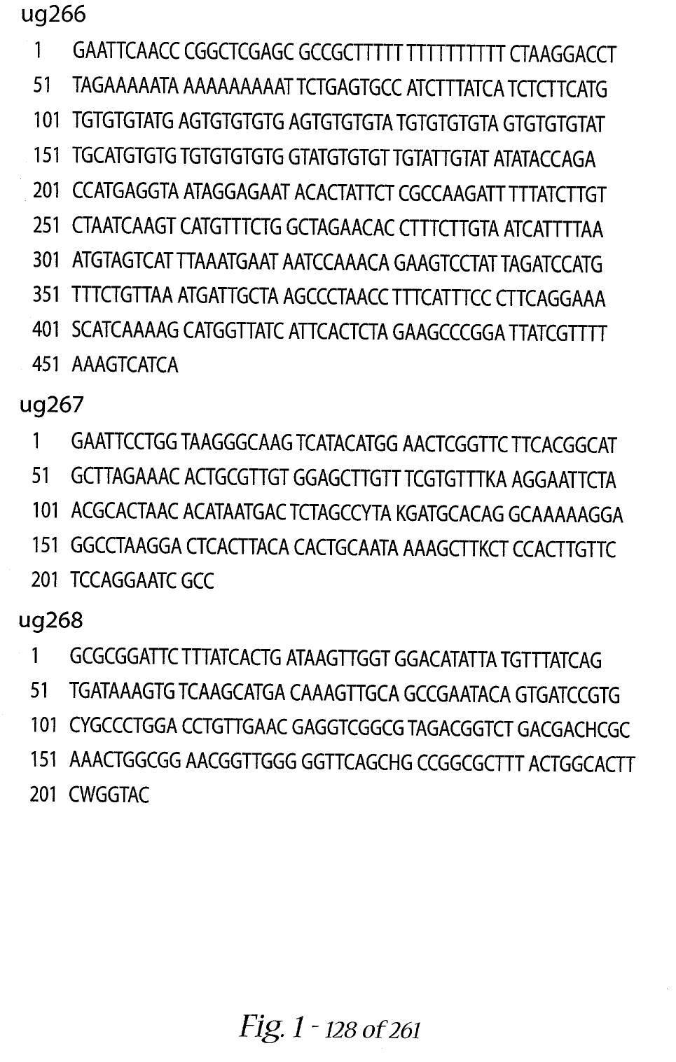

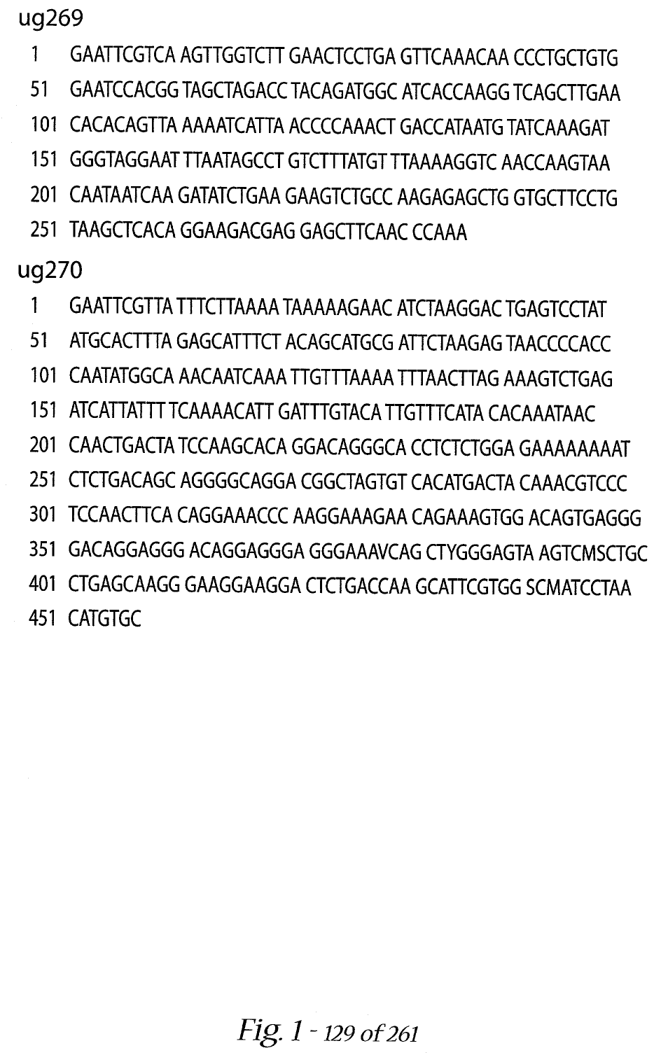

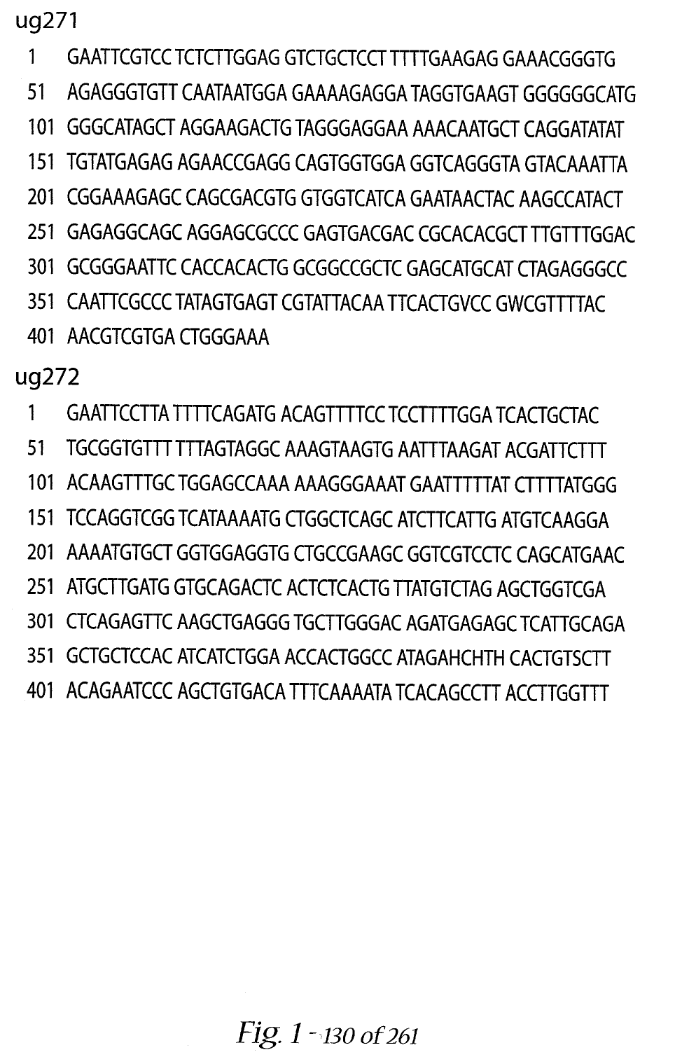

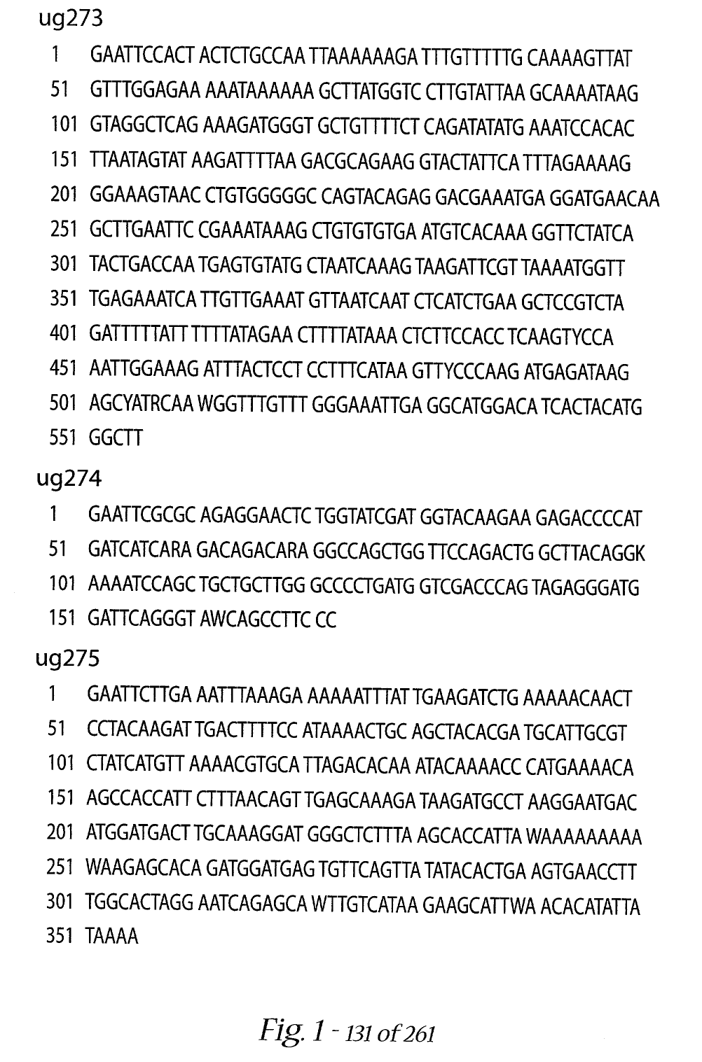

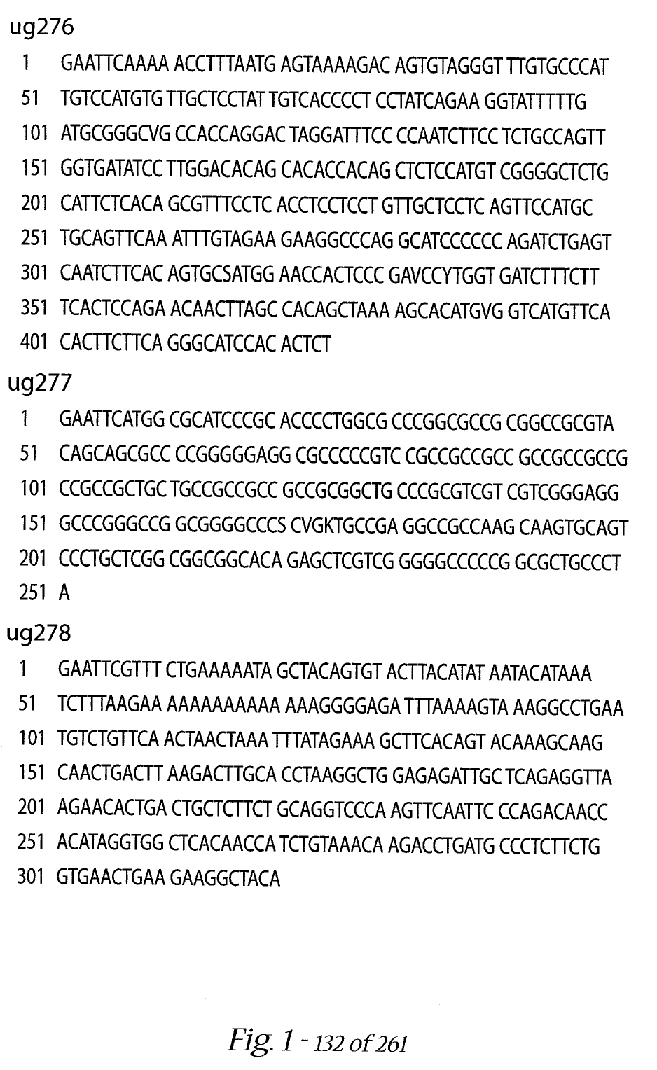

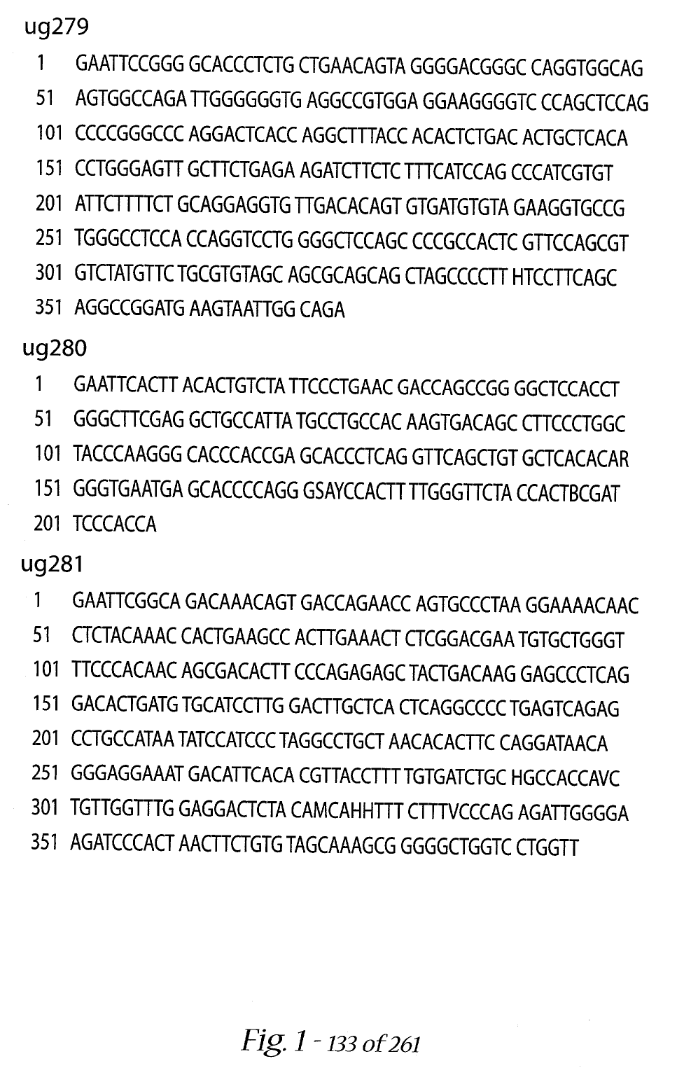

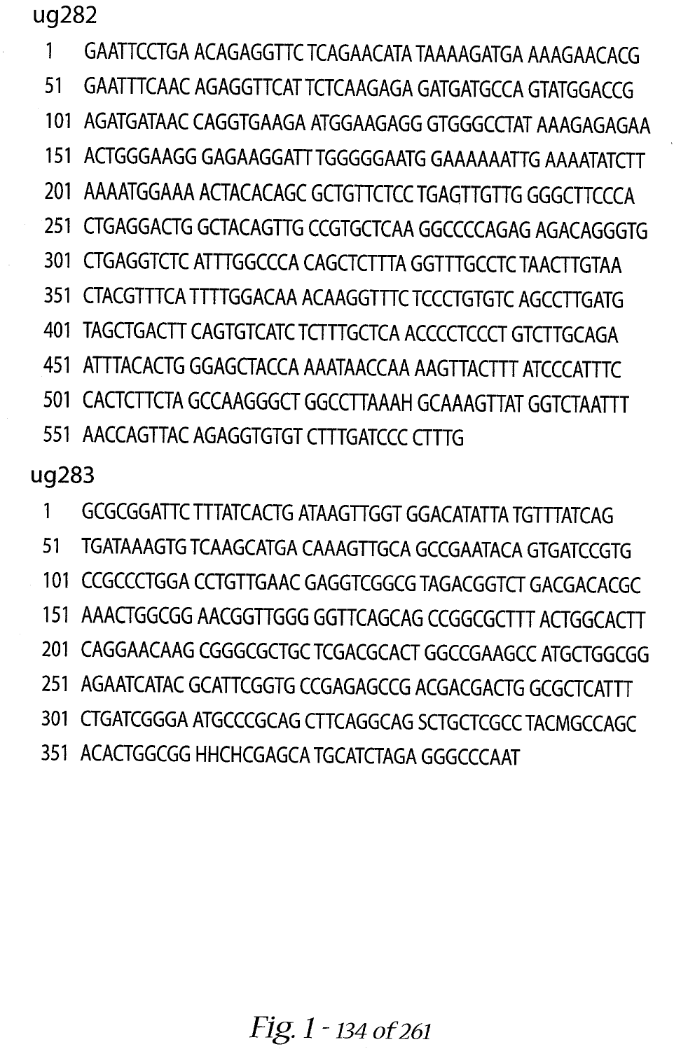

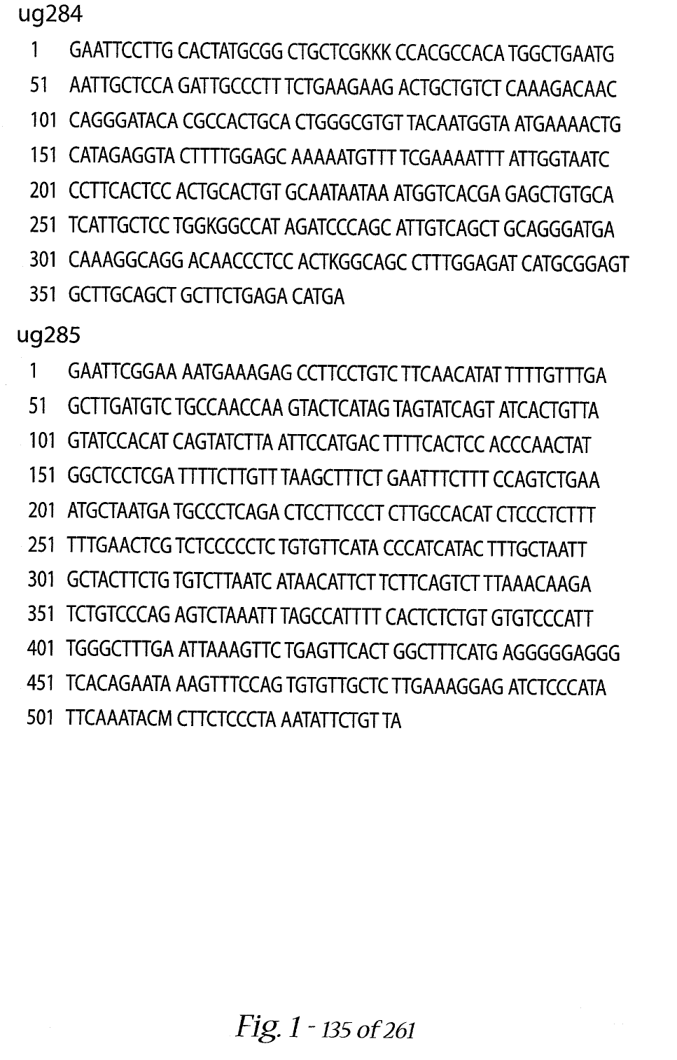

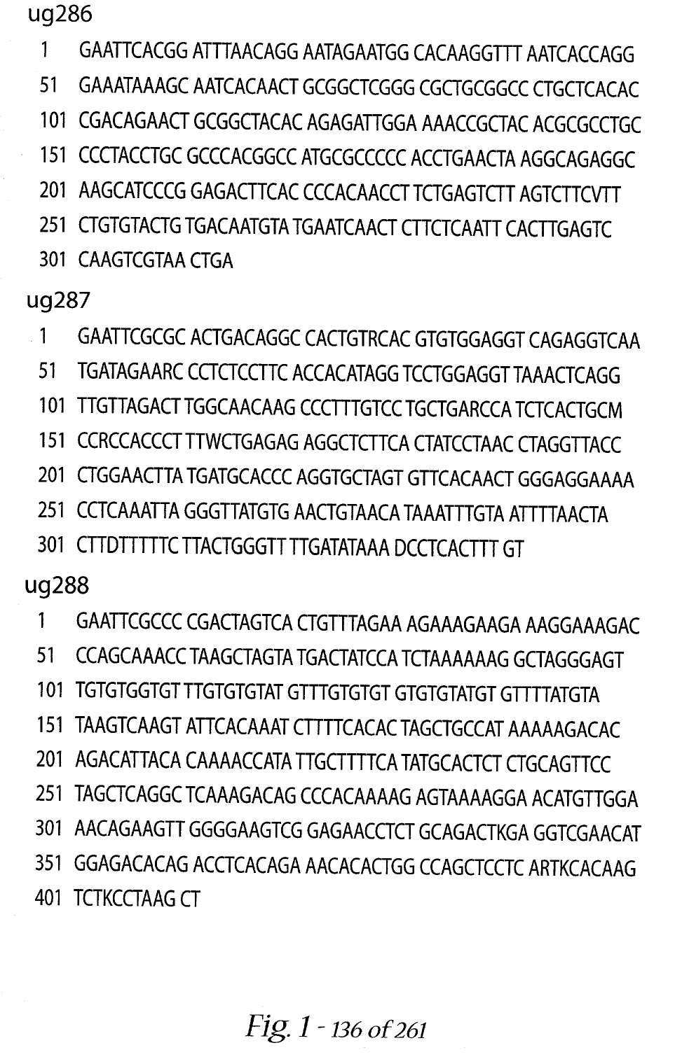

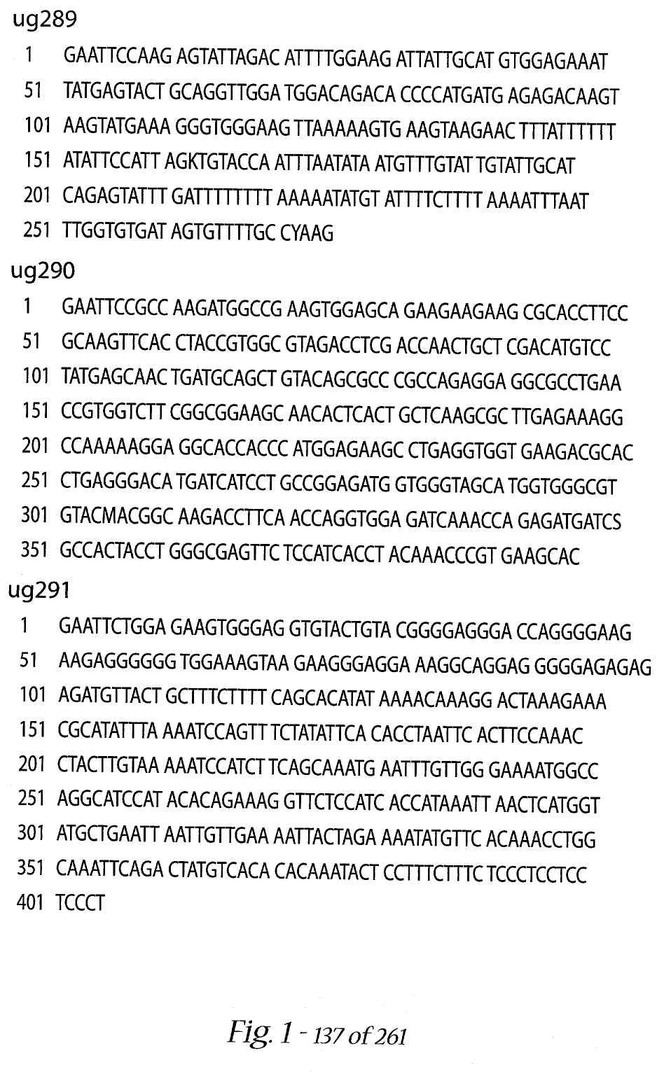

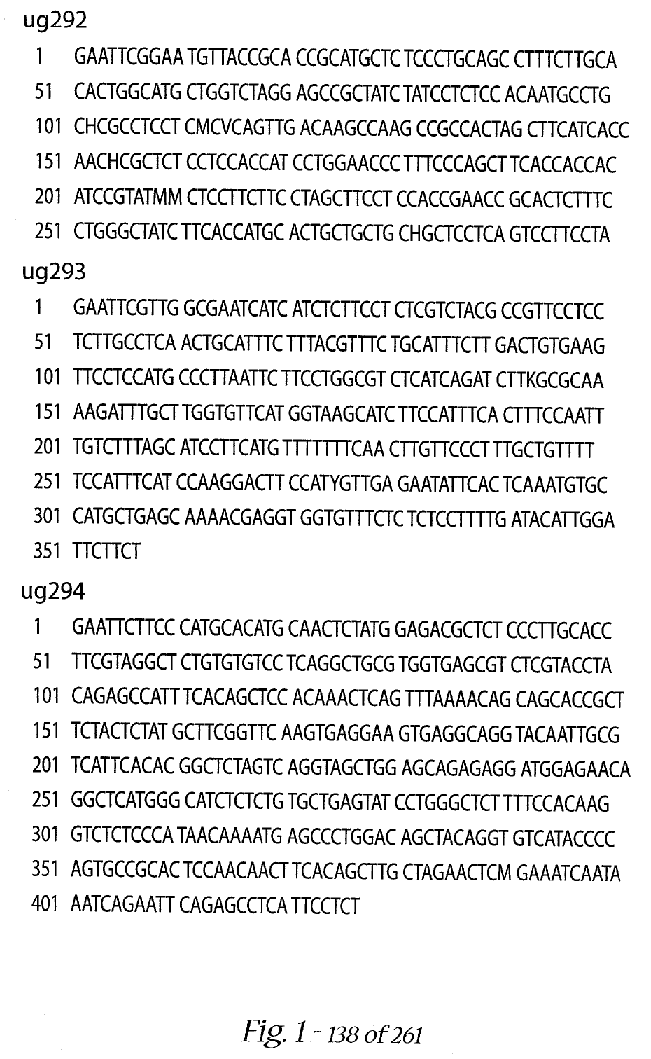

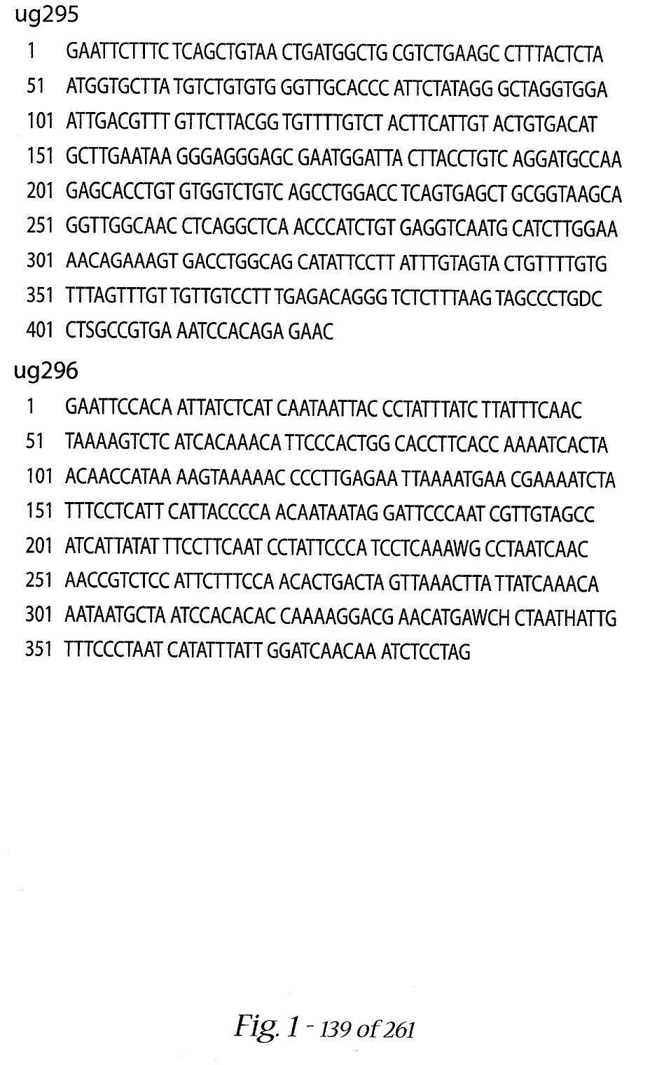

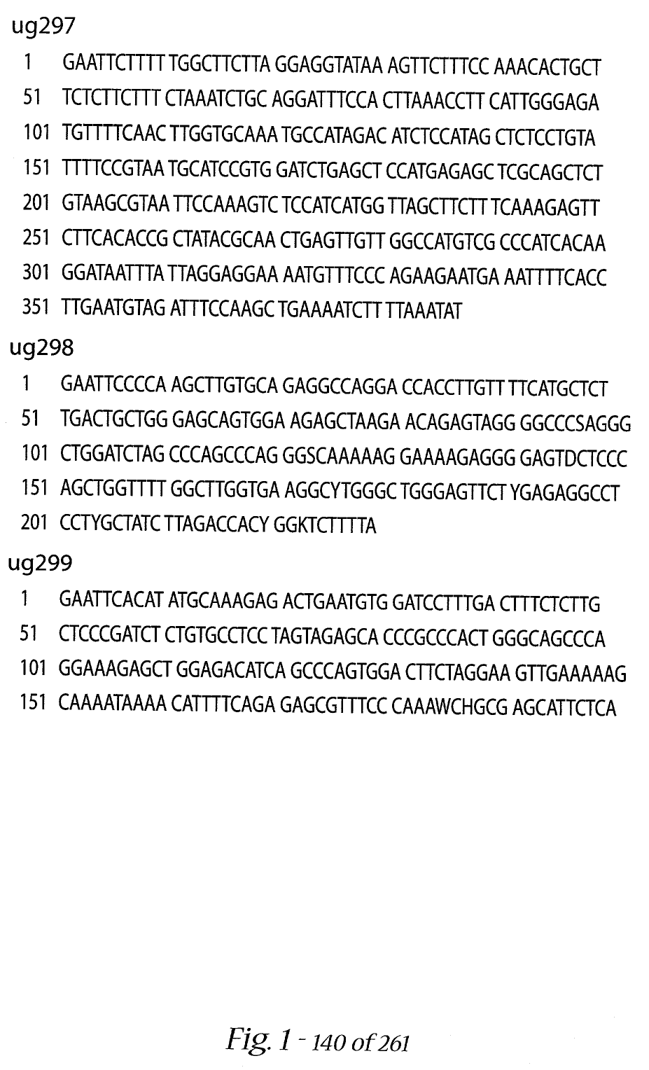

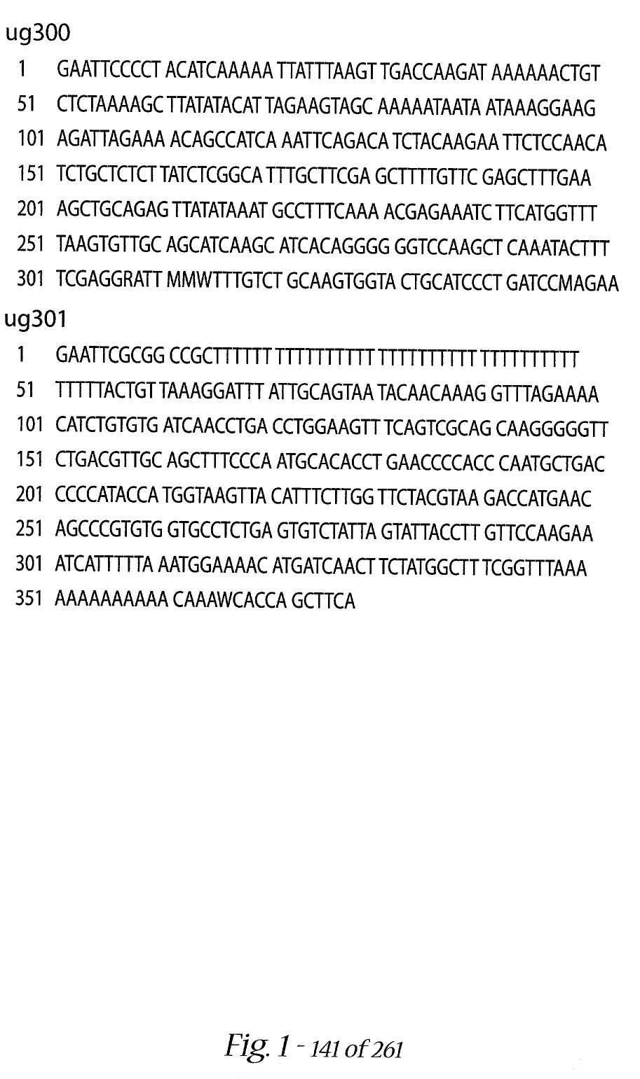

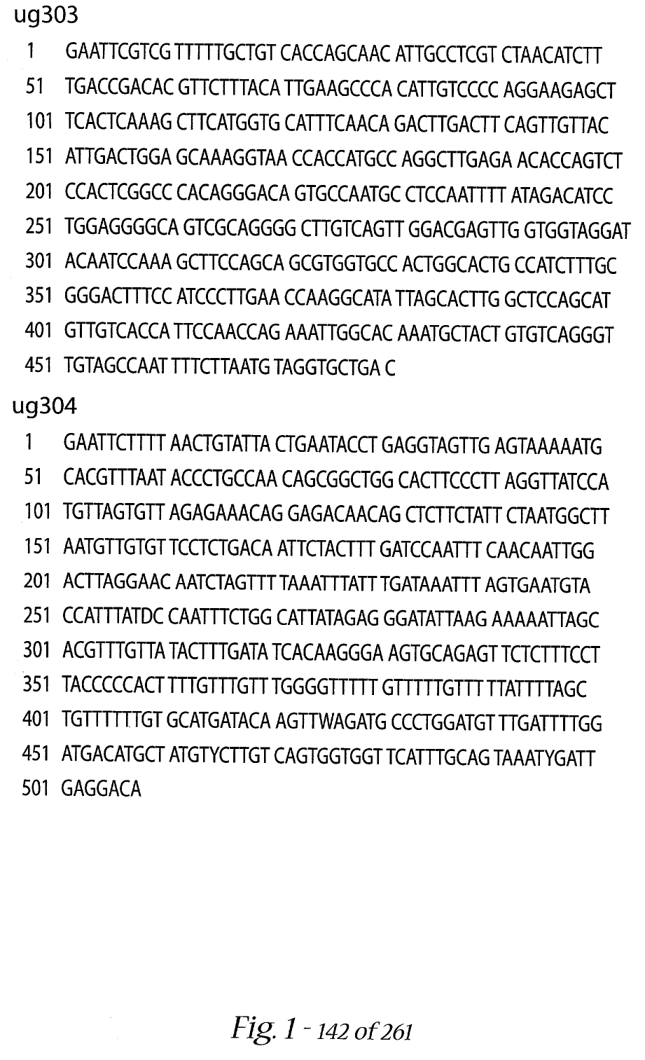

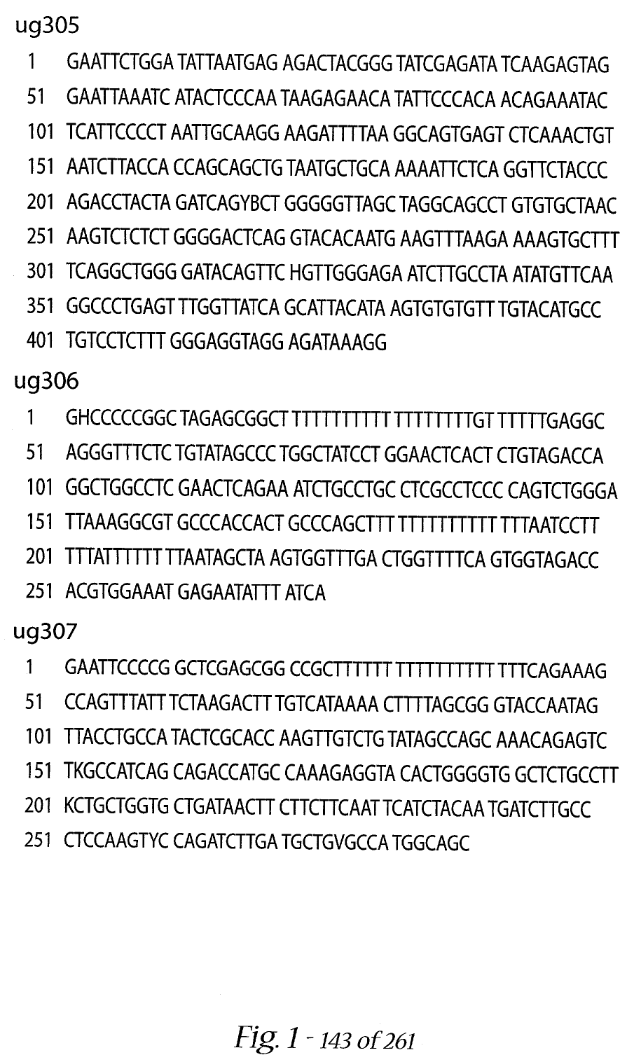

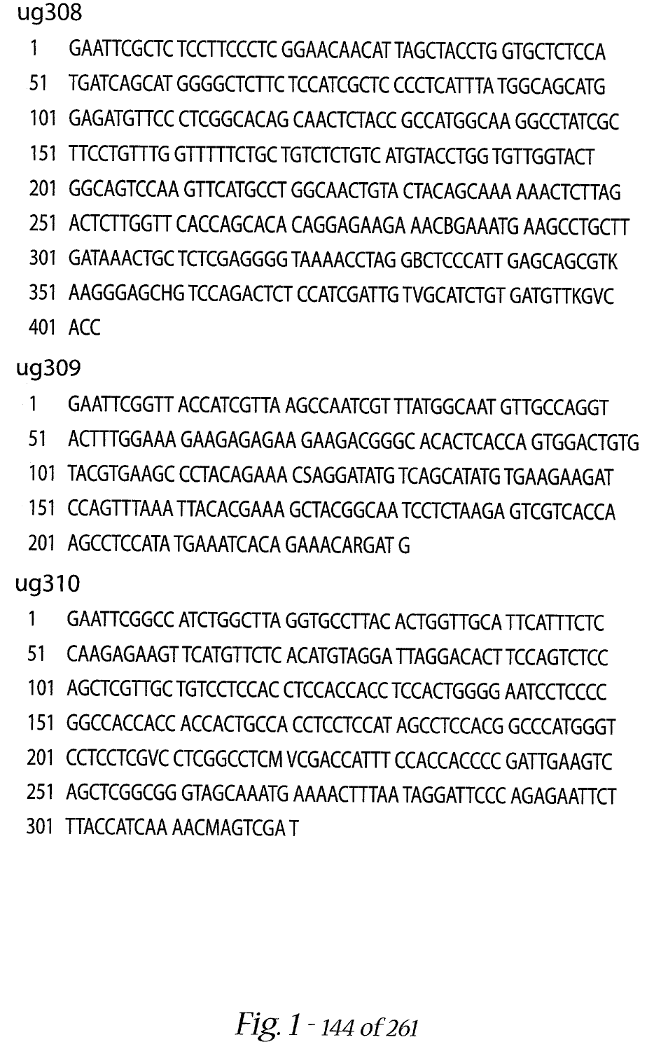

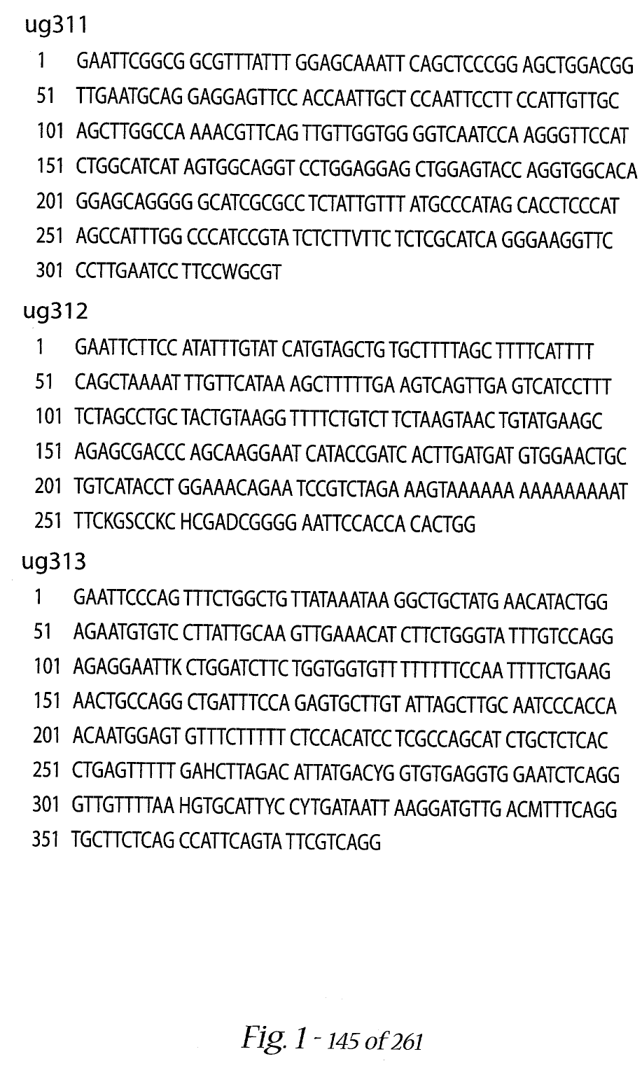

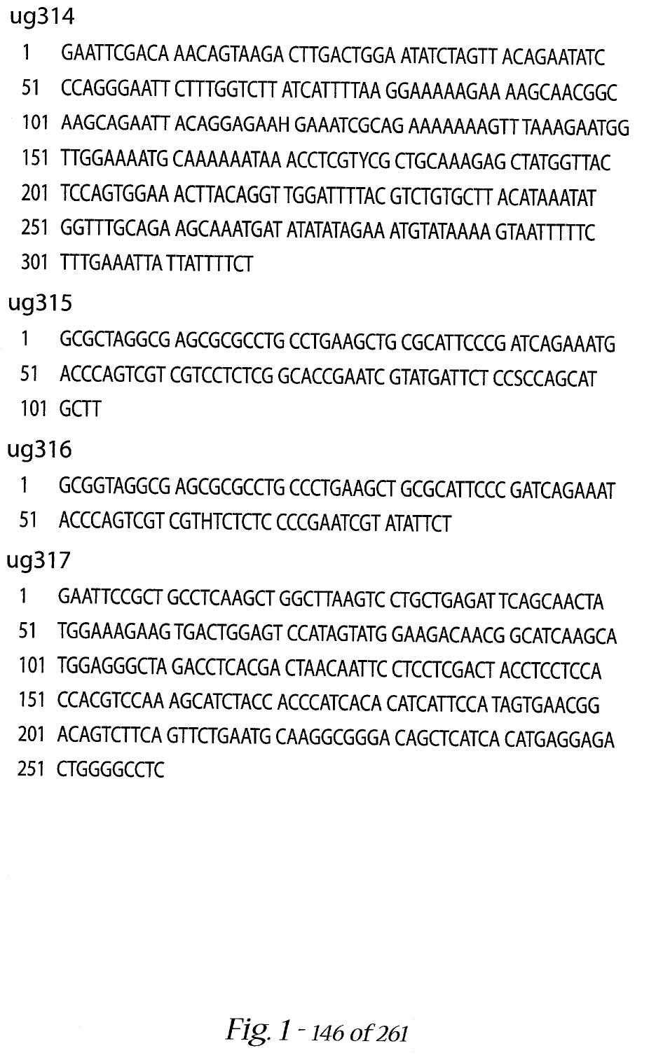

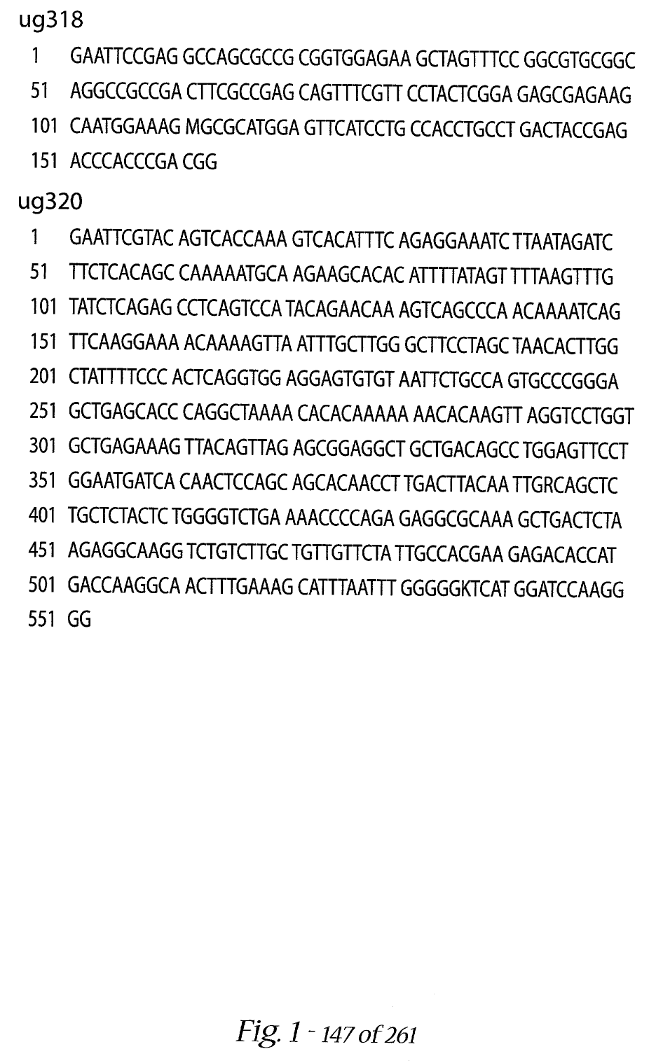

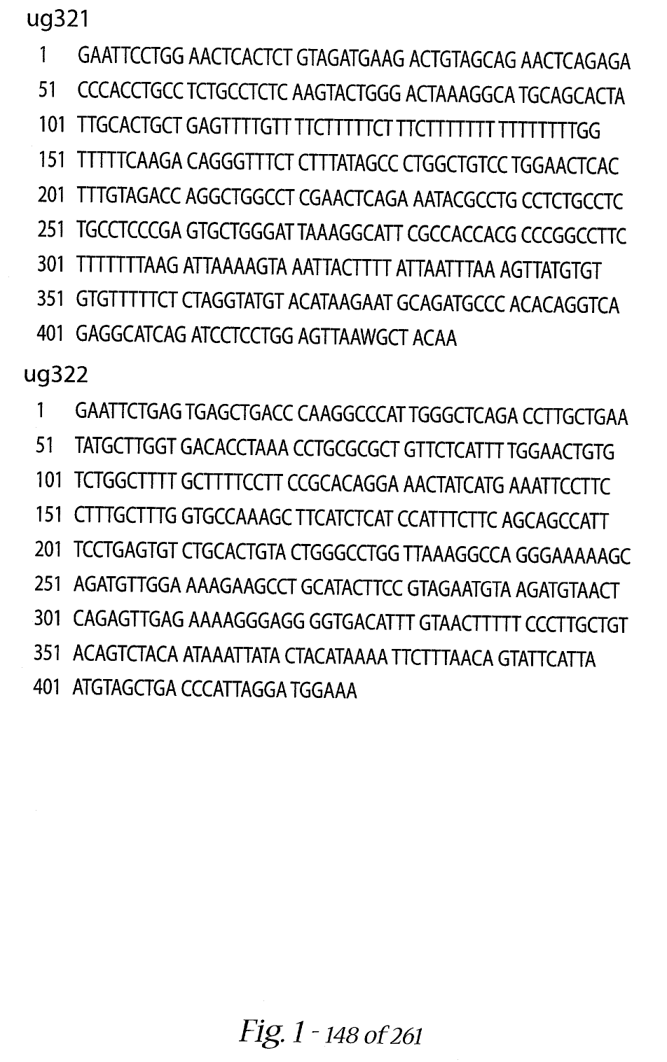

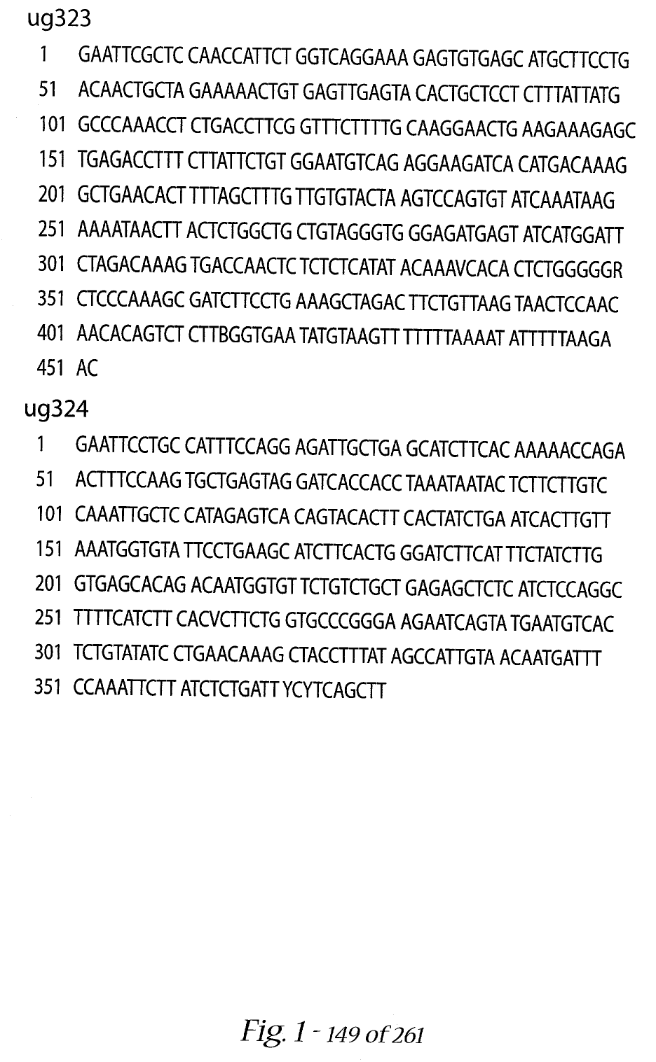

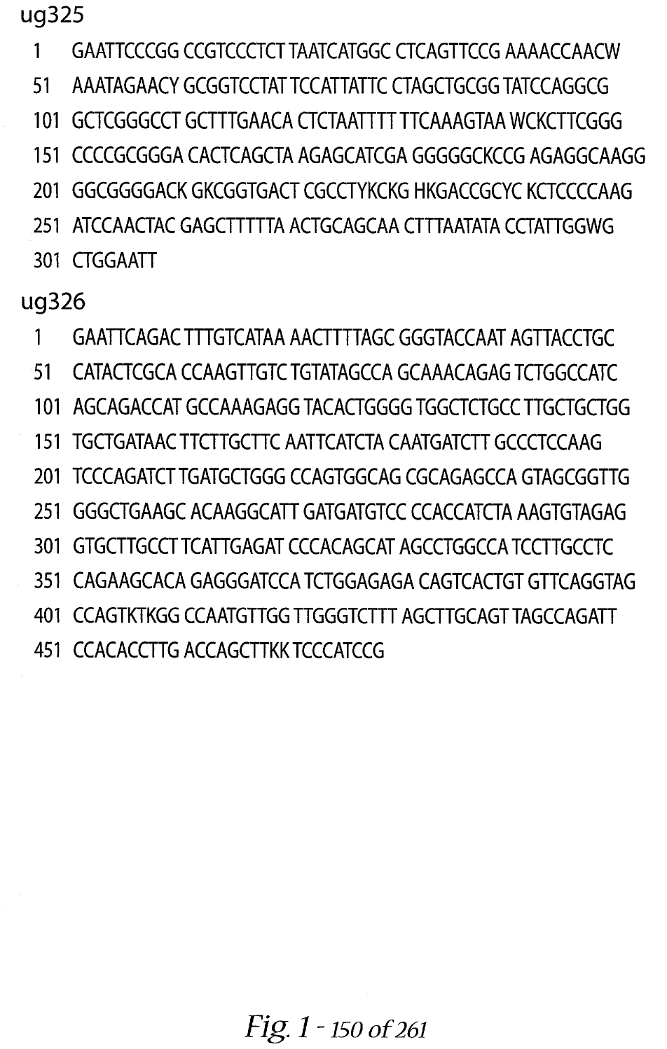

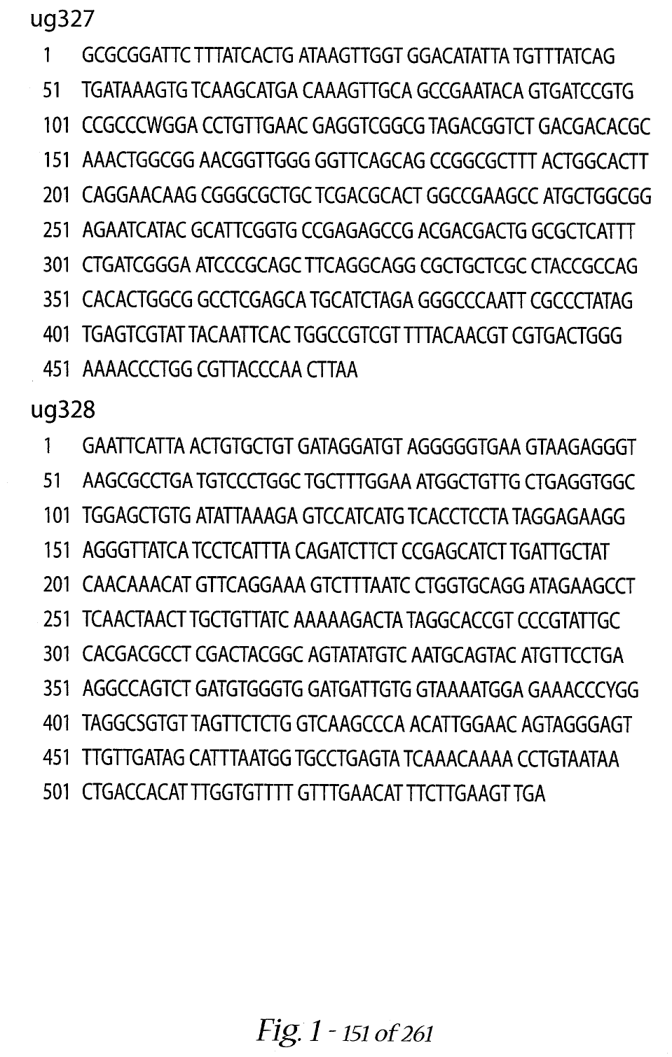

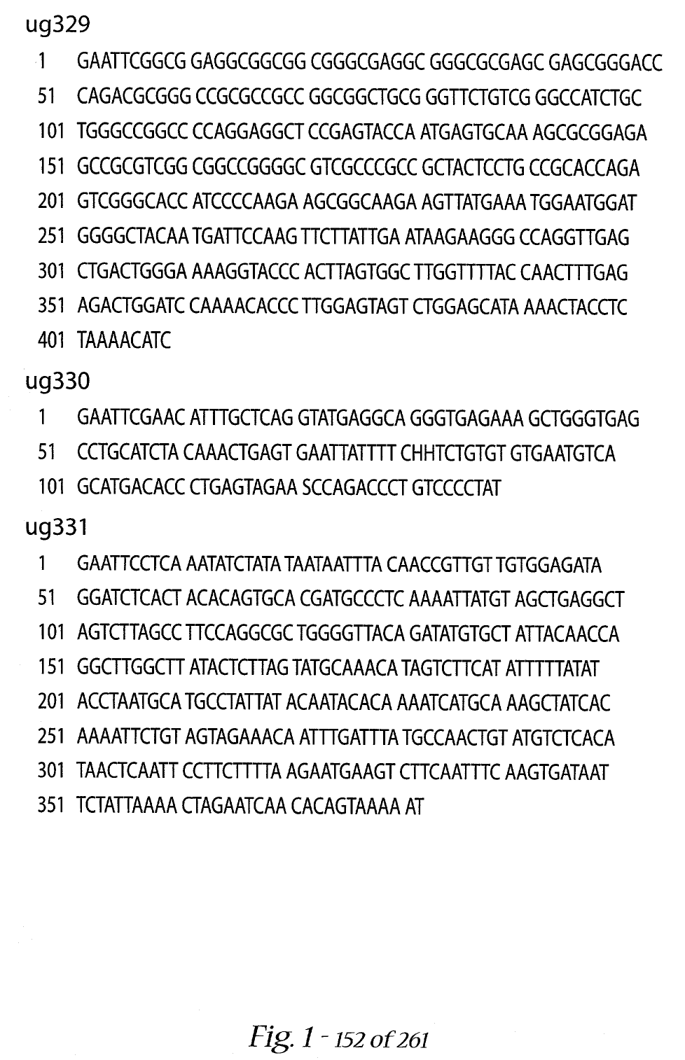

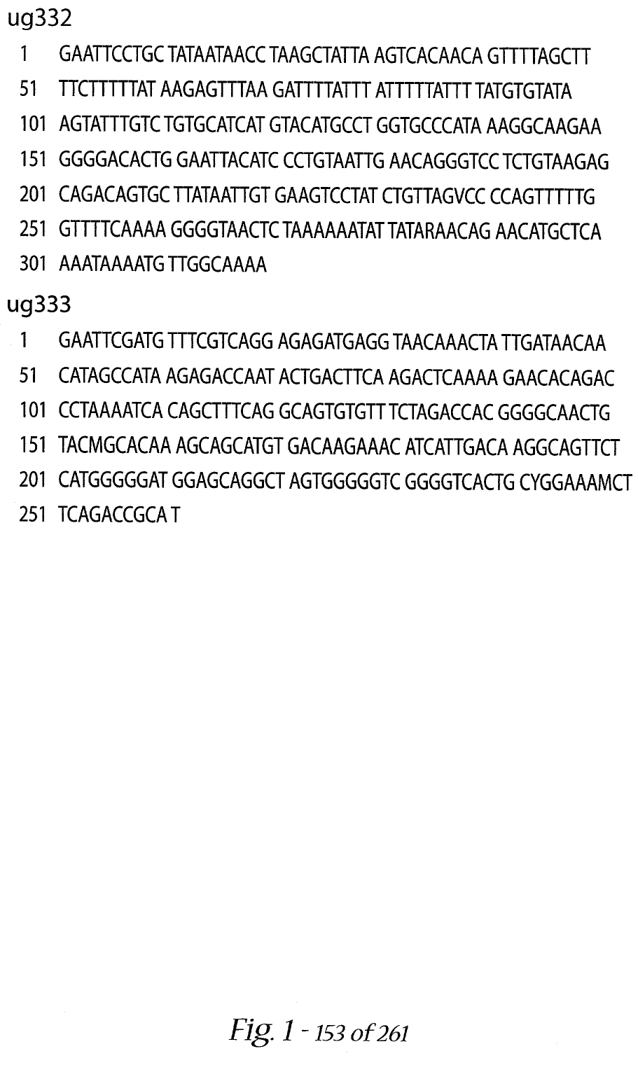

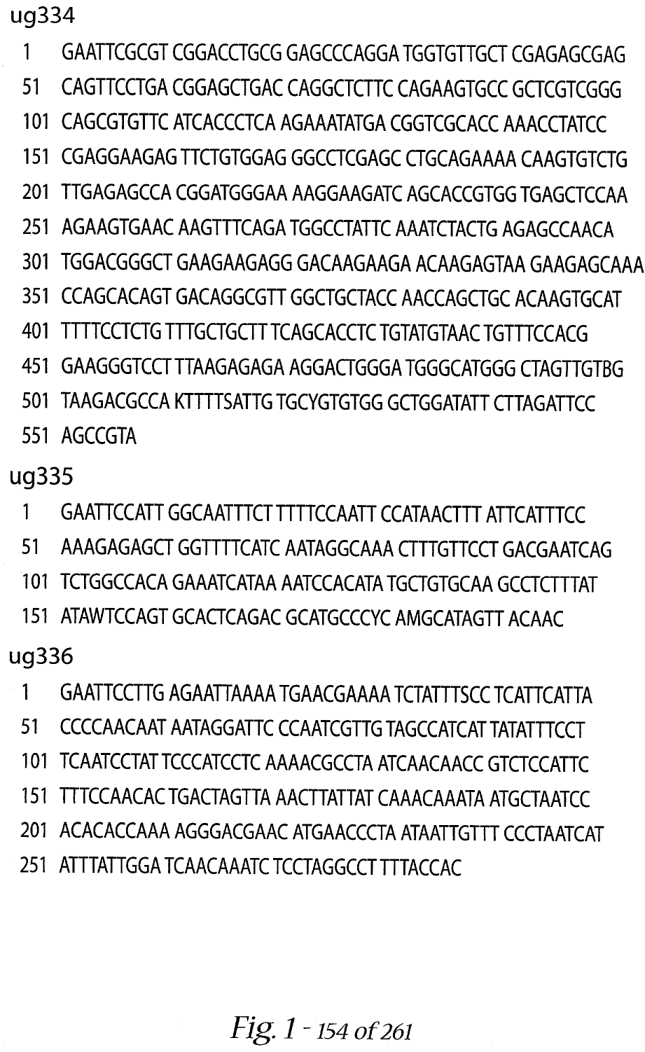

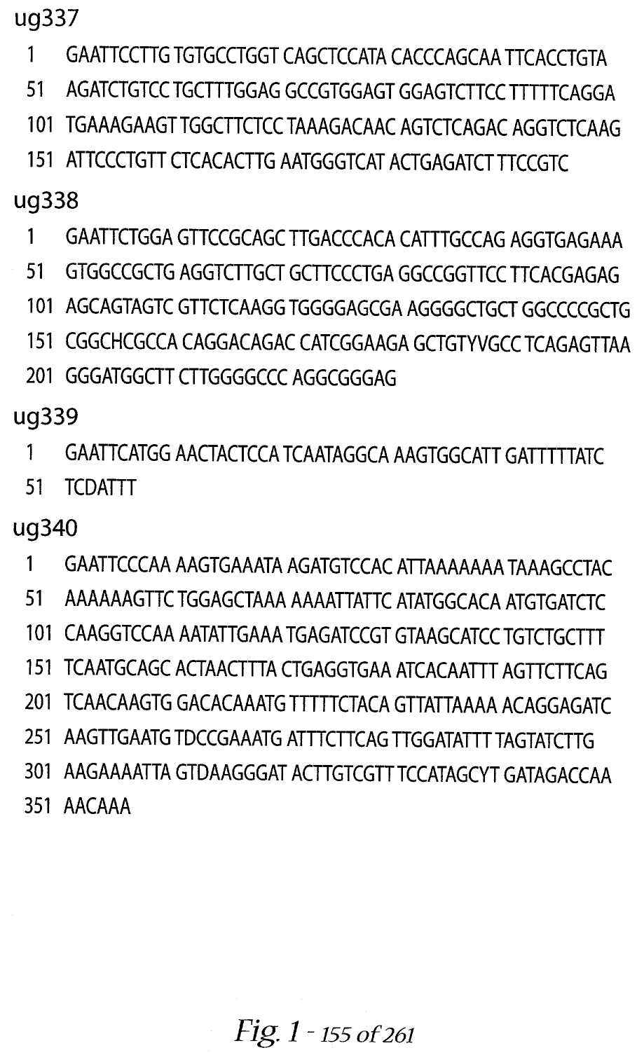

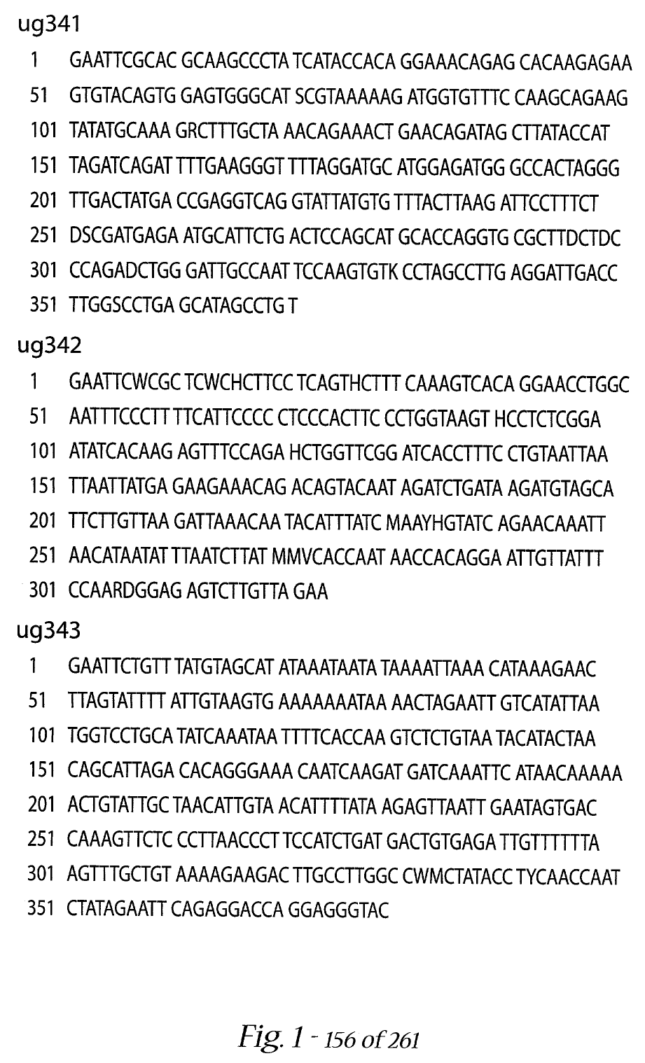

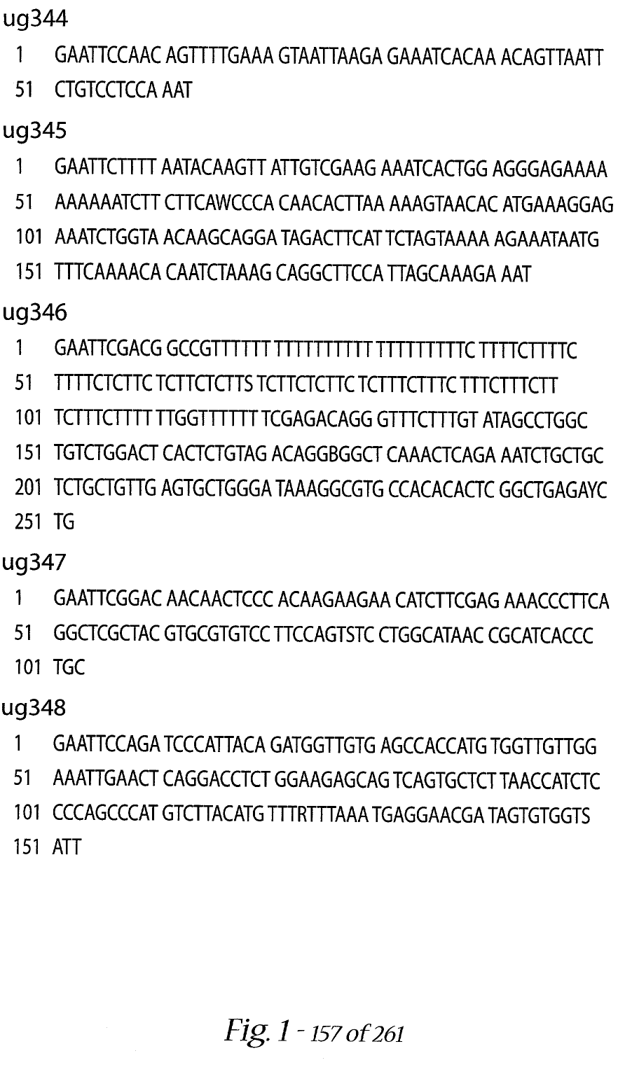

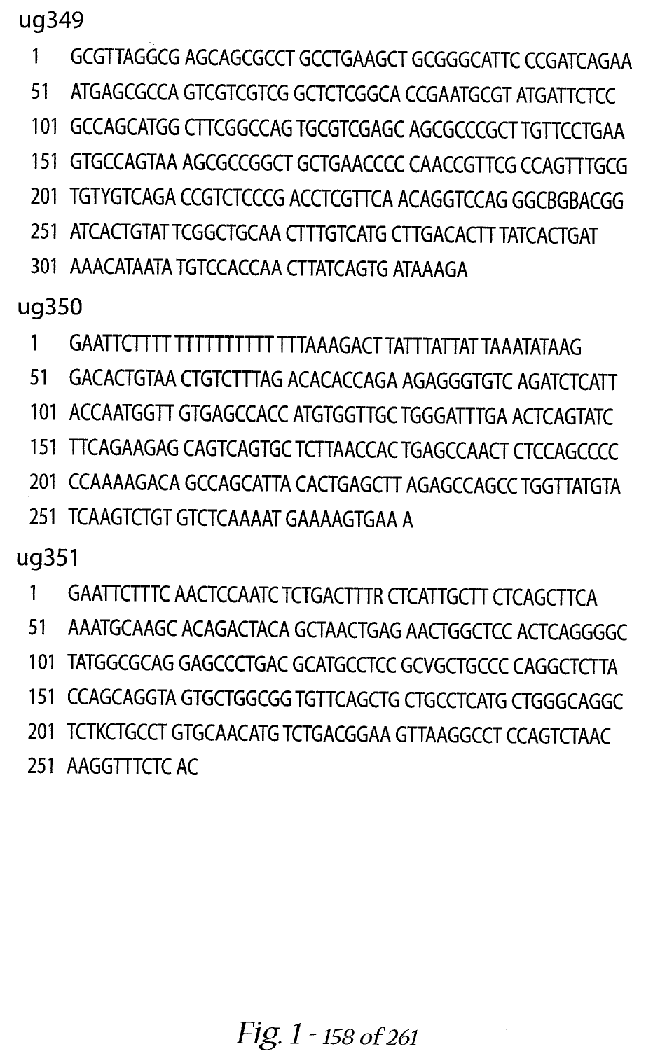

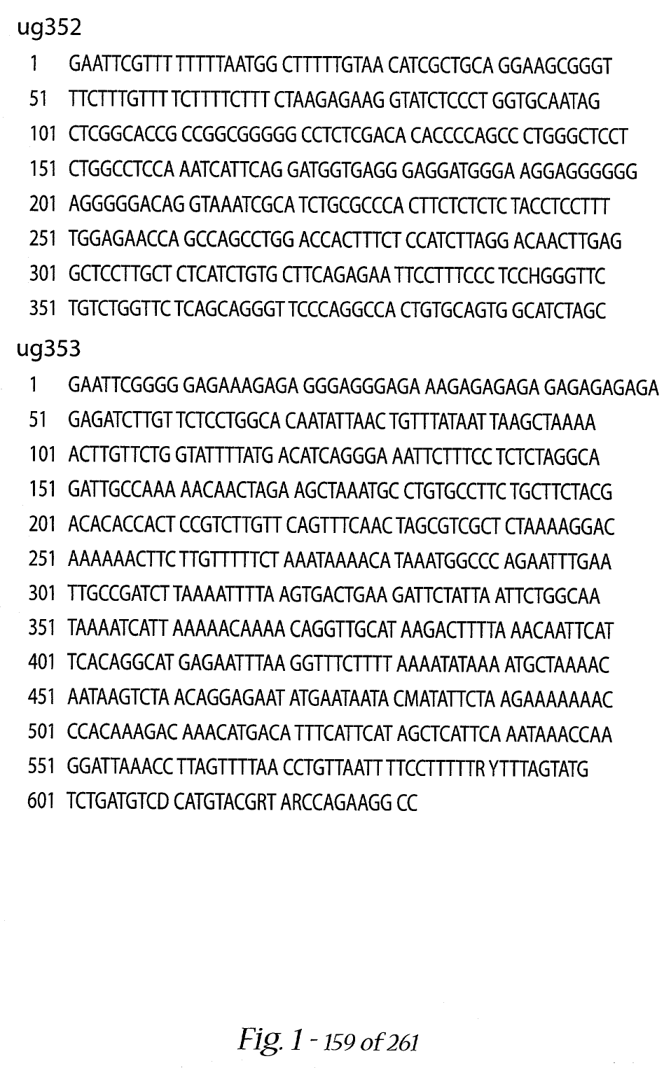

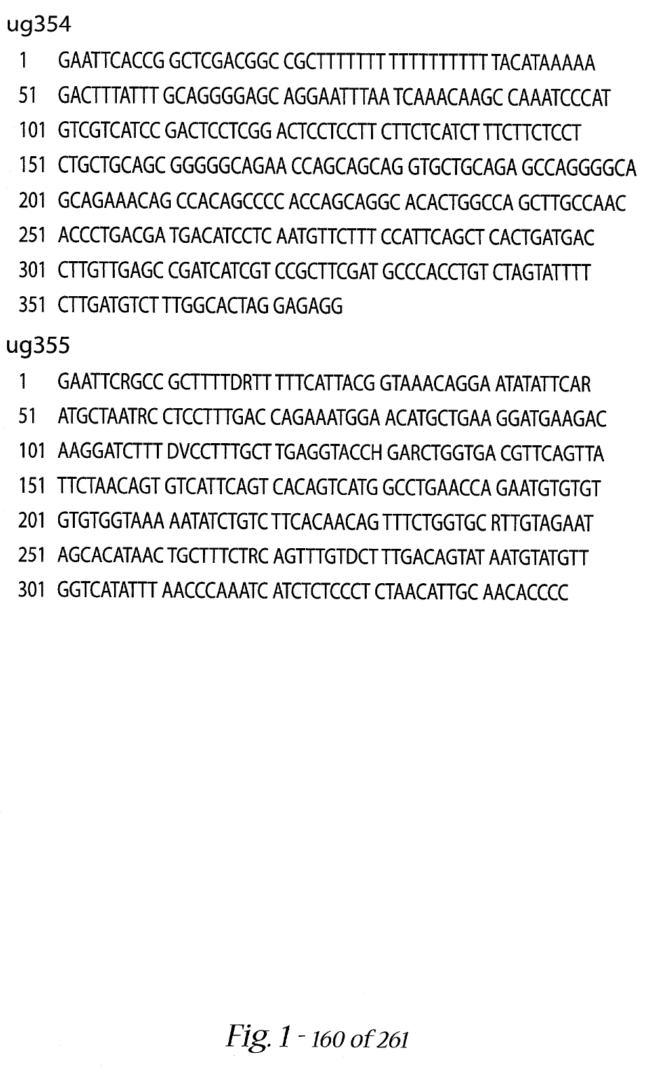

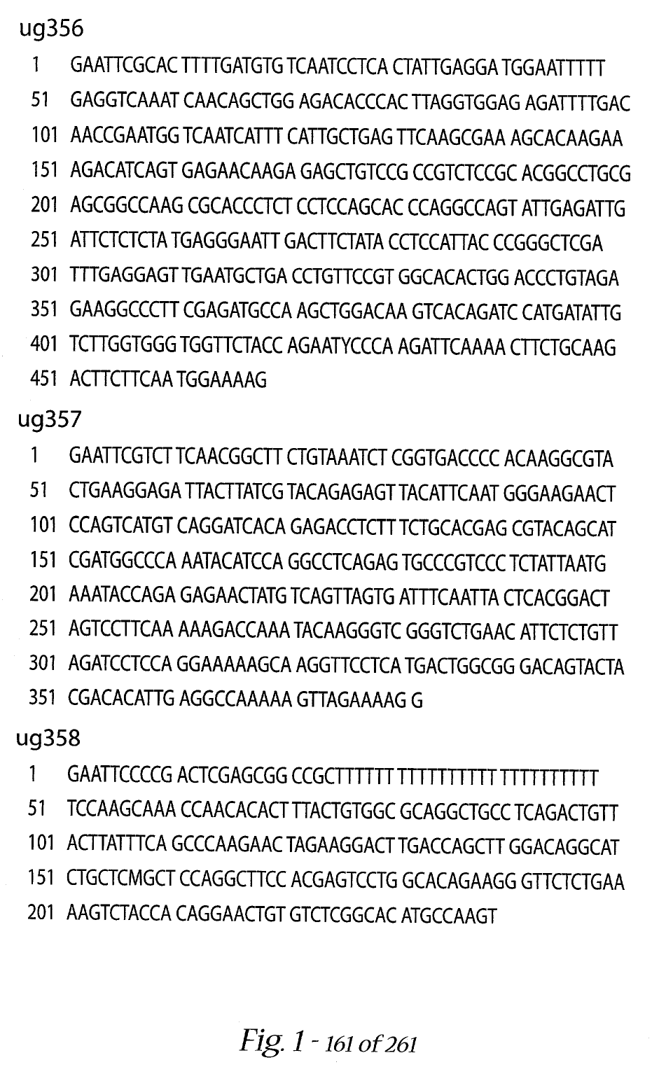

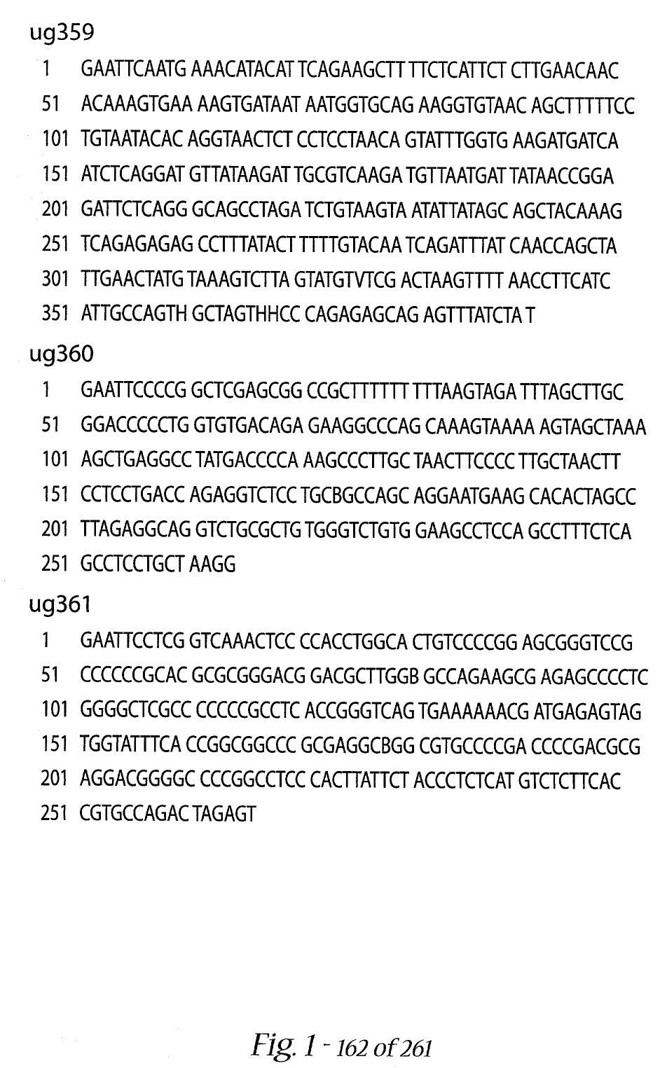

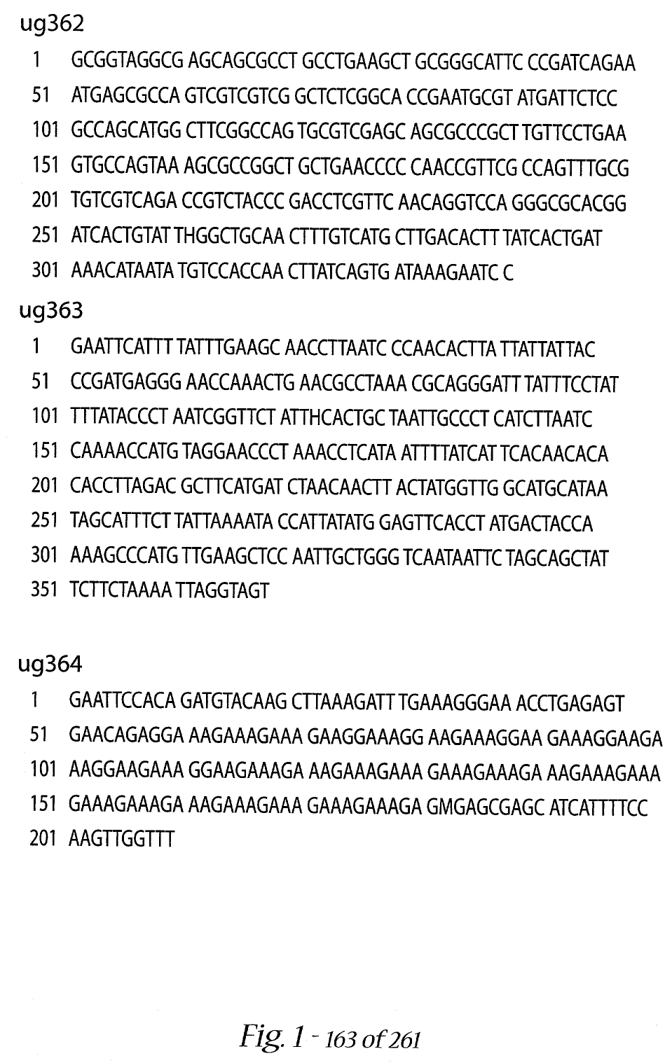

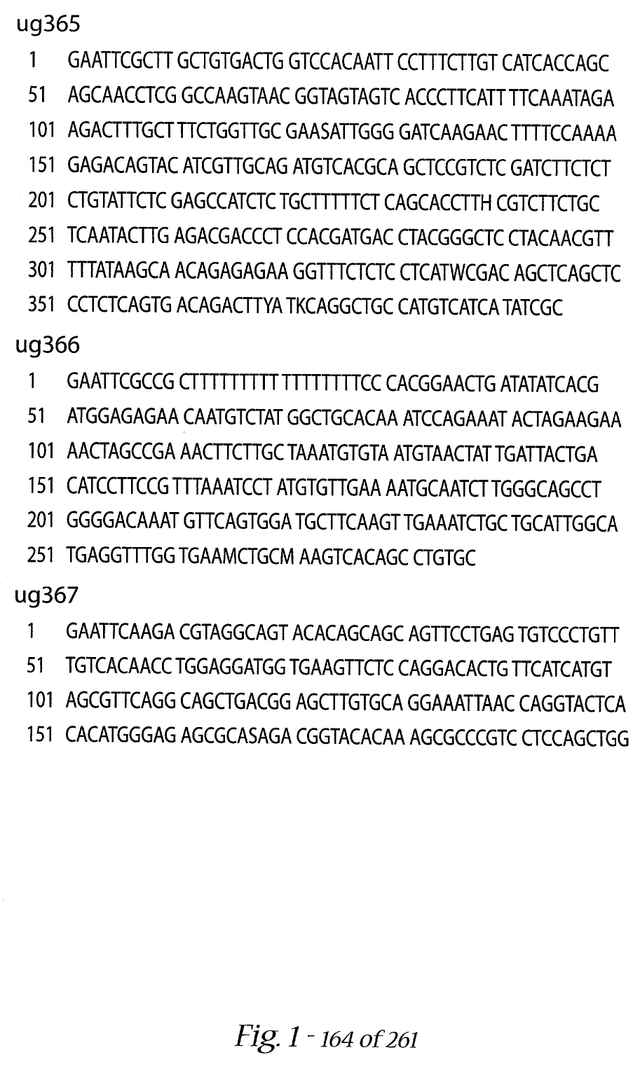

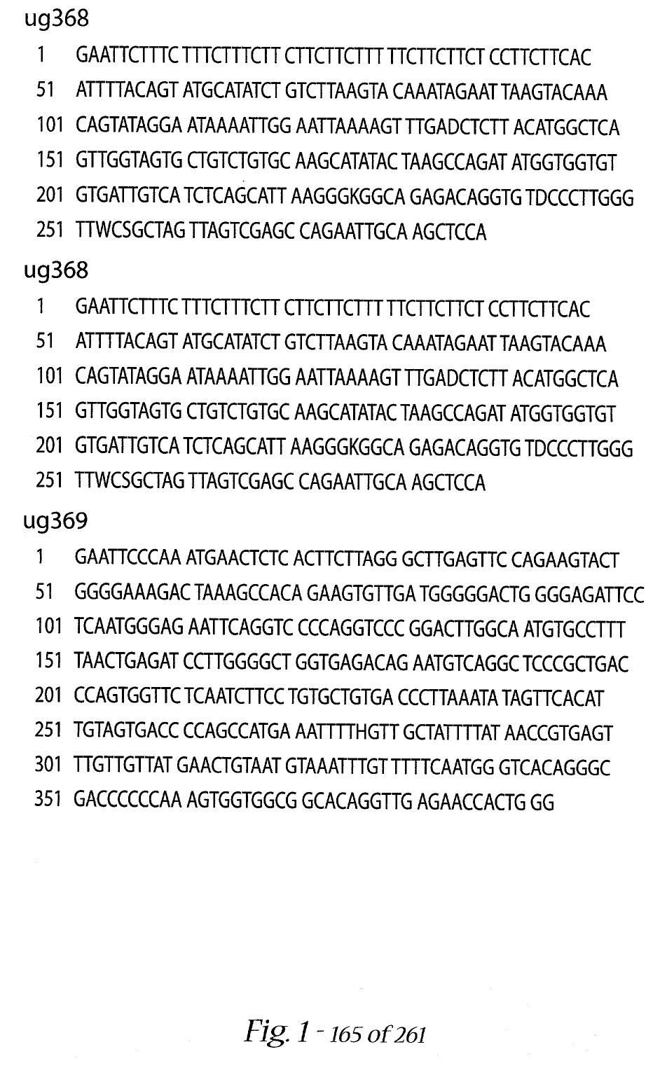

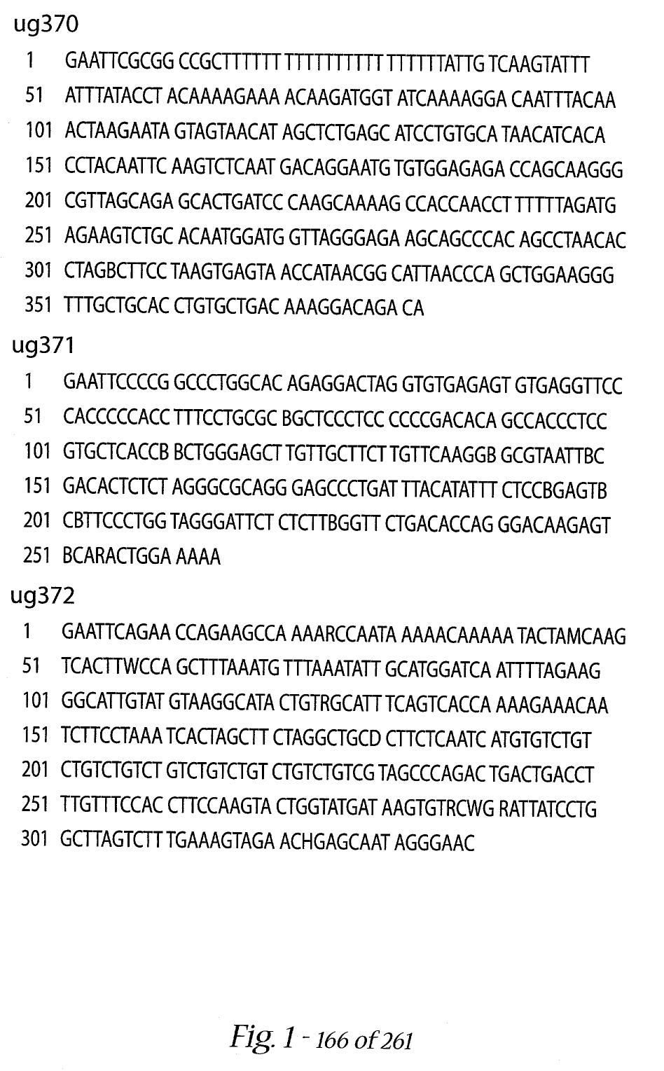

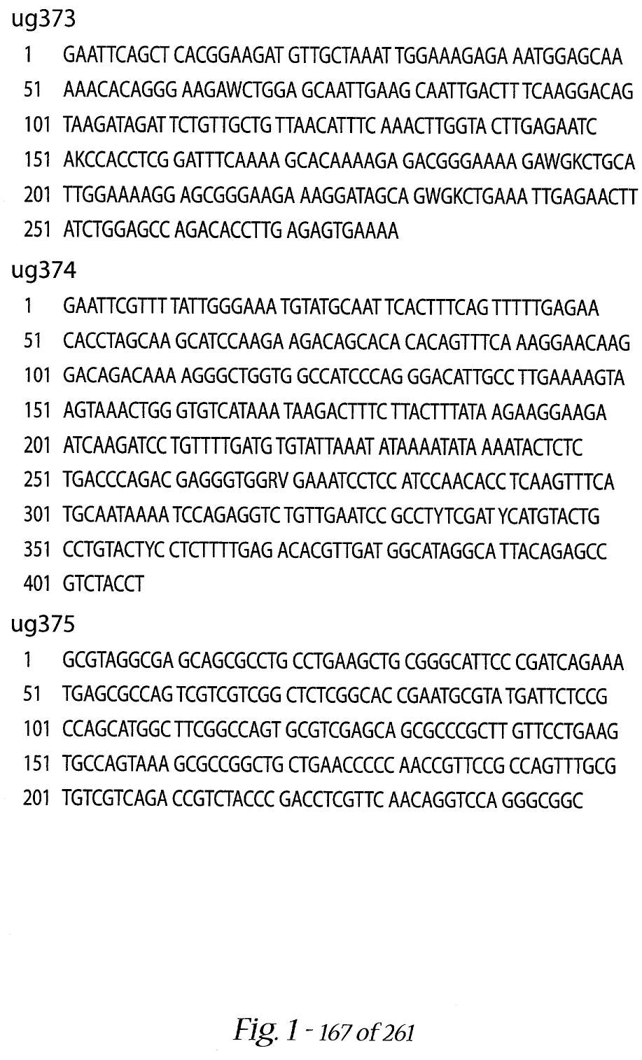

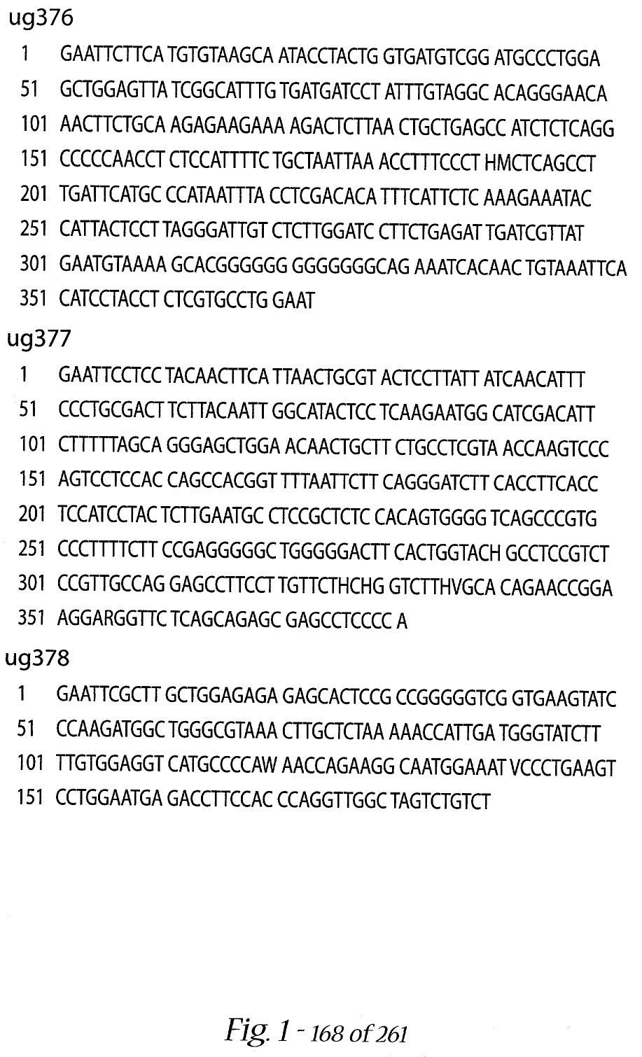

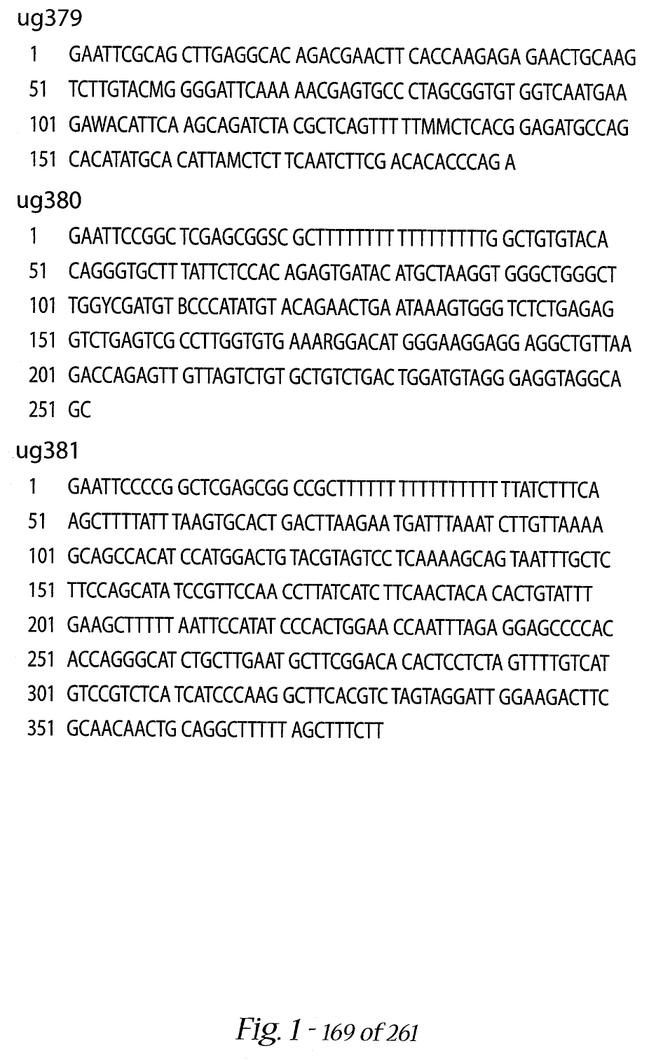

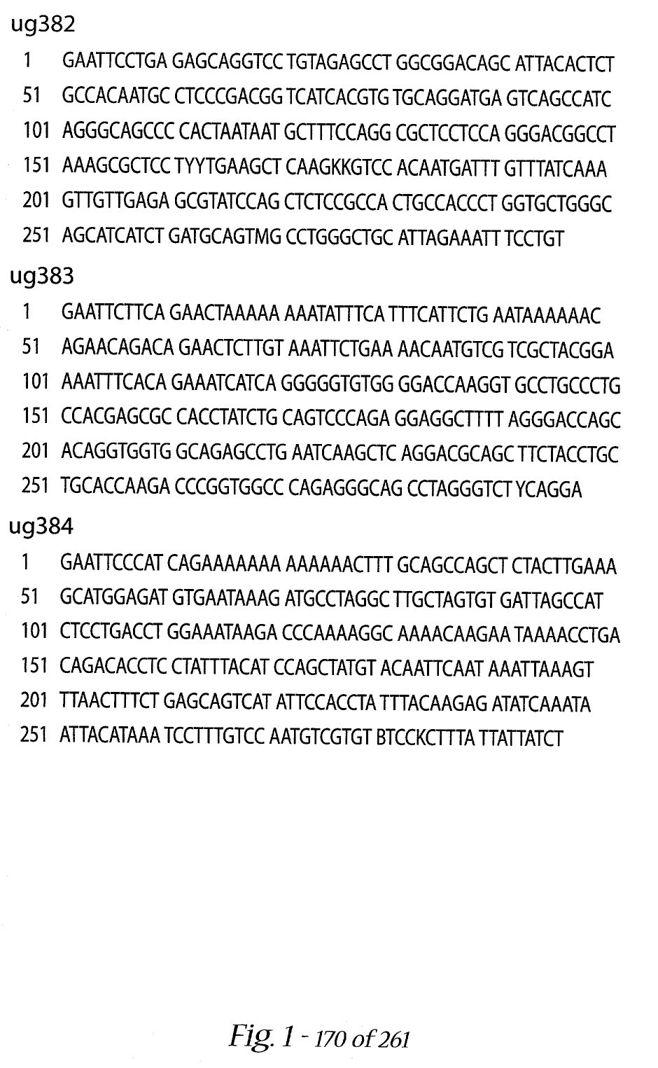

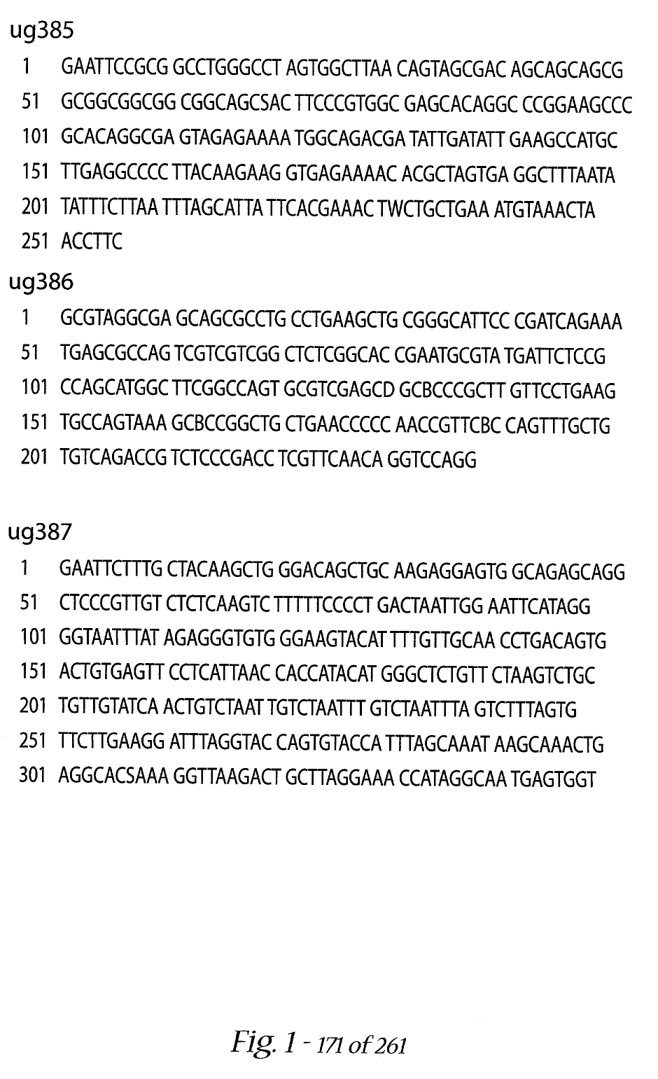

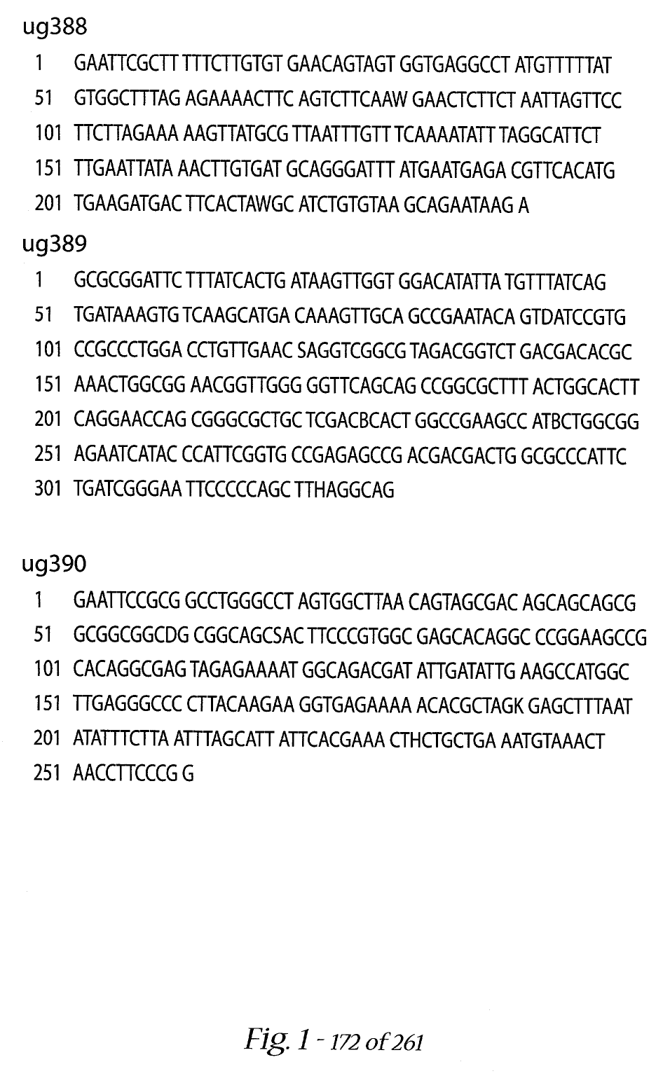

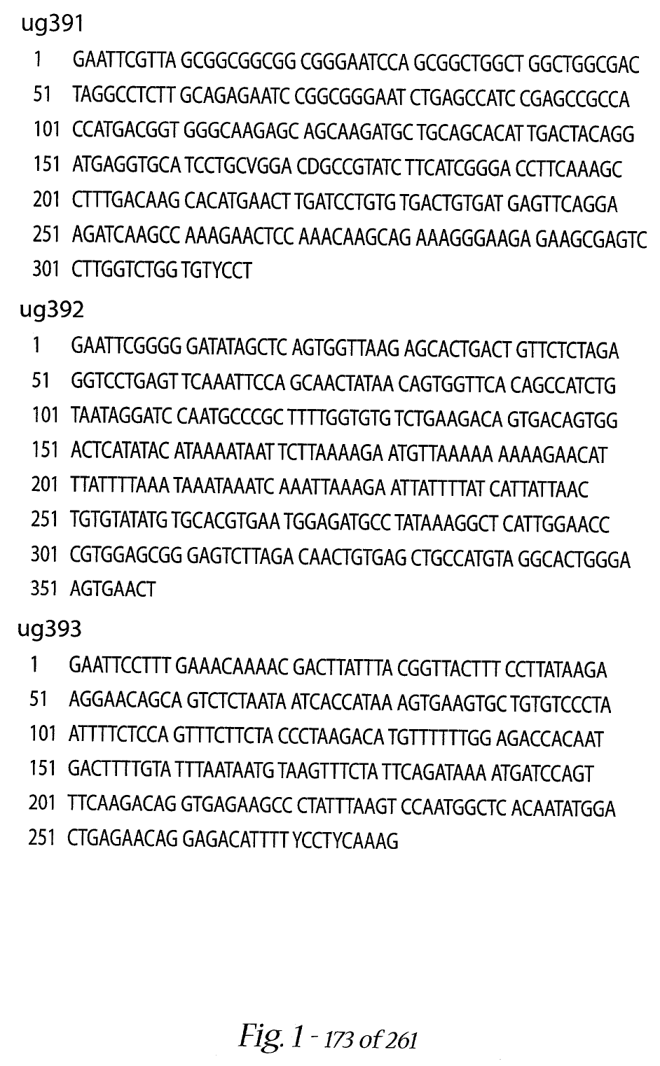

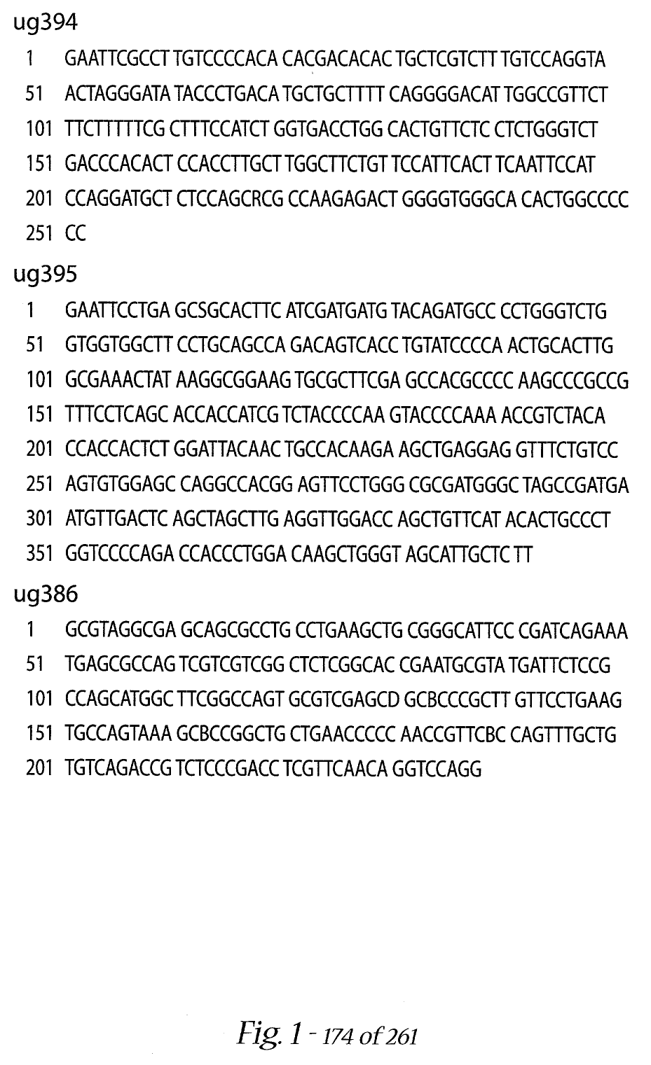

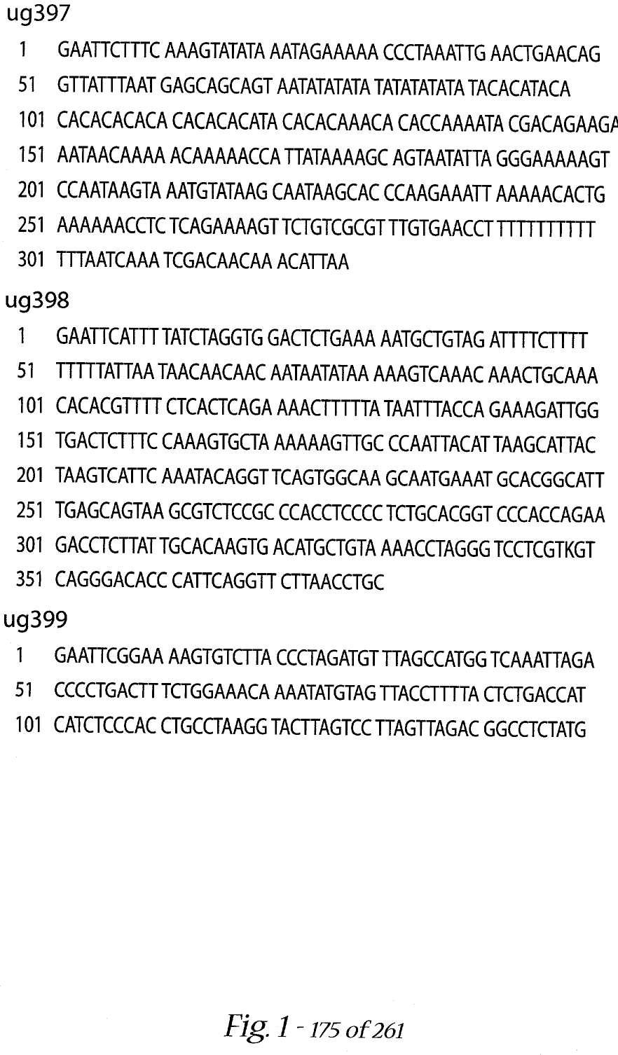

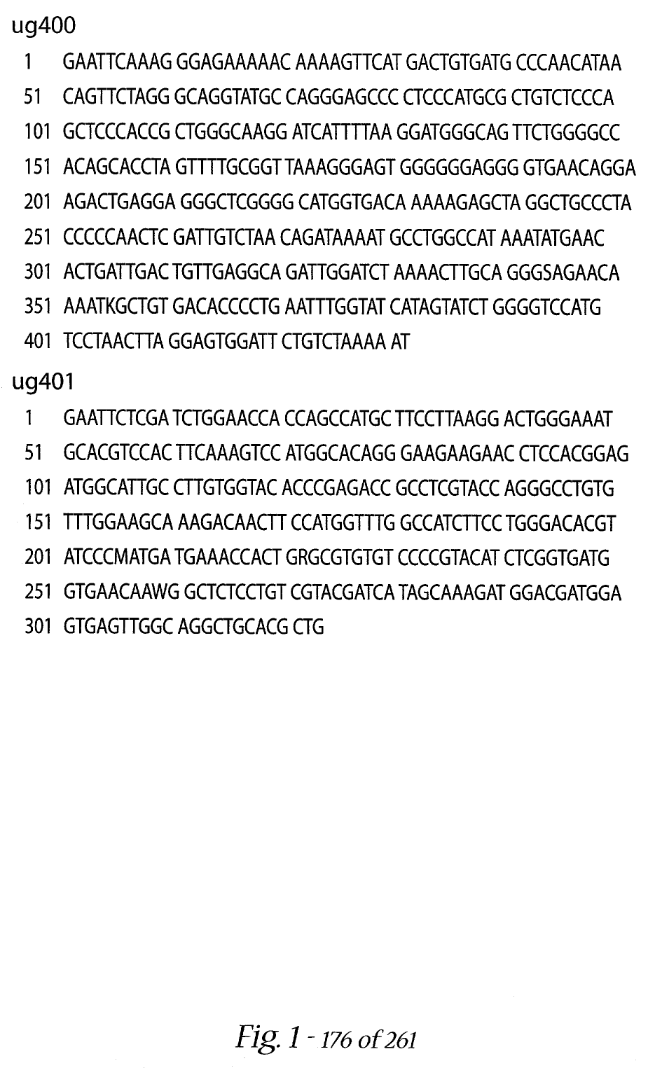

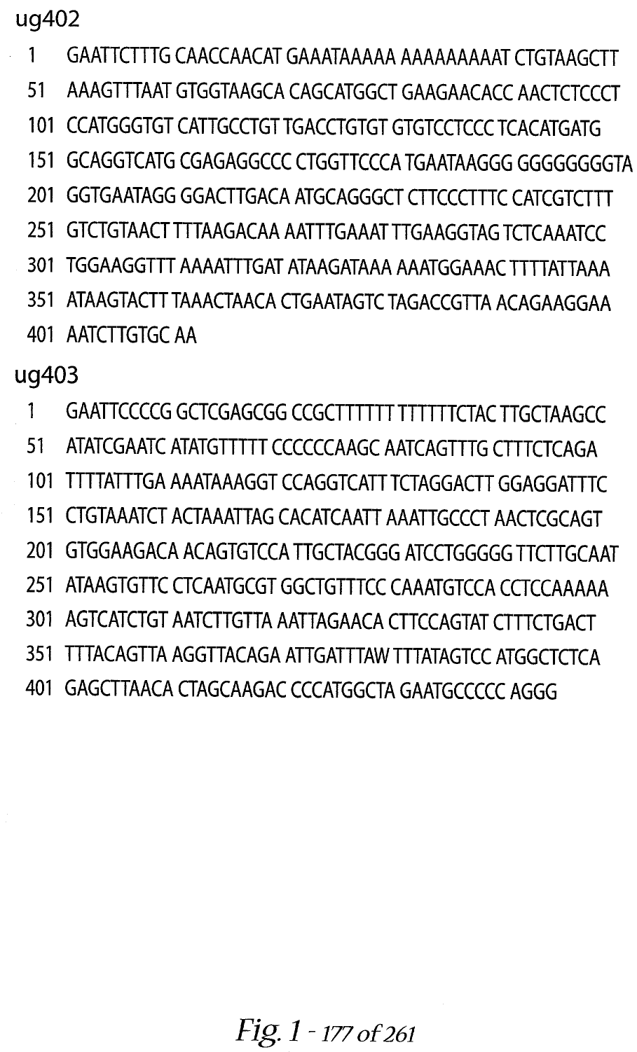

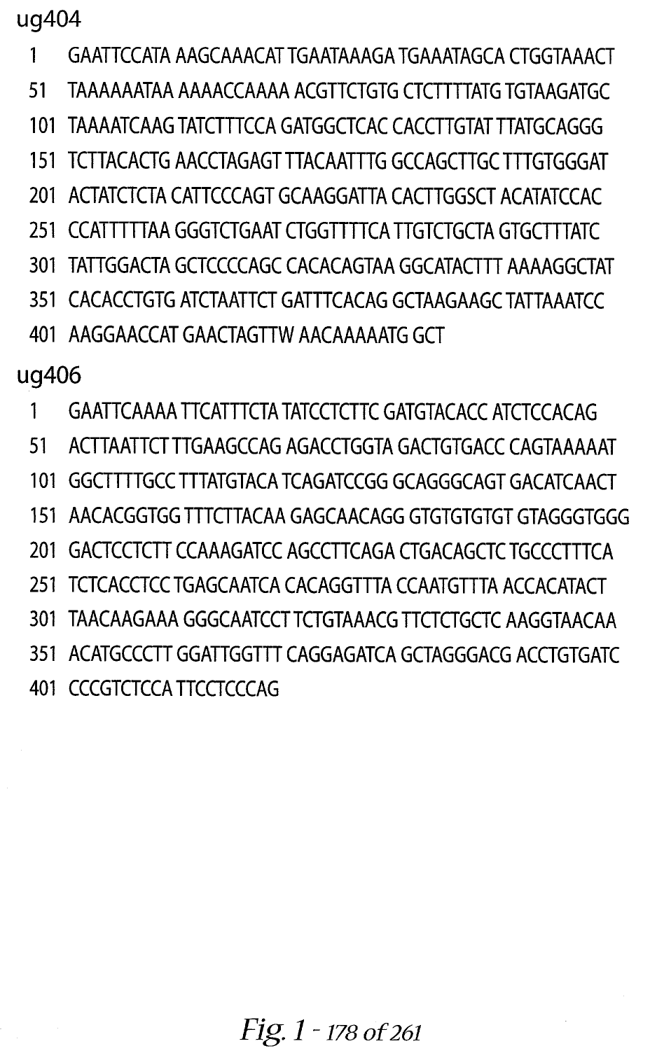

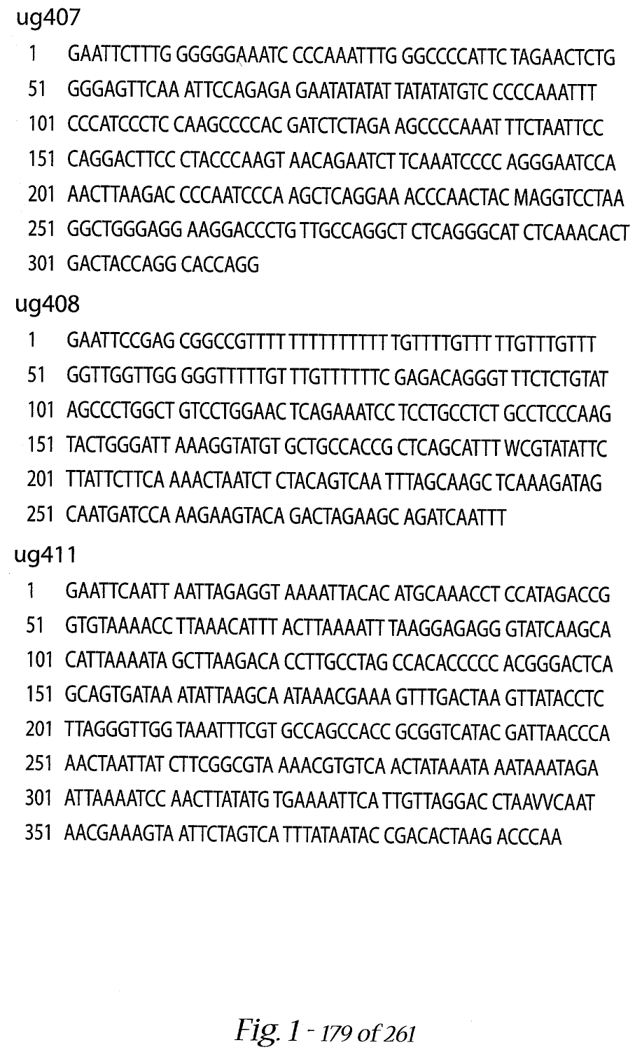

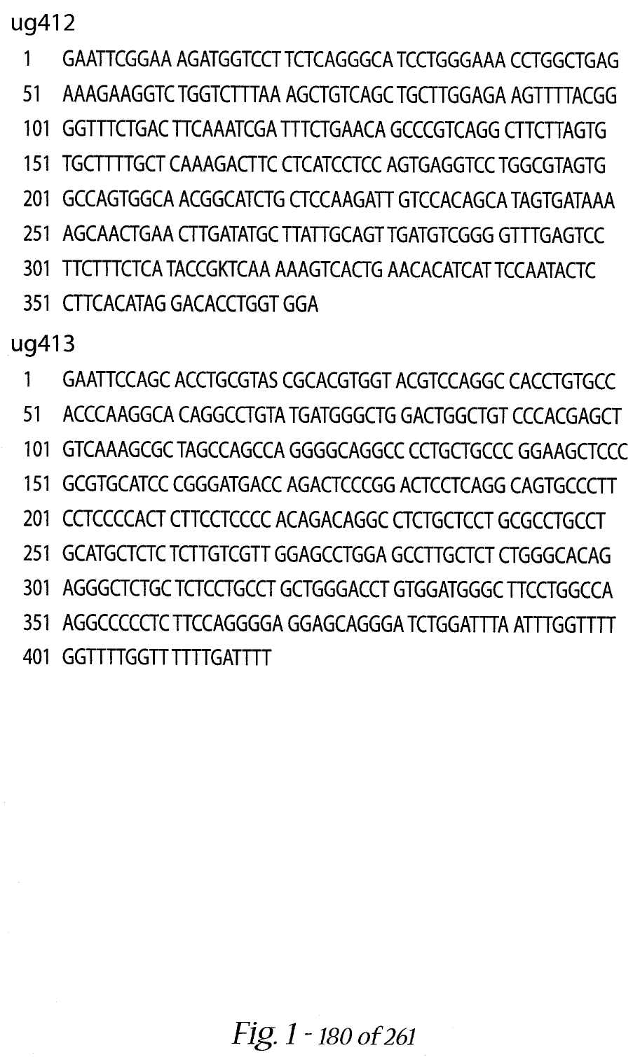

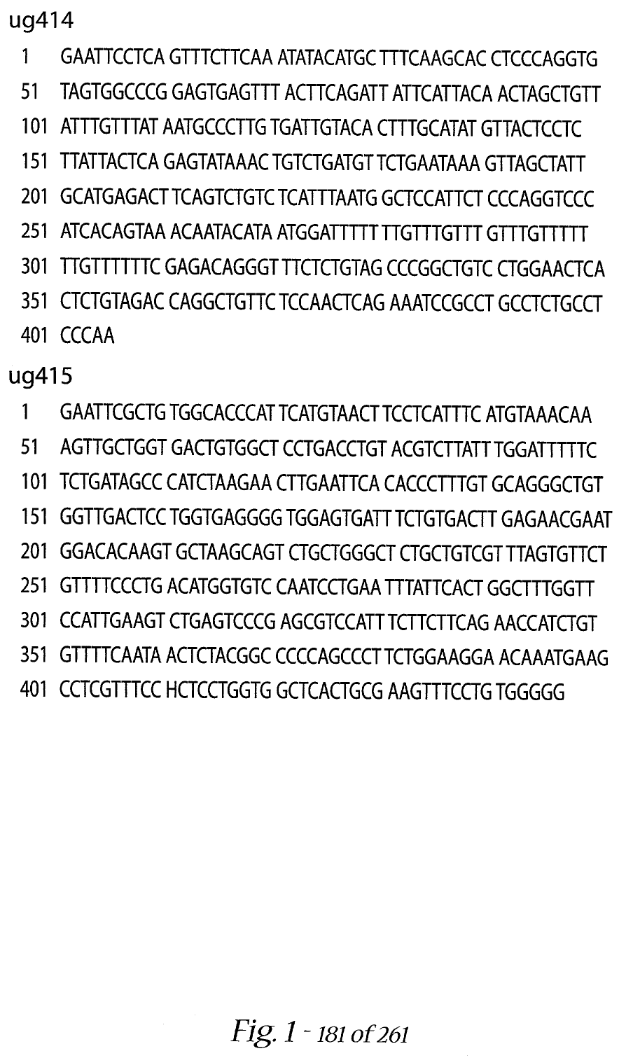

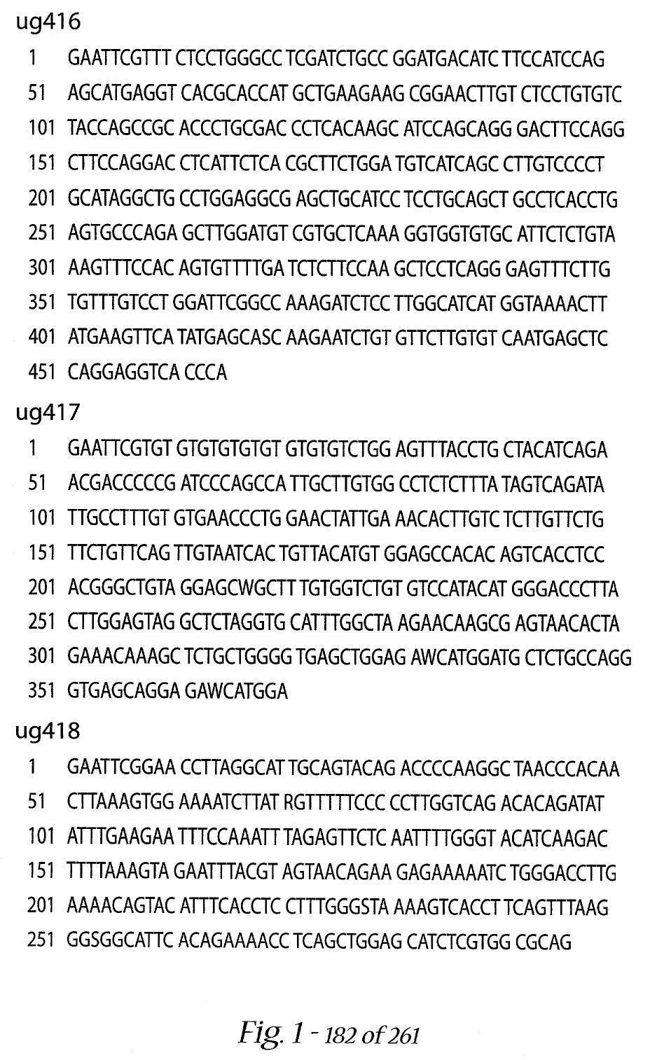

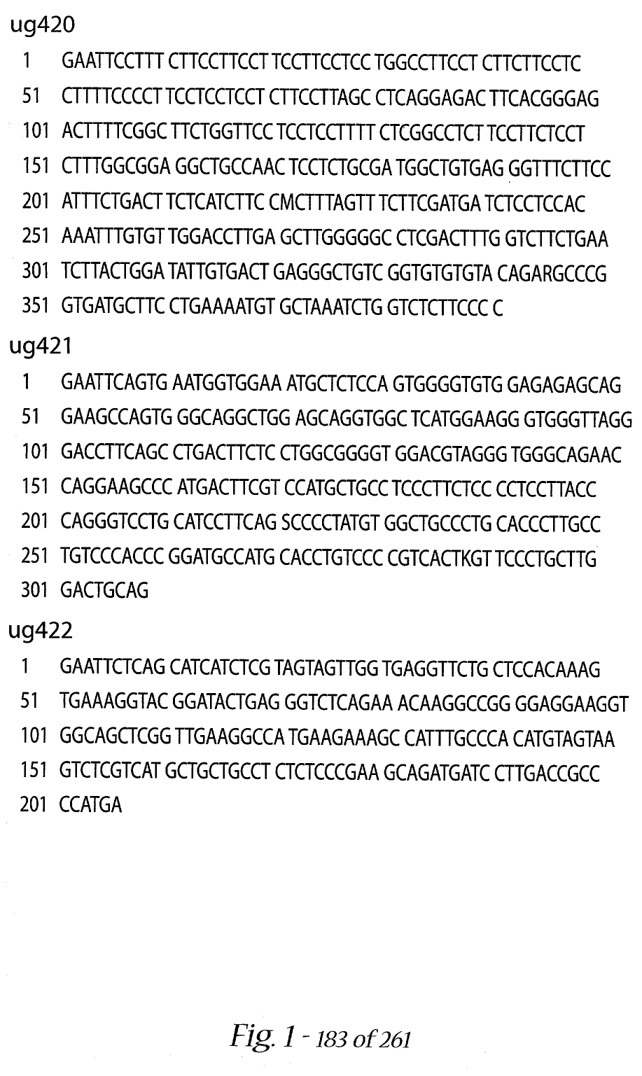

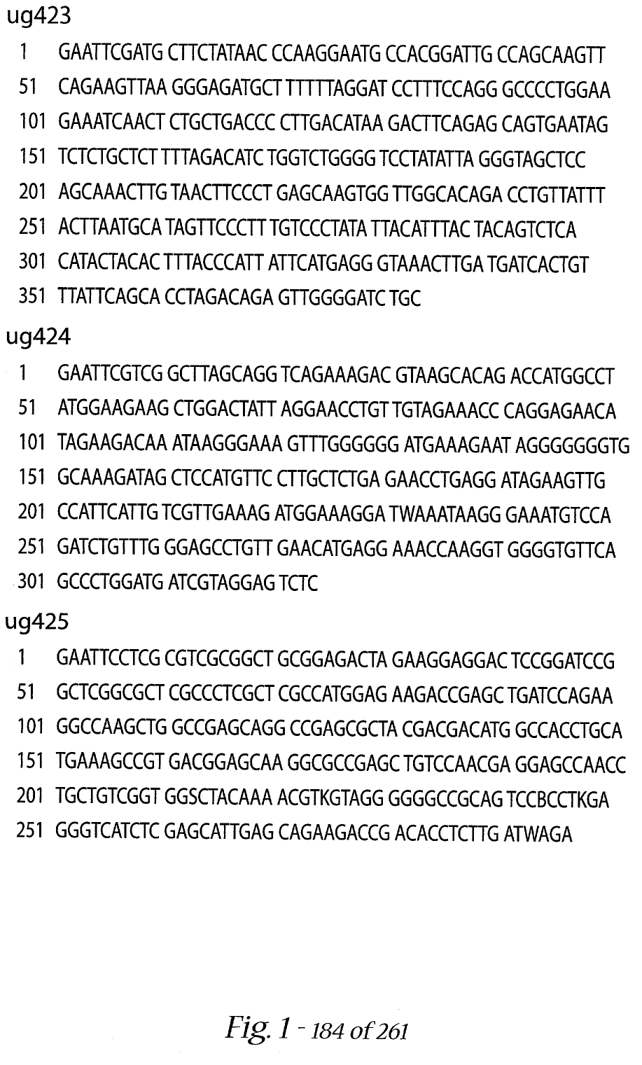

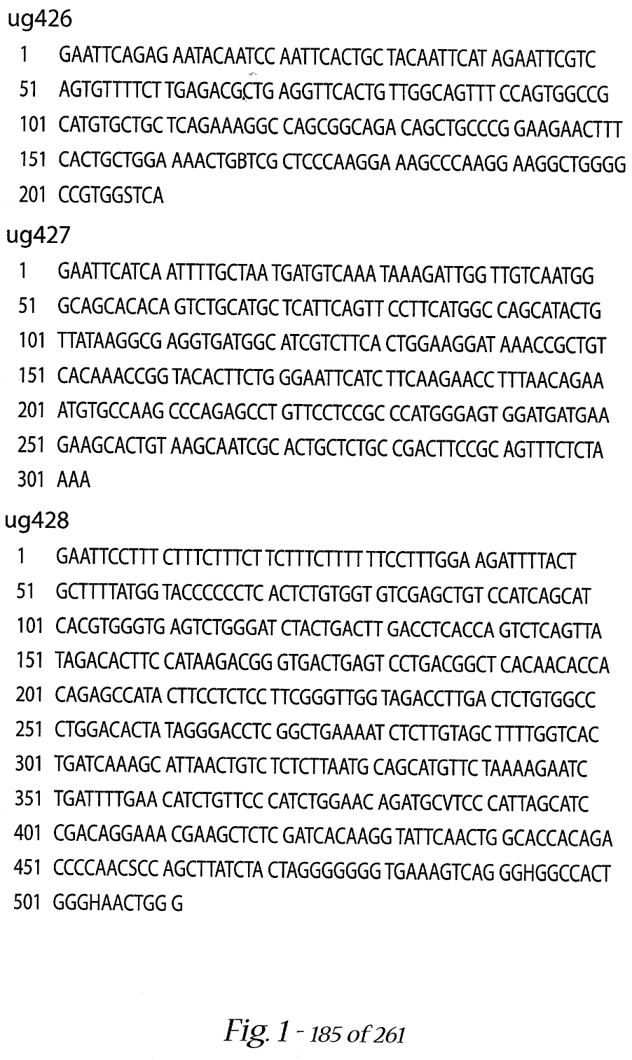

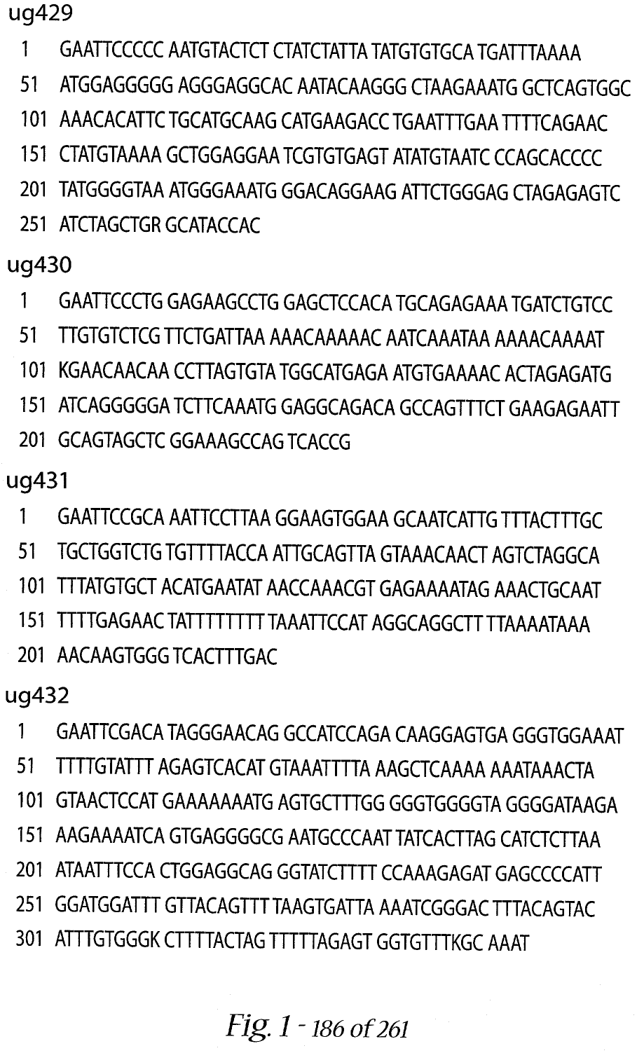

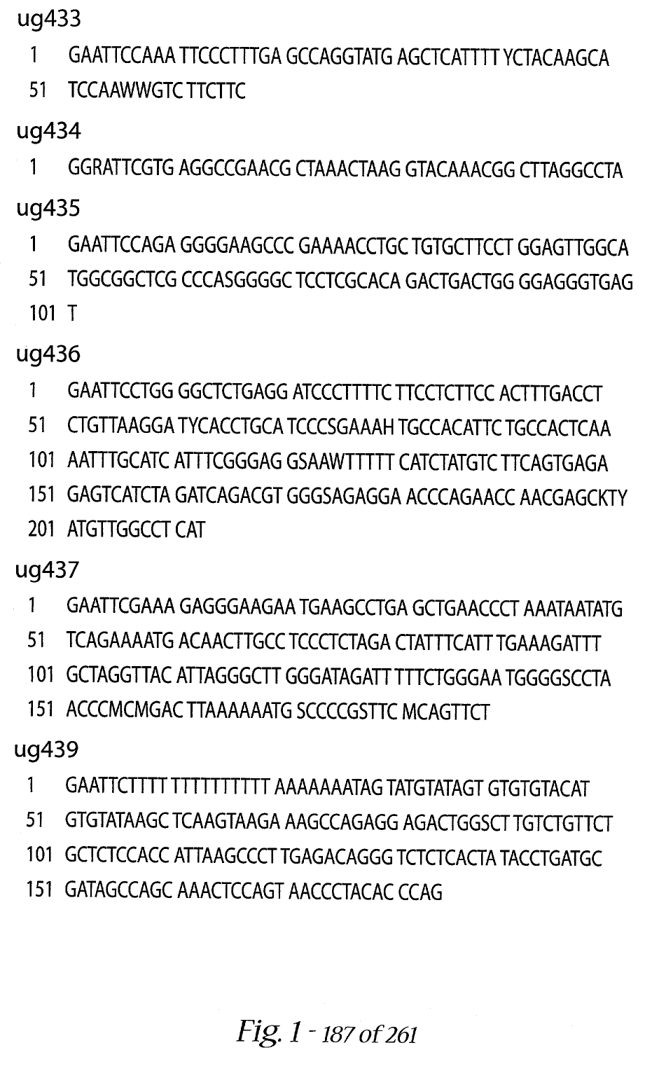

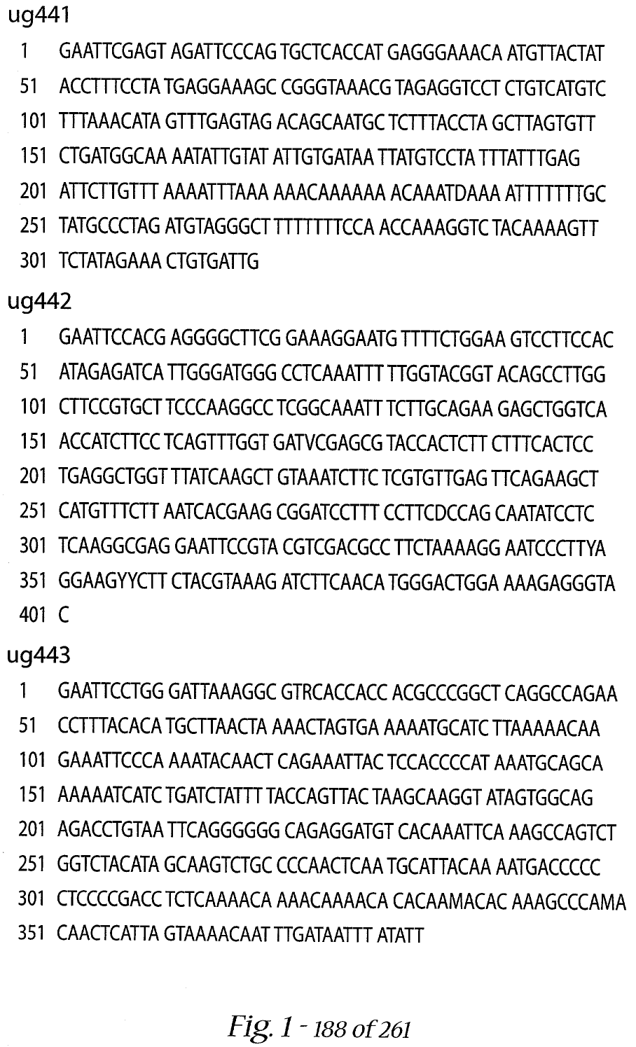

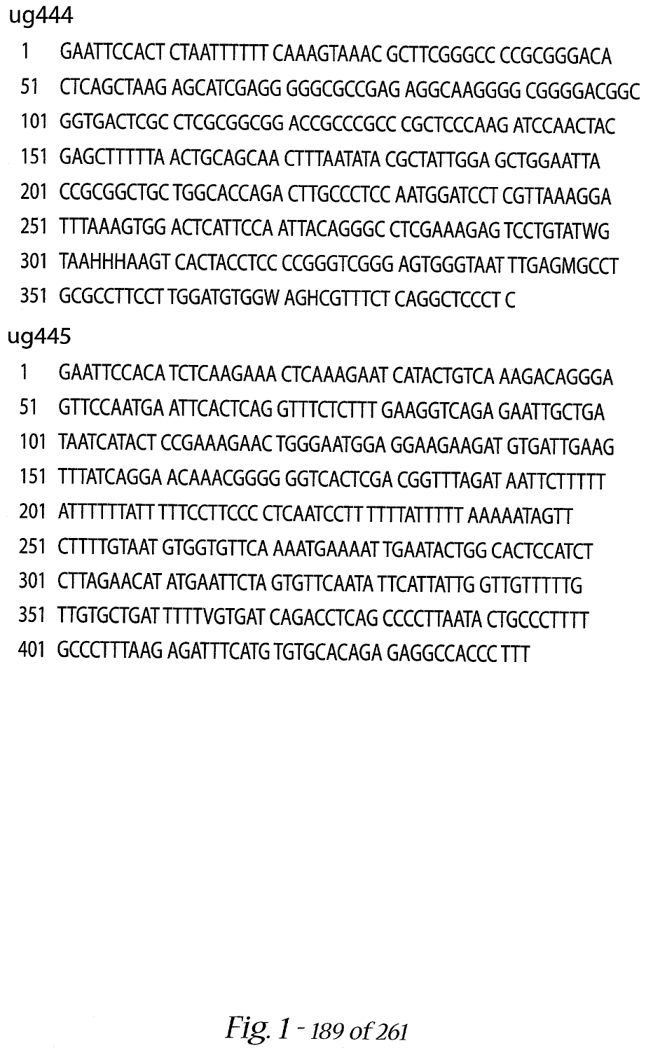

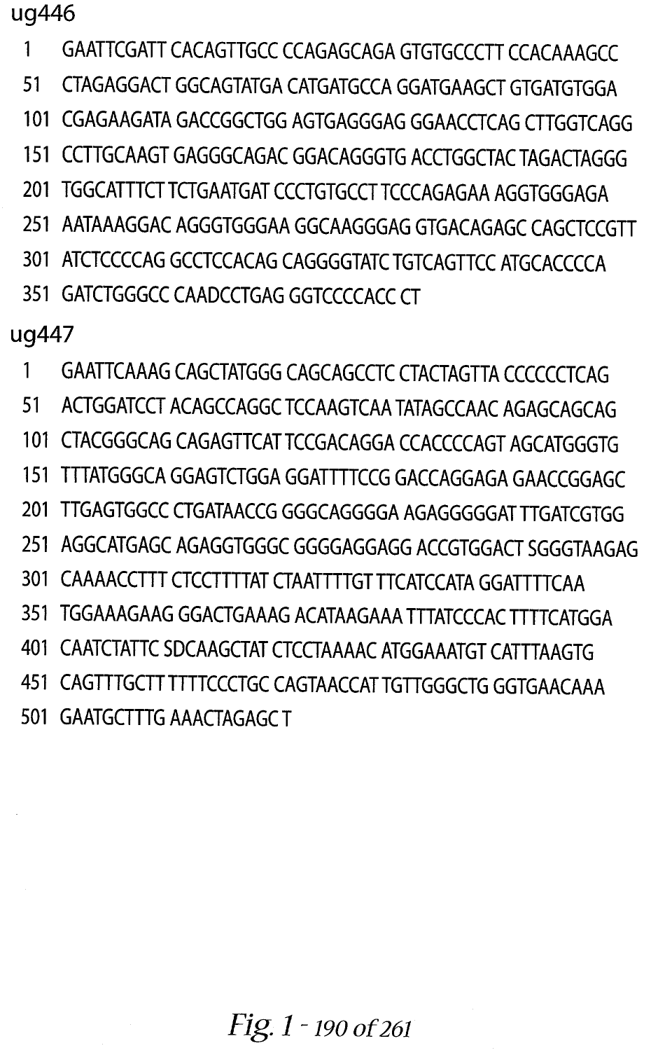

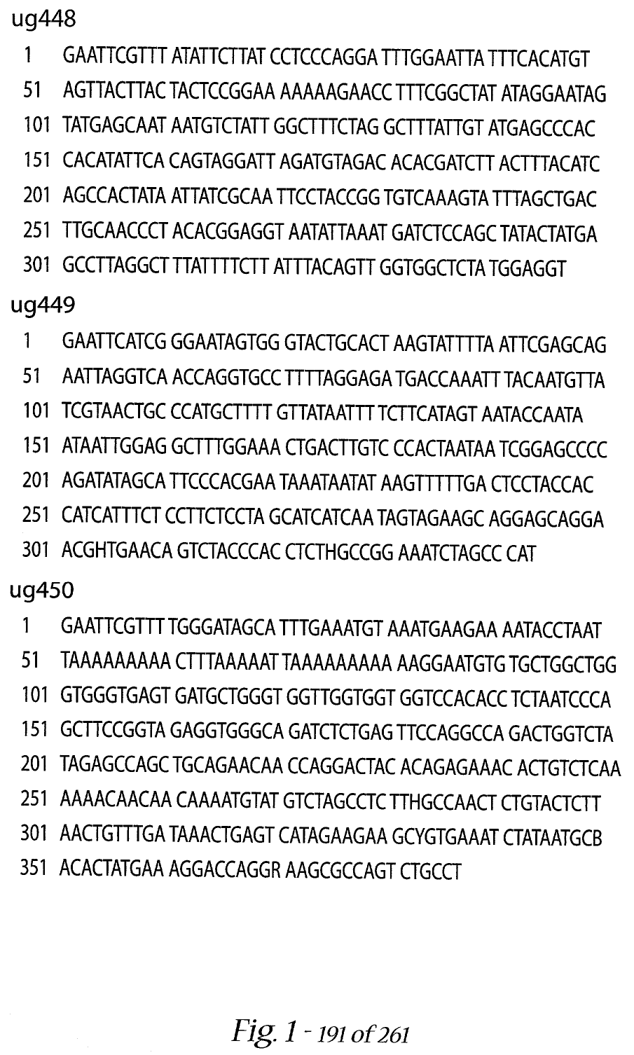

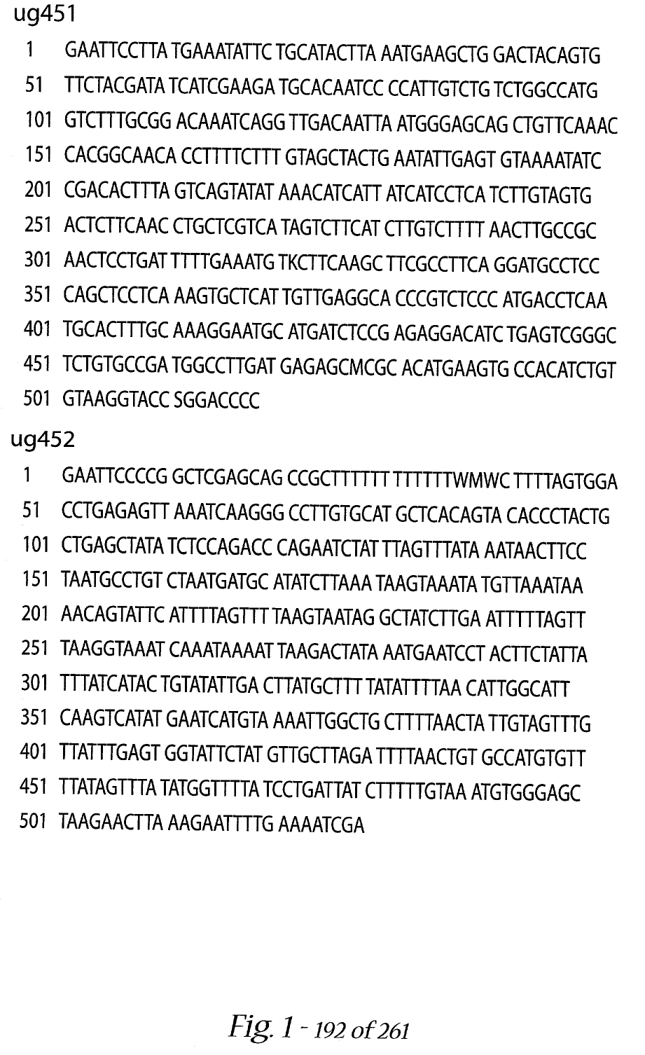

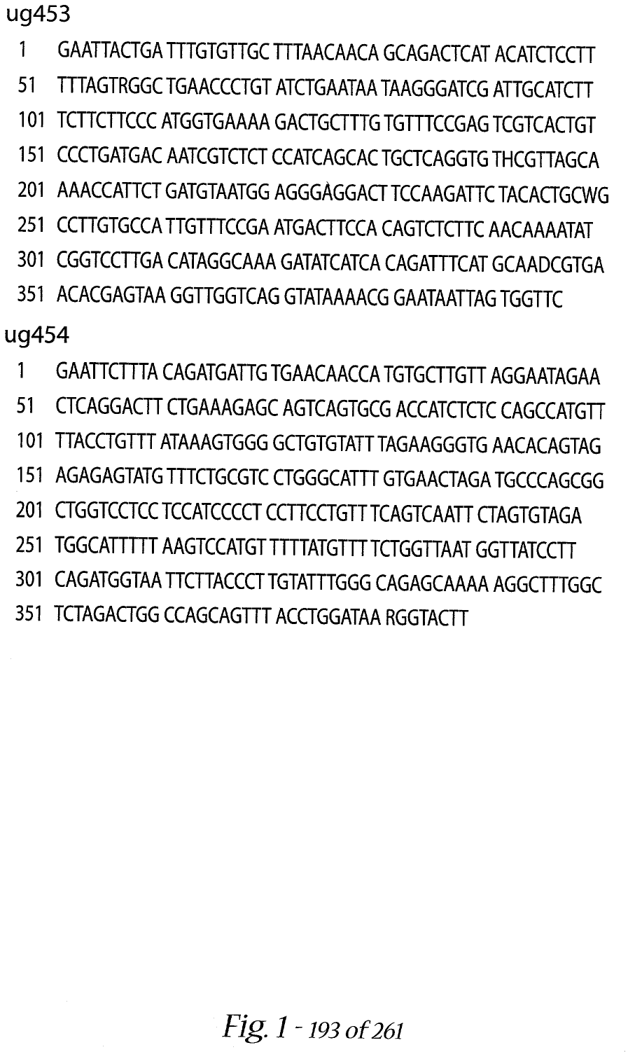

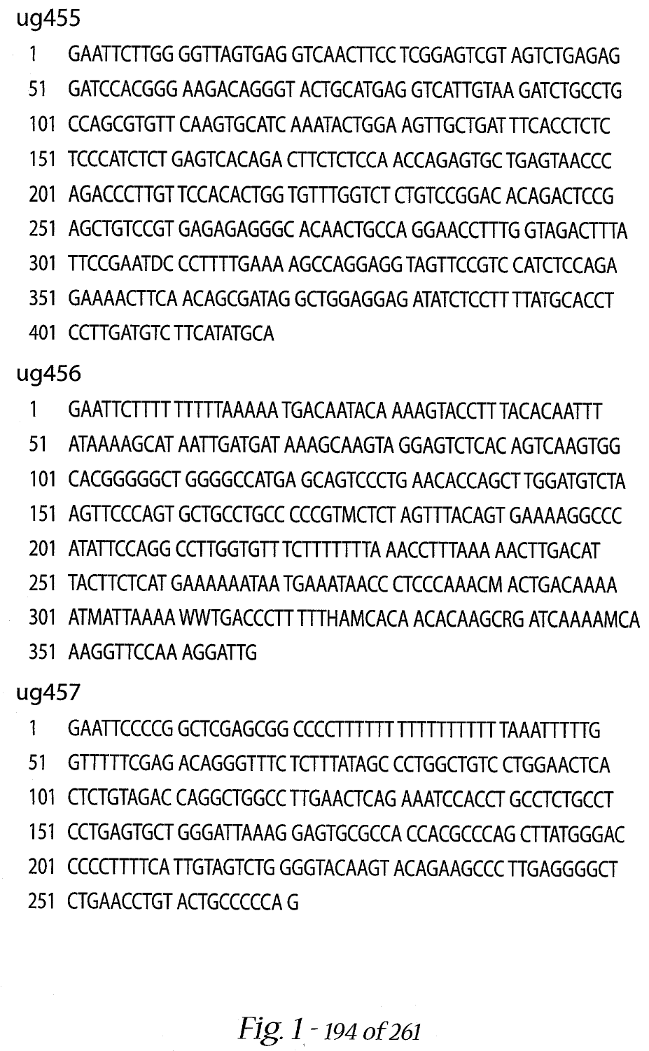

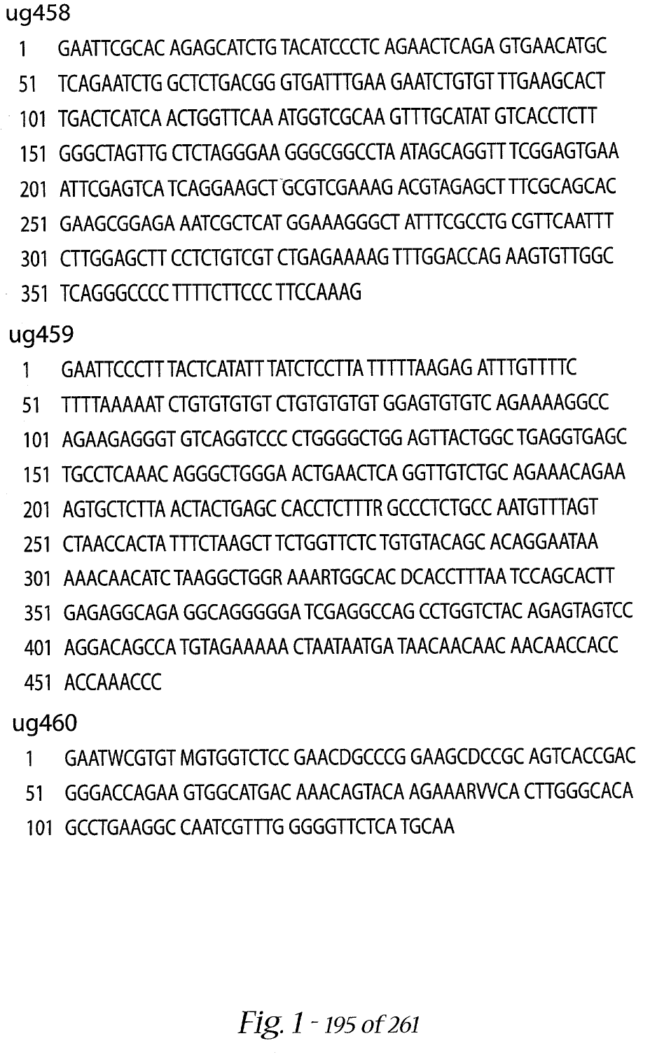

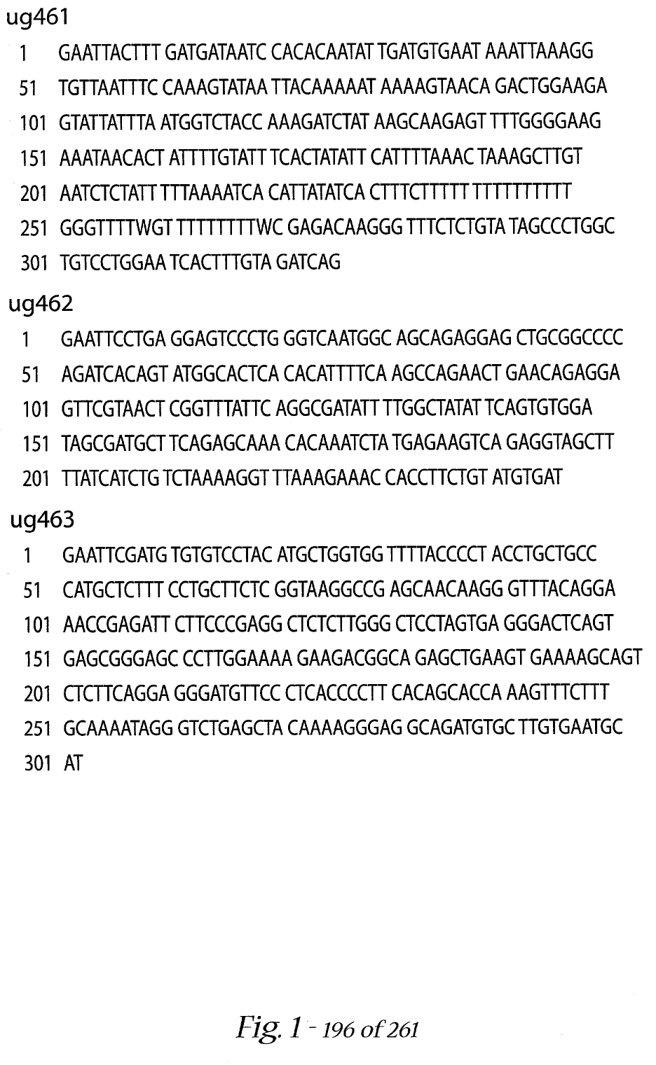

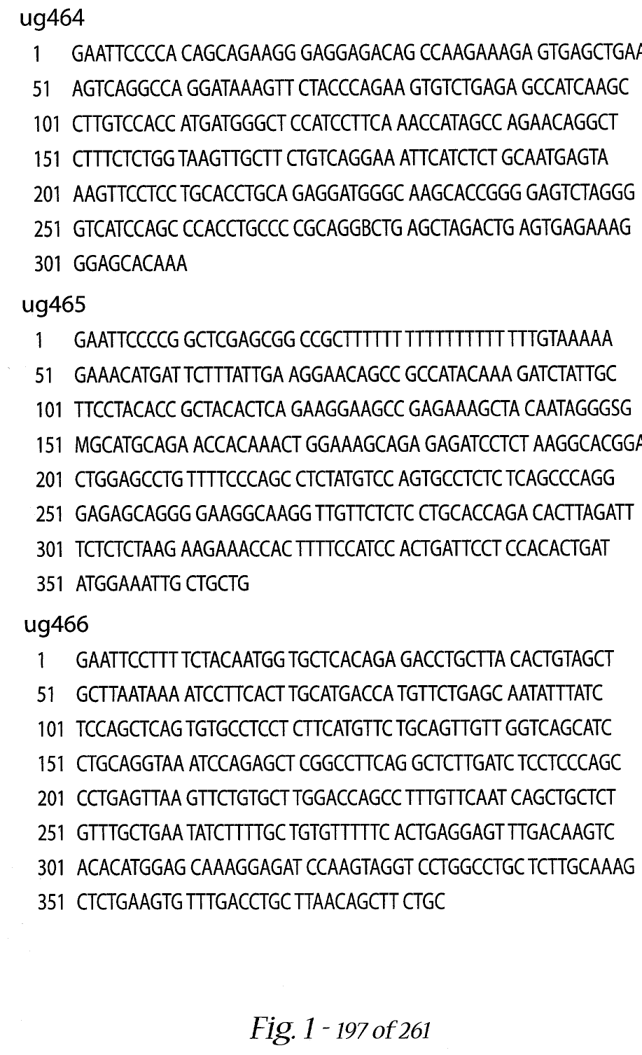

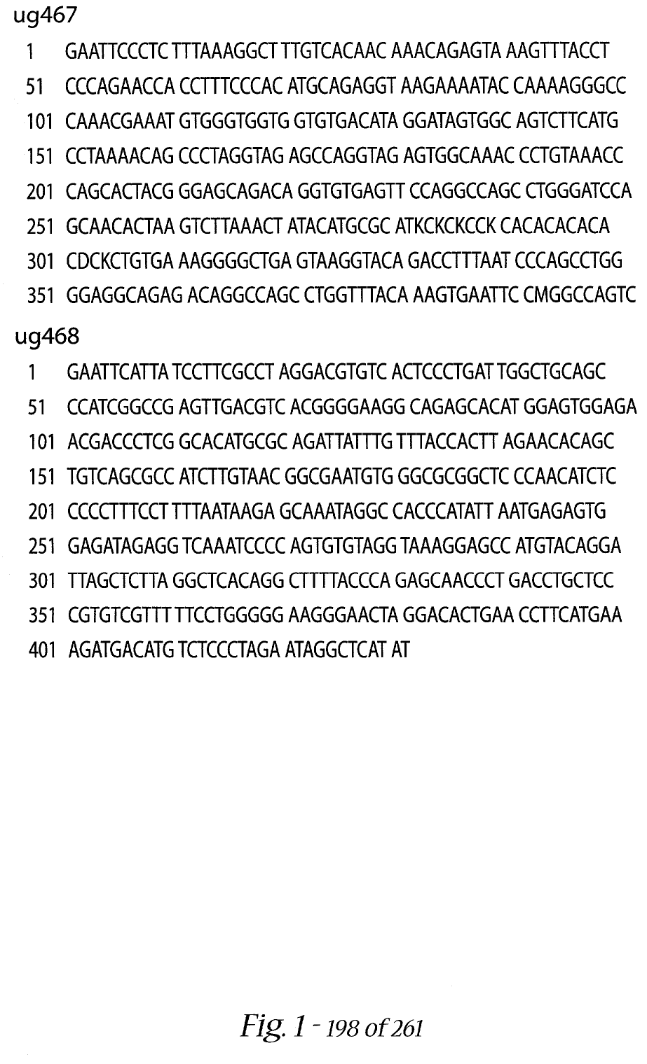

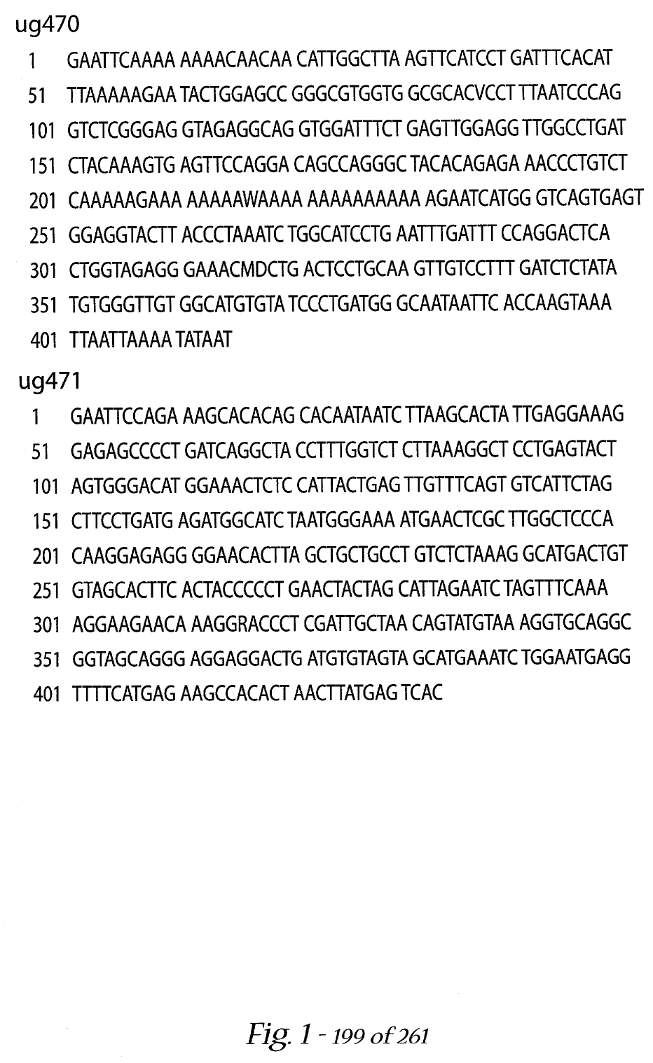

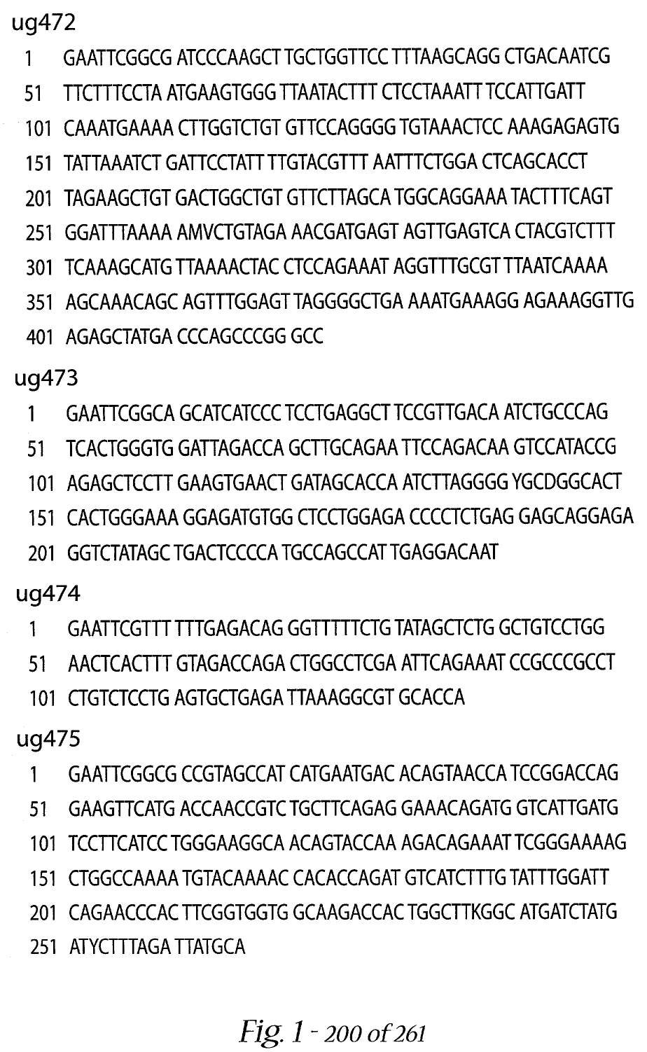

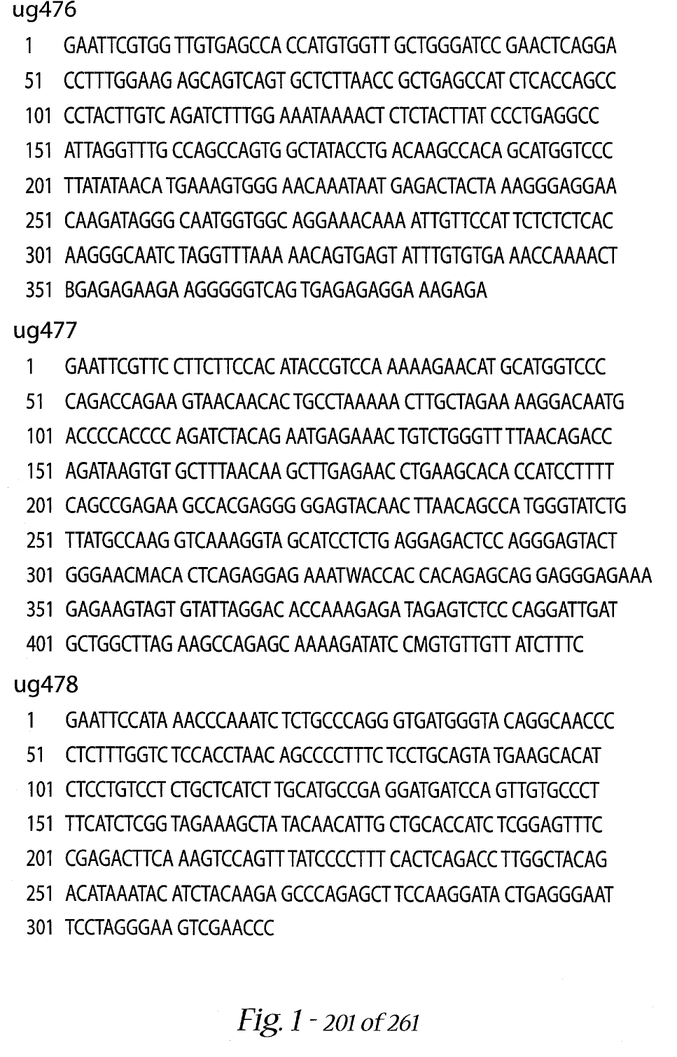

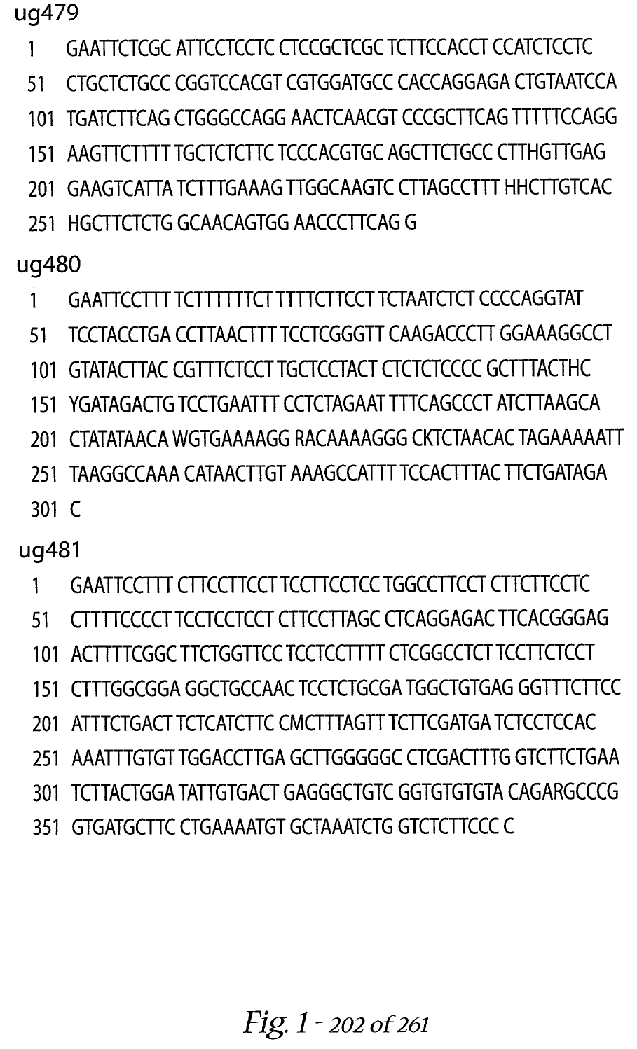

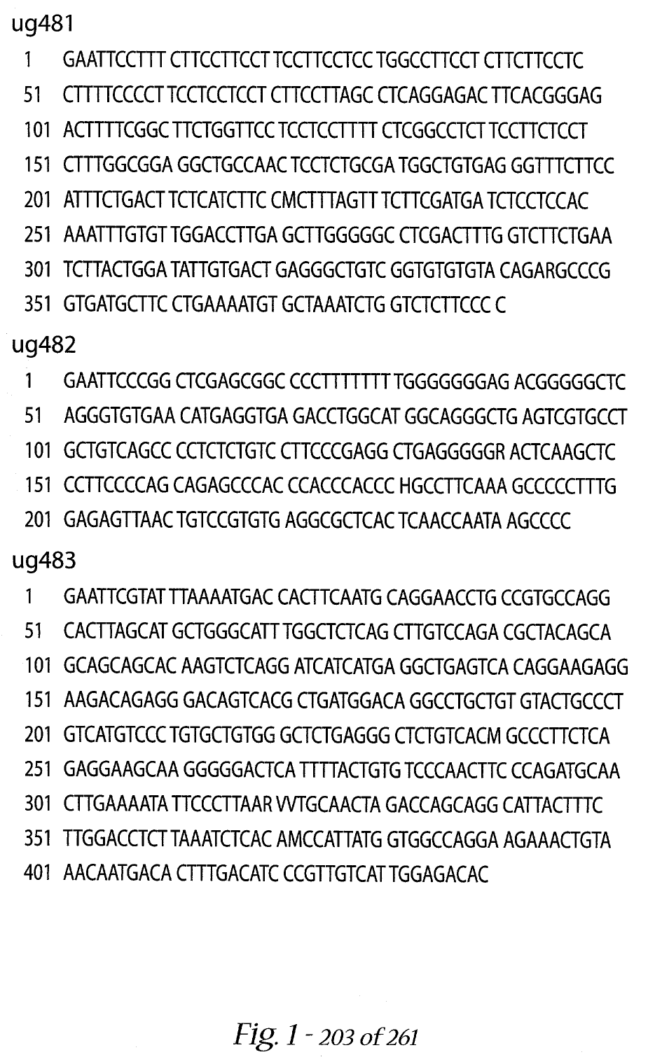

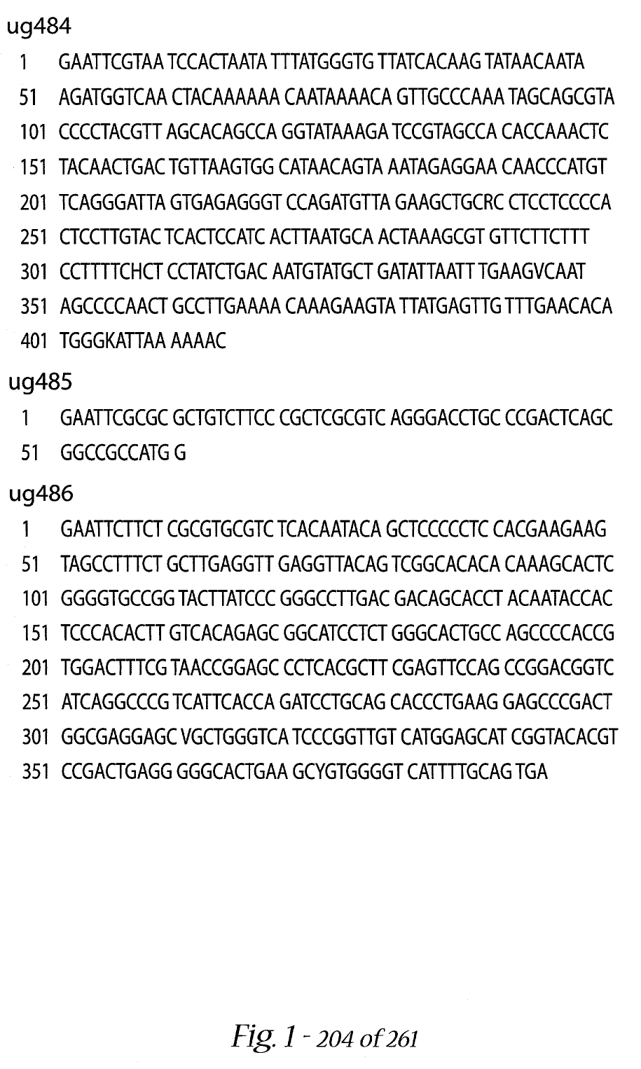

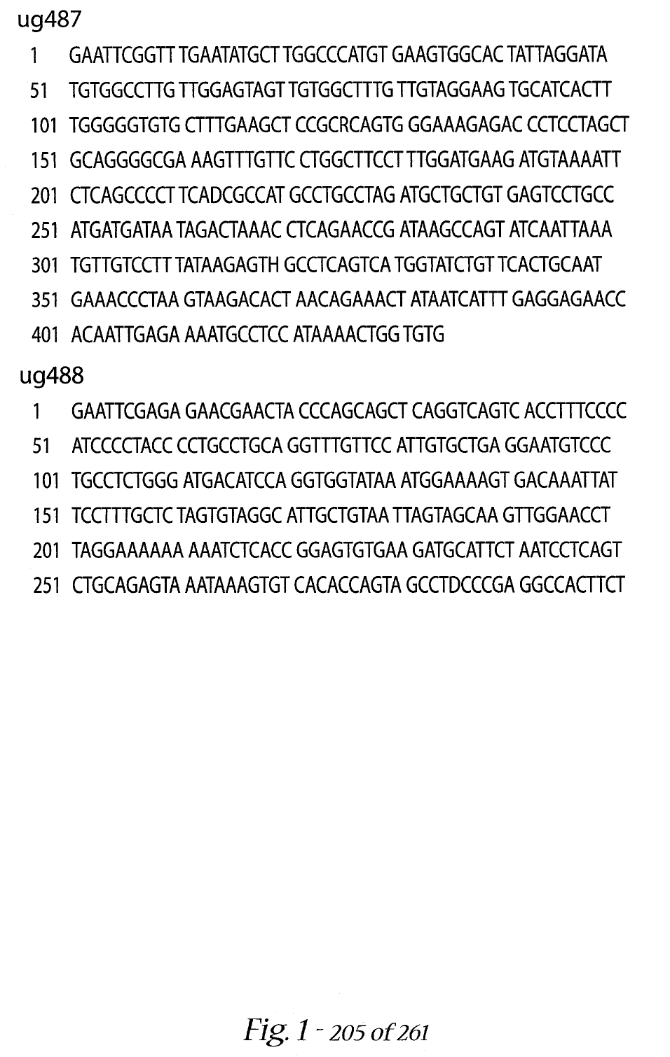

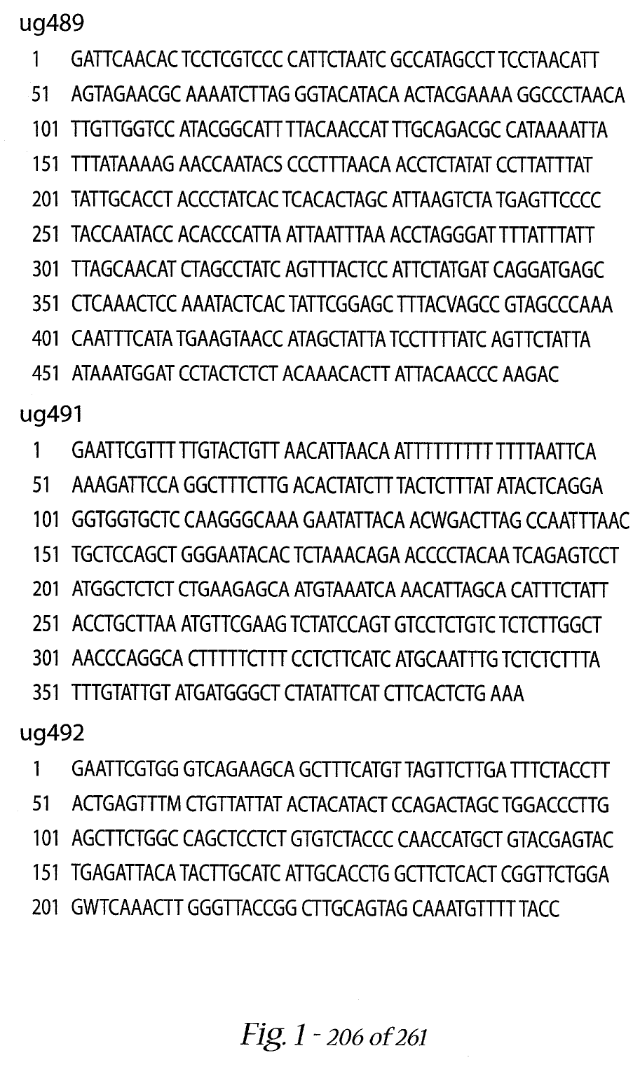

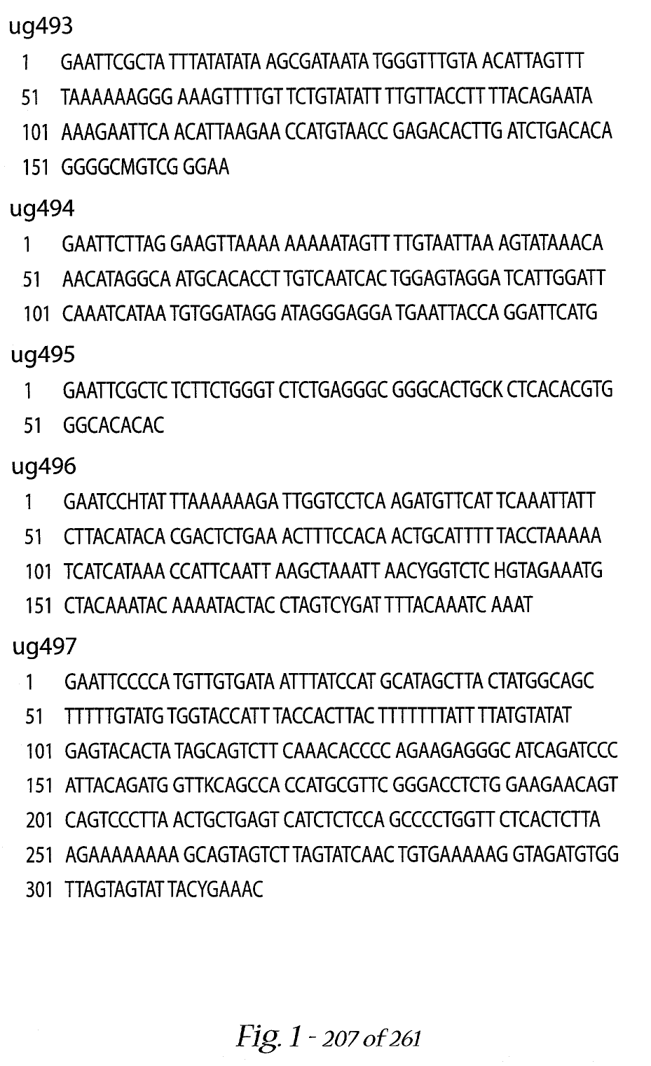

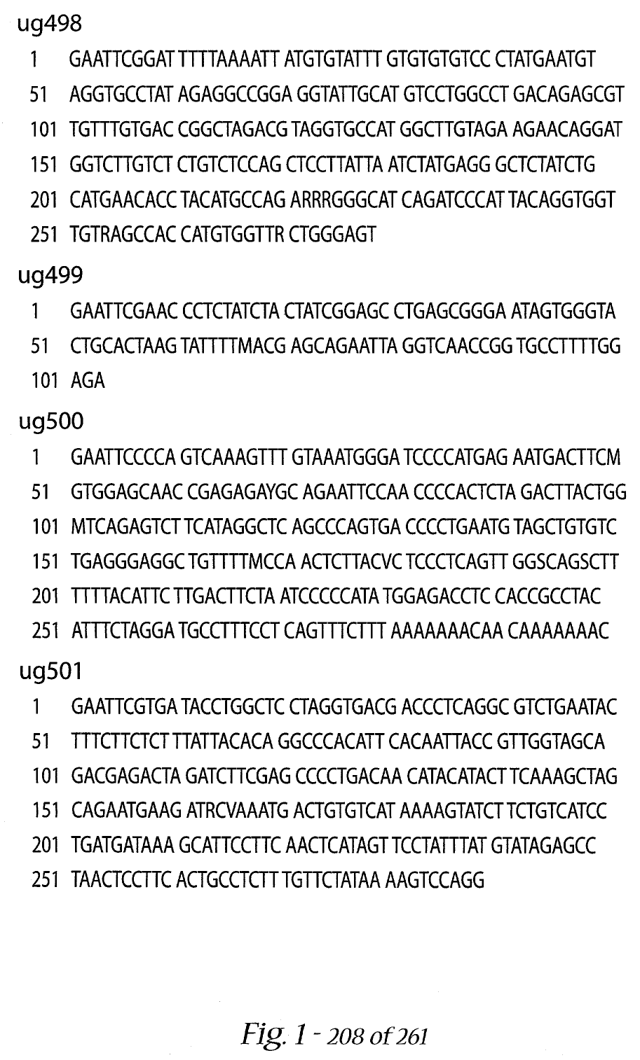

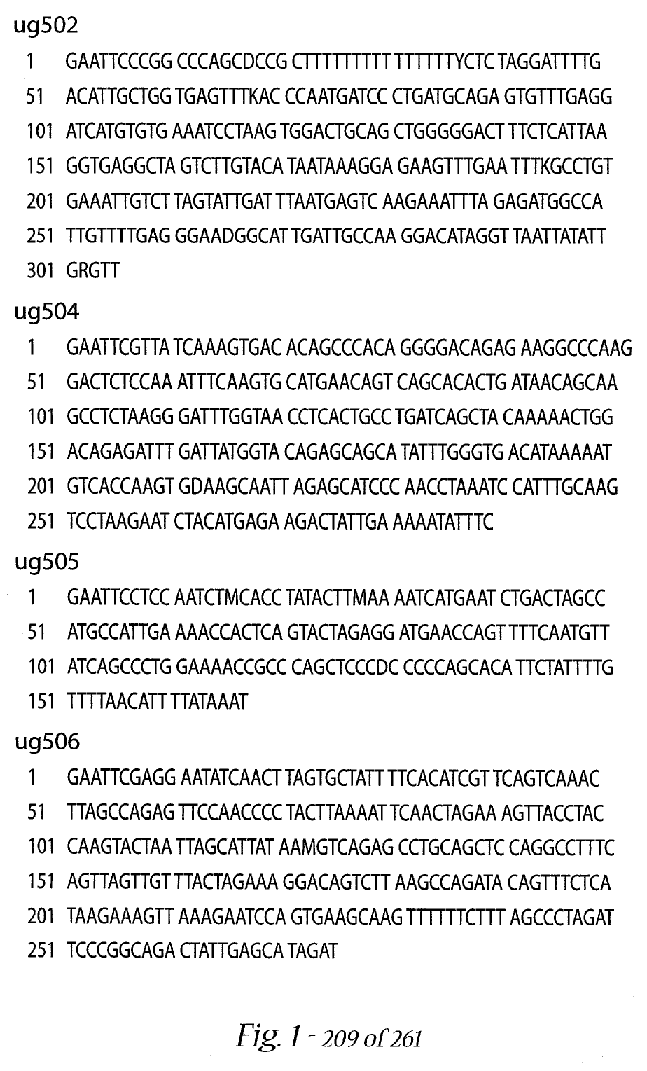

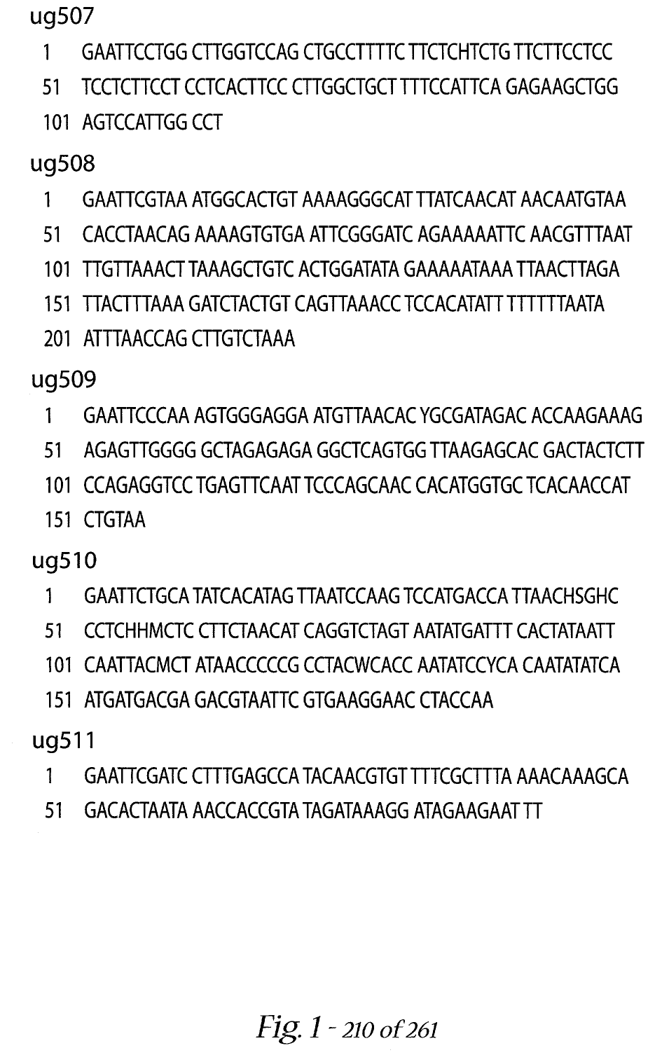

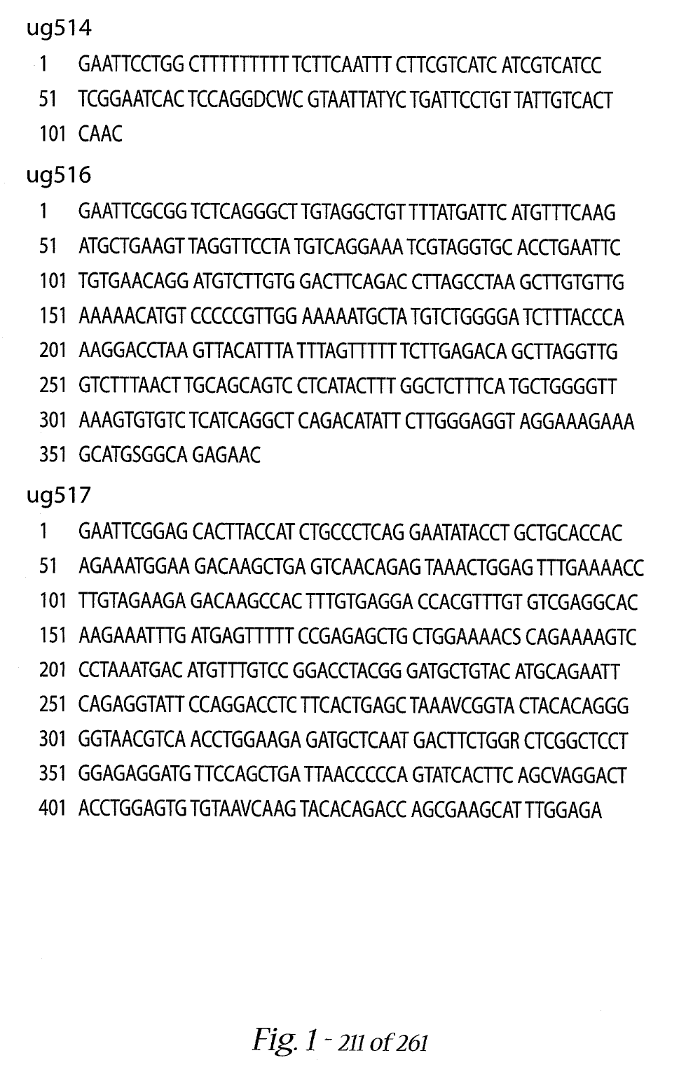

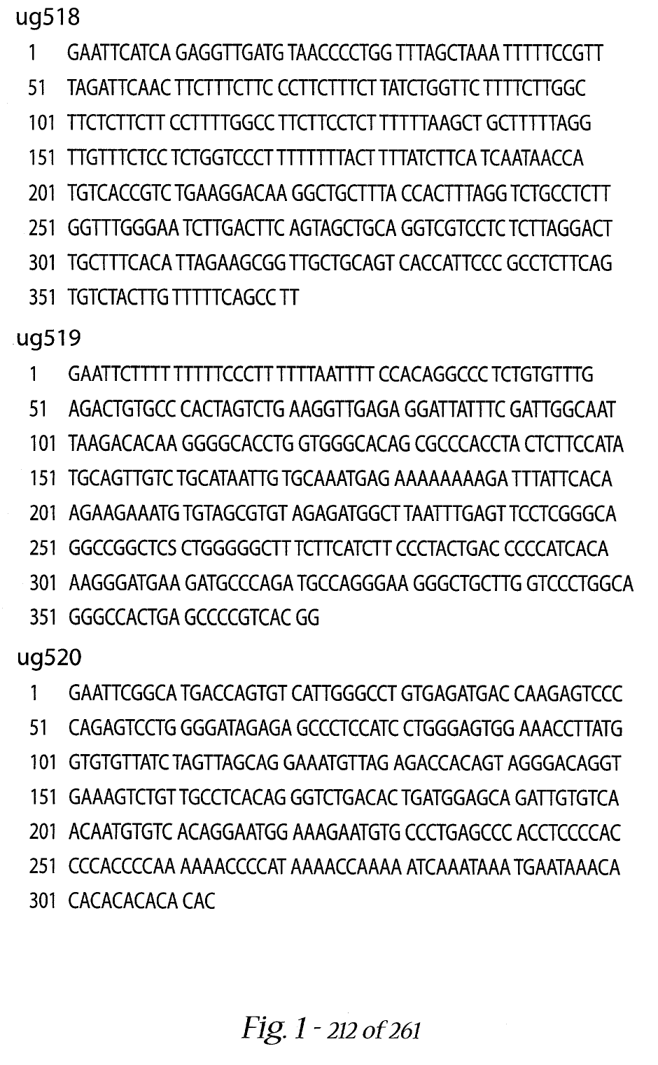

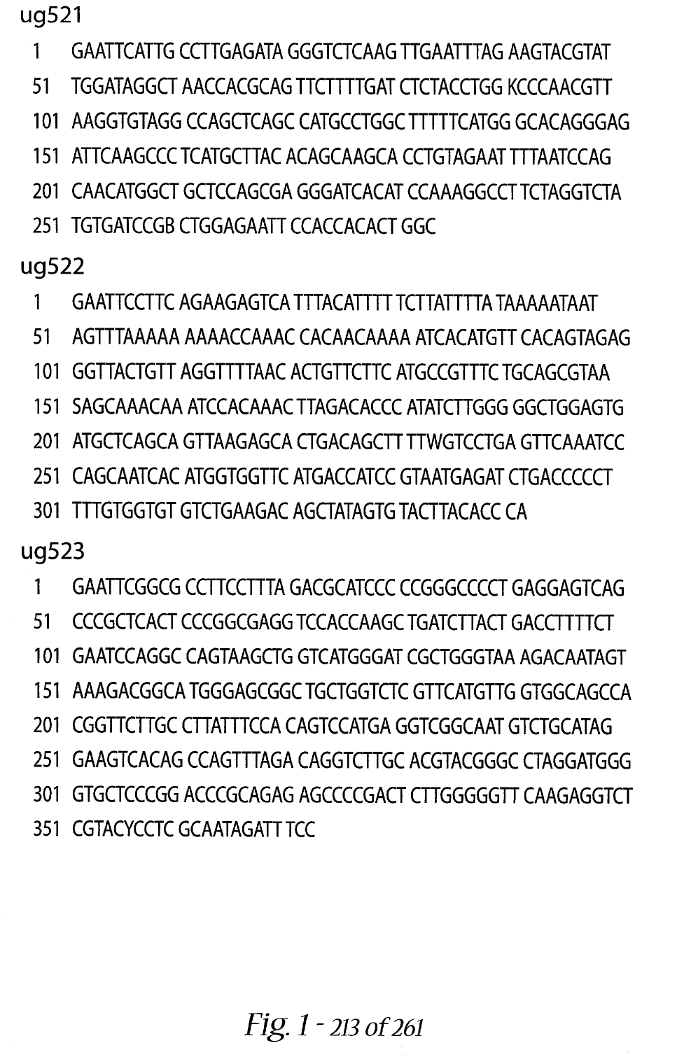

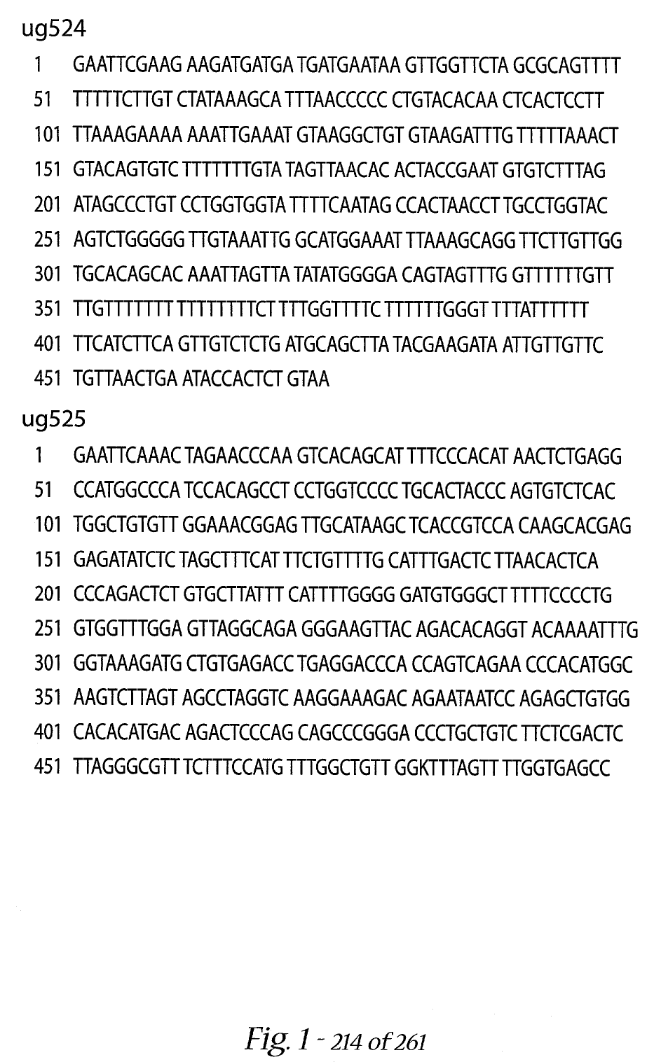

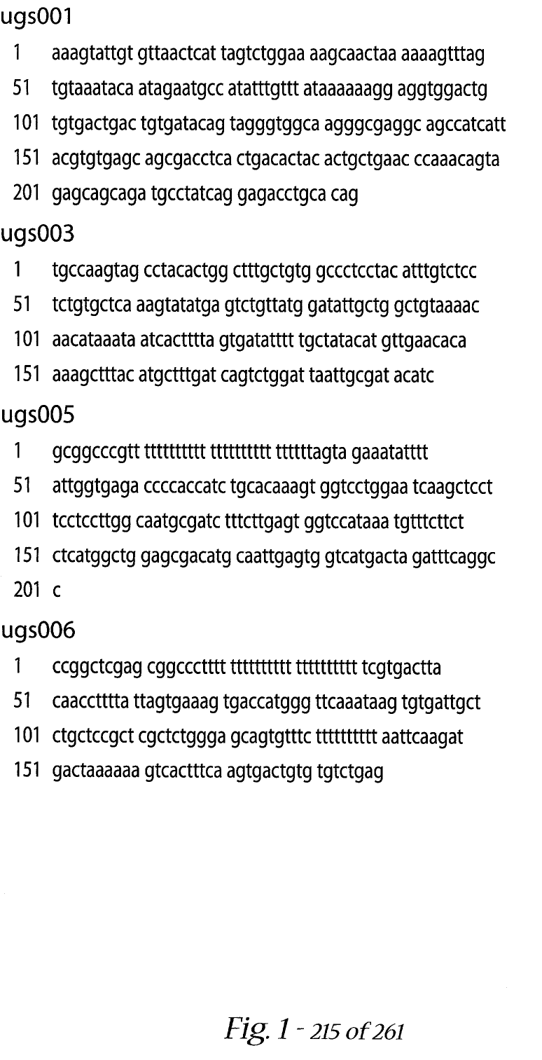

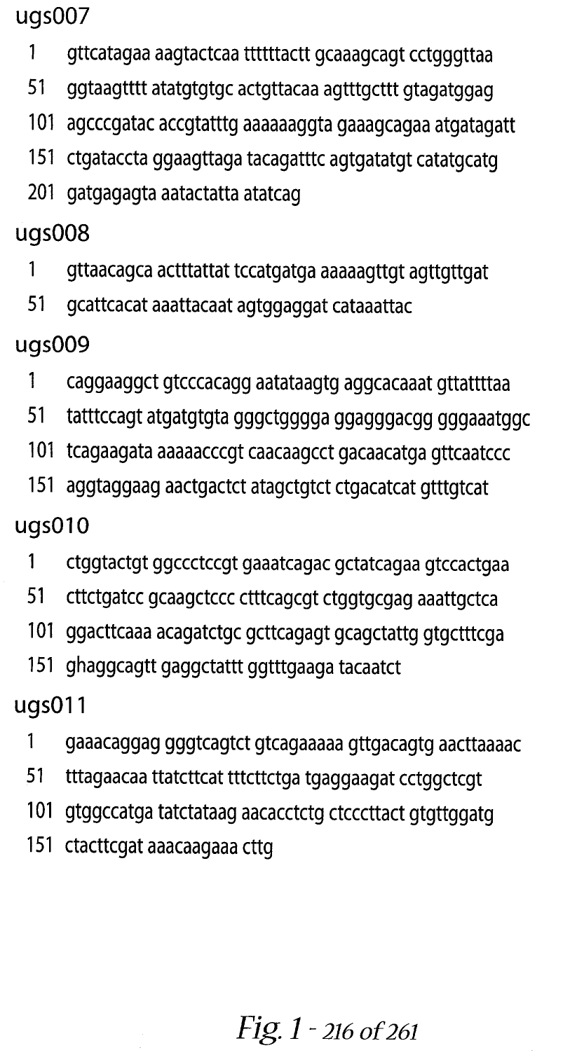

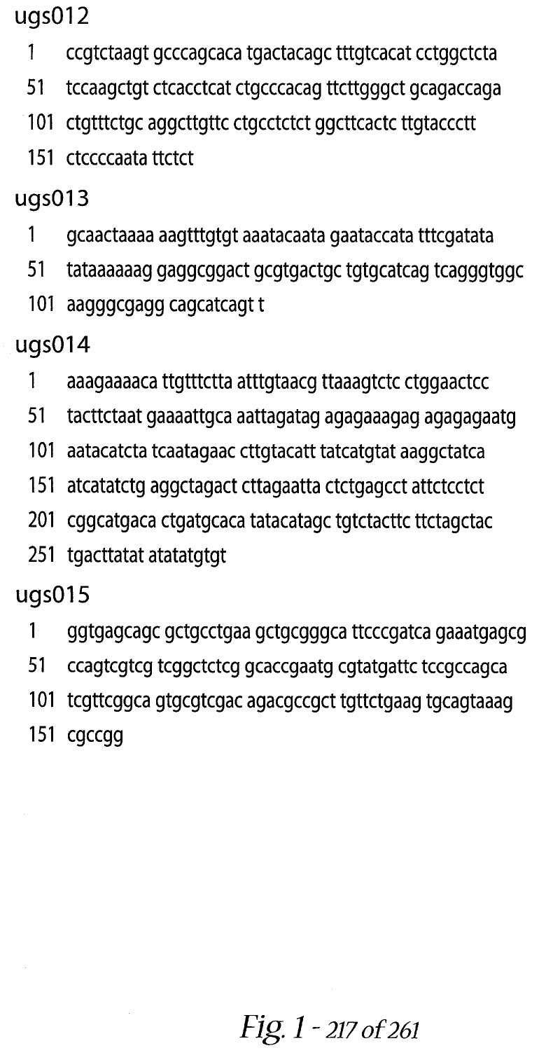

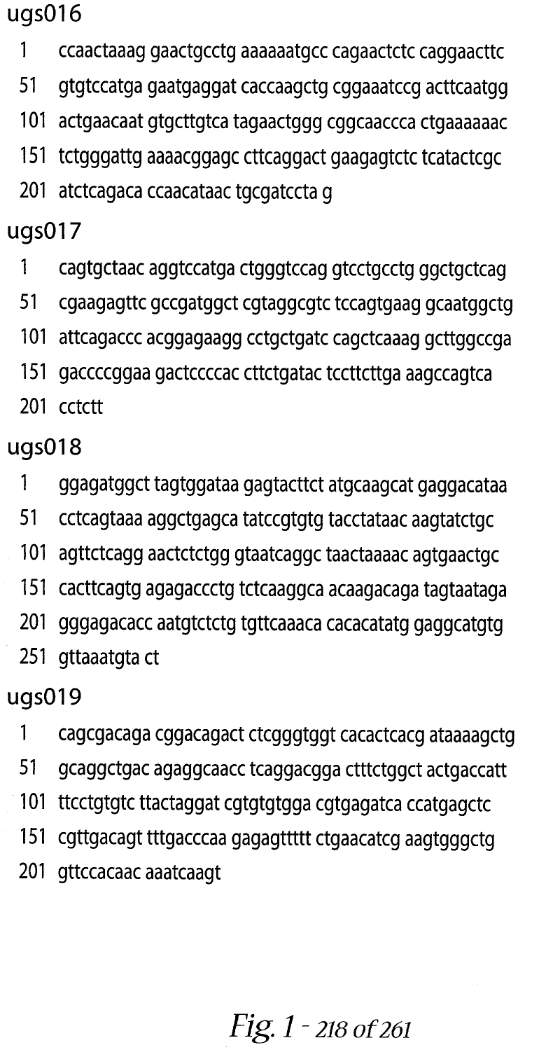

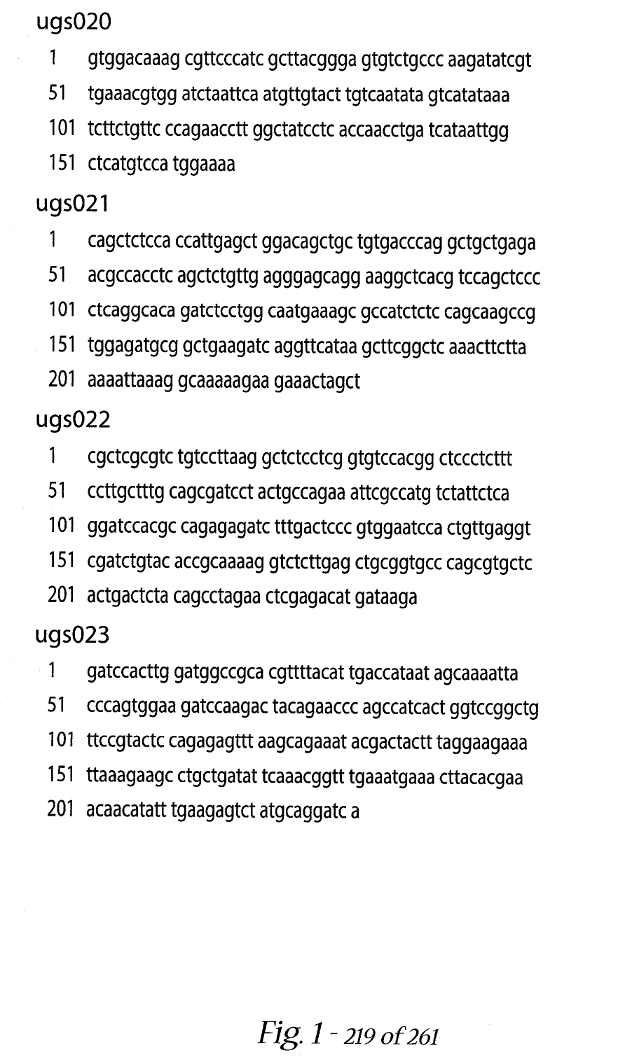

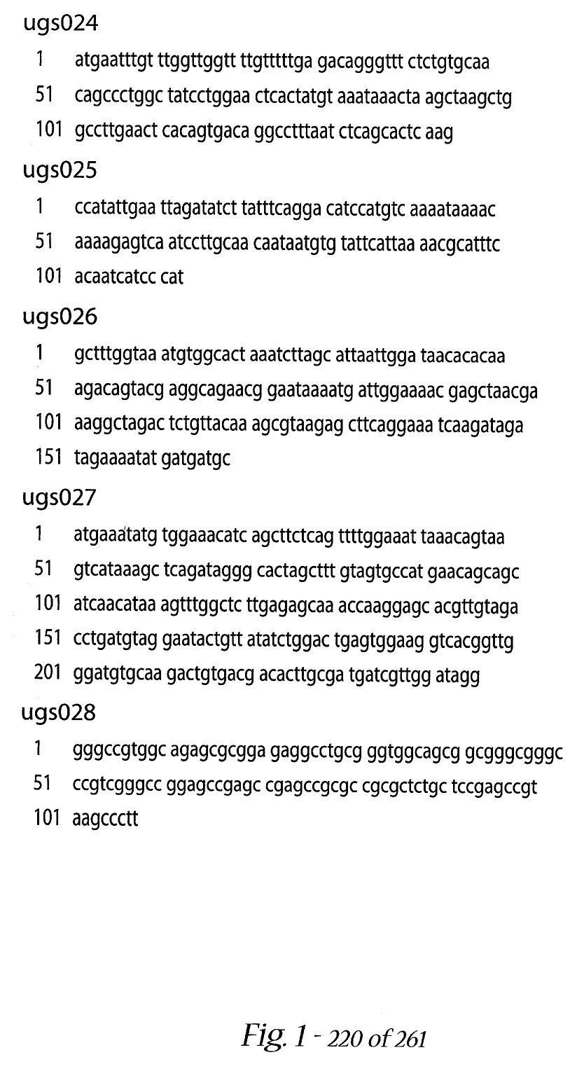

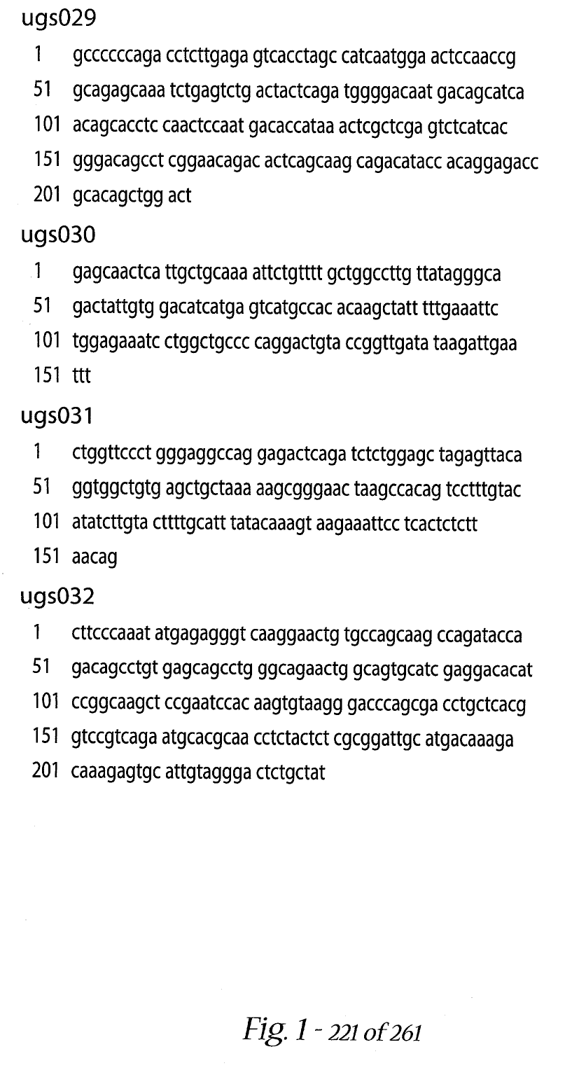

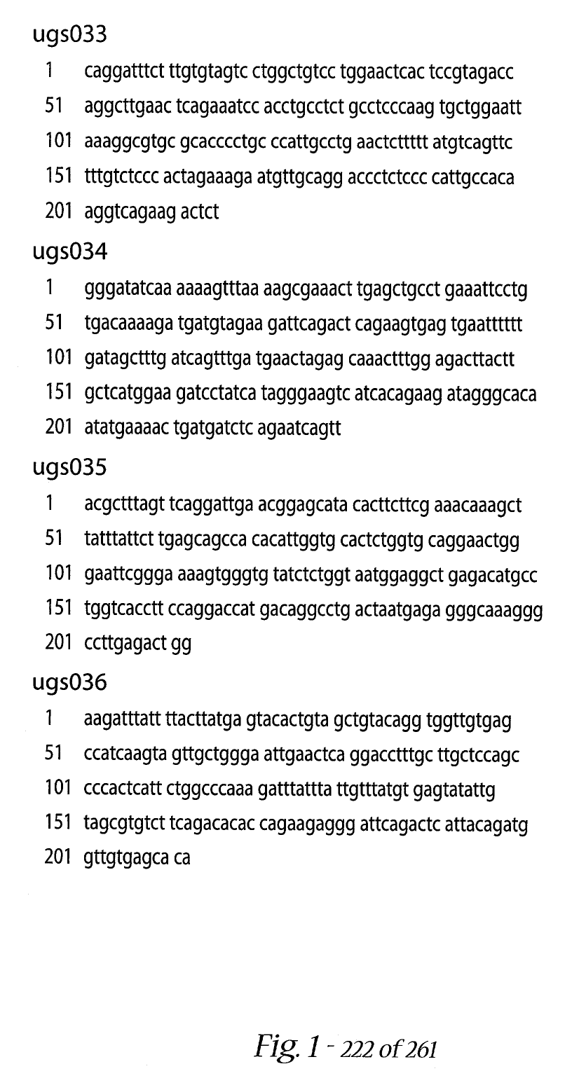

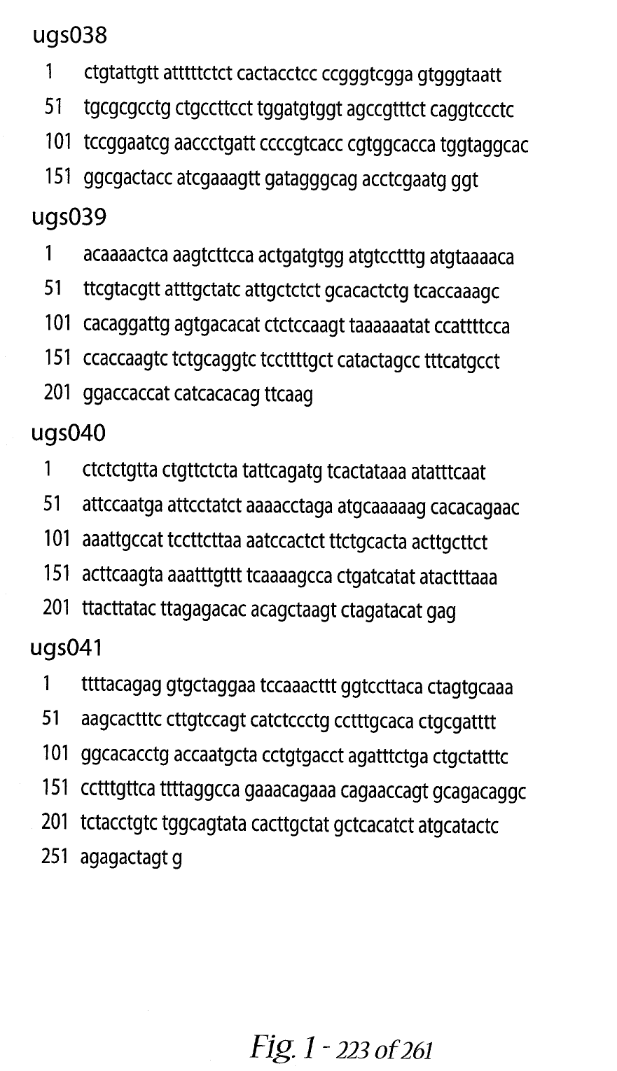

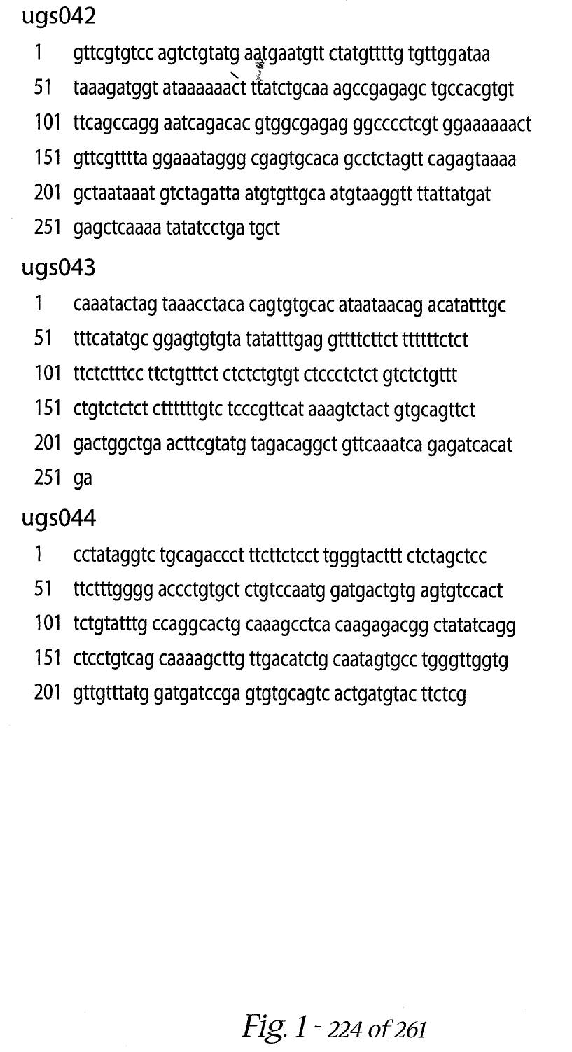

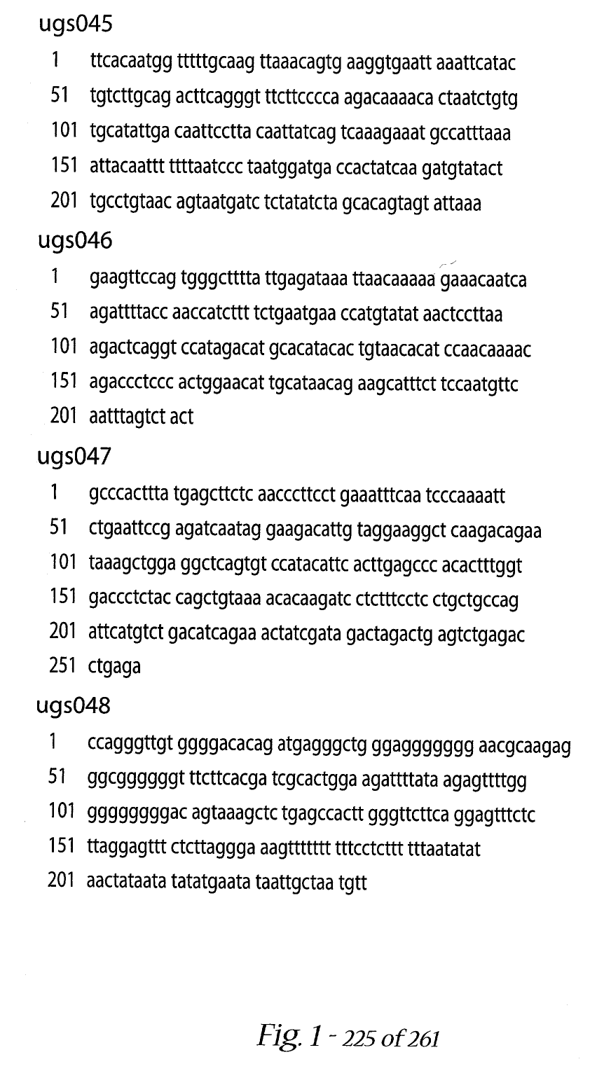

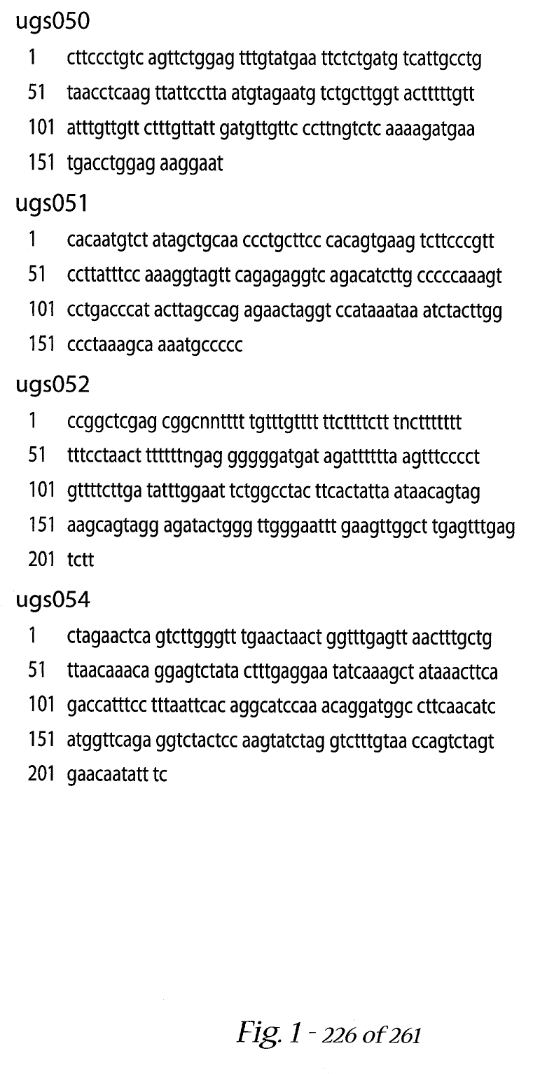

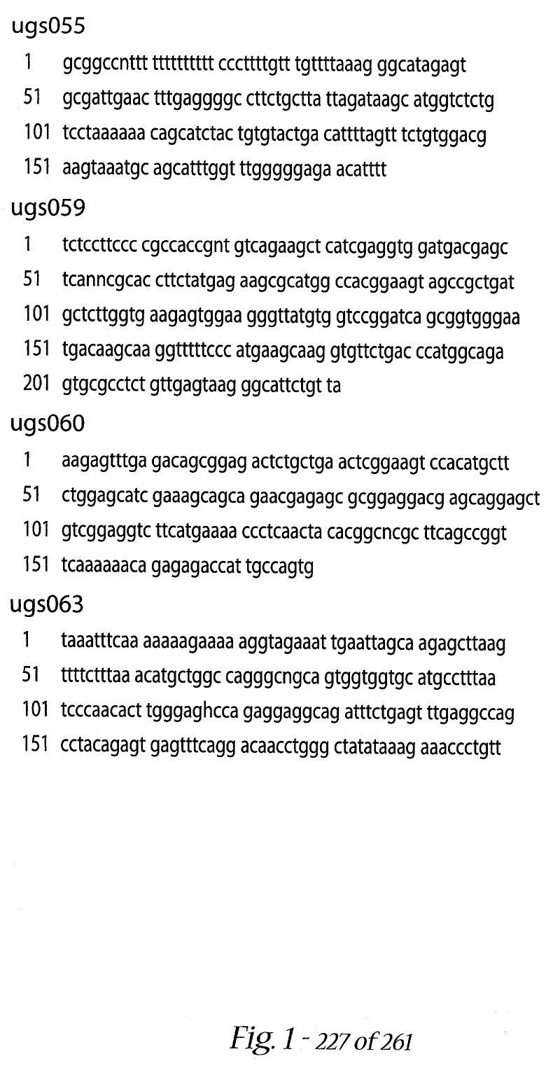

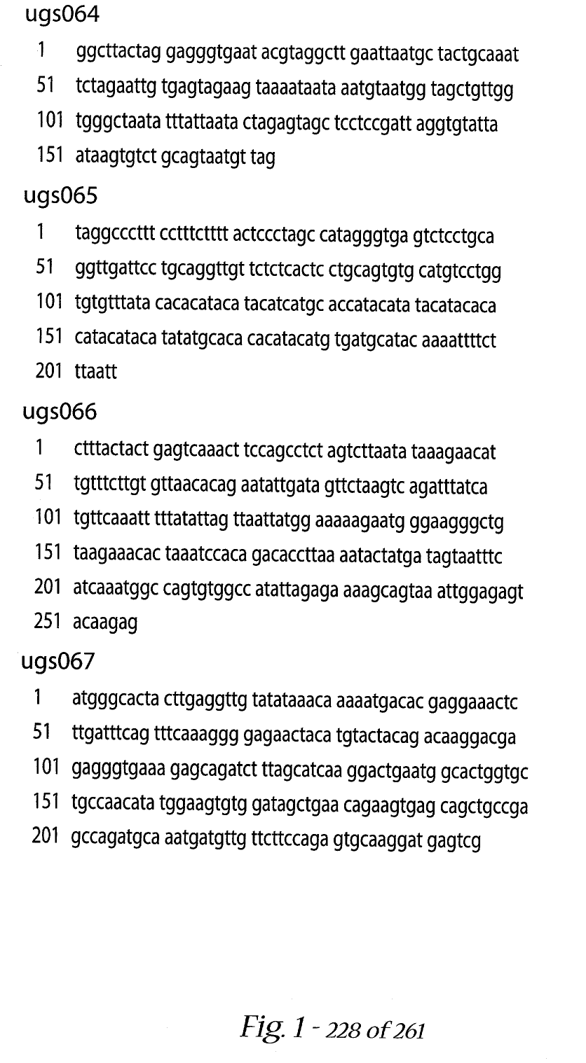

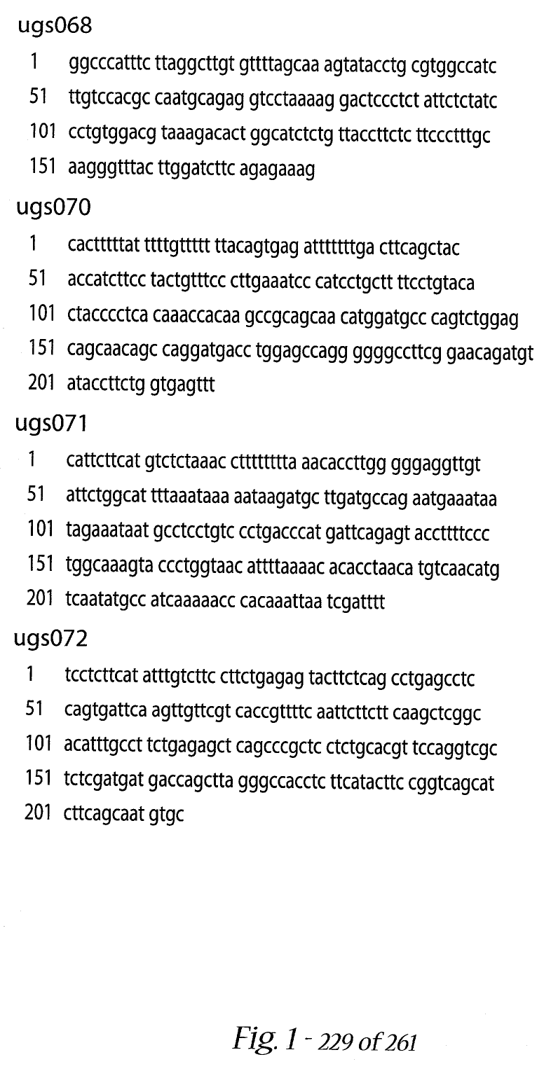

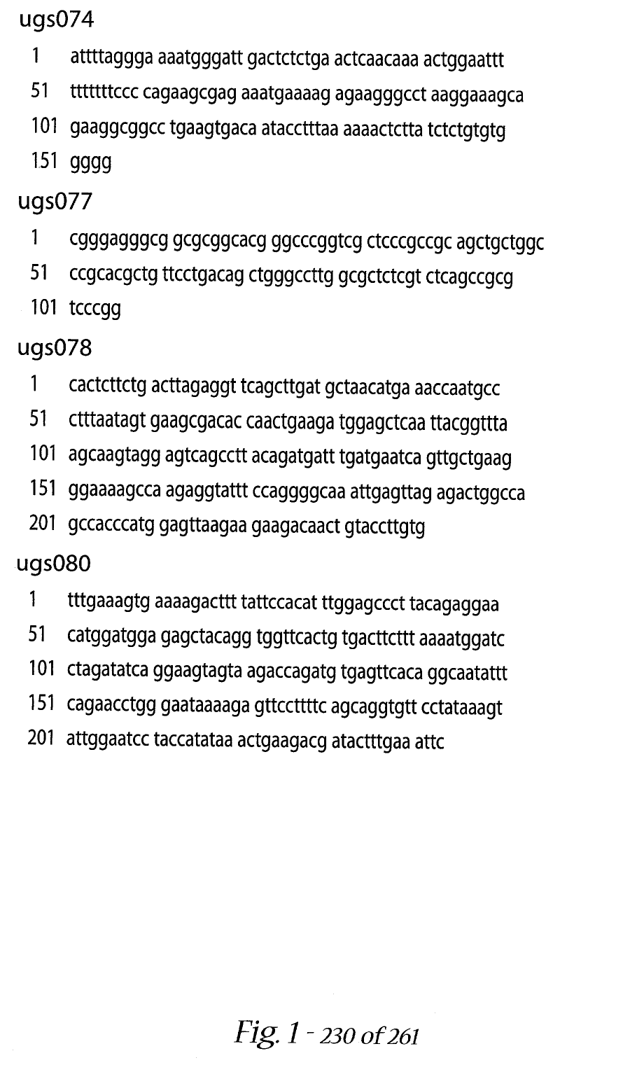

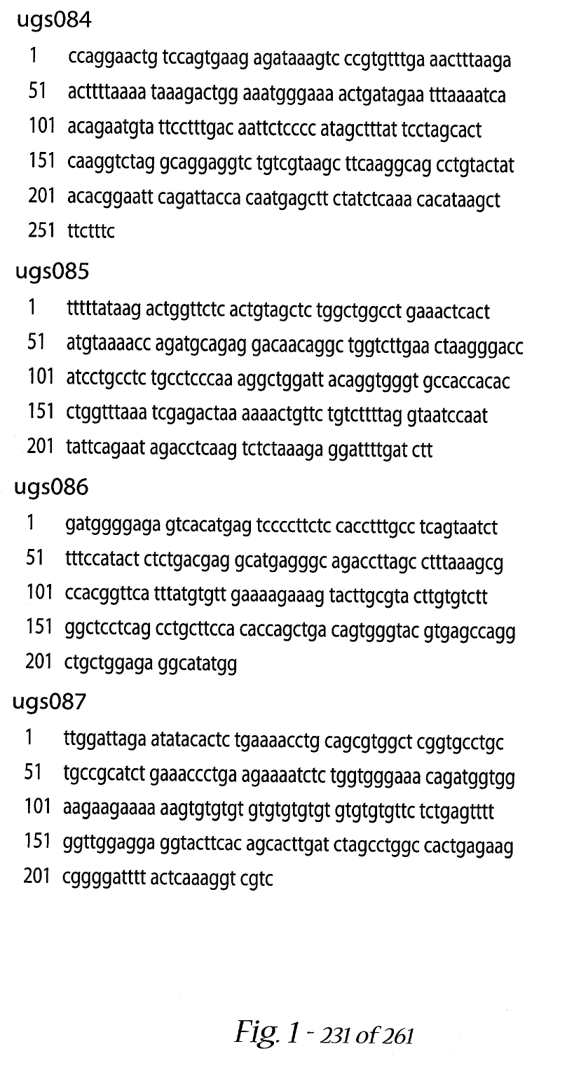

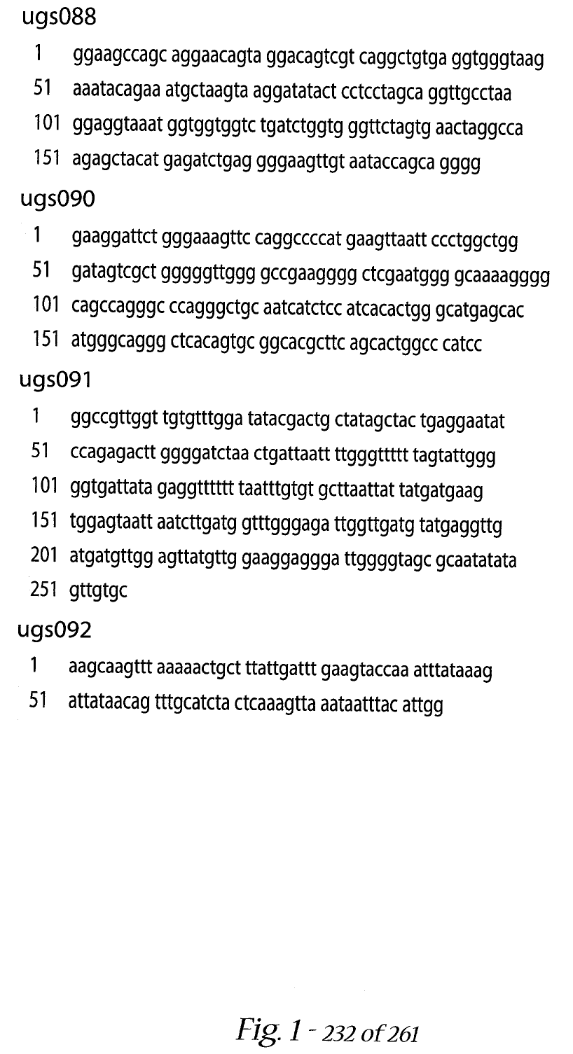

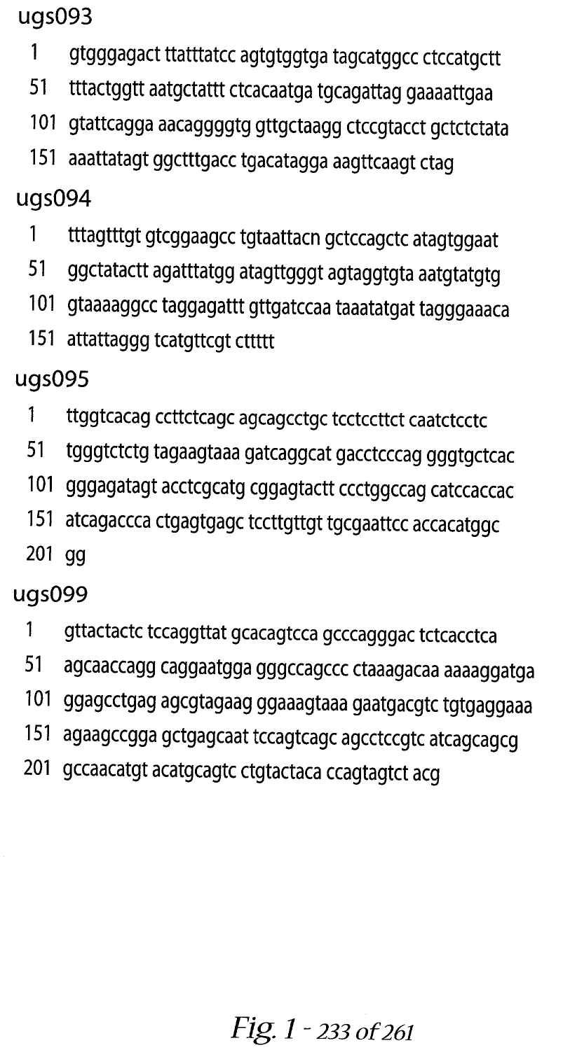

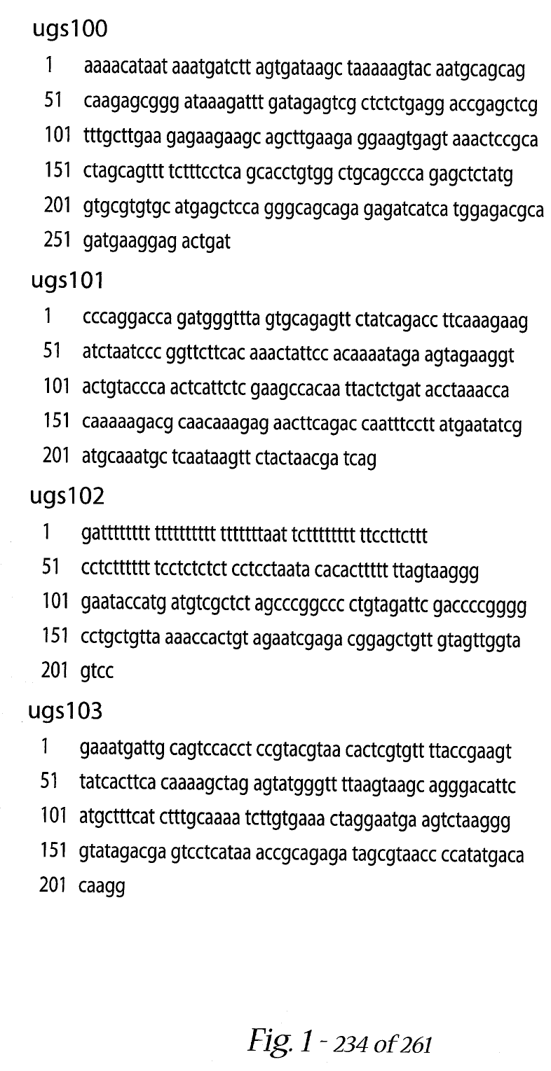

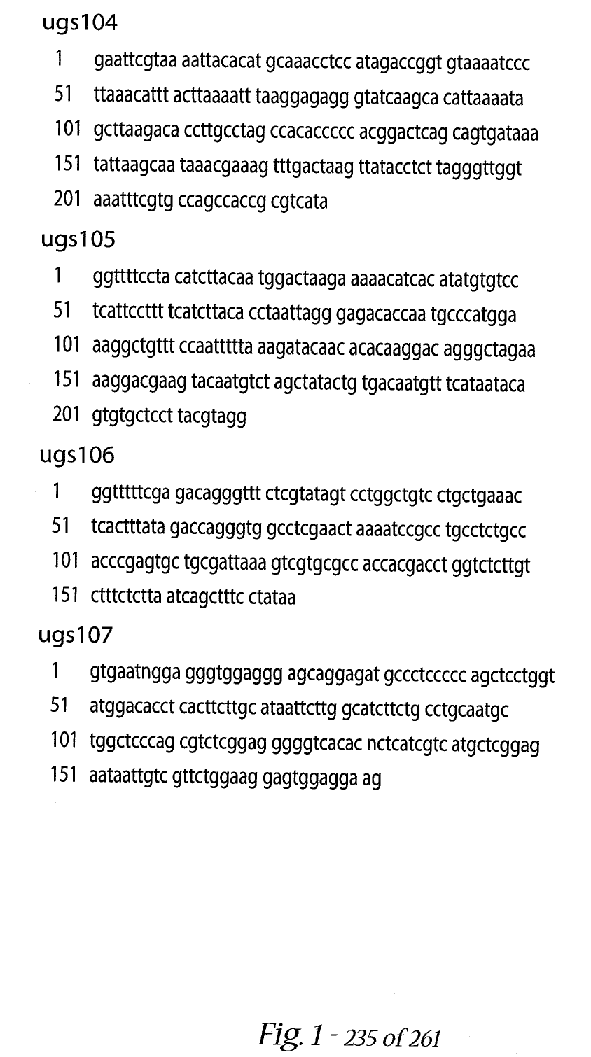

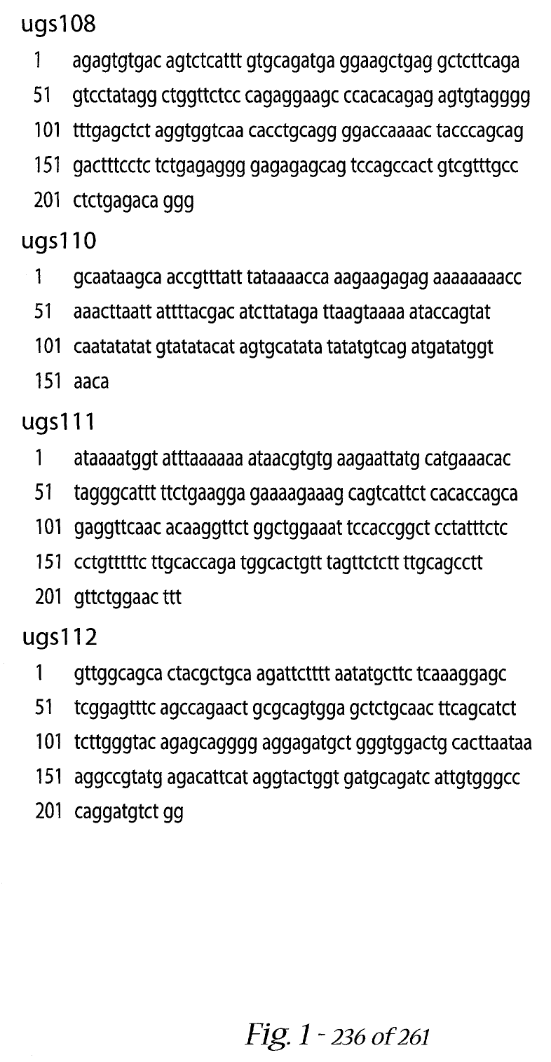

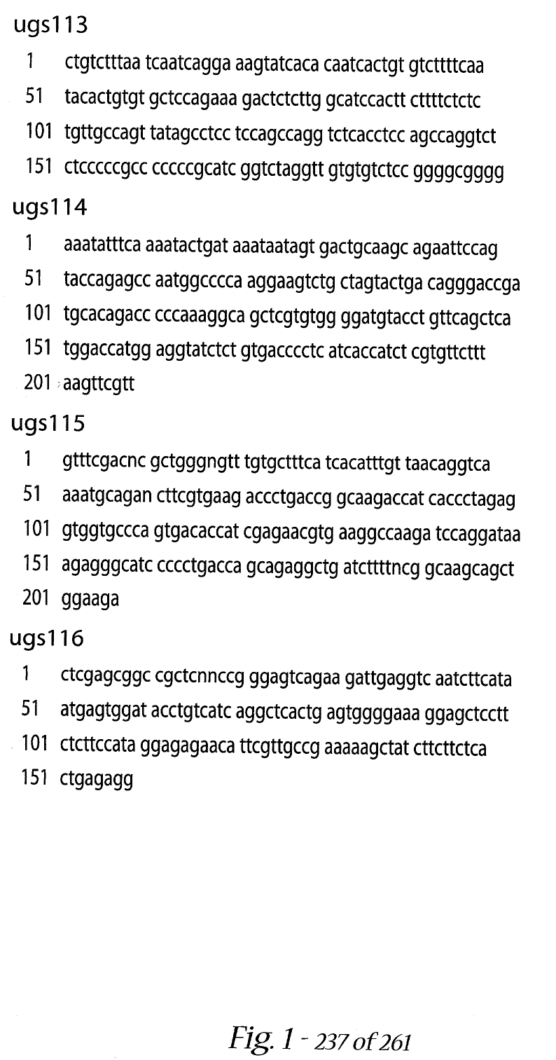

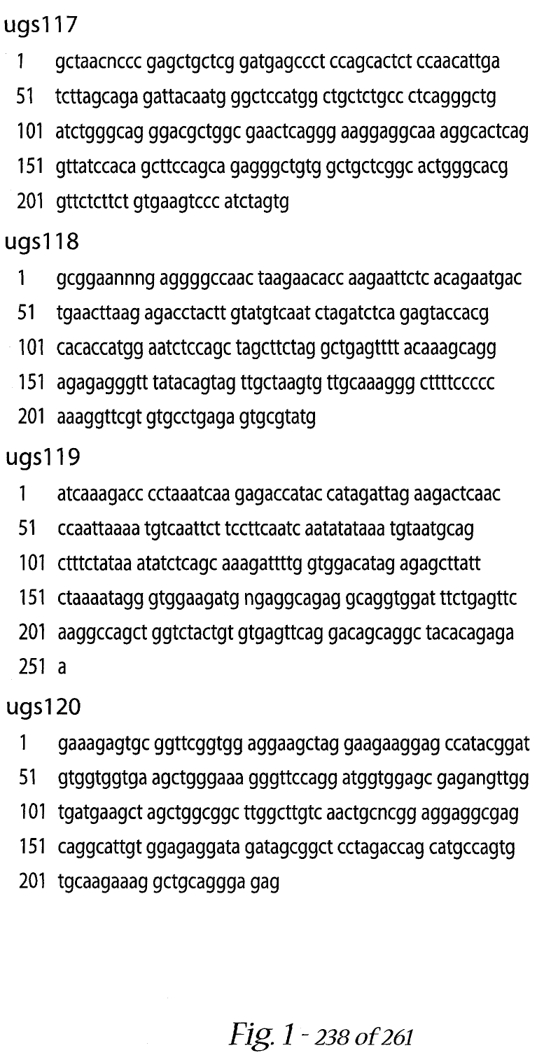

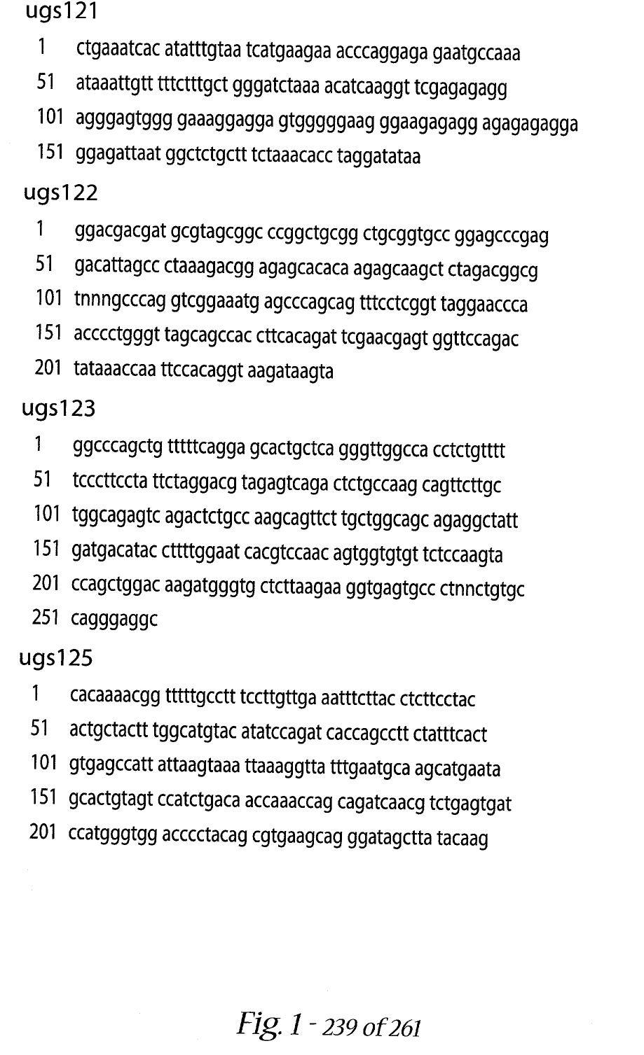

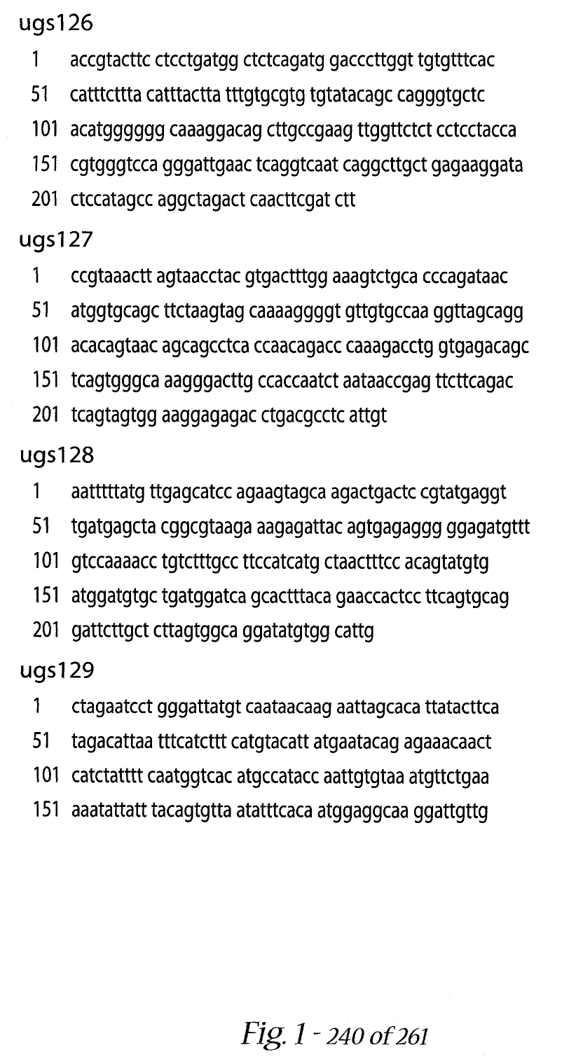

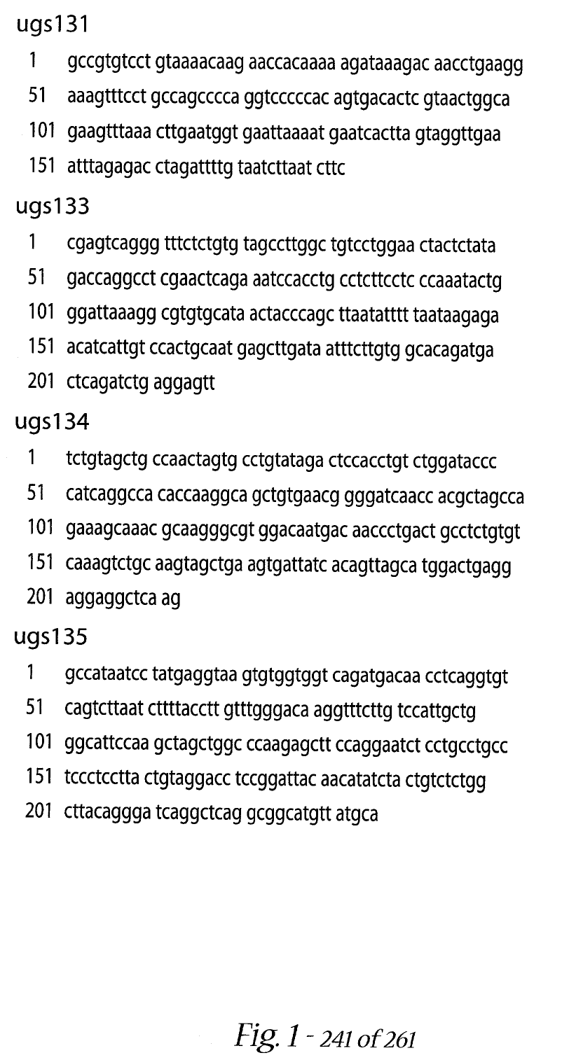

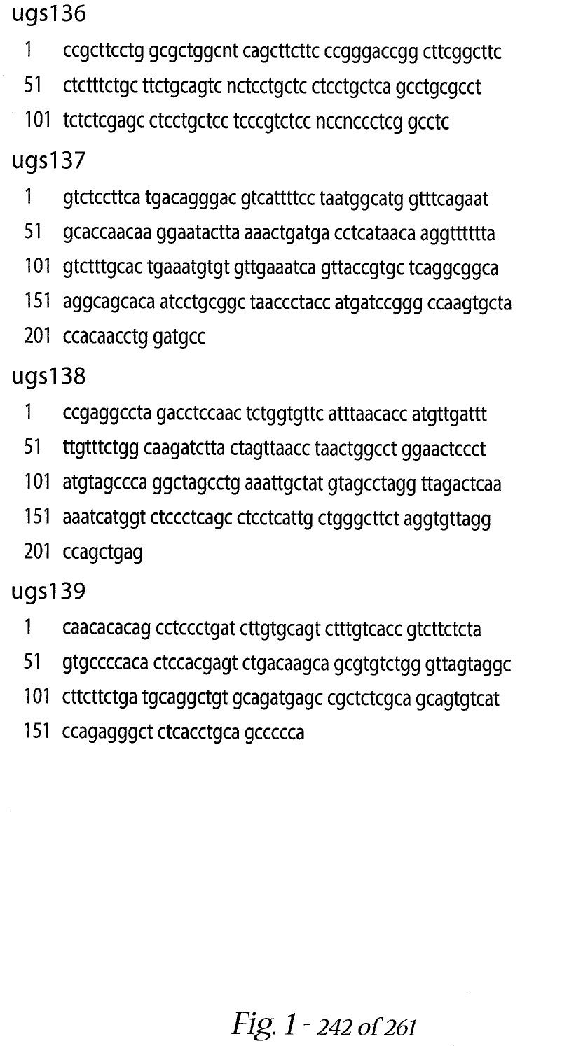

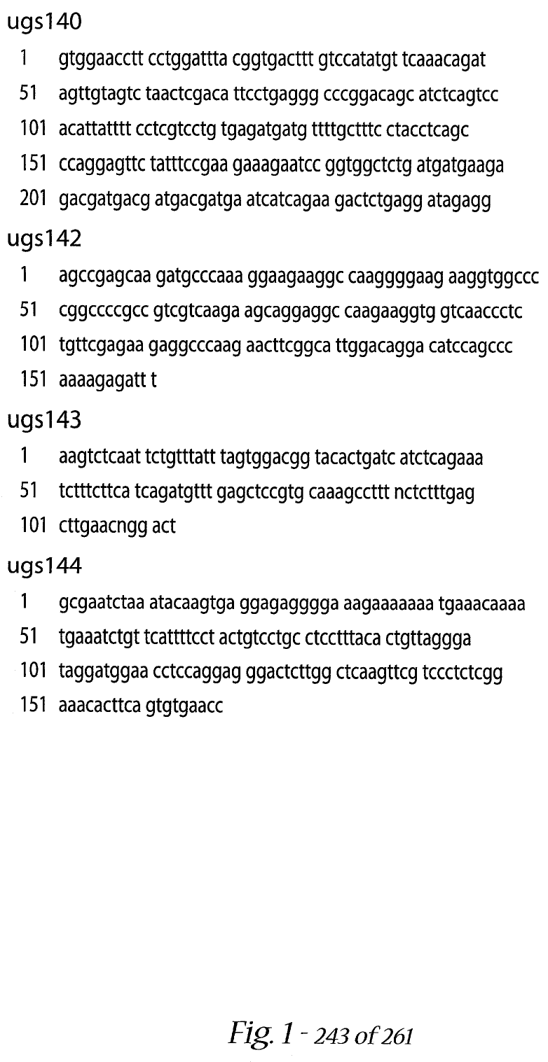

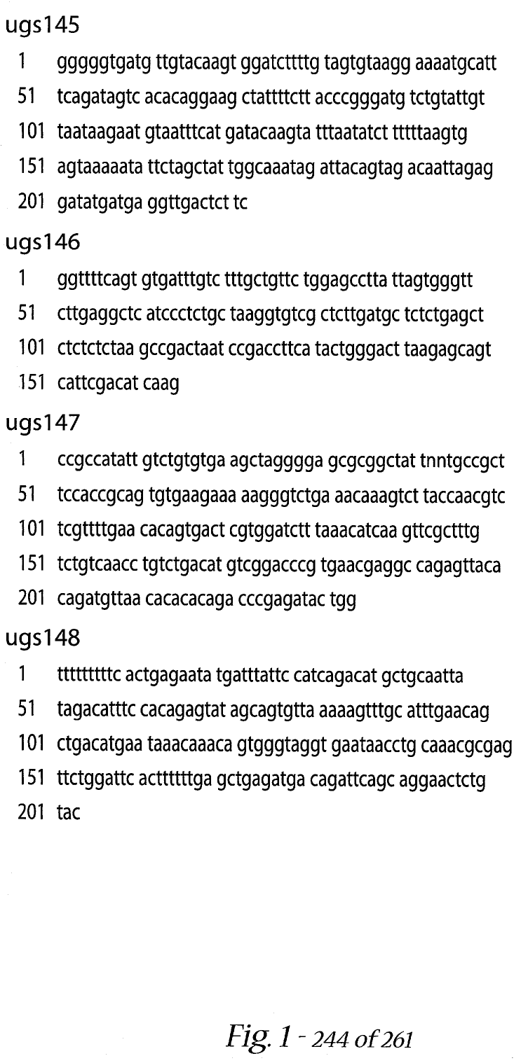

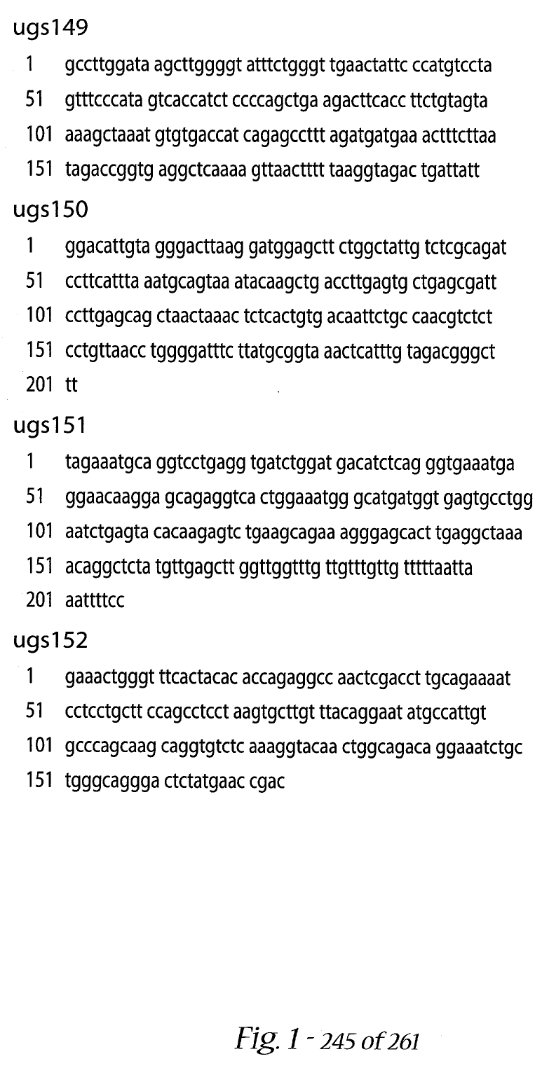

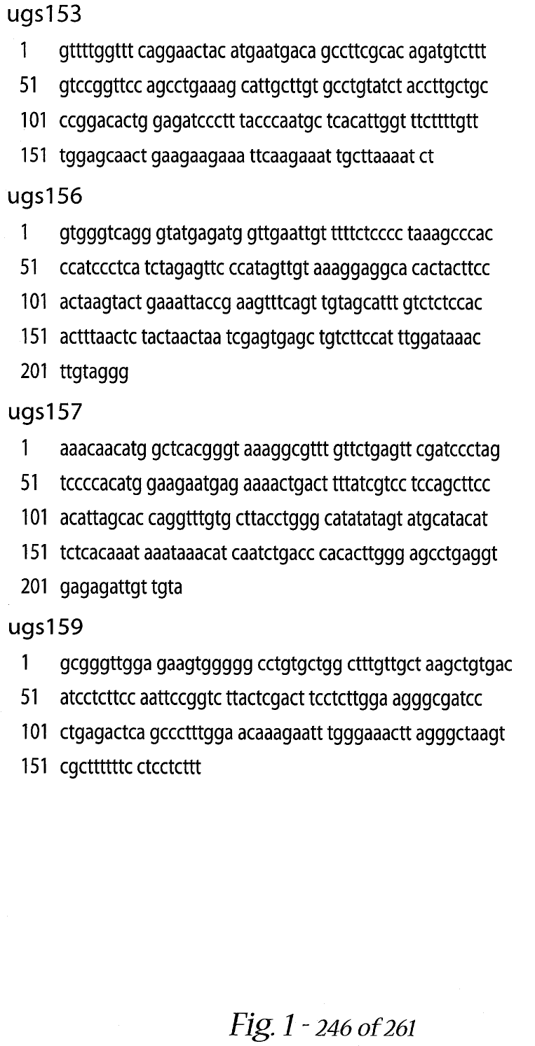

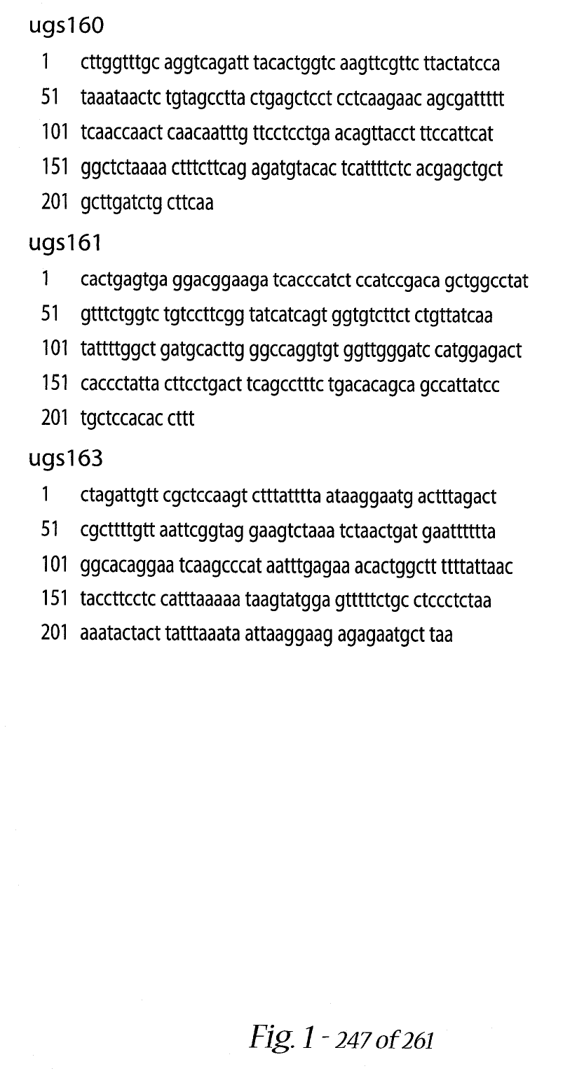

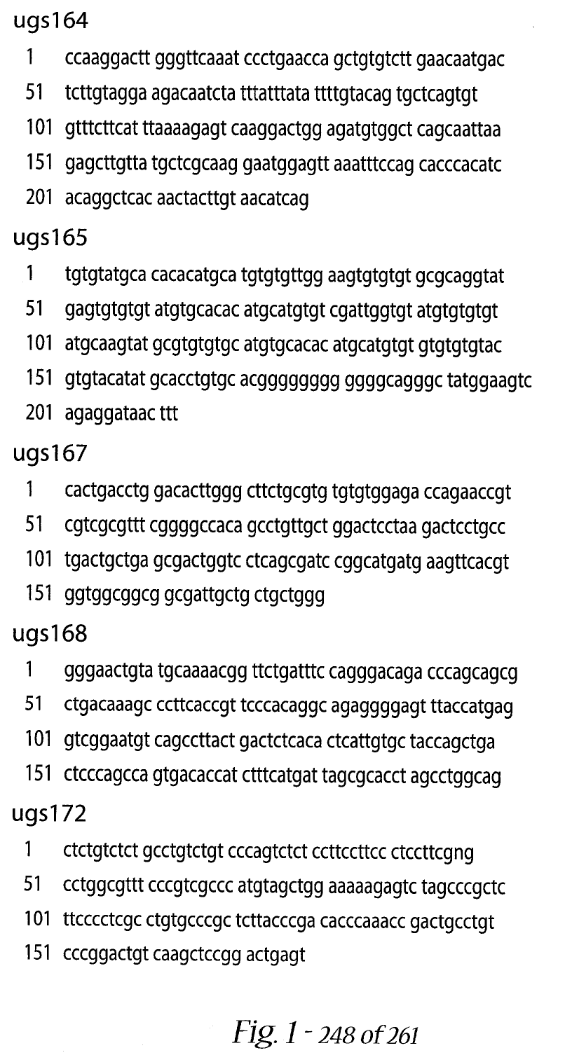

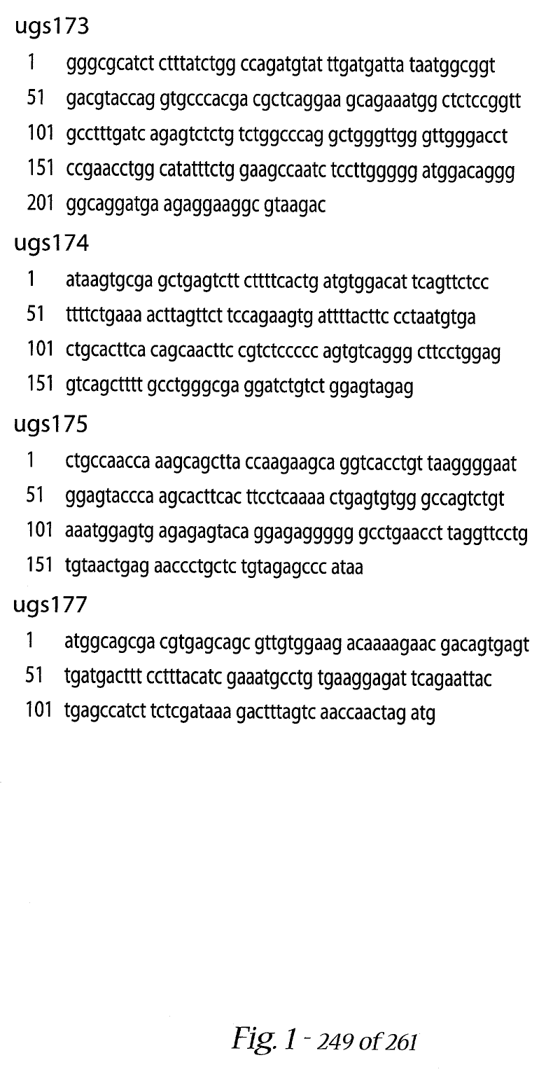

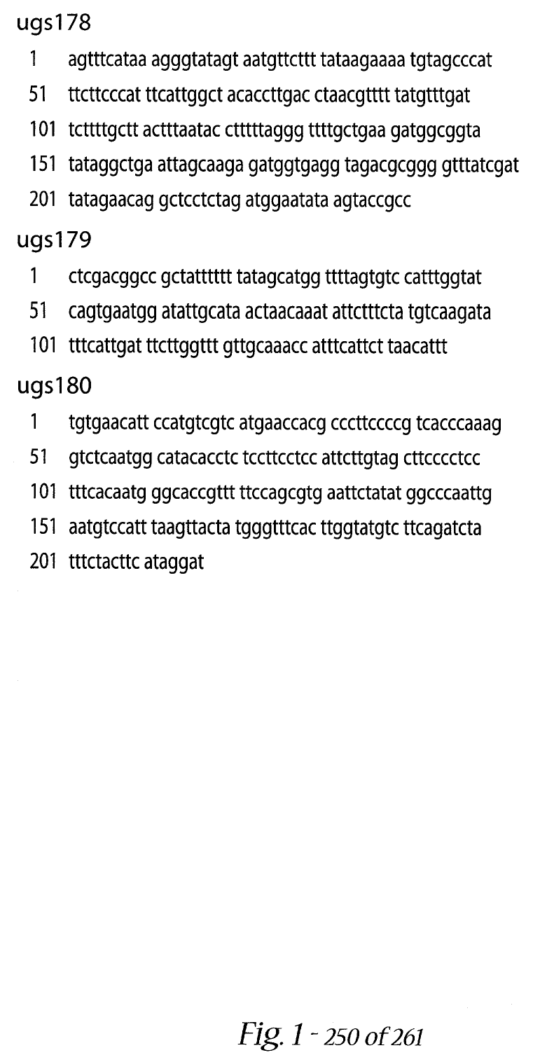

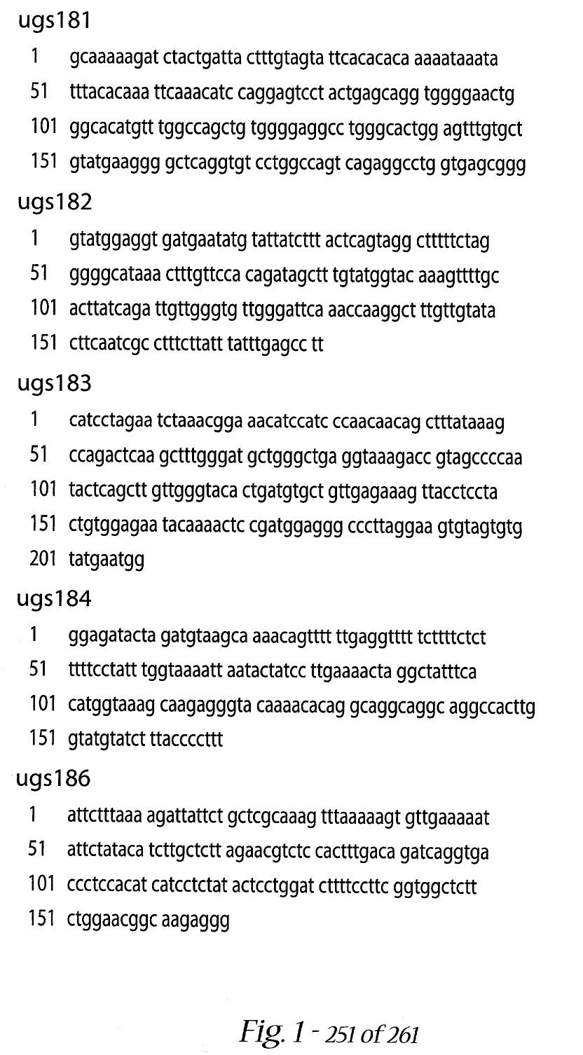

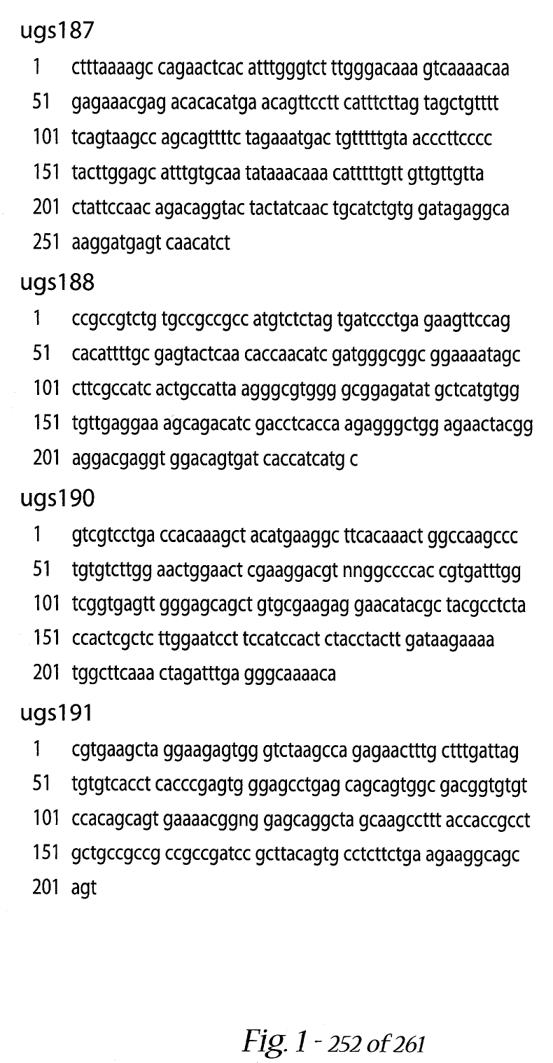

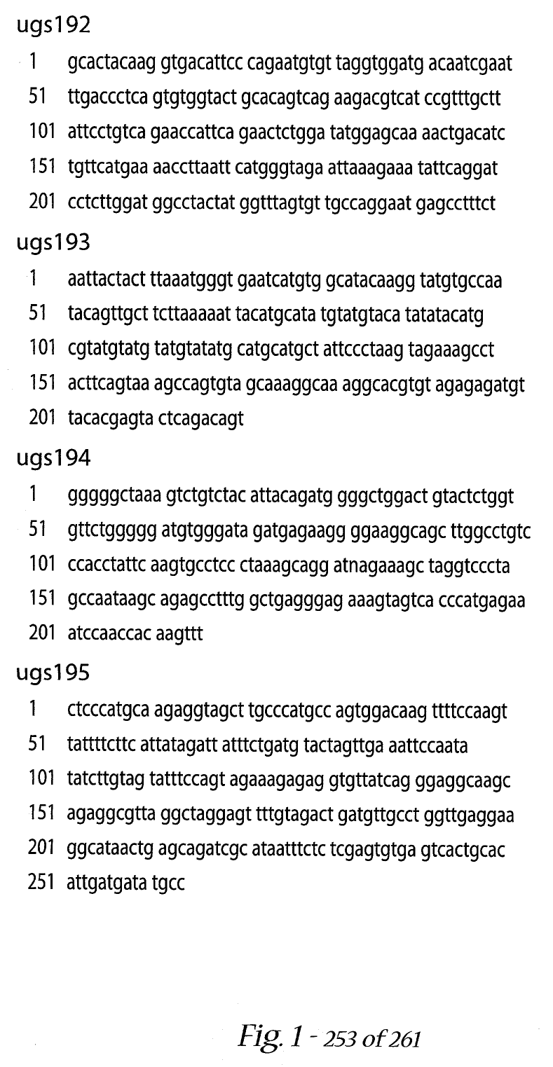

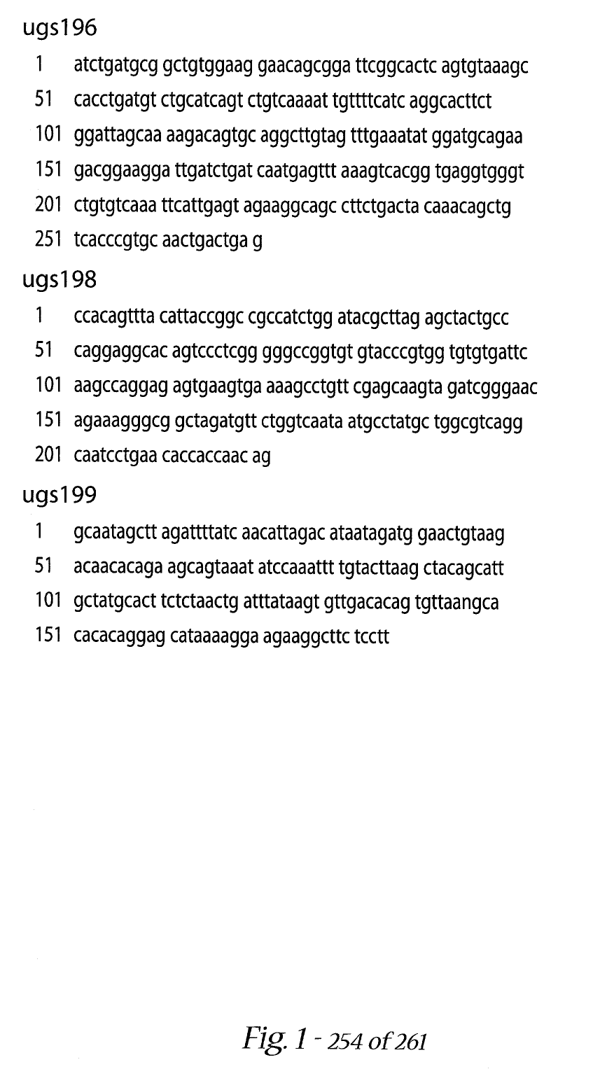

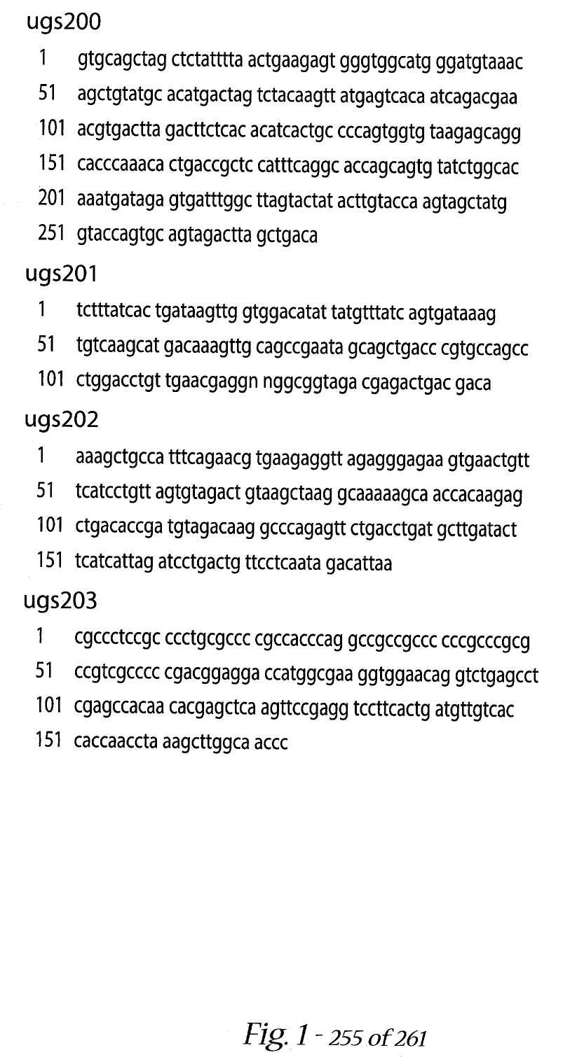

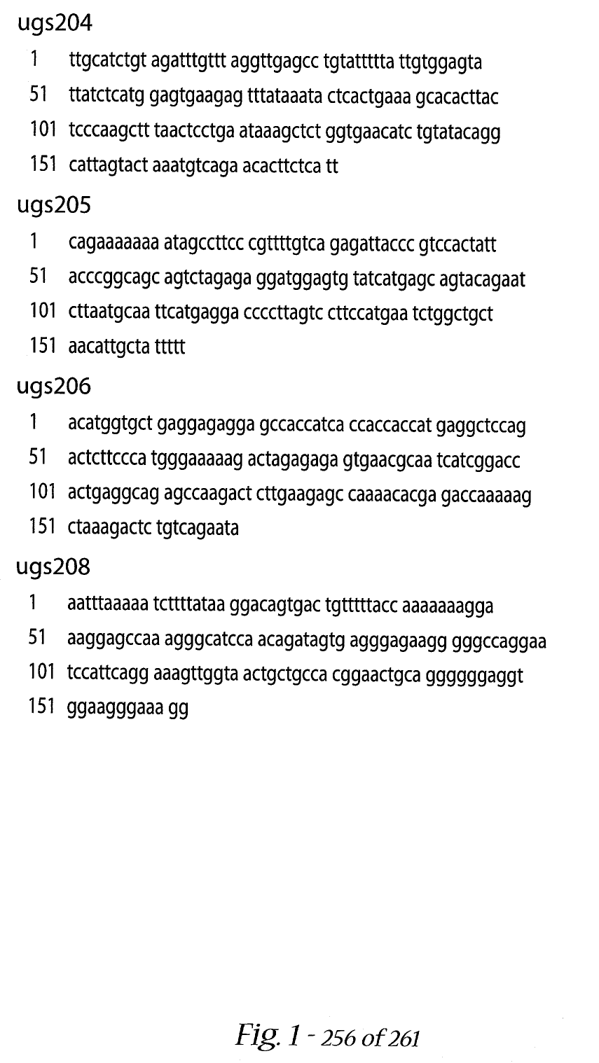

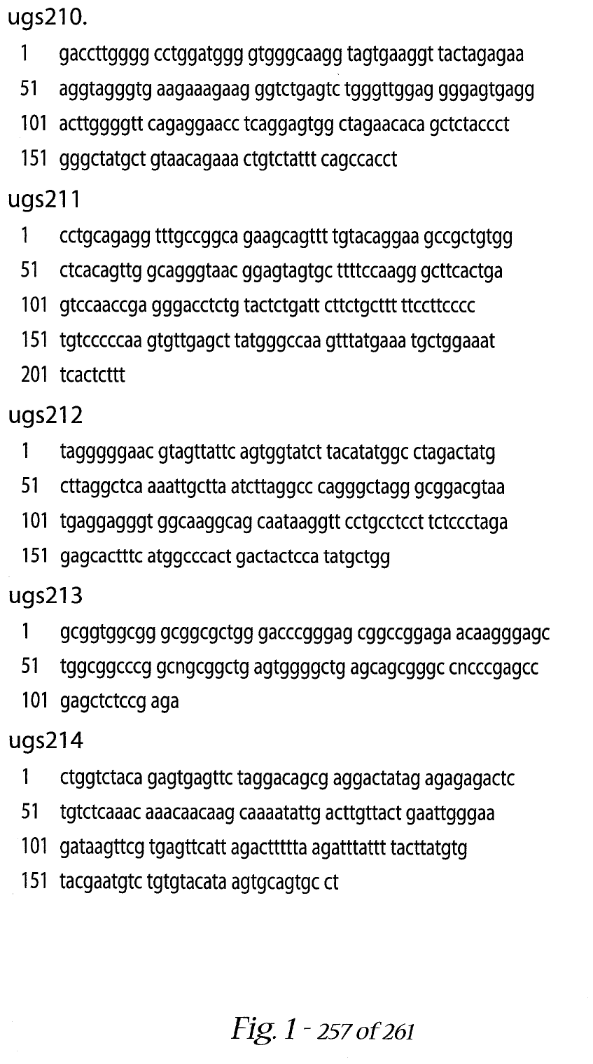

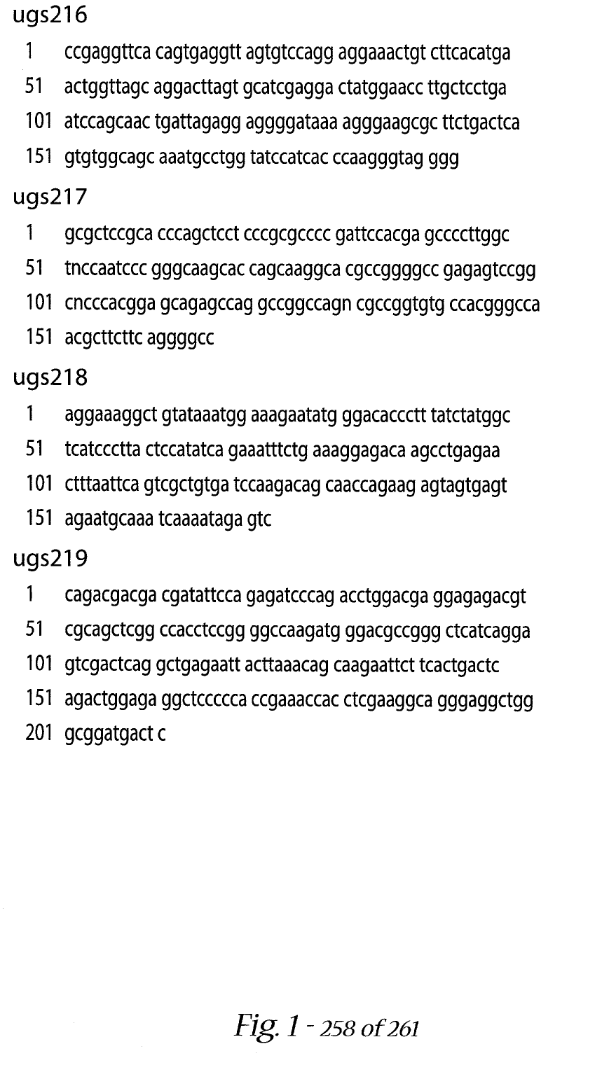

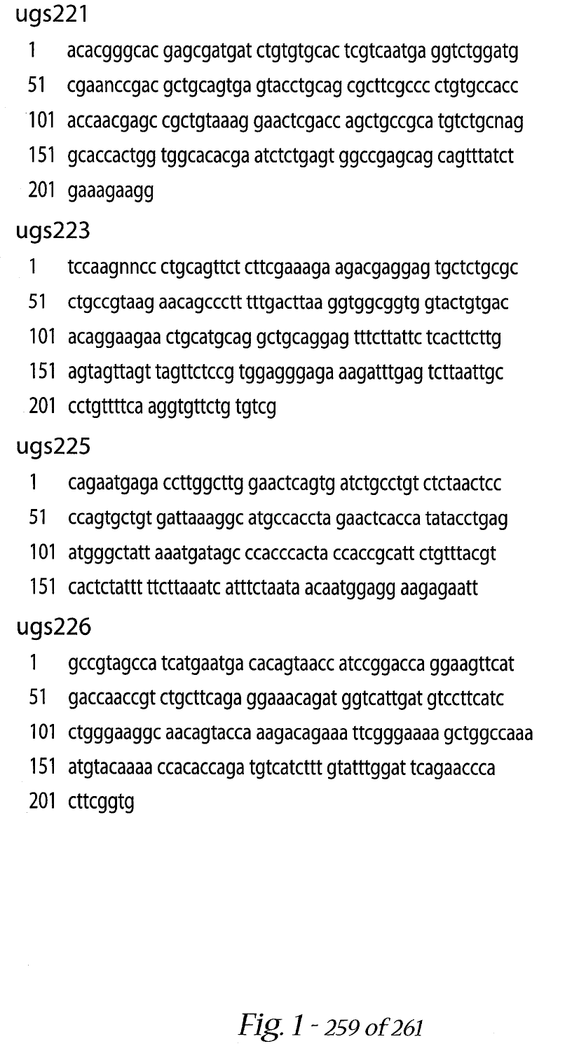

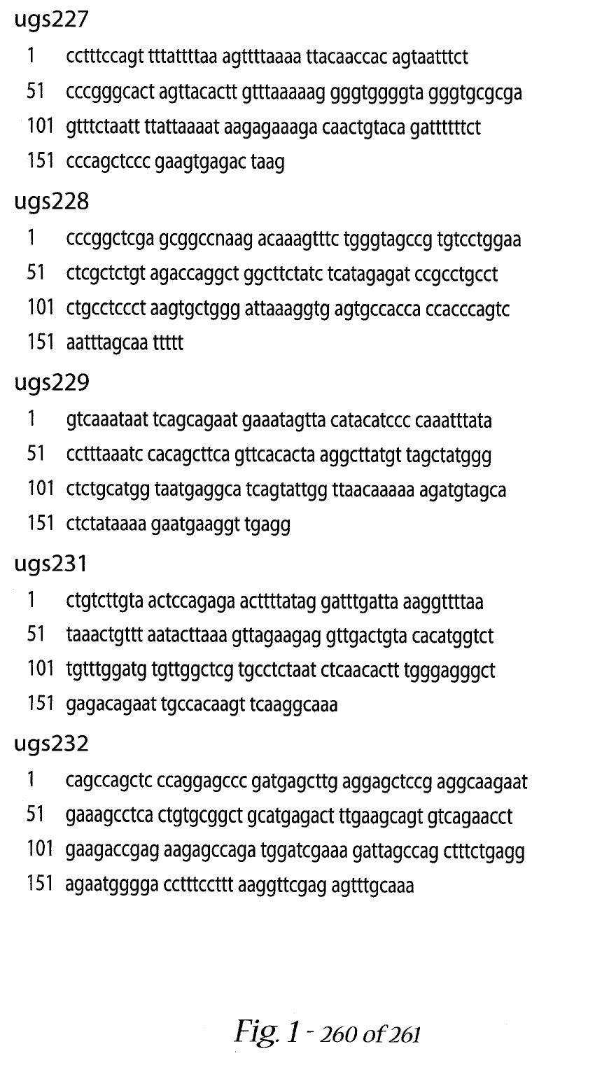

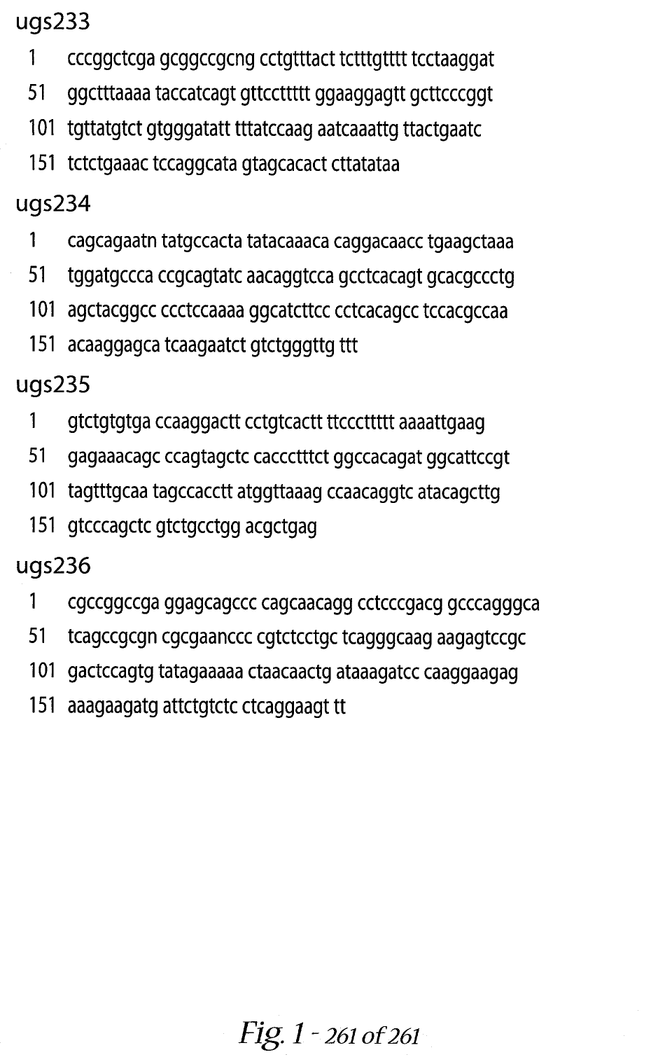

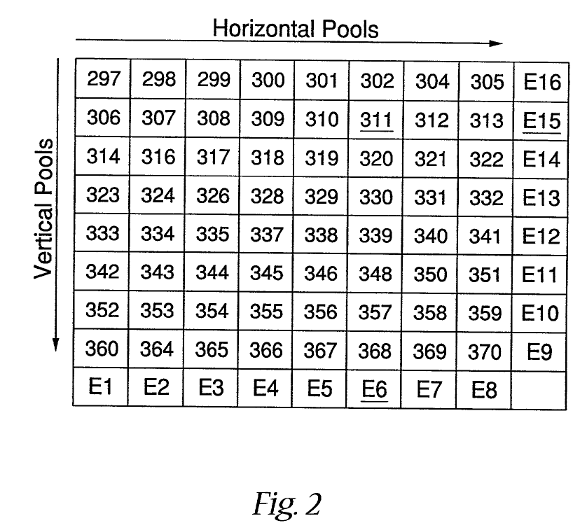

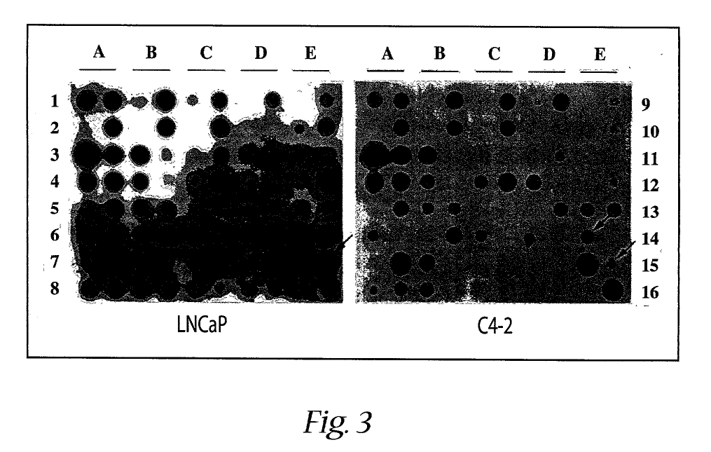

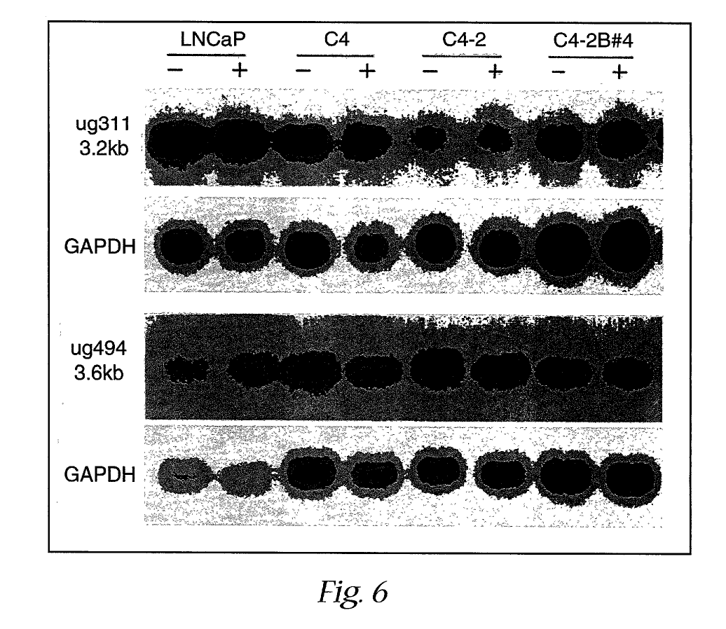

The 728 cloned and sequenced urogenital sinus expressed sequence tags (ESTs), representing 787 identified bacterial clones, total more than 330,000 bp of nucleotide sequence. These ESTs were first compared to the GenBank database with the following results: unique=64%; Known=28%; Moderate homology=5% with 3% vector sequences. The high complexity of the fetal library is comparable to the fetal heart findings of CC Lieu in Toronto. In order to narrow the focus to those fetal genes expressed during prostate cancer progression, a matrix blot format was developed (FIG. 1). In this format it is possible to screen 384 clones per filter using a 8×12 dot blot apparatus. The data obtained for 320 clones is depicted in FIG. 2. Using this grid matrix and probing duplicate filters simultaneously with LNCaP and C4-2 ³²p labeled cDNAs, 33 clones were identified from all 728 ESTs examined whose expression levels change dramatically between the two cell lines. The arrows shown in FIG. 2 indicate a pair of spots where the level of expression between LNCaP and C4-2 has dropped remarkably. By following the clone grid (FIG. 1, underlined) for the two E columns one can locate the spots corresponding to the increased signals. A clone's level of expression must change in at least two spots and be confirmed by RNA blot to be identified as increased (up-regulated) or decreased (down-regulated). As can be seen in the northern blot shown in FIG. 3, the clone designated UG311 has an elevated expression in LNCaP that decreases with increasing malignant potential, e.g. C4-2 by 5-7 fold. This particular clone is not regulated by androgens. In a similar fashion, other clones have been identified from these duplicate blots which are up regulated from the LNCaP to C4-2 in the human prostate cancer progression model. For example, FIG. 6 depicts the Northern blot for ug494, as well as for ug311, using the LNCaP (androgen dependent, non-tumorigenic) and lineage derived C4-2 (androgen-independent, tumorigenic metastatic to bone) cell line model. FIG. 6 shows that the fetal gene-derived EST ug494 is up-regulated in the C4-2 cell line compared to the LNCaP progression prostate cancer model cell line. These results form the basis of the experimental design described in more detail in Example 1 to completely characterize the UG311 EST by cloning and expressing UG311 EST in both bacterial systems for antibody development and in mammalian cell lines to determine their ability to modify the behavior of the LNCaP-C4-2 human prostate cancer progression model.

Tables 1-7 represent the subtractive analysis of homology determinations for all of the 728 cDNA clones as performed against the various databases. The asterisk represents a potentially differentially expressed UGS cDNA clone. See Table 7 for a summary of potentially differentially expressed UGS cDNA clones.

In particular, Table 1 presents the results of the library analysis of 787 cDNA UGS-derived ESTs using the Swissprot database.

TABLE 1
Results of the library analysis of 787 cDNA UGS-derived ESTs
using the Swissprot database

SWISSPROT
release 35 plus updates with fasty3_t -H -n -w 80 -m 6 KTUP = 2;
Unknown Eθ> .1 = 581; Known Eθ< 1.00E-6 = 168;
Uncertains(inbetween) = 66

| | | | |
|---|---|---|---|
| Clone | Prot. Locus | Acc. # | Identity |

GTPases

| | | | |
|---|---|---|---|
| ua1e1f | RB5C_CANFA | P51147 | canis familiaris (dog). ras-related protein Rab-5c. |
| | | | 10/ (216 aa) 8.4e-32 |
| ua1h3f | KPCI_HUMAN | P41743 | homo sapiens (human). protein kinase c, iota |
| | | | type (ec 2 (587 aa) 1e-51 |
| ug114rcon | RB5B_HUMAN | P35239 | homo sapiens (human), and mus musculus(mouse). |
| | | | ras-related protein Rab-5b (215 aa) 2.4e-29 |
| ug126 | CYA6_MOUSE | Q01341 | mus musculus (mouse). adenylate cyclase, type vi |
| | | | (ec 4. (1165 aa) 1.7e-24 |
| ug139 | MMR1_MOUSE | P36916 | mus musculus (mouse). possible gtp-binding protein |
| | | | mmr1 (430 aa) 2.6e-26 |
| *ug307cons | GBLP_HUMAN | P25388 | homo sapiens (human), mus musculus (mouse) |
| | | | guanine nucleotide binding protein (317 aa) 3.2e-57 |
| *ug308t | GBLP_HUMAN | P25388 | homo sapiens (human), mus musculus (mouse) |
| | | | guanine nucleotide binding protein (317 aa) 8.8e-55 |
| ug326 | GBLP_HUMAN | P25388 | homo sapiens (human), mus musculus (mouse) |
| | | | guanine nucleotide binding protein (317 aa) 4.6e-61 |

Protein Kinases

| | | | |
|---|---|---|---|
| ug014rcon | 143E_ARATH | P48347 | arabidopsis thaliana (mouse-ear cress). 14-3-3-like |
| | | | protein GF14 epsilon (254 aa) 1e-19 |
| ug265 | KPCE_MOUSE | P16054 | mus musculus (mouse). protein kinase c, epsilon type |
| | | | (NPKC-epsilon) (737 aa) 3e-28 |
| ug365 | 143Z_RAT | P35215 | rattus norvegicus (rat), and mus musculus 14-3-3 |
| | | | protein (245 aa) 5.8e-50 |
| ug425 | 143T_HUMAN | P27348 | homo sapiens (human). 14-3-3 protein tau (14-3-3 |
| | | | protein theta)(14-3-3 protein T-cell) (245 aa) 6.5e-18 |
| ug479 | P153_HUMAN | P53807 | h phosphatidylinositol-4-phosphate 5-kinase type iii |
| | | | (e(406 aa) 3.3e-18 |
| ugs020 | KC21_RAT | P19139 | rattus norvegicus (rat). casein kinase ii, alpha chain |
| | | | (c(391 aa) 2.2e-20 |
| *ugs186oft | CLK1_MOUSE | P22518 | mus musculus (mouse). protein kinase clk (ec 2.7.1.-) ( |
| | | | (483 aa) 5.7e-08 (see also clk-4 AF033566 (1549 nt) |
| | | | 9.7e-39) |

Structural Proteins

| | | | |
|---|---|---|---|
| ug023rcon | AP19_MOUSE | Q00382 | m clathrin coat assembly protein ap19 (clathrin coat |
| | | | assembly protein) (158 aa) 2.5e-27 |
| ug049rcon | YAD5_YEAST | P39730 | saccharomyces cerevisiae (baker's yeast). 112.3 kd |
| | | | protein PYK1-SNC21 intergenic region (1002 aa) |
| | | | synaptobrevins homolog. Vesicle fusion and |
| | | | exocytosis regulator. 2.7e-29 |
| ug052rcon | CA13_RAT | P13941 | rattus norvegicus (rat). collagen alpha 1(iii) chain (fra |
| | | | (636 aa) 1.4e-24 |
| ug061rcon | CALX_MOUSE | P35564 | mus musculus (mouse). calnexin precursor. 11/1995 |
| | | | (591 aa) 1.6e-27 |
| ug0982rcon | CA13_RAT | P13941 | rattus norvegicus (rat). collagen alpha 1(iii) chain (fra |
| | | | (636 aa) 1.2e-48 |
| ug116rcon | DREB_CHICK | P18302 | gallus gallus (chicken). drebrins e1 and e2. 6/1994 |
| | | | (607 aa) 3.7e-07 |
| ug136rcon | KELC_DROME | Q04652 | drosophila melanogaster (fruit fly). ring canal protein |
| | | | (intercell comm.: cytoplasm exchge regulator) (689 aa) |
| | | | 3e-10 |
| ug164rcon | VIME_MOUSE | P20152 | mus musculus (mouse). vimentin. 10/1996 (465 aa) |
| | | | 2.9e-29 |
| ug170rcon | FBLC_MOUSE | Q08878 | mus musculus (mouse). fibulin-1, isoform c precursor |
| | | | (b (685 aa) 8.4e-51 |
| ug256 | ANX5_MOUSE | P48036 | m annexin v (lipocortin v) (endonexin ii) calphobindin |
| | | | (319 aa) 1.7e-19 |
| ug284 | ANKC_HUMAN | Q01485 | homo sapiens (human). ankyrin, brain variant 2 |
| | | | (ankyrin (1839 aa) 2.7e-08 |
| ug297 | ATCP_RAT | P11505 | rattus norvegicus (rat). calcium-transporting atpase |
| | | | plasma membrane calcium pump (1176 aa) 6.5e-40 |
| ug382 | CTNA_MOUSE | P26231 | mus musculus (mouse). alpha-catenin (102 kd |
| | | | cadherin-as (906 aa) 6.4e-31 |
| ug401 | VP36_CANFA | P49256 | canis familiaris (dog). vesicular integral-membrane pro |
| | | | (356 aa) 6.5e-43 |
| ug415 | MYST_HUMAN | P35749 | homo sapiens (human). myosin heavy chain, smooth |
| | | | muscle (1086 aa) 2.9e-12 |
| ug416 | SPCB_HUMAN | P11277 | homo sapiens (human). spectrin beta chain, |
| | | | erythrocyte. (2137 aa) 5.2e-34 |
| ug427 | TBB1_PEA | P29500 | pisum sativum (garden pea). tubulin beta-1 chain. |
| | | | 10/1994 (450 aa) 2.8e-14 |
| ug420 | NFM_MOUSE | P08553 | mus musculus (mouse). neurofilament triplet m protein |
| | | | (1 (848 aa) 1.9e-30 |
| ug481 | NFM_MOUSE | P08553 | mus musculus (mouse). neurofilament triplet m protein |
| | | | (1 (848 an) 1.9e-30 |
| ug517 | GPCK_MOUSE | P51655. | mus musculus (mouse). k-glypican precursor 10/1996 |
| | | | (557 aa) 1.6e-43 |
| ug523 | KIF1_MOUSE | P33173 | mus musculus (mouse). kinesin-like protein kif1 |
| | | | (fragme (147 aa) 1.5e-25 |
| ugs016 | PGS2_MOUSE | P28654 | mus musculus (mouse). bone proteoglycan ii precursor |
| | | | (p (354 aa) 5e-22 |
| ugs072 | TPM_RAT | P18344 | rattus norvegicus (rat). tropomyosin alpha chain, brain- |
| | | | 3 (245 aa) 2.3e-23 |
| ugs117 | TALI_MOUSE | P26039 | mus musculus (mouse). talin. 2/1994 (2541 aa) |
| | | | 7.1e-20 |
| ugs160 | EG5_HUMAN | P52732 | homo sapiens (human). kinesin-like protein eg5. |
| | | | 10/1996 (1057 aa) 1.2e-17 |

Growth Factors, Cytokines & Binding Proteins

| | | | |
|---|---|---|---|
| ua2h6f | IBP2_MOUSE | P47877 | mus musculus (mouse). insulin-like growth factor |
| | | | bindin (305 aa) 2.5e-35 |
| ug130 | 1BP5_MOUSE | Q07079 | inns musculus (mouse). insulin-like growth factor |
| | | | bindin (271 aa) 3.8e-50 |
| ug150rcon | TYB4_MOUSE | P20065 | mus musculus (mouse). thymosin beta-4. 5/1992 (50 |
| | | | aa) 2.1e-14 |
| ug264 | AAAT_MOUSE | P51912 | mus musculus (mouse). insulin-activated amino acid |
| | | | tran (553 aa) 9.7e-33 |
| ug300 | PC4_MOUSE | P19182 | mus musculus (mouse). interferon-related protein pc4 |
| | | | (tp (449 aa) 1.2e-24 |
| ug324 | ENDR_BOVIN | P07106 | bos taurus (bovine). endozepine-related protein precurs |
| | | | (533 aa) 3.9e-16 |
| ug347 | MFGM_MOUSE | P21956 | mus musculus (mouse). milk fat globule-egf factor 8 |
| | | | pre (463 aa) 4e-10 |
| ug394 | THA1_MOUSE | P16416 | mus musculus (mouse). thyroid hormone receptor |
| | | | alpha-1. (410 aa) 6.7e-25 |

Ribosomal Proteins and Translation

| | | | |
|---|---|---|---|
| ug029rcon | RL11_RAT | P25121 | rattus norvegicus (rat). 60s ribosomal protein 111. |
| | | | 6/199 (177 aa) 6.4e-38 |
| ug068rcon | RL13_MOUSE | P47963 | mus musculus (mouse). 60s ribosomal protein 113 |
| | | | (a52). (212 aa) 2.1e-37 |
| ug086rcon | KS61_MOUSE | P18653 | mus musculus (mouse). ribosomal protein s6 kinase ii |
| | | | al (724 aa) 3.6e-10 |
| ug129 | RL2A_RAT | P18445 | rattus norvegicus (rat). 60s ribosomal protein 127a. |
| | | | 11/1 (147aa) 1.4e-46 |
| ug127 | SR72_CANFA | P33731 | canis familiaris (dog). signal recognition particle 72 |
| | | | (670 aa) 7.4e-48 |
| ug149rcon | RL5_RAT | P09895 | rattus norvegicus (rat). 60s ribosomal protein 15. |
| | | | 10/1996 (296 aa) 1.4e-52 |
| ug172rcon | RL13_MOUSE | P47963 | mus musculus (mouse). 60s ribosomal protein 113 |
| | | | (a52). (212 aa) 5.7e-44 |
| ug187rcon | RL2A_MOUSE | P14115 | mus musculus (mouse). 60s ribosomal protein 127a |
| | | | (129). (147 aa) 1.3e-44 |
| ug194 | RL31_HUMAN | P12947 | homo sapiens (human), and rattus norvegicus (rat). 60s |
| | | | (125 aa) 1.5e-36 |
| ug290 | RS15_HUMAN | P11174 | homo sapiens (human), mus musculus (mouse), rattus |
| | | | norv ribosomal protein S15 (144 aa) 4e-43 |
| ug303 | EF1A_MOUSE | P10126 | mus musculus (mouse). elongation factor 1-alpha(ef-1- |
| | | | a (462 aa) 6.1e-62 |
| *ug334 | SR14_MOUSE | P16254 | mus musculus (mouse). signal recognition particle 14 |
| | | | kd (110 aa) 6.9e-42 |
| *ug354cons | RLA2_HUMAN | P05387 | homo sapiens (human). 60s acidic ribosomal protein |
| | | | p2. (115 aa) 1.2e-29 |
| ug381 | EF1B_HUMAN | P24534 | homo sapiens (human). elongation factor 1-beta (ef-1- |
| | | | beta) (224 aa) 8.8e-39 |
| ug460 | RS23_HUMAN | P39028 | homo sapiens (human), and rattus norvegicus (rat). 40s |
| | | | ribosomal protein S23 (143 aa) 1.9e-11 |
| ug475 | RS24_HUMAN | P16632 | homo sapiens (human), rattus norvegicus (rat), mus |
| | | | musc 40s ribosomal protein S24 (S19) (133 aa) le-33 |
| ug502 | SYHH_HUMAN | P49590 | homo sapiens (human). histidyl-trna synthetase |
| | | | homolog (506 aa) 5.5e-13 |
| ugs059 | RS6_HUMAN | P10660 | homo sapiens (human), rattus norvegicus (rat), and |
| | | | mus m ribosomal protein S6 (phosphoprotein |
| | | | NP33)(249 aa) 1.1e-24 |
| ugs095 | RSP4_HUMAN | P08865 | homo sapiens (human). 40s ribosomal protein sa (p40) |
| | | | (34/67 kDa laminin binding protein) (295 aa) 6.3e-23 |
| ugs114 | S61A_RAT | P38378 | rattus norvegicus (rat). protein transport protein sec61p |
| | | | (ribosomal associated transport protein) (475 aa) |
| | | | 2.1e-23 |
| ugs142 | RL7A_MOUSE | P12970 | mus musculus (mouse). 60s ribosomal protein 17a |
| | | | (surfeit locus protein 3) (265 aa) 1.4e-13 |
| ugs188 | RS18_HUMAN | P25232 | homo sapiens (human), rattus norvegicus (rat), and |
| | | | mus musculus 40S ribosomal protein S18 (KE3) (152 |
| | | | aa) 1.3e-21 |
| ugs226 | RS24_XENLA | P02377 | xenopus laevis (african clawed frog). 40s ribosomal |
| | | | protein S24 (S19) (132 aa) 4e-27 |

Transcription Factors

| | | | |
|---|---|---|---|
| ua1a2 | SON_HUMANP | 18583 | homo sapiens (human) son protein (son3). DNA |
| | | | binding protein w/mos and myc homology 11/1995 |
| | | | (1523 aa) 2.3e-13 |
| ug027rcon | PUR_MOUSE | P42669 | mus musculus (mouse). transcriptional activator |
| | | | protein (321 aa) 3.1e-07 |
| ug087rcon | TYY1_MOUSE | Q00899 | mus musculus (mouse). transcriptional repressor |
| | | | protein (414 aa) 2.5e-33 |
| ug113rcn1o | POL2_MOUSE | P11369 | mus musculus (mouse). retrovirus-related pol |
| | | | polyprotei (1300 aa) 8.8e-36 |
| ug228 | ZN83_HUMAN | P51522 | homo sapiens (human). zinc finger protein 83 |
| | | | (zinc fing (428 aa) 6.4e-08 |
| ug243 | POL2_MOUSE | P11369 | mus musculus (mouse). retrovirus-related pol |
| | | | polyprotei (1300 aa) 7.1e-51 |
| ug249 | POL2_MOUSE | P11369 | mus musculus (mouse). retrovirus-related pol |
| | | | polyprotei (1300 aa) 1.8e-10 |
| ug271 | CABA_MOUSE | Q99020 | mus musculus (mouse). carg-binding factor-a (cbf-a). |
| | | | 11 (285 aa) 3.5e-08 |
| *ug277t | HXAD_AMBME | P50210 | ambystoma mexicanum (axolotl). homeotic protein |
| | | | hox-a13 (107 aa) 1.2e-34 (other locus 1399859 Acc |
| | | | #U59322) |
| ug289 | SN21_HUMAN | P28370 | homo sapiens (human). possible global transcription |
| | | | activator (976 aa) 1.9e-09 |
| ug313 | POL2_MOUSE | P11369 | mus musculus (mouse). retrovirus-related pol |
| | | | polyprotei (1300 aa) 2.7e-37 |
| ug367 | ETF_MOUSE | P48301 | mus musculus (mouse). embryonic tea domain- |
| | | | containing factor (445 aa) 2.1e-23 |
| ug486 | CL36_RAT | P52944 | rattus norvegicus (rat). lim protein clp36. (contains |
| | | | homeodomain of lin-11) 10/1996 (327 aa) 2e-24 |
| ugs101 | POL2_MOUSE | P11369 | mus musculus (mouse). retrovirus-related pol |
| | | | polyprotei (1300 aa) 5.8e-23 |

Mitochondrial

| | | | |
|---|---|---|---|
| ug002rcon | ATP6_MOUSE | P00848 | mus musculus (mouse). atp synthase a chain (ec |
| | | | 3.6.1.34 (226 aa) 1.2e-52 |
| ug007rcon | CYB_MOUSE | P00158 | mus musculus (mouse). cytochrome b (ec 1.10.2.2). |
| | | | 3/1992 (381 aa) .8e-85 |
| ug045con | NU5M_MOUSE | P03921 | mus musculus (mouse). nadh-ubiquinone |
| | | | oxidoreductase ch (607 aa) 5.1e-37 |
| ug063rcon | GR75_MOUSE | P38647 | mus musculus (mouse). mitochondrial stress-70 |
| | | | protein p (679 aa) 3.4e-31 |
| ug103rcon | ATP6_MOUSE | P00848 | mus musculus (mouse). atp synthase a chain (ec |
| | | | 3.6.1.34 (226 aa) 1.1e-19 |
| ug207 | NU5M_MOUSE | P03921 | mus musculus (mouse). nadh-ubiquinone |
| | | | oxidoreductase ch (607 aa) 8.9e-55 |
| ug296 | ATP6_MOUSE | P00848 | mus musculus (mouse). atp synthase a chain (ec |
| | | | 3.6.1.34 (226 aa) 7e-27 |
| ug336 | ATP6_MOUSE | P00848 | mus musculus (mouse). atp synthase a chain (ec |
| | | | 3.6.1.34 (226 aa) 7.2e-32 |
| ug363 | NU4M_MOUSE | P03911 | mus musculus (mouse). nadh-ubiquinone |
| | | | oxidoreductase ch (459 aa) 1.4e-46 |
| ug378 | ATPQ_RAT | P31399 | rattus norvegicus (rat). atp synthase d chain, |
| | | | mitochondr (160 aa) 2.8e-11 |
| ug489 | NU1M_MOUSE | P03888 | mus musculus (mouse). nadh-ubiquinone |
| | | | oxidoreductase ch (315 aa) 4.5e-61 |
| ug510 | COX3_MOUSE | P00416 | mus musculus (mouse). cytochrome c oxidase |
| | | | polypeptide (261 aa) 2.8e-11 |
| ugs064 | ATP6_MOUSE | P00848 | mus musculus (mouse). atp synthase a chain (ec |
| | | | 3.6.1.34 (226 aa) 1.1e-18 |
| ugs091 | NU6M_MOUSE | P03925 | mus musculus (mouse). nadh-ubiquinone |
| | | | oxidoreductase ch (172 aa) 3.7e-32 |
| ugs094 | ATP6_MOUSE | P00848 | mus musculus (mouse). atp synthase a chain (ec |
| | | | 3.6.1.34 (226 aa) 4.6e-19 |

RNA Splicing, Binding, RNPs, etc...

| | | | |
|---|---|---|---|
| ug072rcon | P68_HUMAN | P17844 | homo sapiens (human). p68 protein (rna helicase). |
| | | | 6/1994 (614 aa) 1.2e-16 |
| ug145 | HMT1_YEAST | P38074 | saccharomyces cerevisiae (baker's yeast). hnrnp |
| | | | arginine n-methyltransferase (348 aa) 4.2e-17 |
| ug225 | ROA1_MOUSE | P49312 | mus musculus (mouse). heterogeneous nuclear |
| | | | ribonucleop (319 aa) 3.7e-15 |
| ug293 | PSF_HUMAN | P23246 | homo sapiens (human). ptb-associated splicing factor |
| | | | (ps (707 aa) 1.3e-41 |
| ug310 | FUS_HUMAN | P35637 | homo sapiens (human). ma-binding protein fus/tls. |
| | | | 11/19 (526 aa) 1.7e-27 |
| *ug311icons | PSF_HUMAN | P23246 | homo sapiens (human). ptb-associated splicing factor |
| | | | (ps (707 aa) 1.7e-25 |
| ug391 | RSMB_MOUSE | P27048 | mus musculus (mouse). small nuclear |
| | | | ribonucleoprotein a (231 aa) 2.6e-25 |
| *ug485ors | RNPL_HUMAN | P98179 | homo sapiens (human). putative rna-binding protein |
| | | | rnpl (157aa) 3.1e-12 |
| ugs115 | UBIQ_HUMAN | P02248 | homo sapiens (human), bos taurus (bovine), |
| | | | UBIQUITIN (76 aa) 3e-14 |
| ugs128 | P68_HUMAN | P17844 | homo sapiens (human). p68 protein (rna helicase). |
| | | | 6/1994 (614 aa) 2.6e-13 |

Peptidases, Proteinases, Isomerases, Transferases

| | | | |
|---|---|---|---|
| *ug101rcon | DPP4_MOUSE | P28843 | mus musculus (mouse). dipeptidyl peptidase iv (ec |
| | | | 3.4.1) (760 aa) 5.7e-07 |
| ug153rcon | PPI1_MOUSE | P53810 | mus musculus (mouse). phosphatidylinositol (ptdins) |
| | | | transfer protein alpha (270 aa) 9.2e-26 |
| ug188rcon | NMT_HUMAN | P30419 | homo sapiens (human). glycylpeptide n- |
| | | | tetradecanoyltransferase (peptide N- |
| | | | myristoyltransferase) (NMT) (416 aa) 1e-51 |
| ug211 | COGT_MOUSE | P53690 | mus musculus (mouse). matrix metalloproteinase-14 |
| | | | precu (582 aa) 3.2e-51 |
| *ug335 | NEP_RAT | P07861 | rattus norvegicus (rat). neprilysin (ec 3.4.24.11) |
| | | | (neutral endopeptidase) (749 aa) 5e-20 |
| ug458 | VKGC_HUMAN | P38435 | homo sapiens (human). vitamin k-dependent gamma |
| | | | glutamyl-carboxylase (758 aa) 1.7e-34 |
| ugs030 | PUR6_RAT | P51583 | r multifunctional protein ade2 |
| | | | (amidophosphoribosyltransferase) Cell cycle |
| | | | dependent regulation (425 aa) 1.7e-16 |
| ugs123 | PDI_MOUSE | P09103 | m protein disulfide isomerase precursor (pdi). (509 aa) |
| | | | 5e-11 |
| ugs180 | AMP2_RAT | P38062 | rattus norvegicus (rat). methionine aminopeptidase 2 |
| | | | (478 aa) 7.2e-27 |
| ugs190 | FUCO_HUMAN | P04066 | homo sapiens (human). tissue alpha-1-fucosidase |
| | | | precurs (Lysosomal storage) (461 aa) 6.8e-25 |

Chromosomal Associated

| | | | |
|---|---|---|---|
| ug040rcon | RCC_MESAU | P23800 | mesocricetus auratus (golden hamster). regulator of |
| | | | chromosomal condensation (421 aa) 4.8e-07 |
| ugs010 | H33_HUMAN | P06351 | homo sapiens (human), mus musculus (mouse), rattus |
| | | | norve histone H3.3 (H3b) (135 aa) 4.3e-18 |
| ugs146 | TPR_HUMAN | P12270 | homo sapiens (human). nucleoprotein tpr. 10/1996 |
| | | | (2349 aa) 1.9e-15 |

Heat Shock, Chaperones, Stress-Induced

| | | | |
|---|---|---|---|
| ugo42con | HS9B_MOUSE | P11499 | mus musculus (mouse). heat shock protein hsp 84 |
| | | | (tumor specific transplantation antigen) (723 aa) 3.7e- |
| | | | 51 |
| ug356 | HS7C_RAT | P08109 | rattus norvegicus (rat), and mus musculus (mouse). |
| | | | heat shock cognate 71kDa (646 aa) 6.3e-58 |

Neural Specific

| | | | |
|---|---|---|---|
| ug379 | HIPP_HUMAN | P41211 | homo sapiens (human). neuron specific calcium- |
| | | | binding protein hippocalcin (BDR-2) (192 aa) 9.2e-09 |
| ugs023 | NED4_MOUSE | P46935 | mus musculus (mouse). nedd-4 protein (ec6.3.2.-) |
| | | | neural precursor cell protein (frag (957 aa) 4.8e-19 |

Hypothetical

| | | | |
|---|---|---|---|
| *ug093rcon | YO11_MOUSE | P11260 | mus musculus (mouse). hypothetical protein orf-1137. |
| | | | (L1Md domain protein, repetitive element retroposon- |
| | | | like) 7/ (379 aa) 4.4e-46 |
| ug095rcon | YJZ4_YEAST | P47095 | saccharomyces cerevisiae (baker's yeast). hypothetical |
| | | | (244 aa) 8.3e-18 |
| ug309 | YNK7_YEAST | P53930 | saccharomyces cerevisiae (baker's yeast). hypothetical |
| | | | (226 aa) 2.5e-14 |
| ug412 | YCFB_HAEIN | P44551 | haemophilus influenzae. hypothetical protein hi0 174. |
| | | | 10 (418 aa) 6.5e-11 |

Nucleotide metabolism (Cytosolic)

| | | | |
|---|---|---|---|
| ug084rcon | THIO_MOUSE | P10639 | inns musculus (mouse). thioredoxin (atl-derived factor) |
| | | | ribont-deoxyribont converter and general reducer (104 |
| | | | aa) 2.3e-21 |
| ug413 | ARF5_HUMAN | P26437 | homo sapiens (human), and rattus norvegicus (rat). |
| | | | adp-ribosylation factor 5 (179 aa) 5.2e-07 |

Unknown

| | | | |
|---|---|---|---|
| ug480 | IGEB_MOUSE | P03975 | mus musculus (mouse). IgE-binding protein. 4/1988 |
| | | | (557 aa) 1.5e-24 |
| ugs044 | TLM_MOUSE | P17408 | mus musculus (mouse). tlm protein (tlm oncogene). |
| | | | 12/199 (317aa)1.4e-07 |

Vector Associated (Tet-R/Beta-gal)

| | | | |
|---|---|---|---|
| ug016_38_80 | TER1_ECOLI | P03038 | escherichia coli , tetracycline repressor protein class |
| | | | (216 aa) 4.7e-27 |
| ug060 | TER1_ECOLI | P03038 | escherichia coli. tetracycline repressor protein class |
| | | | (216 aa) 1.8e-32 |
| ug100rcon | TER1_ECOLI | P03038 | escherichia coli. tetracycline repressor protein class |
| | | | (216 aa) 9.5e-08 |
| ug108rcon | TER1_ECOLI | P03038 | escherichia coli. tetracycline repressor protein class |
| | | | (216 aa) 4.6e-31 |
| ug122rcon | TER1_ECOLI | P03038 | escherichia coli. tetracycline repressor protein class |
| | | | (216 aa) 1.7e-30 |
| ug165rcon | BGAL_ECOLI | P00722 | escherichia coli. beta-galactosidase (ec 3.2.1.23) (lac |
| | | | (1023 aa) 3.1e-13 |
| ug166rcon | TER1_ECOLI | P03038 | escherichia coli. tetracycline repressor protein class |
| | | | (216 aa) 5.2e-35 |
| ug193 | TER1_ECOLI | P03038 | escherichia coli. tetracycline repressor protein class |
| | | | (216 aa) 2.2e-24 |
| ug199 | TER1_ECOLI | P03038 | escherichia coli. tetracycline repressor protein class |
| | | | (216 aa)3.5e-11 |
| ug204 | TER1_ECOLI | P03038 | escherichia coli. tetracycline repressor protein class |
| | | | (216 aa) 2.2e-23 |
| ug215 | TER1_ECOLI | P03038 | escherichia coli. tetracycline repressor protein class |
| | | | (216 aa) 1.5e-12 |
| ug231 | TER1_ECOLI | P03038 | escherichia coli. tetracycline repressor protein class |
| | | | (216 aa) 4.8e-27 |
| ug235 | TER1_ECOLI | P03038 | escherichia coli. tetracycline repressor protein class |
| | | | (216 aa) 1e-29 |
| ug236 | TER1_ECOLI | P03038 | escherichia coli. tetracycline repressor protein class |
| | | | (216 aa) |
| ug268 | TER1_ECOLI | P03038 | escherichia coli. tetracycline repressor protein class |
| | | | (216 aa) 4.5e-16 |
| ug283 | TER1_ECOLI | P03038 | escherichia coli. tetracycline repressor protein class |
| | | | (216 aa) 1.1e-31 |
| *ug316cons | TER1_ECOLI | P03038 | escherichia coli. tetracycline repressor protein class |
| | | | (216 aa) 3e-20 |
| ug327 | TER1_ECOLI | P03038 | escherichia coli. tetracycline repressor protein class |
| | | | (216 aa) 5.1e-34 |
| ug349 | TER1_ECOLI | P03038 | escherichia coli. tetracycline repressor protein class |
| | | | (216 aa) 8.7e-31 |
| ug362 | TER1_ECOLI | P03038 | escherichia coli. tetracycline repressor protein class |
| | | | (216 aa) 2.3e-32 |
| ug375 | TER1_ECOLI | P03038 | escherichia coli. tetracycline repressor protein class |
| | | | (216 as) 9e-29 |
| ug386 | TER1_ECOLI | P03038 | escherichia coli. tetracycline repressor protein class |
| | | | (216 aa) 2.3e-23 |
| ug389 | TER1_ECOLI | P03038 | escherichia coli. tetracycline repressor protein class |
| | | | (216 aa) 3e-27 |
| ugs015 | TER1_ECOLI | P03038 | escherichia coli. tetracycline repressor protein class |
| | | | (216 aa) 7.6e-13 |

Table 2 presents the results of the library analysis of 787 cDNA UGS-derived ESTs using the GENPEPT translated protein database (rel 102.0).

TABLE 2
Results of the library analysis of 787 cDNA UGS-derived ESTs
using the GENPEPT translated protein database (rel 102.0)

GENPEPT
Translated Protein Database (rel 102.0); Additional known
Eθ < 1.00E-6 = 28

| | | | |
|---|---|---|---|
| Clone | Prot. Locus | Acc. # | Identity |

Protein Kinases

| | | | |
|---|---|---|---|
| ug135 | 1008544 | U35113 | breast adenocarcinoma metastasis- |
| | | | associated gene (contains SH3 |
| | | | domains) Homo sapiens (715aa) |
| | | | 2.5e-14 |

Membrane/Structural Proteins

| | | | |
|---|---|---|---|
| ug001rcon | 1228041 | D83779 | contains 9 hydrophobic domains. |
| | | | ÄHomo sapiens AÅ(1356 aa) 3.8e-66 |
| | | | the KIAA0195 gene is expressed |
| | | | ubiquitously.; the KIAA0195 protein |
| | | | retains 9 hydrophobic domains. |
| ug202 | 1109847 | U41538 | R04E5.6 gene product Caenorhabditis |
| | | | elegans (430 aa) 3.3e-14 Similar to |
| | | | cytoplasmic intermeidate filament |
| | | | protein” (chromosome III) |
| ug244 | 165704 | M76233 | Rabbit smooth muscle myosin light |
| | | | chain kinase mRNA, comp (1147 aa) |
| | | | 2.1e-18 |
| ugs112 | 1707522 | Y08612 | 88kDa nuclear pore complex protein |
| | | | ÄHomo (741 aa) 2.6e-22 |

Growth Factors, Cytokines & Binding
Proteins

| | | | |
|---|---|---|---|
| ug094rcon | 1480110 | X99643 | HP1-BP38 protein Mus musculus |
| | | | (TIF-like molecule) nuclear receptor |
| | | | ligand binding domain interactive |
| | | | protein (852 aa) 2.2e-40 |

Transcription Factors

| | | | |
|---|---|---|---|
| ug279 | 1203987 | L40331 | homologuous to yeast silent |
| | | | information regulatory 2 prot |
| | | | (381 aa) 6.1e-07 |
| ug374 | 431953 | X76302 | nucleic acid binding protein Homo |
| | | | sapiens (163 aa) 1.1e-08 |

Mitochondrial

| | | | |
|---|---|---|---|
| ug449 | 1332384 | M27315 | Rattus norvegicus cytochrome c |
| | | | oxidase subunit I Rattus (514 aa) |
| | | | 2.5e-38 |

RNA Splicing, Binding, RNPs, etc...

| | | | |
|---|---|---|---|
| ug019rcon | 1374782 | D85414 | possible ubiquitin protein ligase Mus |
| | | | musculus (957 aa) 5.5e-05 |
| ug168rcon | 619302 | S72641 | RNA-binding protein = Merc |
| | | | äalternatively spliced (296 aa) |
| | | | 1.1e-28 |
| ug081rcon | 603949 | D43947 | KIAA0100 is a human counterpart of |
| | | | mouse el gene. Homo sa (2092 aa) |
| | | | 3.5e-55 |

Peptidases, Proteinases, Isomerases,
Transferases

| | | | |
|---|---|---|---|
| ualc6f | 1483249 | Z78012 | C52E4.6 Caenorhabditiselegans 23S |
| | | | protase regulatory subnunit 7” |
| | | | “Alpha 1,2 mannosidase”, |
| | | | “Cathepsin-like cysteine protease”, |
| | | | “Guanylate cyclase”,“HTG”, “Low- |
| | | | density lipoprotein receptor”, |
| | | | “Macrophage migration inhibitory |
| | | | factor like”, “Small nuclear |
| | | | ribonucleoprotein”}(534 aa) 2.4e-29 |
| ug062rcon | 1240019 | Z70287 | R09E10.7 Caenorhabditis elegans |
| | | | 2.2mb of chromoIII “HTG”, “Long- |
| | | | chain-fatty-acid-CoA ligase”, |
| | | | “Protein-tyrosine phosphatase”} |
| | | | (1791 aa) 4.4e-13 |
| ug312 | 802105 | S74907 | PP1M M110 = protein phosphatase |
| | | | 1M 110 kda regula (976 aa) 1.5e-27 |
| ug329 | 1808596 | Y08826 | alkyl-dihydroxyacetonephosphate |
| | | | synthase (658 aa) 4e-38 |

Heat Shock, Chaperones, Stress-Induced

| | | | |
|---|---|---|---|
| ug111nov | 687844 | U21320 | contains TPR domain-like repeats |
| | | | Caenorhabditis elegans (molecular |
| | | | chaperone for HSP's and other) |
| | | | (1194 aa) 6.7e-32 |
| ug167rcon | 860712 | U28735 | coded for by C. elegans cDNA |
| | | | cm06e4; coded for by C. elegans |
| | | | (1493 aa) 9.9e-14 similar to killer |
| | | | toxin-resistance protein 5 |
| | | | (SP: KRE5_YEAST, P22023) |

Unknown

| | | | |
|---|---|---|---|
| ug337 | 1469878 | D63482 | The KIAA0148 gene product is |
| | | | related to KIAA0041 and KIAA |
| | | | (471 aa) 6.4e-13 (ESTs of human |
| | | | cell line KG-1) |
| *ug371f | 1480863 | U63332 | super cysteine rich protein; SCRP |
| | | | Homo sapiens (46 aa) 1.9e-08 |
| | | | (expression appears ubiquitois) |
| ugs137 | 532816 | U13876 | similar to S. cerevisiae hypothetical |
| | | | 240.3 kd protein in C. Elegans |
| | | | similar to MSH3 3′sequence. |
| | | | (2500 aa) 2.4e-08 |
| ugs153 | 1483249 | Z78012 | C52E4.6 Caenorhabditis elegans |
| | | | (Chromosome 5) (534 aa) 5.1e-11 |
| ugs206 | 291844 | L13434 | Human chromosome 3p21.1 gene |
| | | | sequence from lung cancer, complete |
| | | | cds., conceptual translation (256 aa) |
| | | | 1.2e-18 |

Tet-Repressor/βeta-gal Cloning Vector

| | | | |
|---|---|---|---|
| ug067rcon | 1132426 | U39779 | beta-galactosidase alpha polypeptide |
| | | | ÄCloning vector pTri |
| | | | (139 aa) 2.9e-10 |
| ug258 | 1132426 | U39779 | beta-galactosidase alpha polypeptide |
| | | | ÄCloning vector pTri |
| | | | (139 aa) 6.7e-09 |

Table 3 presents the results of the library analysis of 787 cDNA UGS-derived ESTs using the primate rodent GB103 database.

TABLE 3
Results of the library analysis of 787 cDNA UGS-derived ESTs
using the primate rodent GB103 database

GenBank GB103: Primate and Rodent divisions
fasta3_t -H -n -w 80 -m 6 -1 /seqlib/lib/fastlibs % PR 6

| | | | |
|---|---|---|---|
| Clone | Locus | Acc. # | Identity |

GTPases

| | | | |
|---|---|---|---|
| ug270 | | AF070603 | Homo sapiens clone 24584 beta-subunit signal |
| | | | transducing proteins (Gs/Gi) (1889 nt) 4.6e-55 |
| ug447 | HSEWSGAR | Y07848 | Homo sapiens EWS, gar22, rrp22 and bam22 |
| | | | genes. 5/98 (79468 nt) 7.3e-65 (Identification of |
| | | | new members of the Gas2 and Ras families in the |
| | | | 22q12 chromosome region.) |
| ug451 | HSRANBP5 | Y08890 | H.sapiens mRNA for Ran_GTP binding protein 5. |
| | | | 9/97 (4826 nt) 9.3e-100 |
| ugs149 | HSU50078 | U50078 | Human guanine nucleotide exchange factor p532 |
| | | | mRNA, complet (15171 nt) 1.6e-36 |
| ugs177 | HSU90268 | U90268 | Human Krit1 mRNA, complete cds. 6/97 (2004 |
| | | | nt) 2.6e-19 |

Protein Kinases

| | | | |
|---|---|---|---|
| ug195 | MUSPGK1PS2 | M23962 | Mus musculus phosphoglycerate kinase (Pgk1- |
| | | | ps2) processed (1753 nt) 3.1e-68 |
| *ug441ors | | AF027504 | Mus musculus putative membrane-associated |
| | | | guanylate kinase 1 (Mag (919 nt) 1.8e-2 |
| ugs110 | HSDAPK | X76104 | H.sapiens DAP-kinase mRNA. 4/97 (5910 nt) 7e- |
| | | | 11 (Very repetitive jdr) |
| ugs147 | MMU51866 | U51866 | Mus musculus casein kinase II alpha subunit |
| | | | mRNA, complete (1552 nt) 1.4e-49 |

Structural Proteins/ECM

| | | | |
|---|---|---|---|
| *ug102cons | RATCTTG | M80829 | Rat troponin T cardiac isoform gene, complete |
| | | | cds. 9/96 (19185 nt) 2.3e-12 (Highly repetitive |
| | | | jdr) |
| ug174rcon | MMU48797 | U48797 | Mus musculus astrotactin mRNA, complete cds. |
| | | | 5/96 (6863 nt) 1.4e-31 |
| ug206 | RATSTPBCB | D83349 | Rat mRNA for short type PB-cadherin, complete |
| | | | cds. 7/96 (4153 nt) 3.9e-16 |
| ug392 | | AF078705 | Mus musculus vascular adhesion protein-1 gene, |
| | | | complete cds. 9/98 (14357 nt) 2.7e-19 |
| ug465 | MUSCK15 | D16313 | Mouse cytokeratin 15 gene, complete cds. 3/96 |
| | | | (6149 nt) 4.7e-58 |
| ug498 | RRU04320 | U04320 | Rattus norvegicus Wistar alpha B-crystallin gene, |
| | | | complete (6806 nt) 6e-14 |
| ug521 | RATCRYG | M19359 | Rat gamma-crystallin gene cluster, encoding |
| | | | gamma-A (gamma 1 (54670 nt) 1.6e-09 |
| ug525 | MMSPARCR | X04017 | Mouse mRNA for cysteine-rich glycoprotein |
| | | | SPARC. 9/93 (2079 nt) 1.1e-131 |
| ugs217 | MUSCOL4A | J04448 | Mouse alpha 1 and 2 collagen type IV genes, 5′ |
| | | | end. 1/94 (1200 nt) 9.6e-29 |

Oncogenes/Tumor Suppressors/Apoptosis

| | | | |
|---|---|---|---|
| ug142 | | AF060868 | Mus musculus tumor susceptibility protein 101 |
| | | | (tsg101) gene, comp (33613 nt) 2.6e-12 |
| ug219 | | AF060868 | Mus musculus tumor susceptibility protein 101 |
| | | | (tsg101) gene |
| | | | gene, comp (33613 nt) 2.5e-13 |
| ug201 | MUSRRG | D10837 | Mus muculus rrg (ras recision gene) mRNA, |
| | | | tumor suppressor opposes ras action, partial |
| | | | sequence. 1 (1942 nt) 2.6e-104 |
| ug218 | HSU41635 | U41635 | Human 0S-9 precursor mRNA, complete cds. |
| | | | 8/96 (sarcoma associated gene) (2736 nt) 6.5e-34 |
| *ug503s | | AF017989 | Mus musculus secreted apoptosis related protein |
| | | | 1(Sarp1) mRNA, c (2031 nt) 8.3e-57 |
| ugs216 | MMU50850 | U50850 | Mus musculus retinoblastoma-related protein |
| | | | p130 mRNA, (4013 nt) 2e-48 |

Growth Factors, Cytokines & Binding Proteins

| | | | |
|---|---|---|---|
| ug140 | MMIGFIIE6 | X71922 | M.musculus gene for IGF-II, exon 6. 7/95 (3321 |
| | | | nt) 1.2e-116 |
| ug306 | MMTNFBG | Y00137 | Mouse tumor necrosis factor-beta (lymphotoxin) |
| | | | gene. 5/93 (3219 nt) 6.4e-13 (polyA jdr) |
| ug414 | MMIL5G | X06271 | Murine gene for interleukin 5 (eosinophil |
| | | | differentiation fac (6727 nt) 1.5e-12 |
| ug518 | | AF063020 | Homo sapiens lens epithelium-derived growth |
| | | | factor mRNA, complete (3377 nt) 2.7e-57 |
| ug522 | MMU06950 | U06950 | Mus musculus C57BL/6 lymphotoxin-beta, |
| | | | lymphotoxin-alpha, (Murine TNF-alpha?beta |
| | | | locus) (15213 nt) 4e-21 |

Growth Factor Induced

| | | | |
|---|---|---|---|
| ug077rcon | MUSDIP | D44443 | Mouse mRNA for dexamethasone induced |
| | | | apoptosis in T-cells, complete cds. 5 (573 nt) 1.3e-08 |
| ug111rcon | MUSDIP | D44443 | Mouse mRNA for dexamethasone induced |
| | | | apoptosis in T-cells, complete cds. 5 (573 nt) 1.7e-17 |
| ug124 | RNPTHR202 | X95079 | R.norvegicus mRNA for parathyroid hormone |
| | | | regulated sequen (202 nt) 1.4e-19 |
| ugs167 | MUSGRP784 | D78645 | Mouse mRNA for 78 kDa glucose-regulated |
| | | | protein, complete (2408 nt) 1.5e-27 |

Ribosomal Proteins and rRNA

| | | | |
|---|---|---|---|
| ug257 | MM45SRRNA | X82564 | M.musculus 45S pre rRNA gene. 4/96 (22118 nt) |
| | | | 1.6e-30 |
| ug325 | MM45SRRNA | X82564 | M.musculus 45S pre rRNA gene. 4/96 (22118 nt) |
| | | | 2.6e-67 |
| ug361 | MM45SRRNA | X82564 | M.musculus 45S pre rRNA gene. 4/96 (22118 nt) |
| | | | 4.1e-42 |
| ug444 | MM45SRRNA | X82564 | M.musculus 45S pre rRNA gene. 4/96 (22118 nt) |
| | | | 4.5e-88 |
| ugs005 | MMJ1PRO | Y00225 | Murine mRNA for J1 protein, yeast ribosomal |
| | | | protein L3 homol (1276 nt) 3.4e-13 |
| ugs038 | MM45SRRNA | X82564 | M.musculus 45S pre rRNA gene. 4/96 (22118 nt) |
| | | | 1.5e-46 |

Membrane Proteins/Receptors

| | | | |
|---|---|---|---|
| ug083rcon | MMHC135G15 | AF050157 | Mus musculus major histocompatibility locus |
| | | | class II re (79435 nt) 5.2e-10 |
| ug093f | MMAE000663 | AE000663 | Mus musculus TCR beta locus from bases 1 to |
| | | | 250611 (sec (79890 nt) 4.7e-11 (TCR = T cell |
| | | | receptor jdr) |
| ug119 | MMAE000664 | AE000664 | Mus musculus TCR beta locus from bases 250554 |
| | | | to 501917 (79704 nt) 1.8e-18 |
| ug131 | MMAE000665 | AE000665 | Mus musculus TCR beta locus from bases 501860 |
| | | | to 700960 (40877 nt) 1.8e-13 |
| ug133 | | AF100956 | Mus musculus major histocompatibility locus |
| | | | class II region; Fas- (79588 nt) 3e-06 |
| ug155rcon | MMHC438N12 | AF049850 | Mus musculus major histocompatibility locus |
| | | | class III r (70941 nt) 3.3e-19 |
| ug176rcon | MUSSVA | L44117 | Mus musculus (clone GSmSVA) seminal vesicle |
| | | | autoantigen gene, (5307 nt) 1.2e-09 (Highly |
| | | | repetitve, but it is in the right place jdr) |
| ug200 | MMHC135G15 | AF050157 | Mus musculus major histocompatibility locus |
| | | | class II re (79513 nt) 5.2e-13 |
| ug214 | MMU97066 | U97066 | Mus musculus sulfonylurea receptor 2B (SUR2) |
| | | | mRNA, Protein associated with potassium |
| | | | ATPase transporter (6081 nt) 5.5e-07 |
| ug222 | MUSLYT3A6 | M22070 | Mouse MHC class I T-cell surface antigen gene |
| | | | Lyt-3-a enco (1249 nt) 2.8e-15 |
| *ug254 | MUSBA | D82019 | Mouse gene for basigin, complete cds (exon 1-7). |
| | | | 2/97 (11763 nt) (Basigin, a new, broadly |
| | | | distributed member of the immunoglobulin |
| | | | superfamily, has strong homology with both the |
| | | | immunoglobulin V domain and the beta-chain of |
| | | | major histocompatibility complex class II |
| | | | antigen.)1/98 (1302 nt) 5.9e-95 |
| ug260 | | AF018261 | Rattus norvegicus EH domain binding protein |
| | | | Epsin mRNA, complete (calthrin mediated |
| | | | endocytosis) (2047 nt) 4.3e-11 |
| ug287 | MMZNT4S3 | AF004099 | Mus musculus zinc transporter (ZnT4) gene, |
| | | | fragment 3, important for Zn uptake and |
| | | | sequestration into endosome/lysosomal and |
| | | | synaptic vesicles (1371 nt) 1.1e-08 |
| ug369 | | AF007558 | Mus musculus hemocbromatosis (HFE) gene |
| | | | (Critical molecule involved in cellular iron |
| | | | homeosatsis. Related to MHC genes., complete |
| | | | cds. 2/98 (14000 nt) 1.5e-12 |
| ug376 | MUSBB2R | L27595 | Mus muscaris bradykinin B2 receptor (B2R) gene, |
| | | | complete cds (8934 nt) 1.5e-07 |
| ug454 | MMCLCNVI4 | AF030104 | Mus musculus putative chloride channel protein |
| | | | CLC6 (Clc (14925 nt) 7e-11 |
| ug459 | MMHC135G15 | AF050157 | Mus musculus major histocompatibility locus |
| | | | class II re (8222 nt) 2.7e-09 |
| ug462 | HUMZD39G09 | AF086249 | Homo sapiens full length insert cDNA clone |
| | | | ZD39G09. 8/9 (555 nt) 7.8e-12 Similar to |
| | | | INTEGRIN BETA-1 |
| ug468 | MMMMH461 | AF027865 | Mus musculus Major Histocompatibility Locus |
| | | | class II regi (79560 nt) 7.6e-110 |
| ug474 | | AF100956 | Mus musculus major histocompatibility locus |
| | | | class II region; Fas- (79856 nt) 3.7e-15 |
| *ug493ors | MMEZR | X60671 | M.musculus mRNA for ezrin. 8/96 (2701 nt) (A |
| | | | gene family consisting of ezrin, radixin and |
| | | | moesin. Its specific localization at actin |
| | | | filament/plasma membrane association sites.) 2.9e- |
| | | | 57 |
| ugs024 | MMMHC29N7 | AF030001 | Mus musculus major histocompatibility locus |
| | | | class III re (79848 nt) 2.9e-05 |
| ugs126 | | AB008110 | Rattus norvegicus RTI-DOb gene, partial cds. |
| | | | 7/98 (8818 nt) 4.6e-13 (major histocompatibility |
| | | | gene) |
| ugs133 | MUSTCRA | M64239 | Mouse T-cell receptor alpha/delta chain locus. |
| | | | 8/92 (79772 nt) 1.8e-10 |

Transcription Factors/Co-factor

| | | | |
|---|---|---|---|
| ug141rcon | HSU10323 | U10323 | Human nuclear factor NF45 mRNA, complete |
| | | | cds. 8/94 (1552 nt) 1.9e-32 |
| ug156rcon | | AF075587 | Homo sapiens protein associated with Myc |
| | | | mRNA, complete cds. 8/98 (14807 nt) 8.1e-94 |
| ug157rcon | | AF010403 | Homo sapiens ALR mRNA, complete cds. 9/97 |
| | | | (trx-G paralogue, trithorax gene complex, |
| | | | homeotic) (15789 nt) |
| | | | 8.2e-21 |
| ug159 | MMU92454 | U92454 | Mus musculus WW domain binding protein 5 |
| | | | mRNA, partial cds. (proline-rich, sh3 domain |
| | | | interactive protein) involved in regulation of |
| | | | transcription in development of kidney and limbs. |
| | | | Homologue of Drosophila enabled. (647 nt) |
| ug192rcon | HSU05040 | U05040 | Human FUSE binding protein mRNA, complete |
| | | | cds. 5/94 (2325 nt) 1.4e-69 (The far upstream |
| | | | element-binding proteins comprise an ancient |
| | | | family of single-strand DNA-binding |
| | | | transactivators; myc gene transcriptional |
| | | | controller) |
| ug224 | RRU17837 | U17837 | Rattus sp. zinc finger protein RIZ mRNA, |
| | | | complete |
| | | | cds. 8/95 (6152 nt) 8.7e-29 |
| ug278 | MMU48363 | U48363 | Mus musculus transcriptional activator alpha- |
| | | | NAC (nascent polypeptide-associated complex) |
| | | | gene (12989 nt) 2.3e-20 |
| ug371cons | MMHOXD11 | X71422 | M.musculus Hoxd-11 gene. 8/93 (5593 nt) 1.4e- |
| | | | 63 |
| ug408 | MMU70139 | U70139 | Mus musculus putative CCR4 protein mRNA, |
| | | | partial cds. 7/97 (9737 nt) 1.3e-07 |
| | | | Characterization of two age-induced intracisternal |
| | | | A-particle-related transcripts in the mouse liver. |
| | | | Transcriptional read-through into an open reading |
| | | | frame with similarities to the yeast ccr4 |
| | | | transcription factor. |
| ug422 | | AF098161 | Mus musculus timeless homolog mRNA, |
| | | | complete cds. 11/98 (4438 nt) 7.1e-47 |
| | | | (Mammalian Circadian Autoregulatory Loop: A |
| | | | Timeless Ortholog and mPER1 Interact and |
| | | | Negatively Regulate CLOCK-BMAL1-Induced |
| | | | Transcription) |
| ug442 | | AF017085 | Mus musculus BAP-135 homolog (general |
| | | | transcription factor II-1: Gtf2i: Diws1t) mRNA, |
| | | | complete cds. 3/98 (4091 nt) 2.9e-104 |
| ug509 | | AF059275 | Mus musculus heat shock transcription factor 1 |
| | | | (Hsfl) gene, parti (11395 nt) 4.9e-19 |
| ugs045 | | AF056002 | Rattus norvegicus Smad4 protein (Smad4) mRNA, |
| | | | complete cds. 4/98 (3041 nt) 1.5e-36 |
| ugs055 | RNCEBPRNA | X64403 | R.norvegicus c/ebp (CC-AAT/enhancer binding |
| | | | protein) gamma mRNA. 6/93 (1430 nt) 1.3e-14 |
| ugs107 | HUMYZ84E01 | AF086085 | Homo sapiens full length insert cDNA clone |
| | | | YZ84E01. 8/9 (650 nt) 2.2e-21 similar to chicken |
| | | | SSDP (sequence-specific single-stranded DNA- |
| | | | binding protein), binds pyrimidine rich regions of |
| | | | DNA |
| ugs192 | | AF075587 | Homo sapiens protein associated with Myc |
| | | | mRNA, complete cds. 8/98 (14807 nt) 8.5e-53 |
| ugs210 | RNU09567 | U09567 | Rattus norvegicus cysteine-rich zinc-finger protein |
| | | | mRNA, widely expressed in fetal brain.. (1403 nt) |
| | | | 9.9e-09 |
| ugs213 | MMU41285 | U41285 | Mus musculus dishevelled-3 (Dvl-3) mRNA, |
| | | | complete cds. 6/96 (2498 nt) 3.8e-13 |
| ugs218 | HUMHPLK | M55422 | Human Krueppel-related zinc finger protein (H- |
| | | | plk) mRNA, com (2873 nt) 1.2e-07 |

Nuclear/Mitosis Assoc./Chromatin

| | | | |
|---|---|---|---|
| ug232 | RATHMG2 | D84418 | Rat mRNA for chromosomal protein HMG2, |
| | | | complete cds. 4/97 (1072 nt) 6.7e-51 |
| ug246 | HSCGGBP | AJ000258 | Homo sapiens trinucleotide repeat 5-d(CGG)n- |
| | | | double stranded DNA binding protein (779 nt) |
| | | | 4.2e-21 (Fragile X Assoc) |
| ug248 | MMHMG17 | X12944 | Mouse mRNA for HMG-17 chromosomal protein. |
| | | | 9/93 (1113 nt) 1.6e-69 (HMGs are associated with |
| | | | active chromatin jdr) |
| ug281 | HSU30872 | U30872 | Human mitosin mRNA (mitotic progression |
| | | | factor), complete cds. 12/95 (10211 nt) 8.4e-16 |
| ug340 | MMU39074 | U39074 | Mus musculus thymopoietin beta mRNA, |
| | | | complete cds. 5/96 Ubiquitously expressed |
| | | | nuclear proteins. (2170 nt) 6e-74 |
| ug355 | HSU70322 | U70322 | Human transportin (TRN) mRNA, Alternative |
| | | | mechanism to NTS for nuclear translocation. A |
| | | | receptor mediated mechanism via transportin. |
| | | | 10/96 (3054 nt) 2.7e-11 |
| ug453 | | AF033664 | Mus musculus gene-trap line CT 146 cbp 146 |
| | | | (cbp 146) mRNA, Capturing novel mouse genes |
| | | | encoding chromosomal and other nuclear |
| | | | proteins(1032 nt) 6e-100 |
| ug487 | MMPSHIS2B | X90779 | M.musculus psH2B gene. 3/97 (1312 nt) 1.3e-38 |
| | | | (Molecular cloning of mouse somatic and testis- |
| | | | specific H2B histone genes containing a |
| | | | methylated CpG island.) |
| ugs026 | MMAJ2636 | AJ002636 | Mus musculus mRNA for nuclear protein SA2. |
| | | | 11/97 (3871 nt) 4.9e-34 |
| ugs090 | | AB015330 | Homo sapiens mRNA for HRIHFB2007, |
| | | | Selection system for genes encoding nuclear- |
| | | | targeted proteins. 12/98 (865 nt) 1.7e-26 |
| ugs174 | | AF083322 | Homo sapiens centriole associated protein |
| | | | CEP110 mRNA, complete c (3893 nt) 2.3e-29 |
| ugs205 | RATSP120 | D14048 | Rat mRNA for SP120,Nuclear scaffold protein |
| | | | that binds the matrix attachment region DNA 1/95 |
| | | | (3563 nt) 3e-28 |
| ugs227 | MMU18295 | U18295 | Mus musculus histone Hi (0) gene, complete cds. |
| | | | 7/95 (2893 nt) 5.2e-42 (An upstream control |
| | | | region required for inducible transcription of the |
| | | | mouse H1(zero) histone gene during terminal |
| | | | differentiation.) |
| ugs232 | HSNUMAMRB | Z11584 | H.sapiens mRNA for NuMA protein. 4/92 |
| | | | (mitotic spindle associated protein)(7217 nt) 2.8e-32 |
| ugs234 | | AF022465 | Mus musculus high mobility group protein |
| | | | homolog HMG4 (Hmg4) mRNA (1502 nt) 3.9e- |
| | | | 44 (The mouse Hmg4 gene is highly expressed in |
| | | | the embryo; Hmg4 transcripts are barely |
| | | | detectable in adult tissues. The human HMG4 |
| | | | gene, which is extremely similar to its mouse |
| | | | homolog, has been sequenced as part of |
| | | | chromosome X, band q28. HMG4, HMG1, and |
| | | | HMG2 proteins have been highly conserved |
| | | | during vertebrate evolution, suggesting that each |
| | | | has at least some unique property. It is possible |
| | | | that HMG4 is required during development) |

Mitochondrial

| | | | |
|---|---|---|---|
| ug104rcon | RATMT3H3MG | M63800 | Rattus norvegicus mitochondrial 3-hydroxy-3- |
| | | | methyl glutaryl coenzyme alpha-synthase gene, |
| | | | exon 1 (2074 nt) 4.5e-08 |
| ug180rcon | MUSMTHYPB | L07096 | Mus domesticus strain Mi1P mitocondrion |
| | | | genome, complete s (16303 nt) 1.5e-11 |
| ug181rcon | MUSMTHYPA | L07095 | Mus domesticus strain NZB/B1NJ mitochondrion |
| | | | genome, compl (16303 nt) 5.3e-37 |
| ug205 | MUSMTHYPB | L07096 | Mus domesticus strain Mi1P mitochondrion |
| | | | genome, complete s (16303 nt) 1.1e-51 |
| ug220 | MUSMTHYPA | L07095 | Mus domesticus strain NZB/B1NJ mitochondrion |
| | | | genome, compl (16303 nt) 9.4e-110 |
| ug240 | MUSMTHYPA | L07095 | Mus domesticus strain NZB/B1NJ mitochondrion |
| | | | genome, compl (16303 nt) 6.7e-60 |
| ug411 | MUSMTHYPA | L07095 | Mus domesticus strain NZB/B1NJ mitochondrion |
| | | | genome, compl (16303 nt) 8.2e-87 |
| ug448 | MUSMTHYPA | L07095 | Mus domesticus strain NZB/B1NJ mitochondrion |
| | | | genome, compl (16303 nt) 2.6e-88 |
| ug499 | MUSMTCG | J01420 | Mouse mitochondrion, complete genome. 7/95 |
| | | | (16295 nt) 1e 19 |
| ugs104 | MUSMTHYPA | L07095 | Mus domesticus strain NZB/B1NJ mitochondrion |
| | | | genome, compl (16303 nt) 2.9e-56 |
| ugs178 | MUSMTHYPB | L07096 | Mus domesticus strain MilP mitocondrion |
| | | | genome, complete s (16303 nt) 1.2e-53 |

RNA Splicing, Binding, RNPs, etc...

| | | | |
|---|---|---|---|
| ug185 | MUSCIRPB | D78135 | Mus musculus mRNA for CIRP, complete cds. |
| | | | 2/98 (1256 nt) 8.2e-16 (CIRP = cold-inducible RNA-binding |
| | | | protein jdr) |
| ug304 | HSU97188 | U97188 | Homo sapiens putative RNA binding protein KOC |
| | | | (koc) mRNA, c (4181 nt) 8.3e-31 |
| *ug485 | MUSCIRPB | D78135 | Mus musculus mRNA for CIRP, complete cds. |
| | | | 2/98 (1256 nt) 7e-09 |
| *ug494cons | HUMASF | M72709 | Human alternative splicing factor mRNA, |
| | | | complete cds. 9/91 (1717 nt) 1.2e-27 |
| ugs060 | HSU85510 | U85510 | Human RNA polymerase II subunit hsRPB4 |
| | | | mRNA, complete cds, (1894 nt) 1.9e-28 |
| ugs102 | HSPABII | Y08772 | H.sapiens PABII pseudogene, poly(A) binding |
| | | | protein. 1/97 (1930 nt) 8.8e-18 |
| ugs106 | MMU40654 | U40654 | Mus musculus U22 snoRNA host gene (UHG) |
| | | | gene, complete sequ (3838 nt) 2.6e-22 (These |
| | | | snoRNAs are co-transcribed with their host pre- |
| | | | mRNAs and released by processing from excised |
| | | | introns. Here we show that, in addition to U22, |
| | | | seven novel fibrillarin-associated snoRNAs, |
| | | | named U25-U31, are encoded within different |
| | | | introns of the unusually compact mammalian U22 |
| | | | host gene (UHG). All seven RNAs exhibit |
| | | | extensive (12-15 nucleotides) complementarity to |
| | | | different segments of the mature rRNAs, |
| | | | followed by a C/AUGA (′U-turn′) sequence. The |
| | | | spliced UHG RNA, although it is associated with |
| | | | polysomes, has little potential for protein coding, |
| | | | is short-lived, and is poorly conserved between |
| | | | human and mouse. Thus, the introns rather than |
| | | | the exons specify the functional products of |
| | | | UHG.) |
| ugs159 | HUMU1RNP1 | M60779 | Human U1 snRNP-specific protein A gene, exon |
| | | | 1.1/95 (495 nt) 2.6e-14 |

Peptidases, Proteinases, Isomerases,
Transferases

| | | | |
|---|---|---|---|
| ug088rcon | MM14MMP9 | AF022432 | Mus musculus matrix metalloproteinase-14 |
| | | | (Mmp14), exons 9 (1242 nt) 2e-38 |
| ug179rcon | | AF090430 | Mus musculus ATP-dependent metalloprotease |
| | | | FtsH1 mRNA, complete c (2654 nt) 9.5e-15 |
| ug426 | MAP5PRQMR | X62678 | M.auratus mRNA for P5 protein. 8/93 (2234 nt) a |
| | | | member of the protein disulphide isomerase/form I |
| | | | phosphoinositide-specific phospholipase C family |
| | | | 3.2e-21 |

Developmental Unclassified

| | | | |
|---|---|---|---|
| ug109rcon | RATDRP | L20319 | Rattus norvegicus developmentally regulated |
| | | | protein mRNA, com (5395 nt) 3e-60 |
| ug380 | MUSMEAA | L10401 | Mus musculus male-enhanced antigen (Mea) |
| | | | mRNA (human chromo 6p21.1-21.3), complete |
| | | | cds. (841 nt) 1.1e-38 |
| ug423 | | AF015262 | Homo sapiens Down Syndrome critical region, |
| | | | partial sequence. 2/9 (79607 nt) 1.9e-10 |
| ugs008 | MMU47024 | U47024 | Mus musculus maternal-embryonic 3 (Mem3) |
| | | | mRNA, complete cds (3128 nt) 8.8e-12 |

Protein Turnover

| | | | |
|---|---|---|---|
| ug234 | | AF071317 | Mus musculus COP9 complex subunit 7b |
| | | | (COPS7b) mRNA, complete cds. (1990 nt) 2.2e- |
| | | | 91 (The COP9 complex is conserved between |
| | | | plants and mammals and is related to the 26S |
| | | | proteasome regulatory complexsubunit. 7b is a |
| | | | component of the COP9 complex which contains a |
| | | | total of 8 distinct subunits, similar tothe JAB 1- |
| | | | containing signalosome; the plant COP9 complex |
| | | | functions as a repressor of photomorphogenesis) |
| ug267 | MMU96635 | U96635 | Mus musculus ubiquitin protein ligase Nedd-4 |
| | | | mRNA, complete (5581 nt) 6.7e-50 |
| ug445 | | AF033353 | Mus musculus ubiquitin-homology domain protein |
| | | | (Ubl1) mRNA, compl (1187 nt) 2.1e-87 |

X Chromosome Associated

| | | | |
|---|---|---|---|
| ug115rcn1o | HS23K20 | AL022153 | Human DNA sequence from clone 23K20 on |
| | | | chromosome Xq25-26. (79472 nt) 4.9e-07 |
| ug196 | | AC004827 | Homo sapiens PAC clone DJ044L15 from Xq23, |
| | | | complete sequence. 10/(79688 nt) 2.5e-25 |
| ug321 | MMTSXDNA | X99946 | M.musculus 94kb genomic sequence encoding |
| | | | Tsx (testis-specific X-chromosome) gene. 11/96 |
| | | | (79555 nt) 2.1e-09 |
| ug328 | | AB006651 | Homo sapiens EXLM1 mRNA, complete cds. |
| | | | 6/98 (7984 nt) 1.1e-116 (Detection and isolation |
| | | | of a novel human gene located on Xp11.2-p11.4 |
| | | | that escapes X-inactivation) |
| ug385 | HSA218J18 | AL034370 | Human DNA sequence from clone 218J18 on |
| | | | chromosome Xp11. (40465 nt) 1e-19 |
| ug390 | HSA218J18 | AL034370 | Human DNA sequence from clone 218J18 on |
| | | | chromosome Xp11. (40478 nt) 9.5e-19 |
| *ugs194rs | | AC005859 | Homo sapiens Xp22-83 BAC GSHB-324M7 |
| | | | (Genome Systems Human BAC Lib (79502 nt) |
| | | | 3.2e-07 |

Chromosomal Locus Association

| | | | |
|---|---|---|---|
| ug078rcon | | AC004132 | Homo sapiens chromosome 17, clone |
| | | | hRPC.986_F_12, complete sequence (79432 nt) |
| | | | 2.1e-12 |
| ug089rcon | HSAC001228 | AC001228 | 244Kb Contig from Human Chromsome 11p15.5 |
| | | | spanning D11S (79627 nt) 2.4e-21 |
| ug134 | HS66H14 | Z97989 | Human DNA sequence from PAC 66H14 on |
| | | | chromosome 6q21-22. Con (76686 nt) 8.9e-26 |
| ug210c | HS82J11 | Z83850 | Human DNA sequence from PAC 82J11 and |
| | | | cosmid U134E6 on chrom (79596 nt) 3.4e-14 |
| ug286 | AC004611 | | Homo sapiens chromosome 19, cosmid F24200, |
| | | | complete sequence. 4/9 (47055 nt) 2.7e-20 |
| ug323 | HS180M12 | Z82190 | Human DNA sequence from PAC 180M12 on |
| | | | chromosome 22. Contains GSSs (59941 nt) 3e-16 |
| ug346 | | AC002121 | Genomic sequence from Mouse 11, complete |
| | | | sequence. 7/97(79740 nt) 2.2e-11 (poly A jdr) |
| ug350 | | AC002324 | Mus musculus chromosome 11, clone 475_H_14, |
| | | | complete sequence. 5/ (79709 nt) 2.1 e-26 |
| ug364 | HUAC002550 | AC002550 | Human Chromosome 16 BAC clone CIT987SK- |
| | | | A-101F10, comple (79780 nt) 5.9e-15 (poly Ajdr) |
| ug370 | HS434P1 | Z97056 | Human DNA sequence from PAC 434P1 on |
| | | | chromosome 22. Contains (45764 nt) 4.7e-24 |
| ug395 | HSL241B9C | Z69708 | Human DNA sequence from cosmid L241B9, |
| | | | Huntington's Diseas (17243 nt) 1.7e-21 |
| ug402 | | AP000031 | Homo sapiens genomic DNA, chromosome |
| | | | 21q11.1, segment 2/28, compl (79580 nt) 2.6e-10 |
| ug407 | | AC002116 | Human DNA from chromosome 19 cosmid |
| | | | R33743, genomic sequence, com (40491 nt) 4.1e-09 |
| ug450 | | AC000399 | Genomic sequence from Mouse 9, complete |
| | | | sequence. 5/97 (61336 nt) 1.2e-08 |
| ug457 | | AC003063 | Mus musculus Chromosome 16 BAC Clone b40- |
| | | | o20 Syntenic To Homo sap (79720 nt) 2.1 e-11 |
| ug461 | | AC005807 | Mus musculus chromosome 17 BAC clone |
| | | | citb585c7 from MHC region, c (65870 nt) 4.7e-07 |
| ug467 | HSU96629 | U96629 | Human chromosome 8 BAC clone CIT987SK- |
| | | | 2A8 complete sequence (79589 nt) 7.2e-08 |
| ug470 | | AC003018 | Mus musculus Chromosome 4 BAC84c8, |
| | | | complete sequence. 5/98 (57327 nt) 4.8e-10 |
| ug476 | | AC002324 | Mus musculus chromosome 11, clone 475_H_14, |
| | | | complete sequence. 5/ (79604 nt) 1.5e-16 |
| ug477 | | AC005900 | Homo sapiens chromosome 17, clone |
| | | | hRPK.998_F_8, complete sequence (79544 nt) |
| | | | 1.3e-07 |
| ug488 | HS459L4 | AL031120 | Human DNA sequence from clone 459L4 on |
| | | | chromosome 6p22.3-2 (79692 nt) 4.2e-08 |
| ug497 | | AC002324 | Mus musculus chromosome 11, clone 475_H_14 |
| | | | complete sequence. 5/ (79672 nt) 6.8e-11 |
| ug524 | MMNHCHMG1 | Z11997 | M.musculus mRNA for non-histone chromosomal |
| | | | high-mobility (2231 nt) 2.3e-69 |
| ugs017 | | AC004790 | Homo sapiens chromosome 19, cosmid F17987, |
| | | | complete sequence. 6/9 (41613 nt) 4.4e-20 |
| ugs021 | HSD13S106 | X59131 | Homo sapiens D13S106 mRNA for A unique |
| | | | intronless gene or gene seqment on chromosome |
| | | | 13 specifying a highly charged amino acid |
| | | | sequence (3650 nt) 1.5e-12 |
| ugs033 | | AC005259 | Mouse BAC CitbCJ7 219m7, genomic sequence, |
| | | | complete sequence. 7/9 (79776 nt) 5e-09 |
| ugs036 | | AC005742 | Mus musculus chromosome 11, BAC clone 111- |
| | | | 181 (LBNL M01), complet (79780 nt) 7.3e-07 |
| ugs116 | | AC004500 | Homo sapiens chromosome 5, P1 clone 1076B9 |
| | | | (LBNL H14), complete s (77538 nt) 1.3e-26 |
| ugs125 | | AC004259 | Human Chromosome 15q11-q13 PAC clone |
| | | | pDJ14i12 containing Angelman (79744 nt) 2.3e-07 |
| ugs134 | | AF059580 | Murine genomic DNA; partially digested Sau3A |
| | | | fragment, cloned int (36326 nt) 3e-45 |
| ugs139 | HS94G16 | Z85999 | Human DNA sequence from PAC 94G16 on |
| | | | chromosome 6q21. Contai (79812 nt) 5.6e-07 |
| ugs165 | | AP000021 | Homo sapiens genomic DNA, chromosome |
| | | | 21q22.2 (Down Syndrome regio (79776 nt) 3.1e-08 |
| ugs191 | | AC005070 | Homo sapiens BAC clone RG152G17 from 7q22- |
| | | | q31.1, complete sequenc (79788 nt) 4.3e-16 |
| ugs208 | | AF044773 | Homo sapiens breakpoint cluster region protein in |
| | | | uterine leiosarcoma (chromo t12:14) (BCRG1) |
| | | | mRNA, co (772 nt) 2e-12 |
| ugs219 | DJ270M14 | AF107885 | Home sapiens chromosome 14q24.3 clone |
| | | | BAC270M14 transform (79780 nt) 2.3e-11 |

Heat Shock, Chaperones, Protein Trafficking

| | | | |
|---|---|---|---|
| ug147 | HUMCALIEF | M94859 | Human calnexin mRNA (molecular chaperone), |
| | | | complete cds. 9/94 (3881 nt) 7.8e-22 |
| ug245 | RNHSP7O3 | X77209 | R.norvegicus Hsp70-3 gene. 1/97 (3913 nt) 7.8e-25 |
| ug348 | MMSCP2EX6 | X91155 | M.musculus scp2 gene exon 6. 1/97 (512 nt) 1.2e-21 |
| | | | (the murine sterol carrier protein 2 gene |
| | | | (Scp2)) |
| ug400 | MMHSP47 | X60676 | M.musculus HSP47 mRNA. 6/93 (2273 nt) 4.9e- |
| | | | 104 |
| ug435 | | AF058718 | Homo sapiens putative 13 S Golgi transport |
| | | | complex 90kD subunit brain-specific isoform |
| | | | mRNA, complete cds. (3105 nt) 2.6e-07 |
| ug455 | HSU67615 | U67615 | Human beige protein homolog (chs) mRNA, |
| | | | complete cds. 1/97 (13449 nt) 3.4e-11 (beige |
| | | | gene is involved in protein and lysosomal |
| | | | trafficking) |
| ug507 | RATNOP140A | M94287 | Rattus norvegicus nucleolar phosphoprotein of |
| | | | 140kD, Nopp (3609 nt) 1.7e-08 Molecular |
| | | | chaperone for NTS containing proteins. |
| ugs019 | HUMHBP | M64098 | Human high density lipoprotein binding protein |
| | | | (HBP) mRNA, co (4354 nt) 8.7e-36 |

Neural Element or Assoc.

| | | | |
|---|---|---|---|
| ug198 | | AF047384 | Rattus norvegicus postsynaptic protein CRIPT |
| | | | mRNA, complete cds. (1435 nt) 2.7e-23 |
| ug261 | HSGTHLA1 | Y11044 | Homo sapiens mRNA for GABA-BR1a (hGB1a) |
| | | | receptor. 10/98 (4220 nt) 3.8e-12 |
| ug333 | MUSSPESPEP | M55181 | Mouse spermatogenic-specific proenkephalin |
| | | | mRNA, complete (1408 nt) 3.2e-57 |

DNA Repair

| | | |
|---|---|---|
| ug099rcon | AF069519 | Mus musculus T:G mismatch-specific thymine- |
| | | DNA glycosylase TDGb i(2859 nt) 1.1e-51 |

Metabolism (Cytosolic)

| | | | |
|---|---|---|---|
| ug125 | RNO010709 | AJ010709 | Rattus norvegicus gene encoding tyrosine |
| | | | aminotransferase (12460 nt) 5.3e-16 |
| ug266 | MUSCATALAA | L25069 | Mouse catalase mRNA (antioxidant enzyme), |
| | | | complete cds. 5/95 (2423 nt) 1.4e-54 |
| ug366 | HSU62961 | U62961 | Human succinyl CoA: 3-oxoacid CoA transferase |
| | | | precursor (OXC (3337 nt) 3.4e-11 |
| ugs022 | MSALEN | X52379 | Mouse mRNA for alpha-enolase (2-phospho-D- |
| | | | glycerate hydrolase (1720 nt) 2.2e-46 |

Unknown

| | | | |
|---|---|---|---|
| *ug317 | MMU80894 | U80894 | Mus musculus CAG trinucleotide repeat mRNA, |
| | | | Transcription factor or Cadherin. (543 nt) 6.8e-51 |
| ugs025 | | AF052130 | Homo sapiens clone 23704 mRNA sequence. 8/98 |
| | | | (1810 nt) 1.7e-09 |
| ugs080 | MUSHKPRO | M74555 | Mouse house-keeping protein mRNA, complete |
| | | | cds. 8/91 (2415 nt) 2.7e-54 |
| *ugs506or | MMY17106 | Y17106 | Mus musculus transposon ETn, SELH/L3A strain. |
| | | | 10/98 (5542 nt) 2.5e-73 |
| ugs042 | MMY17106 | Y17106 | Mus musculus transposon ETn, SELH/L3A strain. |
| | | | 10/98 (5542 nt) 5.1e-50 |
| ug160 | | AB014563 | Homo sapiens mRNA for KIAA0663 protein, |
| | | | complete cds. 7/98 (4365 nt) 3.2e-60 |
| ug178rcon | | AB011125 | Homo sapiens mRNA for KIAA0553 protein, |
| | | | partial cds. 4/98 (5574 nt) 2.9e-26 |
| ug209 | | D86971 | Human mRNA for KIAA0217 gene, partial cds. |
| | | | 7/97 (5404nt) 2.4e-42 |
| ug213 | | D86971 | Human mRNA for KIAA0217 gene, partial cds. |
| | | | 7/97 (5404 nt)7.3e-41 |
| ug263 | | AB014550 | Homo sapiens mRNA for KIAA0650 protein, |
| | | | partial cds. 7/98 (5003 nt) 2.4e-79 |
| ug275 | HUMORF16 | D14812 | Human mRNA for KIAA0026 gene, complete |
| | | | cds. 7/97 (1826 nt) 3.2e-45 |
| ug377 | HUMORF16 | D14812 | Human mRNA for KIAA0026 gene, complete |
| | | | cds. 7/97 (1826 nt) 1.5e-66 |
| ug481cp2 | | AB018325 | Homo sapiens mRNA for KIAA0782 protein, |
| | | | partial cds. 11/98 (4130 nt) 5.2e-10 |
| ugs027 | | AB018272 | Homo sapiens mRNA for KIAA0729 protein, |
| | | | partial cds. 11/98 (4143 nt) 4.2e-51 |
| ugs029 | | AB018306 | Homo sapiens mRNA for KIAA0763 protein, |
| | | | complete cds. 11/98 (4148 nt) 1.1e-27 |
| ugs099 | | AB002293 | Human mRNA for K1AA0295 gene, partial cds. |
| | | | 6/97 (7326nt) 6.4e-40 |
| ugs100 | | D86958 | Human mRNA for KIAA0203 gene, complete |
| | | | cds. 7/97 (6614nt) 8.5e-40 |
| ugs211 | | AB018325 | Homo sapiens mRNA for KIAA0782 protein, |
| | | | partial cds. 11/98 (4130 nt) 1.2e-19 |
| ugs235 | | AB018330 | Homo sapiens mRNA for KIAA0787 protein, |
| | | | partial cds. |
| | | | 11/98 (4427 nt) 2.2e-13 |

Table 4 presents the results of the library analysis of 787 cDNA UGS-derived ESTs using the GenBank database.

TABLE 4
Results of the library analysis of 787 cDNA UGS-derived ESTs
using the GenBank database

| Clone | Locus | Acc. # | Identity |
|---|---|---|---|
| | | | GTPases |
| ua1b5 | RNARP1 | X78603 | R.norvegicusSprague DawleyARP1 mRNA faD |
| | | | ARF-related prote (943 nt) 1.3e-19 (ARP is a |
| | | | plasma membrane-associateD Ras-related GTPase |
| | | | with remote similarity to the family oD ADP |
| | | | ribosylation factors.D |
| ua1e1r | HSU18420 | U18420 | Human ras-related small GTP binding protein |
| | | | Rab5rab5mRN (1590 nt) 4.8e-27 |
| ug035con | HSPACAP | X60435 | H.sapiens gene PACAP for pituitary adenylate |
| | | | cyclase activating polypeptide (PACAP) (17041 |
| | | | nt) 9.5e-08 |
| ug182 | RATGCA | J05677 | Rat guanylyl cyclase A/atrial natriuretic peptide |
| | | | receptor G (17517 nt) 9.9e-07 |
| | | | Protein Kinases/Phosphatases |
| ug069rcon | HSPTP1CHG | X82818 | H. sapiens PTP1C/HCP gene.protein tyrosine |
| | | | phosphatase. June 1997 (8545 nt) 2.7e-30 |
| | | | Structural Proteins/ECM |
| ua1e3f | MMU49739 | U49739 | Mus musculus unconventional myosin |
| | | | VIsvmRNAD complete c (4602 nt) 2e-36 |
| ug013rcon | RATCRBGLVC | L20468 | Rattus norvegicus cerebroglycan mRNAD |
| | | | complete cds. January 1994 (2607 nt) 2e-23 |
| ug031con | AF078705 | Z97056 | Mus musculus vascular adhesion protein-1 gene. |
| | | | complete cds. September 1998 (14357 nt) 6.5e-12 |
| ug055con | MMLAMBETA2 | U43541 | Mus musculus laninin beta 2 gene, exon 17-33, |
| | | | (5350 nt) 2.3e-95 |
| ug059 | AB009808 | AF085906 | Homo sapiens gene for osteonidogen, intron 9. |
| | | | March 1998 (9085 at) 2.2e-08 |
| ug464 | MMSYNDE1A | Z22532 | M. musculus syndecan-1. April 1997 (33934 nt) 6.7e-07 |
| | | | Oncogenes/Tumor Suppressors/Apoptosis |
| ug039rcon | MMAF000168 | AF000168 | Mus musculus 9ORF binding protein 19BP-1 |
| | | | mRNA, Binding of Human Virus Oncoproteins to |
| | | | bD1g/SAP97, a Mammalian Homolog of the |
| | | | Drosophila Discs large Tumor Suppressor protein |
| | | | (2703 nt) 2.1e-155 |
| | | | Growth Factors, Cytokines & Binding Proteins |
| ua1a4f | AF063020 | Z11584 | Homo sapiens Lens epithelium-derived growth |
| | | | factor (LEDGF) mRNA (3377 nt) 1.5e-24 |
| ua1e6r | MMU7909 | AJ007909 | Mus musculus mRNA for erythroid differentiation |
| | | | regulator, A novel protein from WEHI-3 cells |
| | | | inducing hemoglobin synthesis in human K562 |
| | | | and murine erythroleukemia cells (715 nt) 3.8e- |
| | | | 16 |
| ua1f5f | AF063020 | AJ007909 | Homo sapiens lens epithelium-derived growth |
| | | | factor mRNA, A novel protein from WEHI-3 cells |
| | | | inducing hemoglobin synthesis in human K562 |
| | | | and murine erythroleukemia cells complete (715 |
| | | | nt) 5.5e-23 |
| ua1g2f | MMU7909 | AJ007909 | Mus musculus mRNA for erythroid differentiation |
| | | | regulator A novel protein from WEHI-3 cells |
| | | | inducing hemoglobin synthesis in human K562 |
| | | | and murine erythroleukemia cells, (715 nt) 2.4e-26 |
| ua2h6r | MUSIGFBP04 | L05439 | Mouse insulin-like growth factor binding protein 2 |
| | | | (IGFBP-2) (532 nt) 3.4e-73 |
| ug051rcon | MMTHYMOA | X56135 | Mouse mRNA for prothymosin alpha. June 1991 (1191 |
| | | | nt) 8.7e-56 |
| | | | Ribosomal Proteins and rRNA |
| ug037rcon | MM45SRRNA | X82564 | M. musculus 45S pre rRNA gene. April 1996 (22118) |
| | | | nt4.6e-99 |
| | | | Transcription Factors/Co-factors |
| ug011rcon | MMCNBPMR | X63866 | Mus musculus mRNA for cellular nucleic acid |
| | | | binding protein (1492 nt) 1.5e-102 |
| ug033con | MMTSC22 | X62940 | M. musculus TSC-22 mRNA. Isolation of a gene |
| | | | encoding a putative leucine zipper structure that is |
| | | | induced by transforming growth factor beta 1 and |
| | | | other growth factors. December 1993 (1706 nt) 6e-128 |
| ug053rcon | D87671 | X56135 | Rat mRNA for TIP120, TATA-binding protein |
| | | | interacting protein. January 1997 (4383 nt) 7.8e-84 |
| *ug092 | GGU68380 | U68380 | Gallus gallus single-strand DNA-binding protein. |
| | | | csdp SSDP |
| | | | (sequence-specific single-stranded DNA-binding |
| | | | protein), mRNA,(1211 nt) 5.2e-85 |
| ugs045 | AF056002 | Z22532 | Rattus norvegicus Smad4 protein Smad4 mRNA, |
| | | | complete cds. April 1998 (3041 nt) 8.3e-36 |
| | | | Mitochondrial |
| ug292 | AA933159 | D21852 | UI-R-E0-cz-e-07-0-UI.s1 UI-R-E0 Rattus |
| | | | norvegicus cDNA clone UI-R (283 nt) 1.9e-33 |
| | | | (Rat mitochondrial genome fragment encoding |
| | | | cytochrome oxidase subunit I) |
| ugs104 | MUSMTHYPB | L07096 | Mus domesticus strain MilP mitocondrion |
| | | | genome, complete seq (16303 nt) 6.3e-48 |
| | | | RNA Splicing, Binding, RNPs, etc . . . |
| ug034con | AF015812 | X62940 | Homo sapiens RNA helicase p68HUMP68gene, |
| | | | complete cds. November 1997 (7834 nt) 4.5e-111 |
| | | | Peptidases, Proteinases, Isomerases, |
| | | | Transferases |
| ug238 | HUMCANPRA | J04700 | Homo sapiens calcium-dependent protease large |
| | | | subunit CAN (1154 at) 8.4e-07 |
| ug406 | RNU05013 | U05013 | Rattus norvegicus Sprague-Dawley heme |
| | | | oxygenase-2 non-reducing form, genomic clone |
| | | | (14984 nt) 1.5e-08 |
| | | | Developmental Unclassified |
| ug251 | AU043179 | AU043179 | Mouse sixteen-cell-embryo cDNA Mus musculus |
| | | | cDNA clone J (550 nt) 3.8e-75 |
| ug373 | AI550071 | AU051101 | mn04d09.y1 Beddington mouse embryonic region |
| | | | Mus musculus cDNA clone (508 nt) 3.3e-65 |
| ugs060 | AI331913 | Z22532 | fa95b11.y1 zebrafish fin day3 regeneration Danio |
| | | | rena cDNA 5′si (476 nt) 5e-34 |
| | | | Protein Turnover |
| ug066rcon | MMUBIQU | X51703 | Mouse mRNA for ubiquitin. May 1991 (1172 nt) 2.8e- |
| | | | 30 |
| | | | X Chromosome Associated |
| ug158 | AF002223 | L27758 | Human genomic DNA of Xq28 with MTM1 and |
| | | | MTMR1 genes, complete seq (79535 nt) 2.8e-13 |
| | | | (MTM1 gene mutations in 47 unrelated X-linked |
| | | | myotubular myopathy patients.) |
| | | | Chromosomal Locus Associated |
| ua1a6r | AC004453 | Z11584 | Homo sapiens PAC clone DJ0844F09 from 7p12- |
| | | | p13D complete sequence (79660 nt) 1e-10 |
| ug021rcon | AC003997 | L20468 | Mouse BAC mbac20 from 14D1-D2T-Cell |
| | | | Receptor AlphD Locus, (79656 nt) 8.1e-56 |
| ug028rcon | HS434P1 | Z97056 | Human DNA sequence from PAC 434P1 oD |
| | | | chromosome 22. Contains (45869 nt) 3.6e-13 |
| ug036rcon | AC004079 | X62940 | Homo sapiens PAC clone DJ0167F23 from 7p15, |
| | | | completD sequence. (23938) nt 7.1e-63 |
| ug048 | HS434P1 | Z97056 | Human DNA sequence from PAC 434P1 on |
| | | | chromosome 22. Contains (45548 nt) 2.1 e-46 |
| ug050rcon | AC005742 | Z97056 | Mus musculus chromosome 11, BAC clone 111- |
| | | | 181LBND M01, complete (79416 nt) 1.3e-10 |
| ug291ft | B49438 | D21852 | RPCI1 1-6118.TV RPCI11 Homo sapiens genomic |
| | | | clone R-6118, genomic (539 nt) 1.9e-08 |
| ug397 | HS73M5 | AJ010597 | Homo sapiens chromosome 21 PAC |
| | | | RPCIP704M573Q2. March 1999 (79664 nt) 7.8e-08 |
| ug429 | AC005743 | U05013 | Mus musculus PAC clone, complete sequence. |
| | | | December 1998 (185548 nt) 9.3e-13 |
| ug472 | AQ194542 | Z22532 | RPCI11-60K21.TJ RPCI11 Homo sapiens |
| | | | genomic clone R-60K21, genomic clone (388 nt) |
| | | | 9.1e-09 |
| ugs007 | AQ111639 | Z22532 | CIT-HSP-2378O4.TF CIT-HSP Homo sapiens |
| | | | genomic clone 2378O4, geno (634 nt) 5.8e-09 |
| ugs018 | AU026520 | Z22532 | Rattus norvegicus, OTSUKA clone, |
| | | | OT83.06/945f02, microsatellite seq. (298 nt) 3.4e- |
| | | | 08 |
| ugs031 | AC005259 | Z22532 | Mouse BAC CitbCJ7 219m7, genomic sequence, |
| | | | complete sequence. July 1997 (9836 nt) 1.5e-07 |
| ugs032 | AQ240341 | Z22532 | CIT-HSP-2386E2.TF.1 CIT-HSP Homo sapiens |
| | | | genomic clone 2386E2, (576 nt) 8.7e-28 |
| ugs043 | AC004406 | Z22532 | Mus musculus ma40a113, complete sequence. T- |
| | | | cell receptor locus. March 98 (47536 nt) 1.9e-09 |
| ugs065 | HUAC004531 | AC004531 | Home sapiens Chromosome 16 BAC cloneD |
| | | | CIT987SK-A-67A1, clone (79783 nt) 3.5e-10 |
| ugs113 | AC006087 | L07096 | ***SEQUENCING IN PROGRESS***Homo |
| | | | sapiens chromosome 12p13.3 clone (79790 nt) |
| | | | 1.5e-15 |
| ugs127 | AC002327 | AU051628 | Mus musculus chromosome 7, clone 19K5, |
| | | | complete sequence. February 1999 (79756 nt) 1.7e-07 |
| ugs138 | MMU58105 | U58105 | Mus musculus Btk locus, alpha-D-galactosidase |
| | | | AAgs, ribosomal protein (L44L), and Bruton's |
| | | | tyrosine kinase (Btk) genes (79780 nt) 4.8e-07 |
| ugs152 | AC007110 | U58105 | Homo sapiens chromosome 17, clone |
| | | | hRPK.472_J_18, complete sequence (79816) nt |
| | | | 1.1e-07 |
| ugs157 | AC002109 | U58105 | Genomic sequence from Mouse 9, complete |
| | | | sequence. September 97 (79776 nt) 1.1e-07 |
| ugs225 | AC003949 | L27758 | Mus musculus chromosome 19, clone D19-96, B7, |
| | | | complete sequence. (769037 nt) 8.5e-09 |
| ugs228 | AP000025 | L27758 | Homo sapiens genomic DNA, chromosome |
| | | | 21q1 1.1, segment 3/5, complete (31709 nt) 5.8e- |
| | | | 23 |
| | | | Heat Shock, Chaperones, Protein Trafficking |
| ugs203 | AF086628 | L27758 | Homo sapiens Vesicle associated membrane |
| | | | protein (VAMP)-associated protein BVAP-B |
| | | | mRNA, complete cds (2195 nt)1.7e-15 |
| | | | Testis/Sperm or Male enhanced |
| ua2h7r | D78270 | X60435 | Mouse mRNA for male-enhanced antigen-2, |
| | | | complete cds. April 1997 (4621 nt) 1.4e-07 |
| ug058rcon | MMTSXDNA | X99946 | M. musculus 94kb genomic sequence encoding |
| | | | TsD gene. November 1996 (79563 nt) 2.7e-12 (a new testis |
| | | | specific gene TsxD) |
| ug197 | AQ212110 | J05677 | HS_3241_B1_E05_MR CIT Approved Human |
| | | | Genomic Sperm Library Homo sapiens (395 nt) |
| | | | 6.9e-09 |
| ugs078 | AQ303203 | AC004531 | HS_3235_B2_H09_T7 CIT Approved Human |
| | | | Genomic SpermD Library Homo (504 nt) 5.1 e-37 |
| ugs195 | AQ270425 | AL034550 | HS_2052_B1_H06_T7 CIT Approved Human |
| | | | Genomic Sperm Library D Homo (380 nt) 2.6e-23 |
| | | | Metabolism (Cytosolic) |
| ugs080 | MUSHKPRO | M74555 | Mouse house-keeping protein mRNA, complete |
| | | | cds. August 1991 (2415 nt) 3.7e-57 |
| | | | Normalized Library ESTs (Non-Human) |
| ug073rcon | HUMYQ60A05 | AF085906 | Homo sapiens full length insert cDNA clone |
| | | | YQ6OA05. August 1998 (497 nt) 5.3e-15 |
| ug478 | AI384054 | Z22532 | te36a06.x1 Soares_NhHMPu_S1 Homo sapiens |
| | | | cDNA clone IMAGE:2088754 (488 nt) 2.4e-40 |
| ug483 | AI269337 | Z22532 | qj69d02.x1 NCI_CGAP_Kid3 Homo sapiens |
| | | | cDNA clone IMAGE:1864707 3′(416 nt) 3.3e-23 |
| ugs145 | AA724439 | U58105 | ah91h04.s1 Soares_NFL_T_GBC_S1 Homo |
| | | | sapiens cDNA clone IMAGE: 1326 (428 nt) 4.4e- |
| | | | 20 |
| ugs181 | HS1184F4 | AL034550 | Human DNA sequence *** SEQUENCING IN |
| | | | PROGRESS *** from clone (79790 nt) 9.5e-24 |
| ugs183 | AI521602 | AL034550 | to65e01.x1 NCI_CGAP_Gas4 Homo sapiens |
| | | | cDNA clone IMAGE:2183160 3′ (434 nt) 9.5e-09 |
| ugs200 | AI525836 | C83432 | PT1.3_06_D04.r tumor1 Homo sapiens cDNA 5′, |
| | | | mRNA sequence. March 1999 (913 nt) 6.8e-16 |
| ugs236 | AI346524 | L27758 | qp51d11.x1 NCI_CGAP_Co8 Homo sapiens |
| | | | cDNA clone IMAGE: 1926549 3′ (702 nt) 1.4e-29 |
| | | | Normalized Library ESTs (Non-Human) |
| ua1f3fr | AI407830 | X60435 | EST236120 Normalized rat ovary, Bento Soares |
| | | | Rattus sp cDNA clone (553 nt) 6.2e-21 |
| ua1a4r | AI510687 | X60435 | vx91h09.y1 Soares 2NbMT Mus musculus cDNA |
| | | | clone IMAGE:1282625 5′, (446 nt) 1e-22 |
| ug004rcon | AI103952 | X60435 | EST213241 Normalized rat heart, Bento Soares |
| | | | Rattus sp. cDNA clone (522 nt) 6.5e-41 |
| ug0l0rcon | AI071801 | X60435 | UI-R-C2-nj-h-11-O-UI.s1 UI-R-C2 Rattus |
| | | | norvegicus cDNA clone UI-R from 8-day embryo |
| | | | normalized library (218 nt) 3.2e-22 |
| ug017rcon | AI177121 | X60435 | EST220728 Normalized rat ovary, Bento Soares |
| | | | Rattus sp. cDNA clone (362 nt) 6.7e-36 |
| ug022rcon | AI415663 | X60435 | mc65f04.x1 Soares mouse embryo NbME13.5 |
| | | | 14.5 Mus musculus cDNA clone (456 nt) 2.7e-54 |
| ugo24rcon | AI337885 | X60435 | qt34d08.x1 Soares_pregnant_uterus_NbHPU |
| | | | Homo sapien cDNA clone I(516 nt) 7e-80 |
| ugo43rcon | AI465001 | AF085906 | vw55b07.y1 Soares mouse mammary gland |
| | | | NMLMG Mus musculus cDNA clone (470 nt) |
| | | | 3.6e-3 1 |
| ug091rcon | AI406675 | X60435 | EST234962 Normalized rat ovary, Bento Soares |
| | | | Rattus sp. cDNA clone (489 nt) 2.4e-15 |
| *ug096ors | AI556610 | U68380 | UI-R-C2p-ri-d-03-0-UI.s1 UI-R-C2p Rattus |
| | | | norvegicus cDNA clone (431 nt) 3e-25 |
| ug144 | AI463953 | L27758 | vw67d10.y1 Stratagene mouse heart #937316 Mus |
| | | | musculus cDNA clone (448 nt) 5.3e-59 |
| ug161 | AI179287 | L27758 | EST222980 Normalized rat spleen, Bento Soares |
| | | | Rattus sp. cDNA clone (559 at) 2.1e-37 |
| ug162 | AI454888 | L27758 | UI-R-C2p-q1-e-06-0-UI.s1 UI-R-C2p Rattus |
| | | | norvegicus cDNA clone (375 nt) 1.5e-45 |
| ug163rcon | AI462455 | L27758 | ub73a04.x1 Soares mouse mammary gland |
| | | | NMLMG Mus musculus cDNA clone (459 nt) |
| | | | 9.6e-92 |
| ug169rcon | AI555802 | L27758 | UI-R-C2p-qz-e-01-0-UI.s1 UI-R-C2p Rattus |
| | | | norvegicus cDNA clone (515 nt) 5e-100 |
| ug183rcon | AI466568 | J05677 | vx79f07.y1 Soares 2NbMT Mus musculus cDNA |
| | | | clone IMAGE:1281445 5′, (180 nt) 4.2e-41 |
| ug212 | AI179029 | J05677 | EST222711 Normalized rat spleen, Bento Soares |
| | | | Rattus sp. cDNA clone (464 nt) 3.9e-16 |
| ug221 | AI462455 | J05677 | ub73a04.x1 Soares mouse mammary gland |
| | | | NMLMG Mus musculus cDNA clone (459 nt) |
| | | | 2.8e-83 |
| ug226 | AI386220 | J05677 | mq66d04.y1 Soares 2NbMT Mus musculus |
| | | | cDNA clone IMAGE: 583687 5′s (608 nt) 6.2e- |
| | | | 91 |
| ug237 | AI467092 | J05677 | vd64a07.x1 Knowles Solter mouse blastocyst B1 |
| | | | Mus musculus cDNA clone (358 nt) 2.4e-74 |
| ug255 | AA963280 | AU043179 | UI-R-E1-gh-h-04-0-UI.s1 UI-R-E1 Rattus |
| | | | norvegicus cDNA clone UI-R (409 nt) 1.2e-43 |
| ug280 | AI551859 | 937311 | vo93h08.x1 Soares mouse mammary gland |
| | | | NbMMG Mus musculus cDNA clone (348 nt) |
| | | | 1.4e-48 |
| ug301 | AI414548 | D21852 | ma46c09.x1 Soares mouse p3NMF19.5 Mus |
| | | | musculus cDNA clone IMAGE: (3396 nt) 2.2e-67 |
| ug318 | AU051101 | AU051101 | Sugano mouse brain mncb Mus musculus cDNA |
| | | | clone MNCb-152918 nt 1.Se-31 |
| ug342 | AA782146 | AU051101 | ai48f10.s1 Soares_parathyroid_tumor_NbHPA |
| | | | Homo sapiens cDNA clone (421 nt) 2.5e-32 |
| ug345 | AI059369 | AU051101 | UI-R-C1-1d-e-09-0-UI.s1 UI-R-C1 Rattus |
| | | | norvegicus cDNA clone UI-R (379 nt) 2.4e-24 |
| ug352 | AI536347 | AU051101 | ma93g05.y1 Soares mouse p3NMF19.5 Mus |
| | | | musculus cDNA clone IMAGE: (3638 nt) 2.7e- |
| | | | 12 |
| *ug357 | AI428736 | AU051101 | vv49a04.y1 Soares 2NbMT Mus musculus cDNA |
| | | | clone IMAGE:1225710 5′(463 nt) 1.8e-90 |
| ug358 | AI071119 | AU051101 | UI-R-C2-mt-a-08-0-UI.s1 UI-R-C2 Rattus |
| | | | norvegicus cDNA clone UI-R (369 nt) 7.9e-36 |
| ug359 | AA998224 | AU051101 | UI-R-C0-ib-d-09-0-UI.s1 UI-R-C0 Rattus |
| | | | norvegicus cDNA clone UI-R (328 nt) 5.6e-36 |
| ug383 | AI227819 | AU051101 | EST224514 Normalized rat brain, Bento Soares |
| | | | Rattus sp. cDNA clone (395 nt) 3.4e-49 |
| ug384 | AA963776 | AU051101 | UI-R-E1-gk-f-01-0-UI.s1 UI-R-E1 Rattus |
| | | | norvegicus cDNA clone UI-R (443 nt) 1.4e-24 |
| ug388 | AI011736 | AU051101 | EST206187 Normalized rat ovary, Bento Soares |
| | | | Rattus sp. cDNA clone (638 nt) 2.6e-43 |
| ug393 | AI547803 | AU051101 | UI-R-C3-sk-a-09-0-UI.s1 UI-R-C3 Rattus |
| | | | norvegicus cDNA clone UI-R (513 nt) 1.2e-29 |
| ug396 | AI347278 | AU051101 | tc05d06.x1 NCI_CGAP_Co16 Homo sapiens |
| | | | cDNA clone IMAGE: 3′, mRNA (465 nt) 1.3e-24 |
| ug399 | AI315959 | AJ010597 | uj28b10.y1 Sugano mouse kidney mkia Mus |
| | | | musculus cDNAD clone IMAGE (642 nt) 5e-46 |
| ug404 | AI314760 | AJ010597 | uj28d10.x1 Sugano mouse kidney mkia Mus |
| | | | musculus cDNAD clone IMAGE (686 nt) 1.2e-35 |
| ug421 | AI102962 | U05013 | EST212251 Normalized rat embryo, Bento Soares |
| | | | Rattus sp. cDNA clone (509 nt) 1.6e-67 |
| ug434 | AI323275 | U05013 | mp96d04.y1 Soares 2NbMT Mus musculus |
| | | | cDNA clone IMAGE:577063 5′ (487 nt) 3e-09 |
| ug436 | AI503731 | U05013 | vk81e11.x1 Knowles Solter mouse 2 cell Mus |
| | | | musculus cDNA clone IMAGE (496 nt) 1.4e-17 |
| *ug440rs | AI465094 | U05013 | vw65h07.y1 Stratagene mouse heart#937316Mus |
| | | | musculus cDNA clone (425 nt) 1.4e-47 |
| ug456 | AI451948 | U05013 | mp75e08.x1 Soares 2NbMT Mus musculus cDNA |
| | | | clone IMAGE:575078 3′,374 nt 5.7e-72 |
| ug471 | AI071177 | Z22532 | UI-R-C2-my-d-06-0-UI.s1 UI-R-C2 Rattus |
| | | | norvegicus cDNA clone UI-R (472 nt) 2.2e-51 |
| *ug482 | AI230701 | Z22532 | EST227396 Normalized rat embryo, Bento Soares |
| | | | Rattus sp. cDNA clone (420 nt) 2.3e-23 |
| ug496 | AI172198 | Z22532 | EST218195 Normalized rat muscle, Bento Soares |
| | | | Rattus sp. cDNA clone (642 nt) 1.4e-20 |
| ug504 | AI503116 | Z22532 | vm94a04.x1 Knowles Solter mouse blastocyst B1 |
| | | | Mus musculus cDNA clone (385 ut) 6e-69 |
| *ug505ors | AI314057 | Z22532 | uj25a12.x1 Sugano mouse kidney mkia Mus |
| | AA673668 | | musculus cDNA clone IMAGE (635 nt) 3.5e-45 |
| | | | or 10667012 vo57h10.r1 Soares mouse |
| | | | mammary gland NbMMG Mus musculus cDNA |
| | | | clo 547 at 6.6e-24 |
| ug519 | AI463740 | Z22532 | va16g07.y1 Soares mouse lymph node NbMLN |
| | | | Mus musculus cDNA clone (526 at) 1.5e-13 |
| ugs006 | AI045419 | Z22532 | UI-R-C1-kg-f-08-0-UI.s1 UI-R-C1 Rattus |
| | | | norvegicus cDNA clone UI-R (319 nt) 1.1e-07 |
| ugs039 | AI236146 | Z22532 | EST232708 Normalized rat ovary, Bento Soares |
| | | | Rattus sp. cDNA clone (612 nt) 1.6e-44 |
| ugs046 | AI235046 | Z22532 | EST231608 Normalized rat ovary, Bento Soares |
| | | | Rattus sp. cDNA clone (484 at) 3.5e-30 |
| ugs048 | AI412981 | Z22532 | EST241281 Normalized rat brain, Bento Soares |
| | | | Rattus sp. cDNA clone (510 nt) 5.1e-23 |
| ugs077 | AA924175 | AC004531 | UI-R-E0-bp-c-02-0-UI.s3 UI-R-E0 Rattus |
| | | | norvegicus cDNA clone UI-R (463 at) 2e-12 |
| ugs085 | AI551460 | M74555 | mp87c07.y1 Soares 2NbMT Mus musculus cDNA |
| | | | clone IMAGE:576204 5′, (406 at) 3.4e-33 |
| ugs092 | AI467480 | M74555 | vd57h06.x1 Knowles Solter mouse blastocyst B1 |
| | | | Mus musculus cDNA clone (398 nt) 2e-12 |
| ugs093 | AI482527 | M74555 | vg49d04.x1 Soares mouse mammary gland |
| | | | NbMMG Mus musculus cDNA clone (420 nt) |
| | | | 7.2e-52 |
| ugs103 | AI391039 | M74555 | mc10h07.y1 Soares mouse p3NMF19.5 Mus |
| | | | musculus cDNA clone IMAGE: (3553 at) 1.2e-12 |
| ugs120 | AA933159 | L07096 | UI-R-E0-cz-e-07-0-UI.s1 UI-R-E0 Rattus |
| | | | norvegicusD cDNA clone UI-R283 nt 5.5e-30 |
| ugs122 | AU051628 | AU051628 | Sugano mouse brain mncb Mus musculus cDNA |
| | | | clone MNCb-2261 (781 at) 5.2e-12 |
| ugs129 | AI555441 | AU051628 | UI-R-C2p-qp-h-10-0-UI.s1 UI-R-C2p Rattus |
| | | | norvegicus cDNA clone UI (377 at) 7.5e-22 |
| ugs136 | AI227835 | AU051628 | EST224530 Normalized rat brain, Bento Soares |
| | | | Rattus sp. cDNA clone (552 at) 2.8e-21 |
| ugs140 | AI466881 | U58105 | mz55d06.y1 Barstead mouse pooled organs |
| | | | MPLRB4 Mus musculus cDNA (484 at) 1.6e-14 |
| ugs143 | AI481908 | U58105 | vh18g12.x1 Soares mouse mammary gland |
| | | | NbMMG Mus musculus cDNA clone (267 nt) |
| | | | 2.2e-25 |
| *ugs148 | AI415455 | U58105 | mc57e02.x1 Soares mouse embryo NbME13.5 |
| | | | 14.5 Mus musculus cDNA clone (424) nt 9.5 e-45 |
| ugs175 | AI510452 | U58105 | mp96g07.y1 Soares 2NbMT Mus musculus cDNA |
| | | | clone IMAGE:577116 5′(435 nt) 7.4e-46 |
| ugs199 | AA818528 | C83432 | UI-R-A0-au-d-06-0-UI.s1 UI-R-A0 Rattus |
| | | | norvegicus cDNA clone UI-R (611 nt) 2.7e-30 |
| ugs202 | AI316156 | L27758 | uj25fD8.y1 Sugano mouse kidney mkia Mus |
| | | | musculus cDNA clone IMAGE (663 nt) 9.1e-49 |
| ugs214 | AI505916 | L27758 | vk69c10.x1 Knowles Salter mouse 2 cell Mus |
| | | | musculus cDNA clone IMAGE (587 nt) 3.7e-12 |
| ugs221 | AI463931 | L27758 | vw70b08.y1 Stratagene mouse heart#937316Mus |
| | | | musculus cDNA do (402 nt) 3.3e-18 |
| | | | Unknown |
| *ug120 | AC005835 | U68380 | Mus musculus clone UWGC:mbac82 from 14D1- |
| | | | D2, T-Cell Receptor Alpha (79548 nt) 3.2e-20 OR |
| | | | WORKING DRAFT SEQUENCE, 17 unordered |
| | | | pieces. March 1999 (79545 nt) 9e-18 |
| ug276 | AI504762 | 937311 | v117h02.x1 Stratagene mouse T cell Mus |
| | | | musculus cDNA clone (504 nt) 8e-111 |
| ug298 | AC005992 | D21852 | *** SEQUENCING IN PROGRESS *** HTGS |
| | | | phase 1, 13 unordered pieces.(79759 nt) 2.8e-10 |
| ug428 | AC004821 | U05013 | *** SEQUENCING IN PROGRESS *** Homo |
| | | | sapiens clone DJ0098O22; HTGS (79478 nt) |
| | | | 2.7e-22 |
| ugs198 | C83432 | C83432 | rabbit corneal endothelial cell Oryctolagus |
| | | | cuniculus cDNA clone (361 nt) 1.3e-39 |
| ug138 | BIACOMGEN | L27758 | Birmingham IncP-alpha plasmidR18, R68, RK2, D |
| | | | RP1, RP4co (60099 nt)2.2e-10 |
| ug239 | BIACOMGEN | L27758 | Birmingham IncP-alpha plasmidR18, R68, RK2, D |
| | | | RP1, RP4co (60099 nt) 4.9e-32 |
| ug315 | BIACOMGEN | L27758 | Birmingham IncP-alpha plasmidR18, R68, RK2, |
| | | | RP1, RP4co (60099 nt)1.4e-18 |
| ugs201 | BIACOMGEN | L27758 | Birmingham IncP-alpha plasmidR18, R68, RK2, |
| | | | RP1, RP4co (60099 nt)4e-29 |
| ua1e3r | AB002387 | U49739 | Human mRNA for KIAA0389 gene, complete |
| | | | cds. June 1997 (5212 nt) 6.1e-29 |
| ua1g4r | HUMORF05 | D14661 | Human mRNA for KIAA0105 gene, complete |
| | | | cds. July 1997 (1622 nt) 6.4e-49 |
| ug282 | HUMORFIA | D21852 | Human mRNA for KIAA0029 gene, partial cds. |
| | | | July 1997 (4272 nt) 3.4e-08 |

GenBank: All listed databases except EST.

Table 5 presents the results of the library analysis of 787 cDNA UGS-derived ESTs using the GenBank expressed sequence tag database.

TABLE 5
Results of the library analysis of 787 cDNA UGS-derived ESTs
using the GenBank expressed sequence tag database

| Clone | Locus | Acc. # | Identity (Source Tissue or Near Match) |
|---|---|---|---|
| | | | Protein Kinases/Phosphatases |
| ug003 | | AA552488 | nk12e05.s1 NCI_CGAP_Co2 Homo sapiens cDNA clone |
| | | | IMAGE:1013312 3′similar to gb:L07395 Protein |
| | | | Phosphatase PP1-Gamma Catalytic Subunit |
| | | | (HUMAN);contains element MER35 repetitive element |
| | | | mRNA sequence (600 nt) 7.5e-07 |
| | | | Growth Factors Induced |
| ugs068 | | H33545 | 10667012 EST109665 Rat PC-12 cells, NGF-treated 9 |
| | | | days Rattus sp. cDNA clone (291 nt)3.1e-33 |
| | | | Transcription Factors/Co-factors |
| ug171rcon | | AI060775 | d08016 ub43e04.r1 Soares 2NbMT Mus musculus cDNA |
| | | | clone IMAGE:1380510 5′end similar to WP:F13H6.1 |
| | | | CE09373 Zinc-Finger Protein; (388 nt) 8.1e-19 |
| | | | Egg (Fertilized) |
| ug186rcon | | | C88119 Mouse fertilized one-cell-embryo cDNA Mus |
| | | | musculus cDNA clan 567 nt 2.4e-46 |
| | | | Egg (Unferrtilized) |
| ug185rcon | | AU023398 | AU023398 Mouse unfertilized egg cDNA Mus |
| | | | musculus cDNA clone J043 585 nt 9.5e-15 |
| | | | Two-Cell Embryo |
| ua1c1 | | AA536741 | vj88c04.r1 Knowles Solter mouse 2 cell Mus musculus |
| | | | cDNA clone IM 603 nt 1.2e-38 |
| ug110rcon | | AU016170 | Mouse two-cell stage embryo cDNA Mus musculus cDNA |
| | | | clone 596 nt 1.6e-07 |
| | | | Blastocyst |
| ugs233 | | AA590398 | 937311 vm16b09.r1 Knowles Solter mouse blastocyst B1 |
| | | | Mus musculus cDNA c 521 at 2.1e-29 |
| | | | Fetus (7.5d pc) |
| ug005rcon | | AA120195 | mn34h12.r1 Beddington mouse embryonic region Mus |
| | | | musculus cDNA clone 7.5d pc (485 nt) 2.9e-42 |
| ug216 | | AA409017 | 10667012 EST03497 Mouse 7.5 dpc embryo |
| | | | ectoplacental cone cDNA library Mus 475 nt 3.2e-26 |
| | | | Fetus (13.5-14.5d pc) |
| ua1f4f | | AA051759 | mj54d09.r1 Soares mouse embryo NbME 13.5 14.5 Mus |
| | | | musculus cDNA c1 455 nt 9.5e-27 |
| | | | Fetus (15.5d pc) |
| ug210 | | AA288823 | 10667012 mr51a03.r1 Life Tech mouse embryo 15 |
| | | | 5dpc Mus musculus c 212 nt4.8e-19 |
| | | | Fetus-Human (9 wk) |
| ug398 | | AA451660 | 10667012 zx43f06.r1 |
| | | | Soares_total_fetus_Nb2HF8_9wk Homo sapiens cDNA |
| | | | clone I (471 nt) 2.3e-54 |
| | | | Brain |
| ug223 | | AA982689 | 10667012 uh12b11.r1 Soares mouse hypothalamus |
| | | | NMHy Mus musculus cDNA clone 243 nt 1.4e-58 |
| ug233 | | Z46187 | 10667012 HSC26A061 normalized infant brain |
| | | | cDNA Homo sapiens cDNA clone c-26 294 nt 6.4e-12 |
| ugs196 | | R12838 | 937311 yf57g11.r1 Soares infant brain 1NIB Homo |
| | | | sapiens cDNA clone IMAGE:2 410 nt 9.6e-34 |
| | | | Breast |
| ug132 | R75784 | AU016170 | y121d10.r1 Soares breast 2NbHBst Homo sapiens cDNA |
| | | | clone IMAGE:1588 616 nt 3.9e-10 |
| ug443 | | AA463093 | 10667012 vf92h09.r1 Soares mouse mammary gland |
| | | | NbMMG Mus musculus cDNA clo 445 nt 1.5e-15 |
| | | | Heart |
| ug009rcon | | AA512195 | vj21d02.r1 Soares mouse NbMH Mus musculus cDNA |
| | | | clone from heart IMAGE:922371 (434 nt) 1.1e-31 |
| | | | Liver/Spleen |
| ug143 | R94675 | | d08016 yq42g09.r1 Soares fetal liver/spleen 1NFLS |
| | | | Homo sapiens cDNA clone (396 nt) 1.4e-14 |
| ug191rcon | | AA177621 | C88119 mt32e09.r1 Soares mouse 3NbMS Mus musculus |
| | | | cDNA clone from 4wk spleen IMAGE:62279 (517 nt) |
| | | | 2.2e-66 |
| | | | Macrophage/T cells |
| ug025rcon | | AA896565 | vx63h11.r1 Stratagene mouse macrophage #937306 Mus |
| | | | musculus cDNA (542 nt) 2.4e-42 |
| ug431 | | AA896311 | 10667012 vy13b07.r1 Stratagene mouse macrophage |
| | | | #937306 Mus musculus cDN (371 nt)9.5e-41 |
| ug463 | | AA981302 | 10667012 vx60a04.r1 Stratagene mouse macrophage |
| | | | #937306 Mus musculus cDN (423 nt)2.6e-61 |
| ugs108 | | AA655870 | 937311 vs41h04.r1 Stratagene mouse T cell Mus |
| | | | musculus cDNA clone (403 nt) 1.3e-55 |
| | | | Myotubes |
| ug314 | | AA815998 | 10667012 vr14b11.r1 Barstead mouse myotubes |
| | | | MPLRBS Mus musculus cDNA clone (603 nt) 1.8e-36 |
| ug343 | | AA754682 | 10667012 vu20e09.r1 Barstead mouse myotubes |
| | | | MPLRB5 Mus musculus cDNA clone (472 nt) 3 .9e-59 |
| ugs161 | | AA521515 | 937311 vi07b05.r1 Barstead mouse myotubes MPLRB5 |
| | | | Mus musculus cDNA clone (602 nt) 7.7e-59 |
| | | | Ovary/Female Reproductive |
| ug516 | | AA429173 | 10667012 zv49e04.r1 Soares ovary tumor NbHOT Homo |
| | | | sapiens cDNA clone IMAGE (492 bt) 2e-14 |
| ugs070 | | AA338077 | 10667012 EST42893 Endometrial tumor Homo sapiens |
| | | | cDNA 5′end similar to small nuclear ribonucleoprotein, |
| | | | polypeptide C, mRNA sequence 293 nt 4.9e-29 |
| | | | Retina |
| *ug491ft | | W26371 | 10667012 26f7 Human retina cDNA randomly primed |
| | | | sublibrary Homo sapiens cDNA (621 nt) 8.5e-29 |
| | | | Testis/Male Reproductive |
| ug064rcon | | AA139248 | mr69b12.r1 Stratagene mouse testis #937308 Mus |
| | | | musculus cDNA c1 (347 nt)1e-10 |
| ug137rcon | T19209 | | d08016 d08016t Testis 1 Homo sapiens cDNA clone 5′ |
| | | | end, mRNA sequence (397 nt) 2.9e-12 |
| ug184rcon | | AI115344 | d08016 uh84a11.r1 Soares mouse urogenital ridge |
| | | | NMUR Mus musculus cDNA c (417 nt) 7.7e-69 |
| | | | Thymus |
| ugo30rcon | | AA120338 | mp89a02.r1 Soares 2NbMT Mus musculus cDNA clone |
| | | | from 4 wk thymus IMAGE:576362 5′, (334 nt) 2.5e-39 |
| ug295 | | AA209880 | 10667012 mu40c07.r1 Soares 2NbMT Mus musculus |
| | | | cDNA clone 4wk thymus IMAGE:641868 5′, (501 nt) |
| | | | 3.8e-106 |
| ug418 | | AI049035 | 10667012 ub39b04.r1 Soares 2NbMT 4 wks Mus |
| | | | musculus cDNA clone IMAGE: 1380079 5′cDNA from |
| | | | Thymus, (530 nt) 2.1e-71 |
| ugs011 | | AI060722 | 10667012 ub42h03.r1 Soares 2NbMT Mus musculus |
| | | | cDNA clone from 4wk fetal thymus IMAGE:1380437 |
| | | | 5′end, (457 nt) 2.9e-38 |

GenBank: Expressed Sequence Tags Database May 3, 1999

Table 6 presents a summary of the urogenital sinus clone unknowns.

TABLE 6
List of Urogenital Sinus clone unknowns

| | |
|---|---|
| UGS Clone Unknowns | N = 157 |

| | | | |
|---|---|---|---|
| ua1a6f | ua1b4 | ua1d2 | ua1e5r |
| ua1f1r | ua1f6 | ua1g5f | ua1h4 |
| ug003meld | ug006rcon | ug008rcon | ug012rcon |
| ug015rcon | ug018rcon | ug020r2 | ug026rcon |
| ug032rcon | ug041rcon | ug044rcon | ug046 |
| ug047rcon | ug054 | ug071rcon | ug074rcon |
| ug075rcon | ug079rcon | ug085rcon | ug090rcon |
| ug096rcon | ug097rcon | ug098rcon | *ug106rcon |
| ug107rcon | ug112 | ug113rcon | ug115rcon |
| ug117 | ug118 | ug121 | ug123 |
| ug128 | ug130r2 | ug146 | ug148 |
| ug151rcon | ug152rcon | ug154rcon | ug173rcon |
| ug175rcon | ug177rcon | ug189rcon | ug190rcon |
| ug203 | ug208 | ug217 | ug227 |
| ug229 | ug230 | ug241 | ug242 |
| ug247 | ug250 | ug253 | ug254f |
| ug259 | ug262 | ug269 | ug272 |
| ug273 | ug274 | ug277f | ug285 |
| ug288 | ug294 | ug299 | ug305 |
| *ug320 | ug322 | ug330 | ug331 |
| ug332 | ug338 | ug339 | ug341 |
| ug344 | ug351 | *ug353 | ug360 |
| ug368 | ug372 | ug387 | ug403 |
| ug417 | ug424 | ug430 | ug432 |
| ug433 | ug437 | ug439 | ug446 |
| ug452 | ug466 | ug473 | *ug484 |
| ug492 | ug495 | ug500 | ug501 |
| ug508 | ug511 | ug514 | ug520 |
| ugs001 | ugs003 | ugs009 | ugs012 |
| ugs013 | ugs014 | ugs028 | ugs034 |
| ugs035 | ugs040 | ugs041 | ugs047 |
| ugs050 | ugs051 | ugs052 | ugs054 |
| ugs066 | ugs067 | ugs071 | ugs074 |
| ugs084 | ugs086 | ugs087 | ugs088 |
| ugs111 | ugs118 | ugs121 | ugs131 |
| ugs144 | ugs150 | ugs151 | ugs156 |
| ugs163 | ugs168 | ugs172 | ugs173 |
| ugs179 | ugs182 | ugs184 | ugs187 |
| ugs193 | ugs204 | ugs212 | ugs229 |
| ugs231 | | | |

Table 7 presents the summary of the 33 clones obtained from the library contig subtraction analysis of all 787 cDNA UGS-derived ESTs cDNA clones.

TABLE 7
List of Potential Differentially Expressed UGS Clones by Database

| | | | |
|---|---|---|---|
| | | | SwissProt Match |
| | | | GTPases |
| *ug307cons | GBLP_HUMAN | P25388 | homo sapiens (human), mus musculus (mouse) |
| | | | guanine nucleotide binding protein (317 aa) |
| | | | 3.2e-57 |
| *ug308t | GBLP_HUMAN | P25388 | homo sapiens (human), mus musculus (mouse) |
| | | | guanine nucleotide binding protein (317 aa) |
| | | | 8.8e-55 |
| | | | Protein Kinases |
| *ugs186oft | CLK1_MOUSE | P22518 | mus musculus (mouse). protein kinase clk (ec |
| | | | 2.7.1.-) ( (483 aa) 5.7e-08 (see also clk-4 |
| | | | AF033566 (1549 nt) 9.7e-39) |
| | | | Ribosomal Proteins and Translation |
| *ug334 | SR14_MOUSE | P16254 | mus musculus (mouse). signal recognition |
| | | | particle 14kd (110 aa) 6.9e-42 |
| *ug354cons | RLA2_HUMAN | P05387 | homo sapiens (human). 60s acidic ribosomal |
| | | | protein p2. (115 aa) 1.2e-29 |
| | | | Transcription Factors |
| *ug277t | HXAD_AMBME | P50210 | ambystoma mexicanum (axolotl). homeotic |
| | | | protein hox-a13 (107 aa) 1.2e-34 (other locus |
| | | | 1399859 Acc #U59322) |
| | | | RNA Splicing, Binding, RNPs, etc . . . |
| *ug311cons | PSF_HUMAN | P23246 | homo sapiens (human). ptb-associated splicing |
| | | | factor (ps (707 an) 1.7e-25 |
| *ug4850rs | RNPL_HUMAN | P98179 | homo sapiens (human). putative rna-binding |
| | | | protein rnpl (157 aa) 3.1e-12 |
| | | | Peptidases, Proteinases, Isomerases, |
| | | | Transferases |
| *ug101rcon | DPP4_MOUSE | P28843 | mus musculus (mouse). dipeptidyl peptidase iv |
| | | | (ec 3.4.1) (760 aa) 5.7e-07 |
| *ug335 | NEP_RAT | P07861 | rattus norvegicus (rat). neprilysin (ec 3.4.24.11) |
| | | | (neutral endopeptidase) (749 aa) 5e-20 |
| | | | Hypothetical |
| *ug093rcon | YO11_MOUSE | P11260 | mus musculus (mouse). hypothetical protein |
| | | | orf-1137. (L1Md domain protein, repetitive |
| | | | element retroposon-like) 7/(379 aa) 4.4e-46 |
| | | | GenPent Matches |
| | | | Unknown |
| *ug371f | 1480863 | U63332 | super cysteine rich protein; SCRP Homo |
| | | | sapiens (46 aa) 1.9e-08 (expression appears |
| | | | ubiquitois) |
| | | | GenBank Primate/Rodent |
| | | | Protein Kinases |
| *ug441ors | | AF027504 | Mus musculus putative membrane-associated |
| | | | guanylate kinase 1 (Mag(919 nt) 1.8e-2 |
| | | | Structural Proteins/ECM |
| *ug102cons | RATCTTG | M80829 | Rat troponin T cardiac isoform gene, complete |
| | | | cds. September 1996 (19185 nt) 2.3e-12 (Highly repetitive |
| | | | jdr) |
| | | | Oncogenes/Tumor Suppressors/Apoptosis |
| *ug503s | | AF017989 | Mus musculus secreted apoptosis related |
| | | | protein 1(Sarp1) mRNA, c (2031 nt) 8.3e-57 |
| | | | Membrane Proteins/Receptors |
| *ug254 | MUSBA | D82019 | Mouse gene for basigin, complete cds (exon 1- |
| | | | 7). February 1997 (11763 nt) (Basigin, a new, broadly |
| | | | distributed member of the immunoglobulin |
| | | | superfamily, has strong homology with both the |
| | | | immunoglobulin V domain and the beta-chain |
| | | | of major histocompatibility complex class II |
| | | | antigen.) January 1998 (1302 nt) 5.9e-95 |
| *ug493ors | MMEZR | X60671 | M. musculus mRNA for ezrin. August 1996 (2701 nt) (A |
| | | | gene family consisting of ezrin, radixin and |
| | | | macsin. Its specific localization at actin |
| | | | filament/plasma membrane association |
| | | | sites.)2.9e-57 |
| | | | RNA Splicing, Binding, RNPs, etc . . . |
| *ug485 | MUSCIRPB | D78135 | Mus musculus mRNA for CIRP, complete cds. |
| | | | February 1998 (1256 nt) 7e-09 |
| *ug494cons | HUMASF | M72709 | Human alternative splicing factor mRNA, |
| | | | complete cds. September 1991 (1717 nt) 1.2e-27 |
| | | | X Chromosome Associated |
| *ugs194rs | | AC005859 | Homo sapiens Xp22-83 BAC GSHB-324M7 |
| | | | (Genome Systems Human BAC Lib (79502 nt) |
| | | | 3.2e-07 |
| | | | Unknown |
| *ug317 | MMU80894 | U80894 | Mus musculus CAG trinucleotide repeat |
| | | | mRNA, Transcription factor or Cadherin. (543 |
| | | | nt) 6.Se-51 |
| *ug506or | MMY17106 | Y17106 | Mus musculus transposon ETn, SELH/L3A |
| | | | strain. October 1998 (5542 nt)2.5e-73 |
| | | | GenBank All other database except ESTs |
| | | | Transcription Factors/Co-factors |
| *ug092 | GGU68380 | U68380 | Gallus gallus single-strand DNA-binding |
| | | | protein. csdp SSDP |
| | | | (sequence-specific single-stranded DNA- |
| | | | binding protein), mRNA,(1211 nt) 5.2e-85 |
| | | | Normalized Library ESTs (Non-Human) |
| *ug096ors | AI556610 | U68380 | UI-R-C2p-ri-d-03-0-UI.S1 UI-R-C2p Rattus |
| | | | norvegicus cDNA clone (431 nt) 3e-25 |
| *ug357 | AI428736 | AU051101 | vv49a04.y1 Soares 2NbMT Mus musculus |
| | | | cDNA clone IMAGE:1225710 5′(463 nt) 1.8e- |
| | | | 90 |
| *ug440rs | AI465094 | U05013 | vw65h07.y1 Stratagene mouse |
| | | | heart#9373l6Mus musculus cDNA clone (425 |
| | | | nt) 1.4e-47 |
| *ug505ors | AI314057 | Z22532 | uj25a12.x1 Sugano mouse kidney mkia Mus |
| | AA673668 | | musculus cDNA clone IMAGE (635 nt) 3.5e-45 |
| | | | or 10667012 vo57h10.r1 Soares mouse |
| | | | mammary gland NbMMG Mus musculus cDNA |
| | | | clo 547 nt 6.6e-24 |
| *ugs148 | AI415455 | U58105 | mc57e02.x1 Soares mouse embryo NbME13.5 |
| | | | 14.5 Mus musculus cDNA clone (424) nt 9.5e- |
| | | | 45 |
| | | | Unknown |
| *ug120 | AC005835 | U68380 | Mus musculus clone UWGC:mbac82 from |
| | | | 14D1-D2, T-Cell Receptor Alpha (79548 nt) |
| | | | 3.2e-20 OR WORKING DRAFT SEQUENCE, |
| | | | 17 unordered pieces. March 1999 (79545 nt) 9e-18 |
| | | | GenBank ESTs May 3, 1999 |
| | | | Retina |
| *ug491ft | | W26371 | 10667012 26f7 Human retina cDNA randomly |
| | | | primed sublibrary Homo sapiens cDNA (621 |
| | | | nt) 8.5e-29 |
| | | | Did Not Match Anything-Truly |
| | | | Unidentified |
| *ug106rcon | | | |
| *ug320 | | | |
| *ug353 | | | |
| *ug484 | | | |

Table 8 presents a summary of the library contig subtraction analysis for the 728 cDNA UGS-derived ESTs which reveals 33 differentially expressed UGS-derived EST-containing fetal prostate genes as well two potential homeobox proteins.

TABLE 8
Potentially Differentially Expressed Clones

| | | Conti | | | | | Sum | | |
|---|---|---|---|---|---|---|---|---|---|
| | Contig | g sz | Forw or | fragme | Rev or | fragme | (F + R) | | |
| Clone | (Y/N) | (bp) | T7 (bp) | nts | Sp6 (bp) | nts | (bp) | Comments | |
| ug092 | N | | 499 | FT | 594 | ORS | 1093 | GC rich on rev end | |
| ug093 | N | | 165 | FT | 349 | ORS | 514 | | |
| ug096 | N | | 96 | F | 533 | RS | 629 | GC rich on rev end | |
| ug101 | Y | 503 | | | | ORSFT | 503 | 130 bp overlap/120 bp one run | |
| ug102 | Y | 1133 | | | | ORSFT | 1133 | 63 bp overlap | |
| ug106 | N | | 312 | FT | 294 | ORS | 606 | | |
| ug120 | N | | 87 | FFT | 444 | OS | 531 | cdx-1? or T cell receptor | |
| | | | | | 168 | R | | R and OS don't align | |
| ug254 | N? | | 29 | F | 461 | ORS | 490 | 29 has pT and 461 has pA | |
| ug277 | N | | 514 | T | 409 | ORS | 923 | | hoxal3 |
| | | | 104 | F | | | | T7 and F do not align | |
| ug291 | N | | 408 | FFFT | 409 | ORS | 817 | | |
| ug307 | Y | 524 | | | | ORSFT | 524 | 160 bp Overlap/ pT on ORS | |
| ug308 | N | | 440 | T | 403 | O | 843 | | |
| | | | 77 | F | | | | pT no overlap with T7 | |
| | | | | | | ORSSP | | | |
| ug311 | Y | 704 | | | | T | 704 | 110 bp overlap | |
| ug317 | Y | 645 | | | | ORSTT | 645 | 250 bp overlap | |
| ug320 | N | | 279 | FT | 560 | ORS | 839 | | |
| ug334 | N | | 100 | FT | 572 | ORS | 672 | pT on FT | |
| ug335 | N | | 248 | T | 156 | ORS | 404 | pT on OR | |
| ug353 | N | | 92 | FT | 633 | ORS | 725 | pT on ft | |
| ug354 | Y | 445 | | | | ORSPT | 445 | pT on ORS | |
| ug357 | N | | 225 | FT | 428 | ORS | 653 | | |
| | | | | | | | | 72 bp | |
| | | | | FF(TRI) | | | | overlap/FF(TRI) pb | |
| ug371 | Y | 612 | 140 | | | ORSF | 752 | alignment | hoxd11 |

| | | | | | | | |
|---|---|---|---|---|---|---|---|
| ug440 | N | 41 | F | 217 | RS | 258 | pT on F no orig data |
| ug441 | N | 290 | FT | 338 | ORS | 628 | no orig data |
| ug482 | N | 213 | FT | 330 | ORS | 543 | |
| ug484 | N | 234 | FT | 419 | ORS | 653 | |
| ug485 | N | 153 | T | 622 | ORS | 775 | |
| ug491 | N | 603 | FT | 394 | ORS | 997 | |
| ug493 | N | 205 | FT | 263 | ORS | 468 | pT on FT |
| ug503 | N | 159 | FFTT | 310 | R | 469 | pT on FT/ |
| | | | | 276 | S | | RS do not align |
| ug505 | N | 180 | FFTT | 260 | ORSS | 440 | |
| | | | FFF(tri) | | | | |
| ug506 | N | 204 | TT | 307 | OR | 511 | pT on F |
| | | | | | | 0 | |
| ugs148 | N | 279 | OFT | 292 | RS | 571 | |
| ugs186 | N | 410 | OFT | 238 | S | 648 | |
| ugs194 | N | 391 | OFT | 490 | RS | 881 | |

These aforementioned 33 cDNA clones can be found in the accompanying tables and figures and are represented herein by the following designations: ug092, ug093, ug096, ug101, ug102, ug106,ug120, ug254, ug291,ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194.

These aforementioned 33 clones have been used herein to identify human paralogs for prostate cancer progression using the LNCaP (androgen dependent, non-tumorigenic) and lineage derived C4-2 (androgen-independent, tumorigenic metastatic to bone) cell line model. Similarly, these 728 fetal UGS-derived cDNA clones could be used to identify other human paralogs involved in the development of prostate diseases including, without limitation, prostatitis, and benign and malignant growth of the prostate gland. “Human paralogs”, as used herein, is intended to mean the human equivalent or homologous sequence.

These aforementioned 33 clones may be used to identify the aggressiveness of prostate cancer by nucleic acid hybridization techniques or via immunological detection by antisera specific to the gene product. The 33 clones may also be used to develop therapeutic modalities including: tissue- or cancer-specific gene promoters for use in gene therapy by naked DNA delivery; viral toxic gene therapy growth suppression of prostate cancer by replacement gene therapy; tissue specific gene products may also be used to develop immunotherapeutic agents using peptide specific anti-prostate cancer vaccines or adoptive immunotherapies using peptide/protein specific cytotoxic T-cells. Additional cDNA clones may be identified from the 787 UGS-derived ESTs with comparable utility.

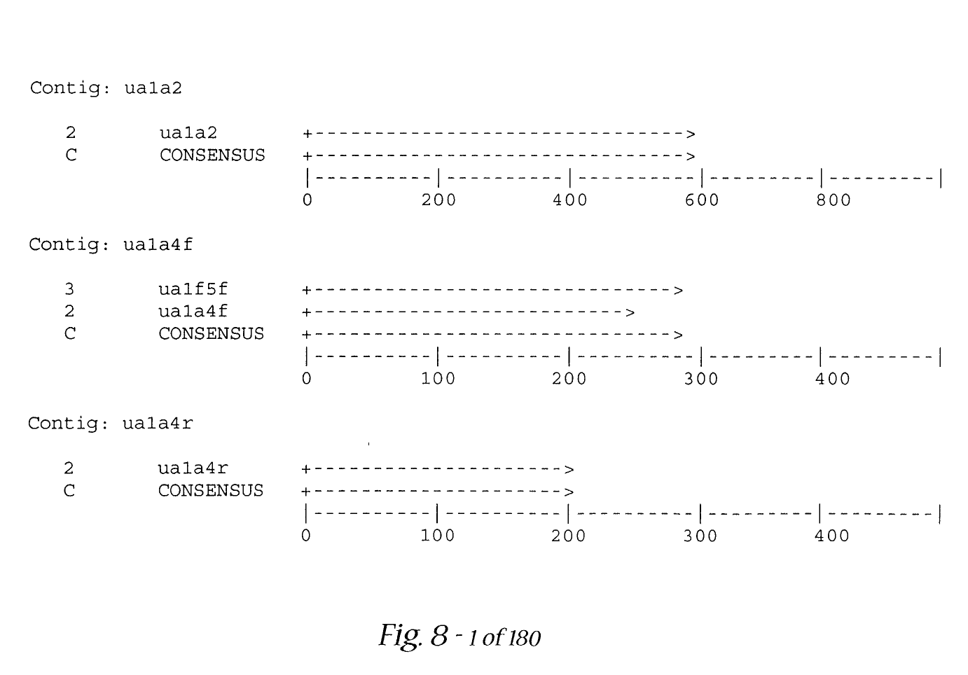

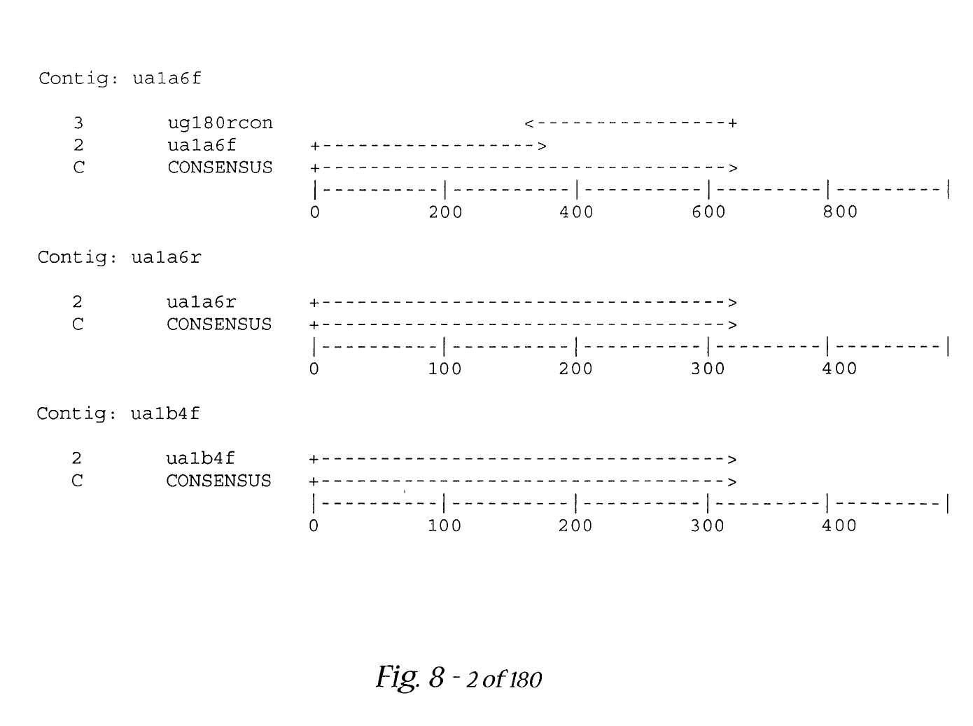

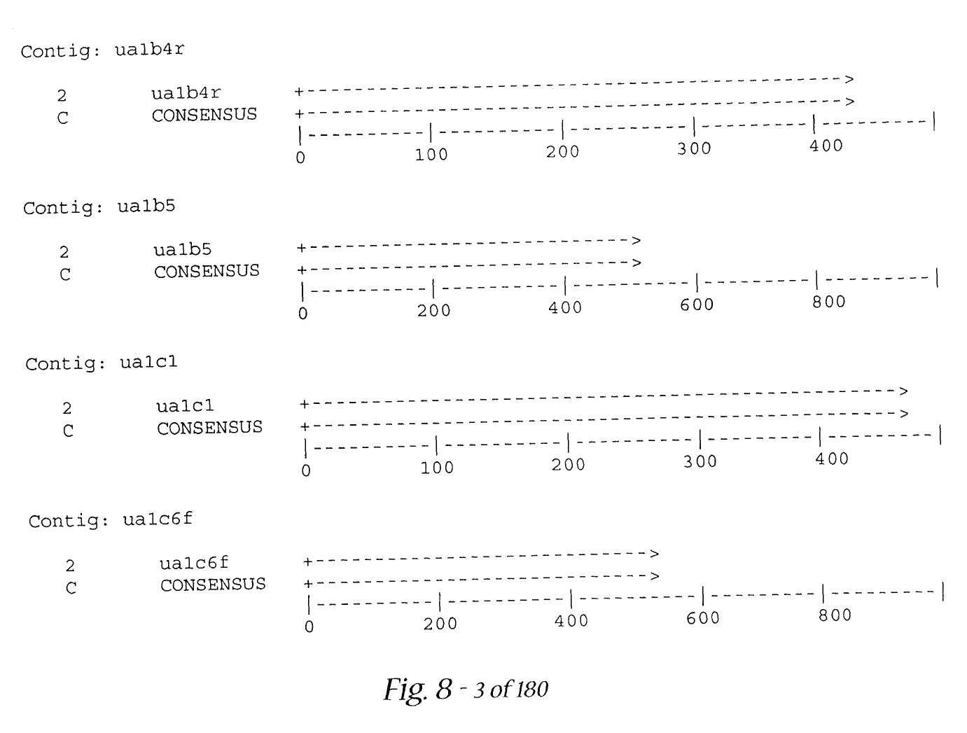

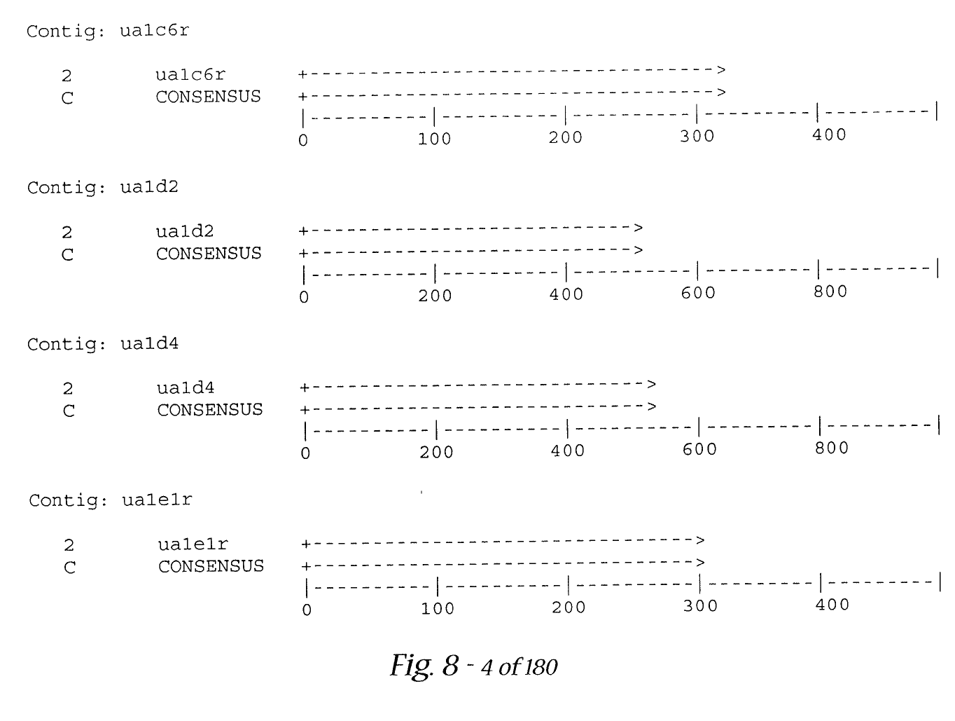

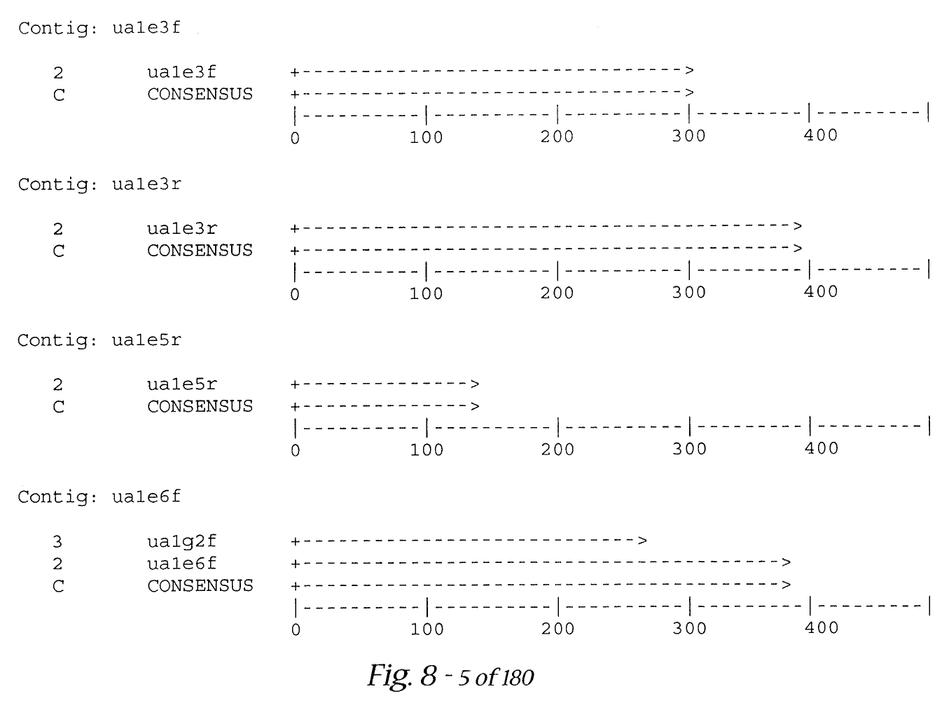

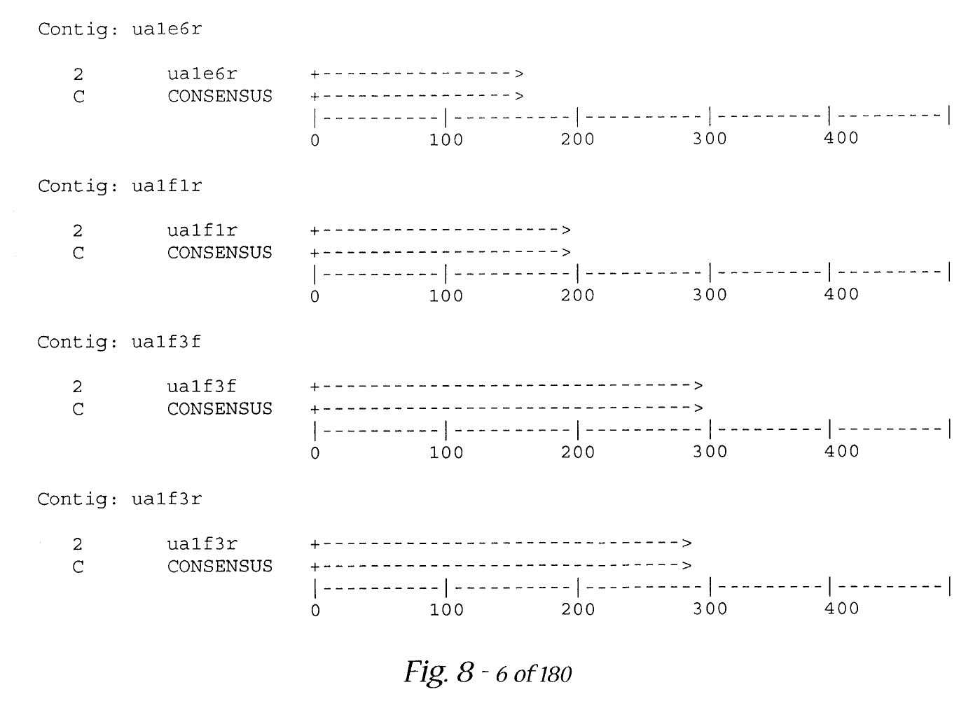

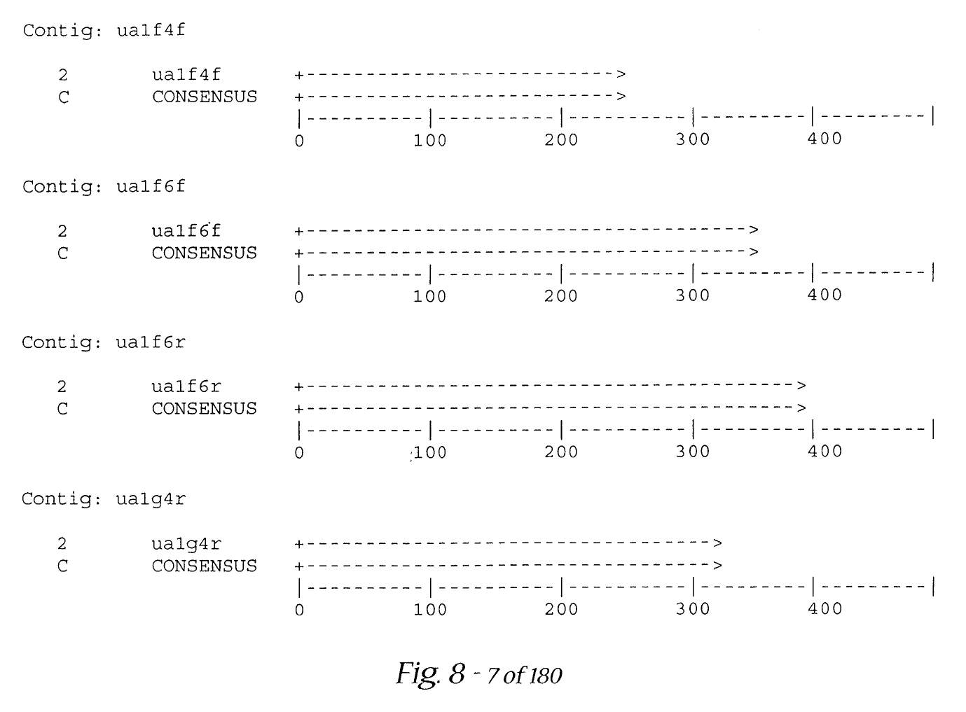

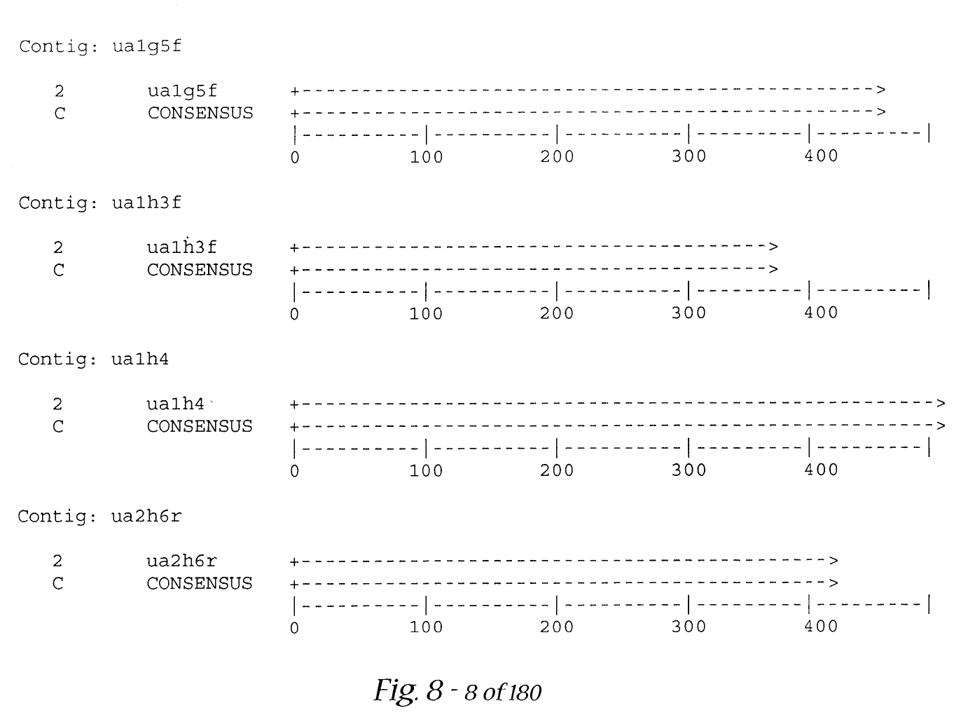

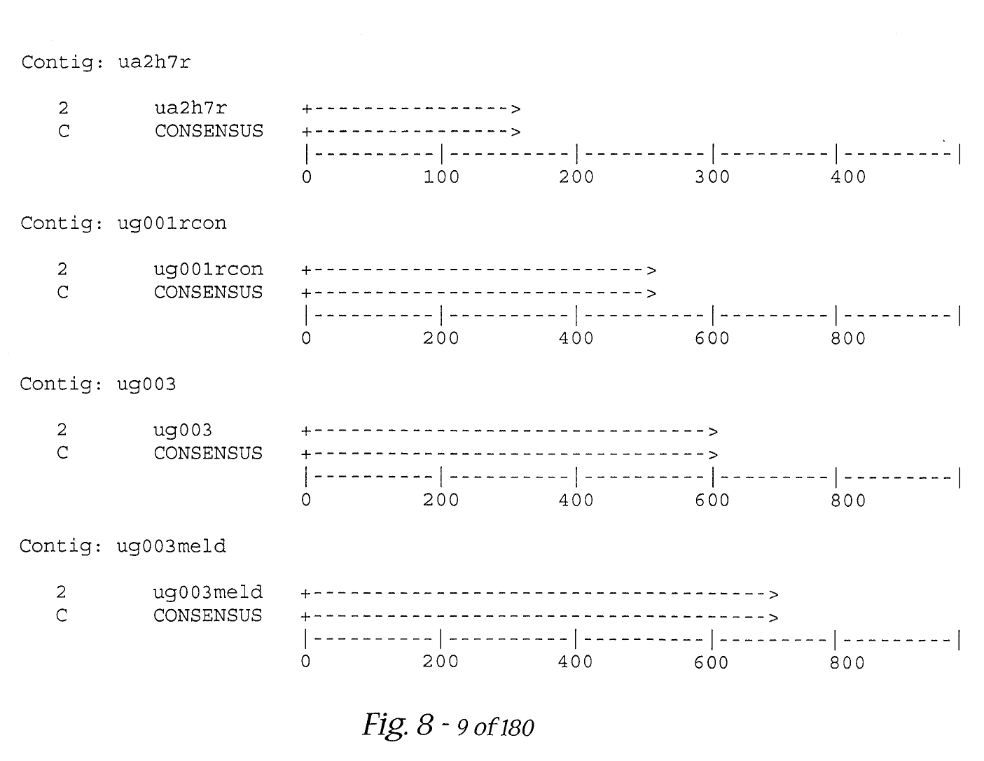

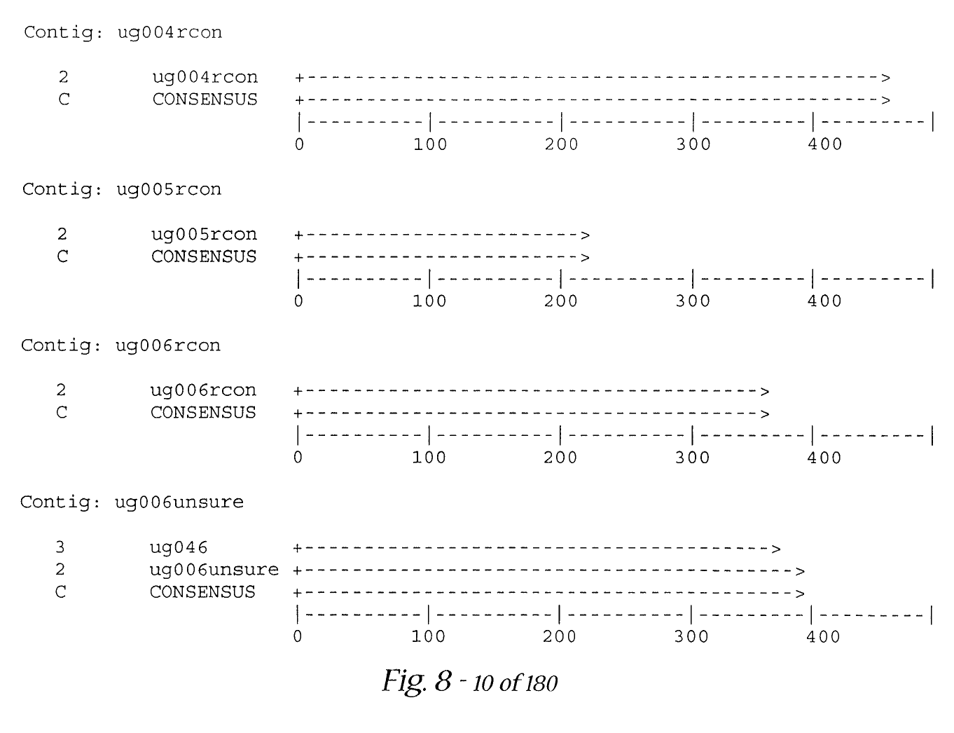

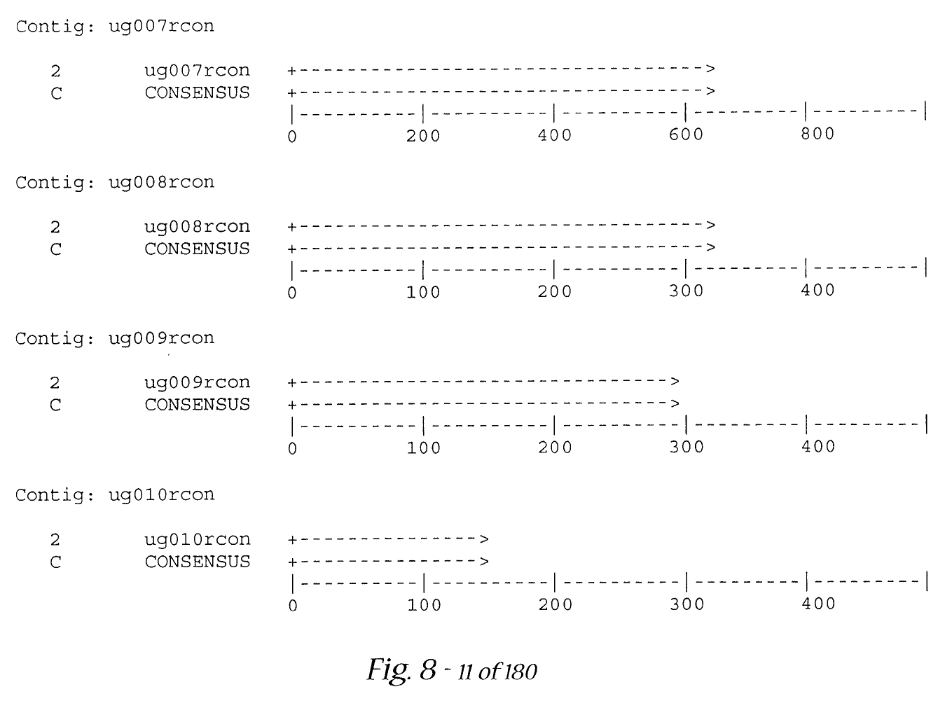

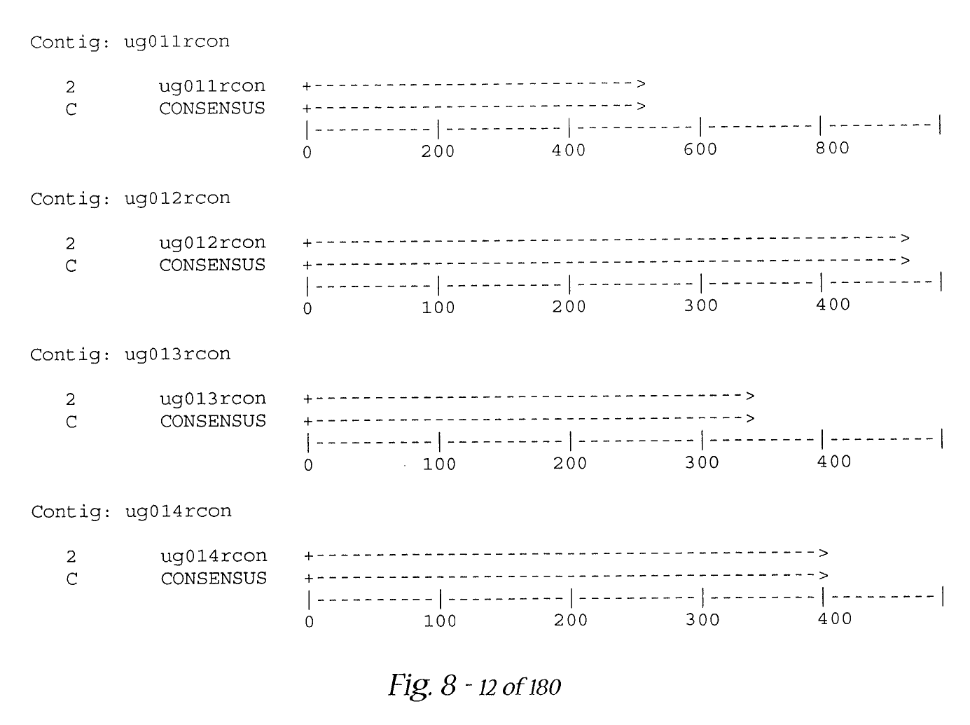

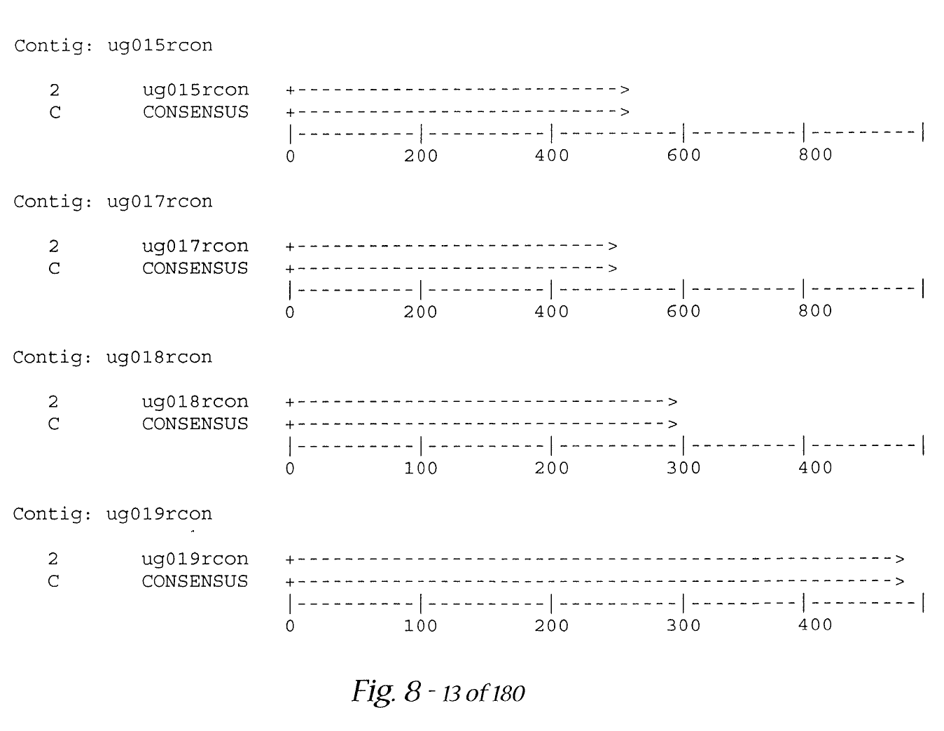

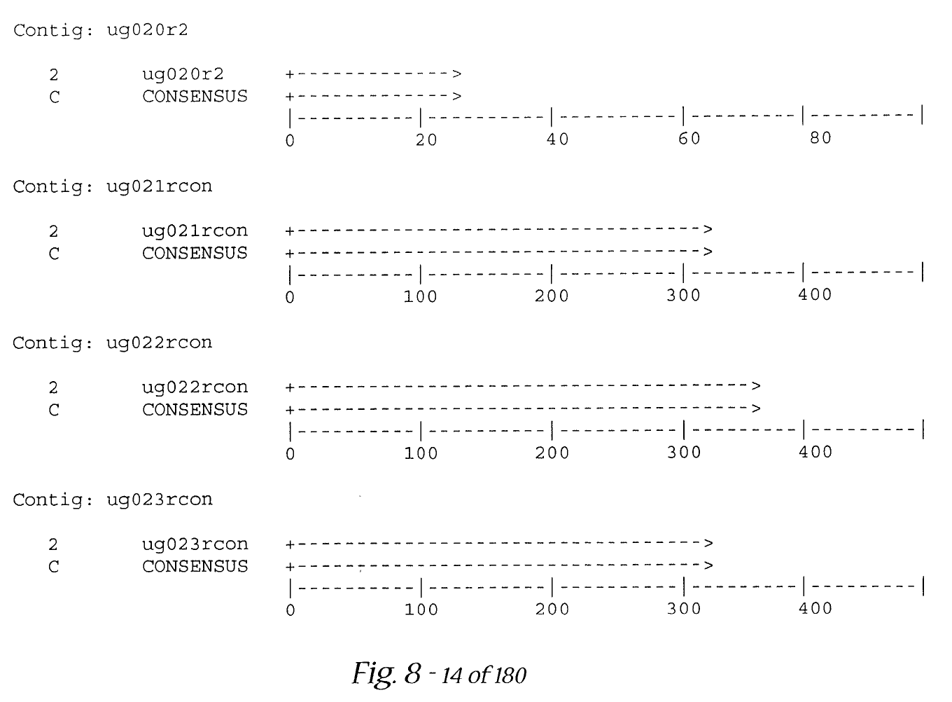

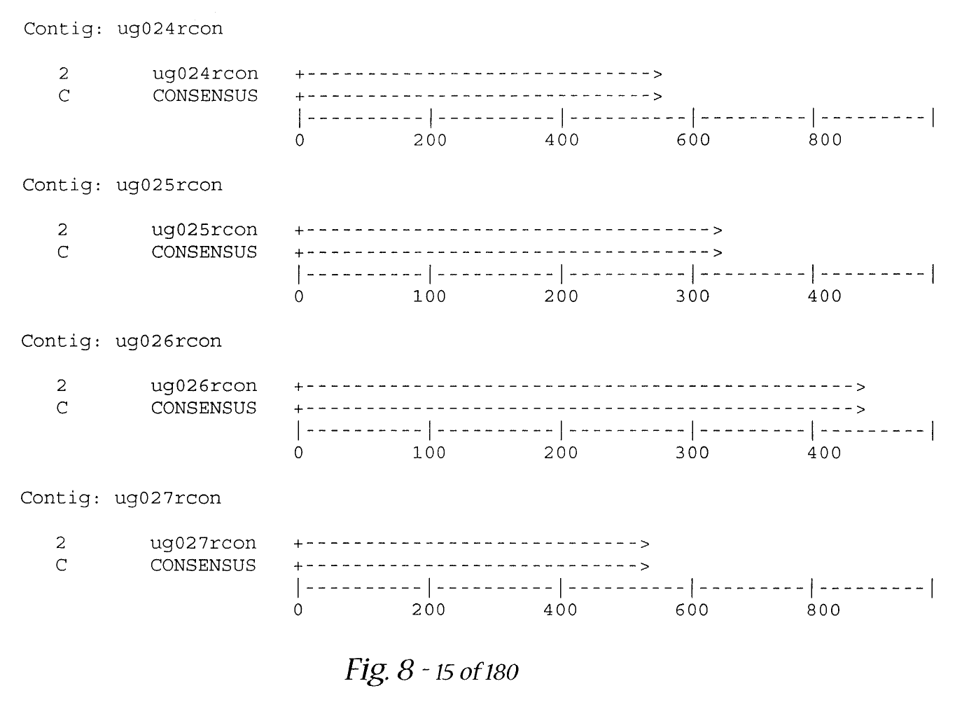

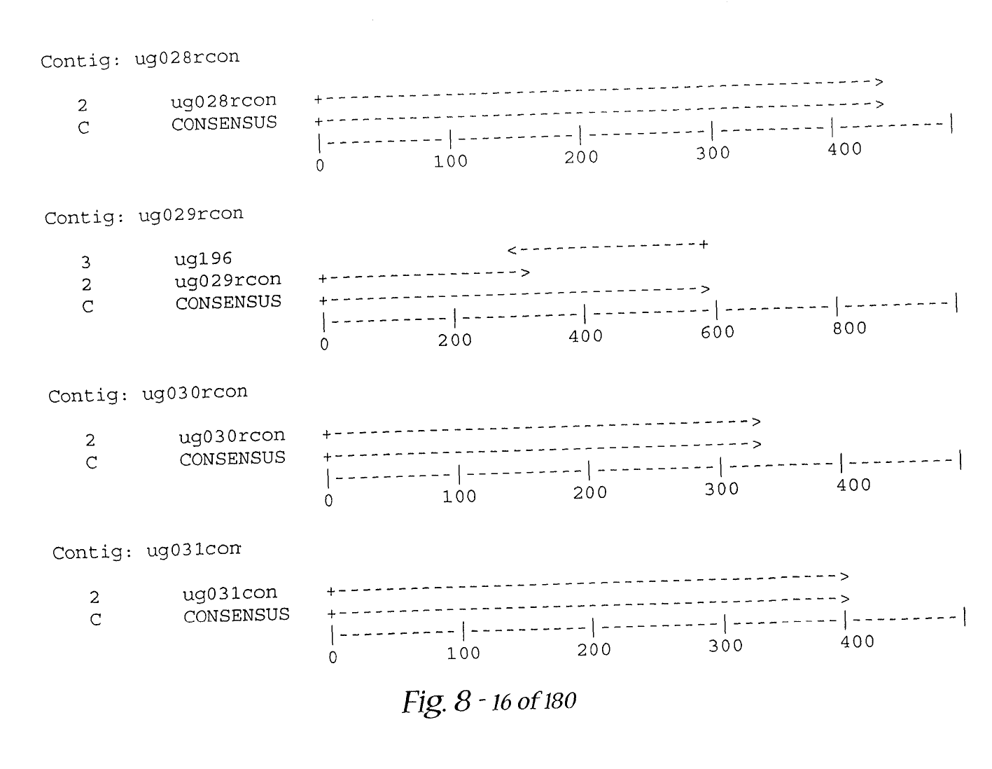

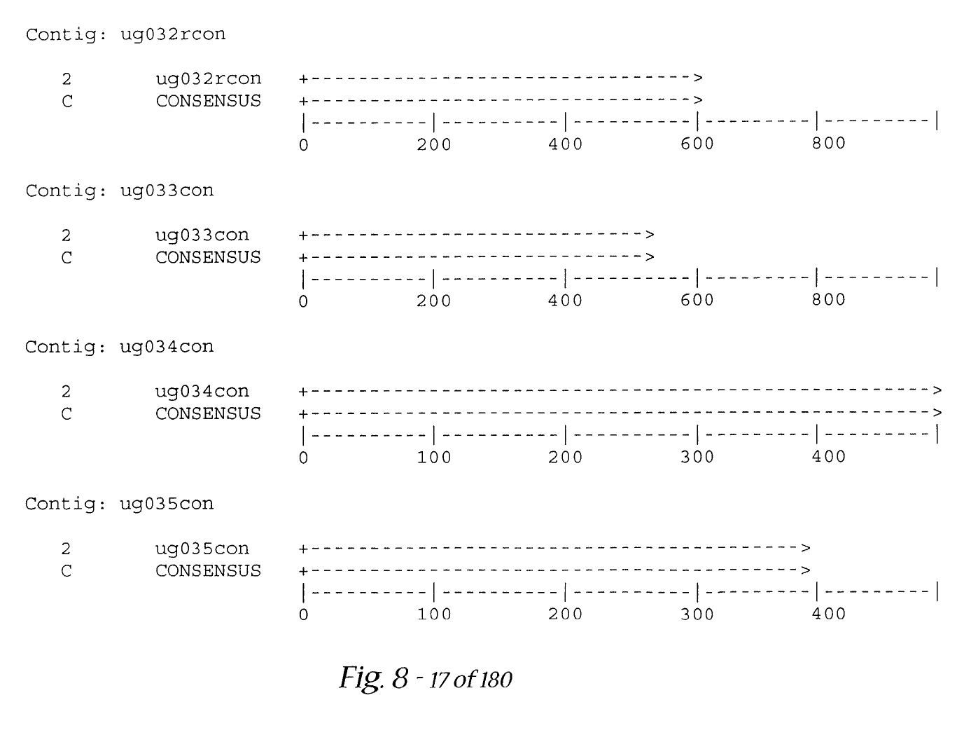

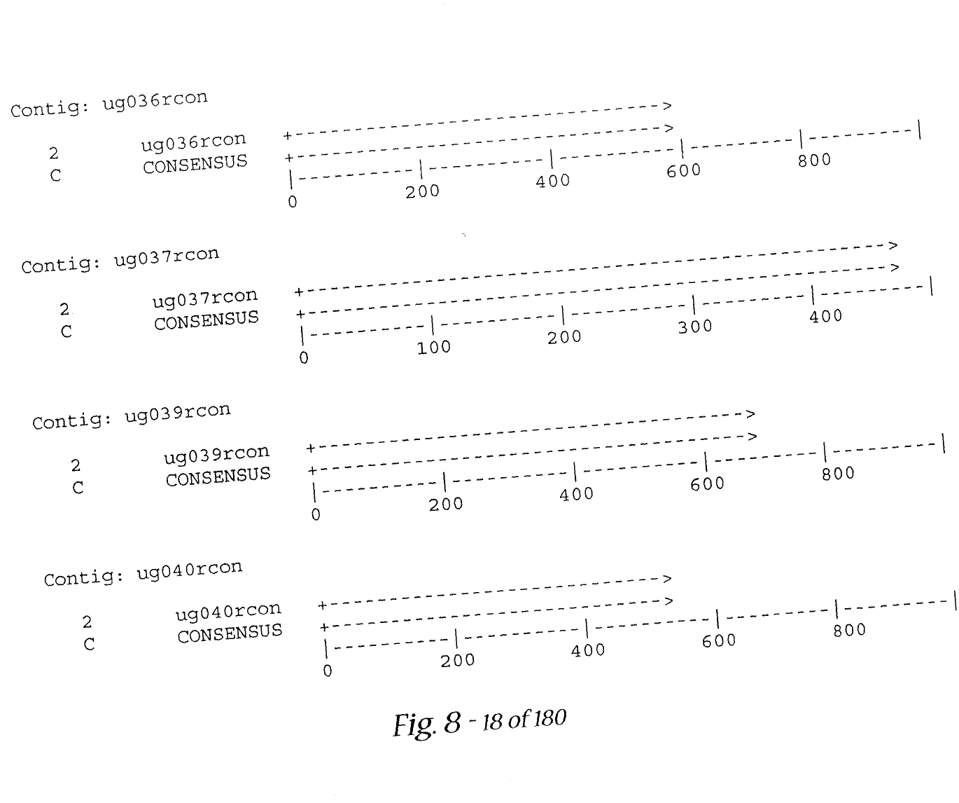

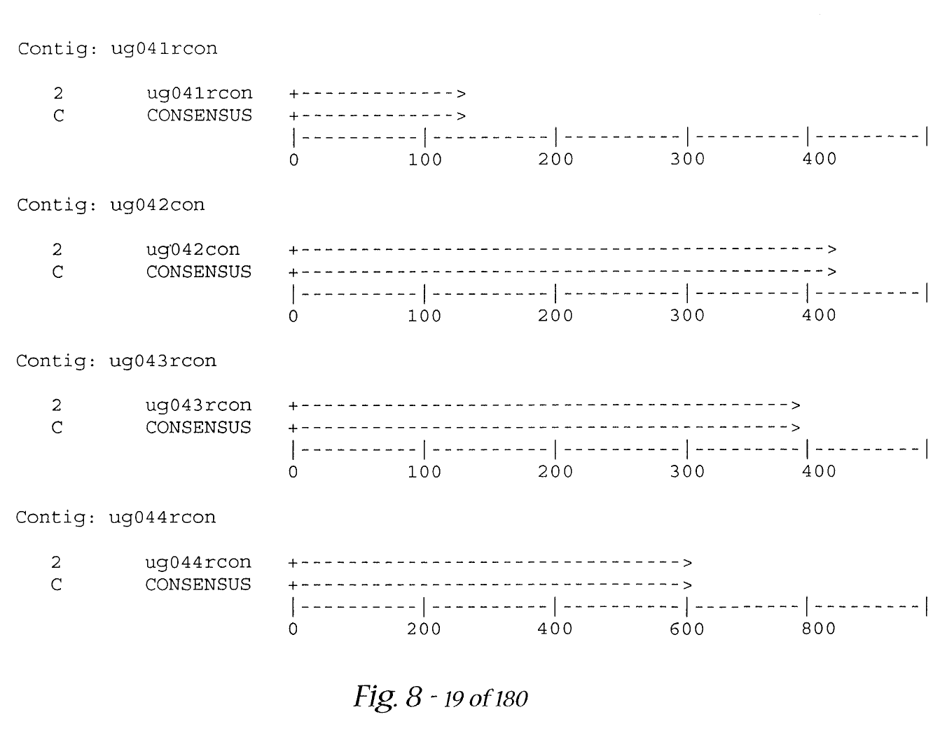

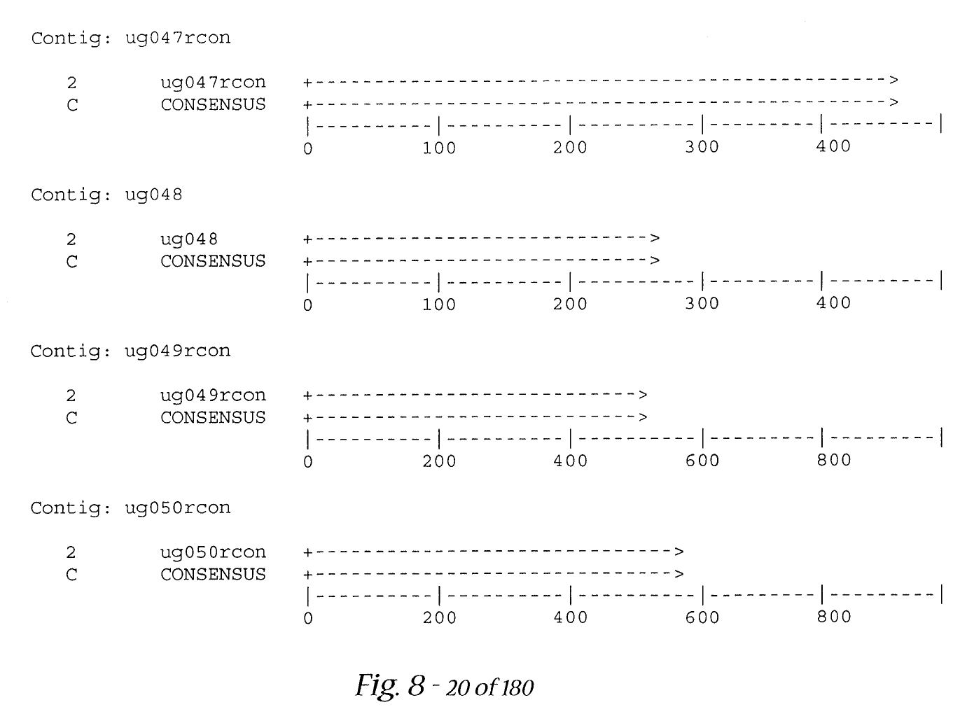

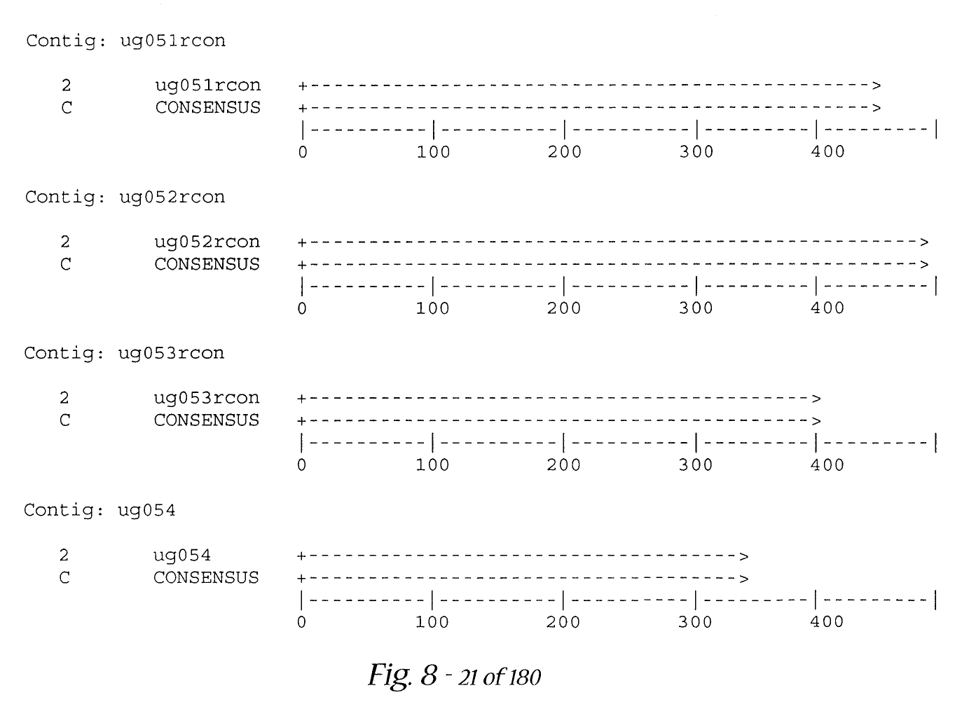

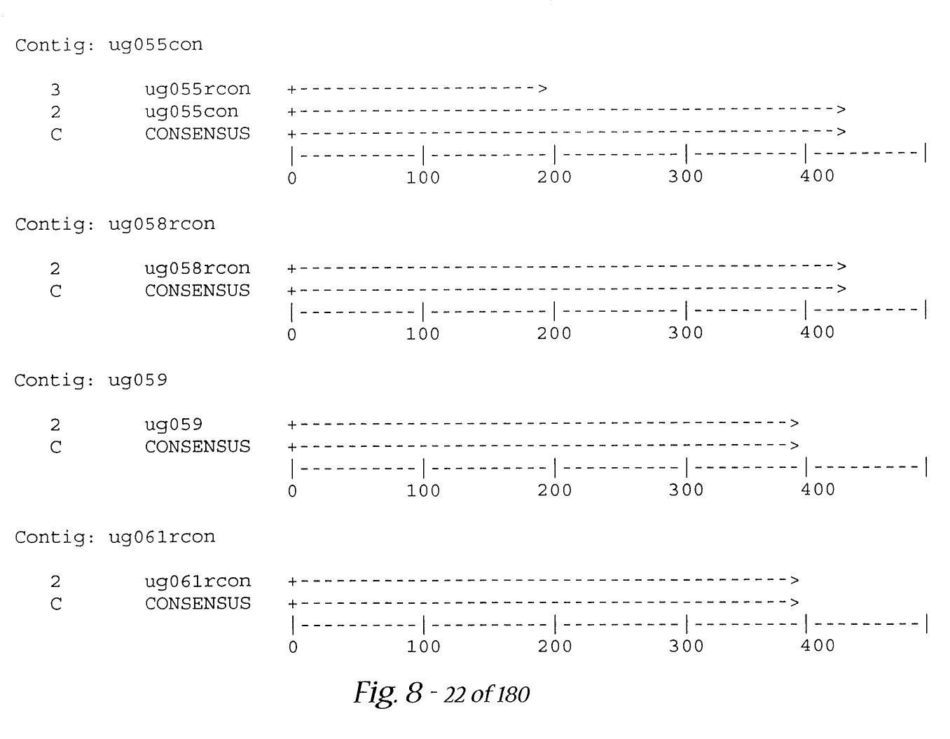

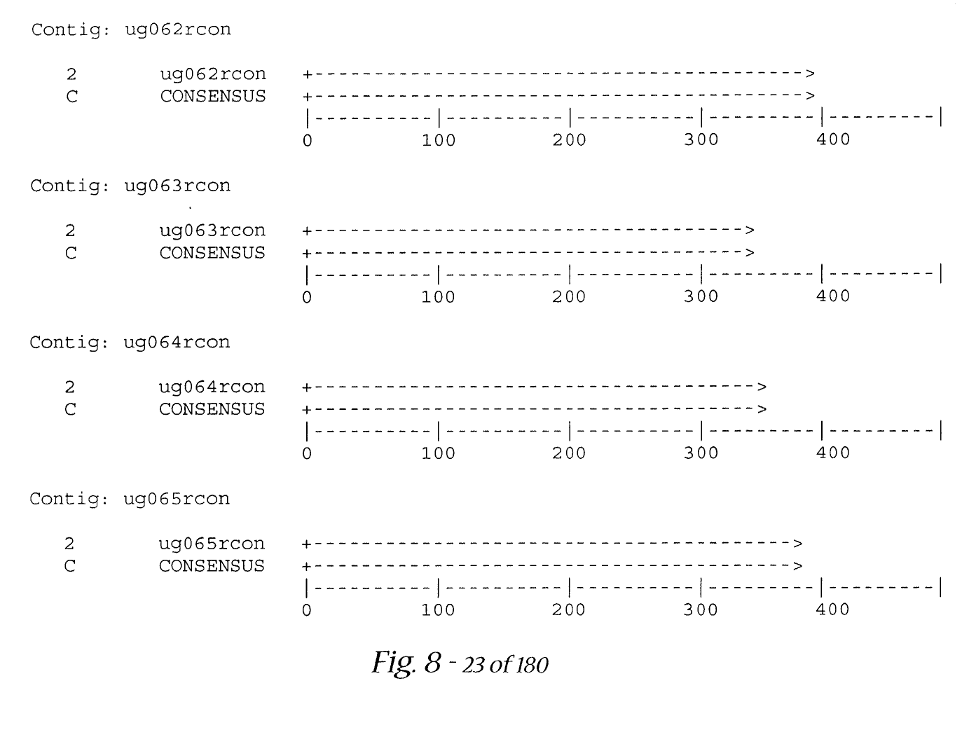

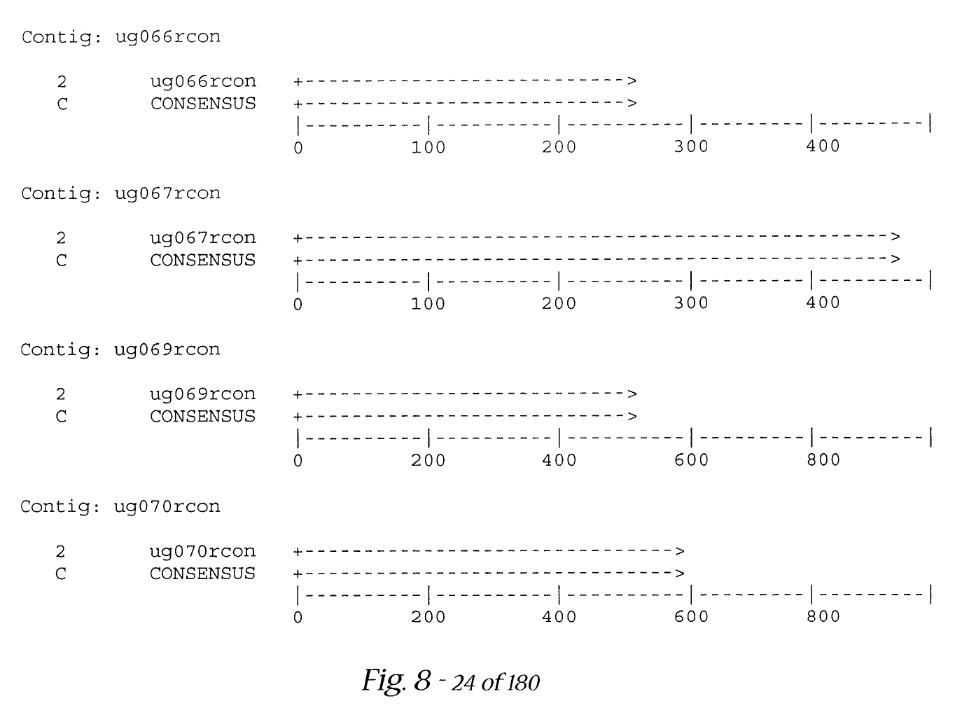

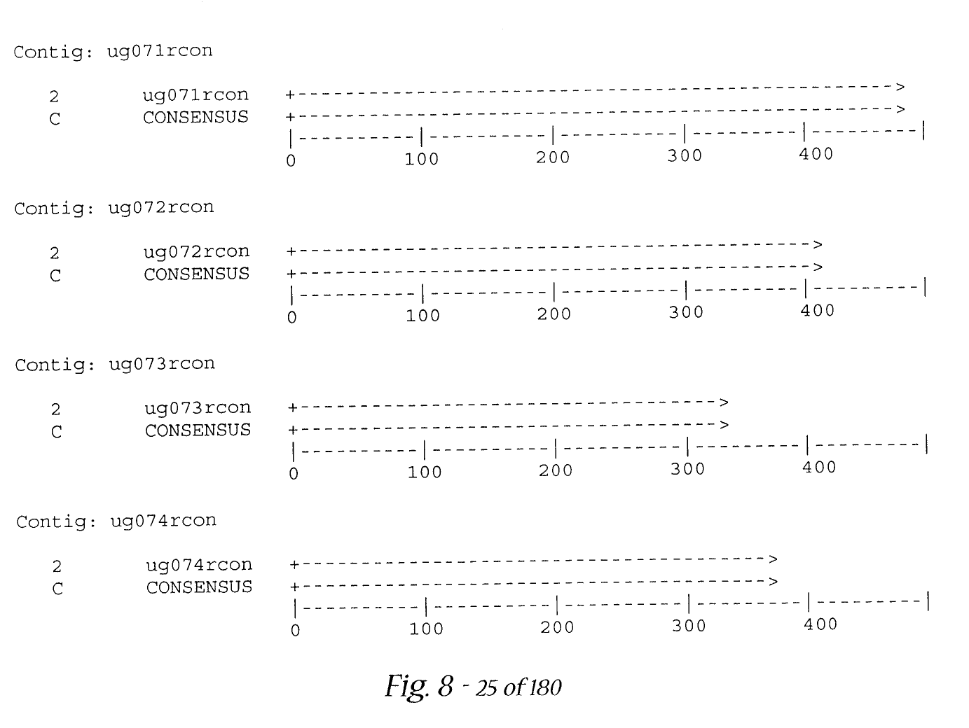

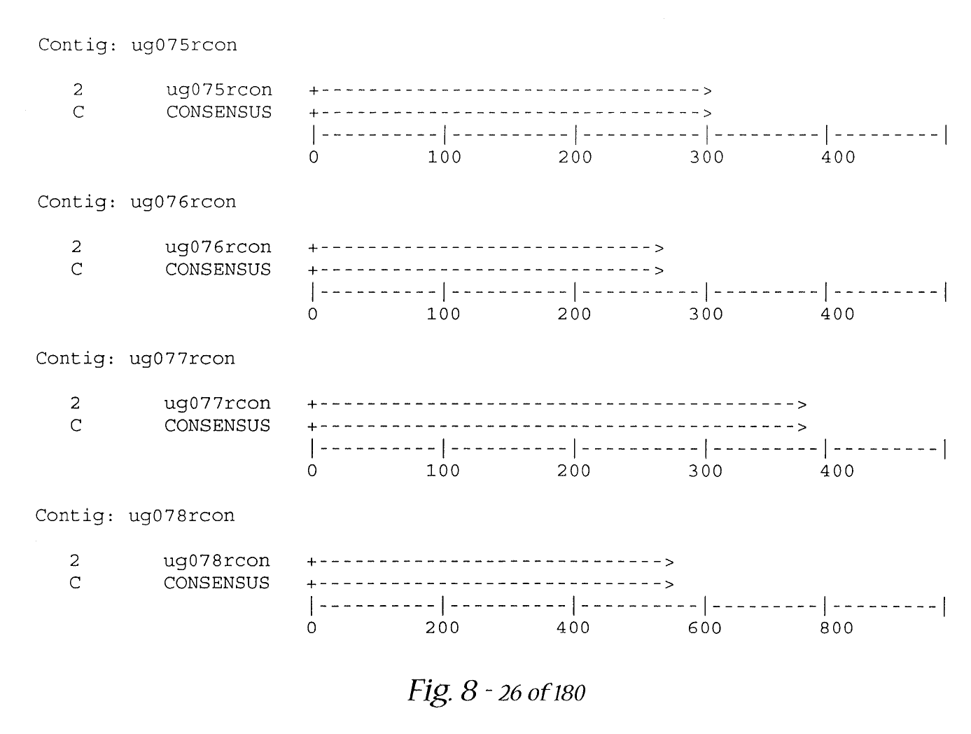

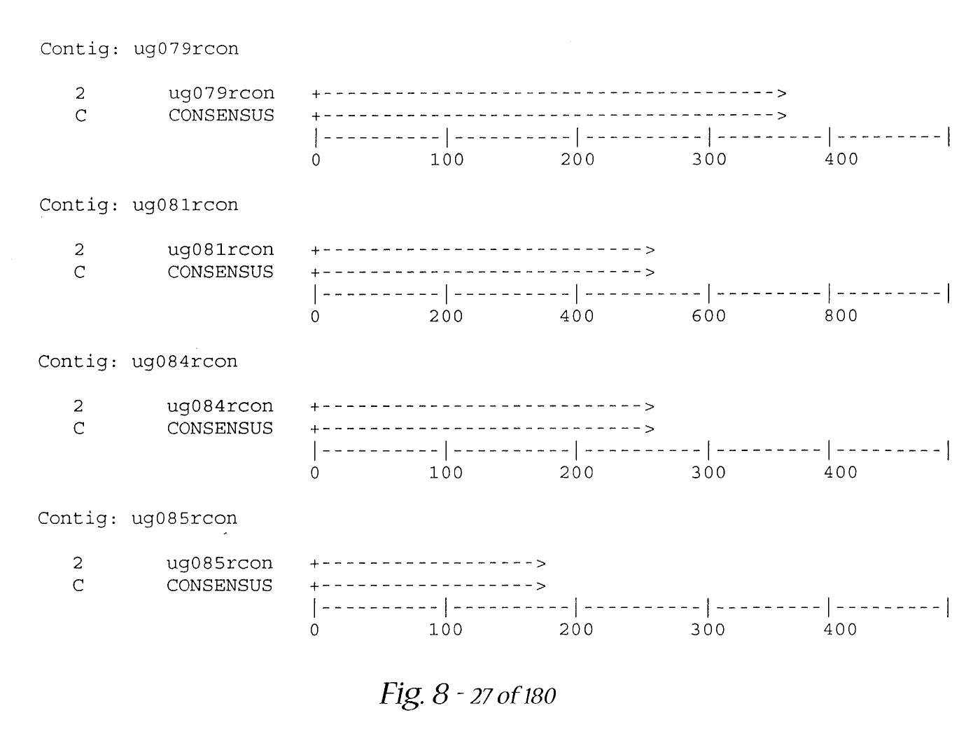

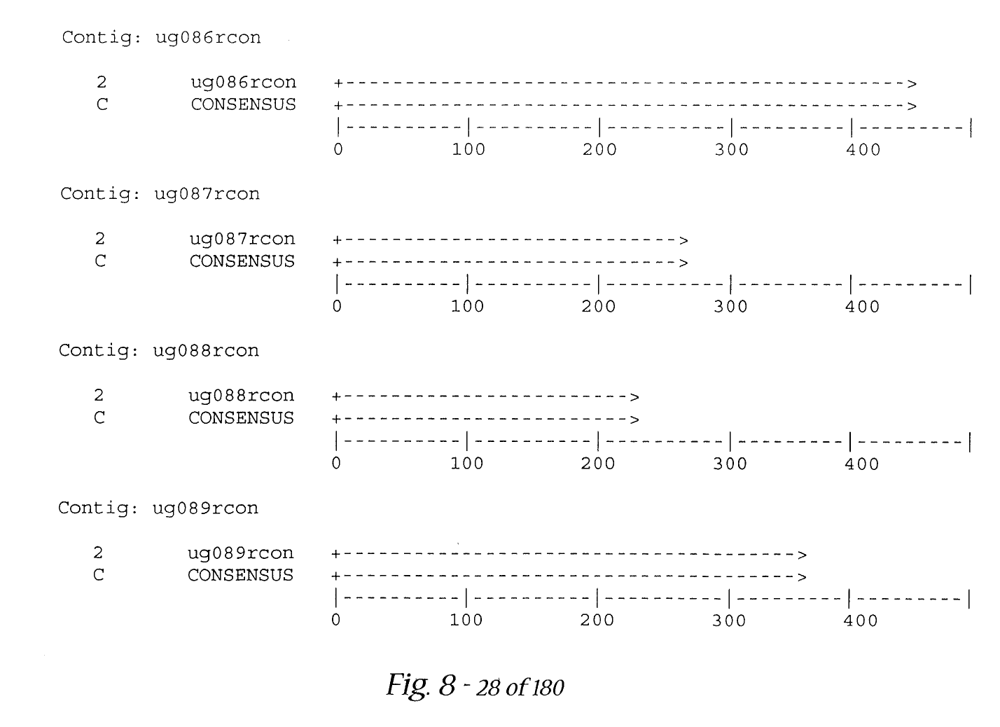

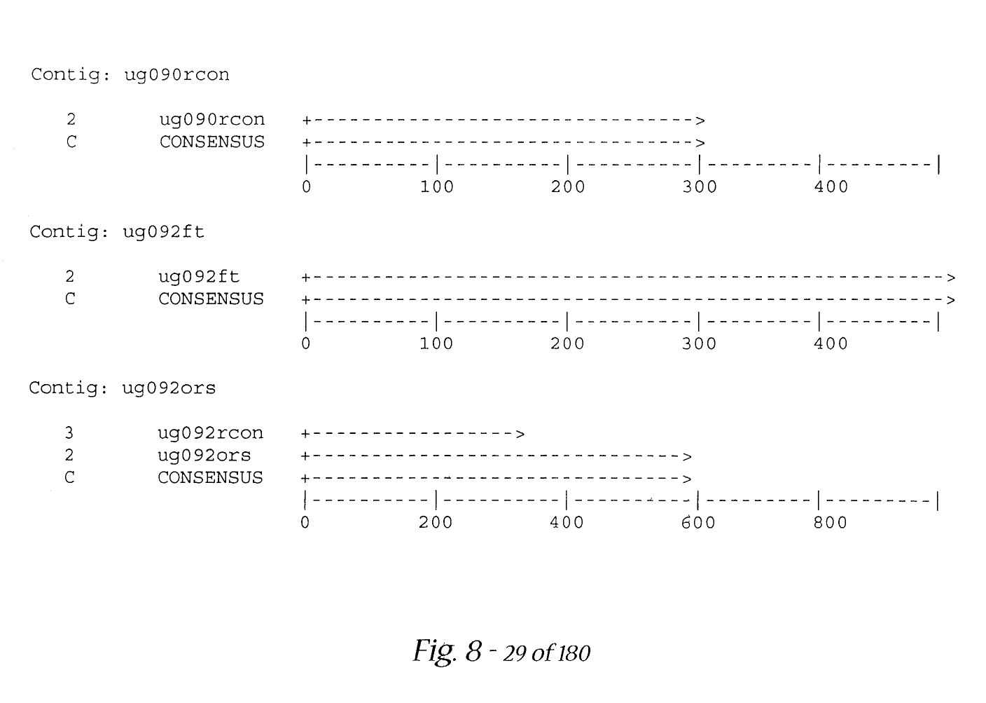

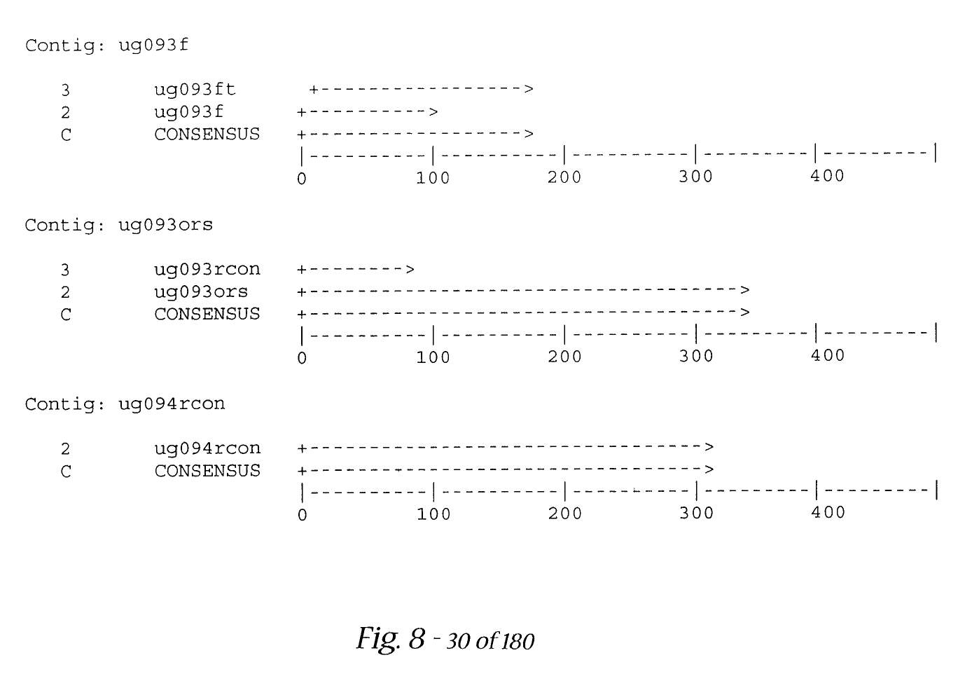

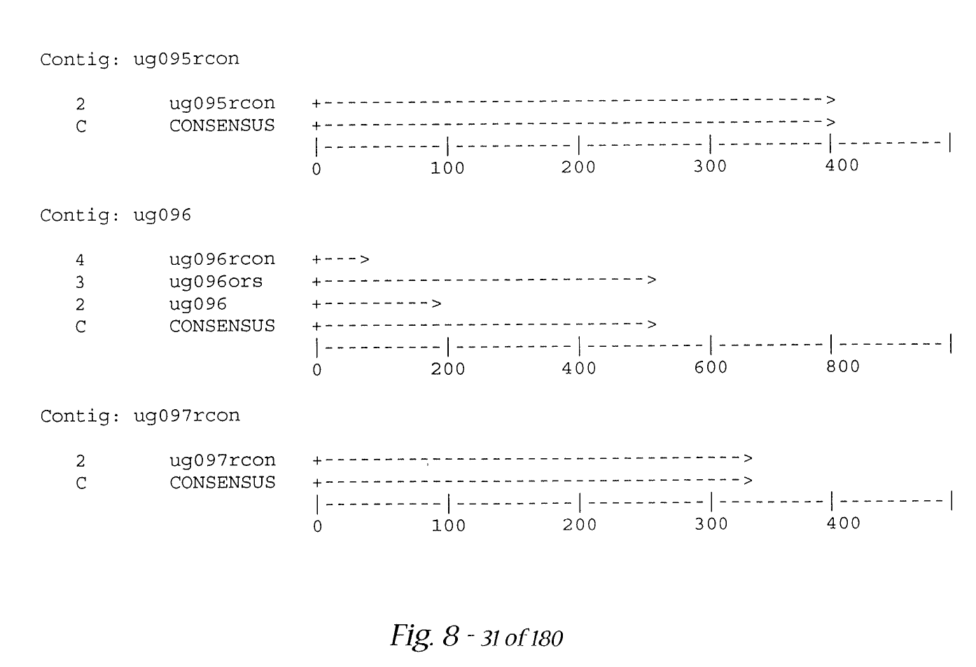

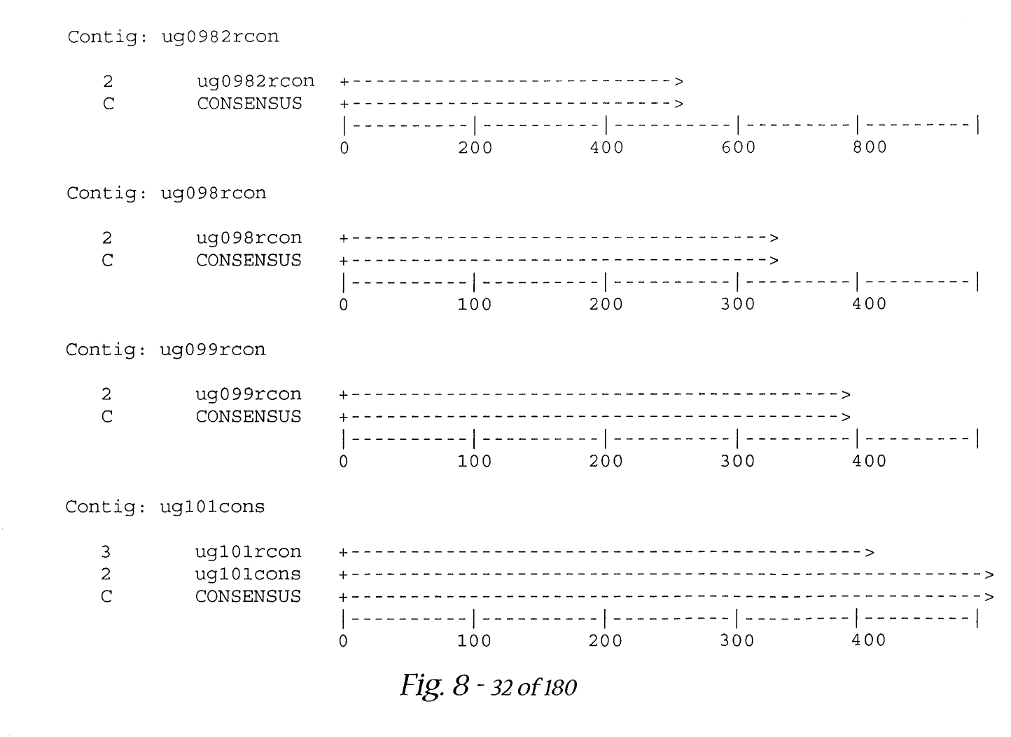

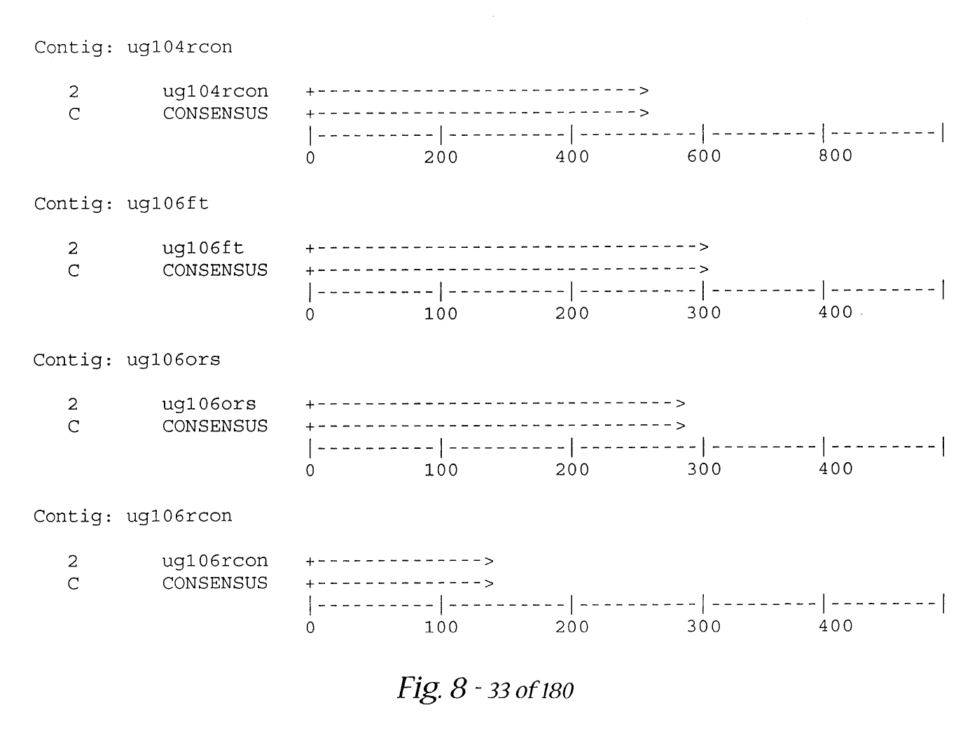

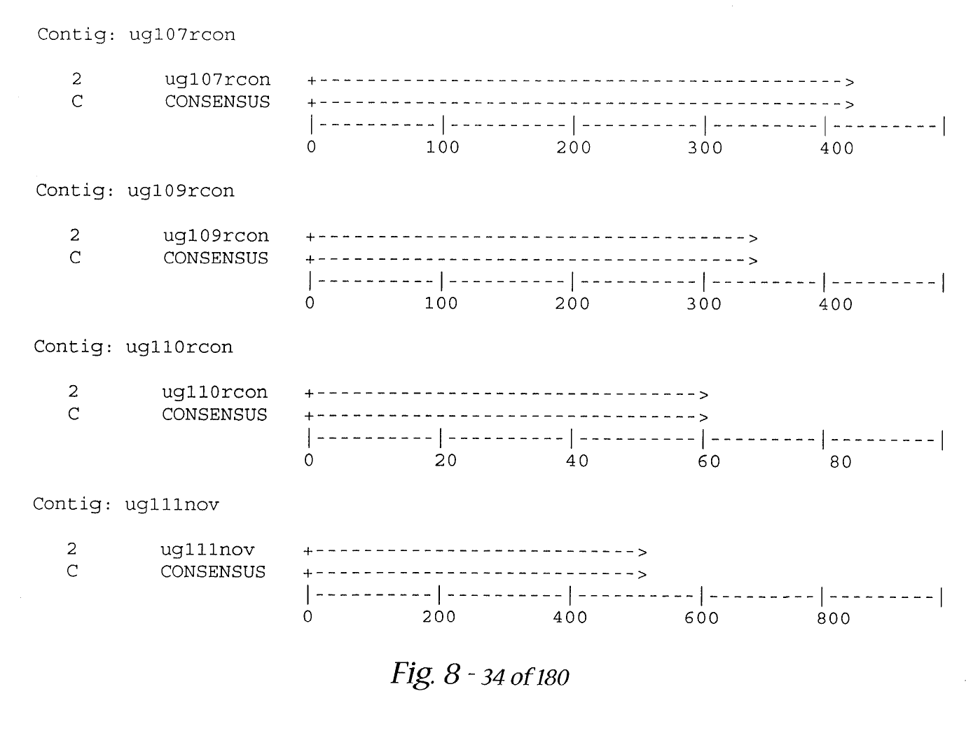

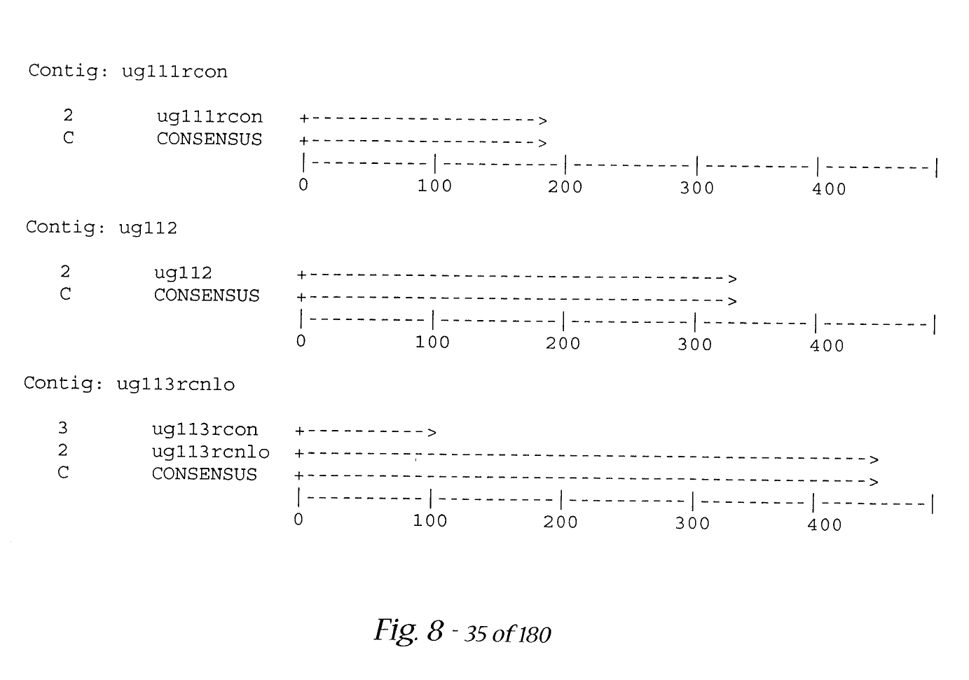

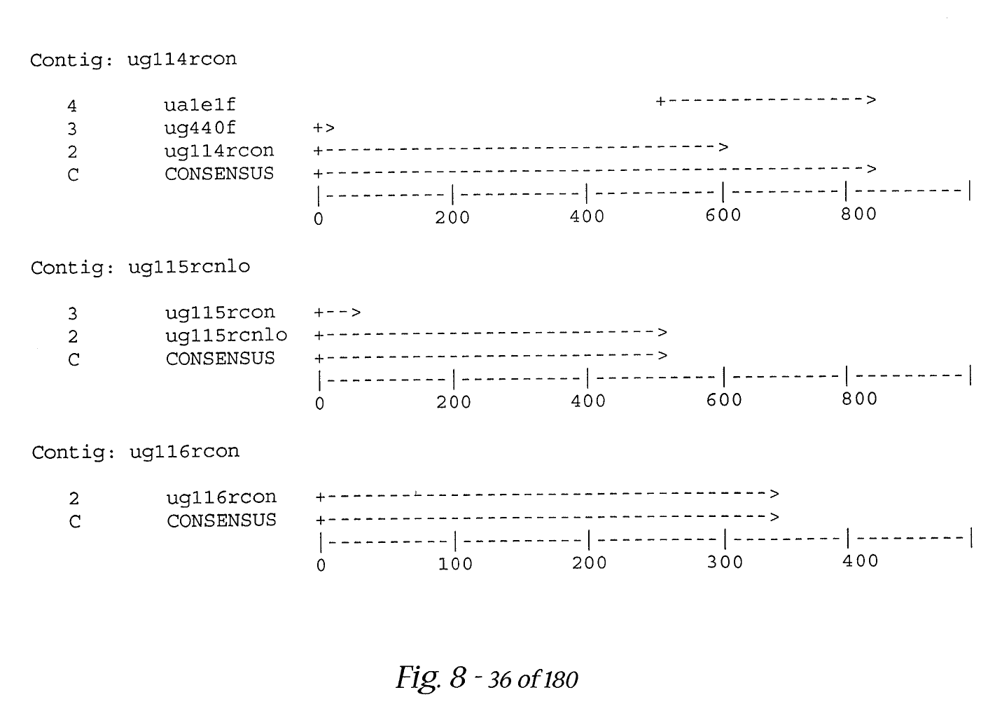

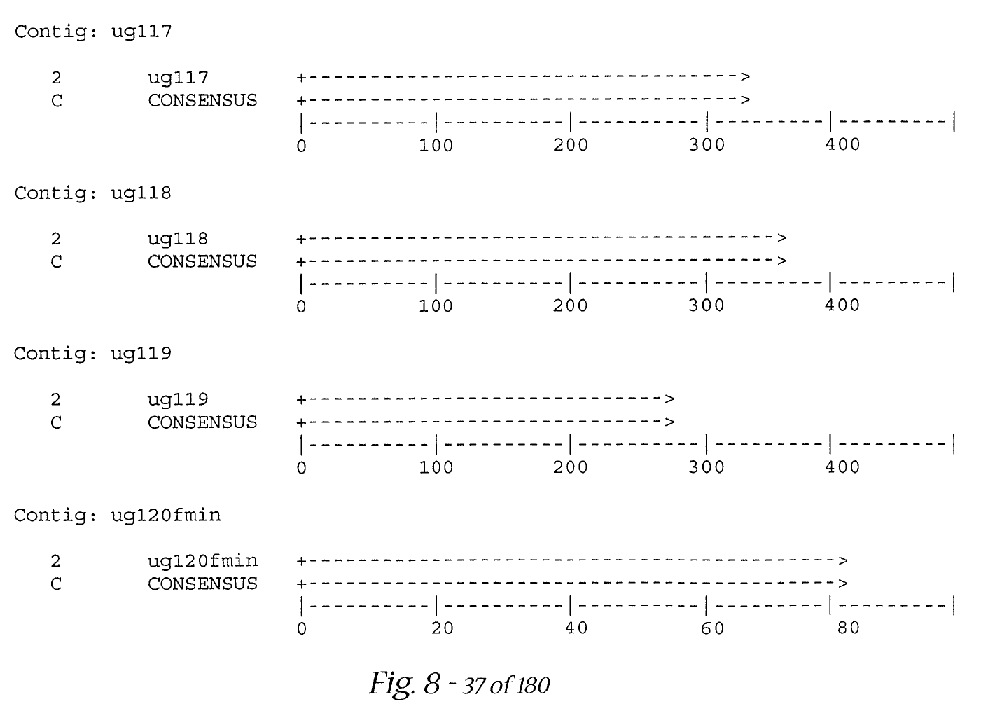

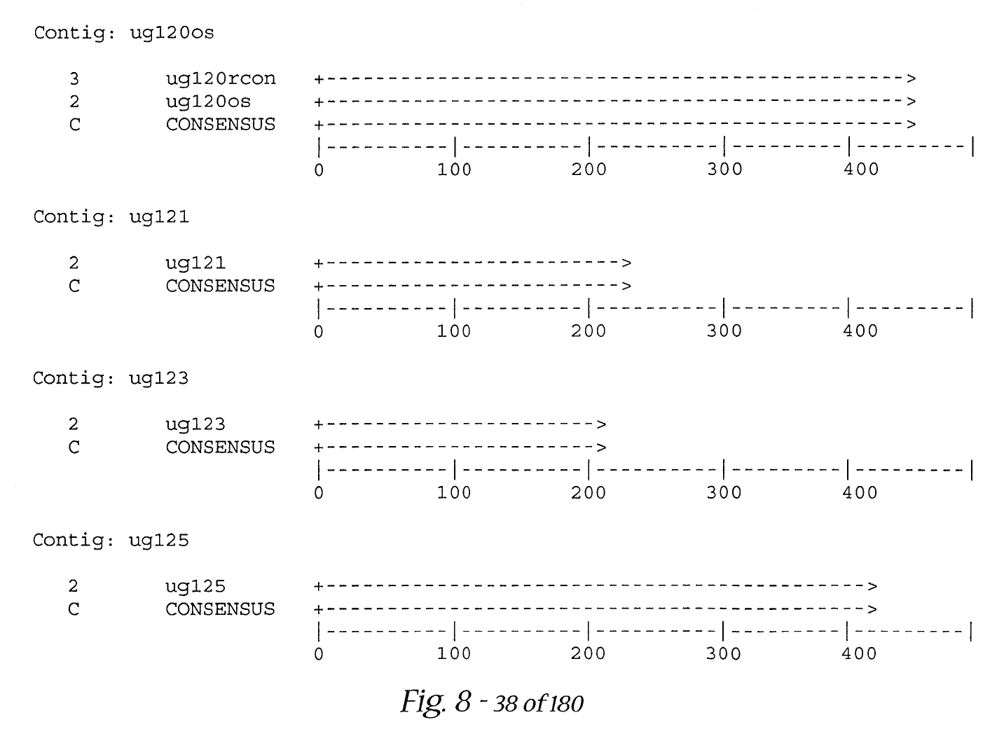

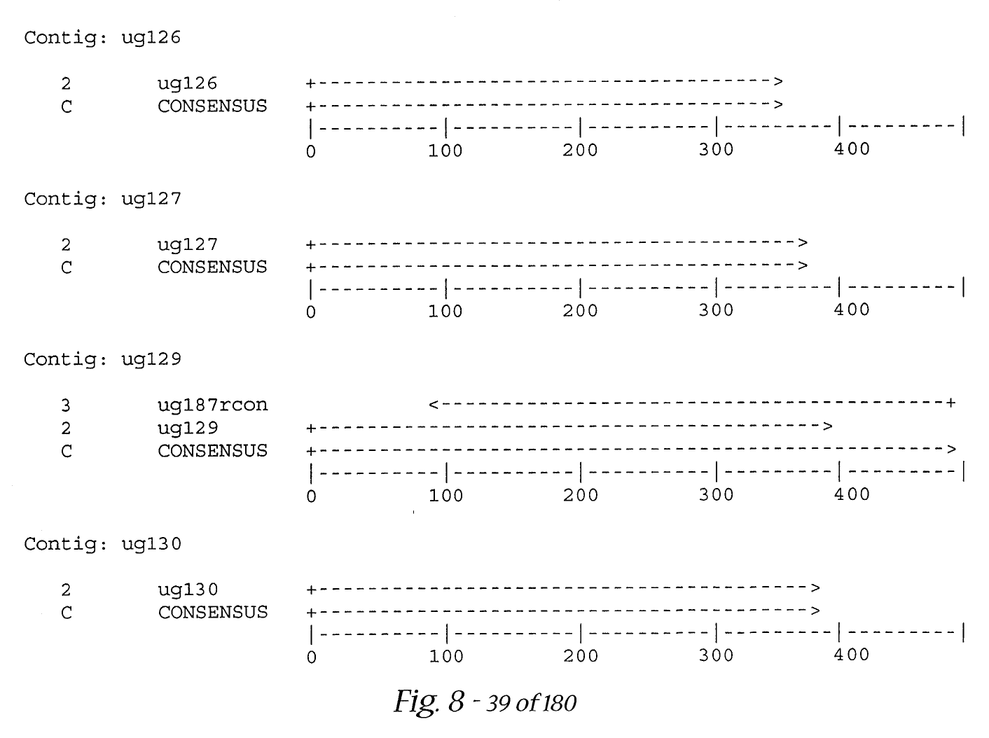

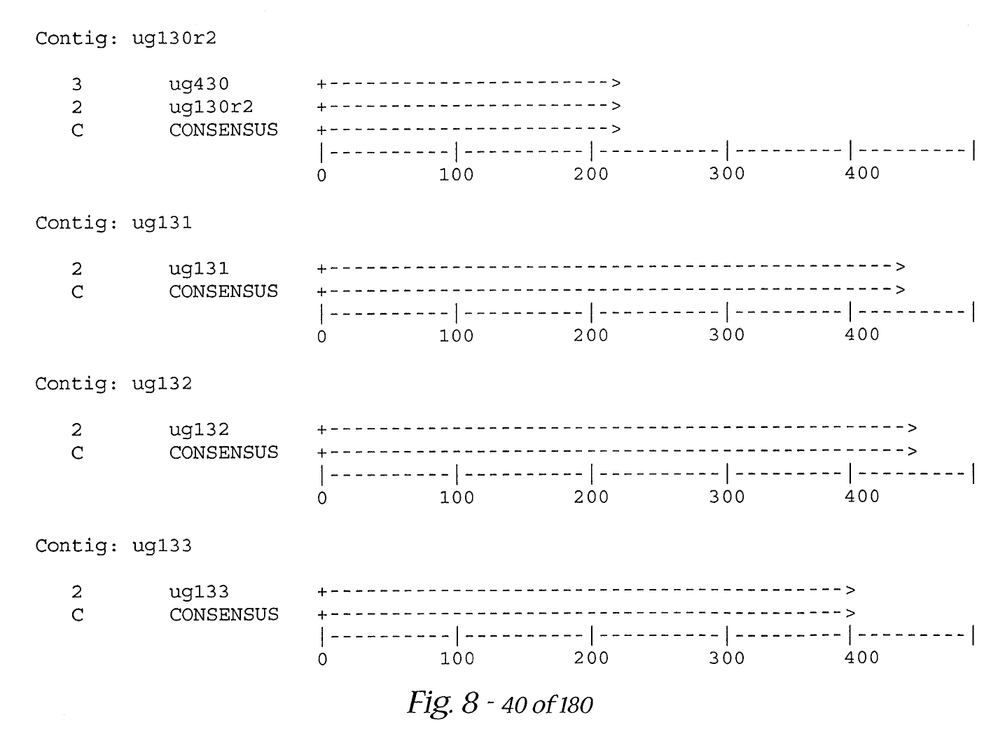

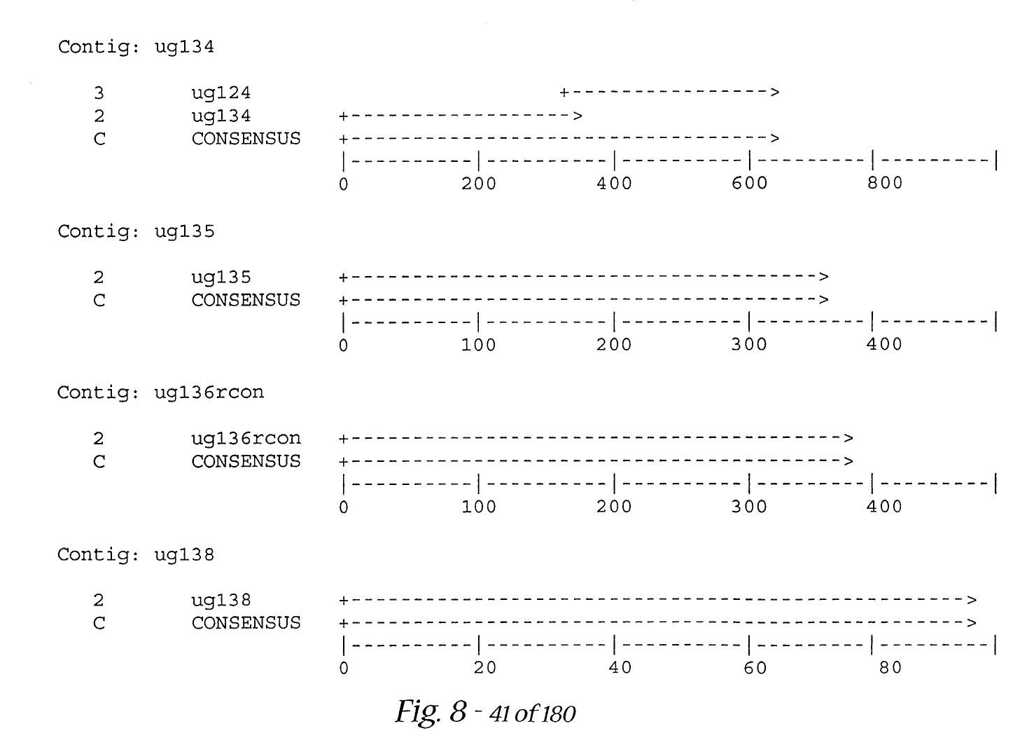

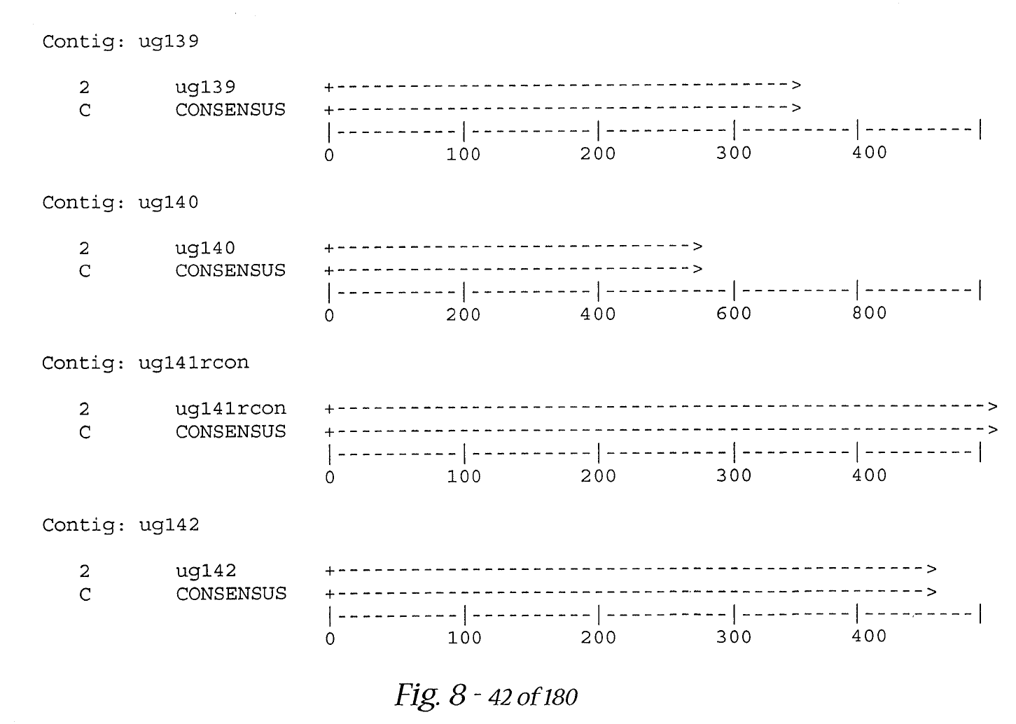

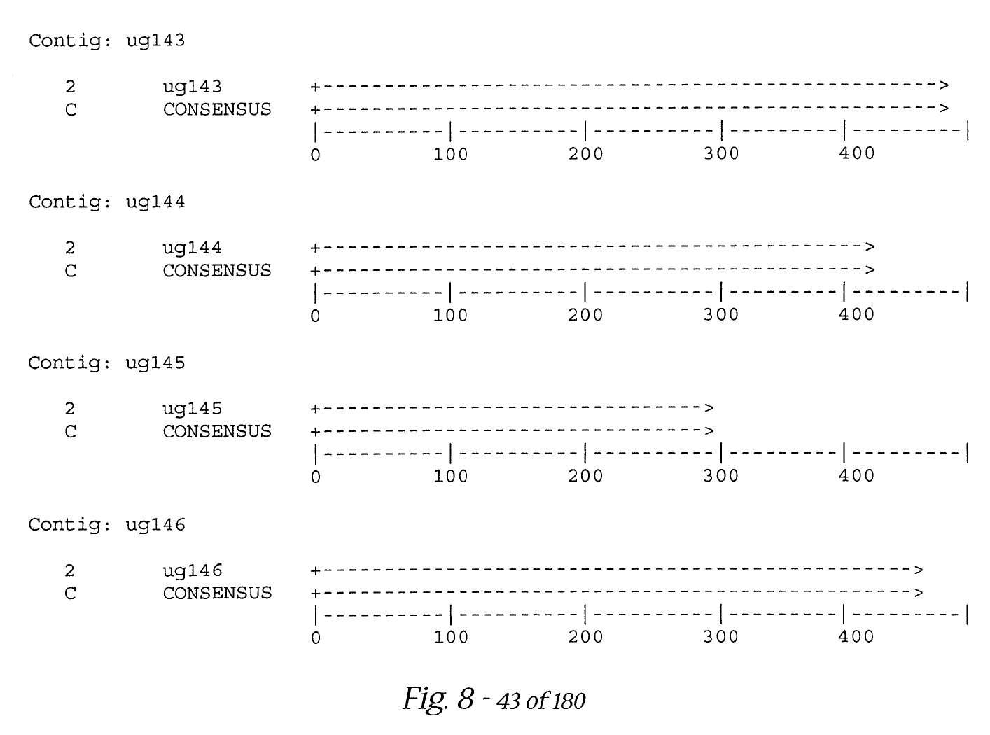

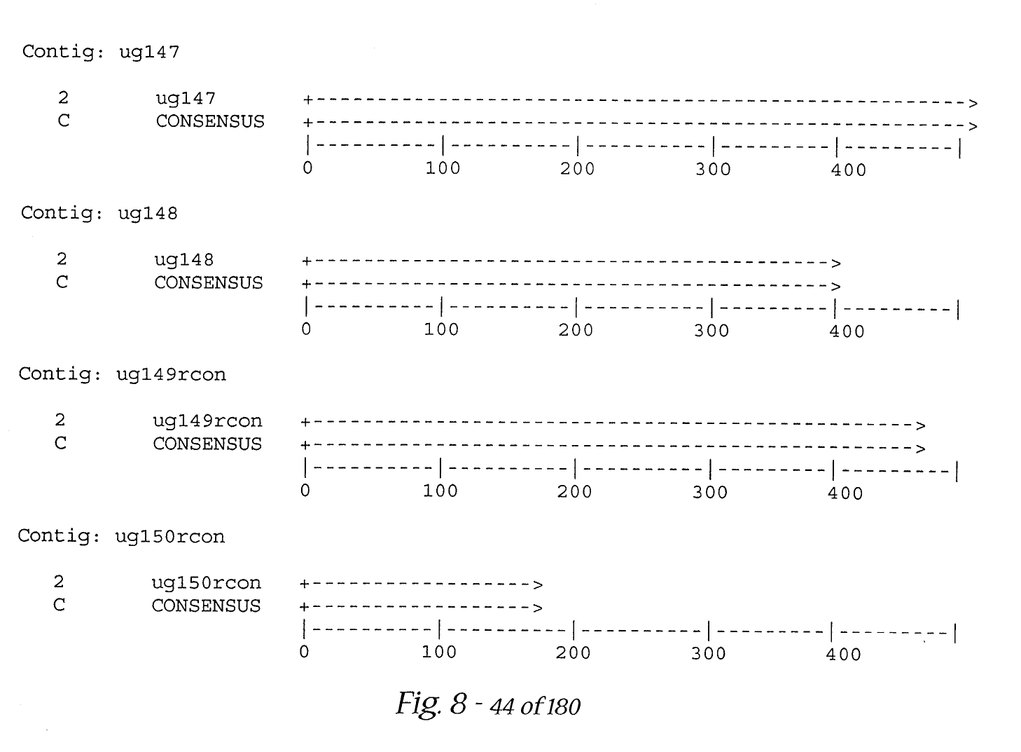

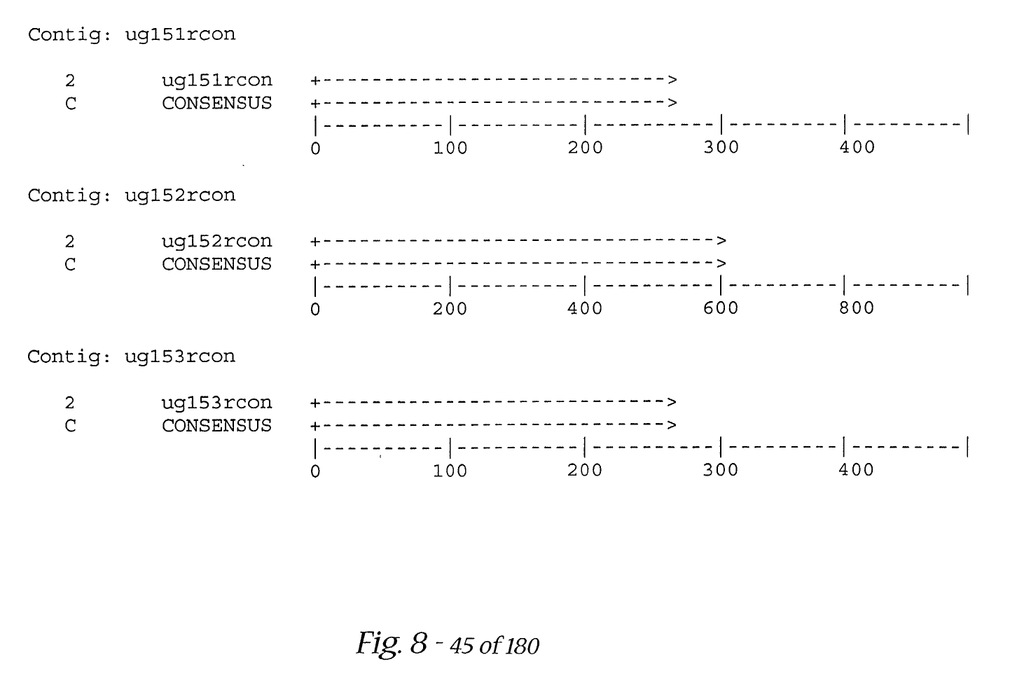

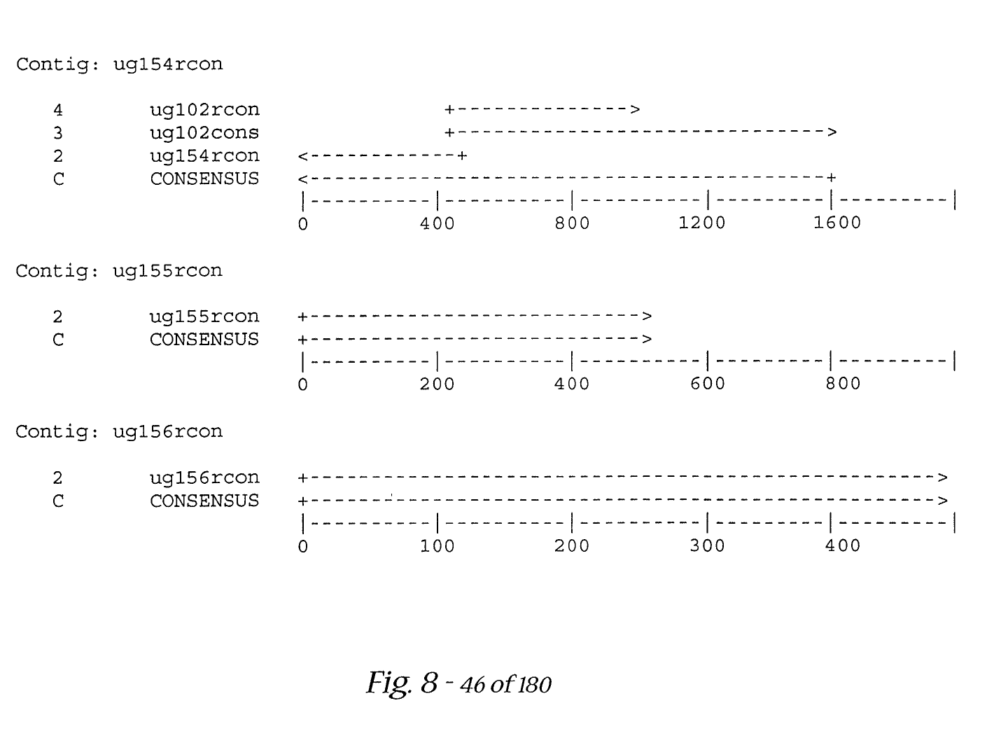

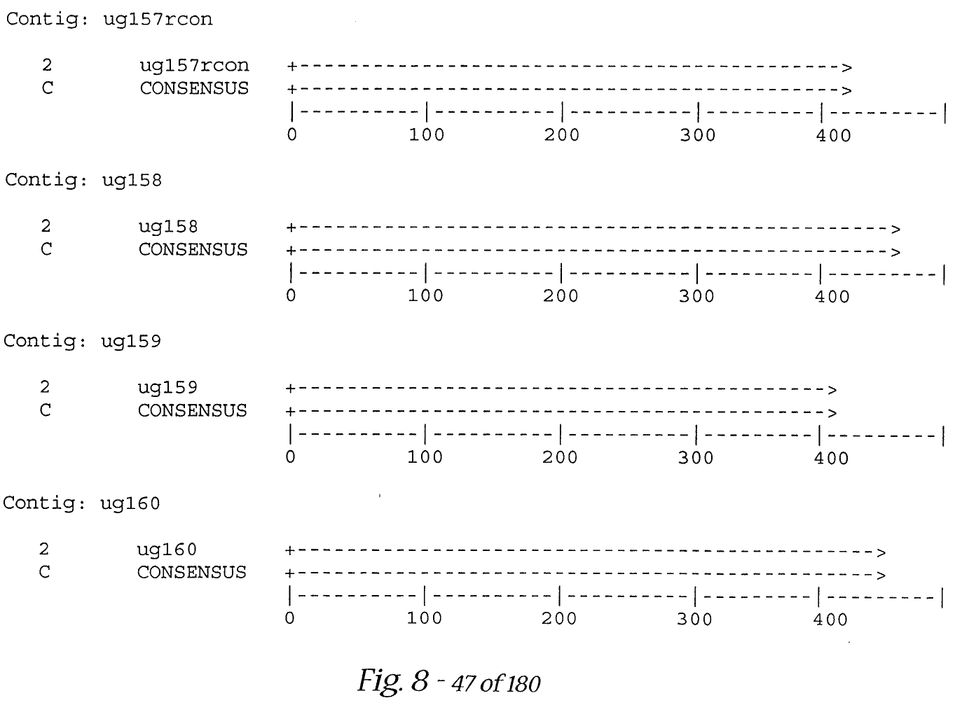

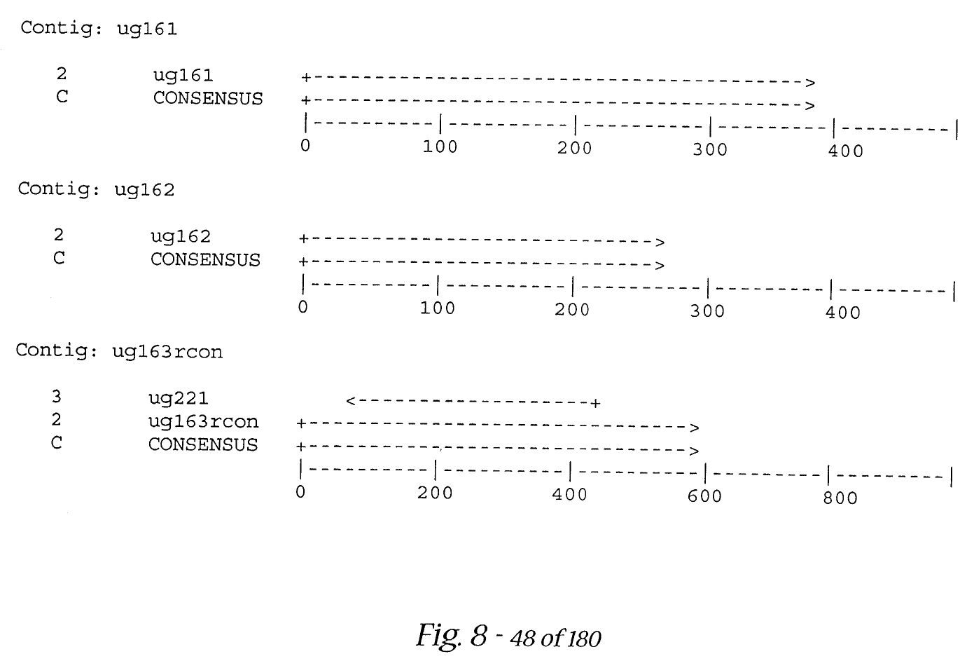

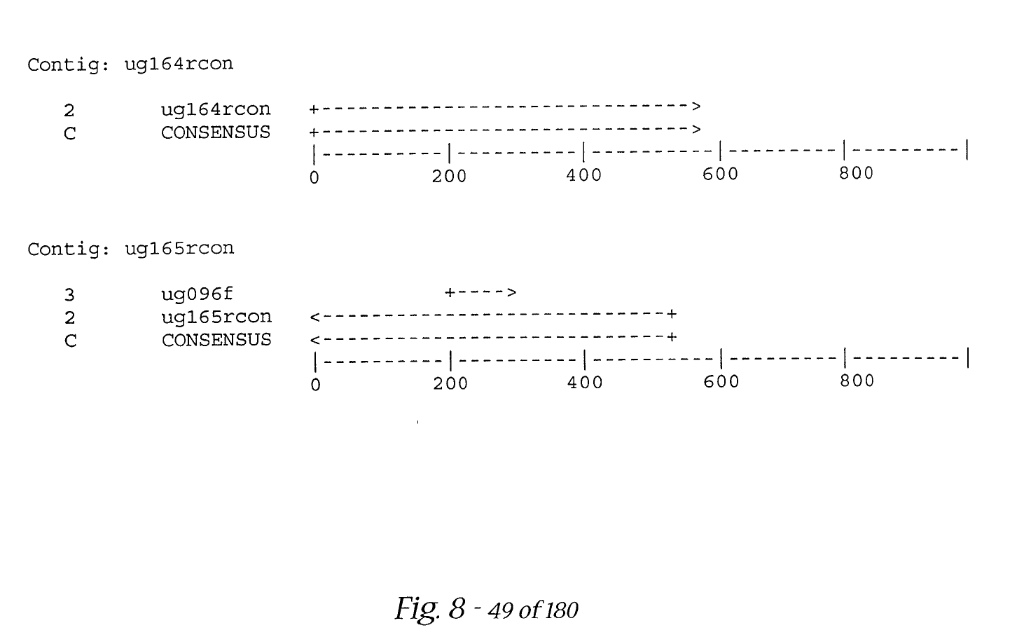

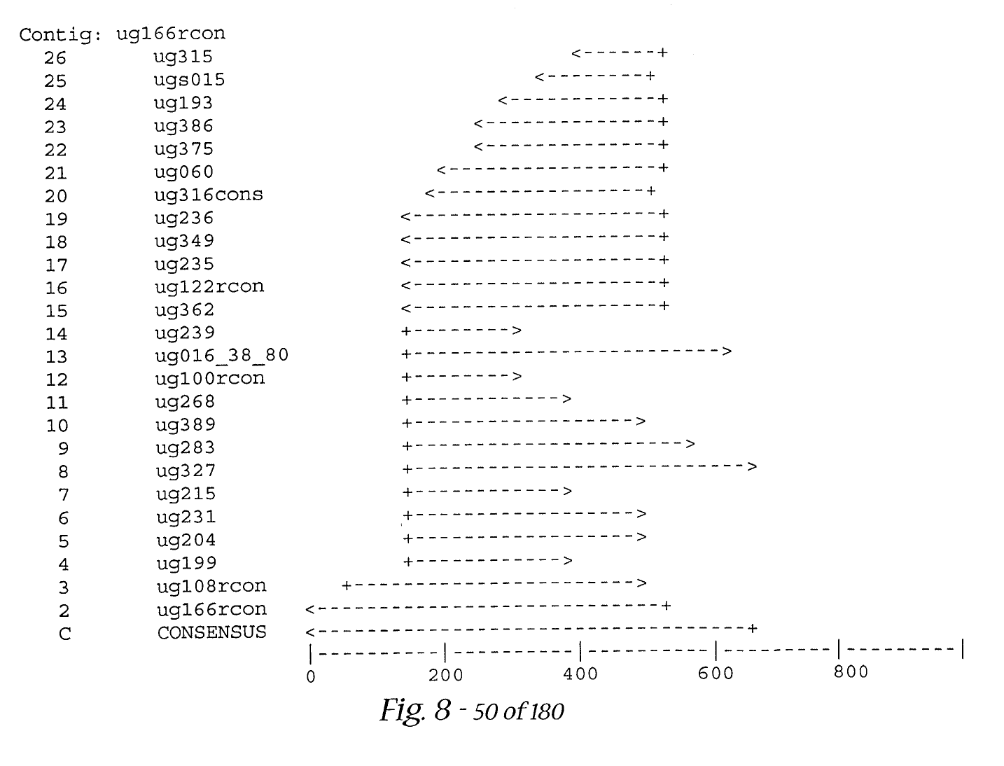

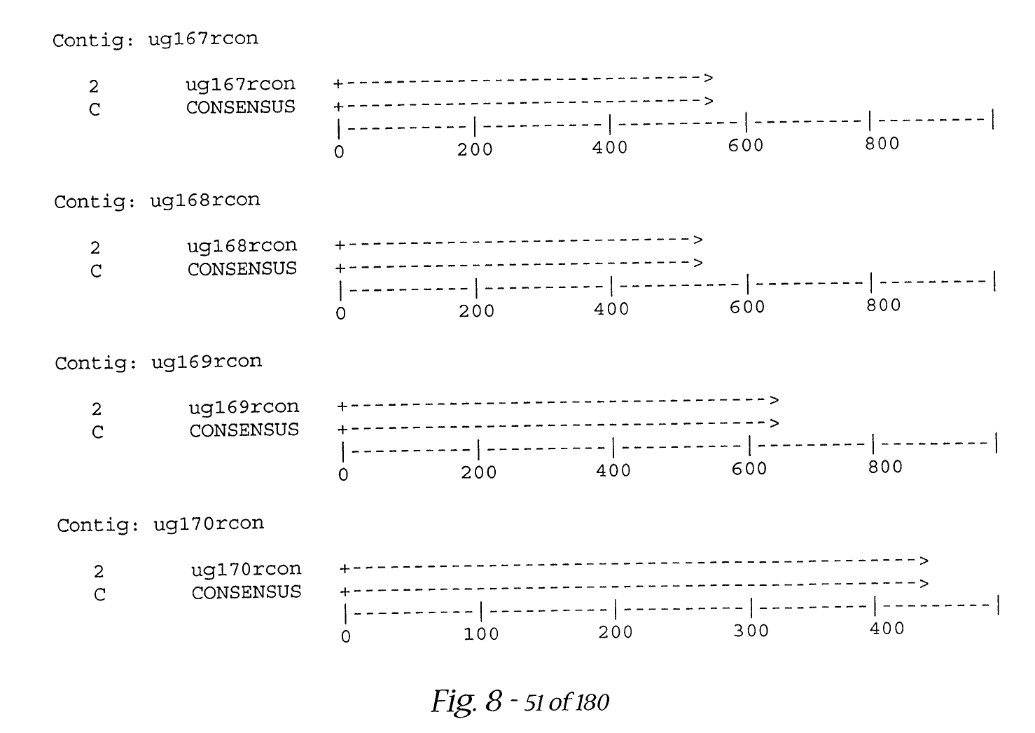

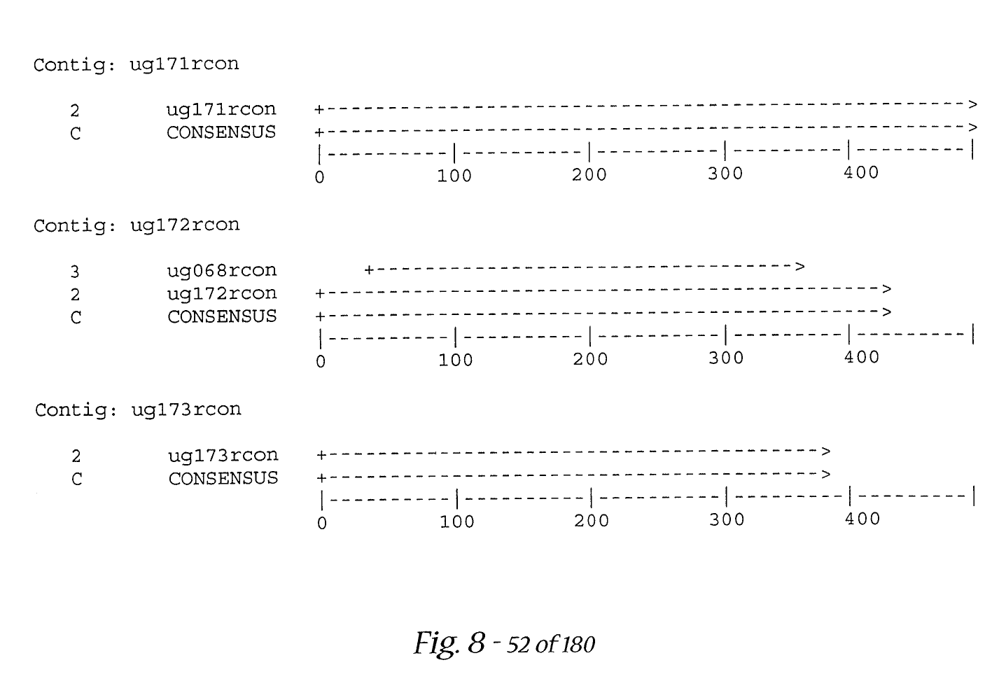

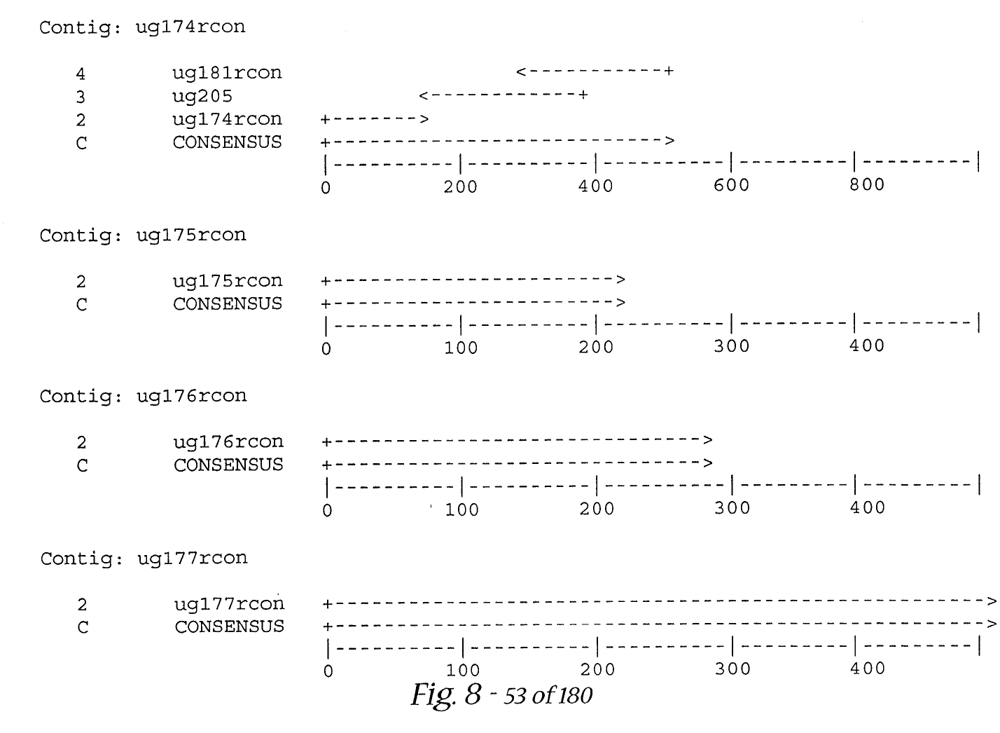

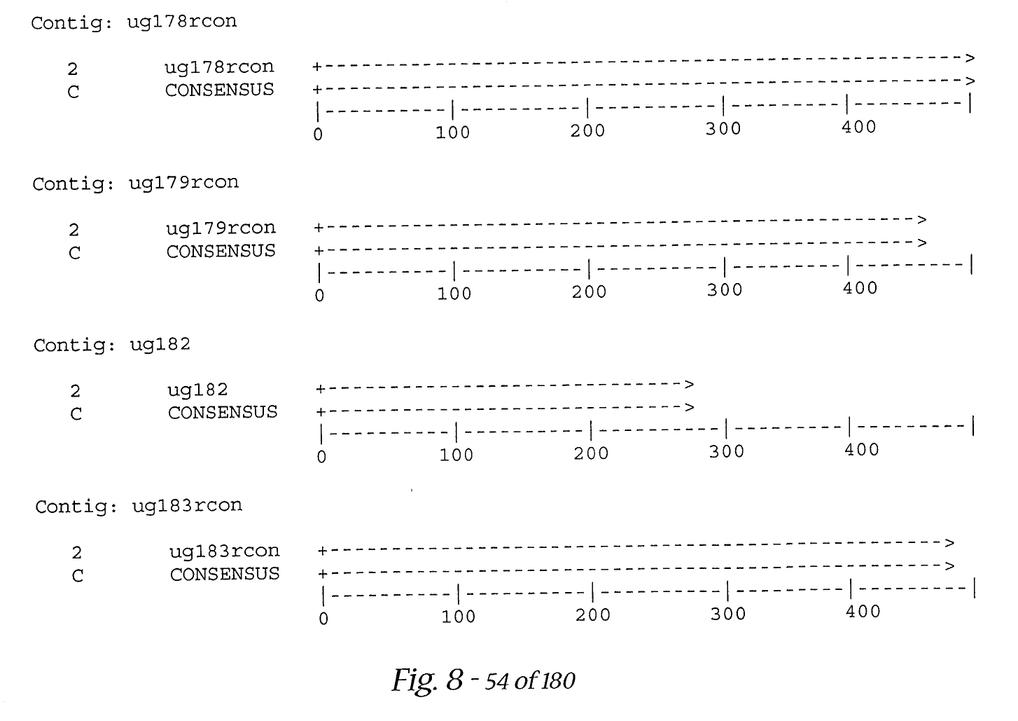

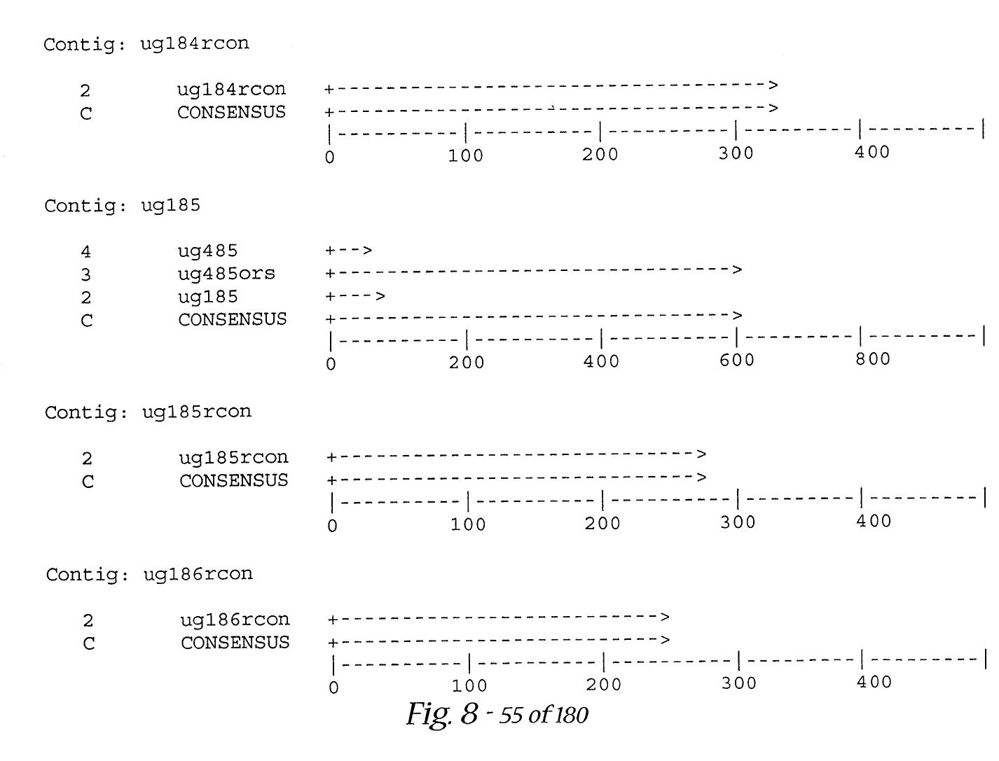

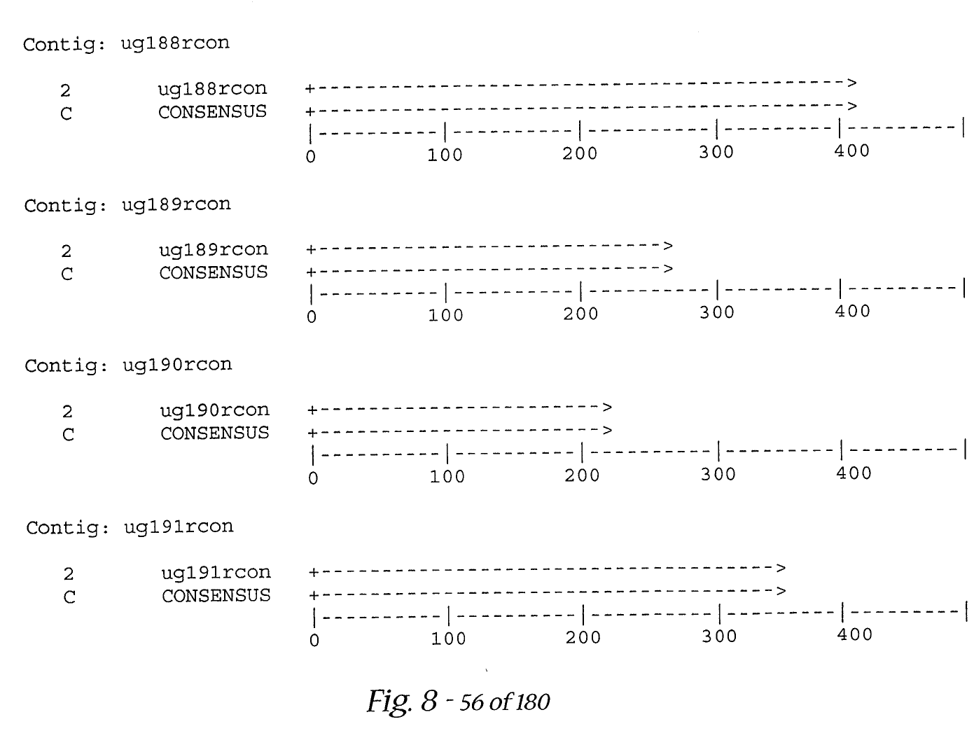

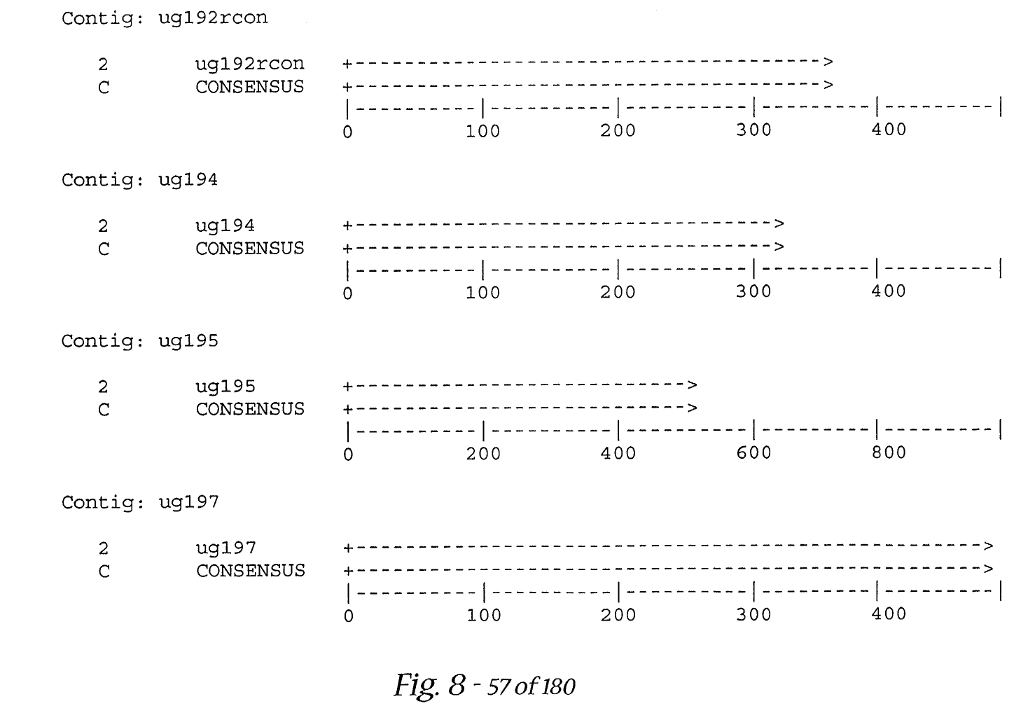

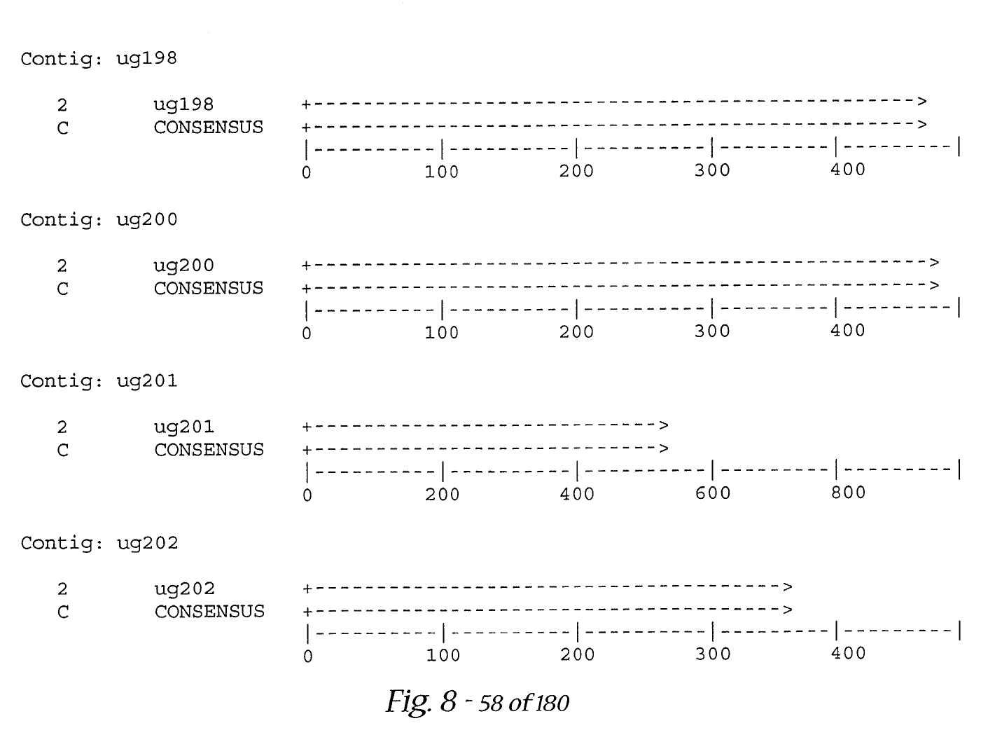

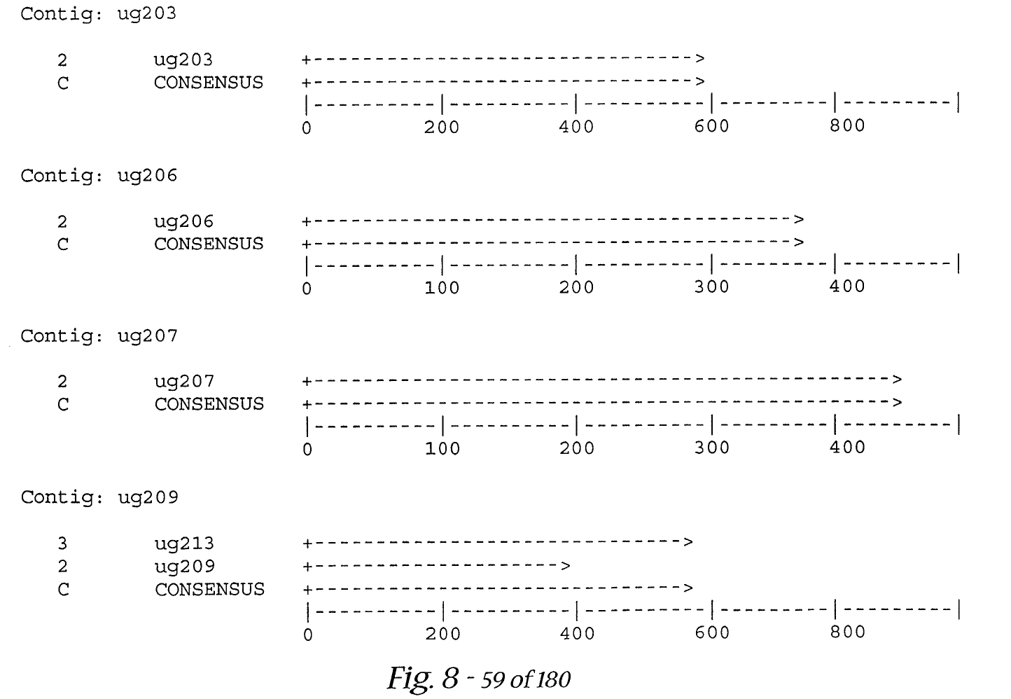

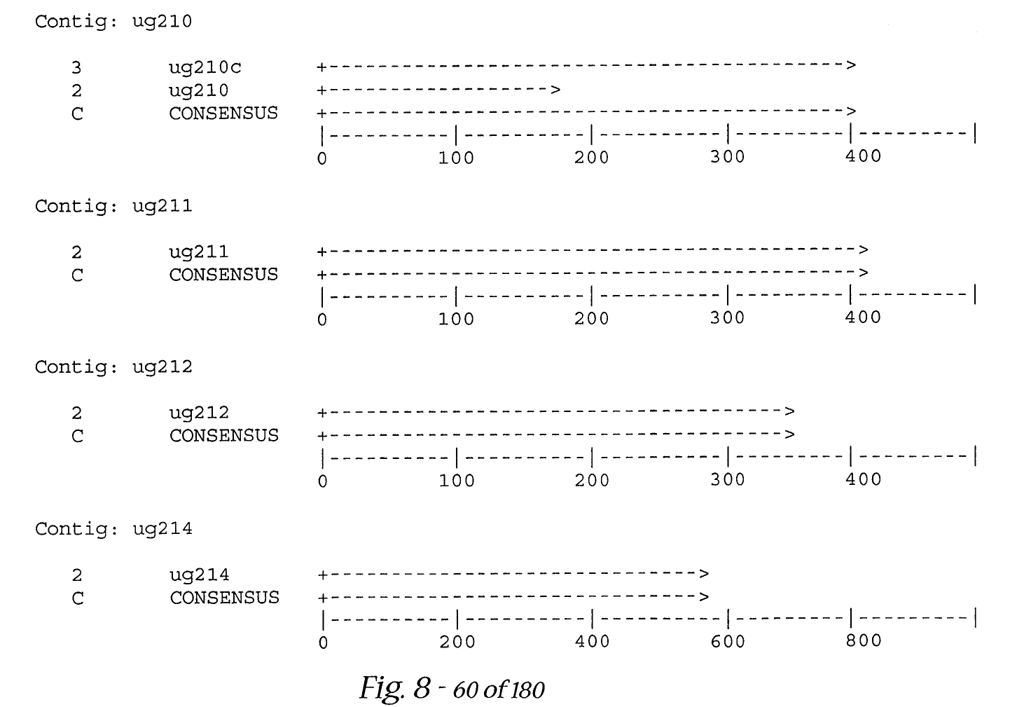

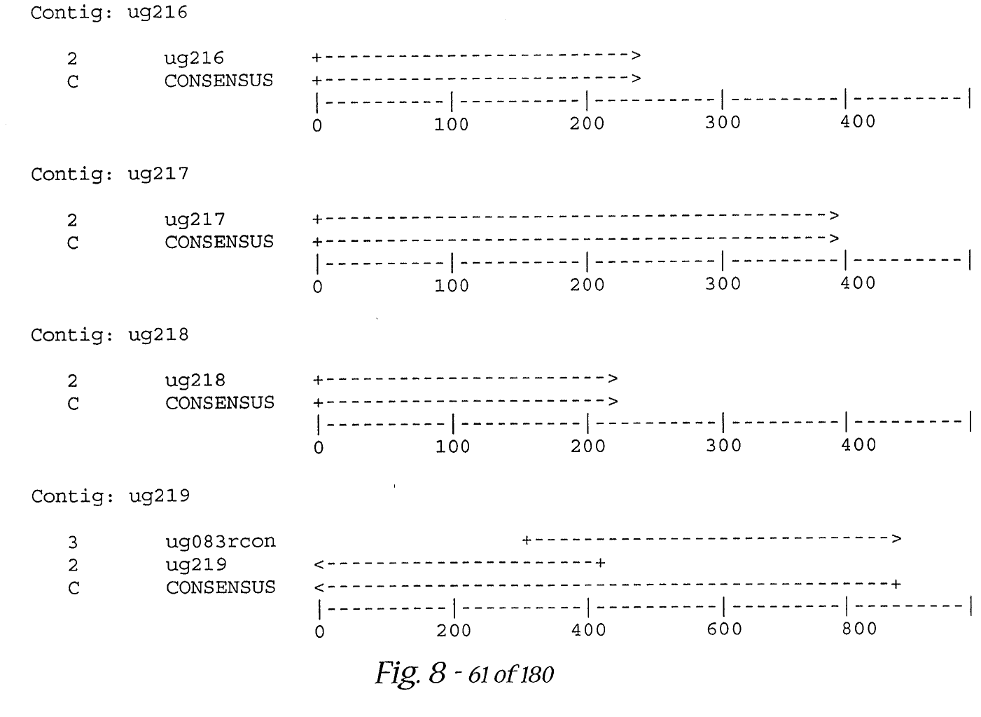

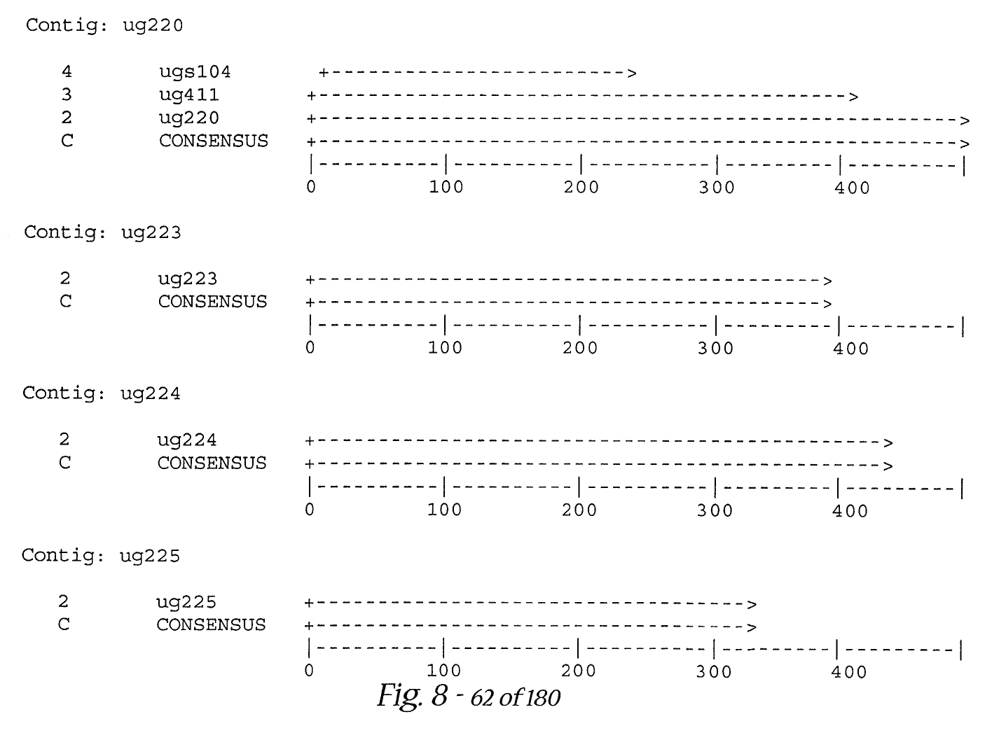

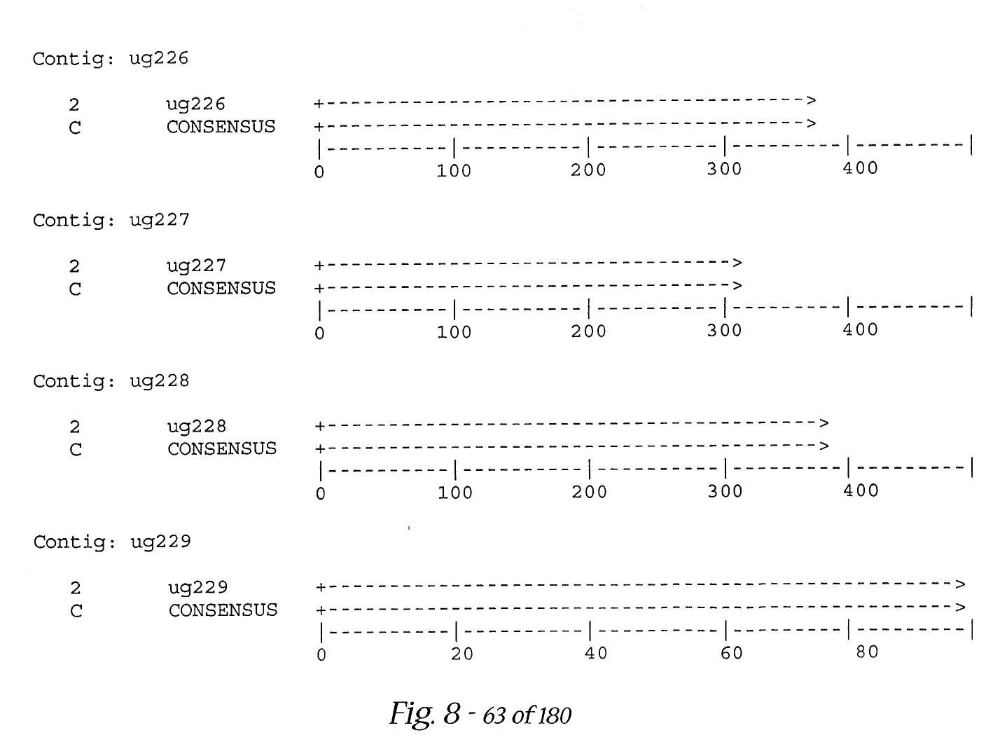

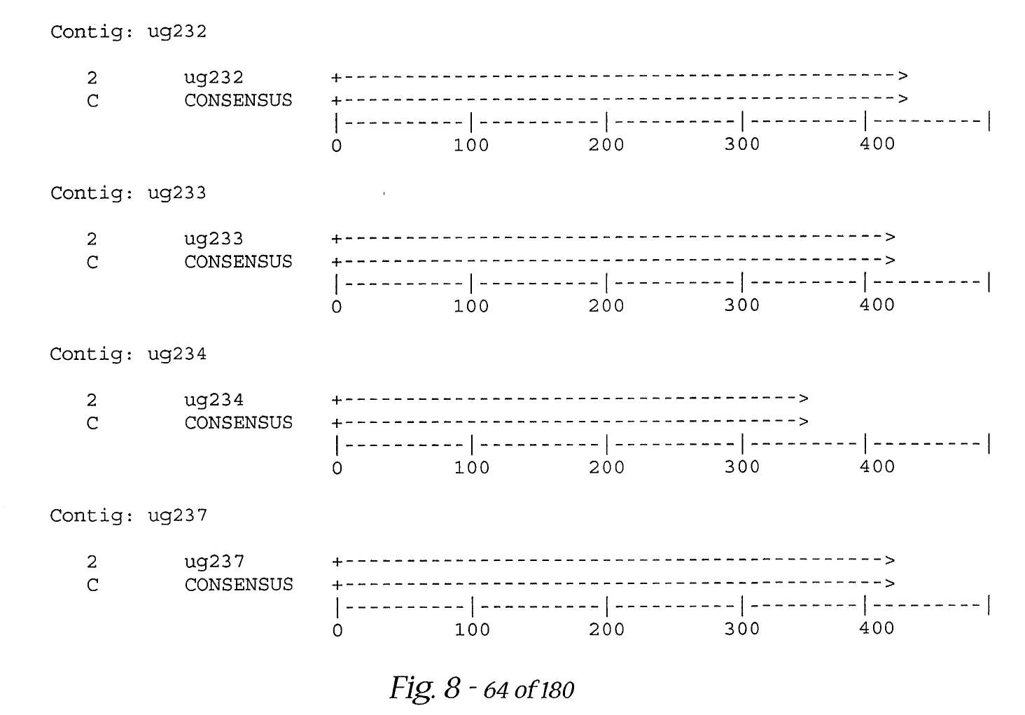

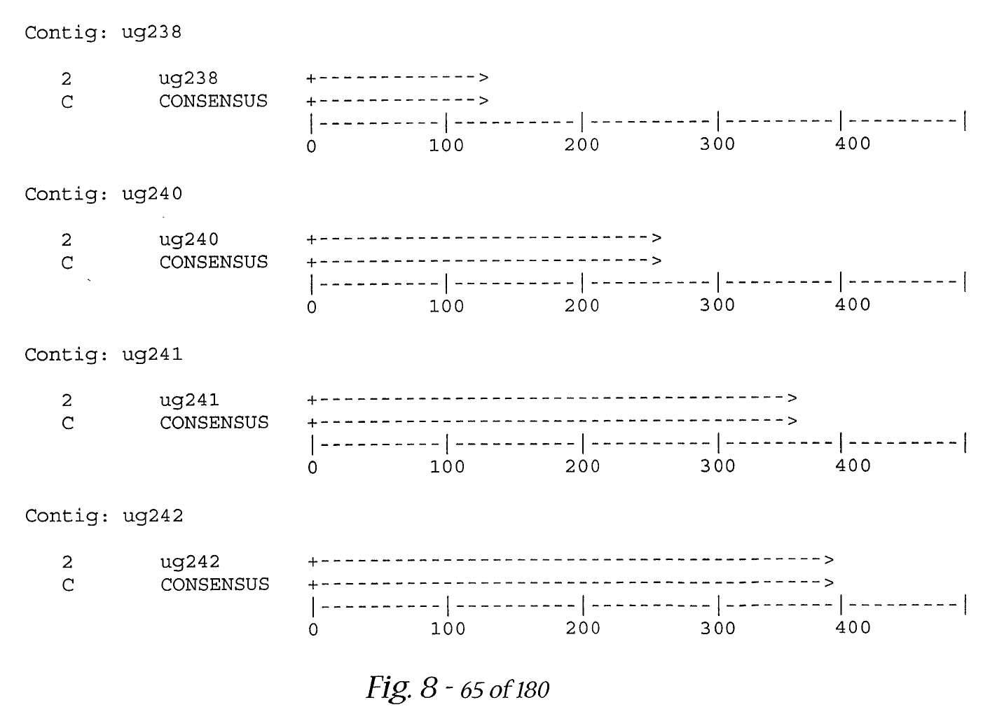

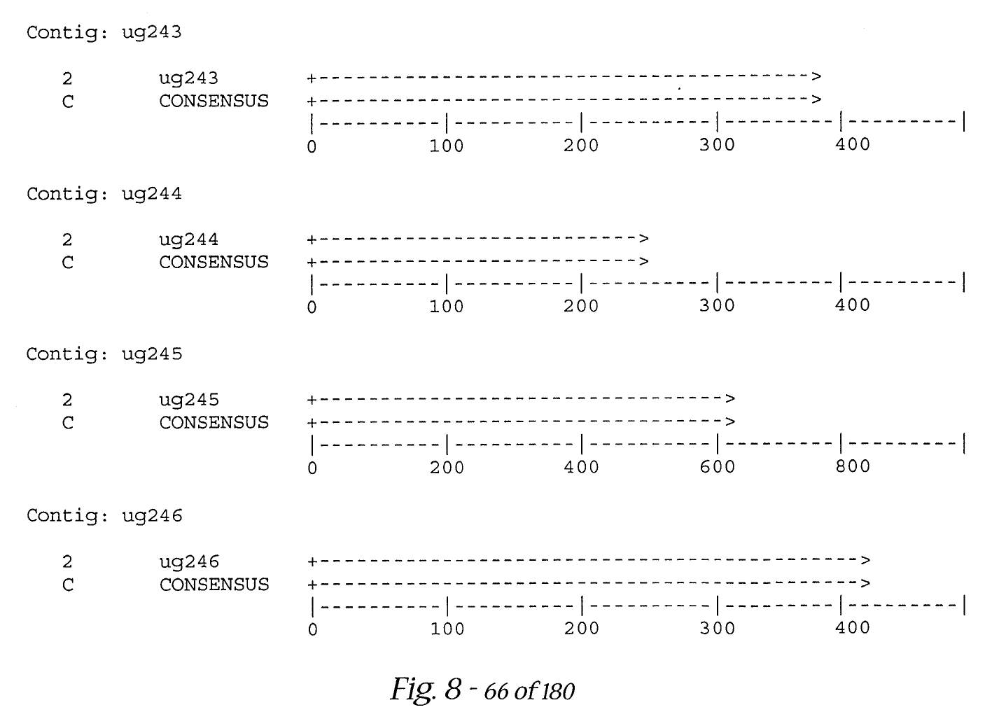

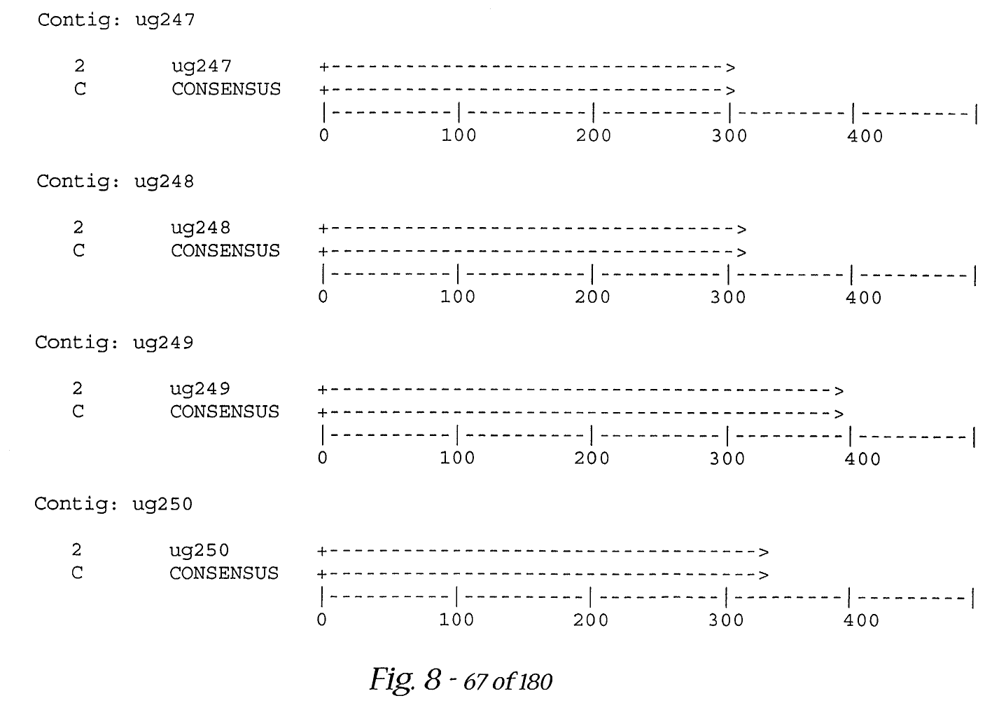

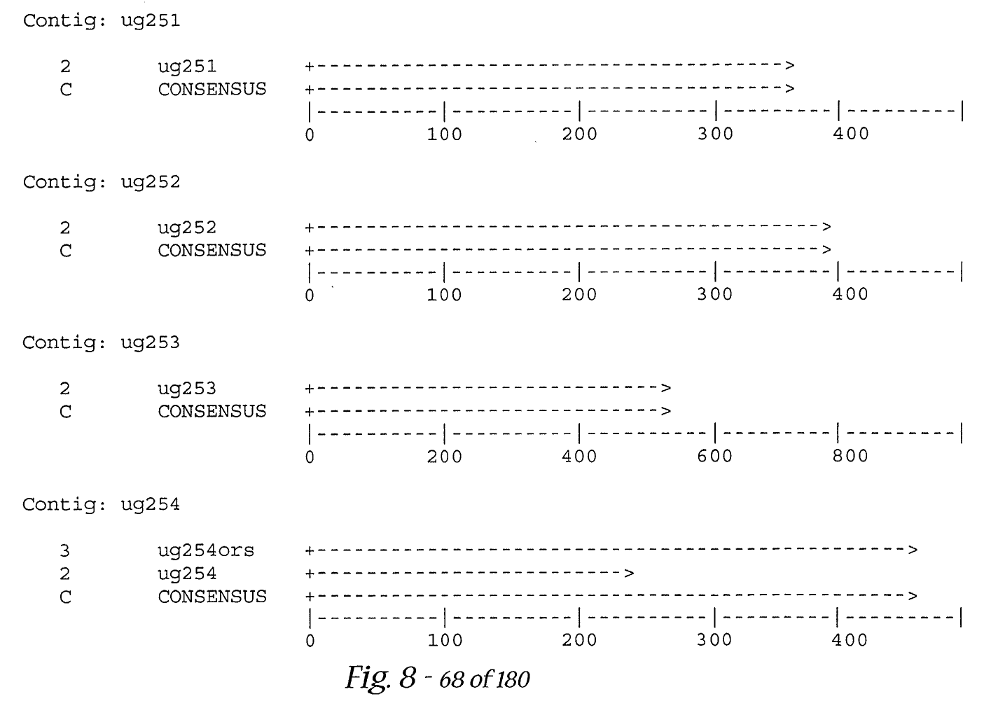

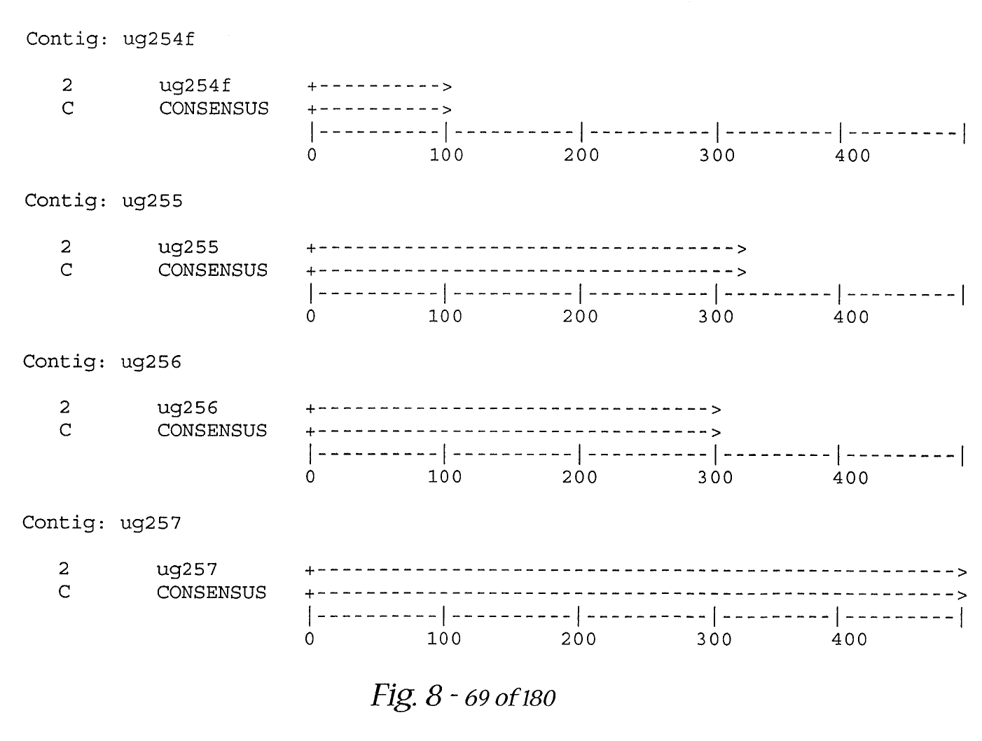

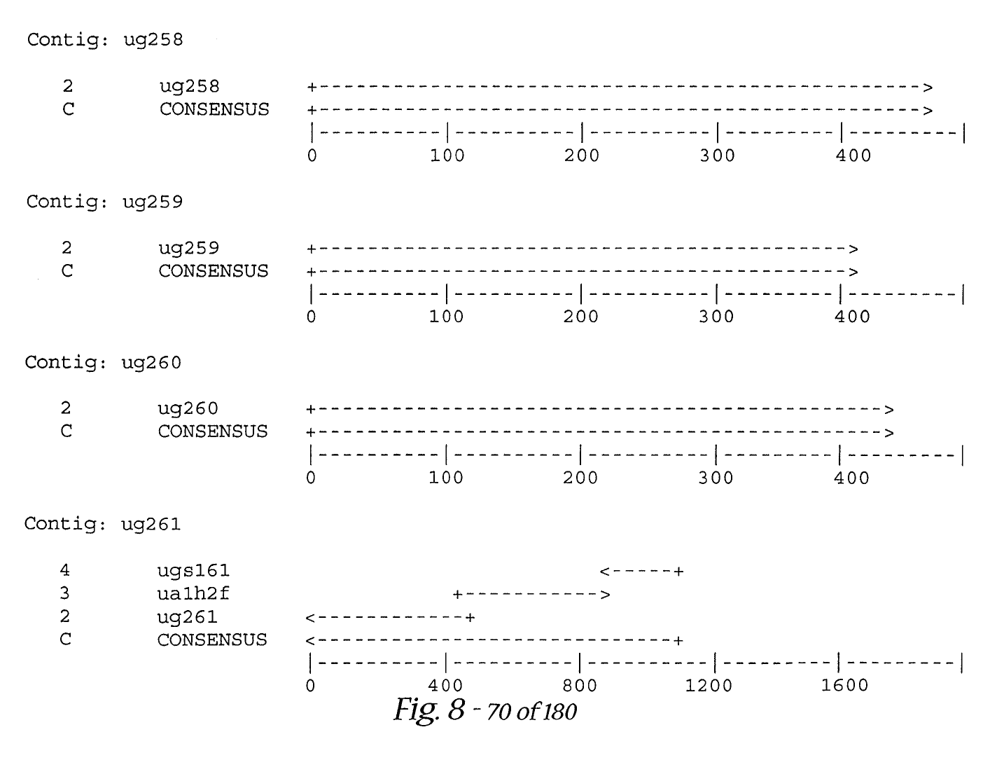

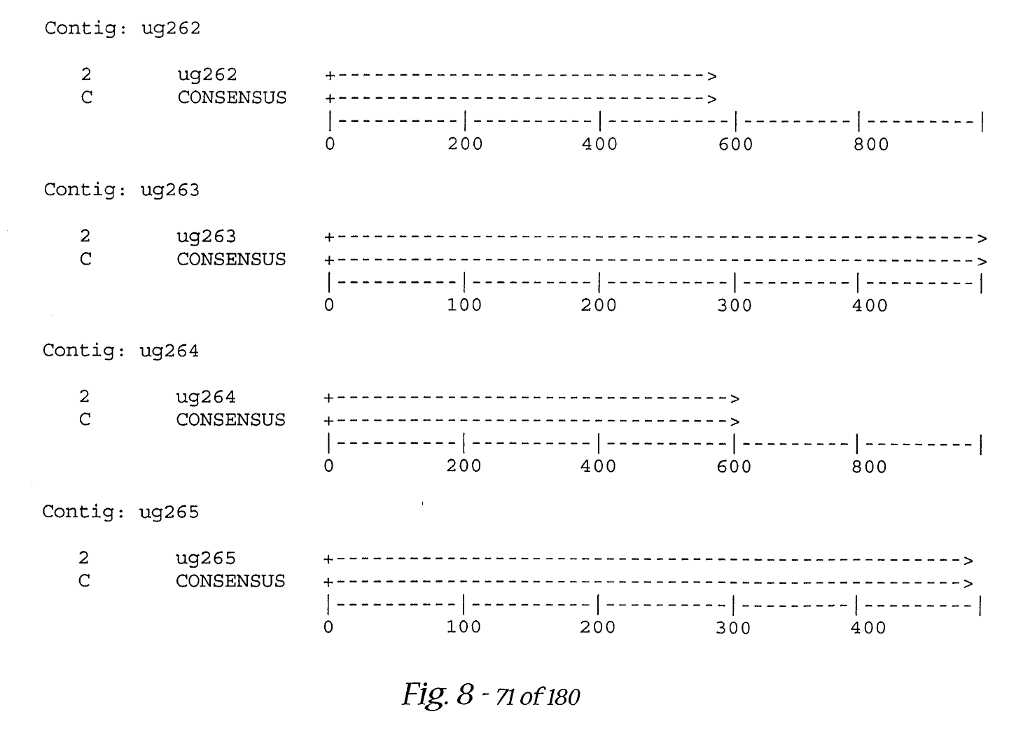

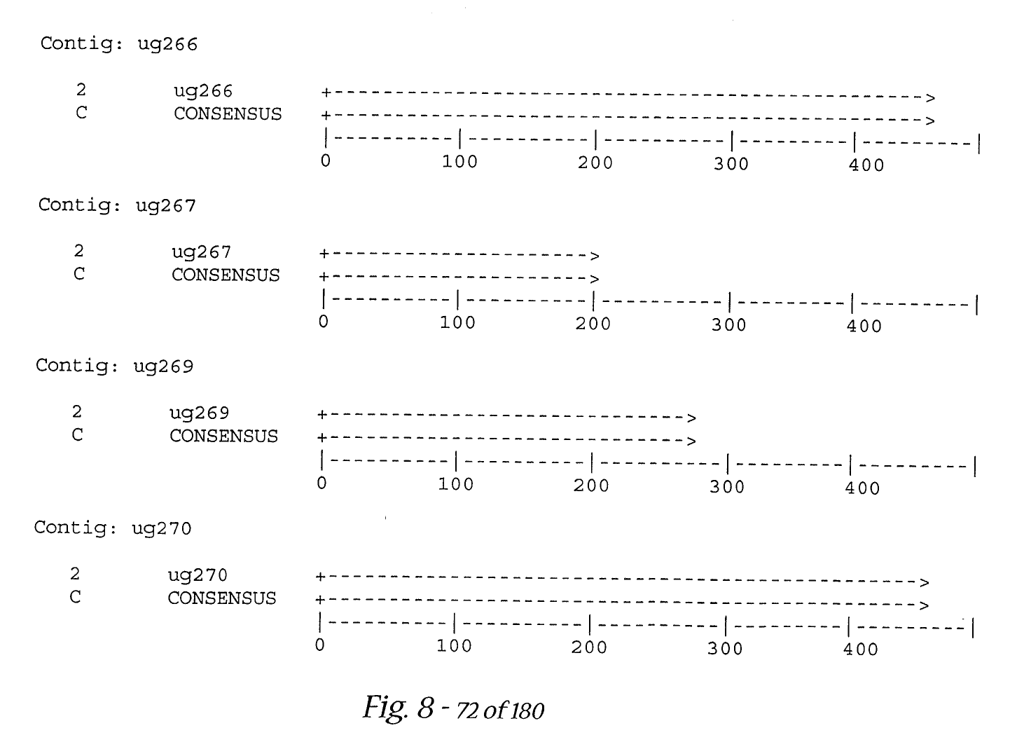

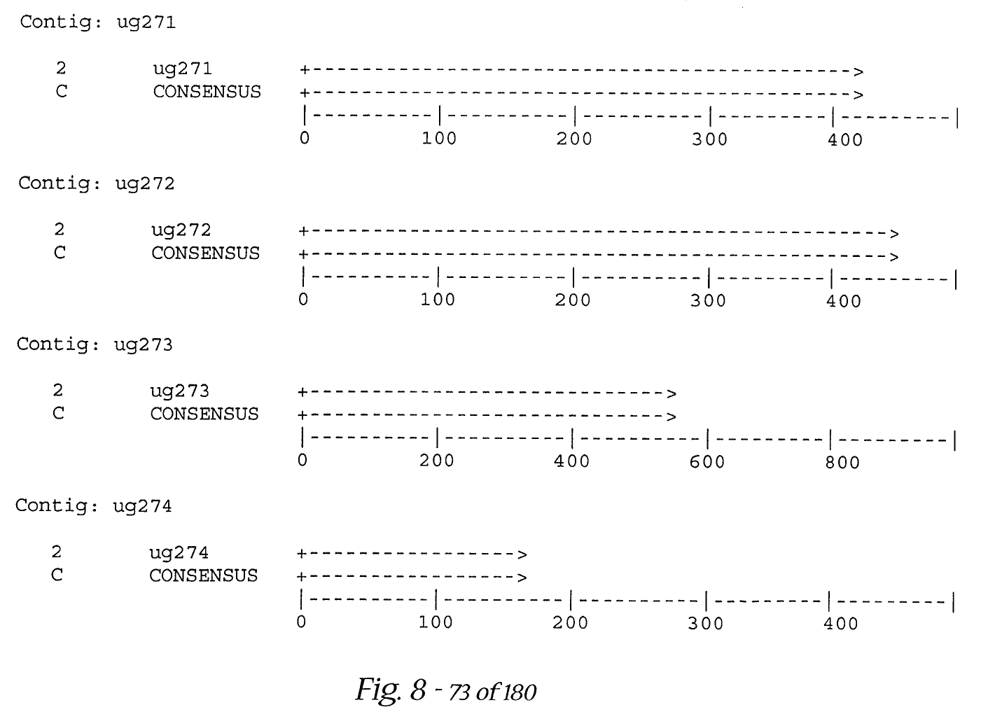

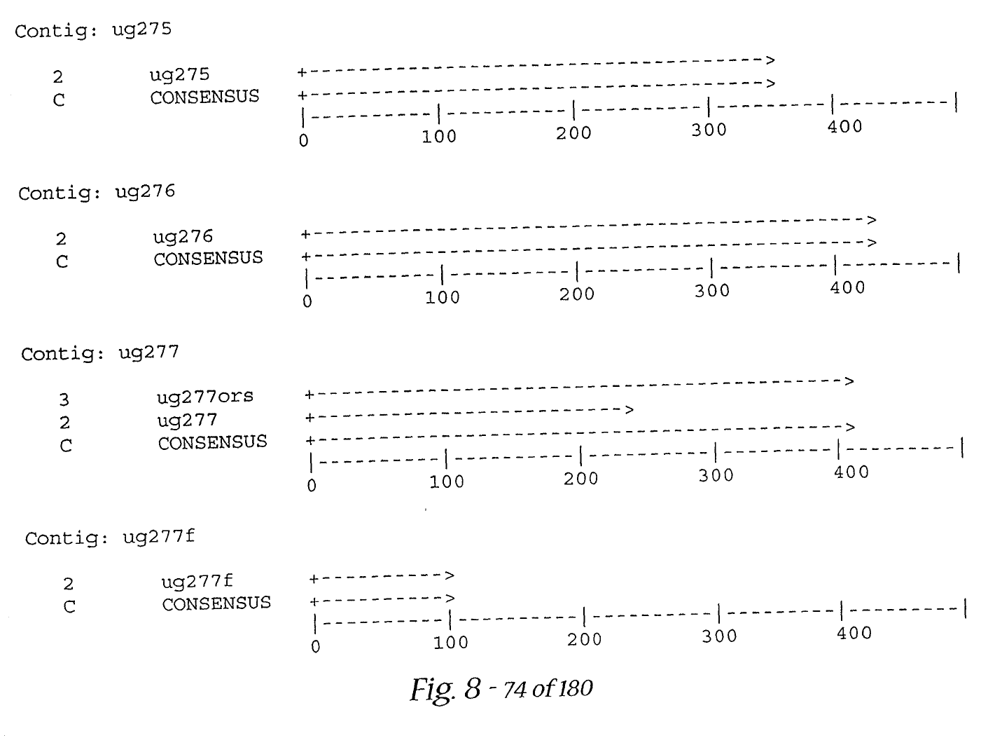

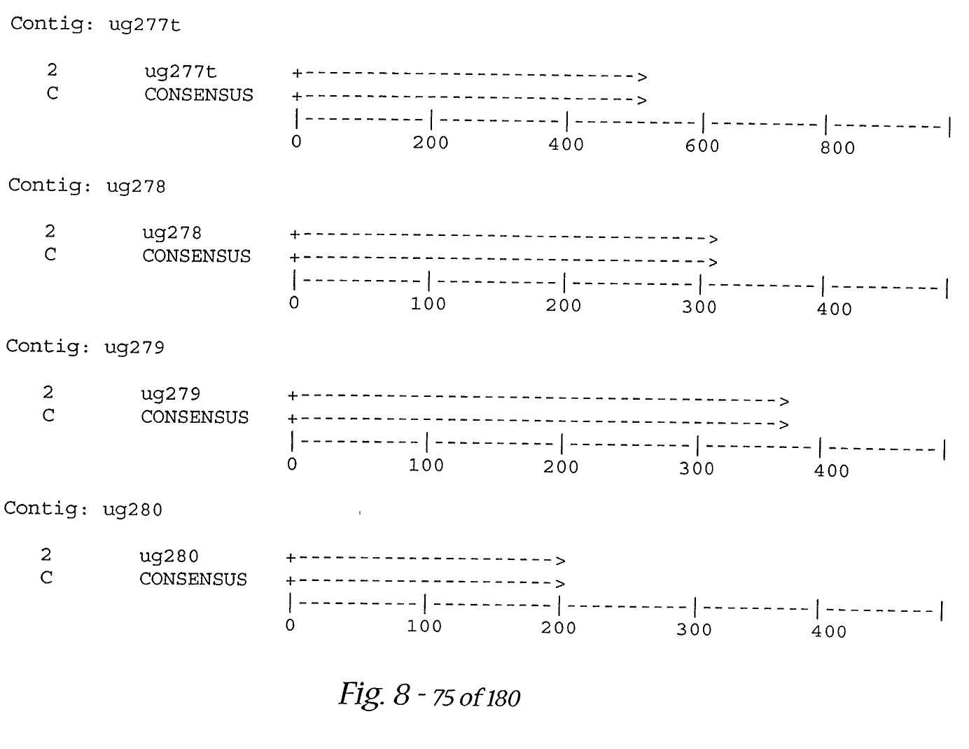

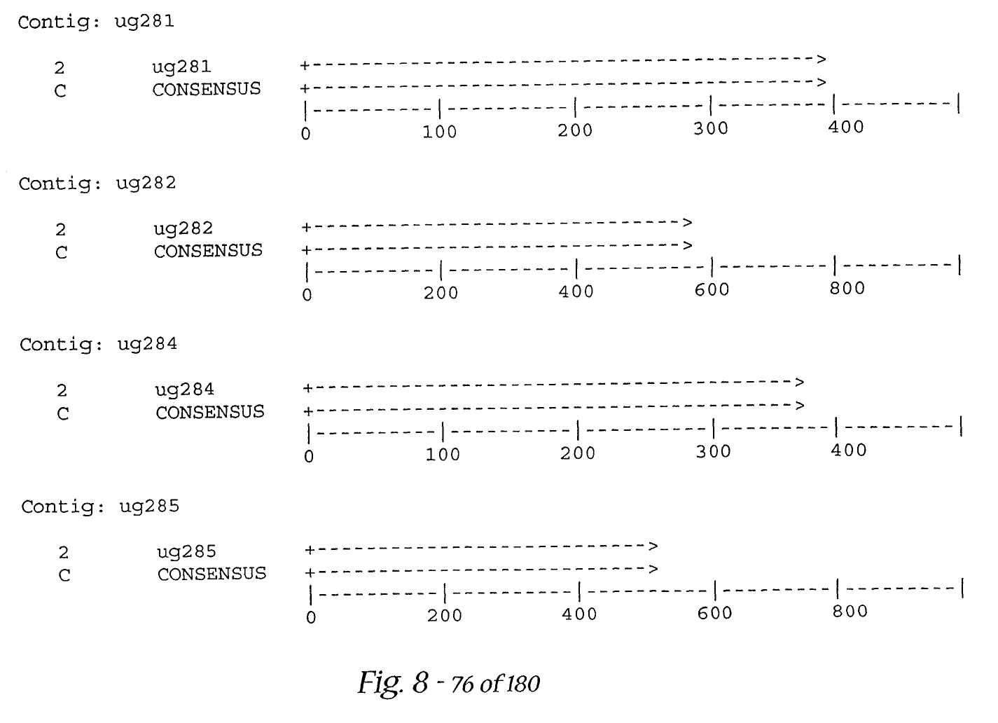

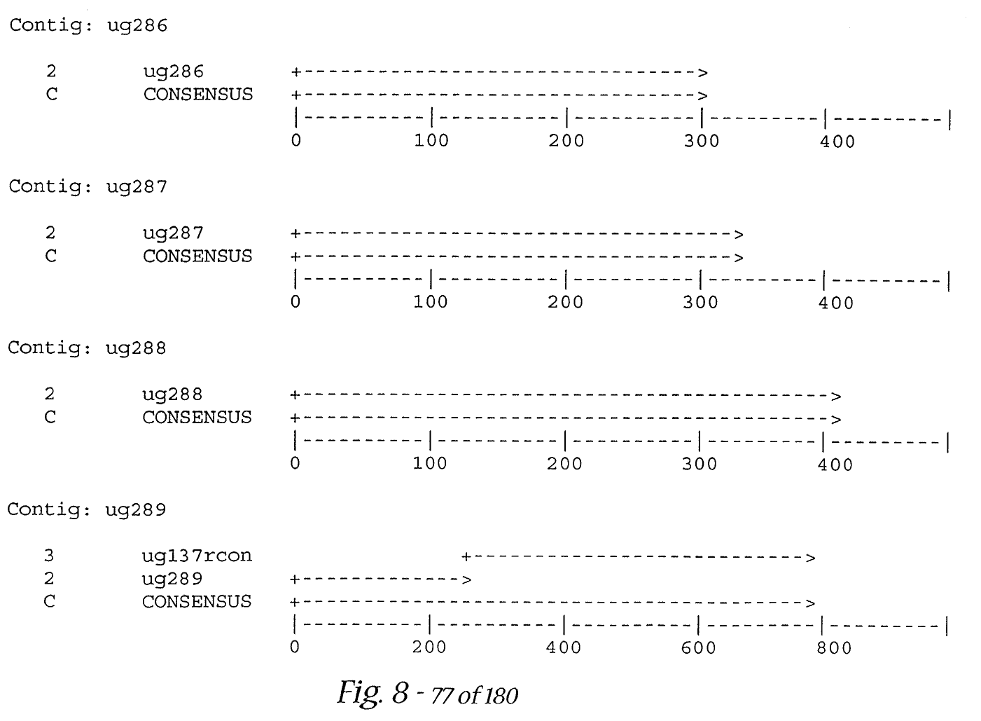

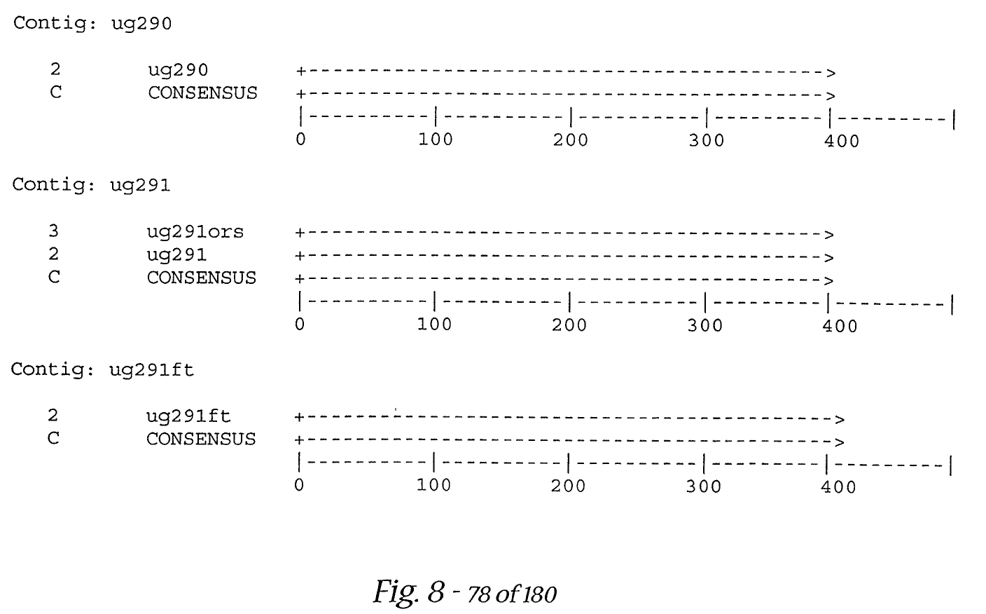

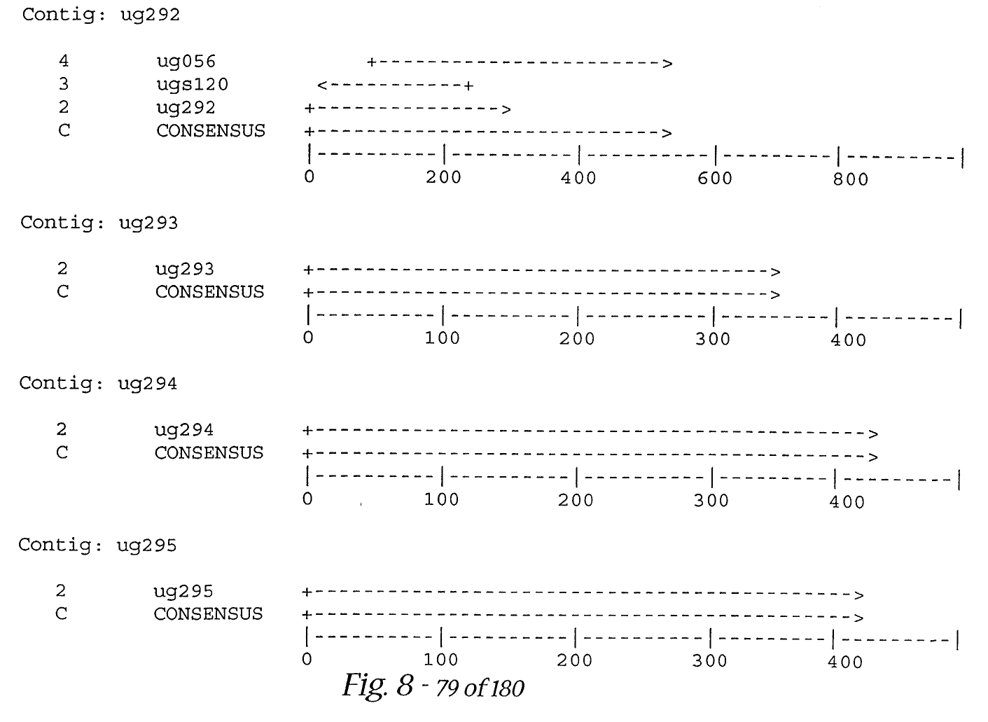

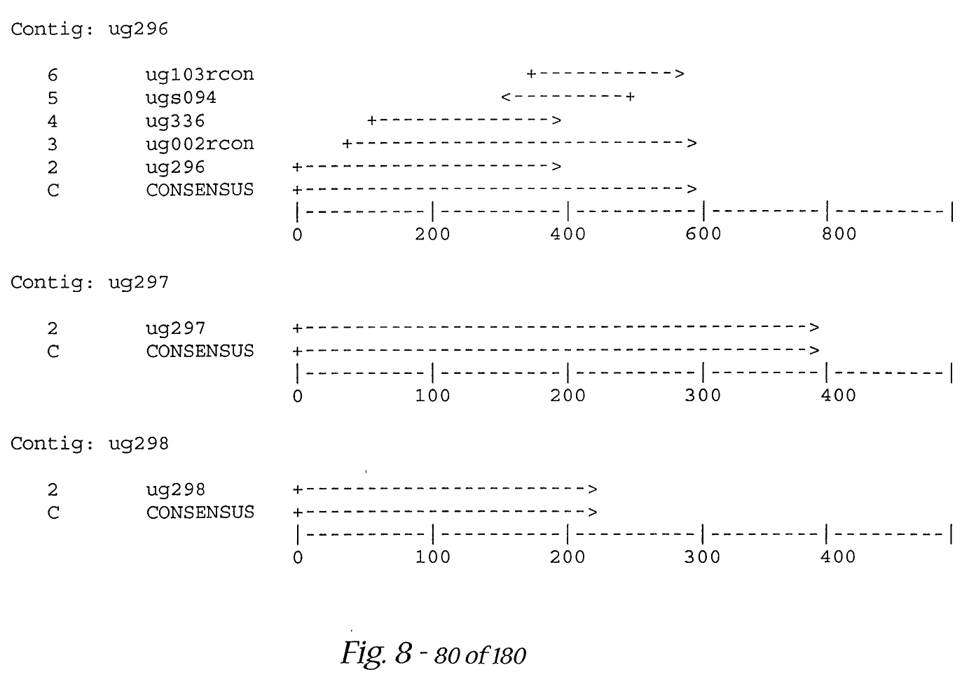

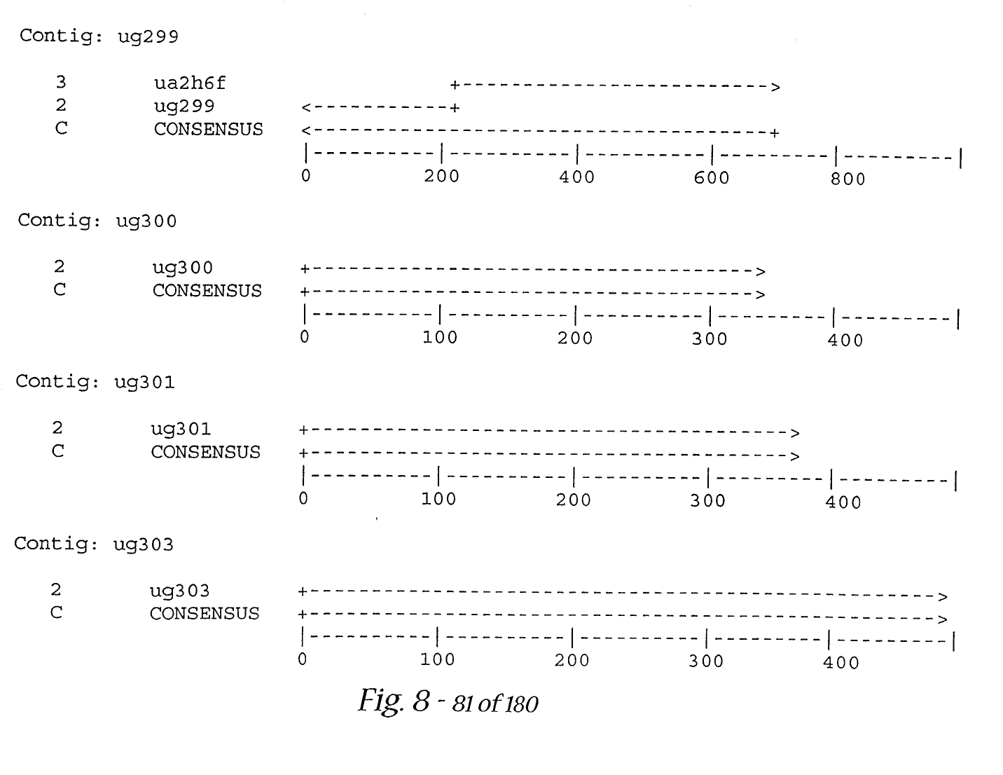

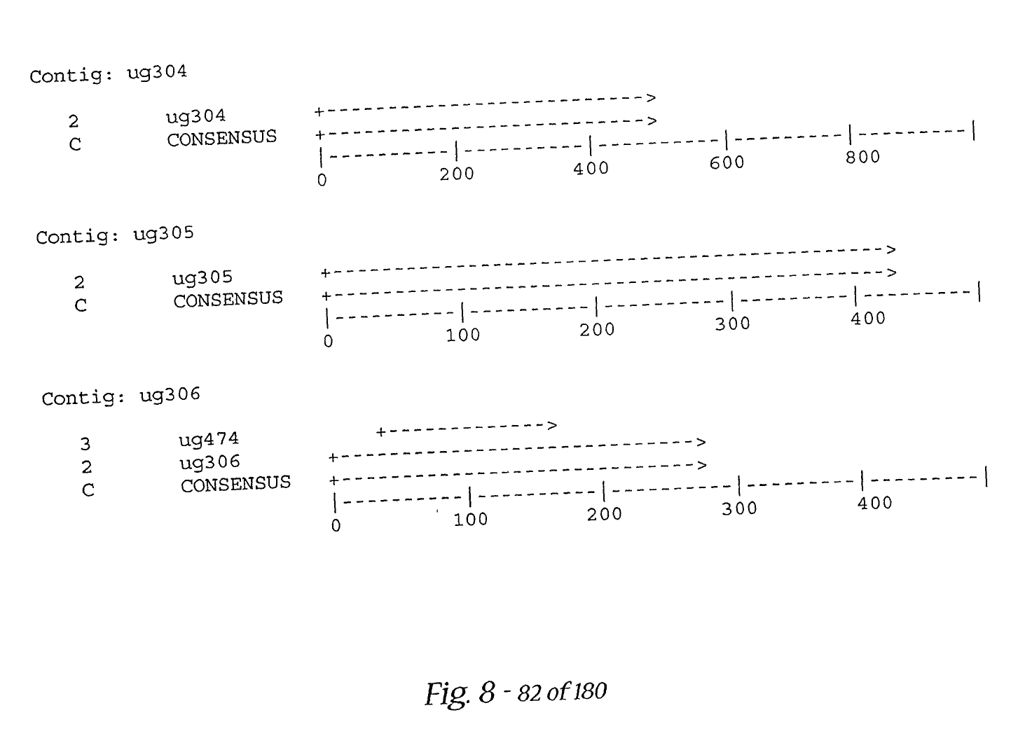

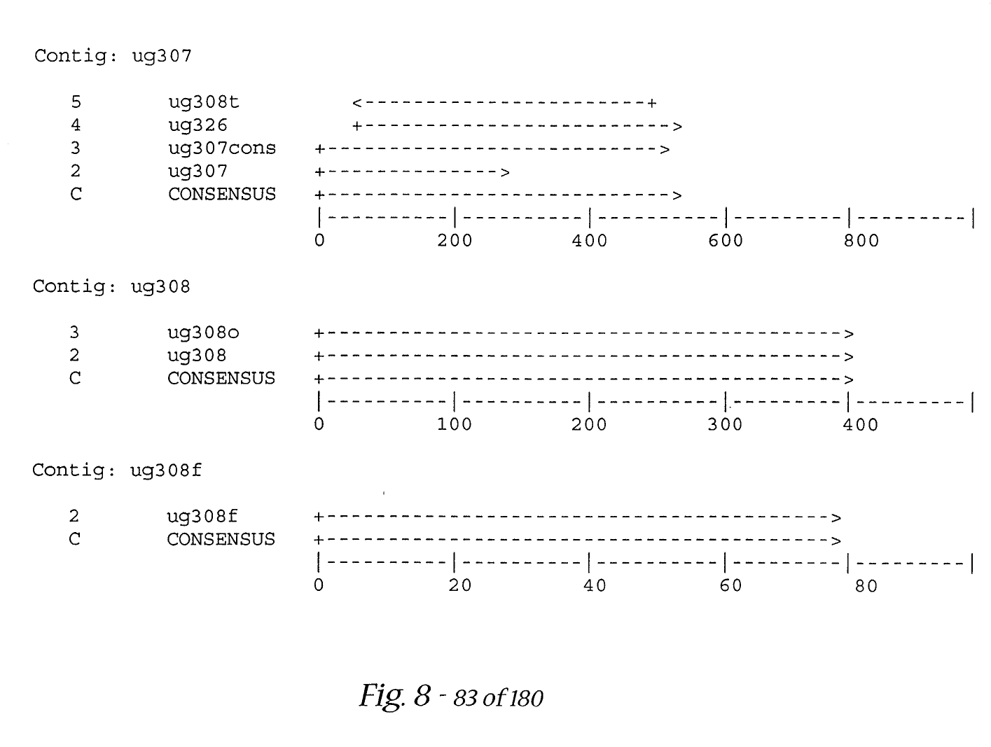

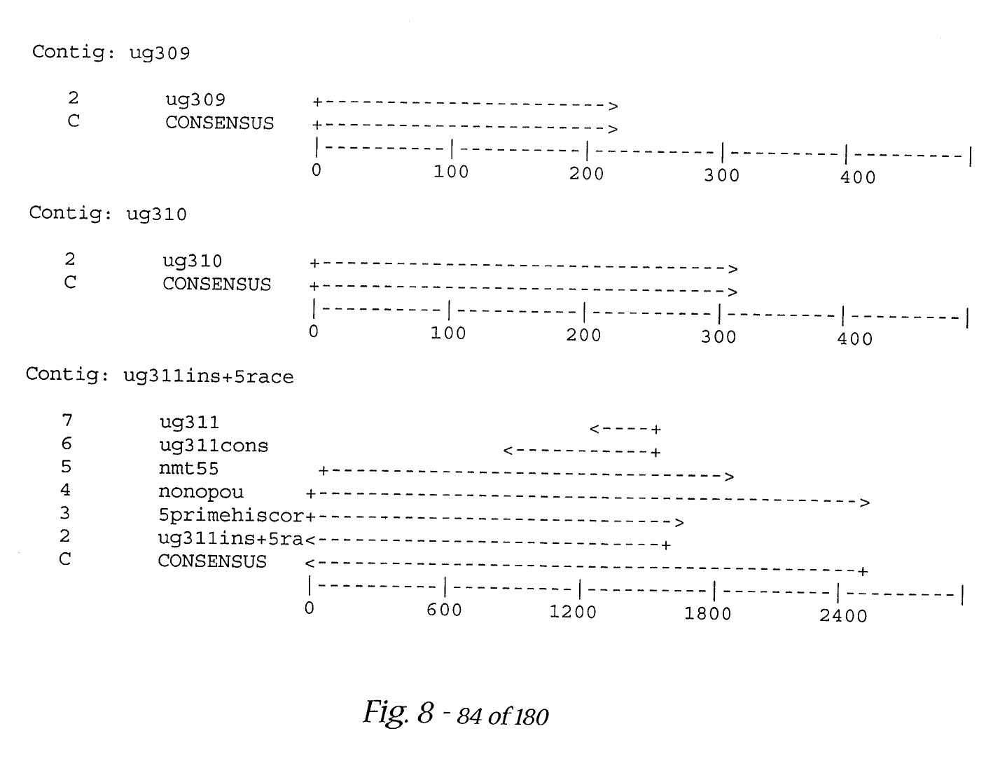

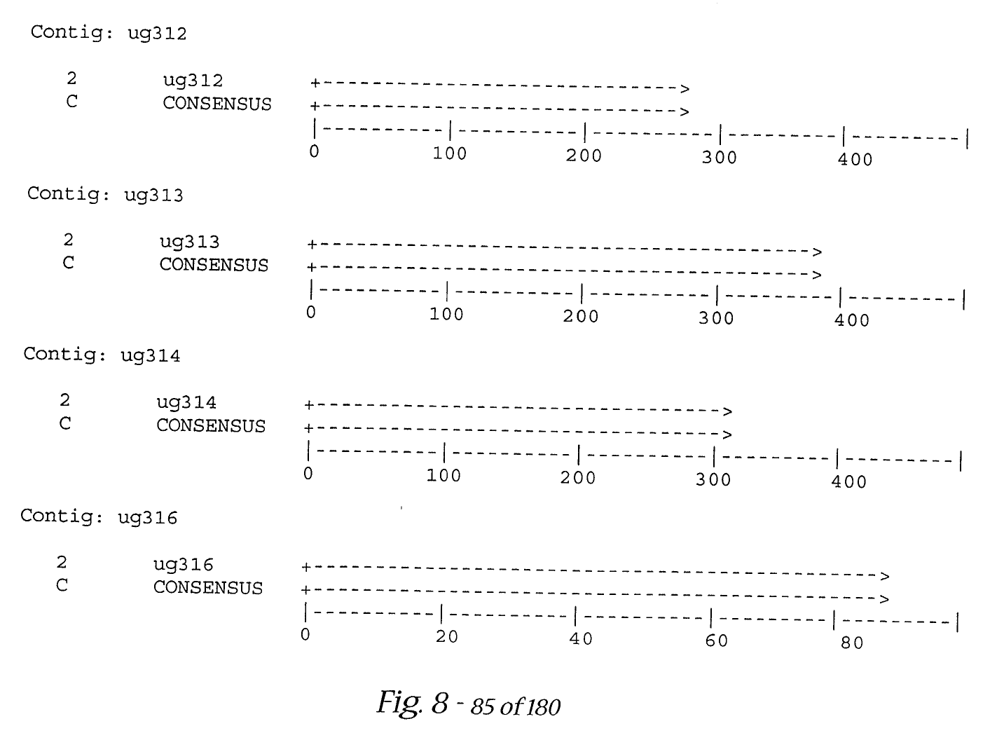

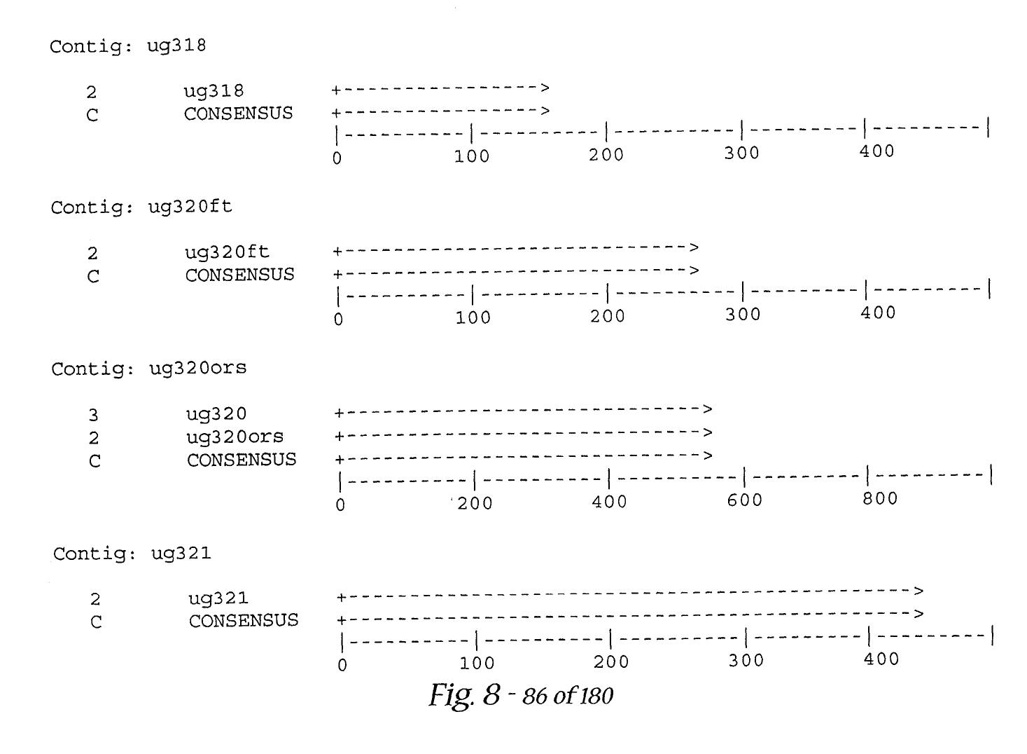

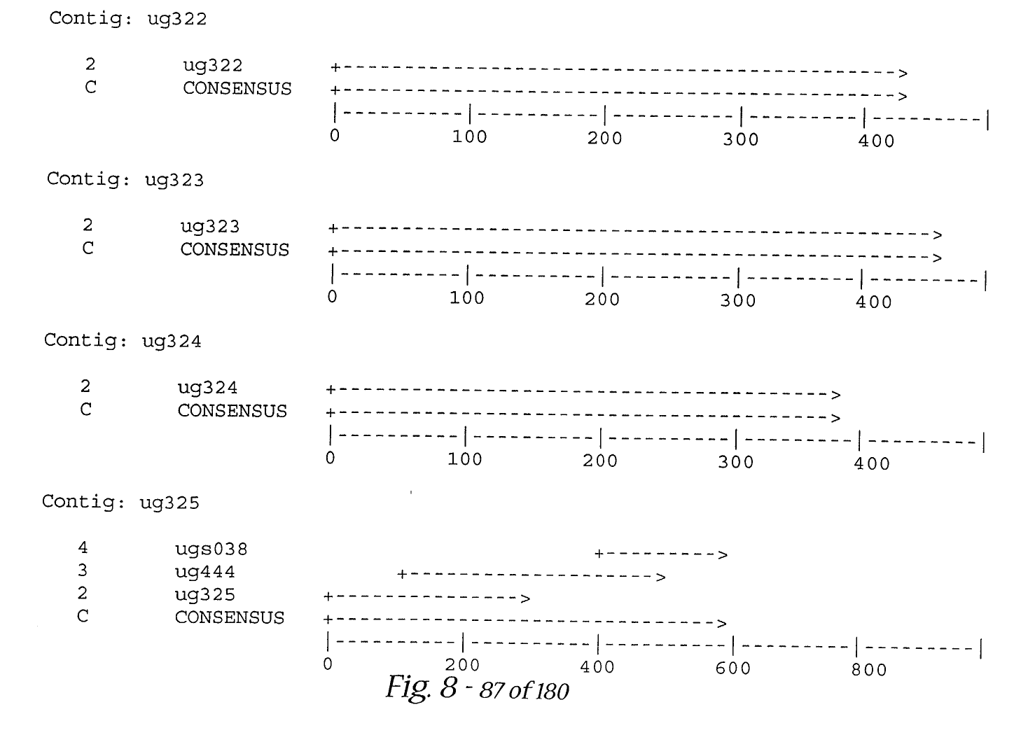

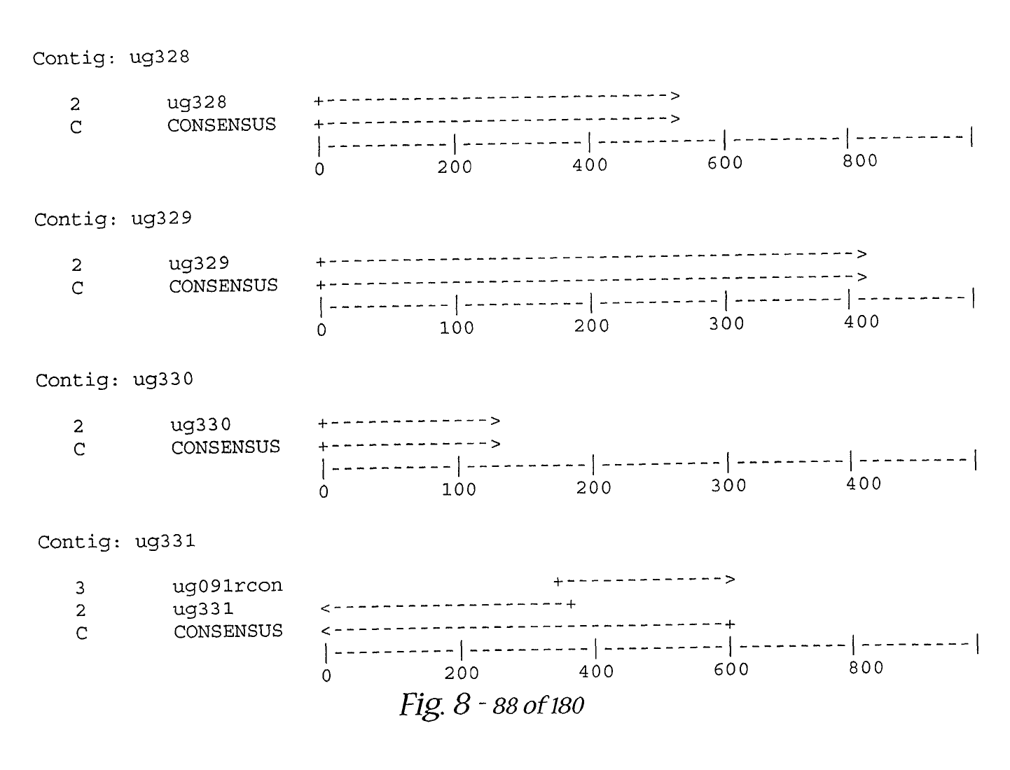

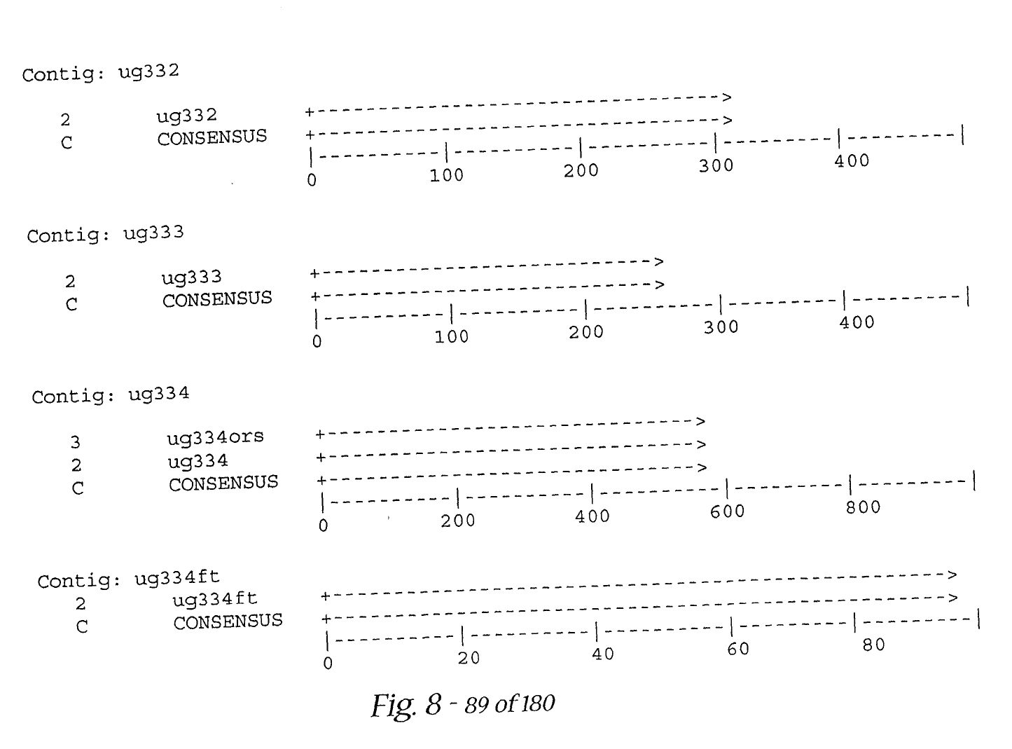

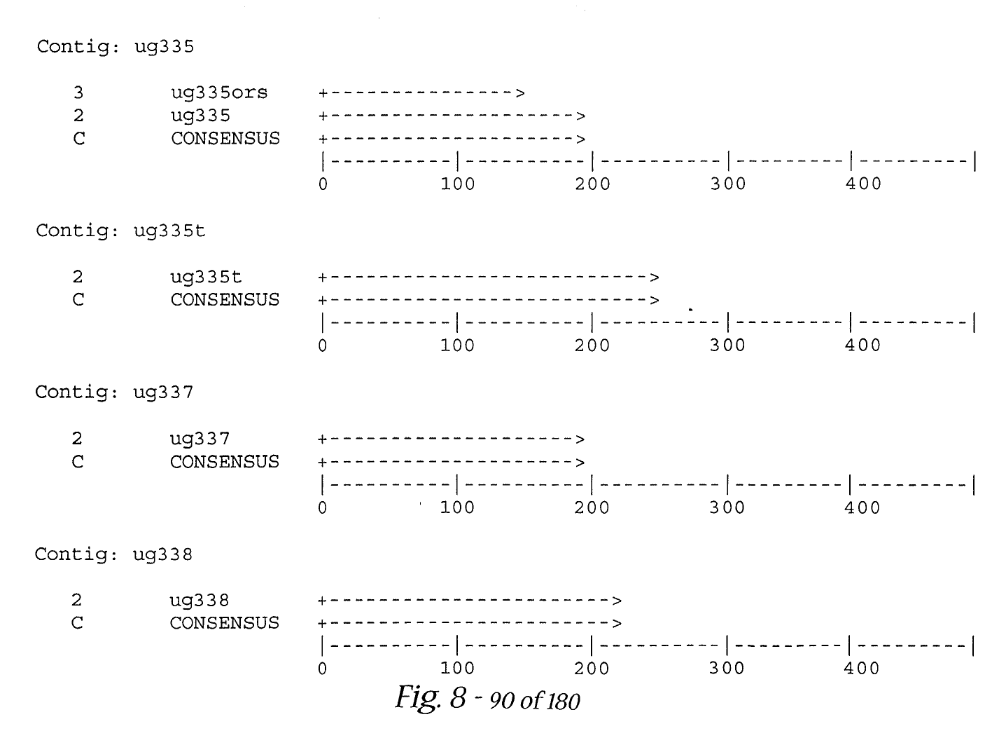

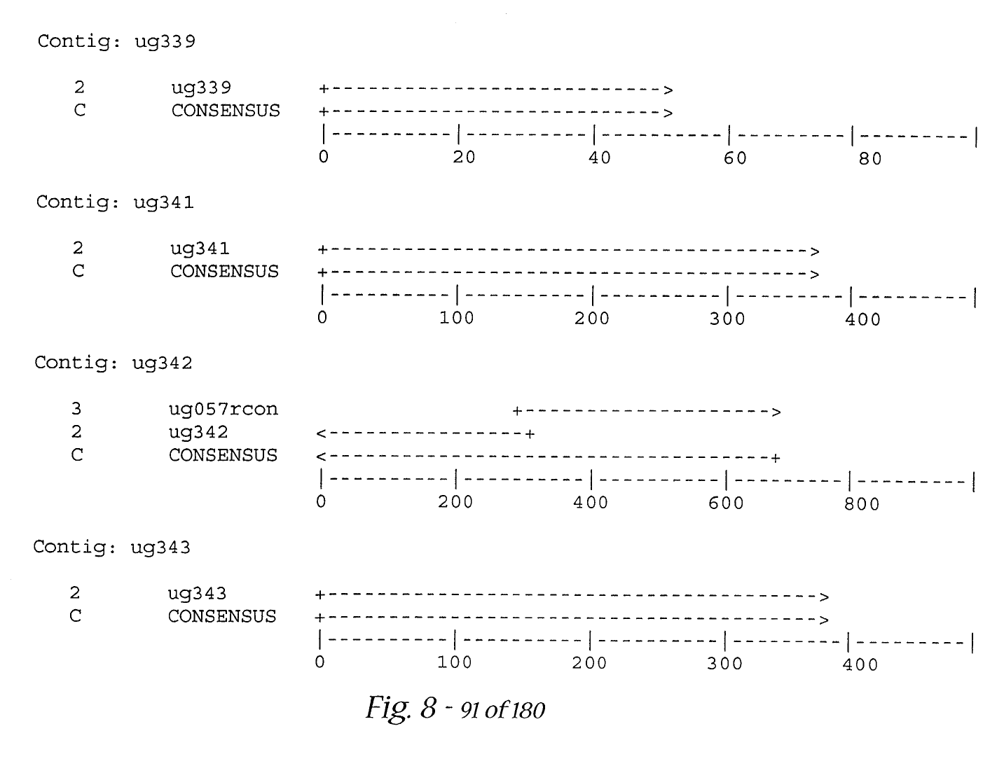

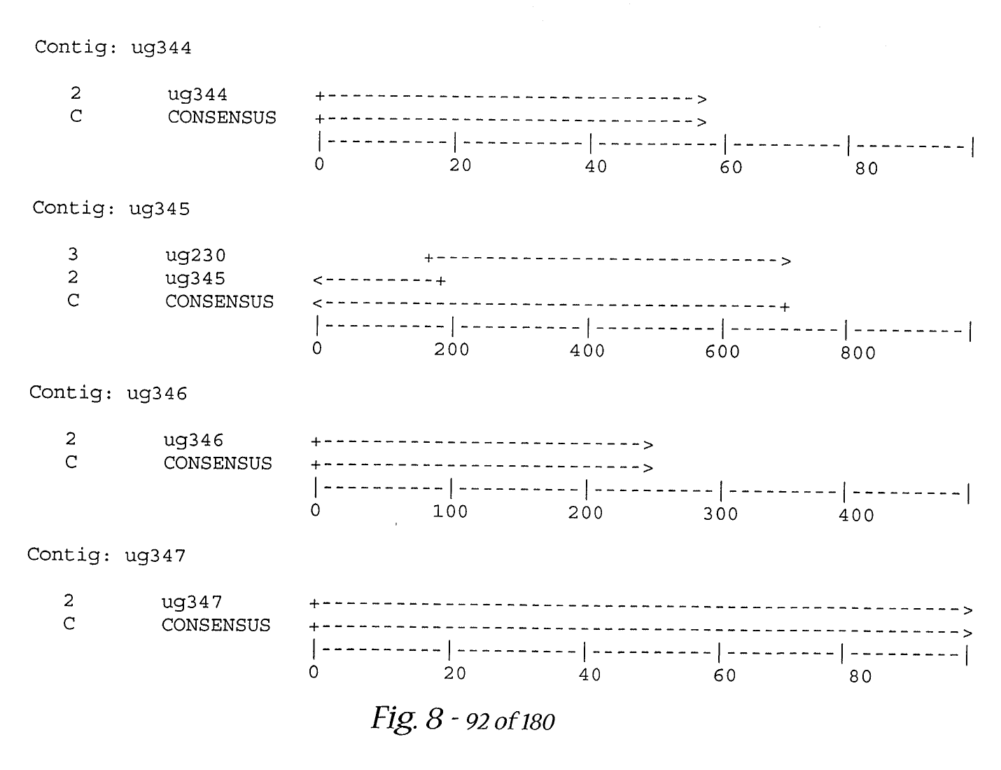

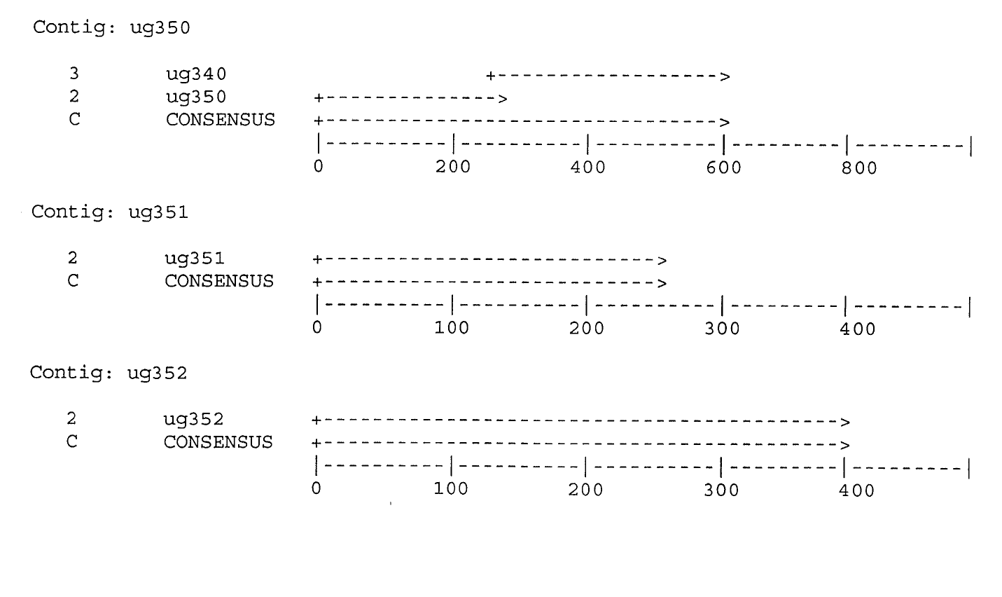

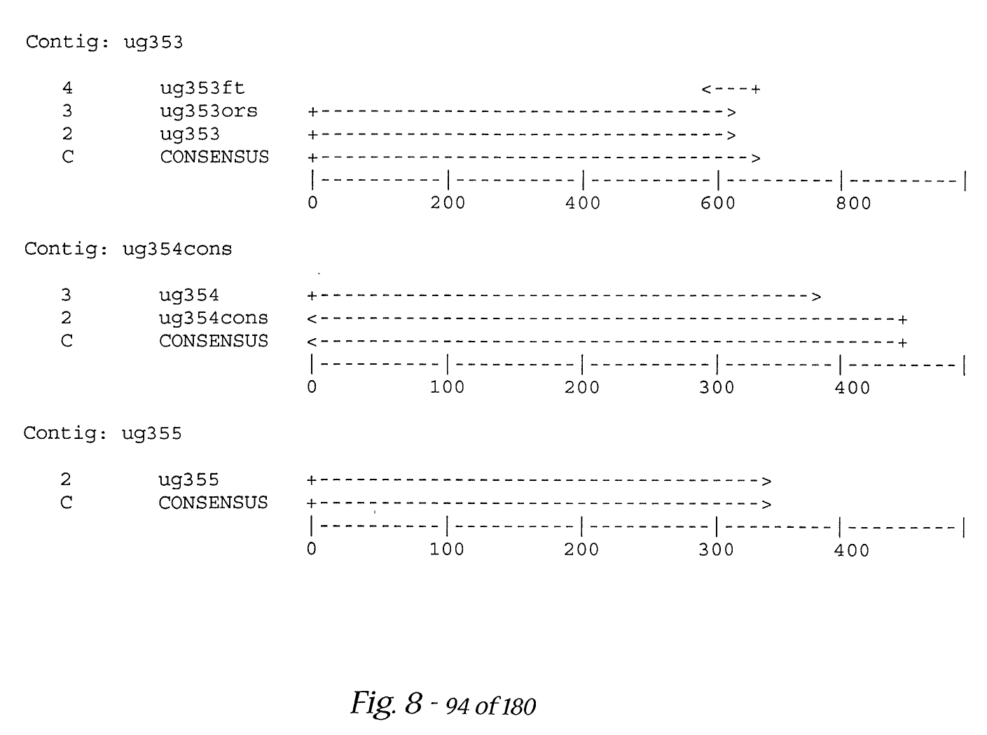

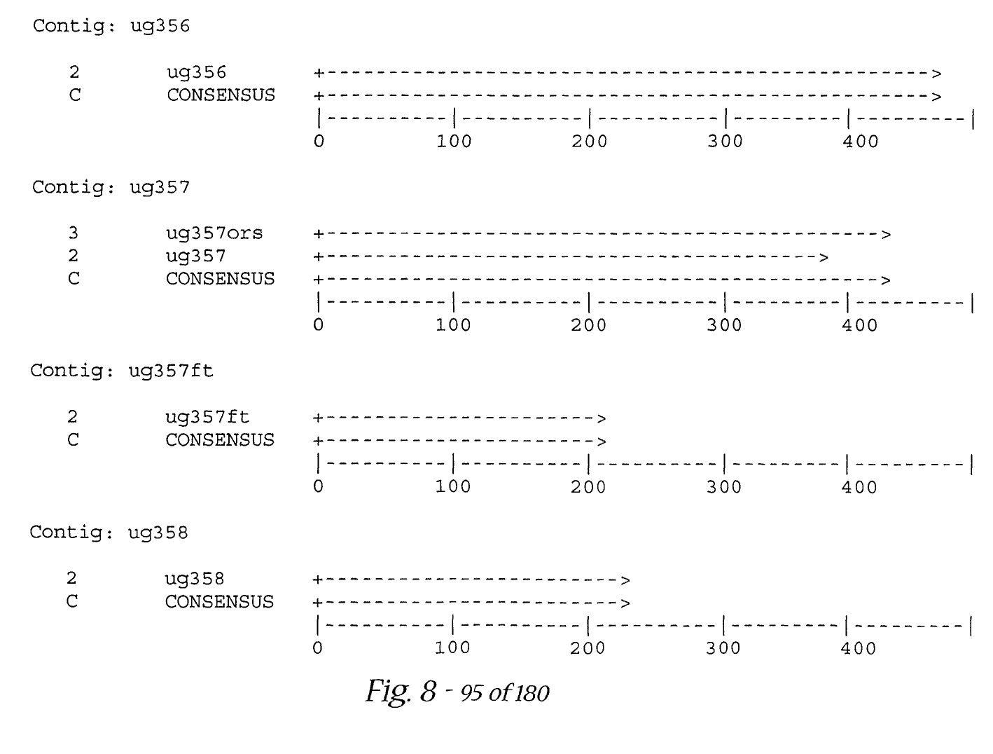

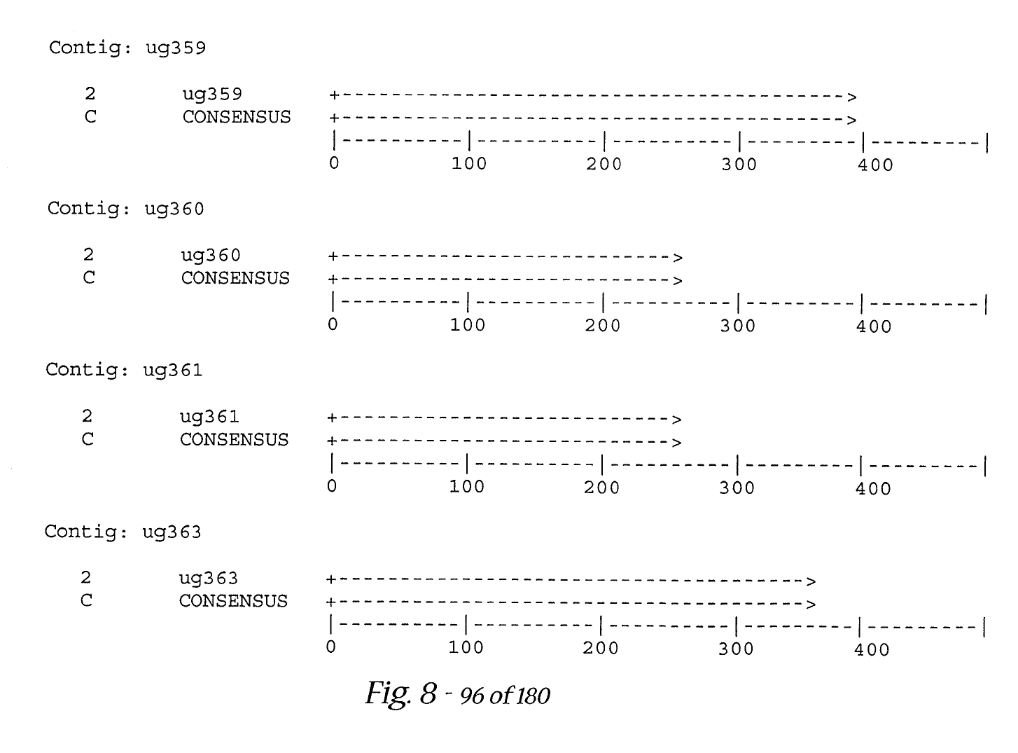

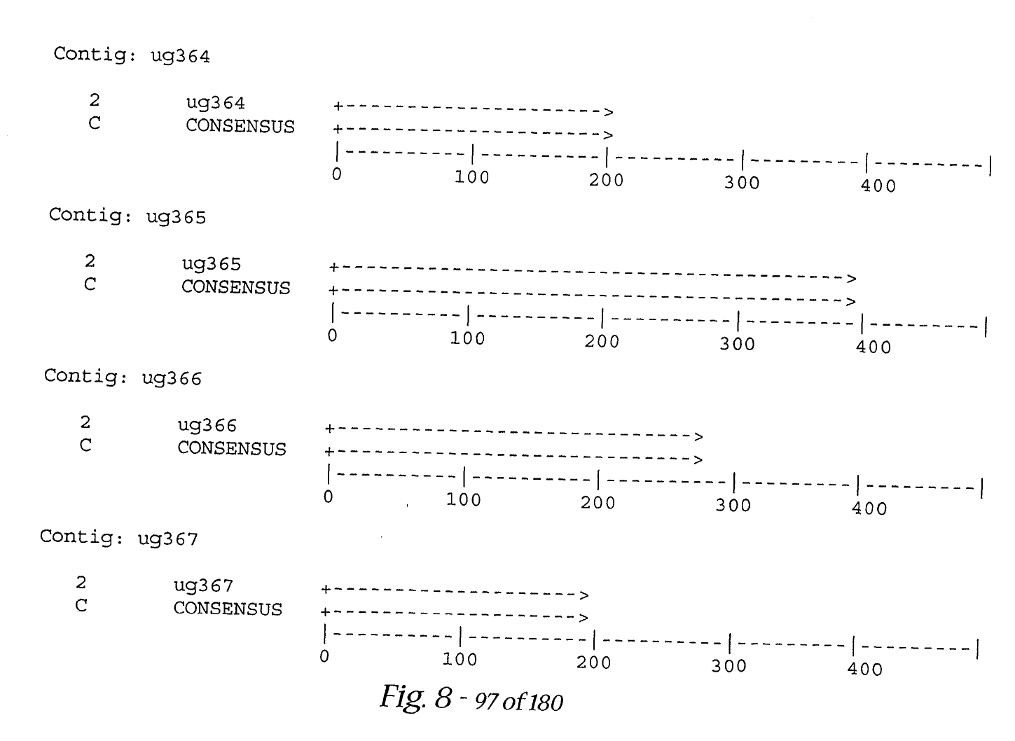

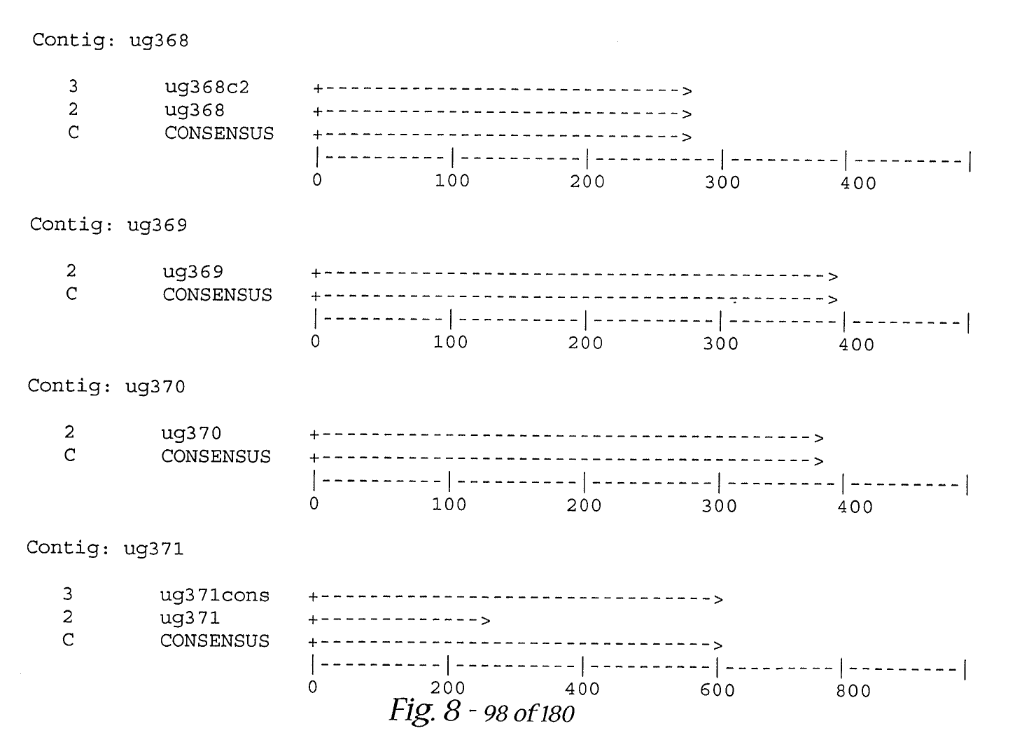

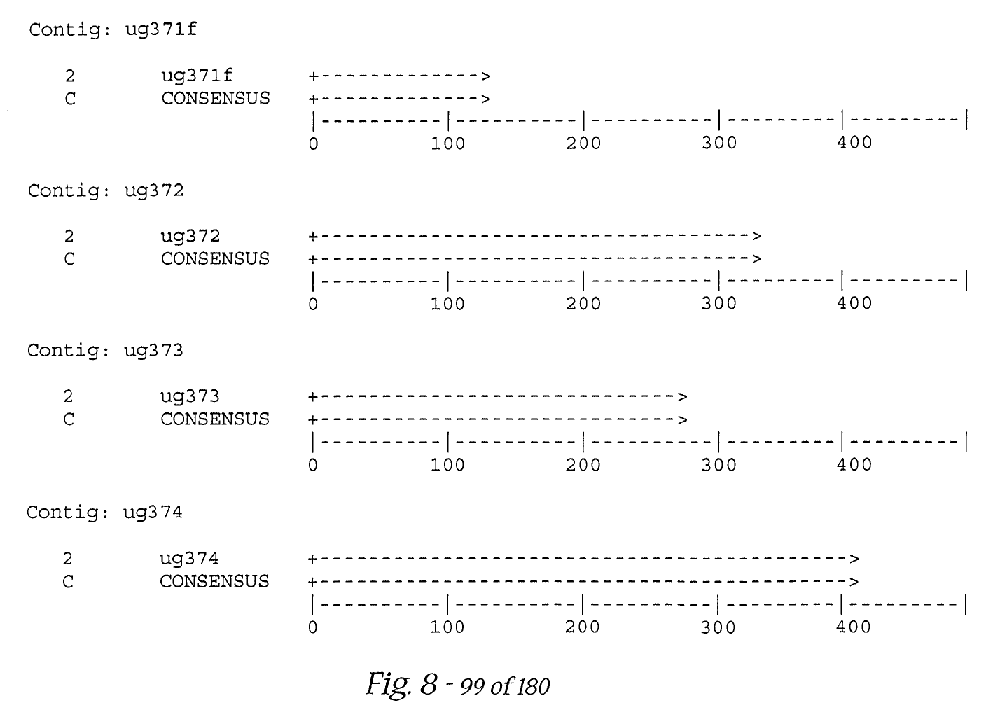

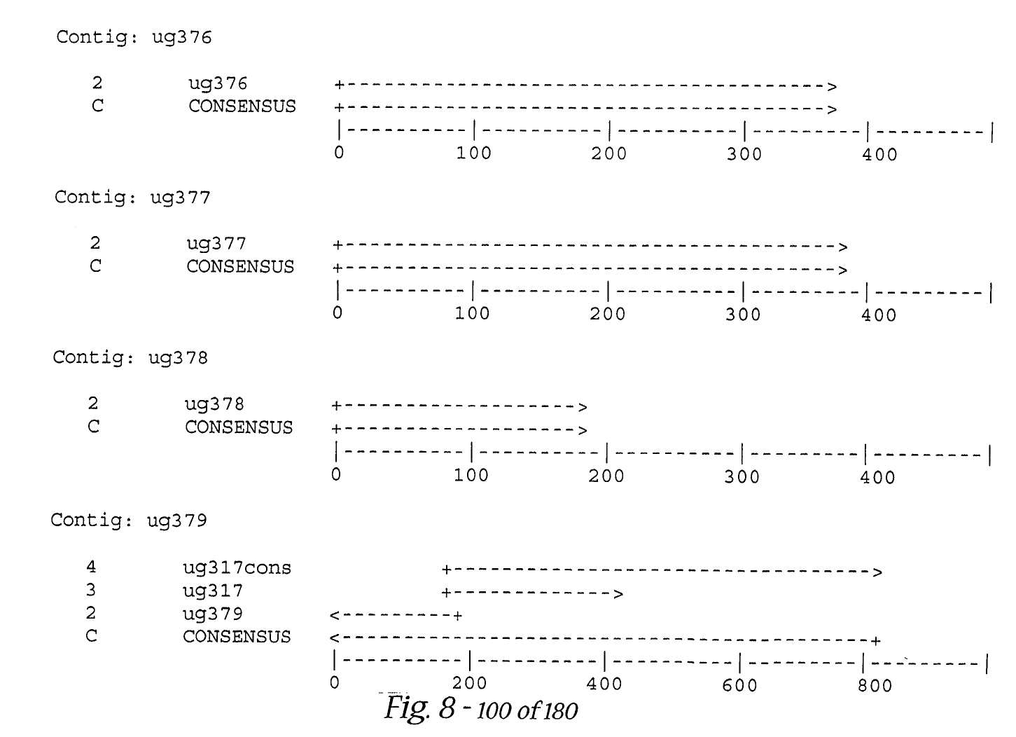

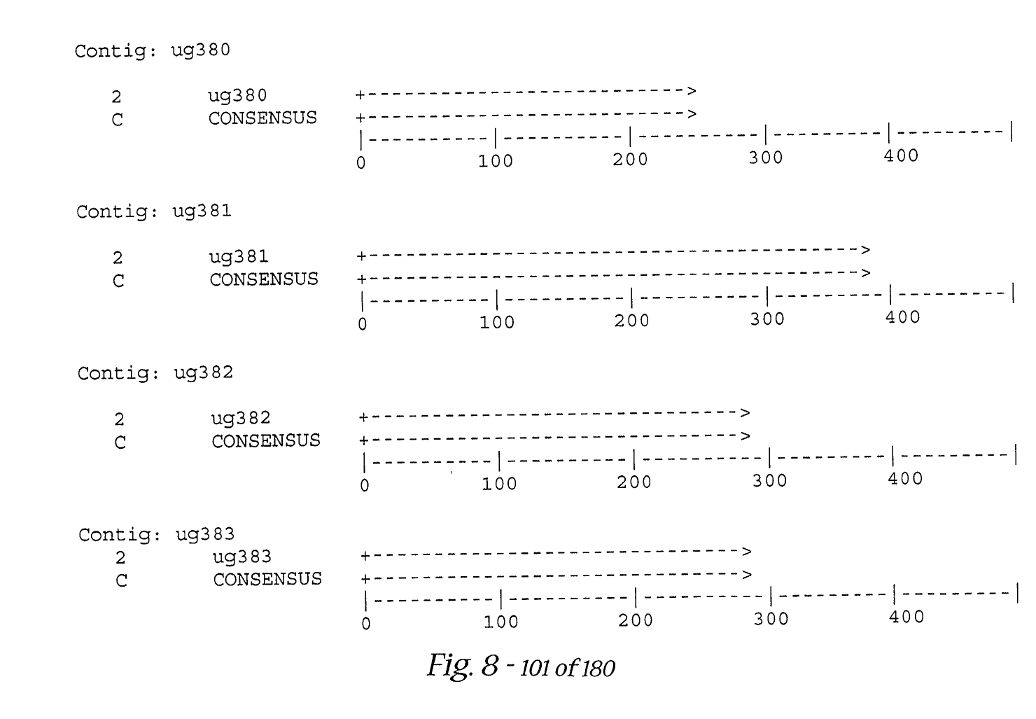

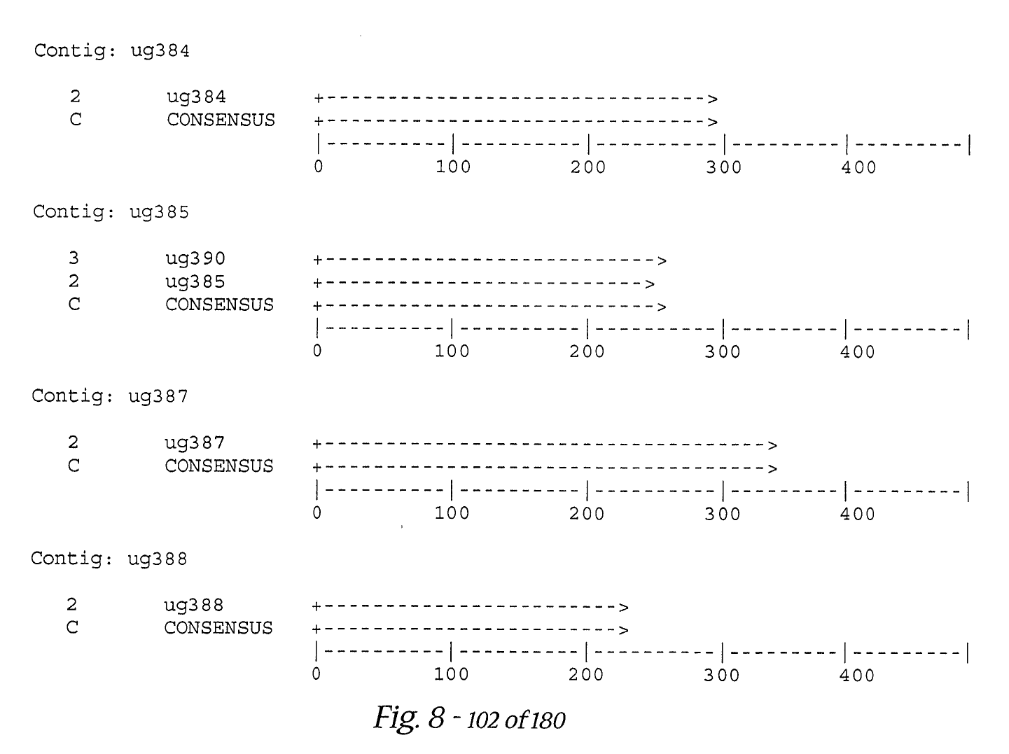

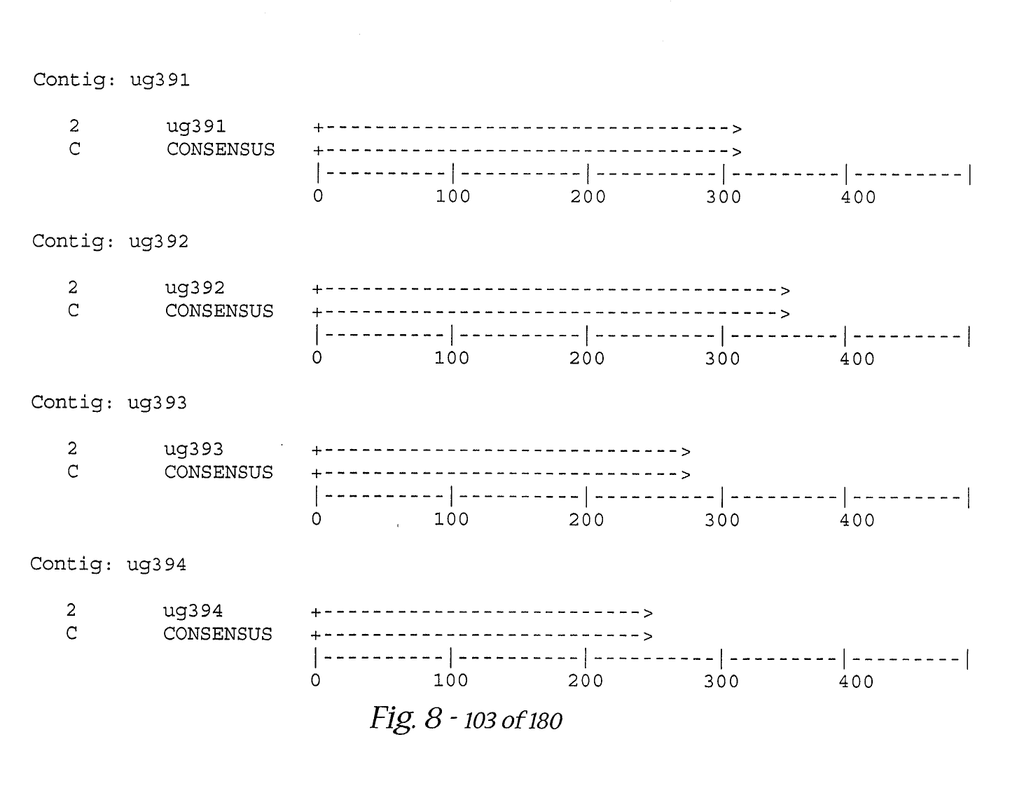

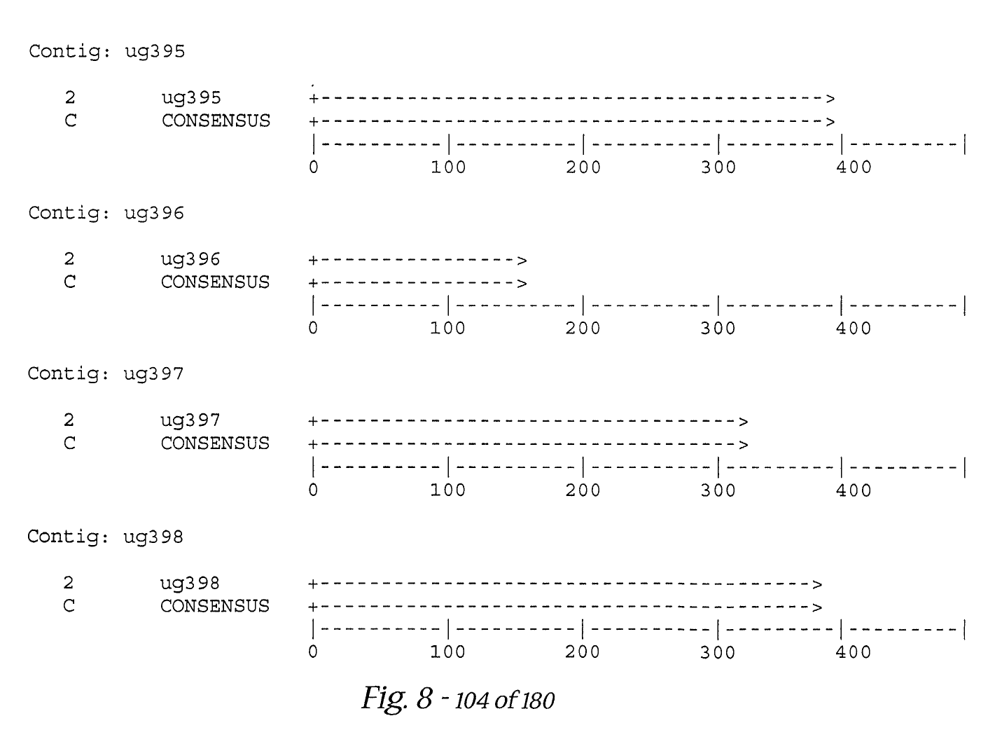

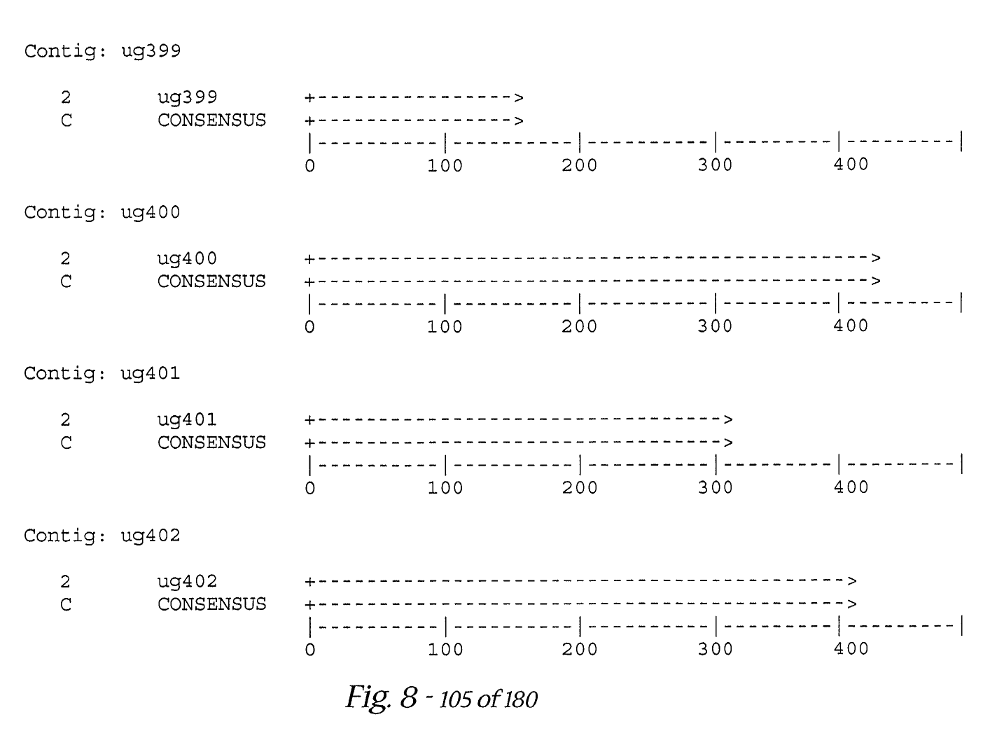

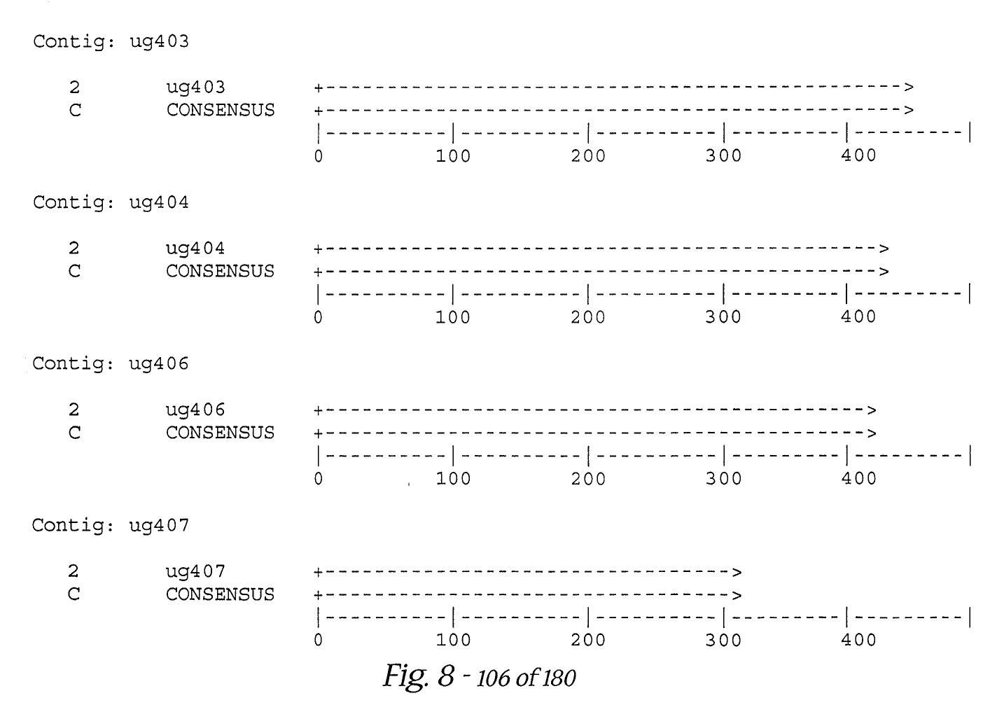

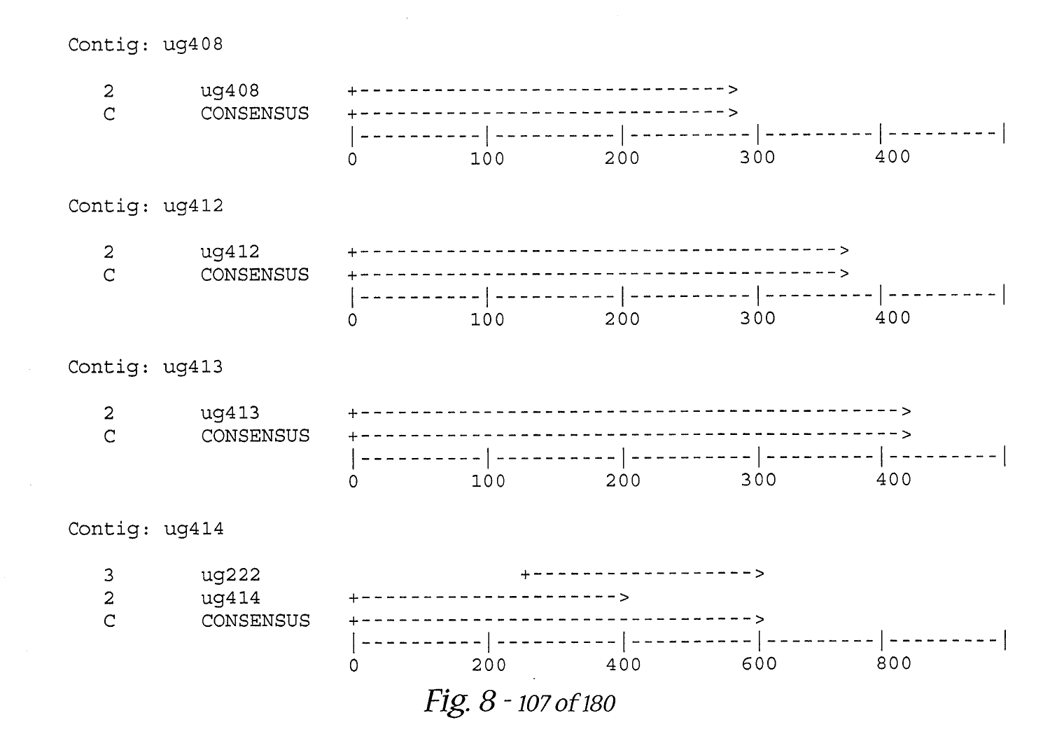

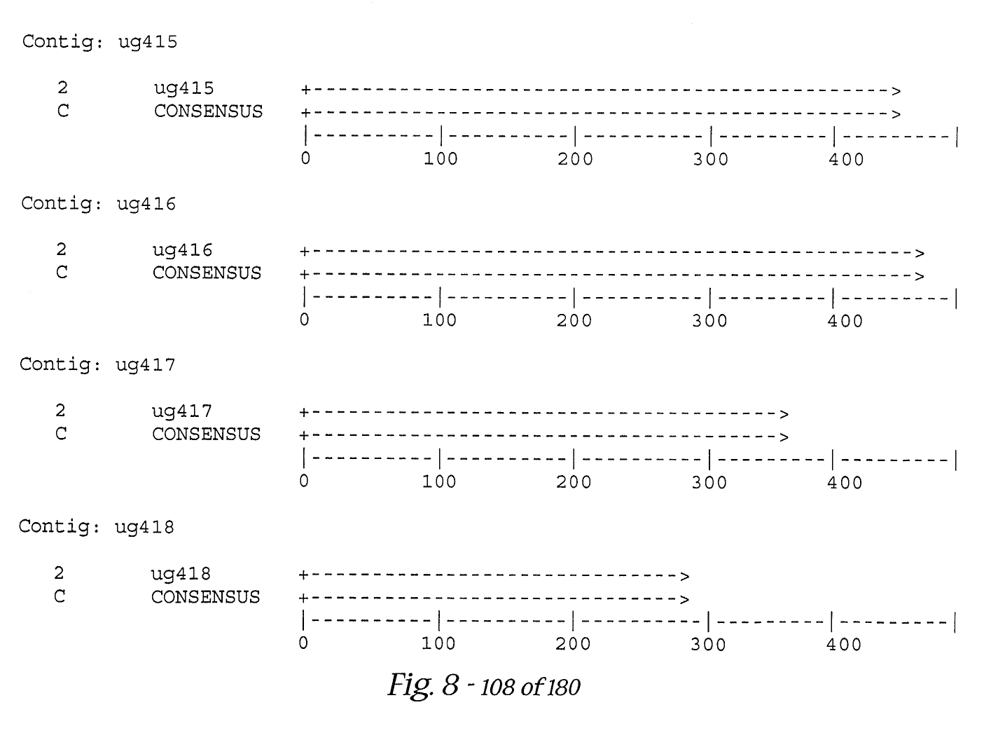

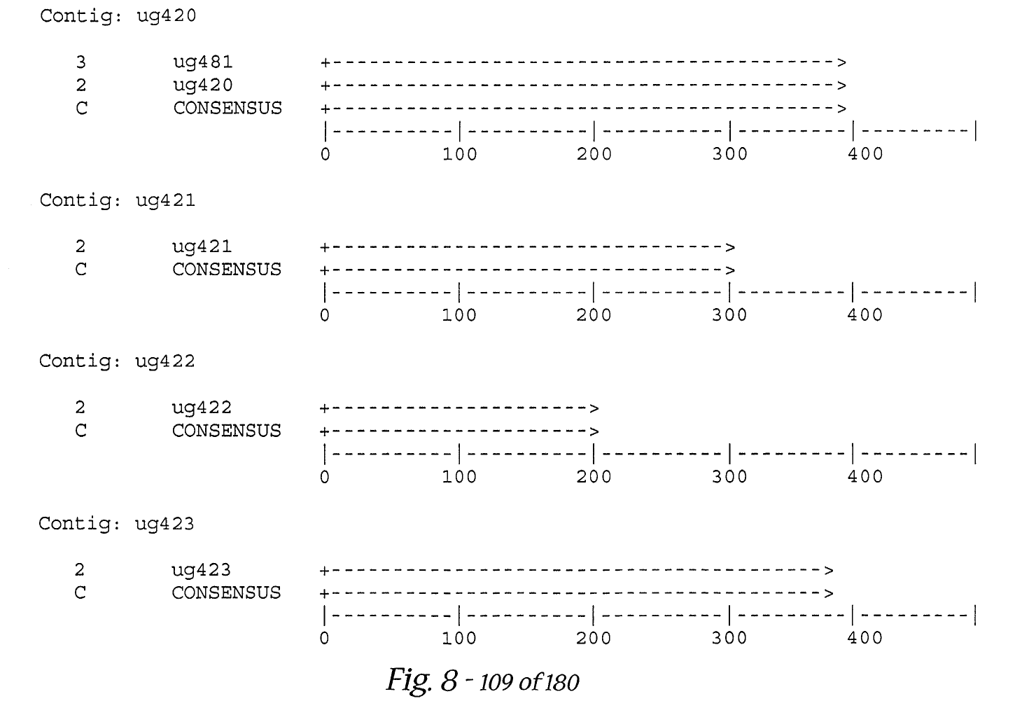

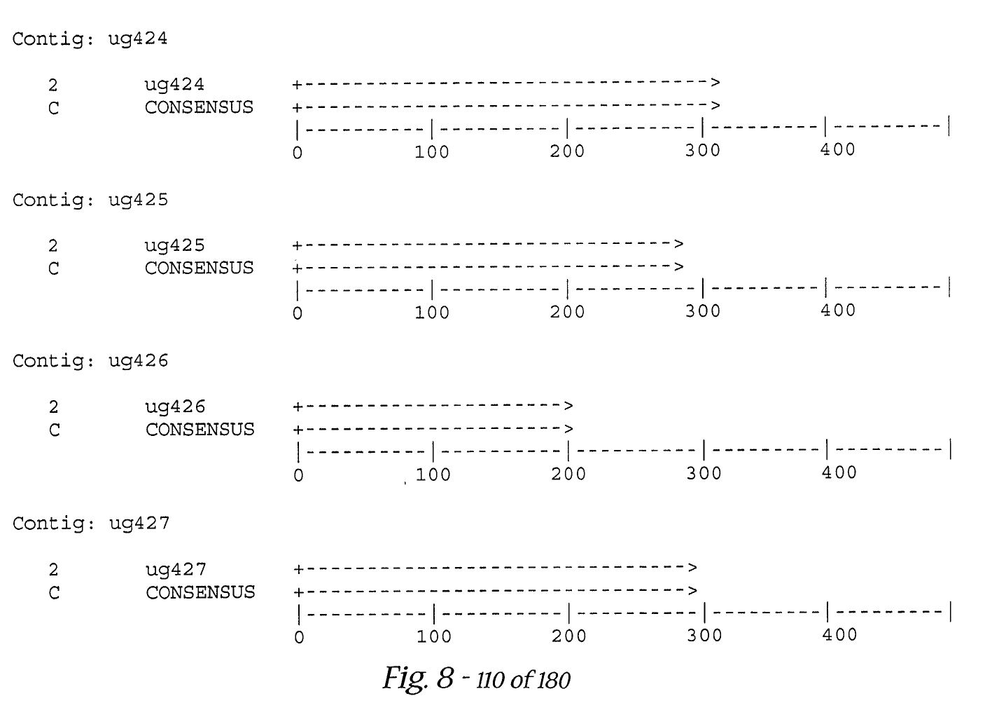

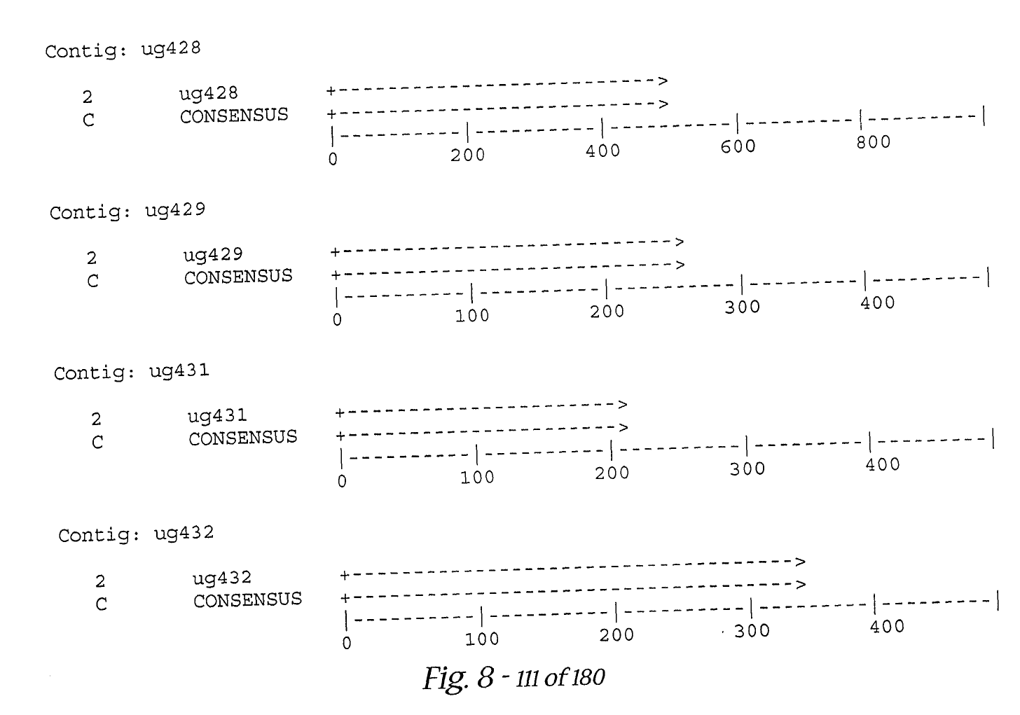

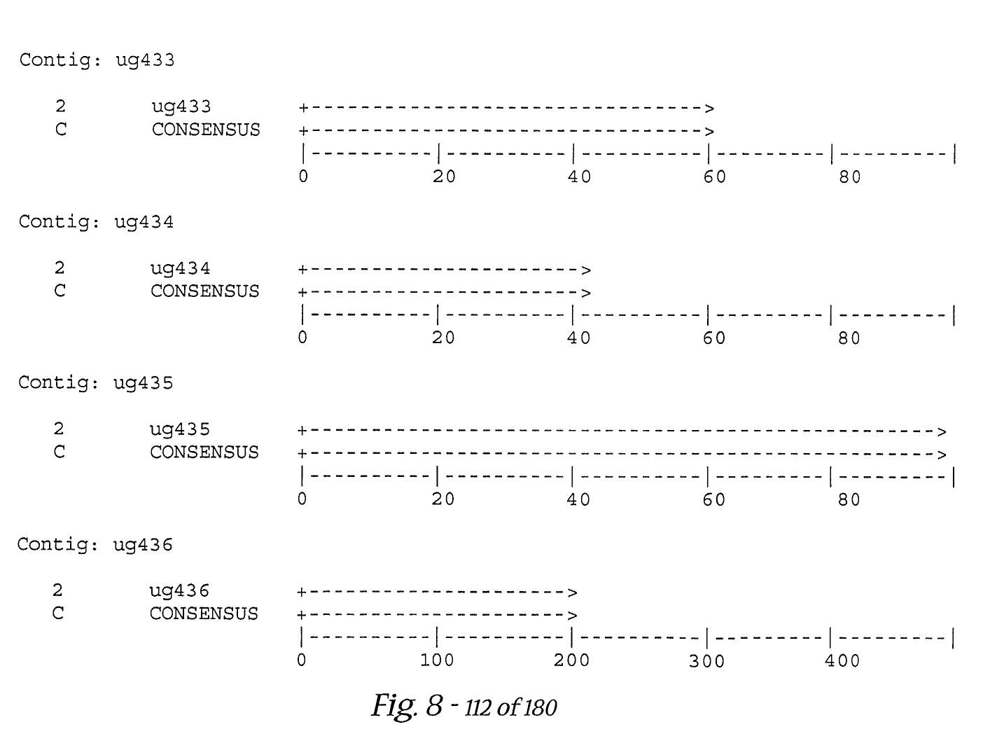

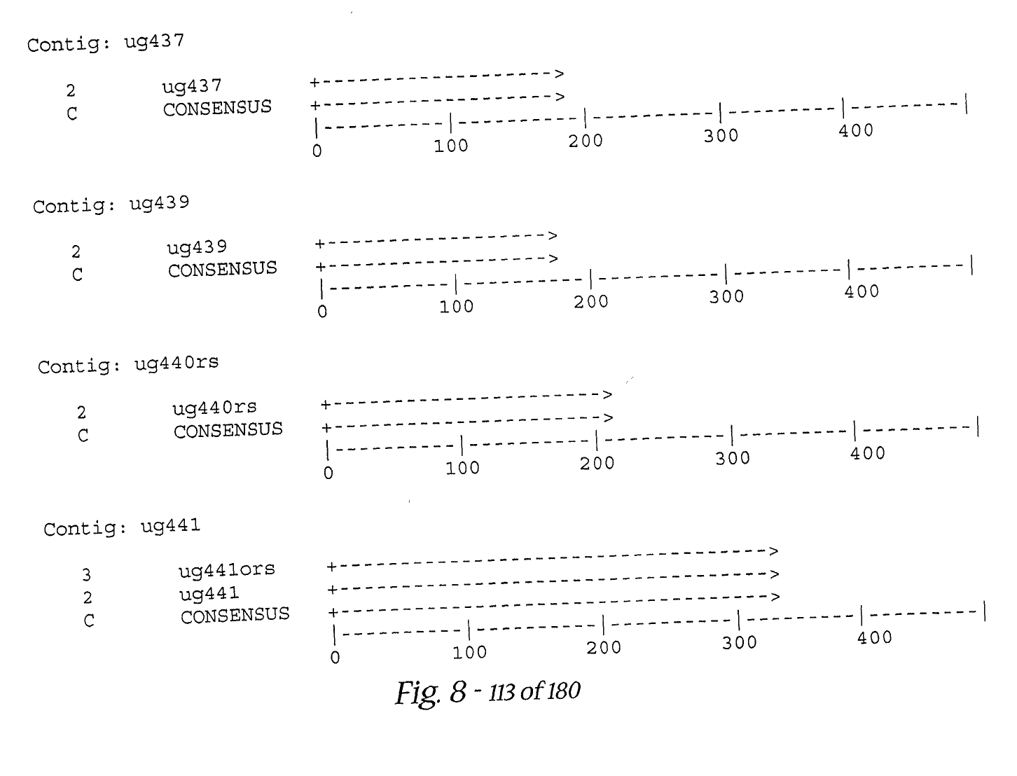

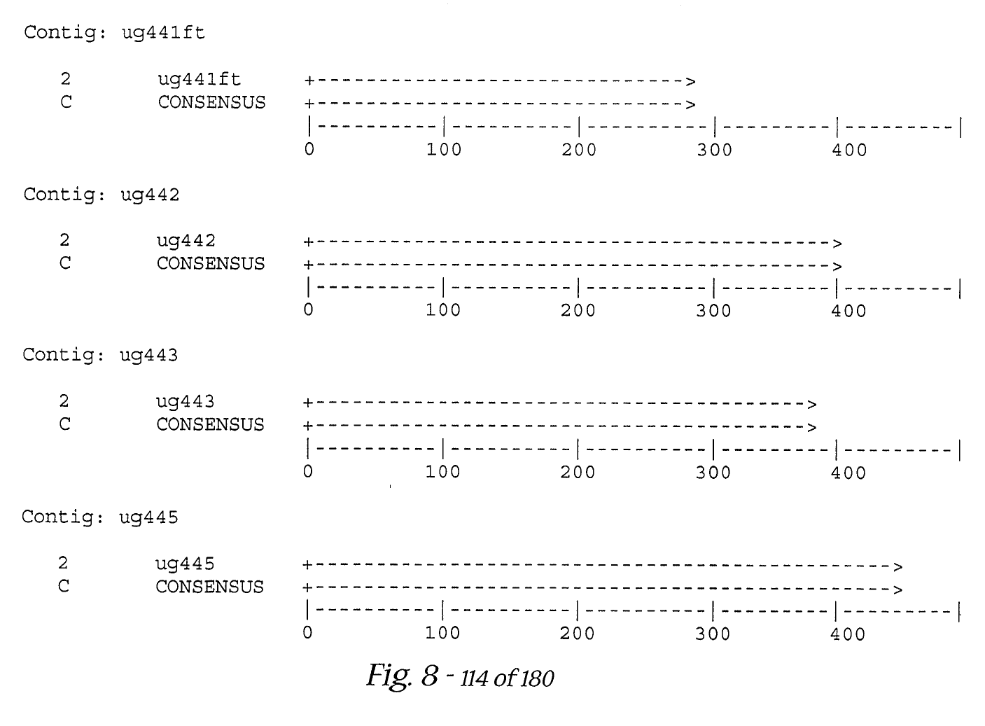

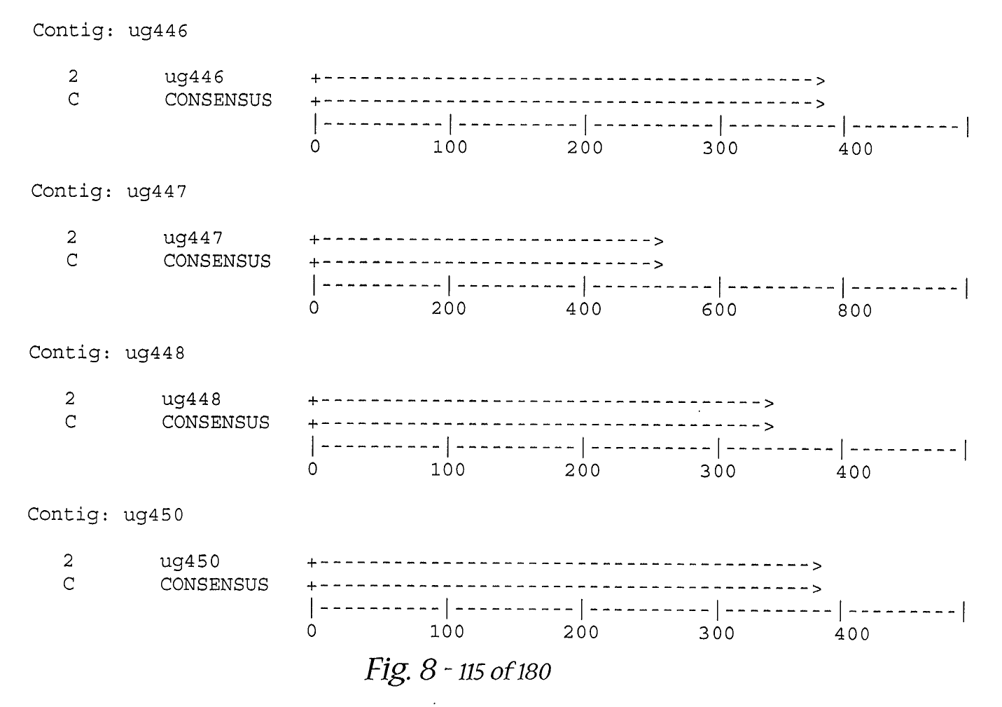

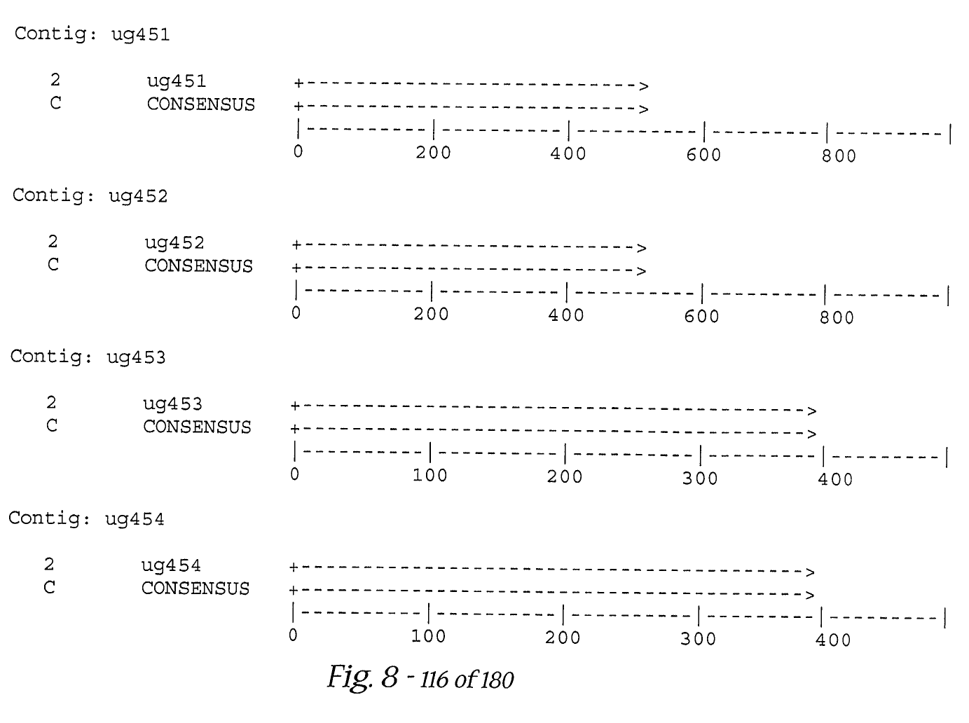

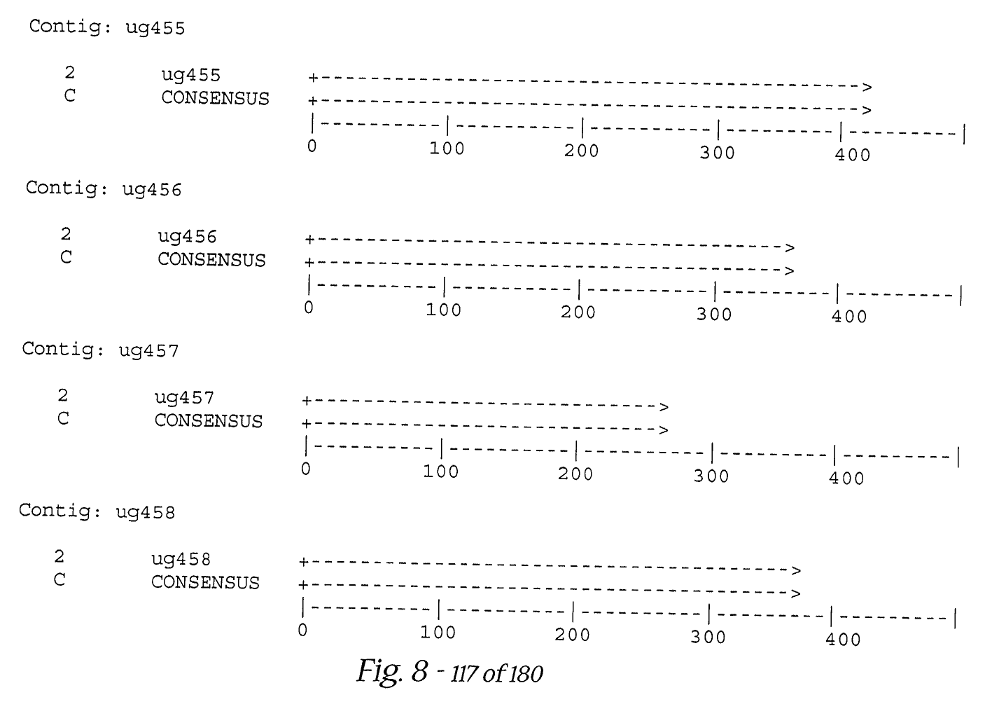

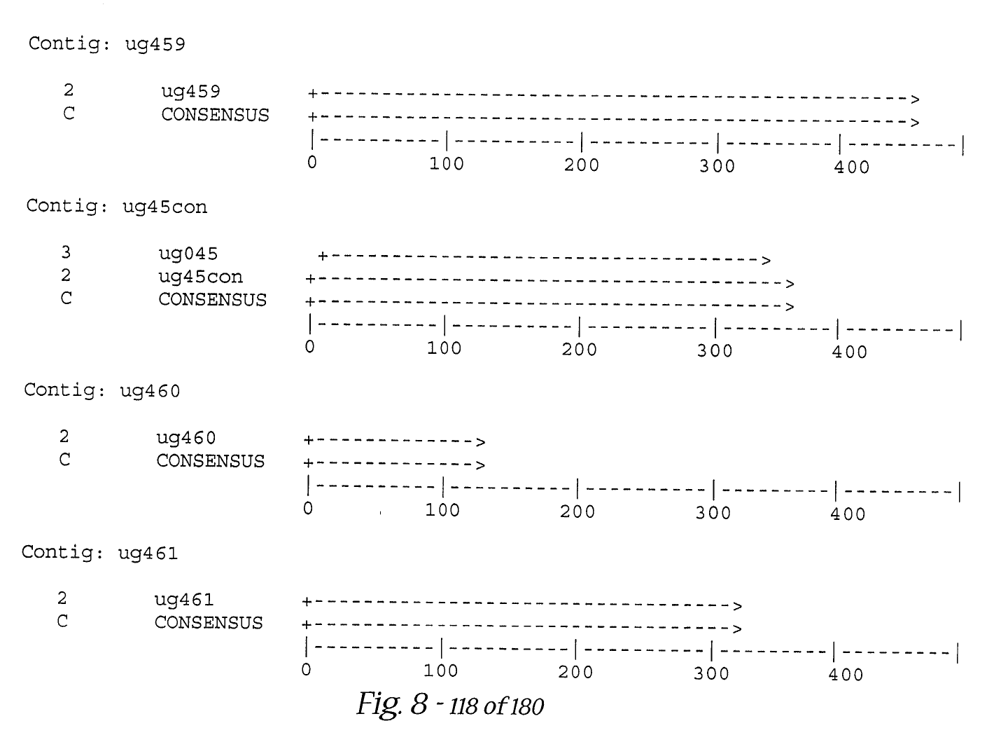

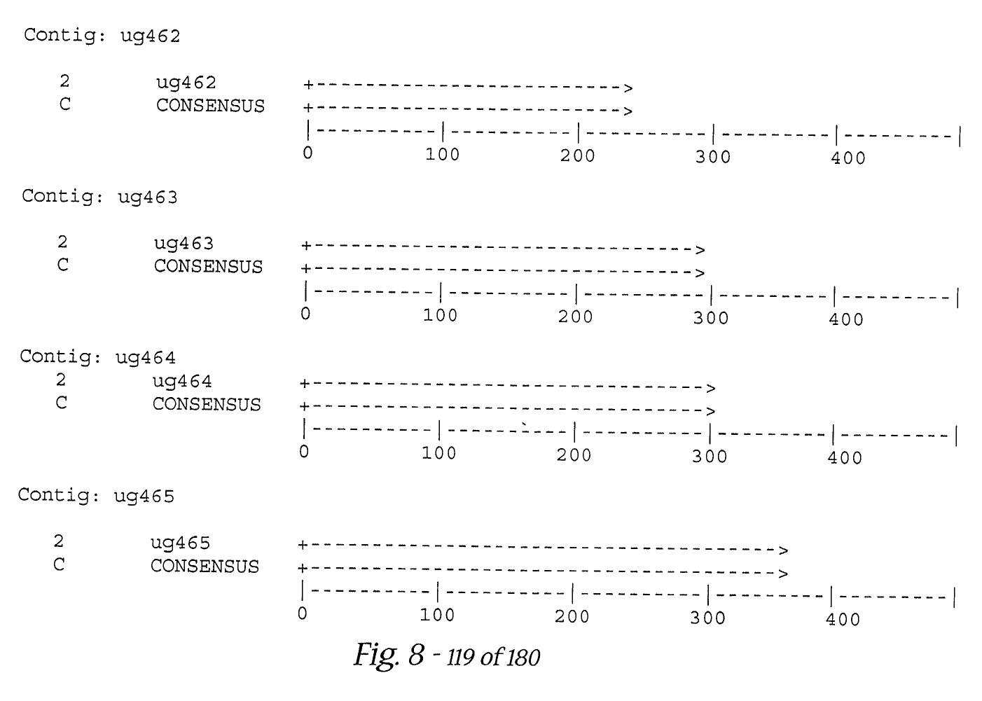

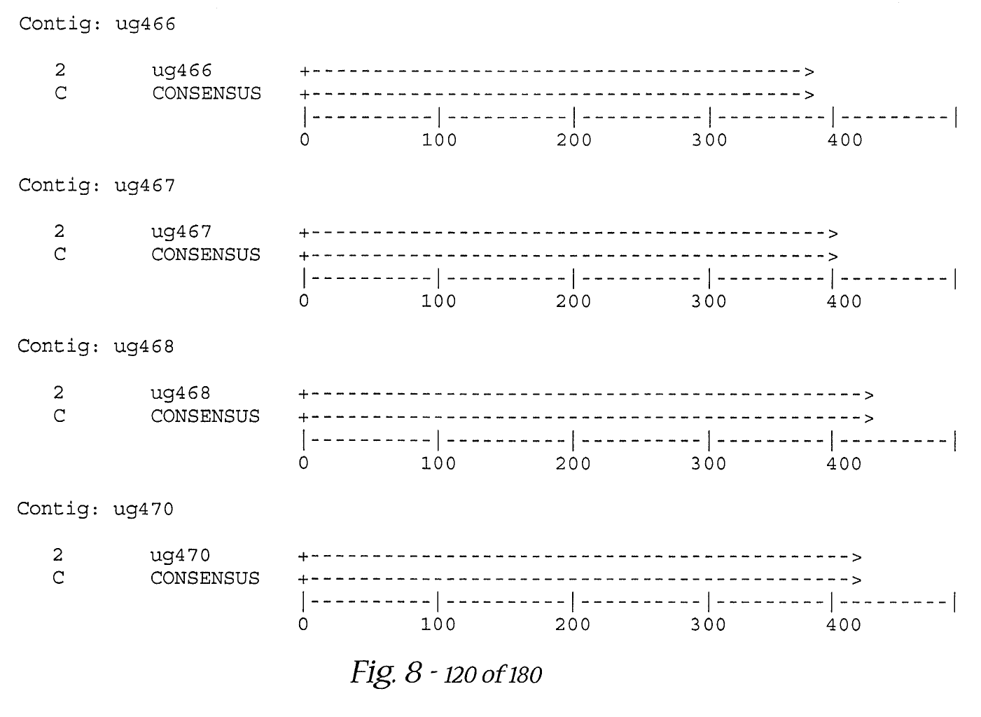

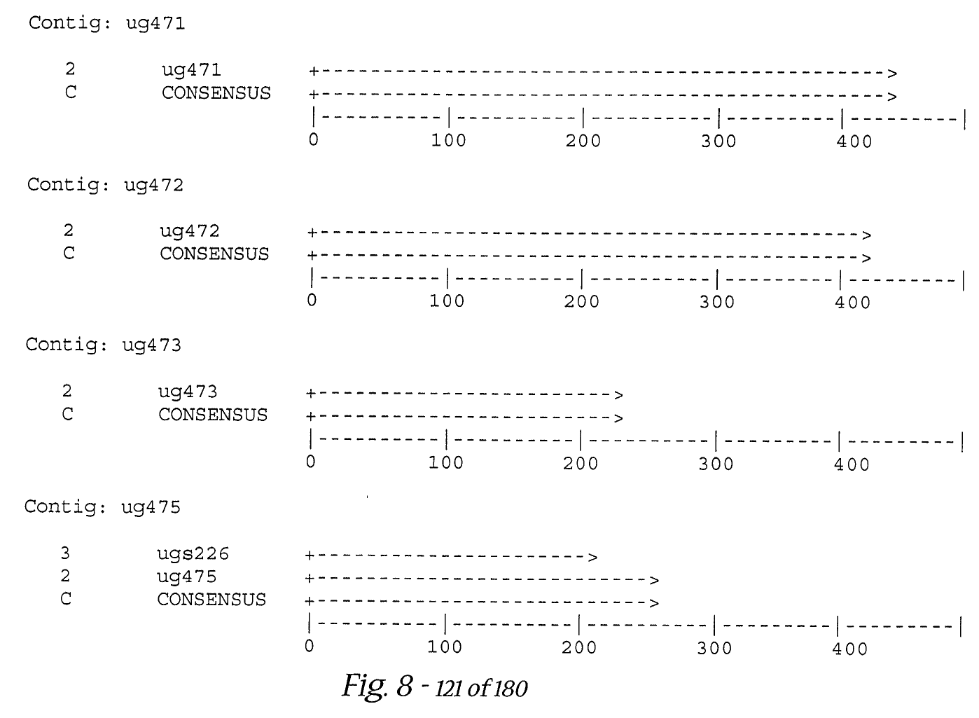

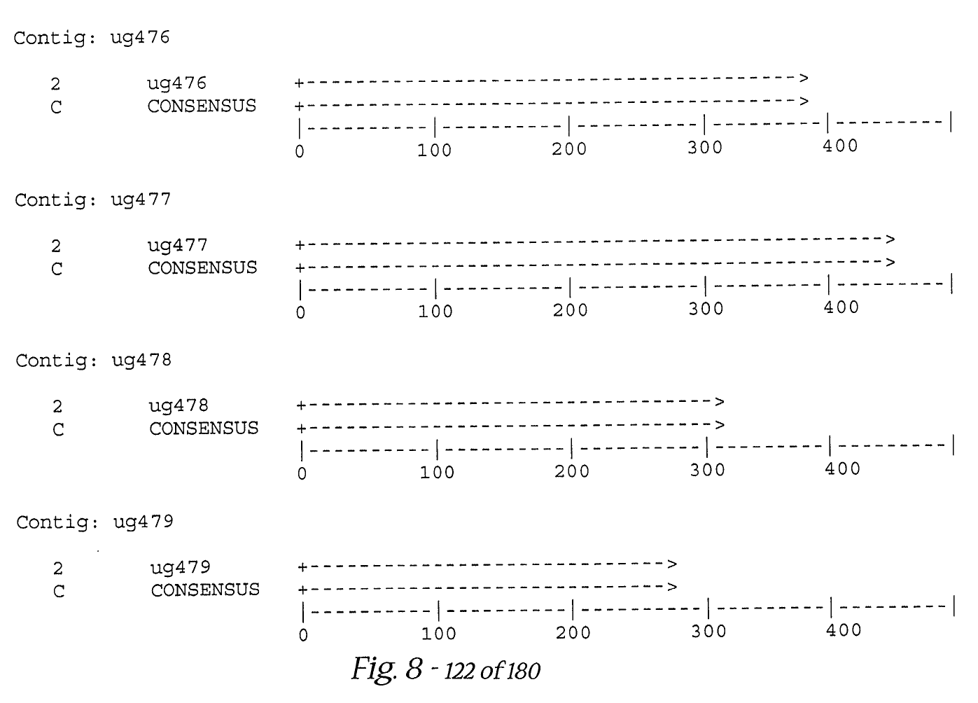

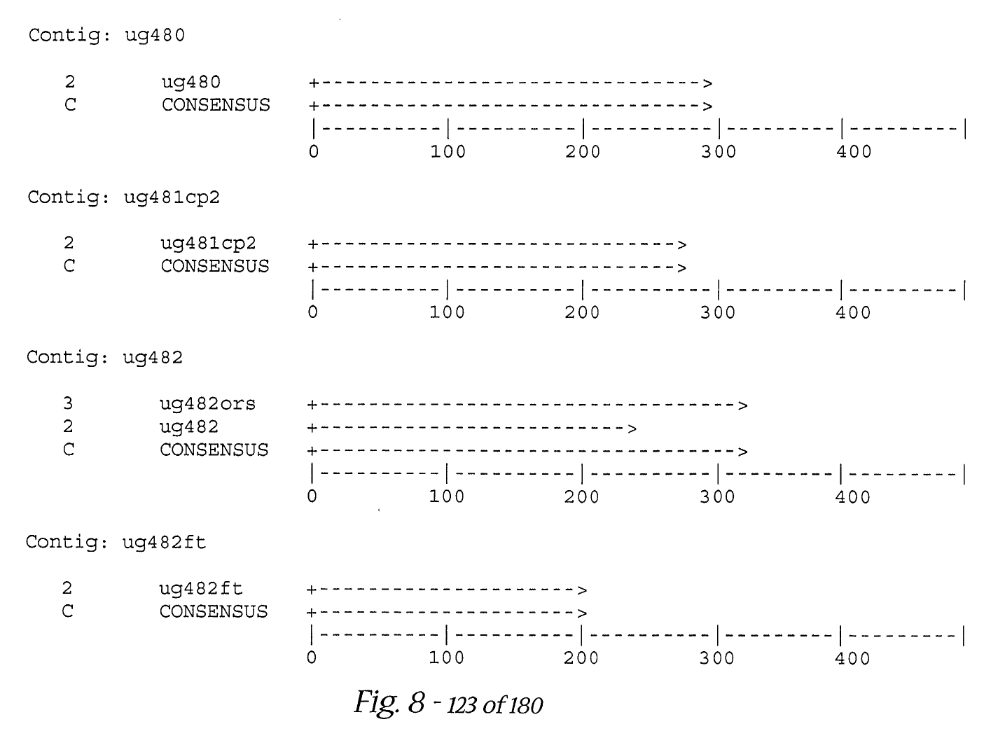

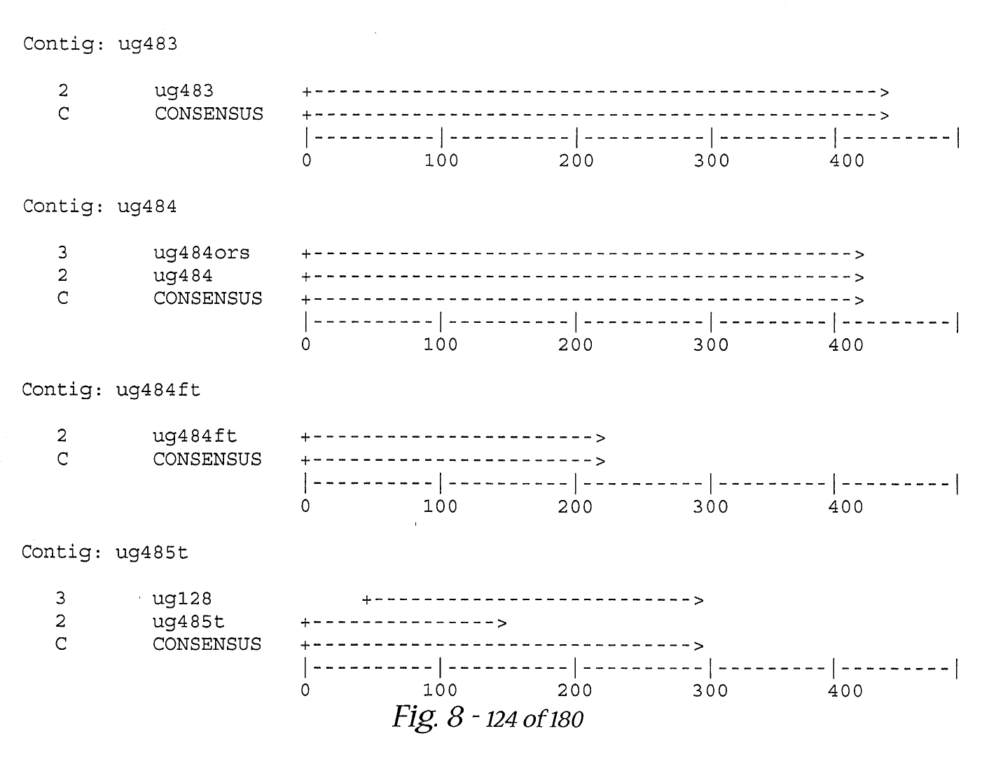

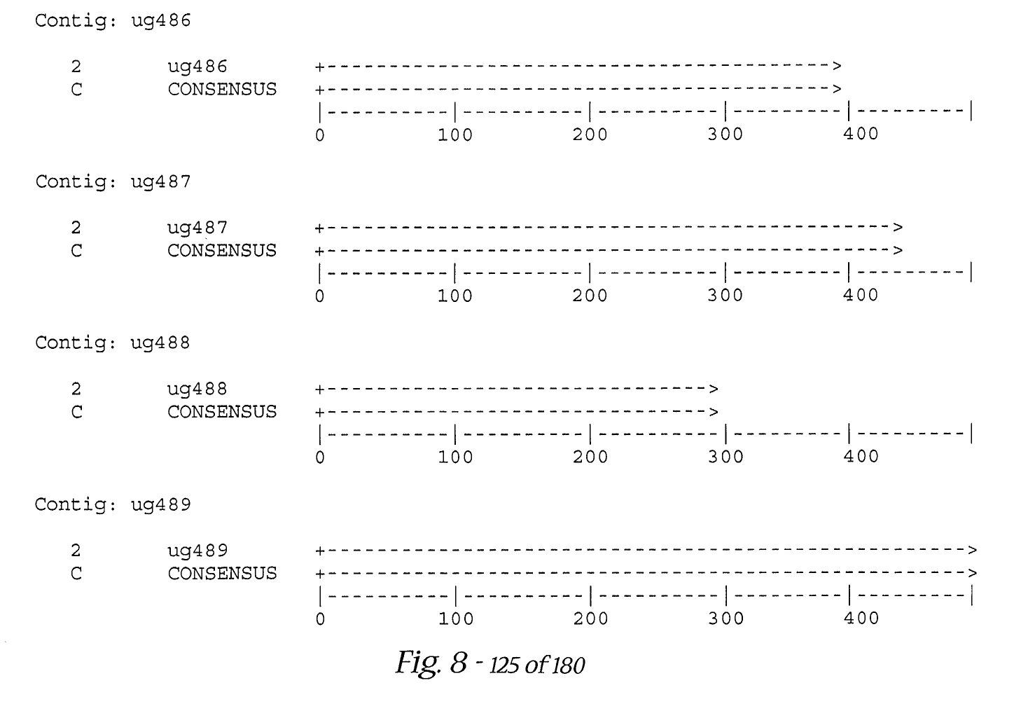

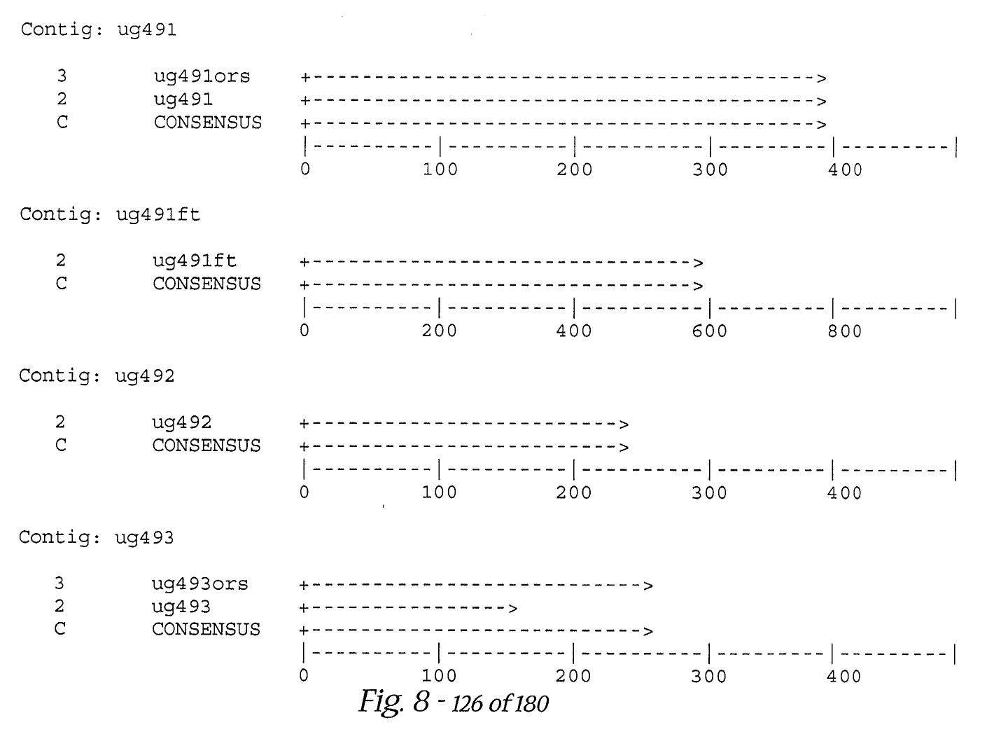

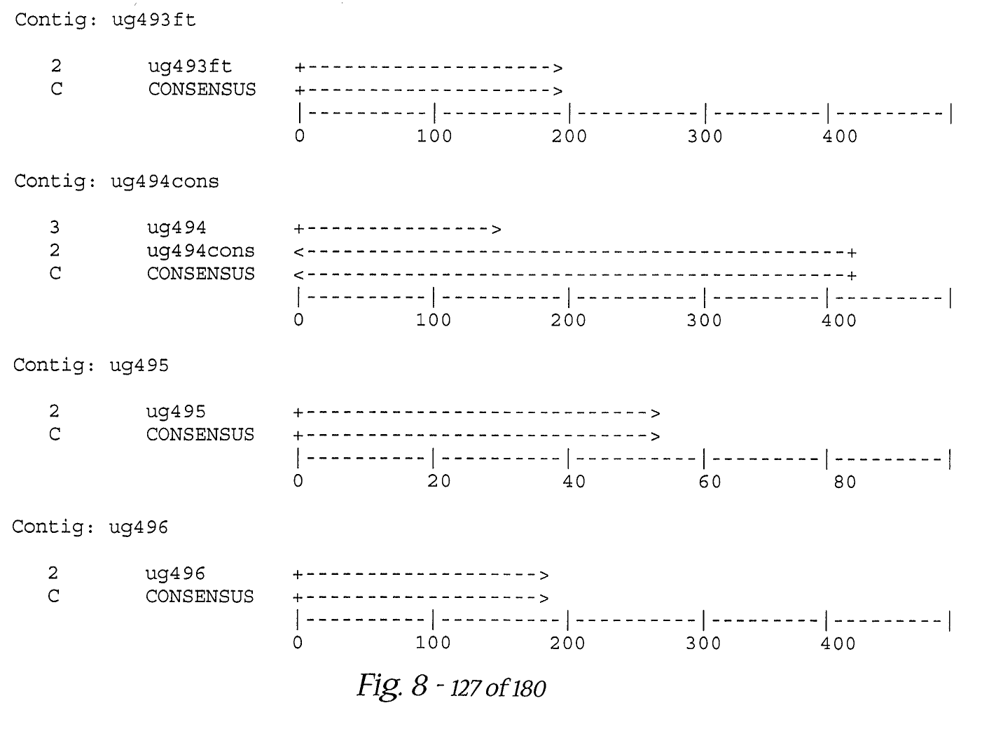

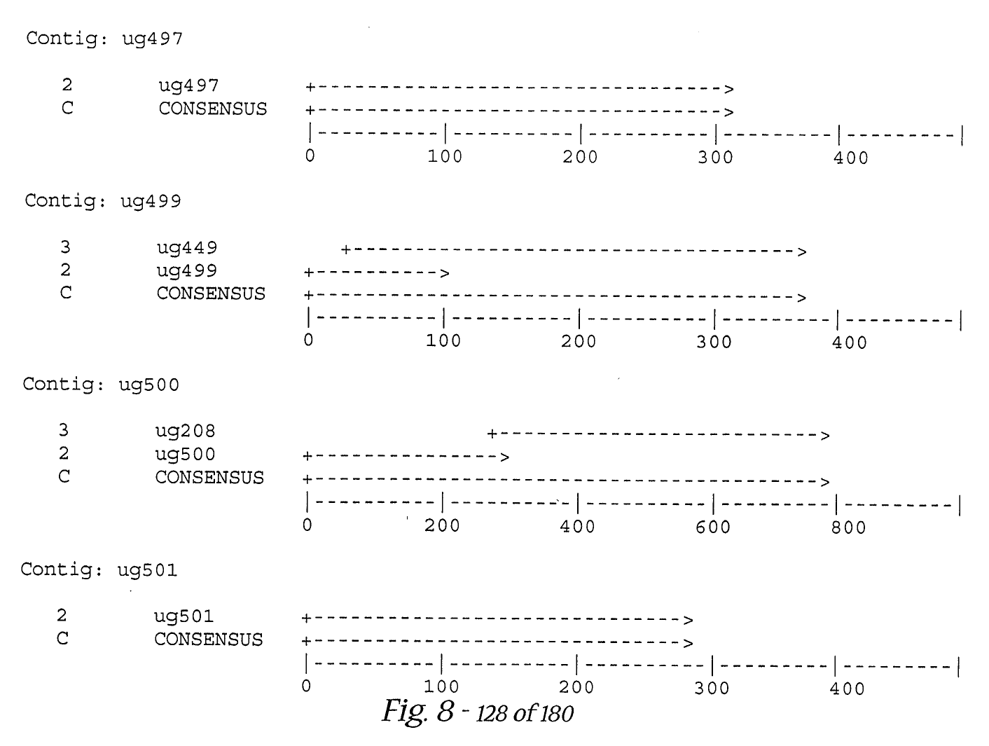

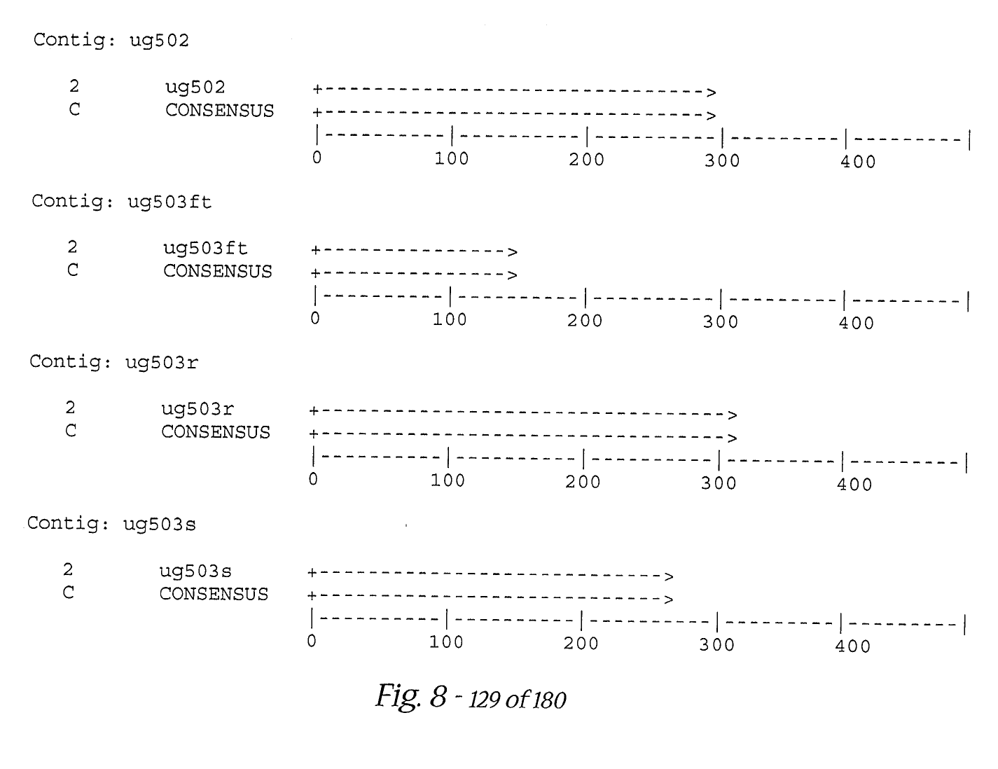

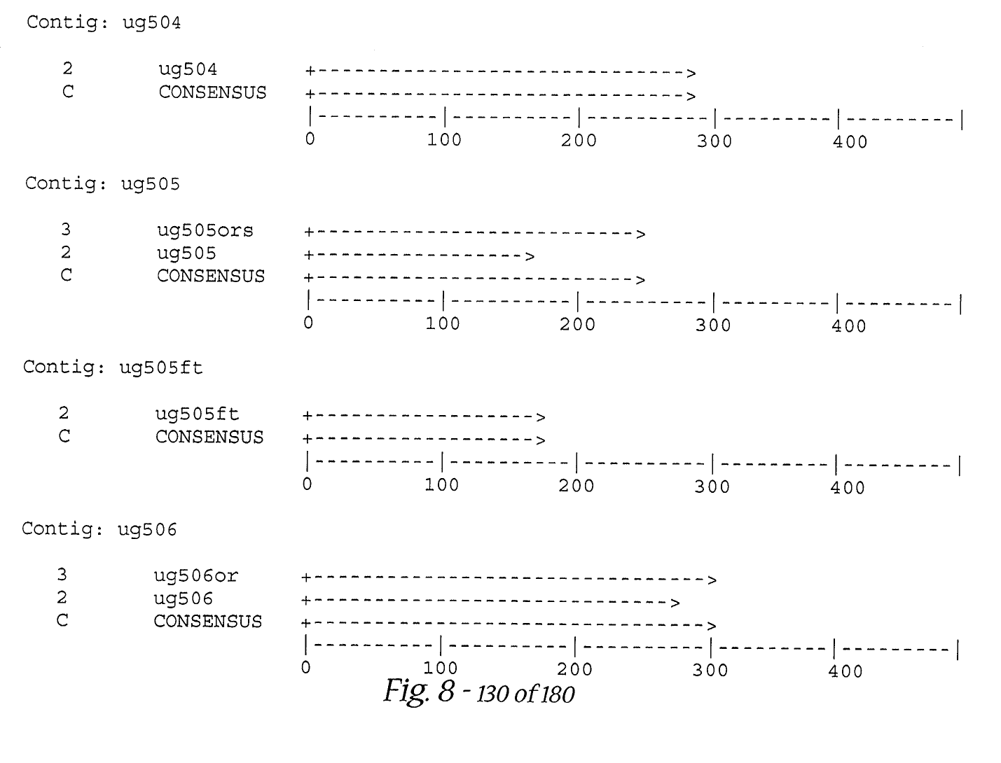

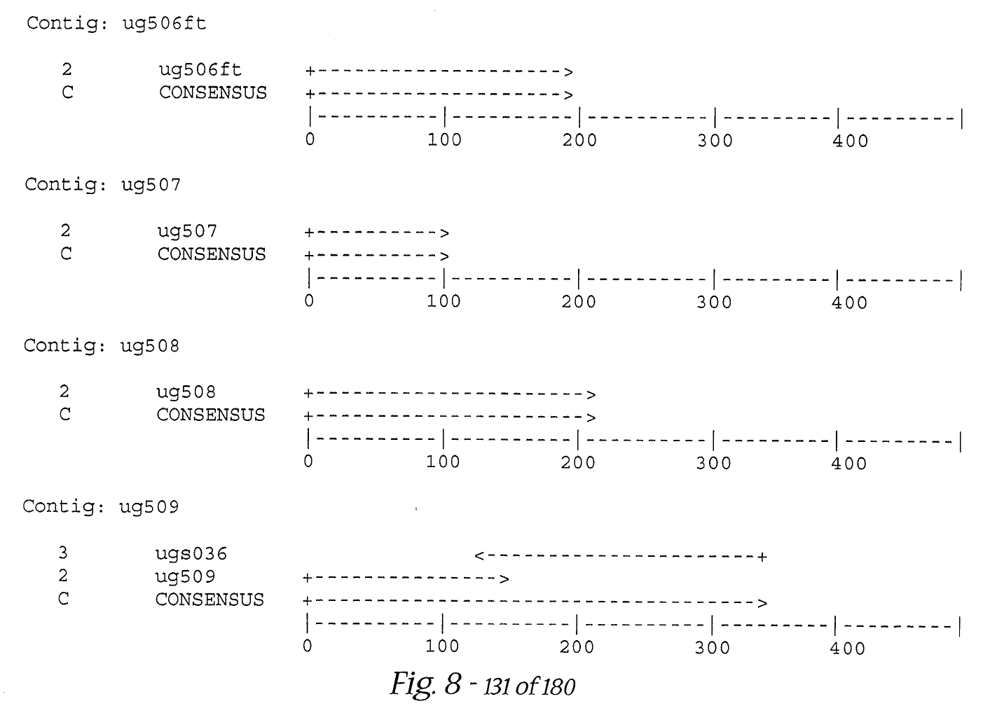

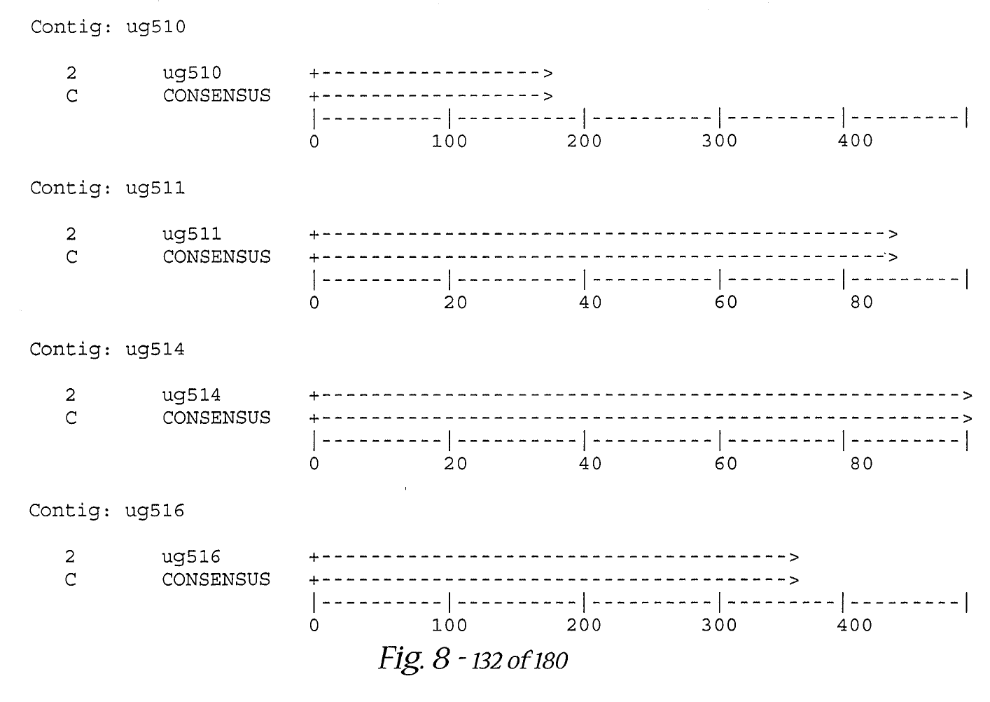

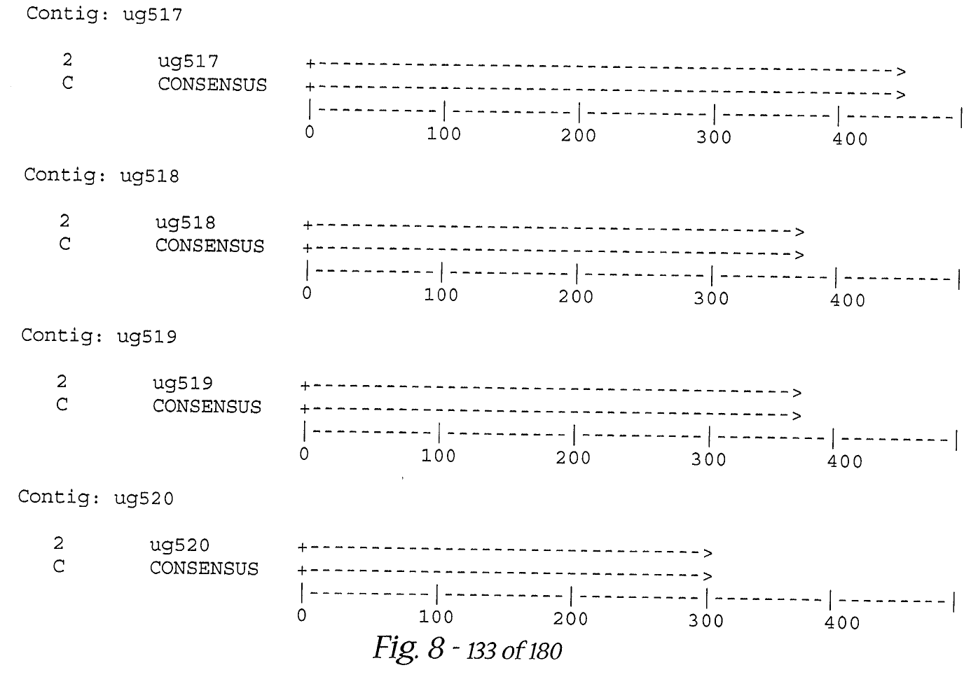

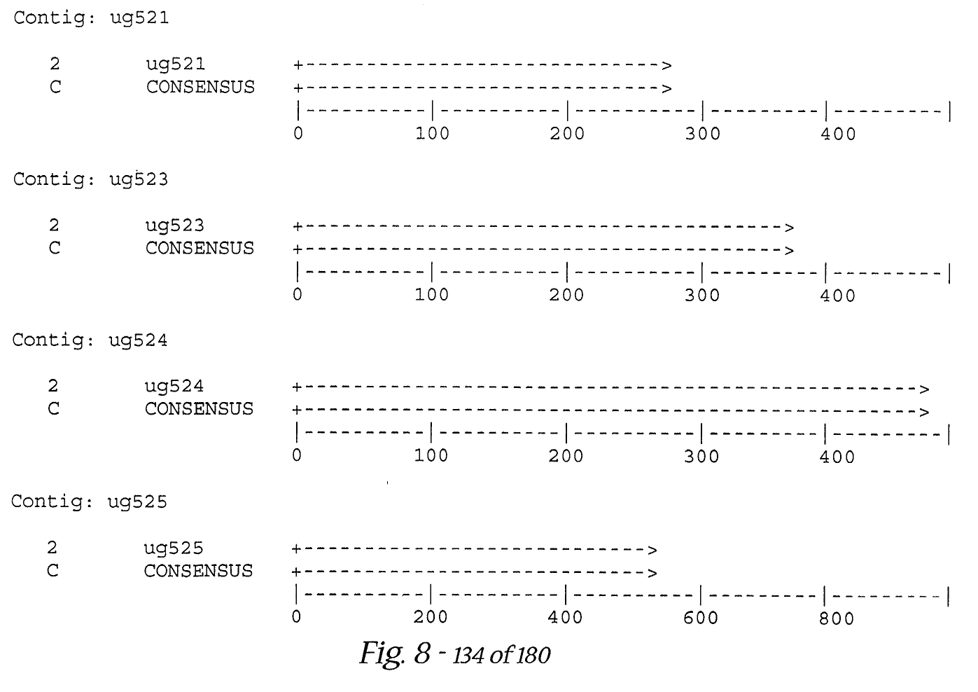

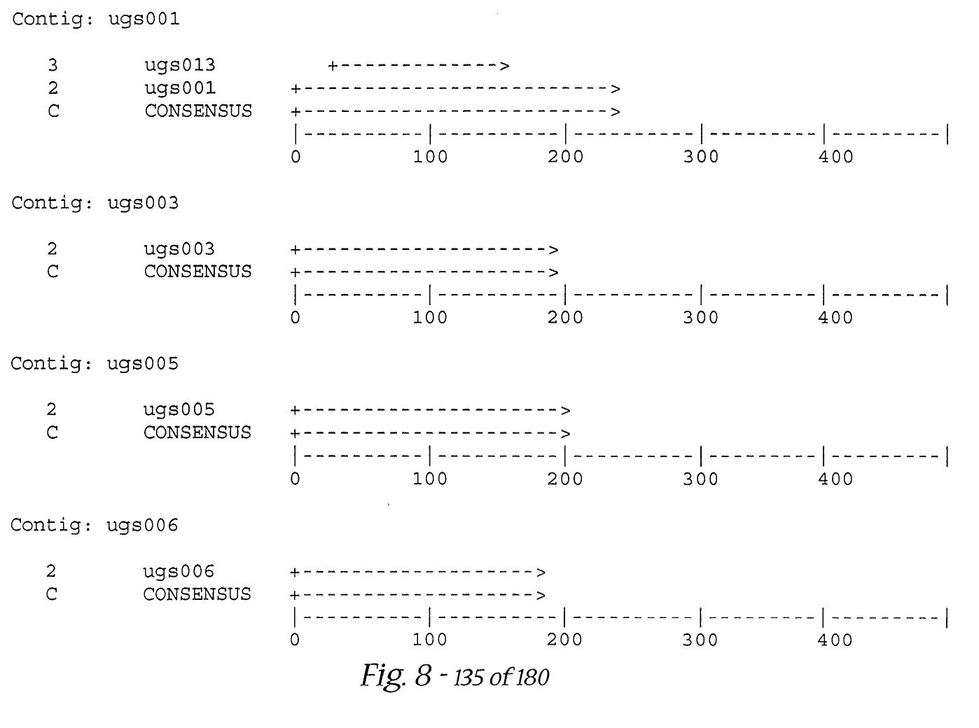

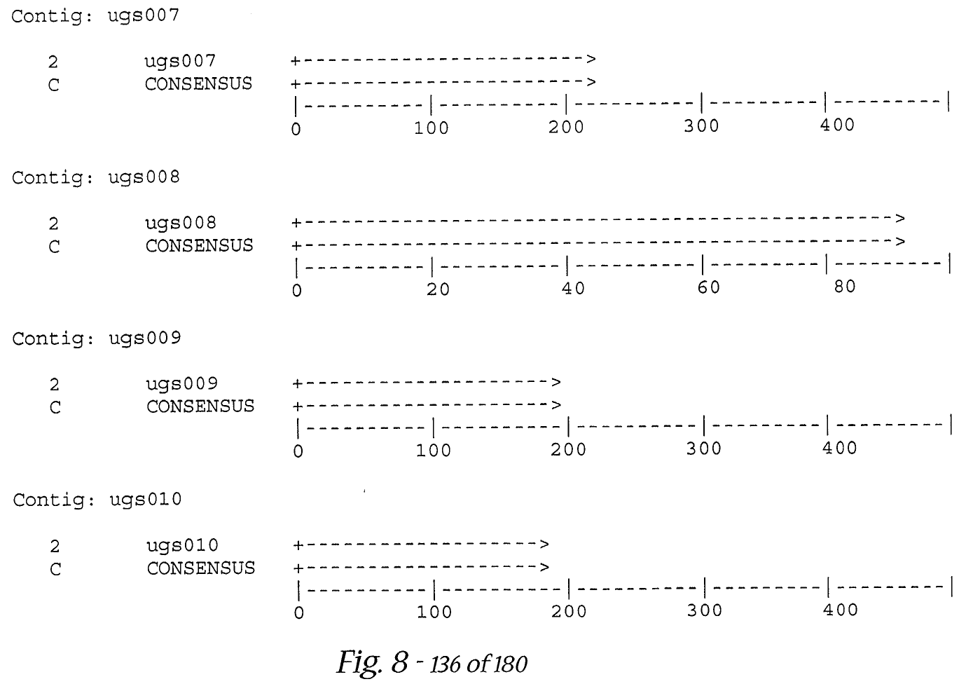

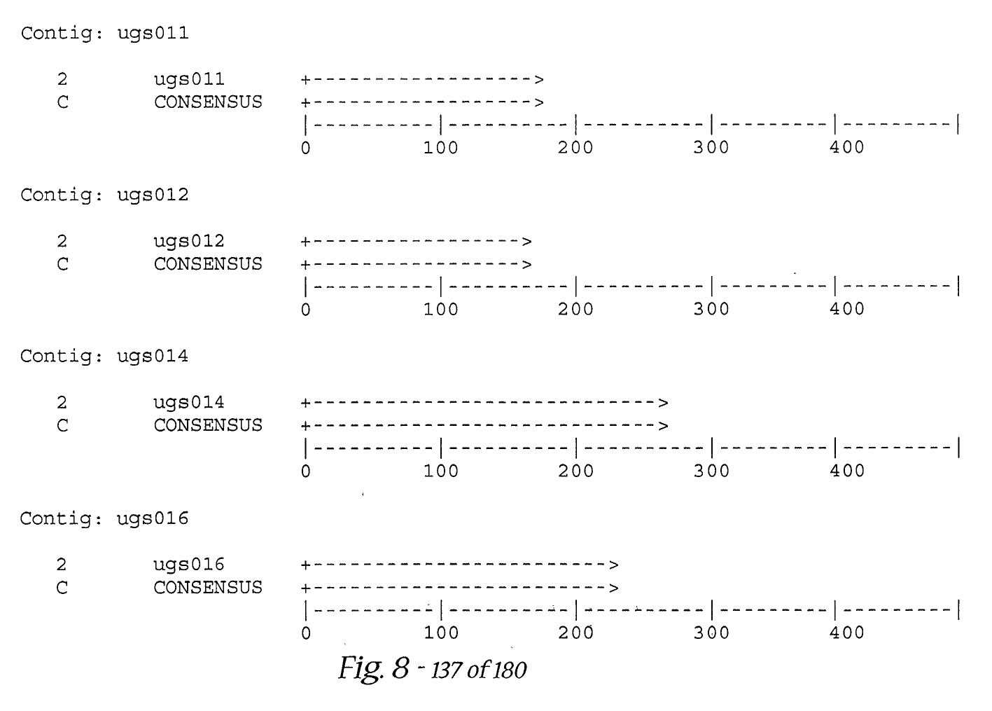

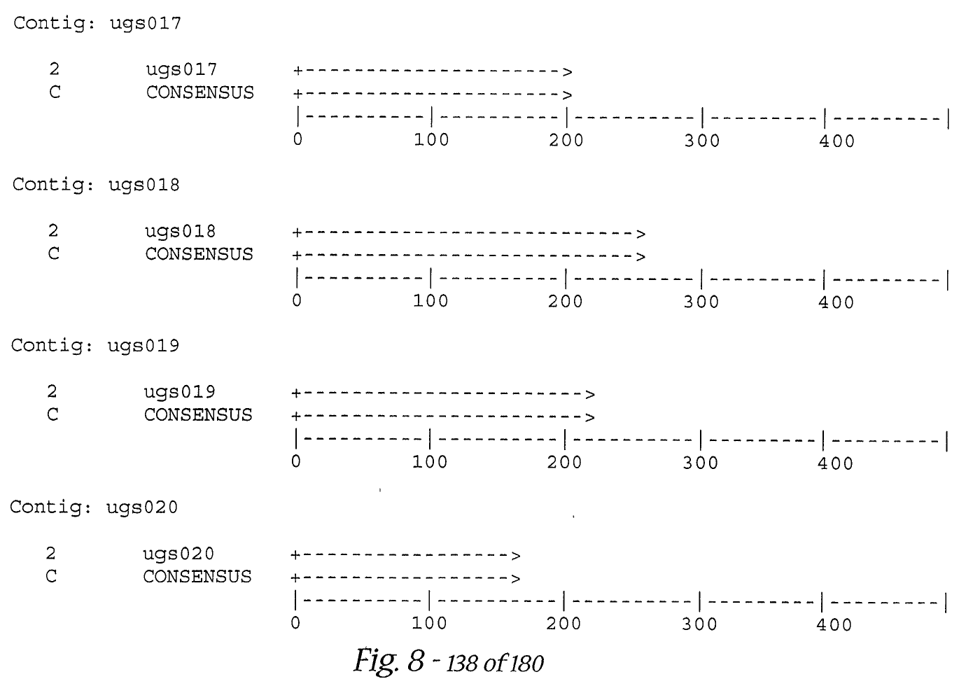

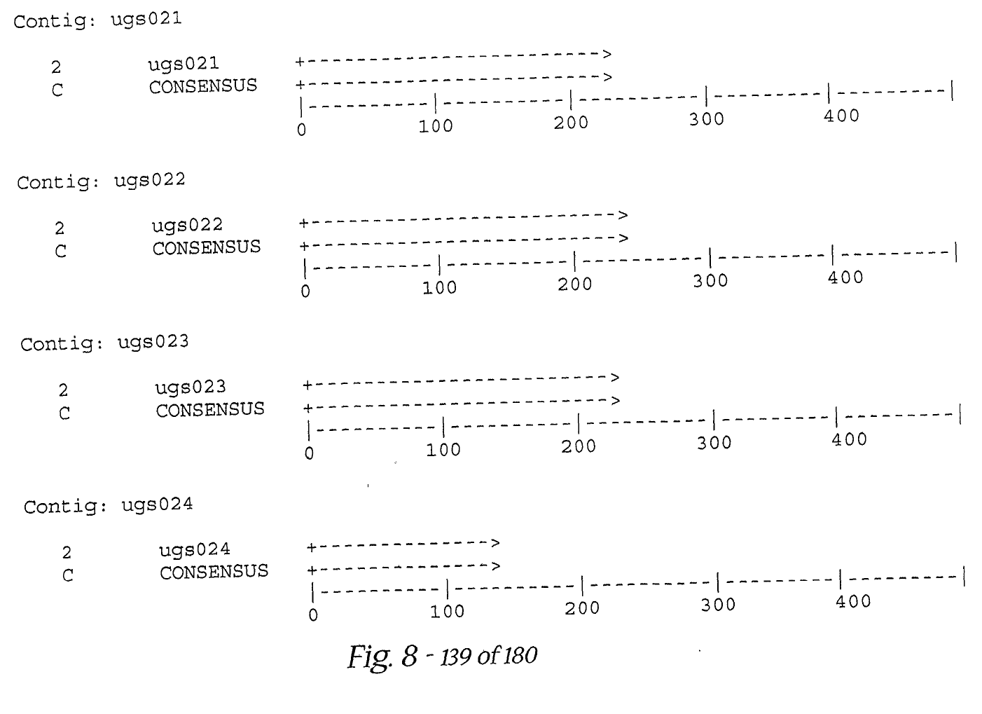

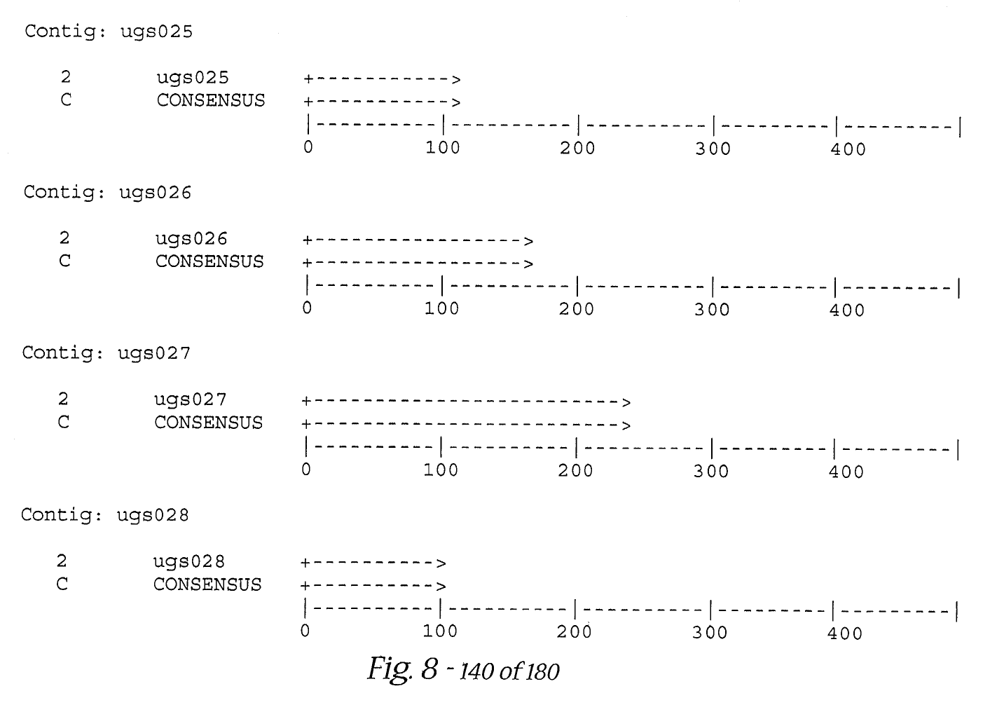

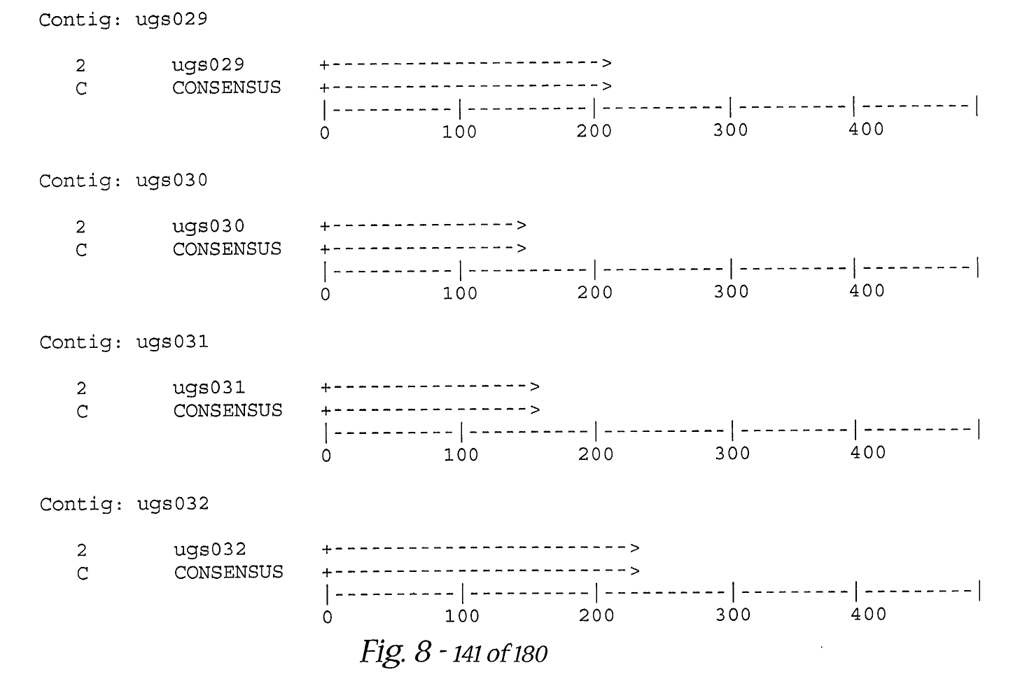

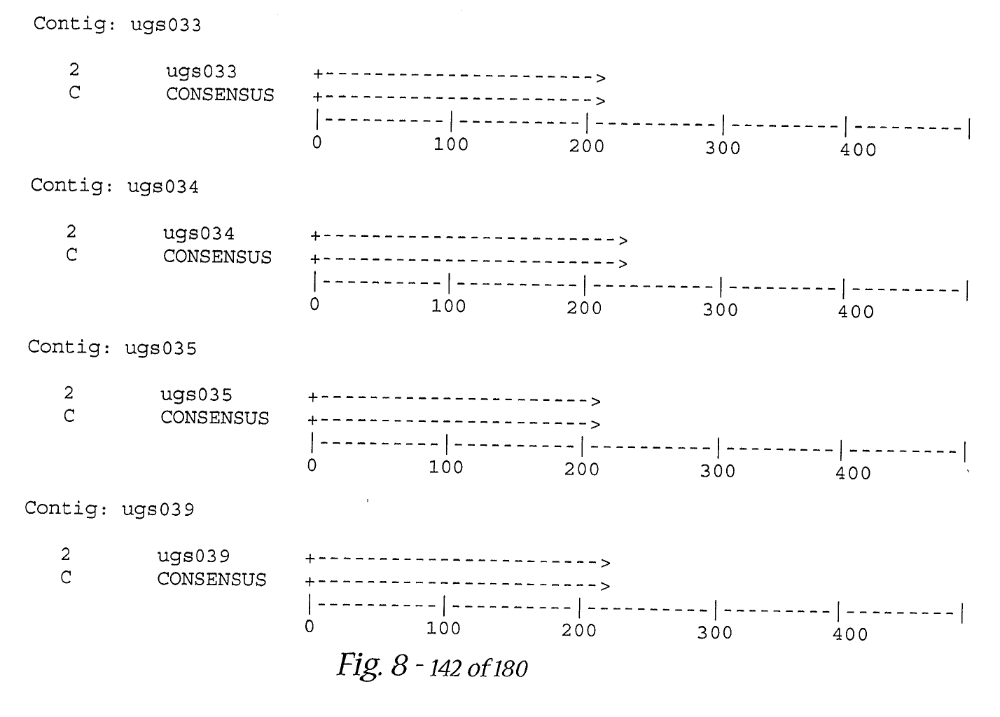

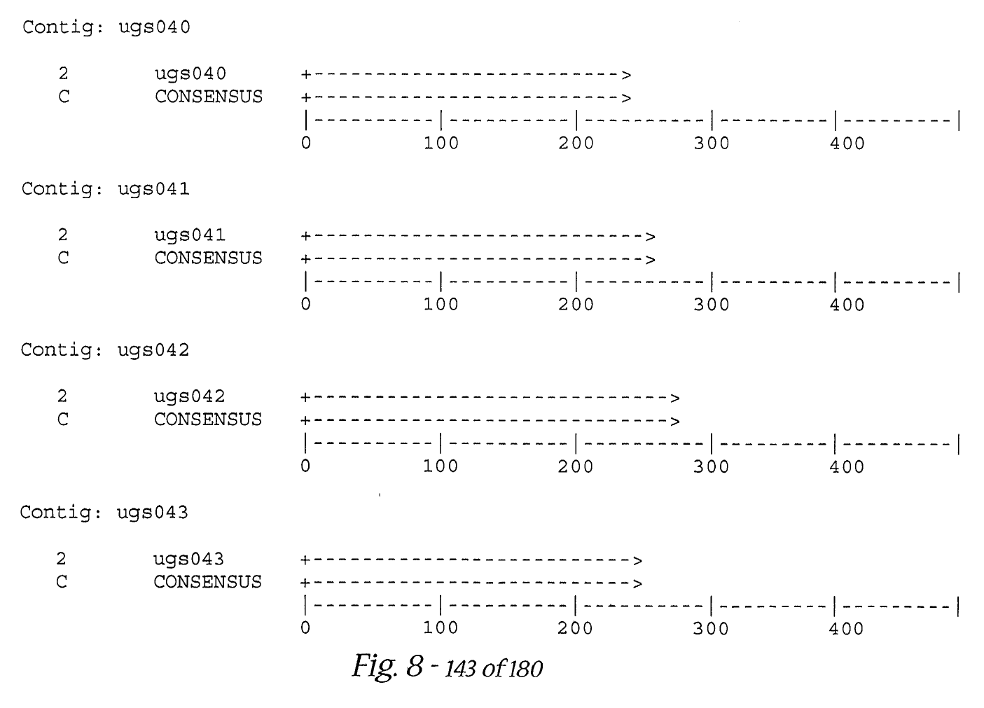

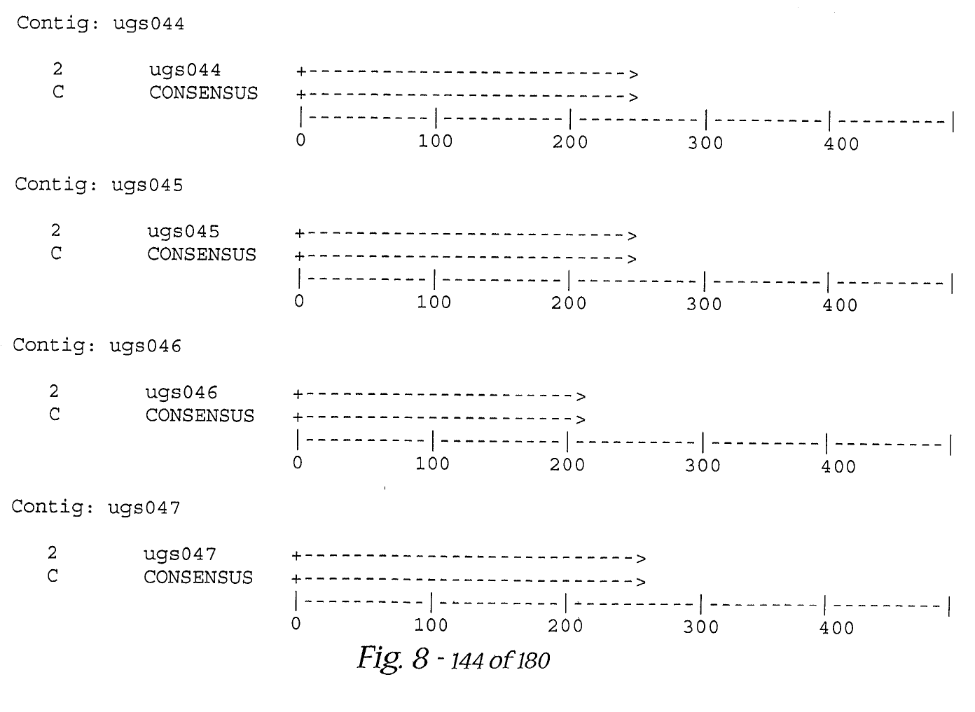

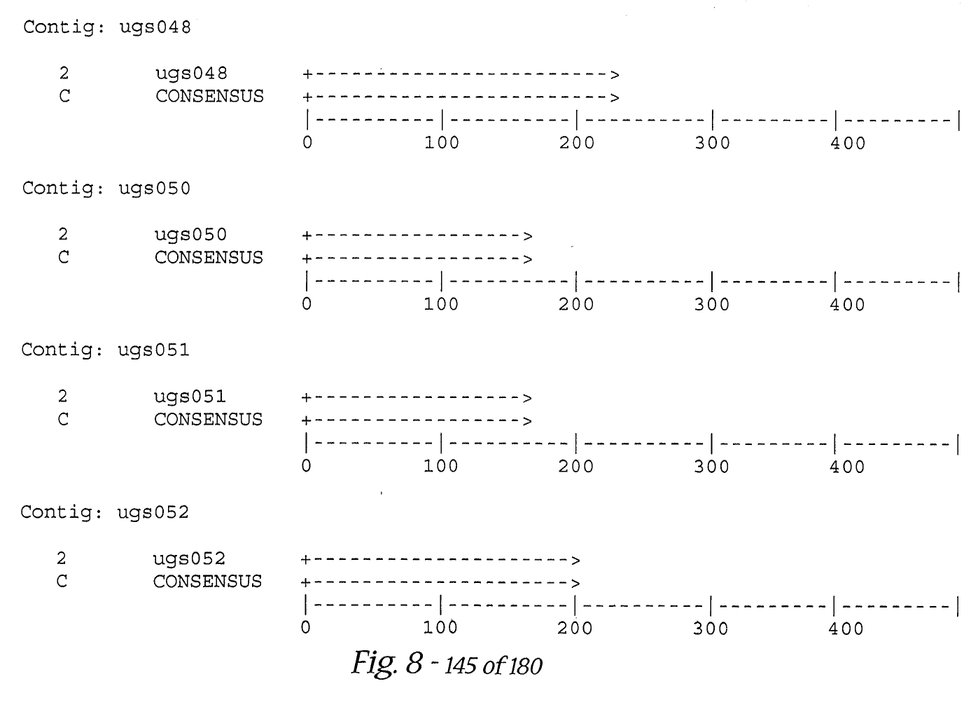

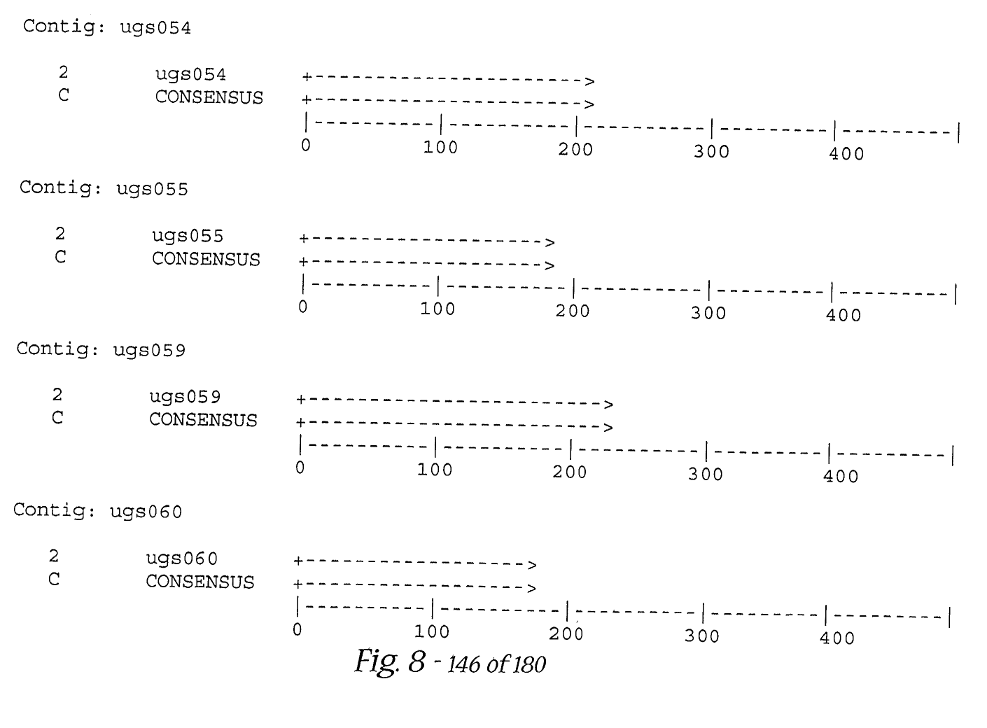

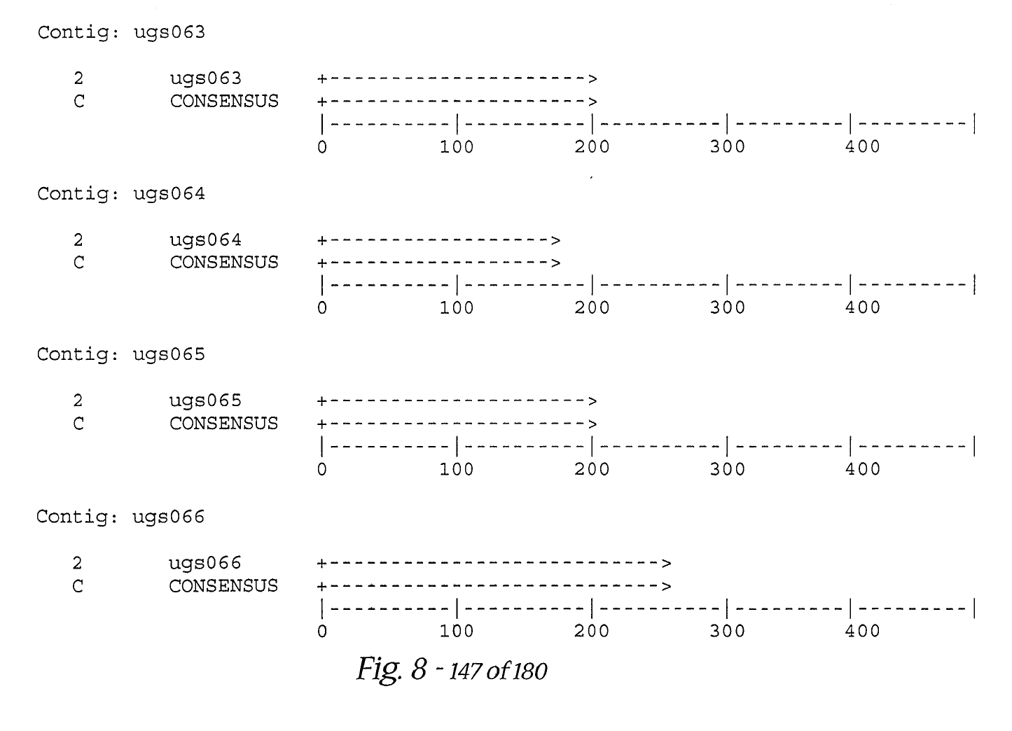

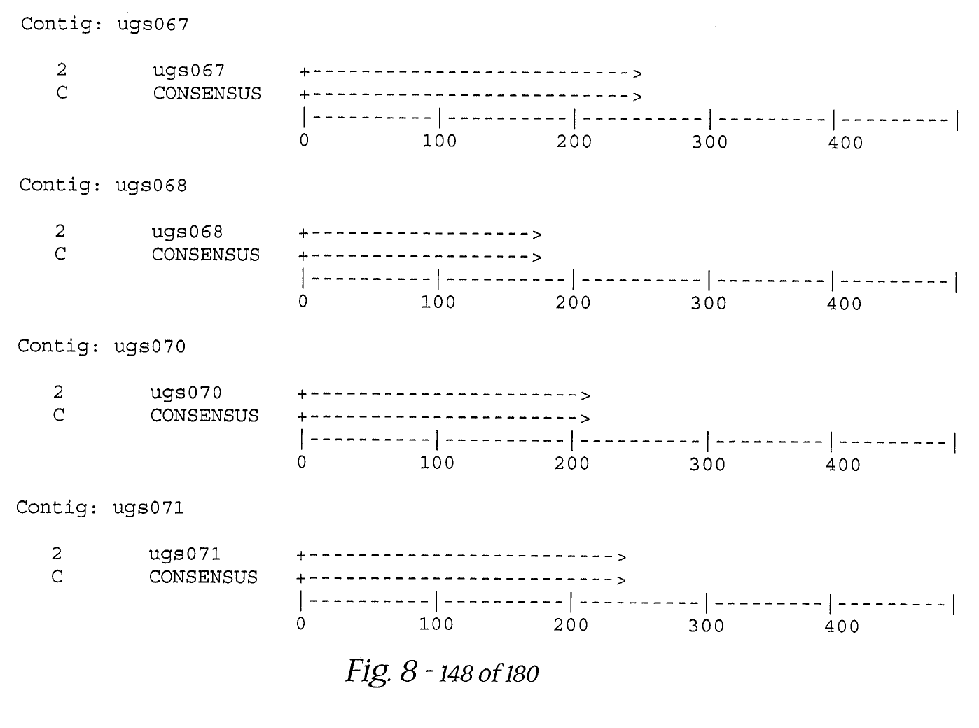

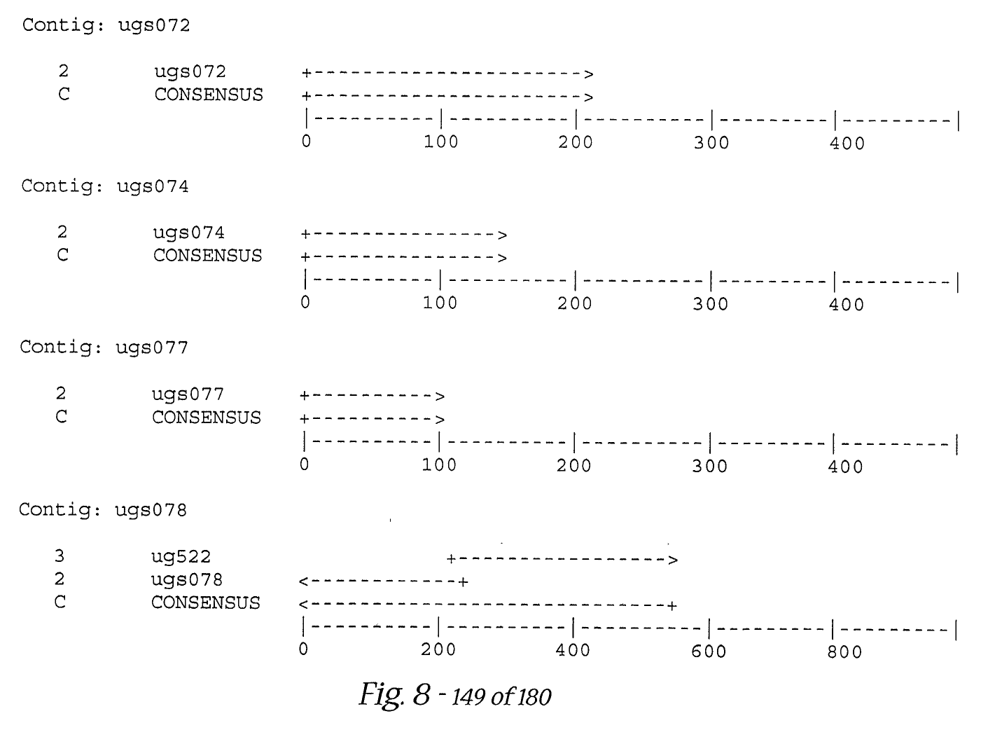

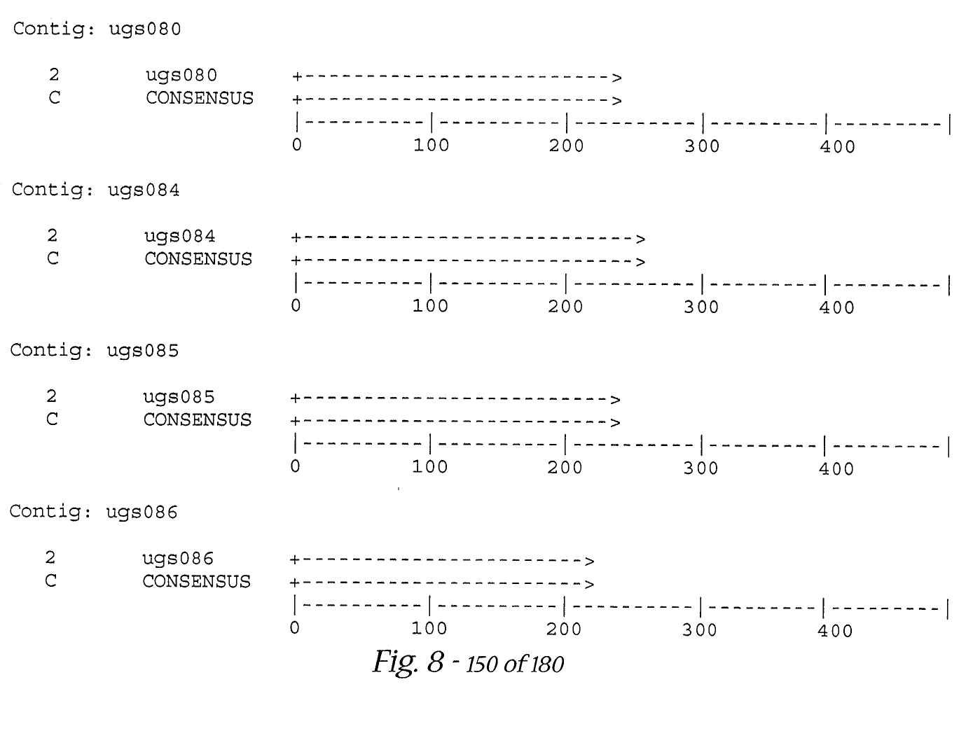

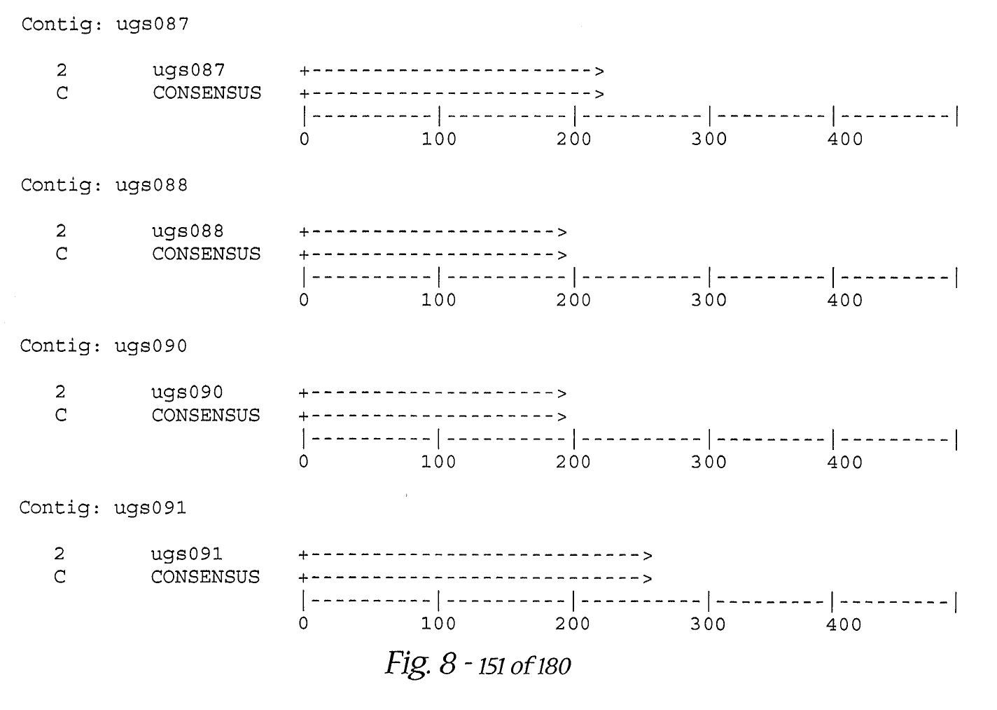

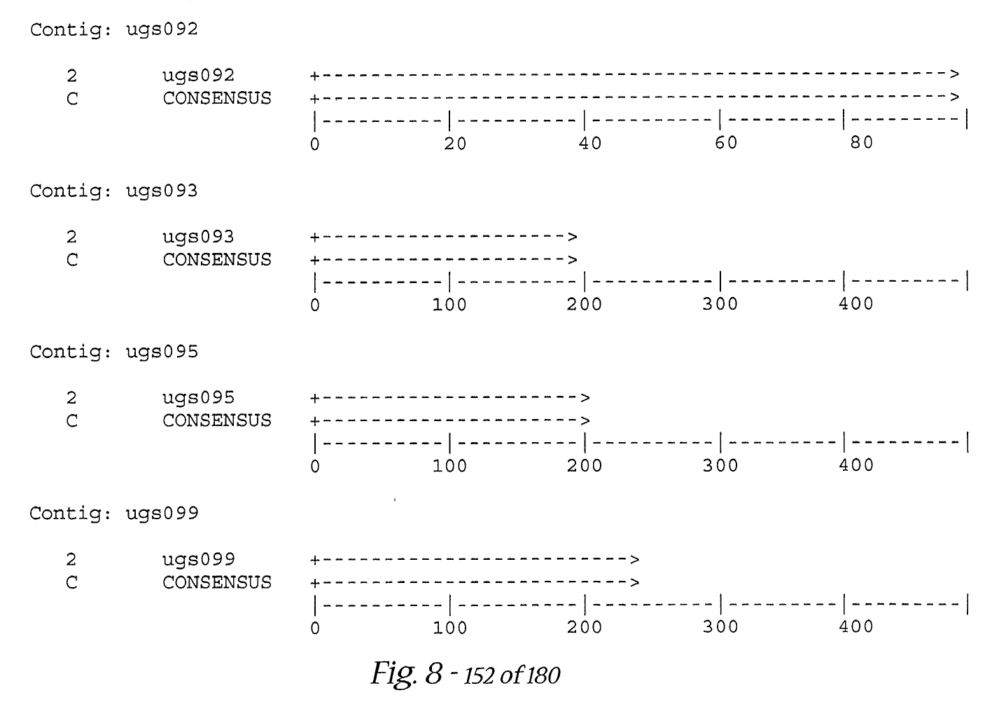

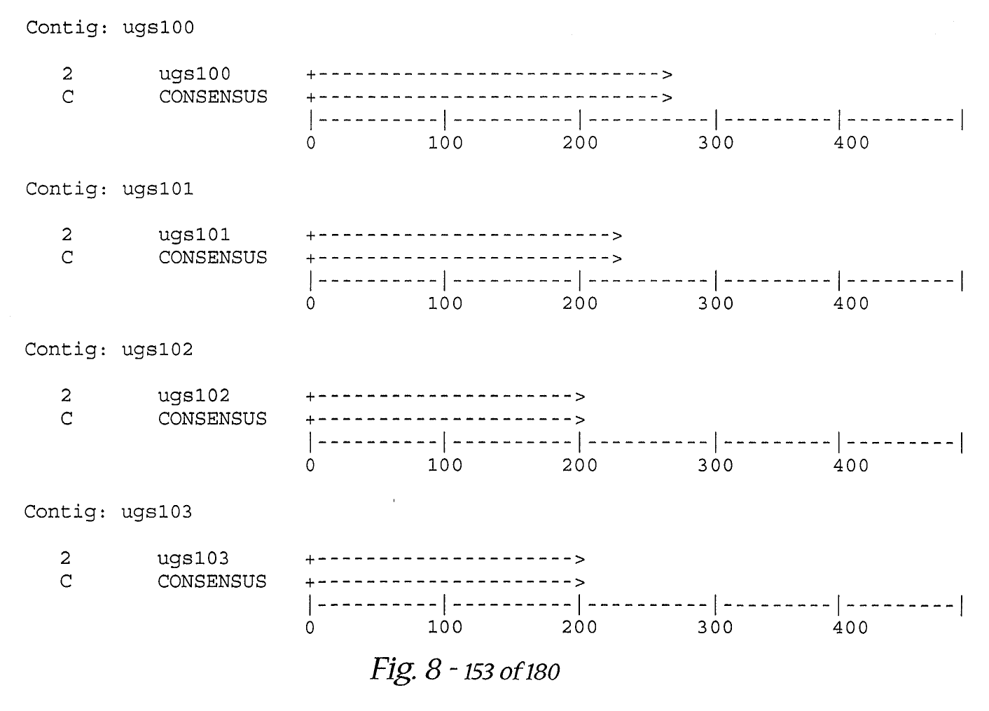

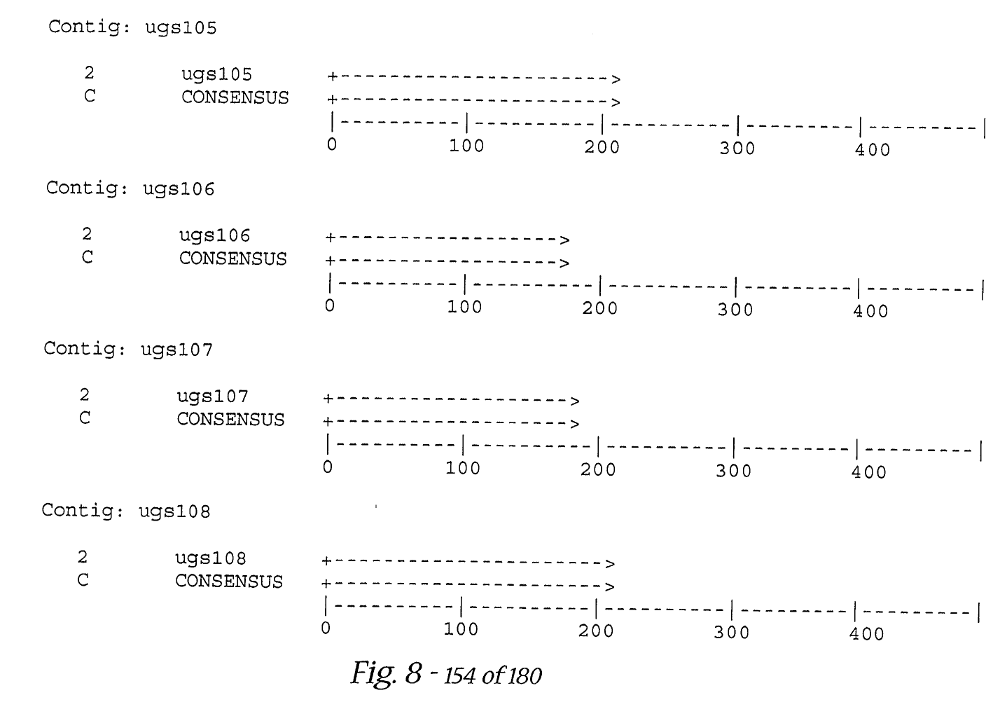

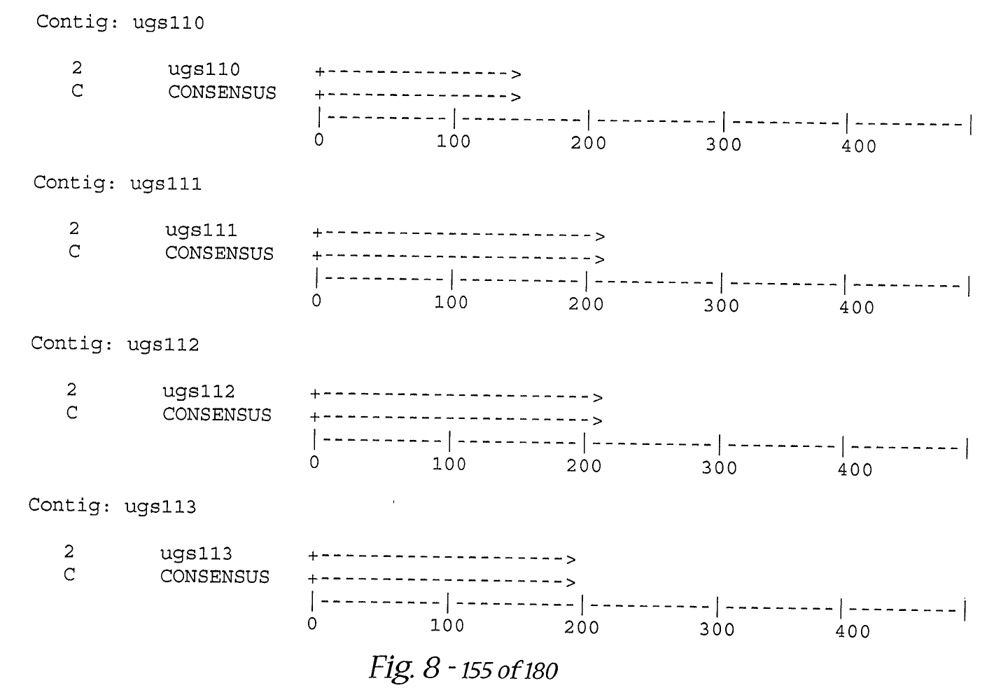

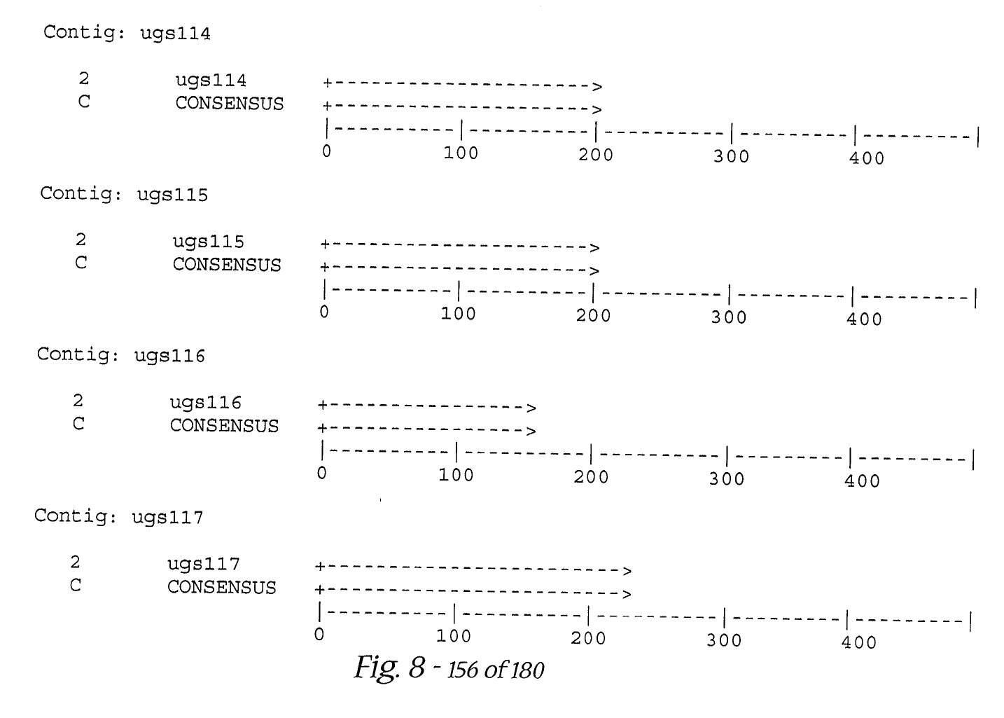

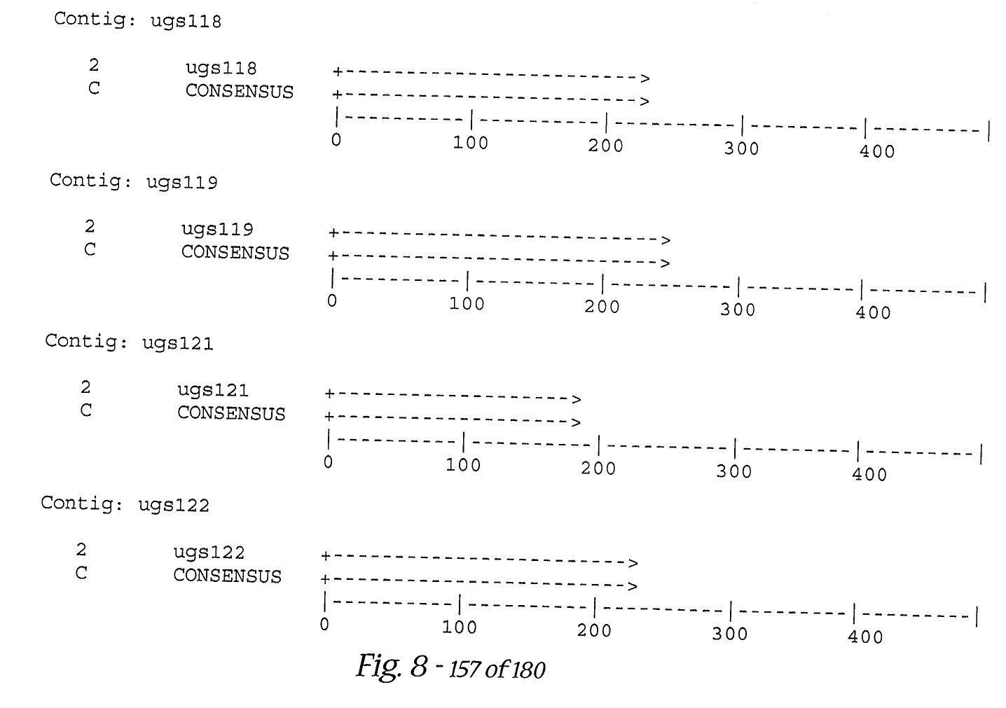

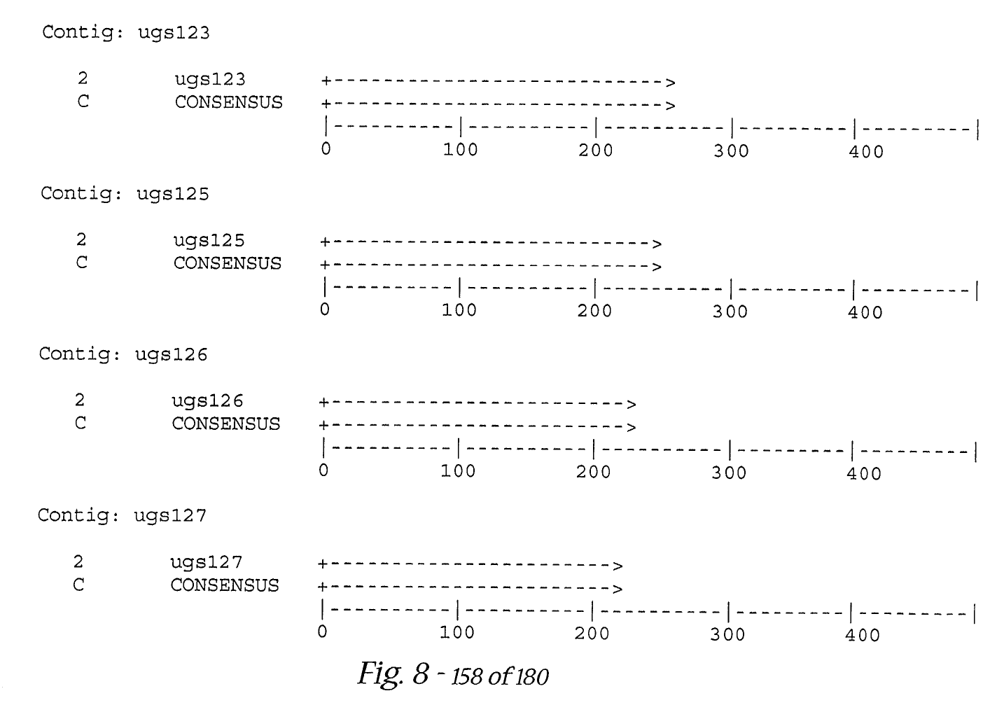

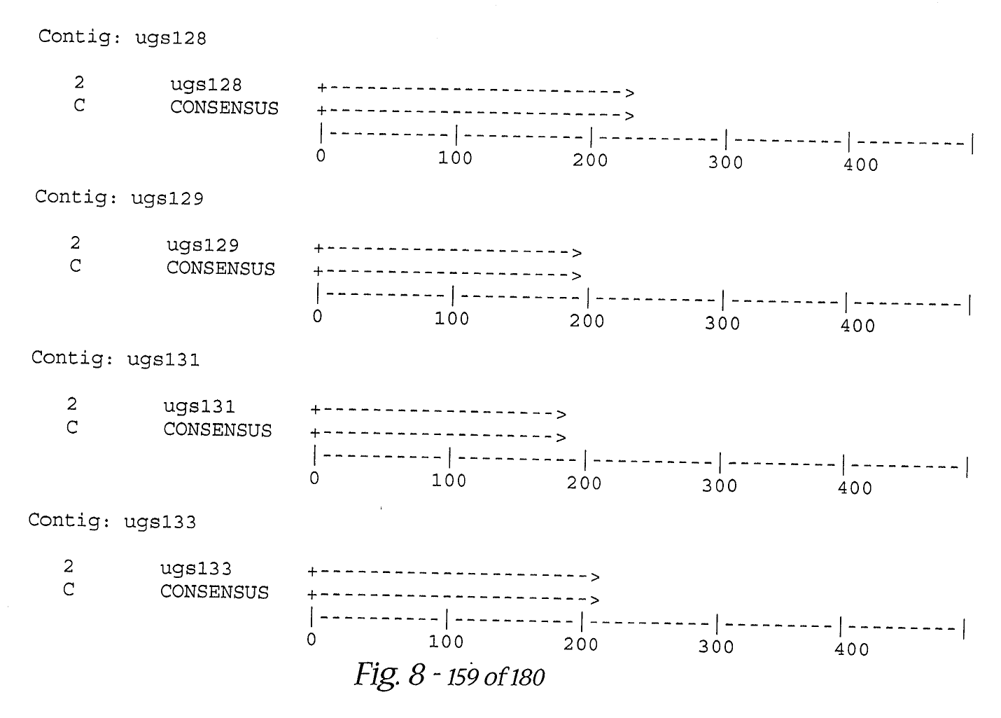

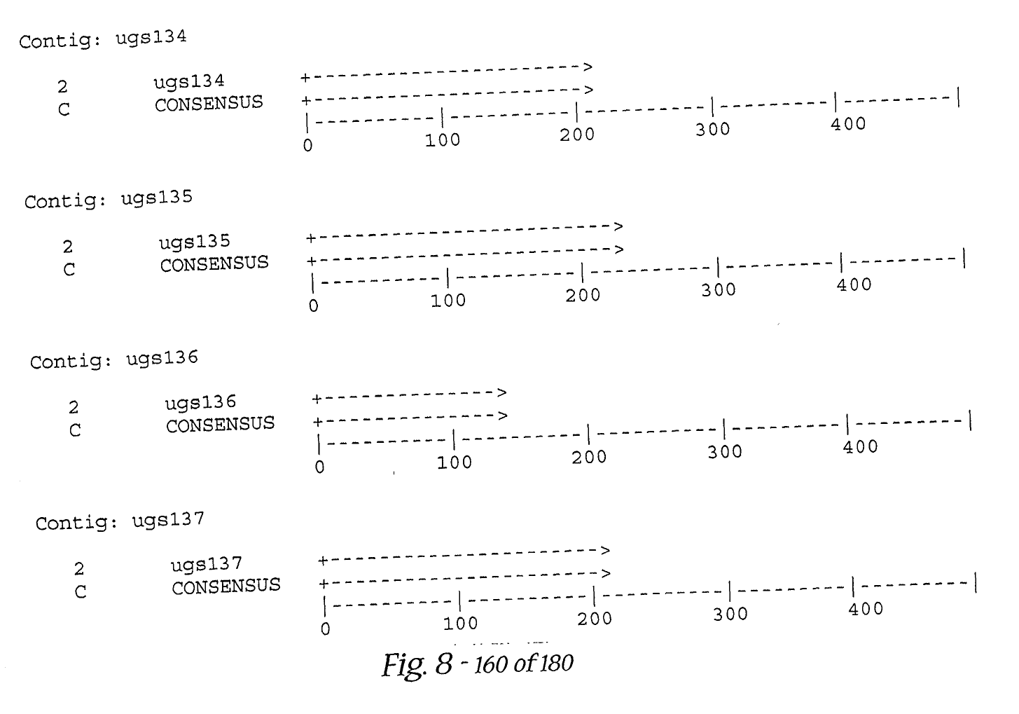

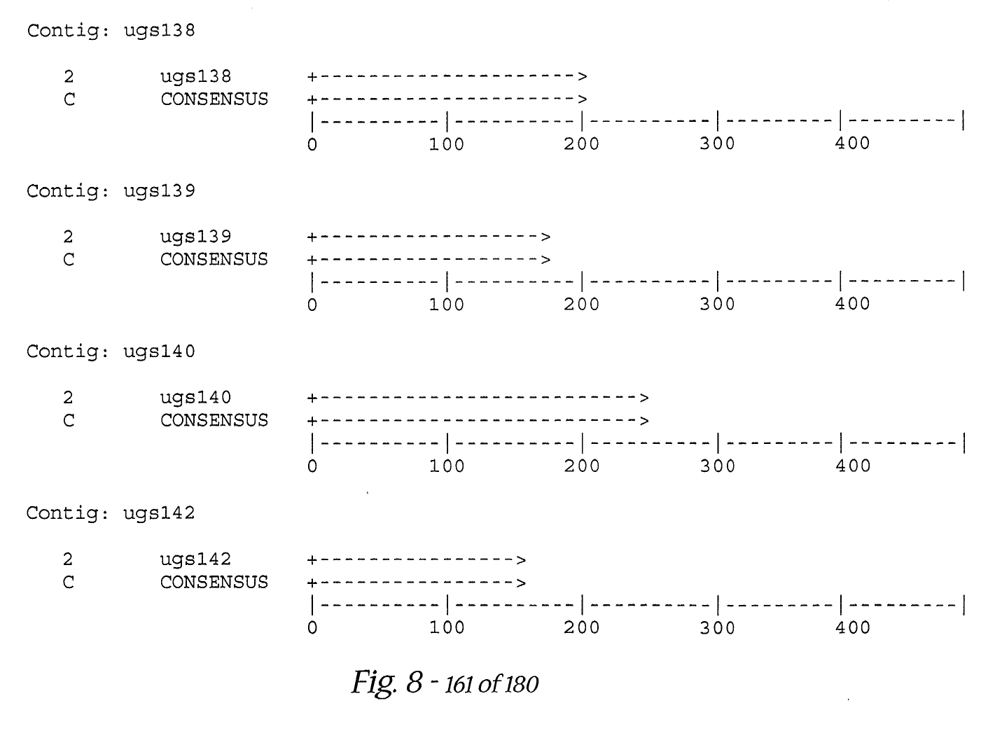

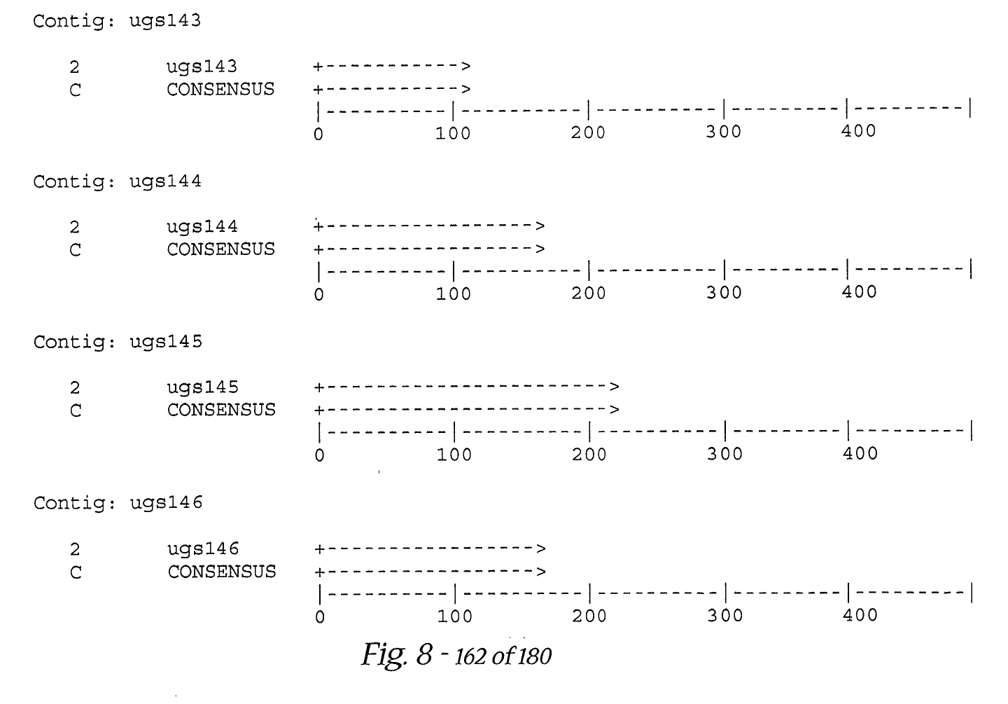

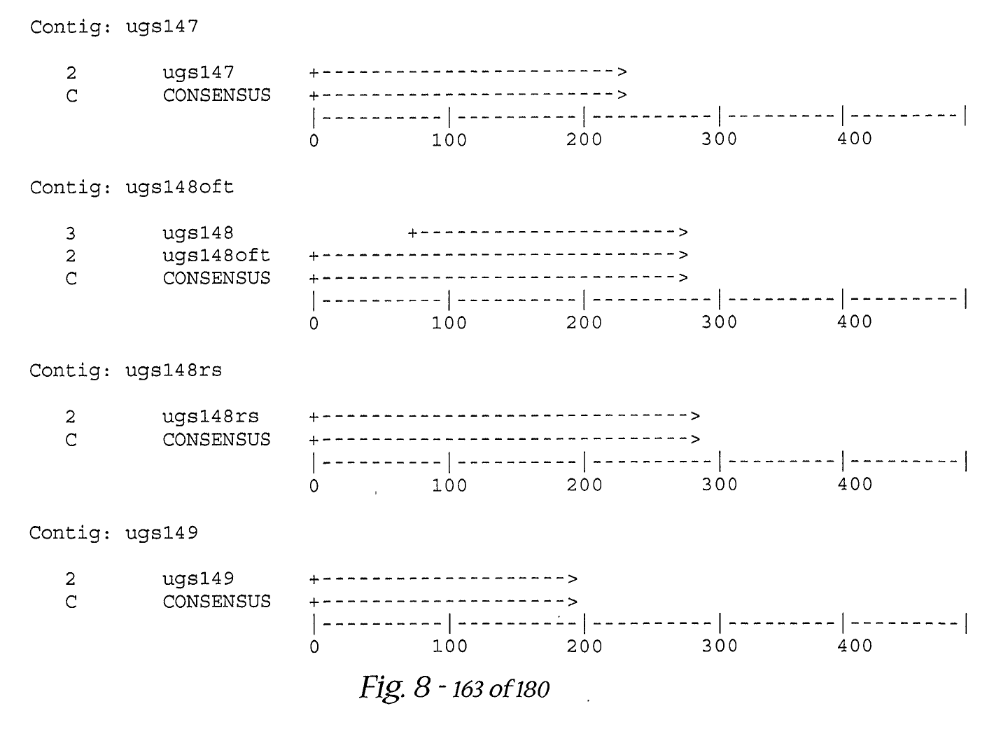

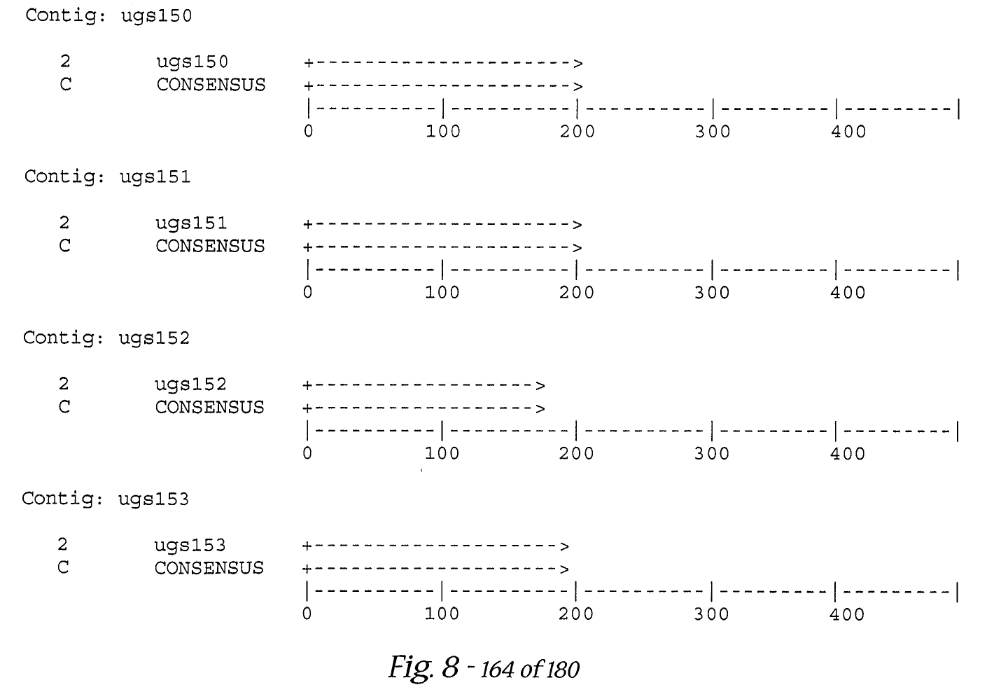

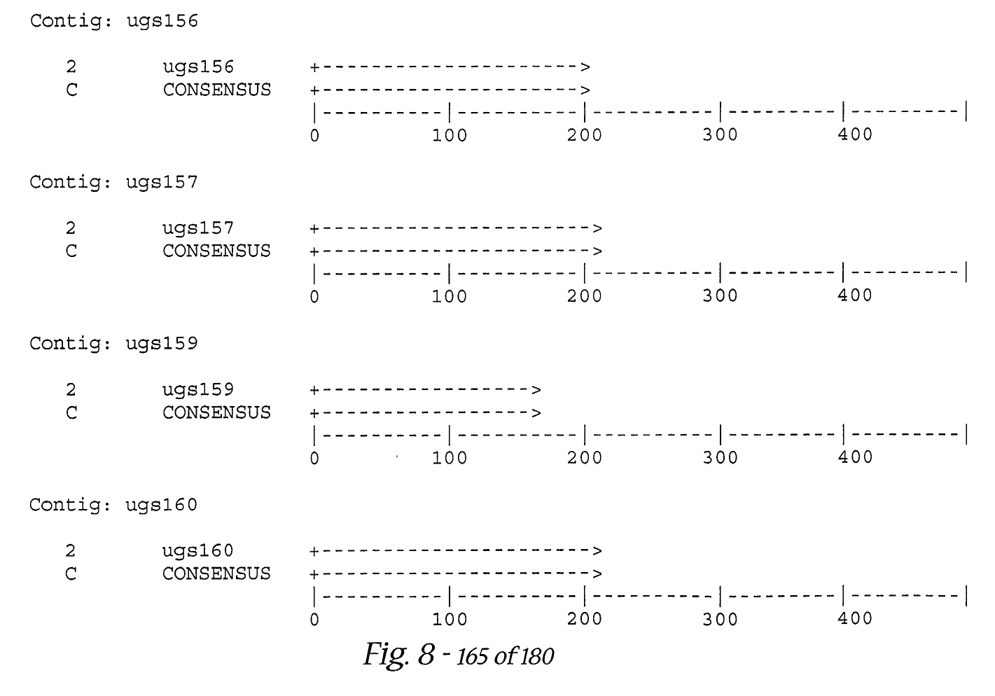

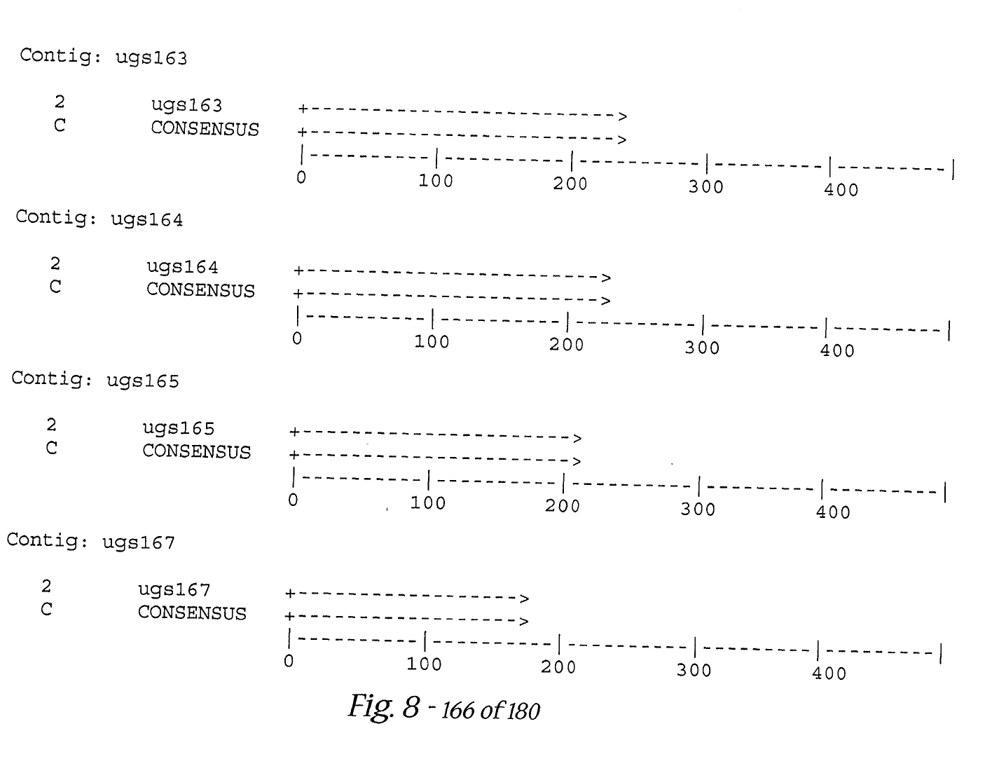

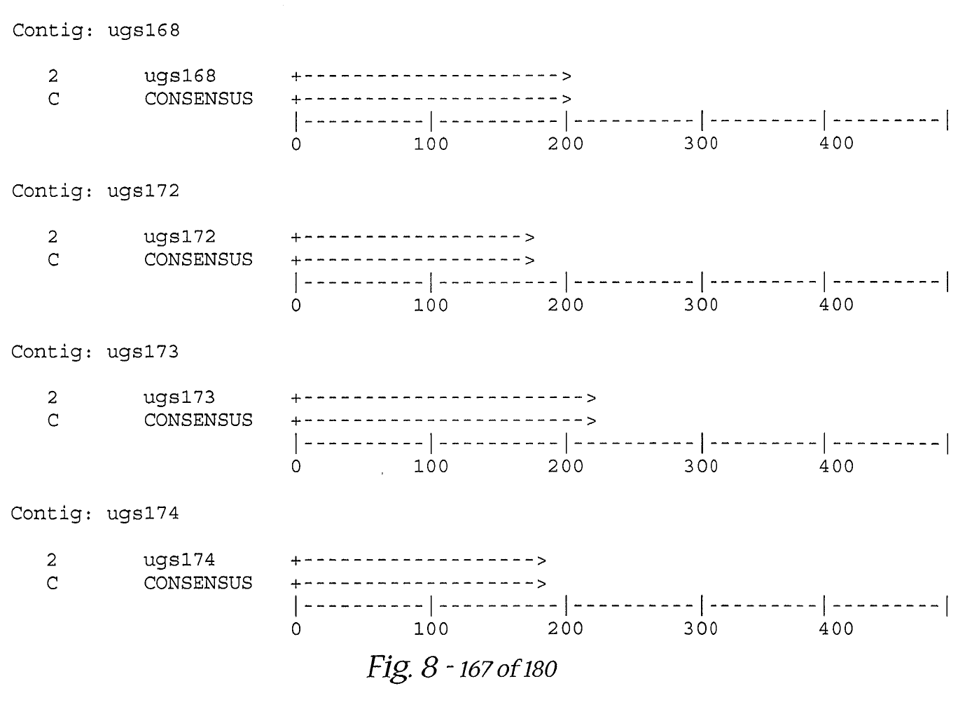

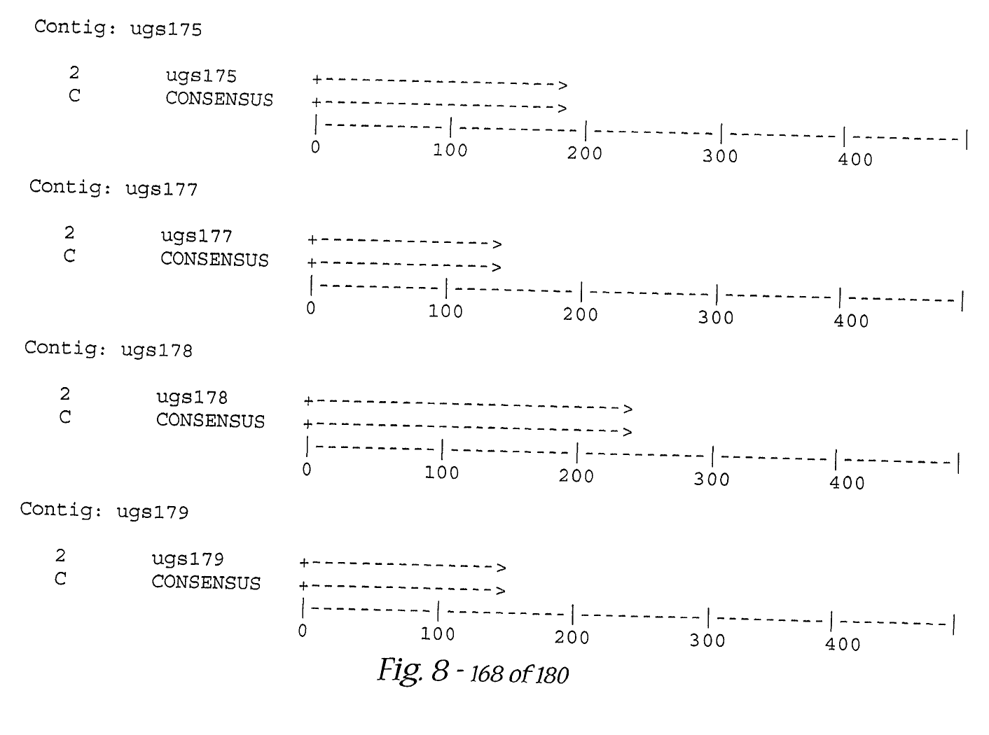

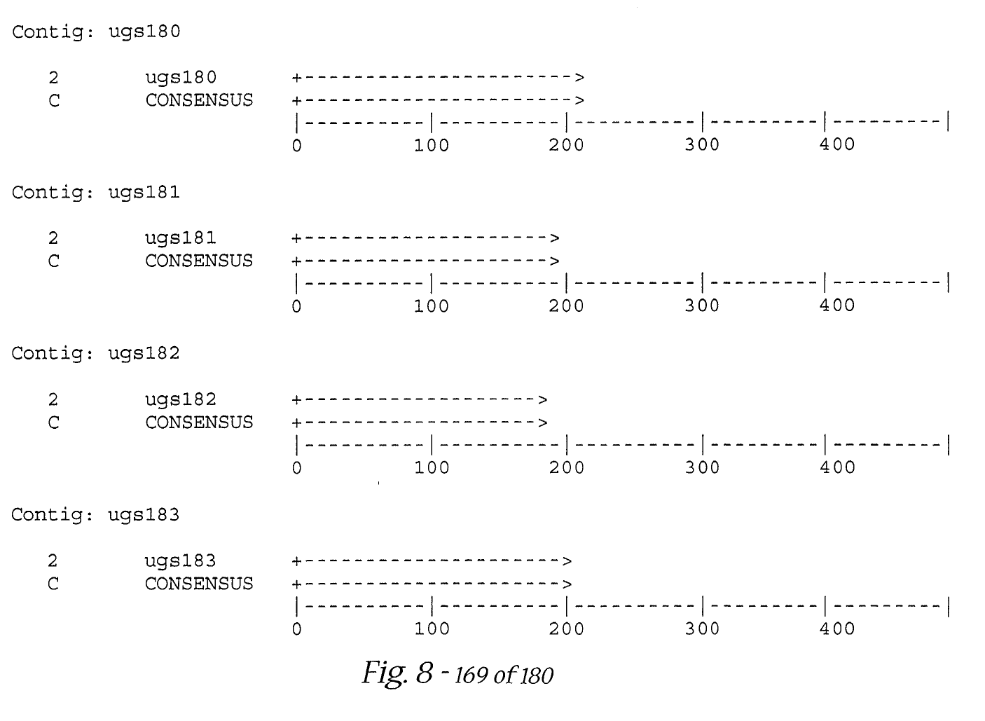

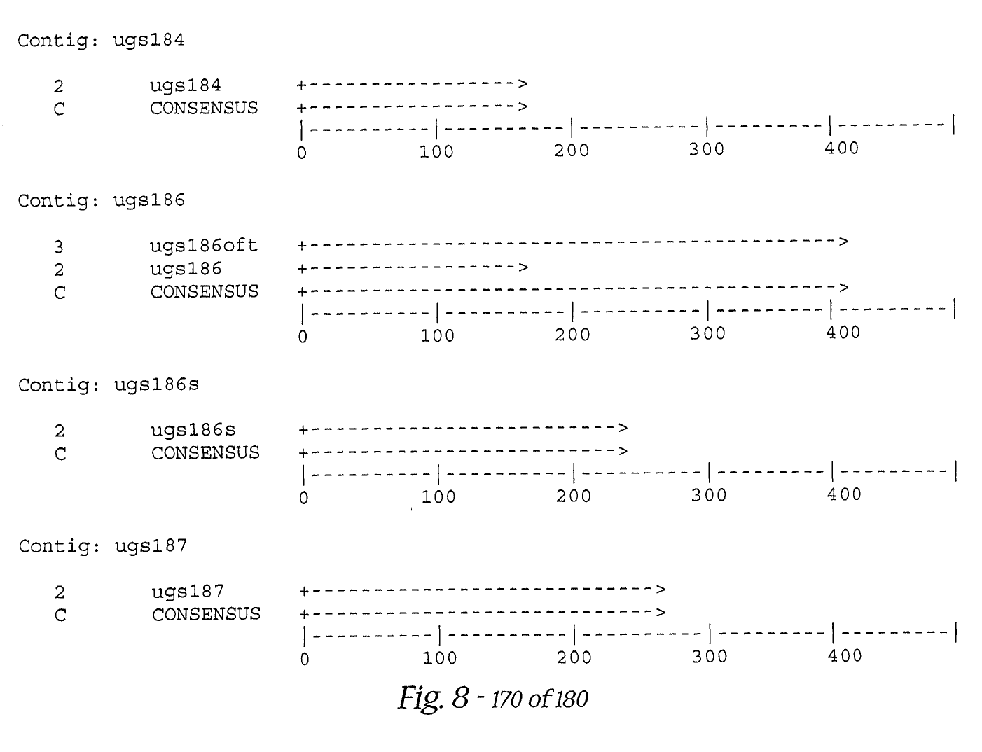

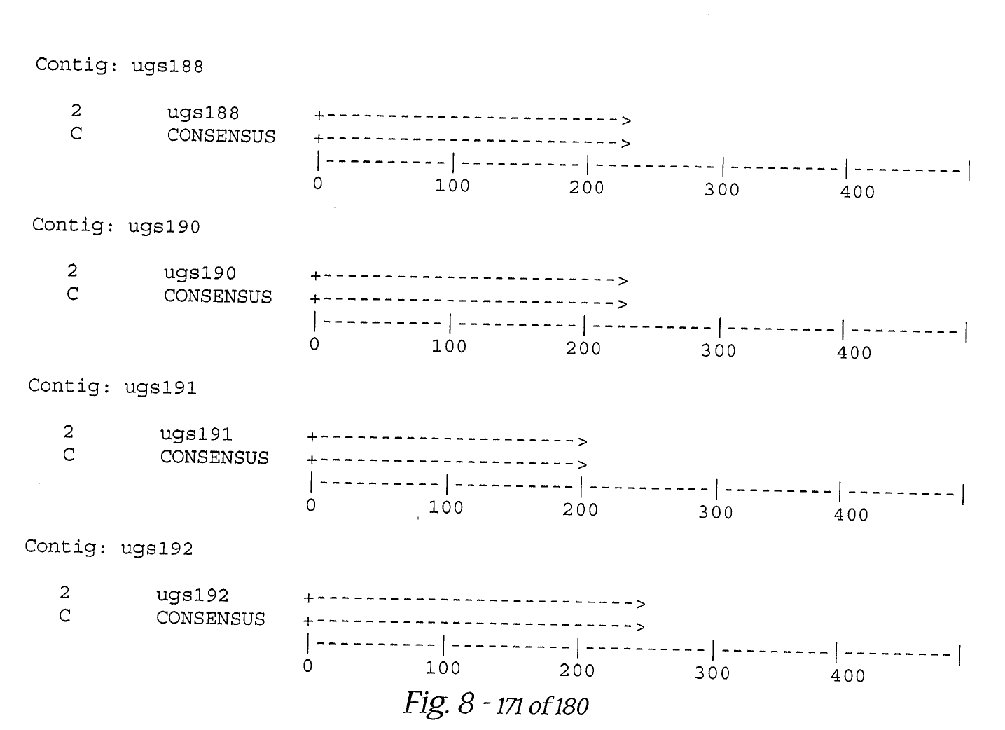

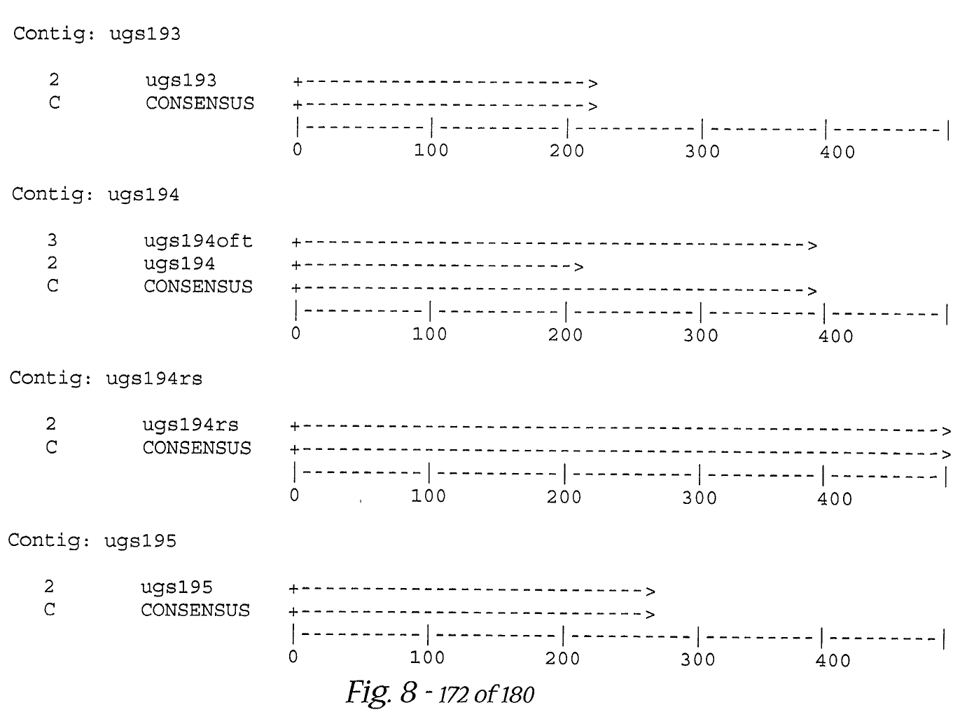

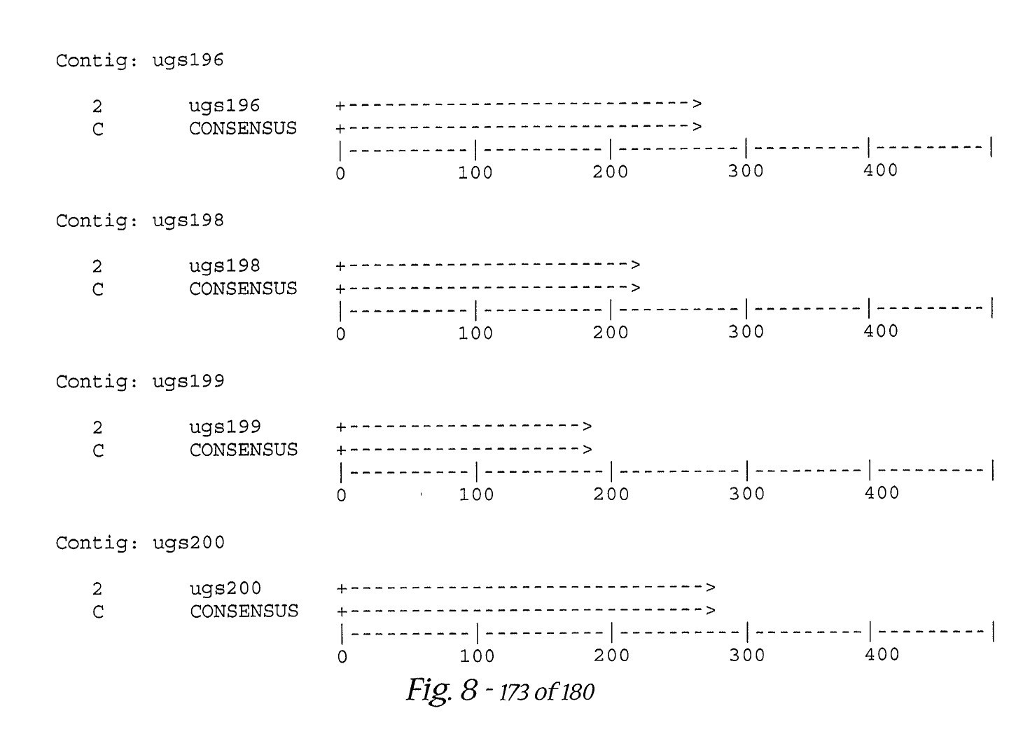

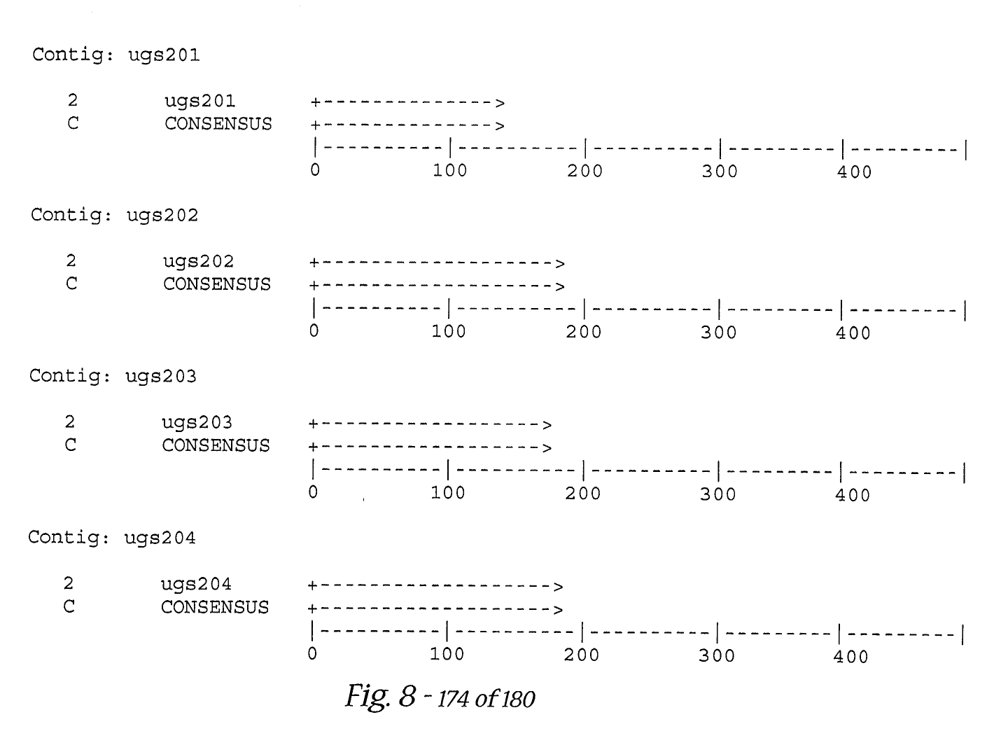

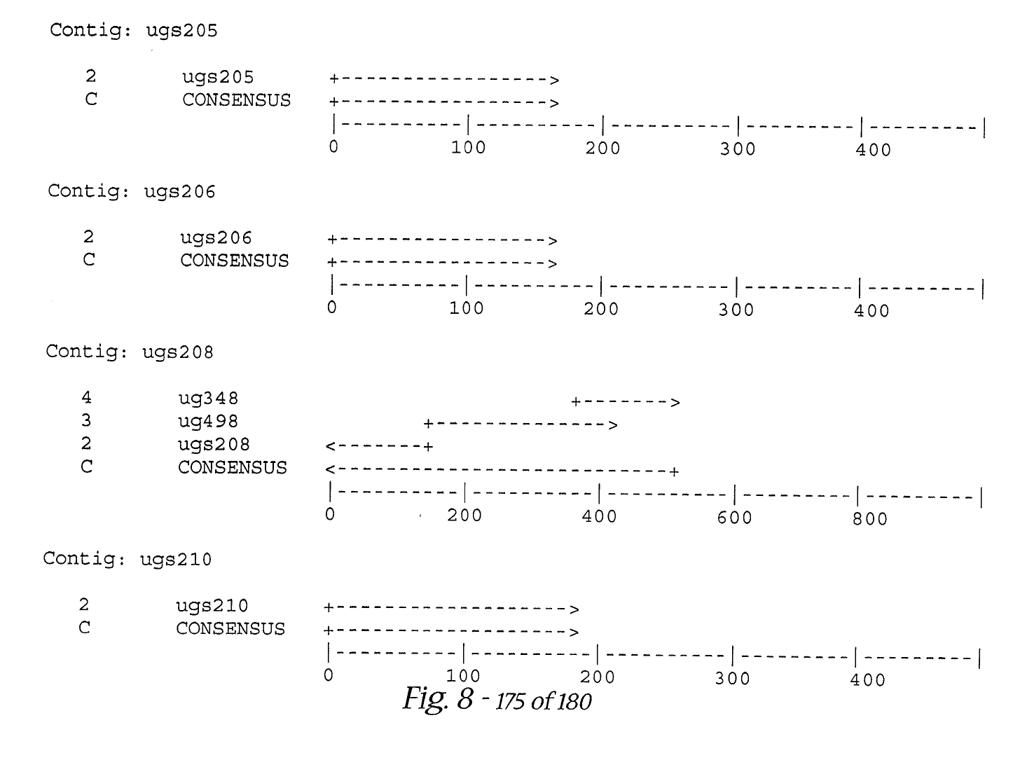

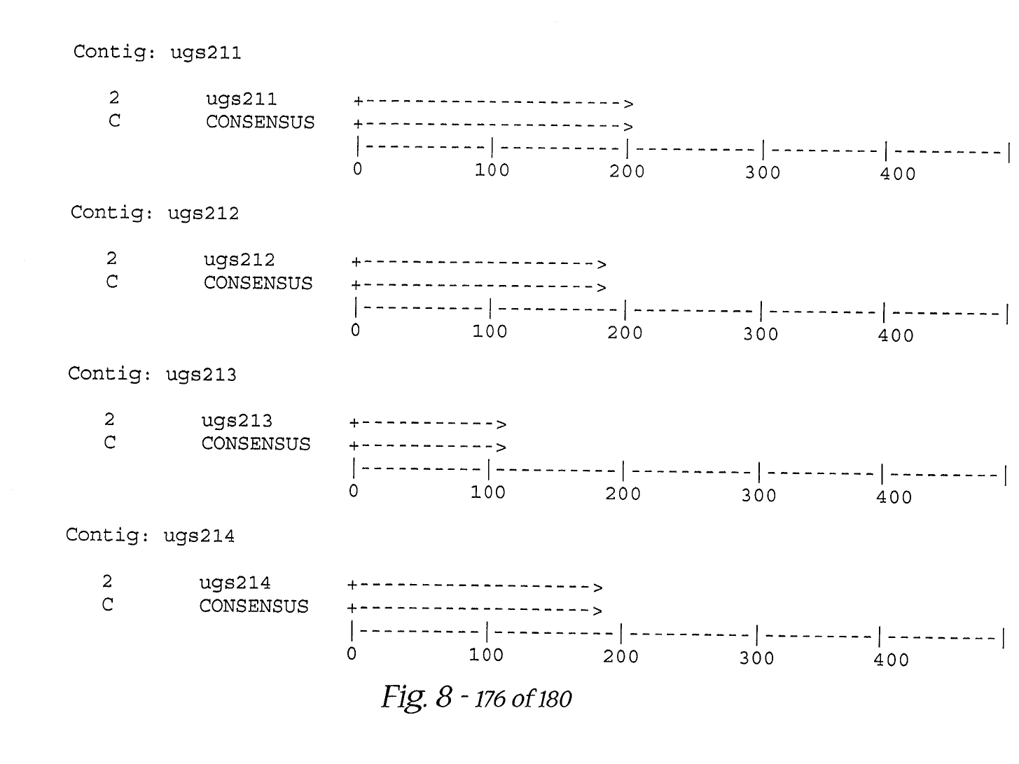

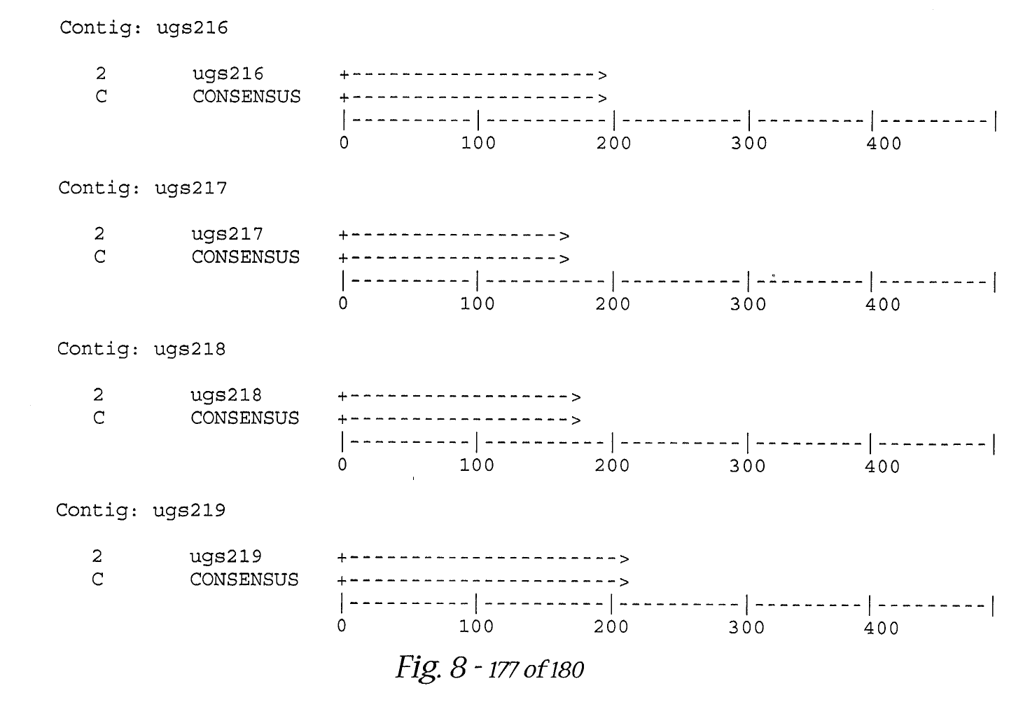

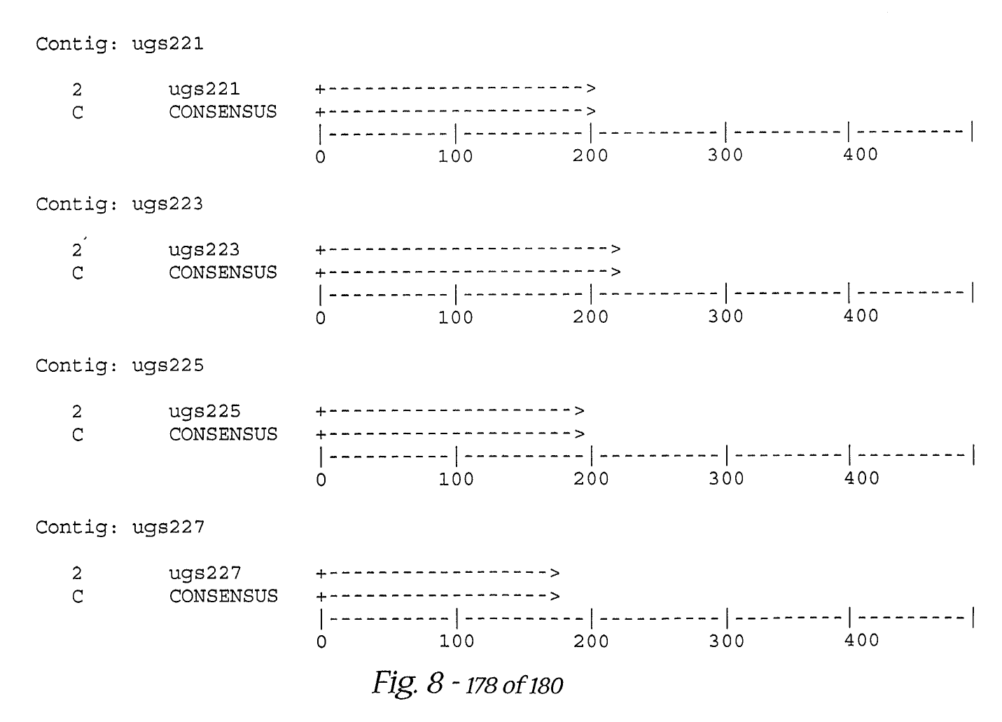

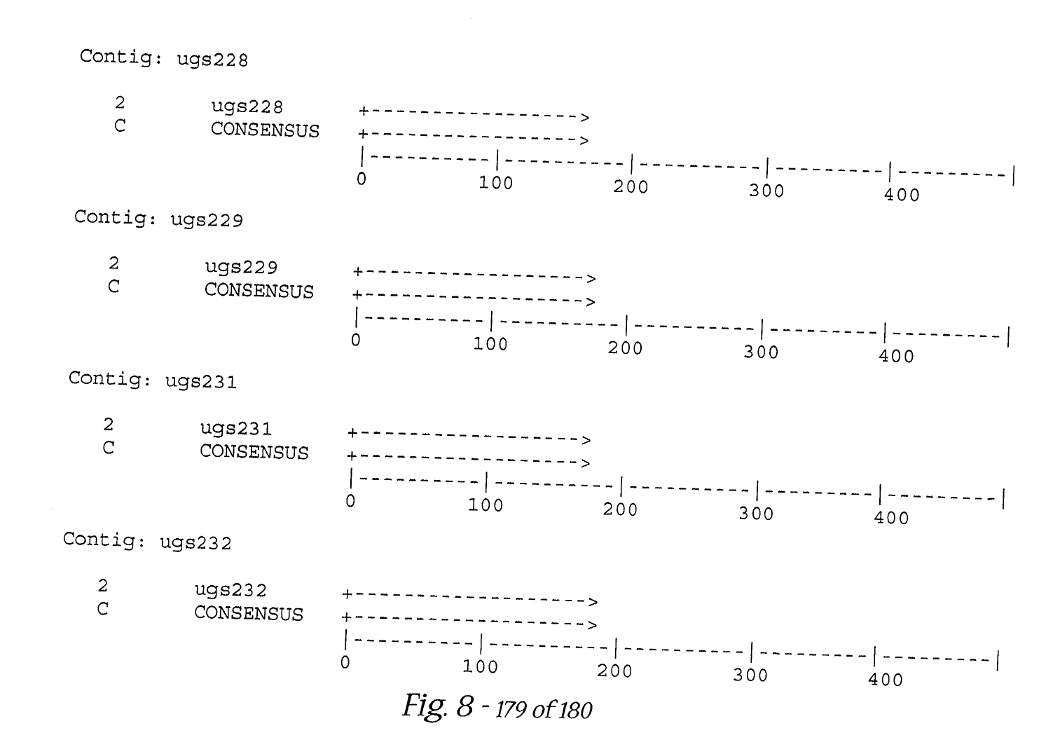

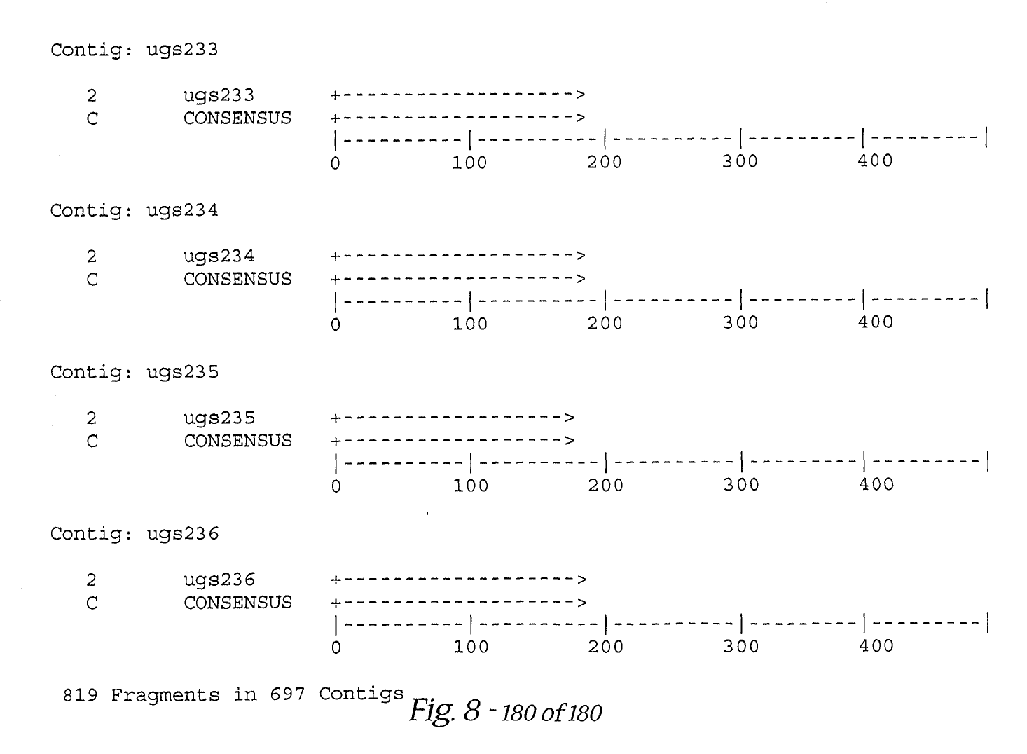

FIG. 8 represents the urogenital sinus fetal prostate cDNA clone summary obtained from GelView Contig run: A determination of the range of independent sequences. 787 cDNA clones were examined which generated 728 usable sequences as acquired. The redundancy was in the range of 2-27 times whereas the average redundancy was 2.84 times. In summary, 66 sequences. max. −44 min. sequences were represented in a contig. of 2 sequences: 33 times (11 contigs. questionable). 24 sequences max. −17 min. sequences were represented in a contig of 3 sequences: 8 times (7 seq. questionable). 5 sequences were represented in a contig. of 5 sequences: 1 time (none questionable). 27 seq. were represented in a contig. of 27 sequences: 1 time (none questionable). Therefore, this result represents 43 generated contig events representing 122 sequences max. and 93 sequences min. in overlapping contigs. Thus, the max. number of single representation is: 728−93=635 single clones+43 seq. contigs.=678 individual sequences. Thus, the min. number of single representation is: 728−122=606 single clones+43 sequences contigs.=649 individual sequences.

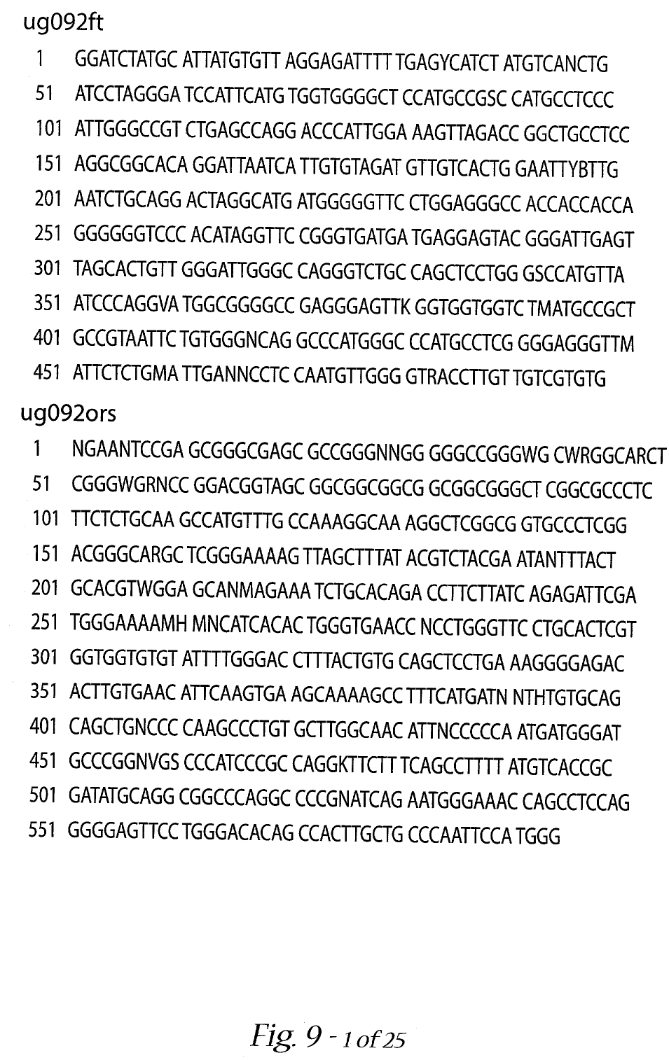

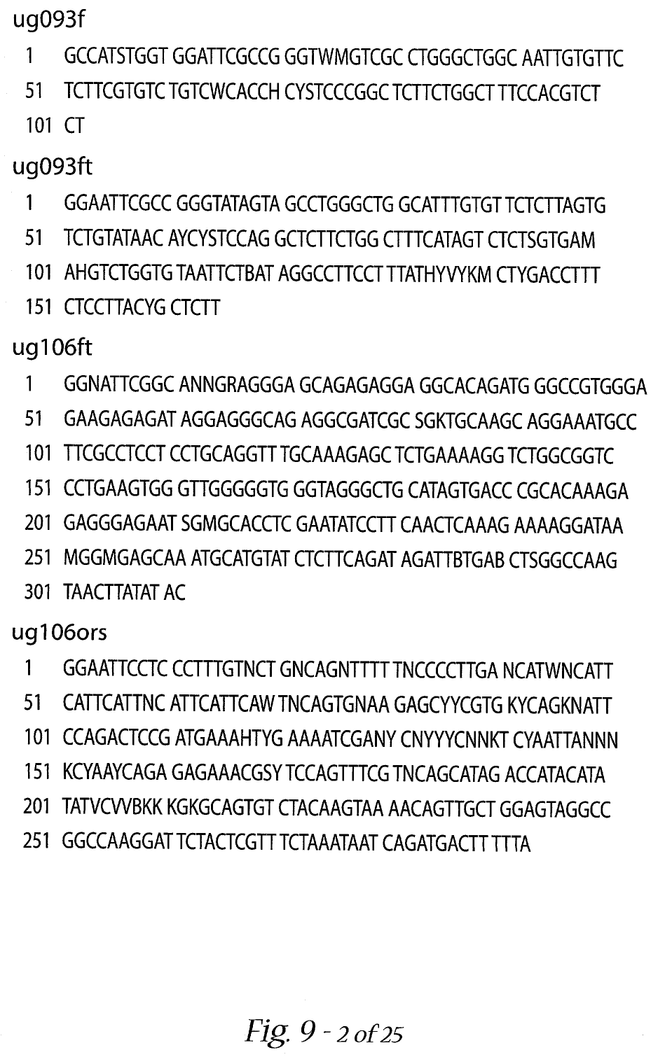

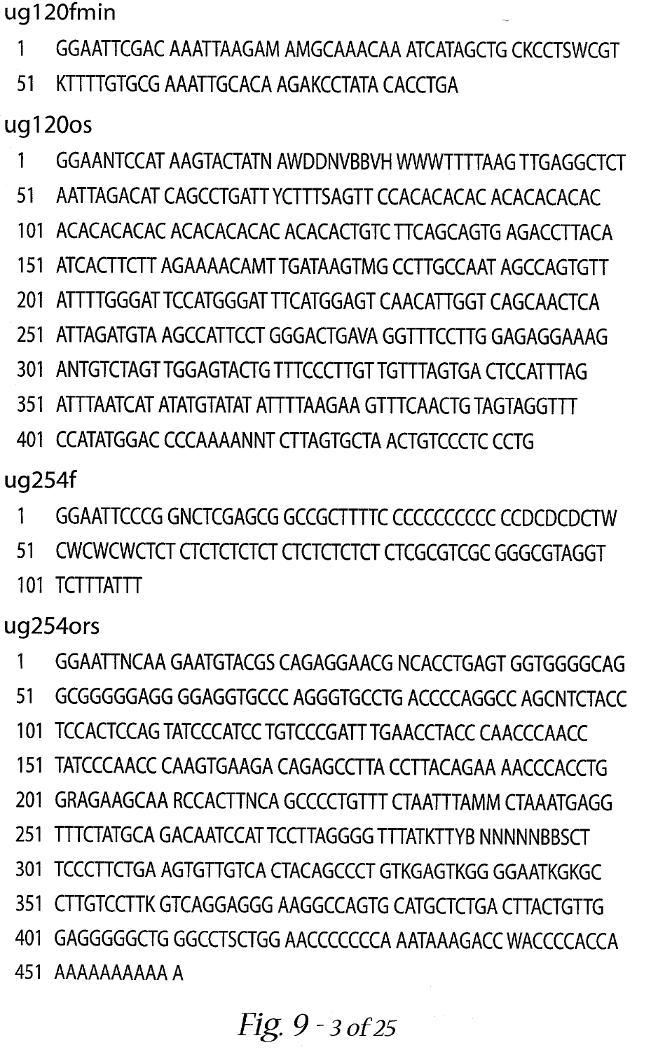

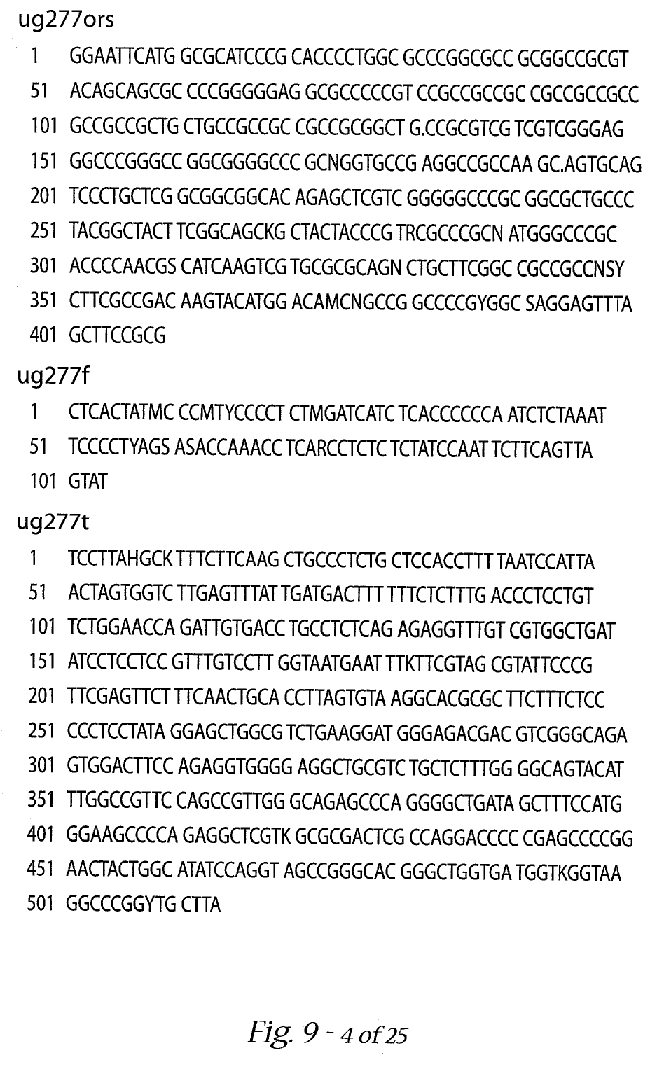

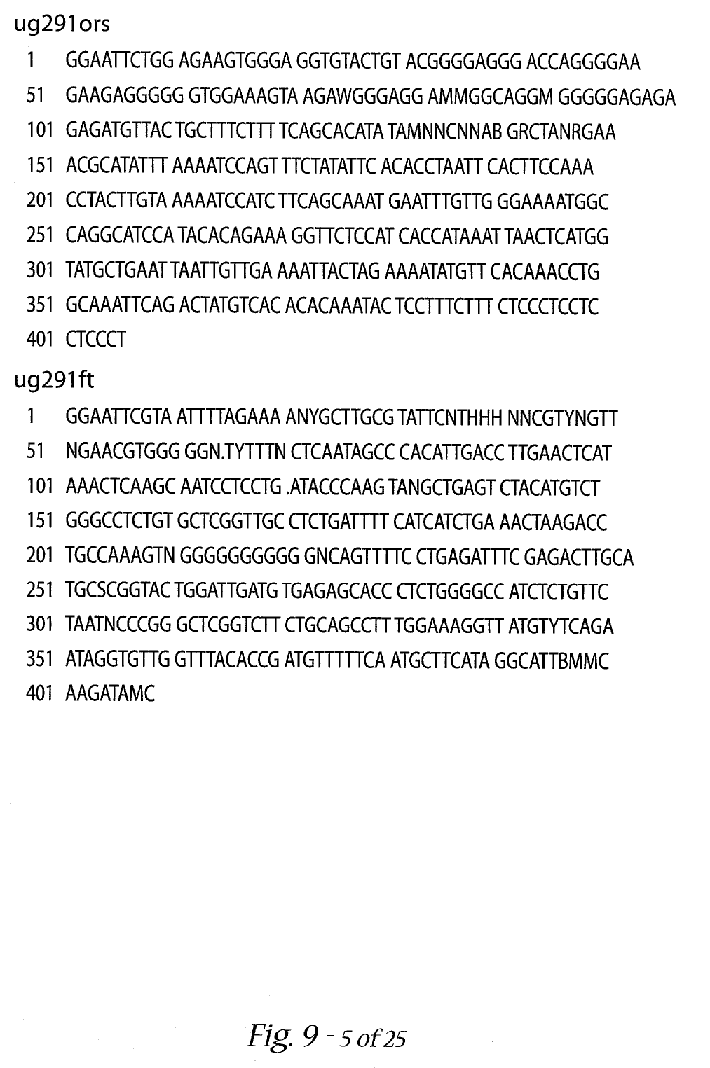

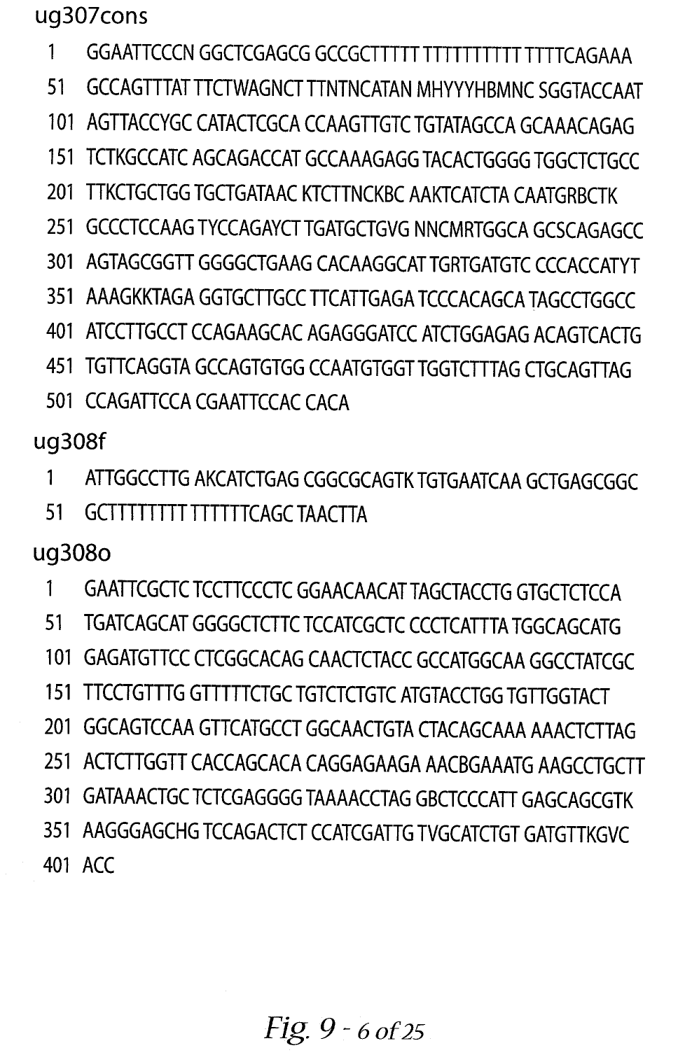

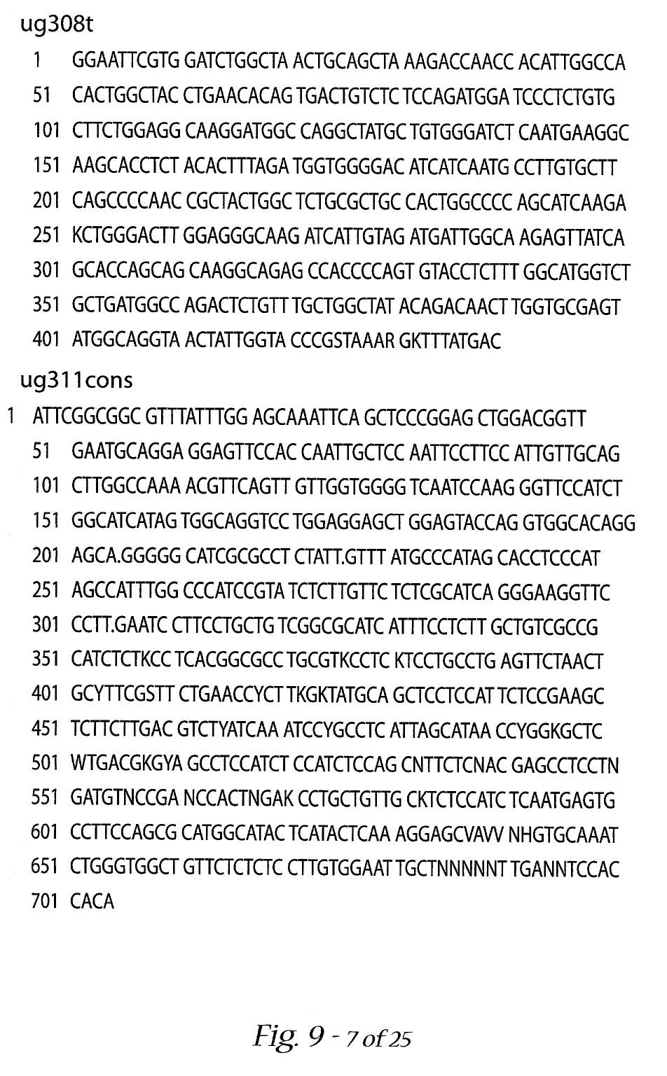

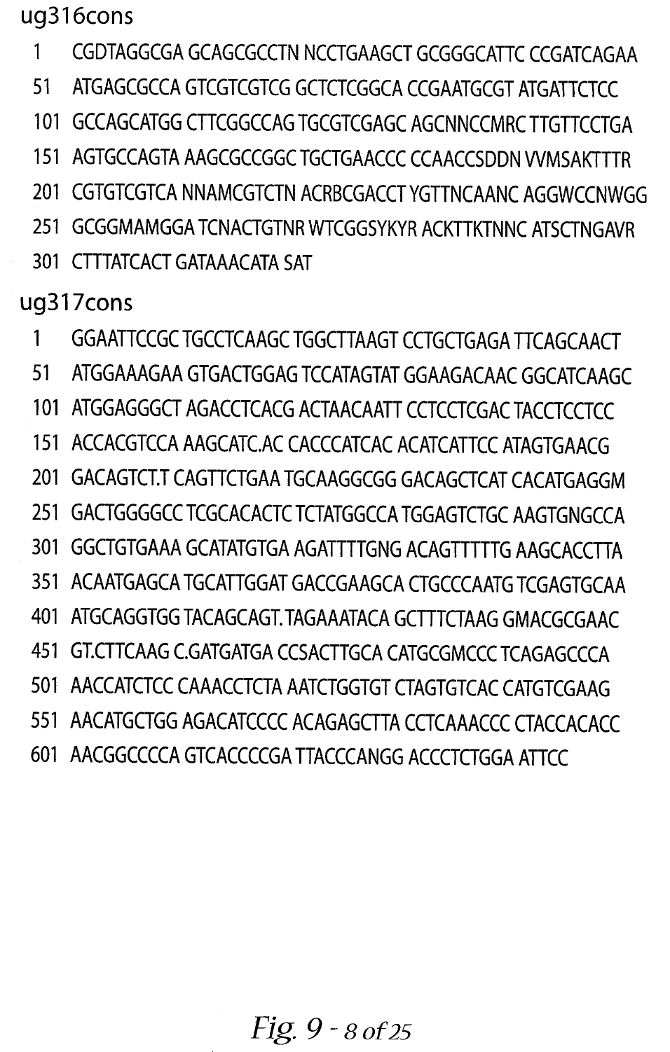

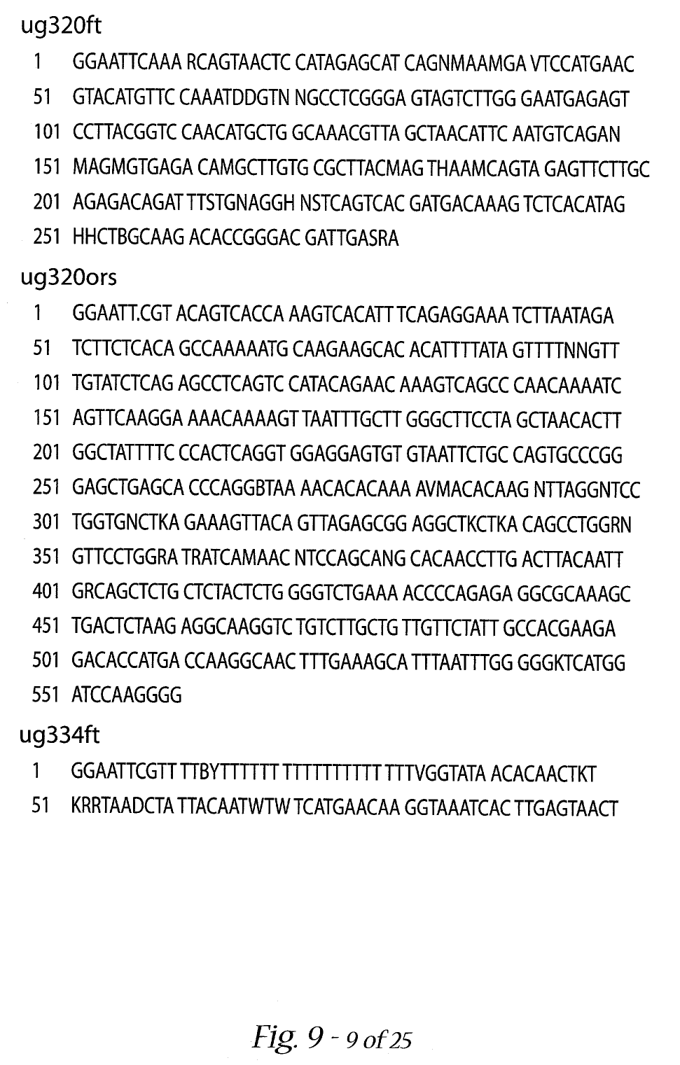

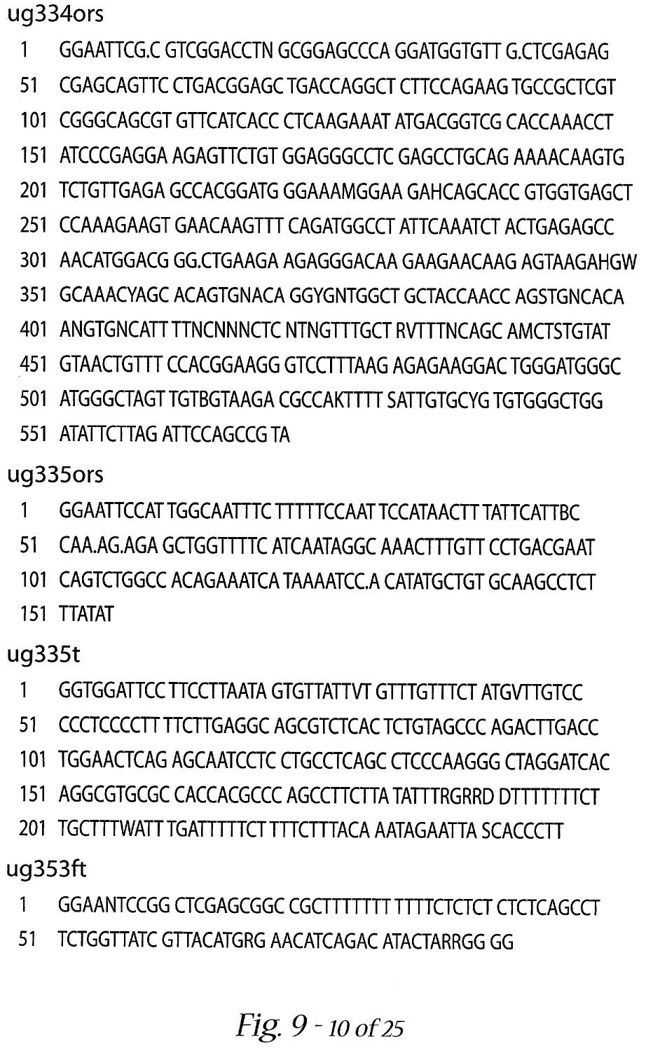

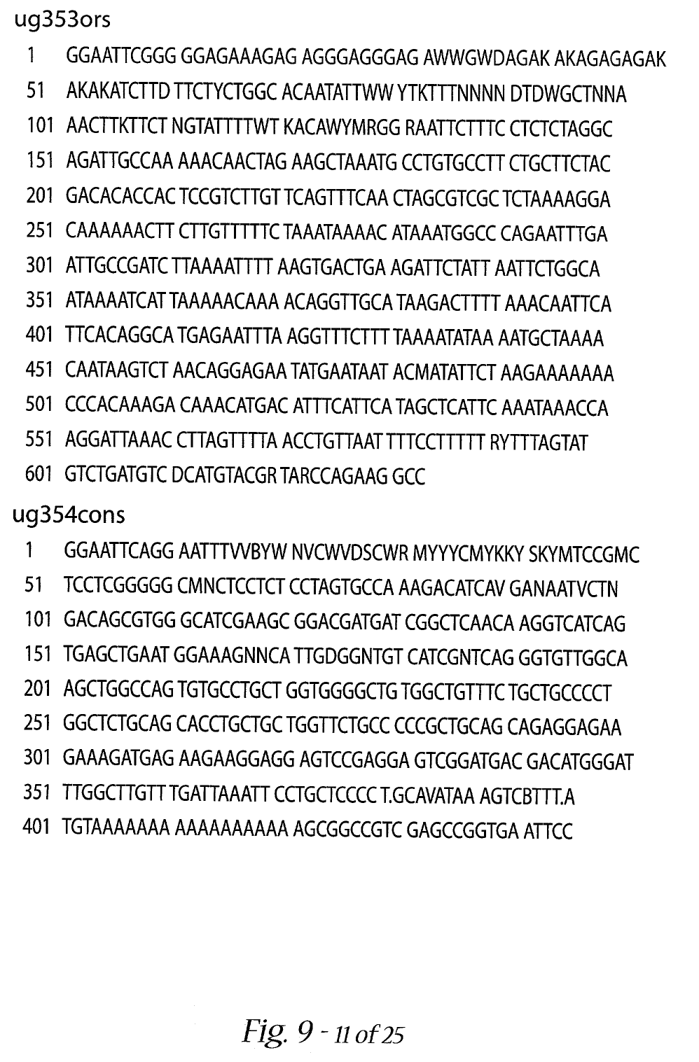

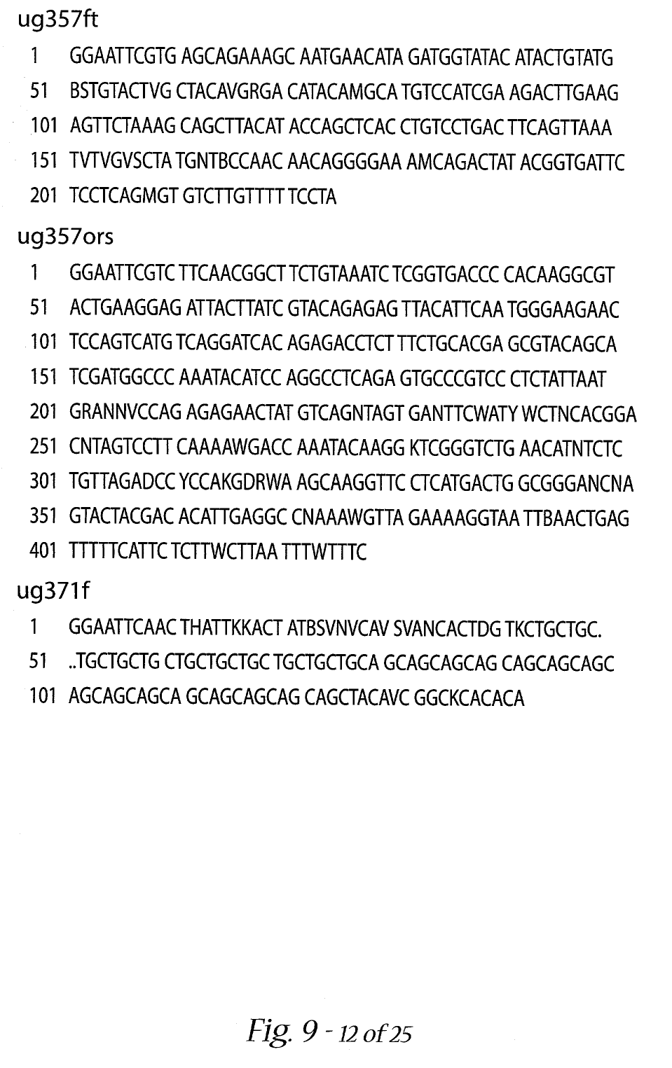

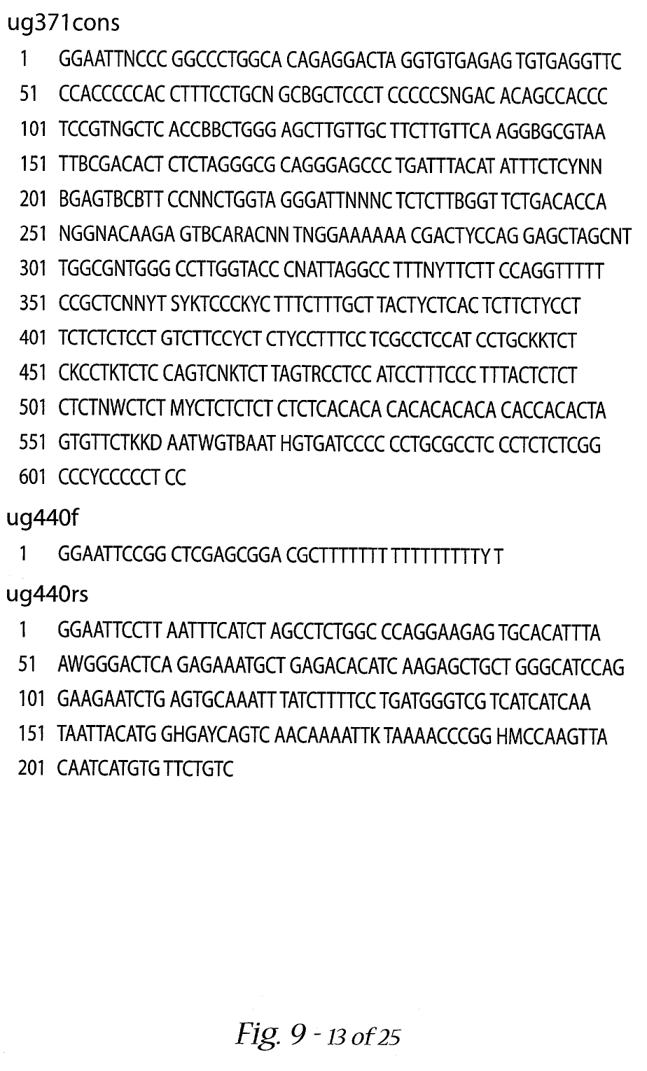

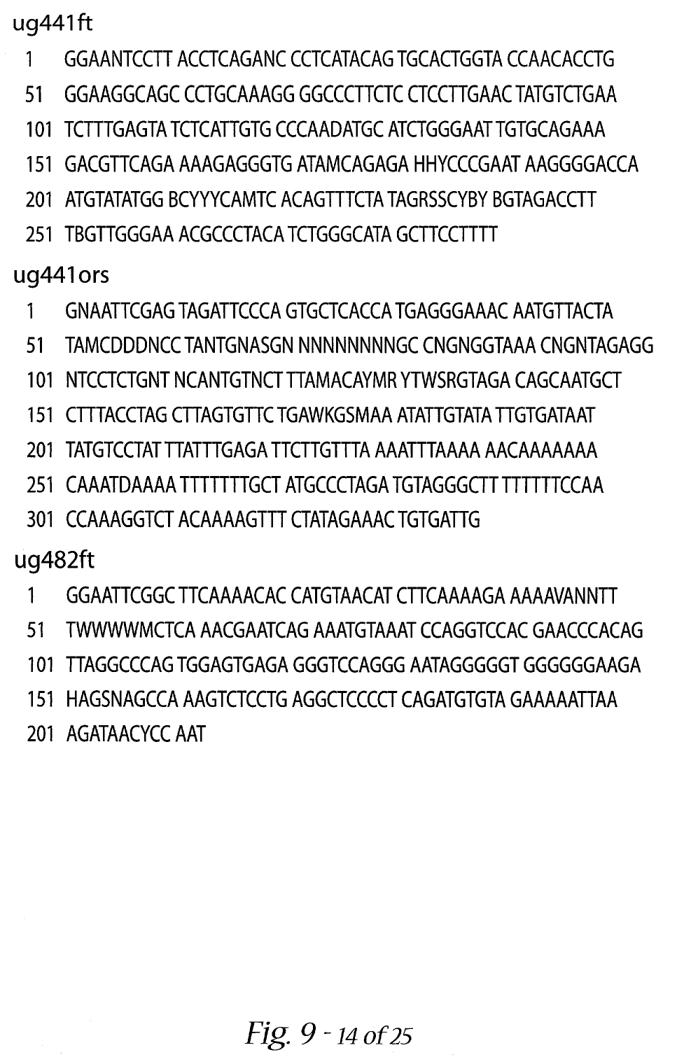

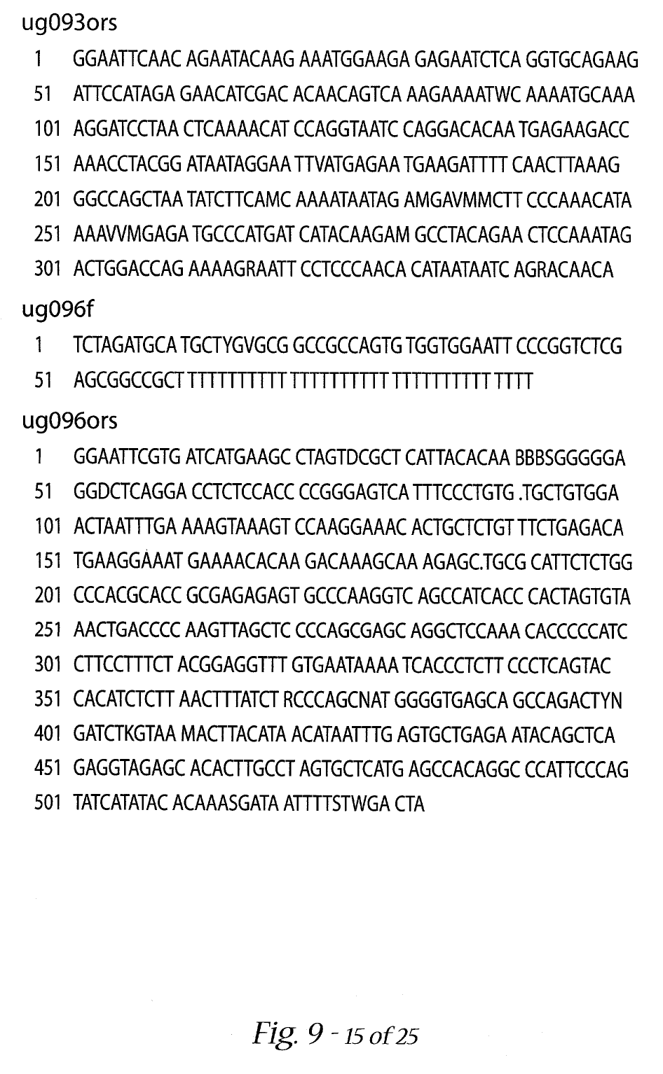

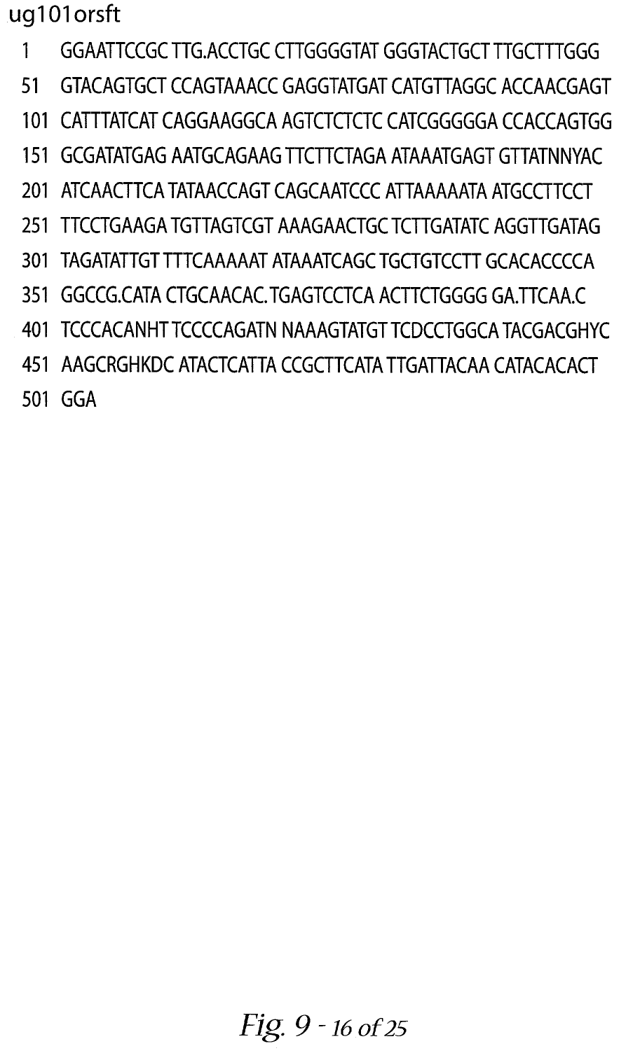

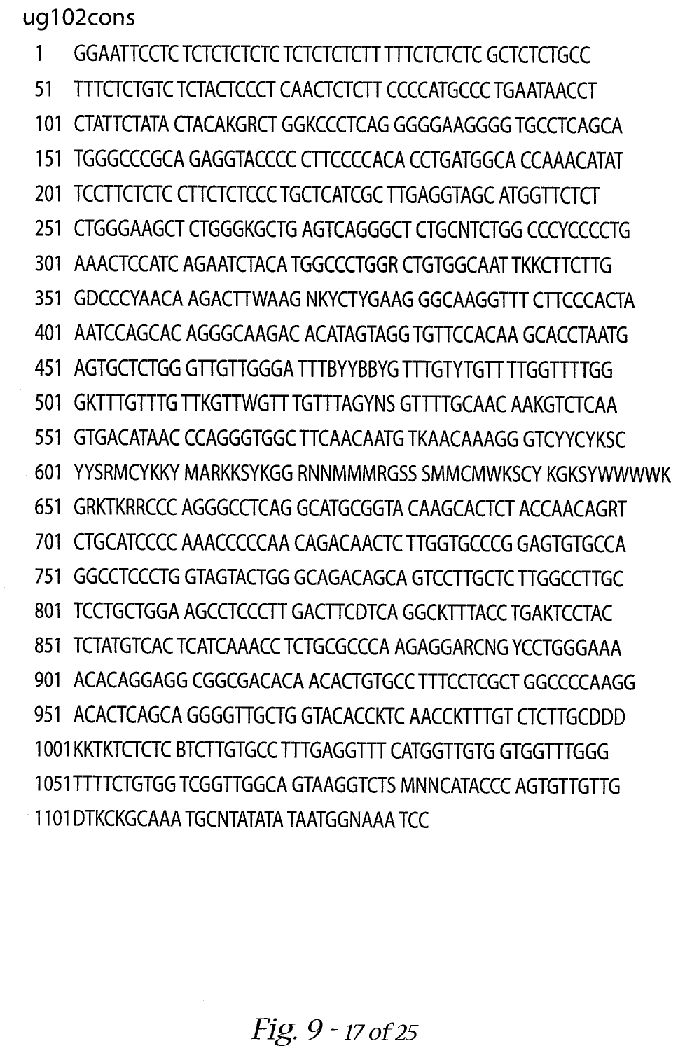

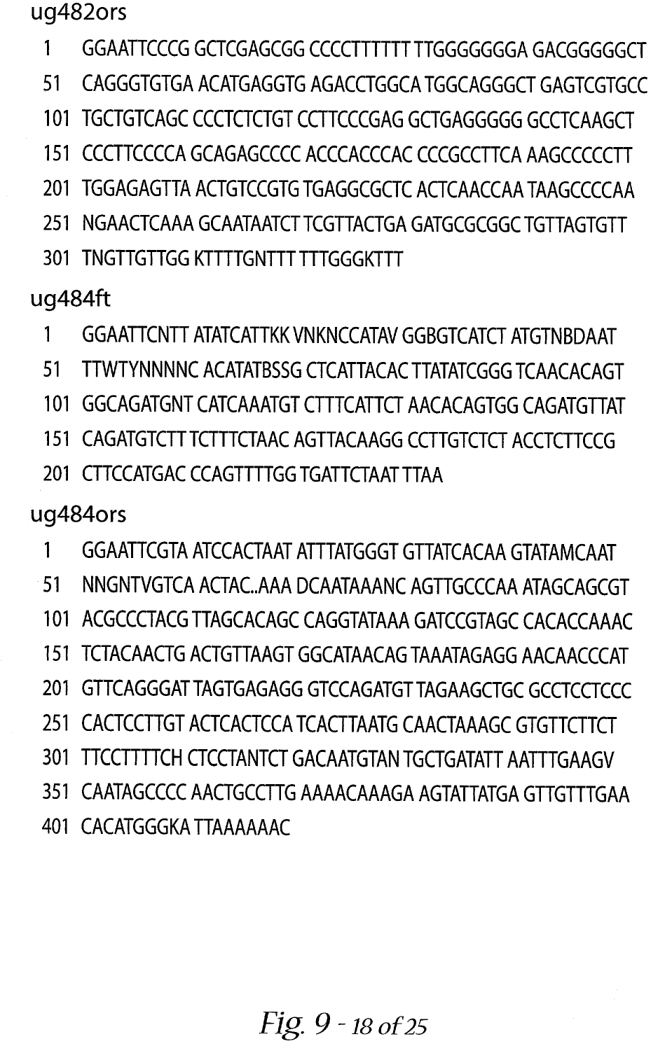

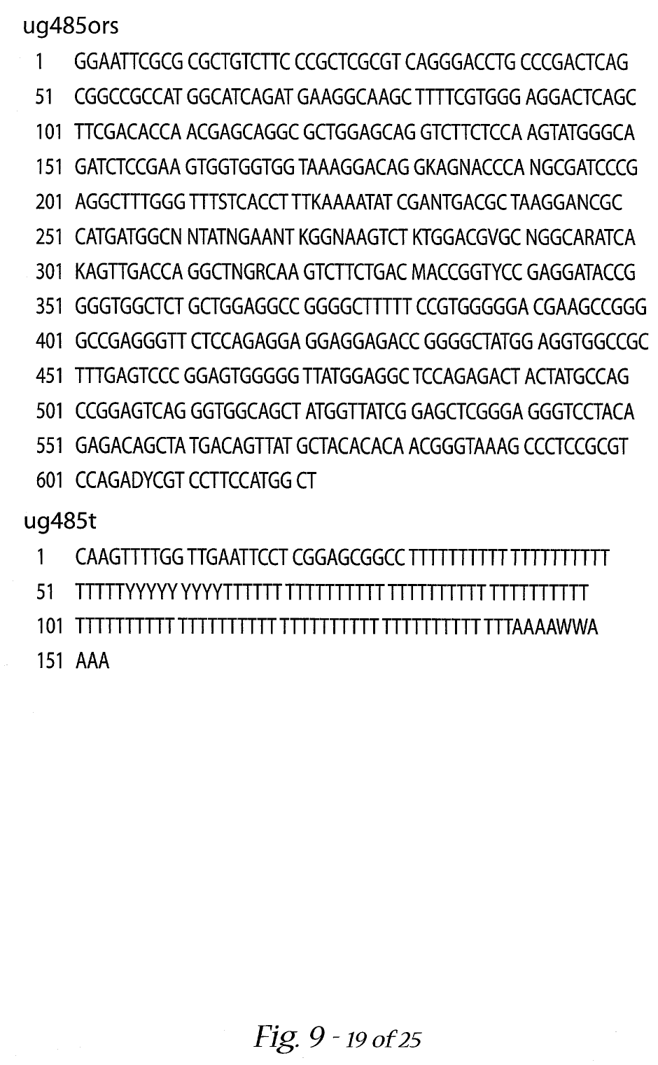

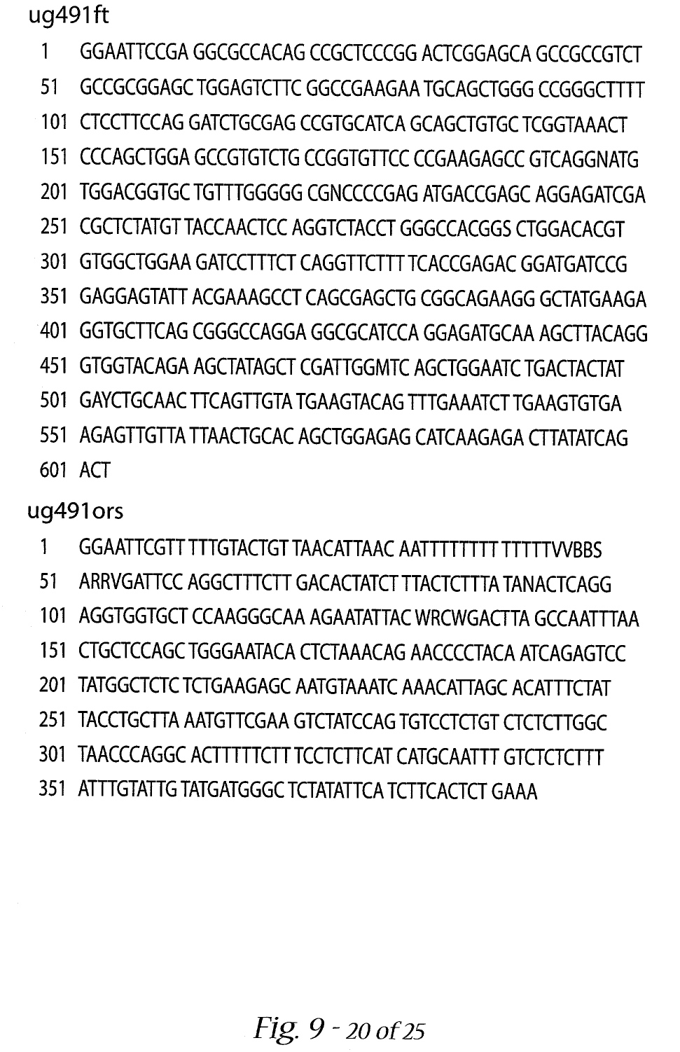

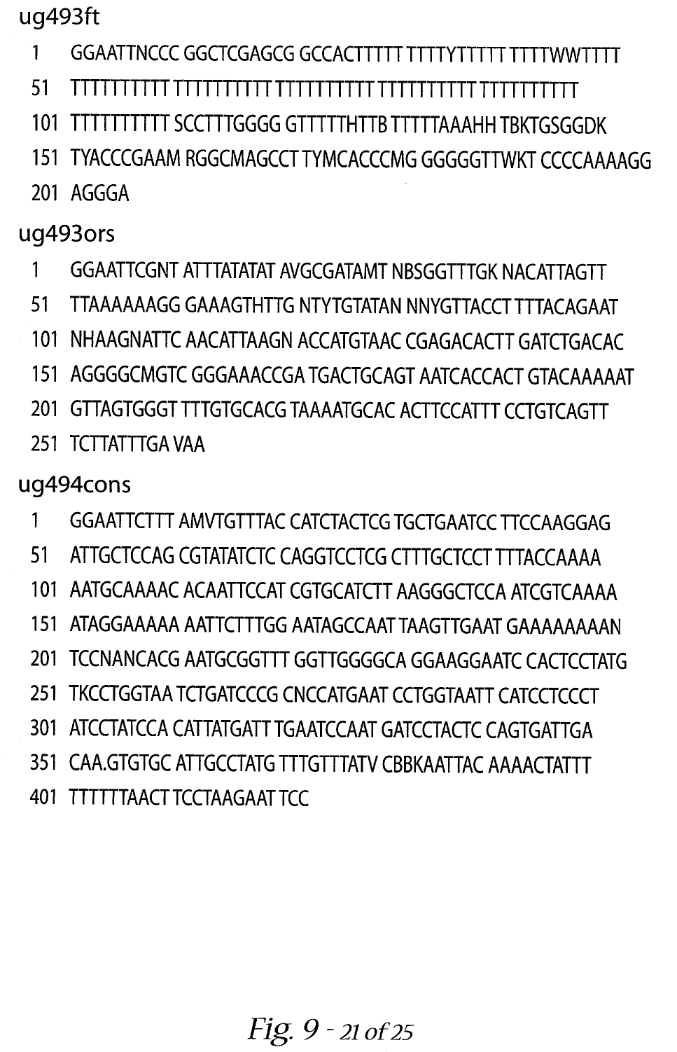

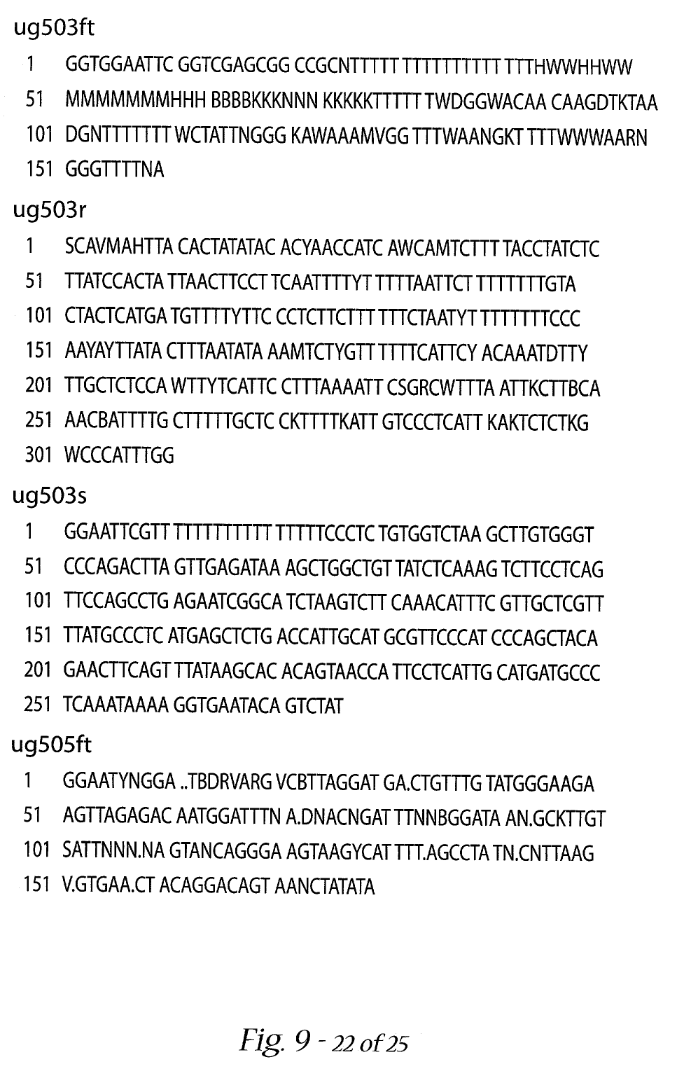

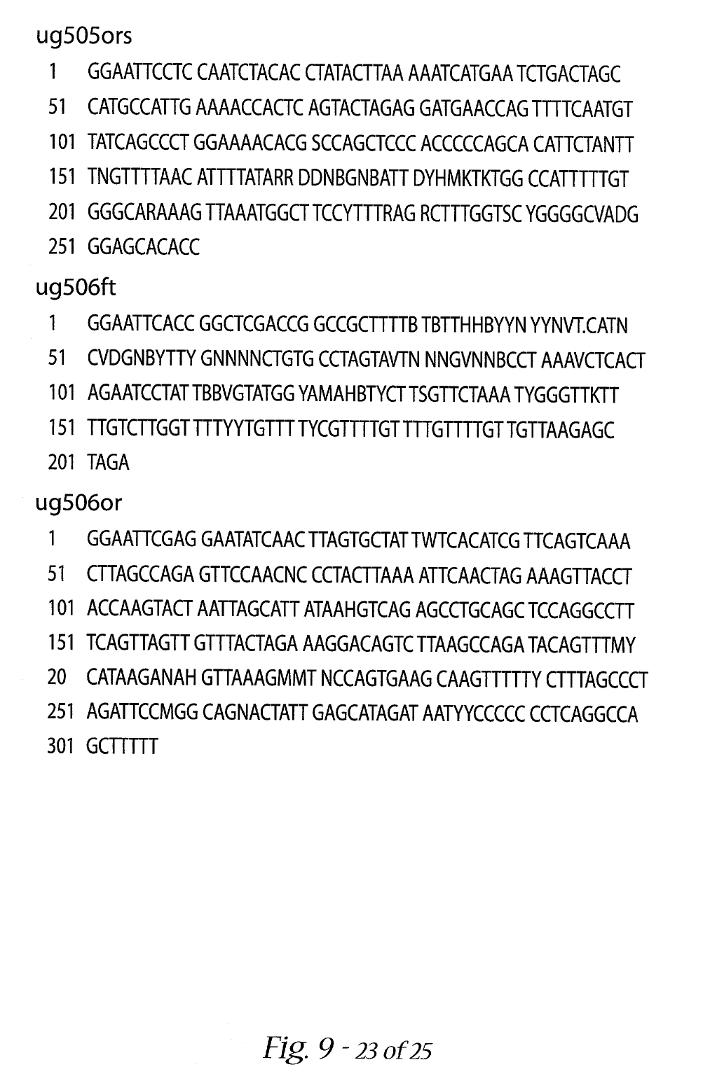

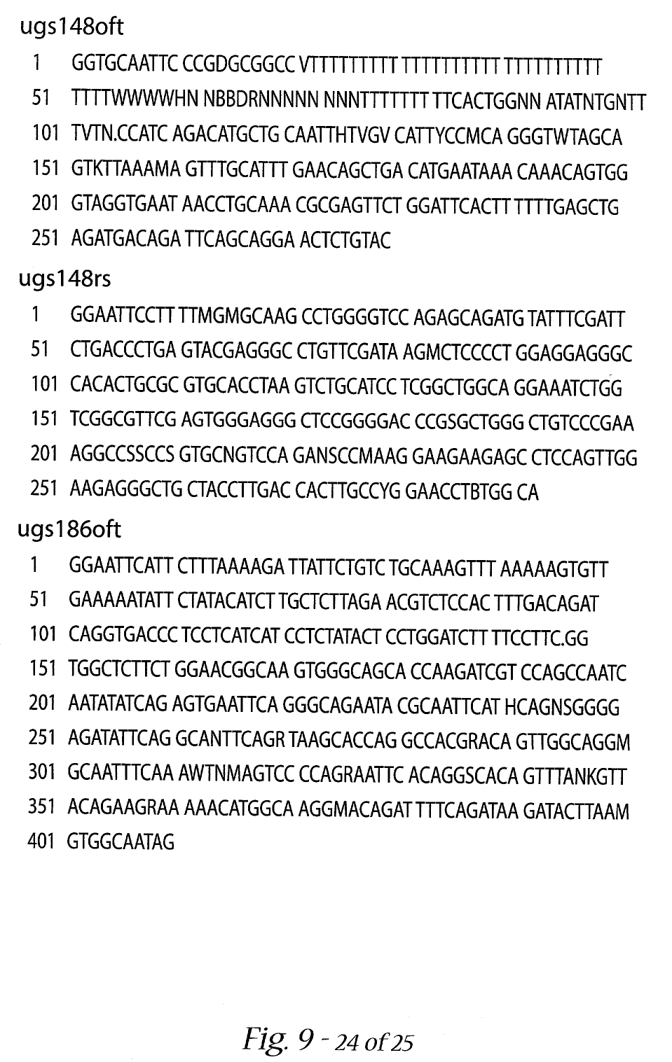

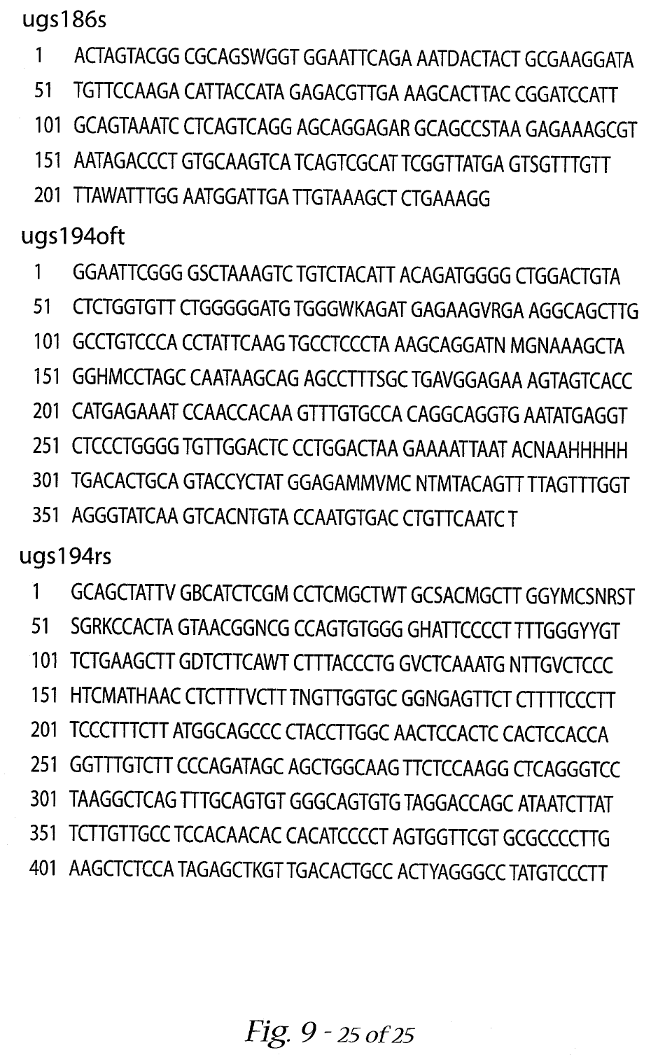

FIG. 9 depicts the additional consensus sequence of differentially expressed clones which have the following designations ug092 ft (SEQ ID NO:744); ug092ors (SEQ ID NO: 745); ug093f (SEQ ID NO:746); ug093ft (SEQ ID NO:747); ug106ft (SEQ ID NO:748); ug106ors (SEQ ID NO:749); ug120fmin (SEQ ID NO:750); ug120os (SEQ ID NO:751); ug254f (SEQ ID NO:752); ug254ors (SEQ ID NO:753); ug277f (SEQ ID NO:754); ug277ors (SEQ ID NO:755); ug277t (SEQ ID NO:756); ug291ft (SEQ ID NO:757); ug291ors (SEQ ID NO:758); ug307cons (SEQ ID NO:759); ug308f (SEQ ID NO:760); ug308o (SEQ ID NO: 761); ug308t (SEQ ID NO:762); ug311cons (SEQ ID NO:763); ug316cons (SEQ ID NO: 764); ug317cons (SEQ ID NO:765); ug320f (SEQ ID NO:766); ug320ors (SEQ ID NO:767); ug334ft (SEQ ID NO:768); ug334ors (SEQ ID NO:769); ug335ors (SEQ ID NO:770); ug335t (SEQ ID NO:771); ug353ft (SEQ ID NO:772); ug353ors (SEQ ID NO:773); ug354cons (SEQ ID NO:774); ug357ft (SEQ ID NO:775); ug357ors (SEQ ID NO:776); ug375cons (SEQ ID NO:777); ug371f (SEQ ID NO:778); ug440f (SEQ ID NO:779); ug440rs (SEQ ID NO:780); ug441ft (SEQ ID NO:781); ug441ors (SEQ ID NO:782); ug482ft (SEQ ID NO:783); ug093ors (SEQ ID NO:784); ug096f (SEQ ID NO:785); ug096ors (SEQ ID NO:786); ug101orsft (SEQ ID NO:787); ug102cons (SEQ ID NO:788); ug482ors (SEQ ID NO:789); ug484ft (SEQ ID NO:790); ug484ors (SEQ ID NO:791); ug485ors (SEQ ID NO:792); ug485t (SEQ ID NO:793); ug491ft (SEQ ID NO:794); ug491ors (SEQ ID NO:795); ug493ft (SEQ ID NO:796); ug493ors (SEQ ID NO:797); ug494cons (SEQ ID NO:798); ug503ft (SEQ ID NO:799); ug503r (SEQ ID NO:800); ug503s (SEQ ID NO:801); ug505ft (SEQ ID NO:802); ug505ors (SEQ ID NO:803); ug506ft (SEQ ID NO:804); ug506or (SEQ ID NO:805); ugs148oft (SEQ ID NO:806); ugs148rs (SEQ ID NO:807); ugs186oft (SEQ ID NO:808); ugs186s (SEQ ID NO:809); ugs194oft (SEQ ID NO:810); ugs194rs (SEQ ID NO:811).

Accordingly, the present invention relates to methods and compositions for the treatment and diagnosis of prostate disease, including but not limited to, prostatitis, and benign and malignant growth of the prostate gland. Specifically, fetal genes are identified and described which are differentially expressed in prostate disease states, relative to their expression in normal, or non-prostate disease states.

The present invention further relates to screening methods to identify compositions and their therapeutic use for the treatment of prostate disease, including but not limited to, prostatitis, and benign and malignant growth of the prostate gland. “Differential expression”, as used herein, refers to both quantitative as well as qualitative differences in the fetal genes' temporal and/or tissue expression patterns. Differentially expressed fetal genes may represent “fingerprint genes,” and/or “target genes.” “Fingerprint gene,” as used herein, refers to a differentially expressed fetal gene whose expression pattern may be utilized as part of a prognostic or diagnostic for prostate disease, including but not limited to, prostatitis, and benign and malignant growth of the prostate gland, disease evaluation, or which, alternatively, may be used in methods for identifying compounds useful for the treatment of prostate disease, including but not limited to, prostatitis, and benign and malignant growth of the prostate gland. “Target gene”, as used herein, refers to a differentially expressed gene involved in prostate disease, including but not limited to, prostatitis, and benign and malignant growth of the prostate gland such that modulation of the level of target gene expression or of target gene product activity may act to ameliorate a prostate disease condition. Compounds that modulate target gene expression or activity of the target gene product can be used in the treatment of prostate disease.

Further, “pathway genes” are defined via the ability of their products to interact with other gene products involved in the development of prostate disease, or the progression of prostate disease. Pathway genes may also exhibit target gene and/or fingerprint gene characteristics. Although the genes described herein may be differentially expressed with respect to prostate disease, and/or their products may interact with gene products important to prostate disease, the genes may also be involved in mechanisms important to additional prostate processes.

The invention further includes the products of such fingerprint, target, and pathway genes, as well as antibodies to such gene products. Furthermore, the engineering and use of cell- and animal-based models of prostate disease to which such gene products may contribute are also described.

The present invention encompasses methods for prognostic and diagnostic evaluation of prostate disease conditions, including but not limited to, prostatitis, and benign and malignant growth of the prostate gland, and for the identification of subjects exhibiting a predisposition to such conditions. Furthermore, the invention provides methods for evaluating the efficacy of drugs, and monitoring the progress of patients, involved in clinical trials for the treatment of prostate disease, including but not limited to, prostatitis, and benign and malignant growth of the prostate gland.

The invention also provides methods for the identification of compounds that modulate the expression of genes or the activity of gene products involved in prostate disease, including but not limited to, prostatitis, and benign and malignant growth of the prostate gland as well as methods for the treatment of prostate disease which may involve the administration of such compounds to individuals exhibiting prostate disease symptoms or tendencies.

The invention also provides methods for the identification of compounds that modulate the expression of genes or the activity of gene products involved in prostate disease, including but not limited to, prostatitis, and benign and malignant growth of the prostate gland.

The invention is based, in part, on systematic search strategies involving in vivo and in vitro prostate disease models, including but not limited to, prostatitis, and benign and malignant growth of the prostate gland, coupled with sensitive and high throughput gene expression assays. In contrast to approaches that merely evaluate the expression of a given gene product presumed to play a role in a prostate disease process, the search strategies and assays used herein permit the identification of all genes, whether known or novel, that are expressed or repressed in the prostate disease condition, as well as the evaluation of their temporal regulation and function during prostate disease progression. This comprehensive approach and evaluation permits the discovery of novel genes and gene products, as well as the identification of an array of genes and gene products (whether novel or known) involved in novel pathways that play a major role in prostate disease pathology. Thus, the invention allows one to define targets useful for diagnosis, monitoring, rational drug screening and design, and/or other therapeutic intervention for prostatic disease processes, including but not limited to, prostatitis, and benign and malignant growth of the prostate gland.

In the working examples described herein, novel human genes are identified that are demonstrated to be differentially expressed in different prostate disease states. The identification of these genes and the characterization of their expression in particular prostate disease states provide newly identified roles in prostate disease for these genes.

Specifically, ug311, and ug494 are two novel fetal urogenital sinus (UGS)-derived expressed sequence tags (ESTs) which represent novel genes that are each differentially regulated in the LNCaP progression prostate cancer model. The fetal gene-derived EST Ug311 is down-regulated in the aggressive, androgen independent PCa cell line, C4-2, whereas the fetal gene-derived EST ug494 is up-regulated in the C4-2 cell line compared to the LNCaP progression prostate cancer model cell line. The isolation and characterization of the fetal gene-derived EST Ug311 is presented in more detail in Example 1.

Accordingly, methods are provided for the diagnosis, monitoring in clinical trials, screening for therapeutically effective compounds, and treatment of prostate disease, including but not limited to, prostatitis, and benign and malignant growth of the prostate gland based upon the discoveries herein regarding the expression patterns of the fetal UGS-derived ESTs, ug311 and ug494.

The characteristic up-regulation of the ug494 fetal gene can be used to design prostate disease treatment strategies. For those up-regulated fetal genes that have a causative effect on the disease conditions, treatment methods can be designed to reduce or eliminate their expression, particularly in prostate cells. Alternatively, treatment methods include inhibiting the activity of the protein products of these fetal genes. For those up-regulated fetal genes that have a protective effect, treatment methods can be designed for enhancing the activity of the products of such fetal genes.

In either situation, detecting expression of these genes in excess of normal expression provides for the diagnosis of prostate disease. Furthermore, in testing the efficacy of compounds during clinical trials, a decrease in the level of the expression of these genes corresponds to a return from a disease condition to a normal state, and thereby indicates a positive effect of the compound. The prostate diseases that may be so diagnosed, monitored in clinical trials, and treated include, but are not limited to, prostatitis, and benign and malignant growth of the prostate gland.

The characteristic down-regulation of the ug311 fetal gene can also be used to design prostate disease treatment strategies. For those genes whose down-regulation has a pathogenic effect, treatment methods can be designed to restore or increase their expression, particularly in prostate cells. Alternatively, treatment methods include increasing the activity of the protein products of these fetal genes. For those fetal genes whose down-regulation has a protective effect, treatment methods can be designed for decreasing the amount or activity of the products of such fetal genes.

The invention encompasses methods for screening compounds and other substances for treating prostate disease symptoms, including but not limited to, prostatitis, and benign and malignant growth of the prostate gland, by assaying the ability of such compounds and other substances to modulate the expression of either the ug311 or ug494 fetal UGS-derived EST genes disclosed herein or activity of the protein products of the ug311 or ug494 fetal UGS-derived EST genes. The invention further encompasses methods for screening compounds and other substances such as steroids, anti-steroids, chemotherapeutics, including, for example, without limitation, compounds or analogs for nucleotide metabolism or nucleotide synthesis, radiation sensitizing agents, DNA repair enzymes or drugs targeting DNA repair, including, for example, without limitation, DNA topoisomerase inhibitors, potential Ku inhibitors or interacting proteins, and differentiation compounds, including, for example, without limitation, phenylacetate, and phenylbutyrate, and derivatives of such compounds, which may be used for treating human prostatic diseases and syndromes including, without limitation, prostatitis, and benign and malignant growth of the prostate gland, by assaying the ability of such compounds and other substances to modulate the expression of the target fetal genes disclosed herein or activity of the protein products of the target fetal genes. Such screening methods include, but are not limited to, assays for identifying compounds and other substances that interact with (e.g., bind to) the either the ug311 or ug494 fetal UGS-derived ESTs fetal gene products disclosed herein.

The data presented in Example 1, below, demonstrates the use of the prostate disease model of the invention to identify prostate disease target fetal genes.

In either situation, detecting expression of these fetal genes in below normal expression provides for the diagnosis of prostate disease. Furthermore, in testing the efficacy of compounds during clinical trials, an increase in the level of the expression of these fetal genes corresponds to a return from a disease condition to a normal state, and thereby indicates a positive effect of the compound. The prostate diseases that may be so diagnosed, monitored in clinical trials, and treated include, but are not limited to, prostatitis, and benign and malignant growth of the prostate gland.

In addition, the invention encompasses methods for treating prostate disease by administering compounds and other substances that modulate the overall activity of the target fetal gene products. Compounds and other substances can effect such modulation either on the level of target gene expression or target protein activity.

In order to identify differentially expressed genes, RNA, either total or mRNA, may be isolated from one or more tissues of the subjects utilized in the model systems such as those described earlier in this Section. RNA samples are obtained from tissues of experimental subjects and from corresponding tissues of control subjects. Any RNA isolation technique which does not select against the isolation of mRNA may be utilized for the purification of such RNA samples. See, for example, Sambrook et al., 1989, Molecular Cloning, A Laboratory Manual, Cold Spring Harbor Press, N.Y.; and Ausubel, F. M. et al., eds., 1987-1993, Current Protocols in Molecular Biology, John Wiley & Sons, Inc. New York, both of which are incorporated herein by reference in their entirety. Additionally, large numbers of tissue samples may readily be processed using techniques well known to those of skill in the art, such as, for example, the single-step RNA isolation process of Chomczynski, P. (1989, U.S. Pat. No. 4,843,155), which is incorporated herein by reference in its entirety.

Transcripts within the collected RNA samples which represent RNA produced by differentially expressed genes may be identified by utilizing a variety of methods which are well known to those of skill in the art. For example, differential screening (Tedder, T. F. et al., 1988, Proc. Natl. Acad. Sci. USA 85:208-212), subtractive hybridization (Hedrick, S. M. et al., 1984, Nature 308:149-153; Lee, S. W. et al., 1984, Proc. Natl. Acad. Sci. USA 88:2825), and, preferably, differential display (Liang, P., and Pardee, A. B., 1993, U.S. Patent No. 5,262,311, which is incorporated herein by reference in its entirety), may be utilized to identify nucleic acid sequences derived from genes that are differentially expressed.

Differential screening involves the duplicate screening of a cDNA library in which one copy of the library is screened with a total cell cDNA probe corresponding to the mRNA population of one cell type while a duplicate copy of the cDNA library is screened with a total cDNA probe corresponding to the mRNA population of a second cell type. For example, one cDNA probe may correspond to a total cell cDNA probe of a cell type derived from a control subject, while the second cDNA probe may correspond to a total cell cDNA probe of the same cell type derived from an experimental subject. Those clones which hybridize to one probe but not to the other potentially represent clones derived from genes differentially expressed in the cell type of interest in control versus experimental subjects.

Subtractive hybridization techniques generally involve the isolation of mRNA taken from two different sources, e.g., control and experimental tissue, the hybridization of the mRNA or single-stranded cDNA reverse-transcribed from the isolated mRNA, and the removal of all hybridized, and therefore double-stranded, sequences. The remaining non-hybridized, single-stranded cDNAs, potentially represent clones derived from genes that are differentially expressed in the two mRNA sources. Such single-stranded cDNAs are then used as the starting material for the construction of a library comprising clones derived from differentially expressed genes.

The differential display technique describes a procedure, utilizing the well known polymerase chain reaction (PCR; the experimental embodiment set forth in Mullis, K. B., 1987, U.S. Pat. No. 4,683,202) which allows for the identification of sequences derived from genes which are differentially expressed. First, isolated RNA is reverse-transcribed into single-stranded cDNA, utilizing standard techniques which are well known to those of skill in the art. Primers for the reverse transcriptase reaction may include, but are not limited to, oligo dT-containing primers, preferably of the reverse primer type of oligonucleotide described below. Next, this technique uses pairs of PCR primers, as described below, which allow for the amplification of clones representing a random subset of the RNA transcripts present within any given cell. Utilizing different pairs of primers allows each of the mRNA transcripts present in a cell to be amplified. Among such amplified transcripts may be identified those which have been produced from differentially expressed genes.

The reverse oligonucleotide primer of the primer pairs may contain an oligo dT stretch of nucleotides, preferably eleven nucleotides long, at its 5′ end, which hybridizes to the poly(A) tail of mRNA or to the complement of a cDNA reverse transcribed from an mRNA poly(A) tail. Second, in order to increase the specificity of the reverse primer, the primer may contain one or more, preferably two, additional nucleotides at its 3′ end. Because, statistically, only a subset of the mRNA derived sequences present in the sample of interest will hybridize to such primers, the additional nucleotides allow the primers to amplify only a subset of the mRNA derived sequences present in the sample of interest. This is preferred in that it allows more accurate and complete visualization and characterization of each of the bands representing amplified sequences.

The forward primer may contain a nucleotide sequence expected, statistically, to have the ability to hybridize to cDNA sequences derived from the tissues of interest. The nucleotide sequence may be an arbitrary one, and the length of the forward oligonucleotide primer may range from about 9 to about 13 nucleotides, with about 10 nucleotides being preferred. Arbitrary primer sequences cause the lengths of the amplified partial cDNAs produced to be variable, thus allowing different clones to be separated by using standard denaturing sequencing gel electrophoresis. PCR reaction conditions should be chosen which optimize amplified product yield and specificity, and, additionally, produce amplified products of lengths which may be resolved utilizing standard gel electrophoresis techniques. Such reaction conditions are well known to those of skill in the art, and important reaction parameters include, for example, length and nucleotide sequence of oligonucleotide primers as discussed above, and annealing and elongation step temperatures and reaction times.

The pattern of clones resulting from the reverse transcription and amplification of the mRNA of two different cell types is displayed via sequencing gel electrophoresis and compared. Differences in the two banding patterns indicate potentially differentially expressed genes.

Once potentially differentially expressed gene sequences have been identified via bulk techniques such as, for example, those described above, the differential expression of such putatively differentially expressed genes should be corroborated. Corroboration may be accomplished via, for example, such well known techniques as Northern analysis and/or RT-PCR.

Also, amplified sequences of differentially expressed genes obtained through, for example, differential display may be used to isolate full length clones of the corresponding gene. The full length coding portion of the gene may readily be isolated, without undue experimentation, by molecular biological techniques well known in the art. For example, the isolated differentially expressed amplified fragment may be labeled and used to screen a cDNA library. Alternatively, the labeled fragment may be used to screen a genomic library.

PCR technology may also be utilized to isolate full length cDNA sequences. As described above, the isolated, amplified gene fragments obtained through differential display have 5′ terminal ends at some random point within the gene and have 3′ terminal ends at a position preferably corresponding to the 3′ end of the transcribed portion of the gene. Once nucleotide sequence information from an amplified fragment is obtained, the remainder of the gene (i.e., the 5′ end of the gene, when utilizing differential display) may be obtained using, for example, RT-PCR.

In one embodiment of such a procedure for the identification and cloning of full length gene sequences, RNA may be isolated, following standard procedures, from an appropriate tissue or cellular source. A reverse transcription reaction may then be performed on the RNA using an oligonucleotide primer complimentary to the mRNA that corresponds to the amplified fragment, for the priming of first strand synthesis. Because the primer is anti-parallel to the mRNA, extension will proceed toward the 5′ end of the mRNA. The resulting RNA/DNA hybrid may then be “tailed” with guanines using a standard terminal transferase reaction, the hybrid may be digested with RNAase H, and second strand synthesis may then be primed with a poly-C primer. Using the two primers, the 5′ portion of the gene is amplified using PCR. Sequences obtained may then be isolated and recombined with previously isolated sequences to generate a full-length cDNA of the differentially expressed genes of the invention. For a review of cloning strategies and recombinant DNA techniques, see s, Sambrook et al., 1989, supra; and Ausubel et al., 1989, supra.

As used herein, “differentially expressed gene” (i.e. target and fingerprint gene) or “pathway gene” refers to (a) a gene containing at least one of the DNA sequences disclosed herein (as shown in FIG. 1 and FIG. 9), or contained in the UGS-derived ESTs listed in Tables 1-6; (b) any DNA sequence that encodes the amino acid sequence encoded by the DNA sequences disclosed herein (as shown in FIG. 1 and FIG. 9), contained in the ESTs listed in Tables 1-6, or contained within the coding region of the gene to which the DNA sequences disclosed herein (as shown in FIG. 1 and FIG. 9) or contained in the ESTs listed in Tables 1-6, belong; (c) any DNA sequence that hybridizes to the complement of the coding sequences disclosed herein (as shown in FIG. 1 and FIG. 9), contained in the ESTs listed in Tables 1-6, or contained within the coding region of the gene to which the DNA sequences disclosed herein (as shown in FIG. 1 and FIG. 9) or contained in the ESTs listed in Tables 1-6, under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & sons, Inc., New York, at p. 2.10.3) and encodes a fetal gene product functionally equivalent to a gene product encoded by the DNA sequences disclosed herein (as shown in FIG. 1 and FIG. 9) or a gene product encoded by sequences contained within the ESTs listed in Tables 1-6; and/or (d) any DNA sequence that hybridizes to the complement of the coding sequences disclosed herein, (as shown in FIG. 1 and FIG. 9) contained in the ESTs listed in Tables 1-6, or contained within the coding region of the gene to which DNA sequences disclosed herein (as shown in FIG. 1 and FIG. 9) or contained in the ESTs, listed in Tables 1-6, belong, under less stringent conditions, such as moderately stringent conditions, e.g., washing in 0.2×SSC/0.1% SDS at 42° C. (Ausubel et al., 1989, supra), yet which still encodes a functionally equivalent fetal gene product.

The invention also includes nucleic acid molecules, preferably DNA molecules, that hybridize to, and are therefore the complements of, the DNA sequences (a) through (c), in the preceding paragraph. Such hybridization conditions may be highly stringent or less highly stringent, as described above. In instances wherein the nucleic acid molecules are deoxyoligonucleotides (“oligos”), highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). These nucleic acid molecules may act as target gene antisense molecules, useful, for example, in target gene regulation and/or as antisense primers in amplification reactions of target gene nucleic acid sequences. Further, such sequences may be used as part of ribozyme and/or triple helix sequences, which are also useful for target gene regulation. Still further, such molecules may be used as components of diagnostic methods whereby the presence of a prostate disease-causing allele, may be detected.

The nucleotide sequences of the invention also include nucleotide sequences that have at least 65%, 70%, 75%, 80%, 85%, 90%, 95%, 98%, or more nucleotide sequence identity to a gene containing at least one of the DNA sequences disclosed herein (as shown in FIG. 1 and FIG. 9). The nucleotide sequences of the invention further include nucleotide sequences that encode polypeptides having at least 65%, 70%, 75%, 80%, 85%, 90%, 95%, 98%, or higher amino acid sequence identity to the polypeptides encoded by the nucleotide sequences disclosed herein (as shown in FIG. 1 and FIG. 9).

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993)Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res.25:3389-3402. Alternatively, PSI-Blast can be used to perform an iterated search which detects distant relationships between molecules (Id). When utilizing BLAST, Gapped BLAST, and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The invention also encompasses (a) DNA vectors that contain any of the foregoing coding sequences and/or their complements (i.e., antisense); (b) DNA expression vectors that contain any of the foregoing coding sequences operatively associated with a regulatory element that directs the expression of the coding sequences; and (c) genetically engineered host cells that contain any of the foregoing coding sequences operatively associated with a regulatory element that directs the expression of the coding sequences in the host cell. As used herein, regulatory elements include but are not limited to inducible and non-inducible promoters, enhancers, operators and other elements known to those skilled in the art that drive and regulate expression. The invention includes fragments of any of the DNA sequences disclosed herein.

In addition to the gene sequences described above, homologues of such sequences, as may, for example be present in other species, may be identified and may be readily isolated, without undue experimentation, by molecular biological techniques well known in the art. Further, there may exist genes at other genetic loci within the genome that encode proteins which have extensive homology to one or more domains of such gene products. These genes may also be identified via similar techniques.

For example, the isolated differentially expressed gene sequence may be labeled and used to screen a cDNA library constructed from mRNA obtained from the organism of interest. Hybridization conditions will be of a lower stringency when the cDNA library was derived from an organism different from the type of organism from which the labeled sequence was derived. Alternatively, the labeled fragment may be used to screen a genomic library derived from the organism of interest, again, using appropriately stringent conditions. Such low stringency conditions will be well known to those of skill in the art, and will vary predictably depending on the specific organisms from which the library and the labeled sequences are derived. For guidance regarding such conditions see, for example, Sambrook et al., 1989, Molecular Cloning, A Laboratory Manual, Cold Springs Harbor Press, N.Y.; and Ausubel et al., 1989, Current Protocols in Molecular Biology, Green Publishing Associates and Wiley Interscience, N.Y.

Further, a previously unknown differentially expressed or pathway gene-type sequence may be isolated by performing PCR using two degenerate oligonucleotide primer pools designed on the basis of amino acid sequences within the gene of interest. The template for the reaction may be cDNA obtained by reverse transcription of mRNA prepared from human or non-human cell lines or tissue known or suspected to express a differentially expressed or pathway gene allele.

The PCR product may be subcloned and sequenced to insure that the amplified sequences represent the sequences of a differentially expressed or pathway gene-like nucleic acid sequence. The PCR fragment may then be used to isolate a full length cDNA clone by a variety of methods. For example, the amplified fragment may be labeled and used to screen a bacteriophage cDNA library. Alternatively, the labeled fragment may be used to screen a genomic library.

PCR technology may also be utilized to isolate full length cDNA sequences. For example, RNA may be isolated, following standard procedures, from an appropriate cellular or tissue source. A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated. For a review of cloning strategies which may be used, see e.g., Sambrook et al., 1989, supra.

In cases where the differentially expressed or pathway gene identified is the normal, or wild type, gene, this gene may be used to isolate mutant alleles of the gene. Such an isolation is preferable in processes and disorders which are known or suspected to have a genetic basis. Mutant alleles may be isolated from individuals either known or suspected to have a genotype which contributes to prostate disease symptoms. Mutant alleles and mutant allele products may then be utilized in the therapeutic and diagnostic assay systems described below.

A cDNA of the mutant gene may be isolated, for example, by using PCR, a technique which is well known to those of skill in the art. In this case, the first cDNA strand may be synthesized by hybridizing an oligo-dT oligonucleotide to mRNA isolated from tissue known or suspected to be expressed in an individual putatively carrying the mutant allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant gene to that of the normal gene, the mutation(s) responsible for the loss or alteration of function of the mutant gene product can be ascertained.

Alternatively, a genomic or cDNA library can be constructed and screened using DNA or RNA, respectively, from a tissue known to or suspected of expressing the gene of interest in an individual suspected of or known to carry the mutant allele. The normal gene or any suitable fragment thereof may then be labeled and used as a probed to identify the corresponding mutant allele in the library. The clone containing this gene may then be purified through methods routinely practiced in the art, and subjected to sequence analysis as described above.

Additionally, an expression library can be constructed utilizing DNA isolated from or cDNA synthesized from a tissue known to or suspected of expressing the gene of interest in an individual suspected of or known to carry the mutant allele. In this manner, gene products made by the putatively mutant tissue may be expressed and screened using standard antibody screening techniques in conjunction with antibodies raised against the normal gene product, as described below. (For screening techniques, see, for example, Harlow, E. and Lane, eds., 1988, “Antibodies: A Laboratory Manual”, Cold Spring Harbor Press, Cold Spring Harbor.) In cases where the mutation results in an expressed gene product with altered function (e.g., as a result of a missense mutation), a polyclonal set of antibodies are likely to cross-react with the mutant gene product. Library clones detected via their reaction with such labeled antibodies can be purified and subjected to sequence analysis as described above.

In addition, differentially expressed and pathway gene products may include proteins that represent functionally equivalent gene products. Such an equivalent differentially expressed or pathway gene product may contain deletions, additions or substitutions of amino acid residues within the amino acid sequence encoded by the differentially expressed or pathway gene sequences described above but which result in a silent change, thus producing a functionally equivalent differentially expressed on pathway gene product. Amino acid substitutions may be made on the basis of similarity in polarity, charge, solubility, hydrophobicity, hydrophilicity, and/or the amphipathic nature of the residues involved.

For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Functionally equivalent”, as utilized herein, refers to a protein capable of exhibiting a substantially similar in vivo activity as the endogenous differentially expressed or pathway gene products encoded by the differentially expressed or pathway gene sequences described above. Alternatively, when utilized as part of assays such as those described below, “functionally equivalent” may refer to peptides capable of interacting with other cellular or extracellular molecules in a manner substantially similar to the way in which the corresponding portion of the endogenous differentially expressed or pathway gene product would.

The differentially expressed or pathway gene products may be produced by recombinant DNA technology using techniques well known in the art. Thus, methods for preparing the differentially expressed or pathway gene polypeptides and peptides of the invention by expressing nucleic acid encoding differentially expressed or pathway gene sequences are described herein. Methods which are well known to those skilled in the art can be used to construct expression vectors containing differentially expressed or pathway gene protein coding sequences and appropriate transcriptional/translational control signals. These methods include, for example, in vitro recombinant DNA techniques, synthetic techniques and in vivo recombination/genetic recombination. See, for example, the techniques described in Sambrook et al., 1989, supra, and Ausubel et al., 1989, supra. Alternatively, RNA capable of encoding differentially expressed or pathway gene protein sequences may be chemically synthesized using, for example, synthesizers. See, for example, the techniques described in “Oligonucleotide Synthesis”, 1984, Gait, M. J. ed., IRL Press, Oxford, which is incorporated by reference herein in its entirety.

Vectors, Host Cells, and Recombinant Expression

A variety of host-expression vector systems may be utilized to express the differentially expressed or pathway gene coding sequences of the invention. Such host-expression systems represent vehicles by which the coding sequences of interest may be produced and subsequently purified, but also represent cells which may, when transformed or transfected with the appropriate nucleotide coding sequences, exhibit the differentially expressed or pathway gene protein of the invention in situ. These include but are not limited to microorganisms such as bacteria (e.g., E. coli, B. subtilis) transformed with recombinant bacteriophage DNA, plasmid DNA or cosmid DNA expression vectors containing differentially expressed or pathway gene protein coding sequences; yeast (e.g. Saccharomyces, Pichia) transformed with recombinant yeast expression vectors containing the differentially expressed or pathway gene protein coding sequences; insect cell systems infected with recombinant virus expression vectors (e.g., baculovirus) containing the differentially expressed or pathway gene protein coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing differentially expressed or pathway gene protein coding sequences; or mammalian cell systems (e.g. COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the differentially expressed or pathway gene protein being expressed. For example, when a large quantity of such a protein is to be produced, for the generation of antibodies or to screen peptide libraries, for example, vectors which direct the expression of high levels of fusion protein products that are readily purified may be desirable. Such vectors include, but are not limited, to the E. coli expression vector pUR278 (Ruther et al., 1983, EMBO J. 2:1791), in which the differentially expressed or pathway gene protein coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene protein can be released from the GST moiety.

In a preferred embodiment, full length cDNA sequences are appended with in-frame BamHI sites at the amino terminus and EcoRI sites at the carboxyl terminus using standard PCR methodologies (Innis et al., 1990, supra) and ligated into the pGEX-2TK vector (Pharmacia, Uppsala, Sweden). The resulting cDNA construct contains a kinase recognition site at the amino terminus for radioactive labelling and glutathione 5-transferase sequences at the carboxyl terminus for affinity purification (Nilsson, et al., 1985, EMBO J.4:1075; Zabeau and Stanley, 1982, EMBO J 1:1217.

In an insect system, Autographa californica nuclear polyhedrosis virus (AcNPV) is used as a vector to express foreign genes. The virus grows in Spodoptera frugiperda cells. The differentially expressed or pathway gene coding sequence may be cloned individually into non-essential regions (for example the polyhedrin gene) of the virus and placed under control of an AcNPV promoter (for example the polyhedrin promoter). Successful insertion of differentially expressed or pathway gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (E.g., see Smith et al., 1983, J. Virol. 46:584; Smith, U.S. Pat. No. 4,215,051).

In mammalian host cells, a number of viral-based expression systems may be utilized. In cases where an adenovirus is used as an expression vector, the differentially expressed or pathway gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing differentially expressed or pathway gene protein in infected hosts. (E.g., See Logan & Shenk, 1984, Proc. Natl. Acad. Sci. USA 81:3655-3659). Specific initiation signals may also be required for efficient translation of inserted differentially expressed or pathway gene coding sequences. These signals include the ATG initiation codon and adjacent sequences. In cases where an entire differentially expressed or pathway gene, including its own initiation codon and adjacent sequences, is inserted into the appropriate expression vector, no additional translational control signals may be needed. However, in cases where only a portion of the differentially expressed or pathway gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544).

In a preferred embodiment, cDNA sequences encoding the full-length open reading frames are ligated into pCMVβ replacing the β-galactosidase gene such that cDNA expression is driven by the CMV promoter (Alam, 1990, Anal. Biochem. 188:245-254; MacGregor & Caskey, 1989, Nucl. Acids Res. 17:2365; Norton & Corrin, 1985, Mol. Cell. Biol. 5:281).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, etc.

For long-term, high-yield production of recombinant proteins, stable expression is preferred. For example, cell lines which stably express the differentially expressed or pathway gene protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the differentially expressed or pathway gene protein. Such engineered cell lines may be particularly useful in screening and evaluation of compounds that affect the endogenous activity of the differentially expressed or pathway gene protein.

A number of selection systems may be used, including but not limited to the herpes simplex virus thymidine kinase (Wigler, et al., 1977, Cell 11:223), hypoxanthine-guanine phosphoribosyltransferase (Szybalski & Szybalski, 1962, Proc. Natl. Acad. Sci. USA 48:2026), and adenine phosphoribosyltransferase (Lowy, et al., 1980, Cell 22:817) genes can be employed in tk-, hgprt- or aprt cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G418 (Colberre-Garapin, et al., 1981, J. Mol. Biol. 150:1); and hygro, which confers resistance to hygromycin (Santerre, et al., 1984, Gene 30:147) genes.

An alternative fusion protein system allows for the ready purification of non-denatured fusion proteins expressed in human cell lines (Janknecht, et al., 1991, Proc. Natl. Acad. Sci. USA 88:8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

When used as a component in assay systems such as those described below, the differentially expressed or pathway gene protein may be labeled, either directly or indirectly, to facilitate detection of a complex formed between the differentially expressed or pathway gene protein and a test substance. Any of a variety of suitable labeling systems may be used including but not limited to radioisotopes such as ¹²⁵I; enzyme labeling systems that generate a detectable colorimetric signal or light when exposed to substrate; and fluorescent labels.

Where recombinant DNA technology is used to produce the differentially expressed or pathway gene protein for such assay systems, it may be advantageous to engineer fusion proteins that can facilitate labeling, immobilization and/or detection.

Indirect labeling involves the use of a protein, such as a labeled antibody, which specifically binds to either a differentially expressed or pathway gene product. Such antibodies include but are not limited to polyclonal, monoclonal, chimeric, single chain, Fab fragments and fragments produced by an Fab expression library.

Described herein are methods for the production of antibodies capable of specifically recognizing one or more differentially expressed or pathway gene epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of a fingerprint, target, or pathway gene in a biological sample, or, alternatively, as a method for the inhibition of abnormal target gene activity. Thus, such antibodies may be utilized as part of prostate disease treatment methods, and/or may be used as part of diagnostic techniques whereby patients may be tested for abnormal levels of fingerprint, target, or pathway gene proteins, or for the presence of abnormal forms of such proteins.

For the production of antibodies to a differentially expressed or pathway gene, various host animals may be immunized by injection with a differentially expressed or pathway gene protein, or a portion thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

In a preferred embodiment, peptide sequences corresponding to amino sequences of target gene products are selected and submitted for synthesis and antibody production. Peptides are modified as described (Tam, J. P., 1988, Proc. Natl. Acad. Sci. USA 85:5409-5413; Tam, J. P., and Zavala, F., 1989, J. Immunol. Methods 124:53-61; Tam, J. P., and Lu, Y. A., 1989, Proc. Natl. Acad. Sci. USA 86:9084-9088), emulsified in an equal volume of Freund's adjuvant and injected into rabbits at 3 to 4 subcutaneous dorsal sites for a total volume of 1.0 ml (0.5 mg peptide) per immunization. The animals are boosted after 2 and 6 weeks and bled at weeks 4, 8, and 10. The blood is allowed to clot and serum is collected by centrifugation. The generation of polyclonal antibodies against the ug311 EST-derived gene products is described in detail below.

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, may be immunized by injection with differentially expressed or pathway gene product supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp.77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et. al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce differentially expressed or pathway gene-single chain antibodies. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include but are not limited to the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

Screening Assays for Compounds That Interact with the Target Gene Product and/or Modulate Target Gene Expression

The following assays are designed to identify compounds that bind to target gene products, bind to other cellular or extracellular proteins that interact with a target gene product, and interfere with the interaction of the target gene product with other cellular or extracellular proteins. Such compounds can act as the basis for amelioration of such prostate diseases, including, without limitation, prostatitis, and benign and malignant growth of the prostate gland by modulating the activity of the protein products of target genes. Such compounds may include, but are not limited to peptides, antibodies, or small organic or inorganic compounds. Such compounds may also include other cellular proteins. Methods for the identification of such cellular proteins are described below.

Compounds identified via assays such as those described herein may be useful, for example, in elaborating the biological function of the target gene product, and for ameliorating prostate disease including, without limitation, prostatitis, and benign and malignant growth of the prostate gland. In instances whereby a prostate disease condition results from an overall lower level of target gene expression and/or target gene product in a cell or tissue, compounds that interact with the target gene product may include compounds which accentuate or amplify the activity of the bound target gene protein. Such compounds would bring about an effective increase in the level of target gene product activity, thus ameliorating prostate disease symptoms.

In some cases, a target gene observed to be up-regulated under disease conditions may be exerting a protective effect. Compounds that enhance the expression of such up-regulated genes, or the activity of their gene products, would also ameliorate disease symptoms, especially in individuals whose target gene is not normally up-regulated.

In other instances mutations within the target gene may cause aberrant types or excessive amounts of target gene proteins to be made which have a deleterious effect that leads to prostate disease. Similarly, physiological conditions may cause an excessive increase in target gene expression leading to prostate disease. In such cases, compounds that bind target gene protein may be identified that inhibit the activity of the bound target gene protein. Assays for testing the effectiveness of compounds, identified by, for example, techniques such as those described above are discussed below.

In Vitro Screening Assays for Compounds That Bind to the Target Gene Product

In vitro systems may be designed to identify compounds capable of binding the target gene products of the invention. Such compounds may include, but are not limited to, peptides made of D-and/or L-configuration amino acids (in, for example, the form of random peptide libraries; see e.g., Lam, K. S. et al., 1991, Nature 354:82-84), phosphopeptides (in, for example, the form of random or partially degenerate, directed phosphopeptide libraries; see, e.g., Songyang, Z. et al., 1993, Cell 72:767-778), antibodies, and small organic or inorganic molecules. Compounds identified may be useful, for example, in modulating the activity of target gene proteins, preferably mutant target gene proteins, may be useful in elaborating the biological function of the target gene protein, may be utilized in screens for identifying compounds that disrupt normal target gene interactions, or may in themselves disrupt such interactions.

The principle of the assays used to identify compounds that bind to the target gene protein involves preparing a reaction mixture of the target gene protein and the test compound under conditions and for a time sufficient to allow the two components to interact and bind, thus forming a complex which can be removed and/or detected in the reaction mixture. These assays can be conducted in a variety of ways. For example, one method to conduct such an assay would involve anchoring the target gene or the test substance onto a solid phase and detecting target gene/test substance complexes anchored on the solid phase at the end of the reaction. In one embodiment of such a method, the target gene protein may be anchored onto a solid surface, and the test compound, which is not anchored, may be labeled, either directly or indirectly.

In practice, microtitre plates are conveniently utilized. The anchored component may be immobilized by non-covalent or covalent attachments. Non-covalent attachment may be accomplished simply by coating the solid surface with a solution of the protein and drying. Alternatively, an immobilized antibody, preferably a monoclonal antibody, specific for the protein may be used to anchor the protein to the solid surface. The surfaces may be prepared in advance and stored.

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the previously non-immobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for target gene product or the test compound to anchor any complexes formed in solution, and a labeled antibody specific for the other component of the possible complex to detect anchored complexes. Compounds such as those identified through assays described above which exhibit inhibitory activity may be used in accordance with the invention to ameliorate prostate disease symptoms. As discussed above, such molecules may include, but are not limited to small organic molecules, peptides, antibodies, and the like.

Pharmaceutical Preparations and Methods of Administration

The identified compounds that inhibit target gene expression, synthesis and/or activity can be administered to a patient at therapeutically effective doses to treat or ameliorate prostate disease, including, without limitation, prostatitis, and benign and malignant growth of the prostate gland. A therapeutically effective dose refers to that amount of the compound sufficient to result in amelioration of symptoms of prostate disease.

Effective Dose

Toxicity and therapeutic efficacy of such compounds can be determined by standard pharmaceutical procedures in cell cultures or experimental animals, e.g. for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Diagnosis of Prostate Disease Abnormalities

A variety of methods may be employed, utilizing reagents such as fingerprint gene nucleotide sequences described above and antibodies directed against differentially expressed and pathway gene peptides, as described above. Specifically, such reagents may be used, for example, for the detection of the presence of target gene mutations, or the detection of either over or under expression of target gene mRNA.

The methods described herein may be performed, for example, by utilizing pre-packaged diagnostic kits comprising at least one specific fingerprint gene nucleic acid or anti-fingerprint gene antibody reagent described herein, which may be conveniently used, e.g., in clinical settings, to diagnose patients exhibiting prostate disease symptoms, including, without limitation, symptoms due to prostatitis, and benign and malignant growth of the prostate gland or at risk for developing prostate disease, including, without limitation, prostatitis, and benign and malignant growth of the prostate gland.

Any cell type or tissue, preferably prostate tissue, including, for example, without limitation, prostatic fibroblasts, prostatic epithelial cells, prostatic neuroendocrine cells and other cells of basal origin, endothelial cells, smooth muscle cells, osteoblastic lineages, osteoclastic lineages, and other transitional epithelial cells which include transitional epithelium of the bladder and kidney, in which the fingerprint gene is expressed may be utilized in the diagnostics described below.

Detection of Fingerprint Gene Nucleic Acids

DNA or RNA from the cell type or tissue to be analyzed may easily be isolated using procedures which are well known to those in the art. Diagnostic procedures may also be performed “in situ” directly upon tissue sections (fixed and/or frozen) of patient tissue obtained from biopsies or resections, such that no nucleic acid purification is necessary. Nucleic acid reagents such as those described above may be used as probes and/or primers for such in situ procedures (see, for example, Nuovo, G. J., 1992, PCR in situ hybridization:protocols and applications, Raven Press, N.Y.).

Fingerprint gene nucleotide sequences, either RNA or DNA, may, for example, be used in hybridization or amplification assays of biological samples to detect prostate disease-related gene structures and expression. Such assays may include, but are not limited to, Southern or Northern analyses, single stranded conformational polymorphism analyses, in situ hybridization assays, and polymerase chain reaction analyses. Such analyses may reveal both quantitative aspects of the expression pattern of the fingerprint gene, and qualitative aspects of the fingerprint gene expression and/or gene composition. That is, such aspects may include, for example, point mutations, insertions, deletions, chromosomal rearrangements, and/or activation or inactivation of gene expression.

Preferred diagnostic methods for the detection of fingerprint gene-specific nucleic acid molecules may involve for example, contacting and incubating nucleic acids, derived from the cell type or tissue being analyzed, with one or more labeled nucleic acid reagents as are described above, under conditions favorable for the specific annealing of these reagents to their complementary sequences within the nucleic acid molecule of interest. Preferably, the lengths of these nucleic acid reagents are at least 9 to 30 nucleotides. After incubation, all non-annealed nucleic acids are removed from the nucleic acid:fingerprint molecule hybrid. The presence of nucleic acids from the fingerprint tissue which have hybridized, if any such molecules exist, is then detected. Using such a detection scheme, the nucleic acid from the tissue or cell type of interest may be immobilized, for example, to a solid support such as a membrane, or a plastic surface such as that on a microtitre plate or polystyrene beads. In this case, after incubation, non-annealed, labeled fingerprint nucleic acid reagents of the type described above are easily removed. Detection of the remaining, annealed, labeled nucleic acid reagents is accomplished using standard techniques well-known to those in the art. Alternative diagnostic methods for the detection of fingerprint gene specific nucleic acid molecules may involve their amplification, e.g., by PCR (the experimental embodiment set forth in Mullis, K. B., 1987, U.S. Pat. No. 4,683,202), ligase chain reaction (Barany, F., 1991, Proc. Natl. Acad. Sci. USA 88:189-193), self sustained sequence replication (Guatelli, J. C. et al., 1990, Proc. Natl. Acad. Sci. USA 87:1874-1878), transcriptional amplification system (Kwoh, D. Y et al., 1989, Proc. Natl. Acad. Sci. USA 86:1173-1177), Q-Beta Replicase (Lizardi, P. M. et al., 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment of such a detection scheme, a cDNA molecule is obtained from an RNA molecule of interest (e.g. by reverse transcription of the RNA molecule into cDNA). Cell types or tissues from which such RNA may be isolated include any tissue in which wild type fingerprint gene is known to be expressed, including, but not limited, to prostate tissue, endothelium, and/or smooth muscle. A fingerprint sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the fingerprint gene nucleic acid reagents described above. The preferred lengths of such nucleic acid reagents are at least 15-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

In addition to methods which focus primarily on the detection of one nucleic acid sequence, fingerprint profiles may also be assessed in such detection schemes. Fingerprint profiles may be generated, for example, by utilizing a differential display procedure, Northern analysis and/or RT-PCR. Any of the gene sequences described above may be used as probes and/or PCR primers for the generation and corroboration of such fingerprint profiles.

Detection of Fingerprint Gene Peptides

Antibodies directed against wild type or mutant fingerprint gene peptides, which are discussed above may also be used as prostate disease diagnostics and prognostics, as described, for example, herein. Such diagnostic methods, may be used to detect abnormalities in the level of fingerprint gene protein expression, or abnormalities in the structure and/or tissue, cellular, or subcellular location of fingerprint gene protein. Structural differences may include, for example, differences in the size, electronegativity, or antigenicity of the mutant fingerprint gene protein relative to the normal fingerprint gene protein.

Protein from the prostate tissue or cell type to be analyzed may easily be detected or isolated using techniques which are well known to those of skill in the art, including but not limited to western blot analysis. For a detailed explanation of methods for carrying out western blot analysis, see Sambrook et al, 1989, supra, at Chapter 18. The protein detection and isolation methods employed herein may also be such as those described in Harlow and Lane, for example, (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

Preferred diagnostic methods for the detection of wild type or mutant fingerprint gene peptide molecules may involve, for example, immunoassays wherein fingerprint gene peptides are detected by their interaction with an anti-fingerprint gene specific peptide antibody.

For example, antibodies, or fragments of antibodies, such as those described useful in the present invention may be used to quantitatively or qualitatively detect the presence of wild type or mutant fingerprint gene peptides. This can be accomplished, for example, by immunofluorescence techniques employing a fluorescently labeled antibody (see below) coupled with light microscopic, flow cytometric, or fluorimetric detection. Such techniques are especially preferred if the fingerprint gene peptides are expressed on the cell surface.

The antibodies (or fragments thereof) useful in the present invention may, additionally, be employed histologically, as in immunofluorescence or immunoelectron microscopy, for in situ detection of fingerprint gene peptides. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the fingerprint gene peptides, but also their distribution in the examined tissue. Using the present invention, those of ordinary skill will readily perceive that any of a wide variety of histological methods (such as staining procedures) can be modified in order to achieve such in situ detection.

Immunoassays for wild type or mutant fingerprint gene peptides typically comprise incubating a biological sample, such as a biological fluid, a tissue extract, freshly harvested cells, or cells which have been incubated in tissue culture, in the presence of a detectably labeled antibody capable of identifying fingerprint gene peptides, and detecting the bound antibody by any of a number of techniques well known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled fingerprint gene specific antibody. The solid phase support may then be washed with the buffer a second time to remove unbound antibody. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-wild type or mutant fingerprint gene peptide antibody may be determined according to well known methods. Those skilled in the art will be able to determine operative and optimal assay conditions for each determination by employing routine experimentation.

One of the ways in which the fingerprint gene peptide-specific antibody can be detectably labeled is by linking the same to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Imaging Prostate Disease Conditions

In some cases, differentially expressed gene products identified herein may be up-regulated under prostate disease conditions and expressed on the surface of the affected tissue including such gene products comprising those known receptor proteins, structural proteins, peptidases and proteinases, membrane proteins, growth factors and cytokines as identified in Tables 1-6, and the as yet uncharacterized cell surface molecules as found in the unknown categories of Tables 1-6. Such target gene products allow for the non-invasive imaging of damaged or diseased prostate tissue for the purposed of diagnosis and directing of treatment of prostate disease.

Monoclonal and polyclonal antibodies which specifically bind to such surface proteins can be used for the diagnosis of prostate disease by in vivo tissue imaging techniques. An antibody specific for a target gene product, or preferably an antigen binding fragment thereof, is conjugated to a label (e.g., a gamma emitting radioisotope) which generates a detectable signal and administered to a subject (human or animal) suspected of having prostate disease. After sufficient time to allow the detectably-labeled antibody to localize at the diseased or damaged tissue site (or sites), the signal generated by the label is detected by a photoscanning device. The detected signal is then converted to an image of the tissue. This image makes it possible to localize the tissue in vivo. This data can then be used to develop an appropriate therapeutic strategy.

Antibody fragments, rather than whole antibody molecules, are generally preferred for use in tissue imaging. Antibody fragments accumulate at the tissue(s) more rapidly because they are distributed more readily than are entire antibody molecules. Thus, an image can be obtained in less time than is possible using whole antibody. These fragments are also cleared more rapidly from tissues, resulting in a lower background signal. See, e., Haber et al., U.S. Pat. No. 4,036,945; Goldenberg et al., U.S. Pat. No. 4,331,647. The divalent antigen binding fragment (Fab′)₂ and the monovalent Fab are especially preferred. Such fragments can be prepared by digestion of the whole immunoglobulin molecule with the enzymes pepsin or papain according to any of several well known protocols. The types of labels that are suitable for conjugation to a monoclonal antibody for diseased or damaged tissue localization include, but are not limited to radiolabels (i.e. radioisotopes), fluorescent labels and biotin labels.

Among the radioisotopes that can be used to label antibodies or antibody fragments, gamma-emitters, positron-emitters, X-ray-emitters and fluorescence-emitters are suitable for localization. Suitable radioisotopes for labeling antibodies include Iodine-131, Iodine-123, Iodine-125, Iodine-126, Iodine-133, Bromine-77, Indium-111, Indium-113m, Gallium-67, Gallium-68, Ruthenium-95, Ruthenium-97, Ruthenium-103, Ruthenium-105, Mercury-107, Mercury-203, Rhenium-99m, Rhenium-105, Rhenium- 101, Tellurium-121m, Tellurium-122m, Tellurium-125m, Thulium-165, Thulium-167, Thulium-168, Technetium-99m and Fluorine-18. The halogens can be used more or less interchangeably as labels since halogen-labeled antibodies and/or normal immunoglobulins would have substantially the same kinetics and distribution and similar metabolism.

The gamma-emitters Indium-111 and Technetium-99m are preferred because these radiometals are detectable with a gamma camera and have favorable half lives for imaging in vivo. Antibody can be labelled with Indium-111 or Technetium-99m via a conjugated metal chelator, such as DTPA (diethlenetriaminepentaacetic acid). See Krejcarek et al., 1977, Biochem. Biophys. Res. Comm. 77:581; Khaw et al., 1980, Science 209:295; Gansow et al., U.S. Pat. No. 4,472,509; Hnatowich, U.S. Pat. No. 4,479,930, the teachings of which are incorporated herein by reference.

Fluorescent compounds that are suitable for conjugation to a monoclonal antibody include fluorescein sodium, fluorescein isothiocyanate, and Texas Red sulfonyl chloride. See, DeBelder & Wik, 1975, Carbohydrate Research 44:254-257. Those skilled in the art will know, or will be able to ascertain with no more than routine experimentation, other fluorescent compounds that are suitable for labeling monoclonal antibodies.

Gene Therapy

Gene therapy was originally conceived of as a specific gene replacement therapy for correction of heritable defects to deliver functionally active therapeutic genes into targeted cells. Initial efforts toward somatic gene therapy relied on indirect means of introducing genes into tissues, called ex vivo gene therapy, e.g., target cells are removed from the body, transfected or infected with vectors carrying recombinant genes and re-implanted into the body (“autologous cell transfer”). A variety of transfection techniques are currently available and used to transfer DNA in vitro into cells; including calcium phosphate-DNA precipitation, DEAE-Dextran transfection, electroporation, liposome mediated DNA transfer or transduction with recombinant viral vectors. Such ex vivo treatment protocols have been proposed to transfer DNA into a variety of different cell types including epithelial cells (U.S. Pat. No. 4,868,116; Morgan and Mulligan WO87/00201; Morgan et al., 1987, Science 237:1476-1479; Morgan and Mulligan, U.S. Pat. No. 4,980,286), endothelial cells (WO89/05345), hepatocytes (WO89/07136; Wolff et al., 1987, Proc. Natl. Acad. Sci. USA 84:3344-3348; Ledley et al, 1987 Proc. Natl. Acad. Sci. 84:5335-5339; Wilson and Mulligan, WO89/07136; Wilson et al., 1990, Proc. Natl. Acad. Sci. 87:8437-8441), fibroblasts (Palmer et al., 1987, Proc. Natl. Acad. Sci. USA 84:1055-1059; Anson et al., 1987, Mol. Biol. Med. 4:11-20; Rosenberg et al., 1988, Science 242:1575-1578; Naughton & Naughton, U.S. Pat. No. 4,963,489), lymphocytes (Anderson et al, U.S. Pat. No. 5,399,346; Blaese, R M. et al., 1995, Science 270:475-480) and hematopoietic stem cells (Lim, B. et al 1989, Proc. Natl. Acad. Sci. USA 86:8892-8896; Anderson et al., U.S. Pat. No. 5,399,346).

Direct in vivo gene transfer recently has been attempted with formulations of DNA trapped in liposomes (Ledley et al., 1987, J. Pediatrics 110:1), in proteoliposomes that contain viral envelope receptor proteins (Nicolau et al., 1983, Proc. Natl. Acad. Sci. U.S.A. 80:1068) and DNA coupled to a polylysine-glycoprotein carrier complex. In addition, “gene guns” have been used for gene delivery into cells (Australian Patent No. 9068389). It even has been speculated that naked DNA, or DNA associated with liposomes, can be formulated in liquid carrier solutions for injection into interstitial spaces for transfer of DNA into cells (Felgner, WO90/11092).

Perhaps, one of the greatest problems associated with currently devised gene therapies, whether ex vivo or in vivo, is the inability to transfer DNA efficiently into a targeted cell population and to achieve high level expression of the gene product in vivo. Viral vectors are regarded as the most efficient system, and recombinant replication-defective viral vectors have been used to transduce (i.e., infect) cells both ex vivo and in vivo. Such vectors have included retroviral, adenoviral, adeno-associated viral and herpes viral vectors. While highly efficient at gene transfer, the major disadvantages associated with the use of viral vectors include the inability of many viral vectors to infect non-dividing cells, problems associated with insertional mutagenesis, inflammatory reactions to the virus and potential helper virus production and/or production and transmission of harmful virus to other human patients. In addition to the low efficiency of most cell types to take up and express foreign DNA, many targeted cell populations are found in such low numbers in the body that the efficiency of presentation of DNA to the specific targeted cell types is diminished even further.

Retroviruses represent one class of viruses that have been studied extensively for use in gene therapy (Miller, A. D., 1990, Human Gene Ther. 1:5-14). Unfortunately, there are a number of disadvantages associated with retroviral use, including the random integration of retroviruses into the host genome, which often leads to insertional mutagenesis or the inadvertent activation of proto-oncogene expression due to the promoter activity associated with retroviral LTRs (long terminal repeats). Adeno-associated viruses (“AAV”) also have been studied as an alternative system for delivery of stable genetic information into a cell. These viruses have the desirable feature of potentially integrating in specific regions of the host genome. However, the usefulness of both retroviral and AAV vectors is limited by their inability to accept heterologous DNA fragments greater than 3-5 Kb, their inability to produce larger quantities of viral stocks and, in the case of retroviruses, their instability and inability to infect non-dividing cells.

Some viral constructs, including those using retroviruses, are capable of stabile transfection of host cells, leading to long-term transgene expression. Adenoviruses, to the contrary, insert their DNA episomally, leading to transient gene expression for 2-4 weeks. For some disease processes, such as cystic fibrosis, permanent transgene expression clearly would be required (Cook S D, et al., 1996, Clinical Orthopedics and Related Research, 324:29-38). Thus, retroviral or adeno-associated viral vectors, which are capable of integrating into the hosts's genome, would be desirable for the treatment of these disease processes. For other diseases, wherein transgenes encode, for example, growth factors, transient expression may be advantageous, since prolonged gene expression could lead to serious side-effects. In these cases, a non-integrating viral vector, such as adenovirus, would be preferred.

Adenovirus Based Vectors

Adenovirus is a large, non-enveloped virus consisting of a dense protein capsid and a large linear (36 kb) double stranded DNA genome. Adenovirus infects a variety of both dividing and non-dividing cells, gaining entry by receptor-mediated uptake into endosomes, followed by internalization. After uncoating, the adenovirus genome expresses a large number of different gene products that are involved in viral replication, modification of host cell metabolism and packaging of progeny viral particles. Three adenovirus gene products are essential for replication of viral genomes: (1) the terminal binding protein which primes DNA replication, (2) the viral DNA polymerase and (3) the DNA binding protein (reviewed in Tamanoi and Stillman, 1983, Immunol. 109:75-87). In addition, processing of the terminal binding protein by the adenovirus 23 kDa L3 protease is required to permit subsequent rounds of reinfection (Stillman et al., 1981, Cell, 23:497-508) as well as to process adenovirus structural proteins, permitting completion of self-assembly of capsids (Bhatti and Weber, 1979, Virology, 96:478-485).

Packaging of nascent adenovirus particles takes place in the nucleus, requiring both cis-acting DNA elements and trans-acting viral factors, the latter generally construed to be a number of viral structural polypeptides. Packaging of adenoviral DNA sequences into adenovirus capsids requires the viral genomes to possess functional adenovirus encapsidation signals, which are located in the left and right termini of the linear viral genome (Hearing et al., 1987, J. Virol.61:2555-2558). Additionally, the packaging sequence must reside near the ends of the viral genome to function (Hearing et al., 1987, J. Virol. 61:2555-2558; Grable and Hearing, 1992, J. Virol., 66:723-731). The E1A enhancer, the viral replication origin and the encapsidation signal compose the duplicated inverted terminal repeat (ITR) sequences located at the two ends of adenovirus genomic DNA. The replication origin is defined loosely by a series of conserved nucleotide sequences in the ITR which must be positioned close to the end of the genome to act as a replication-priming element (reviewed in Challberg and Kelly, 1989, Biochem, 58:671-717; Tamanoi and Stillman, 1983, Immunol. 109:75-87). As shown by several groups, the ITRs are sufficient to confer replication to a heterologous DNA in the presence of complementing adenovirus functions. Adenovirus “mini-chromosomes” consisting of the terminal ITRs flanking short linear DNA fragments (in some cases non-viral DNAs) were found to replicate in vivo at low levels in the presence of infecting wild-type adenovirus, or in vitro at low levels in extracts prepared from infected cells (e.g., Hay et al., 1984, J. Mol. Biol. 175:493-510; Tamanoi and Stillman, 1983, Immunol. 109:75-87). Evidence for trans-packaging of mini-chromosomes was not reported in these or any later studies concerned with mechanisms of adenovirus DNA replication, and it is unlikely that packaging occurred for several reasons. First, the replicated molecules were quite small and they were not expressed at levels high enough to compete for packaging. Second, no selection for trans-packaging was employed, making it inconceivable that the heterologously replicated molecules could compete for packaging against wild-type adenovirus genomes.

The expression of foreign genes in “replication-defective” adenoviruses (deleted of region E1) has been exploited for a number of years in many labs, and a variety of published reports describe several different approaches often used in constructing these vectors (Vernon et al., 1991, J. Gen. Virol., 72:1243-1251; Wilkinson and Akrigg, 1992, Nuc. Acids Res., 20:2233-2239; Eloit et al., 1990, J. Gen. Virol., 71:2425-2431; Johnson, 1991; Prevec et al., 1990, J. Infect. Dis., 161:27-30; Haj-Ahmad and Graham, 1986, J. Virol., 57:267-274; Lucito and Schneider, 1992, J. Virol., 66:983-991; reviewed in Graham and Prevec, 1992, Butterworth-Heinemann, 363-393). In general, replication-defective viruses are produced by replacing part, or all, of essential region E1 with a heterologous gene of interest, either by direct ligation to vial genomes in vitro, or by homologous recombination within cells in vivo (procedures reviewed in Berkner, 1992, Curr. Topics Micro. Immunol., 158:39-66). These procedures all produce adenovirus vectors that replicate in complementing cell lines such as 293 cells which provide the E1 gene products in trans. Replication competent adenovirus vectors also have been described that have the heterologous gene of interest inserted in place of non-essential region E3 (e.g., Haj-Ahmad and Graham, 1986, J. Virol. 57:267-274), or between the right ITR and region E4 (Saito et al., 1985, J. Virol., 54:711-719). In both, replication defective viruses and replication competent viruses, the heterologous gene of interest is incorporated into viral particles by packaging of the recombinant adenovirus genome.

Some viral constructs, including those using retroviruses, are capable of stable transfection of host cells, leading to long-term transgene expression. Adenoviruses, to the contrary, insert their DNA episomally, leading to transient gene expression for 2-4 weeks. For some disease processes, such as cystic fibrosis and osteoporosis, permanent transgene expression clearly would be required (Cook S D, et al., 1996, Clinical Orthopedics and Related Research, 324:29-38). Thus, retroviral or adeno-associated viral vectors, which are capable of integrating into the hosts's genome, would be desirable for the treatment of these disease processes. For other diseases, wherein transgenes encode, for example, growth factors, transient expression may be advantageous, since prolonged gene expression could lead to serious side-effects. In these cases, a non-integrating viral vector, such as adenovirus, would be preferred.

One may obtain the DNA segment encoding the protein of interest using a variety of molecular biological techniques, generally known to those skilled in the art. For example, cDNA or genomic libraries may be screened using primers or probes with sequences based on the known nucleotide sequences. Polymerase chain reaction (PCR) also may be used to generate the DNA fragment encoding the protein of interest. Alternatively, the DNA fragment may be obtained from a commercial source.

The DNA encoding the translational or transcriptional products of interest may be engineered recombinantly into a variety of vector systems that provide for replication of the DNA in large scale for the preparation of the viral vectors of the invention. These vectors can be designed to contain the necessary elements for directing the transcription and/or translation of the DNA sequence taken up by the bone cells at the repair site in vivo.

Methods which are well known to those skilled in the art can be used to construct expression vectors containing the protein coding sequence operatively associated with appropriate transcriptional/translational control signals. These methods include in vitro recombinant DNA techniques, and synthetic techniques. See, for example, the techniques described in Sambrook, et al., 1992, Molecular Cloning, A Laboratory Manual, Cold Spring Harbor Laboratory, N.Y. and Ausubel et al., 1989, Current Protocols in Molecular Biology, Greene Publishing Associates & Wiley Interscience, N.Y.

The genes encoding the proteins of interest may be associated operatively with a variety of different promoter/enhancer elements. The expression elements of these vectors may vary in their strength and specificities. Depending on the host/vector system utilized, any one of a number of suitable transcription and translation elements may be used. The promoter may be in the form of the promoter which is associated naturally with the gene of interest. Alternatively, the DNA may be positioned under the control of a recombinant or heterologous promoter, i.e., a promoter that is not associated normally with that gene. For example, tissue specific promoter/enhancer elements may be used to regulate the expression of the transferred DNA in specific cell types. Examples of transcriptional control regions that exhibit tissue specificity which have been described and could be used, include, but are not limited to: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:42S-51S); insulin gene control region which is active in pancreatic beta cells (Hanahan, 1985, Nature 315:115-122); immunoglobulin gene control region which is active in lymphoid cells (Grosschedl et al., 1984, Cell 38:647-658; Adams et al., 1985, Nature 318:533-538; Alexander et al., 1987, Mol. Cell. Biol. 7:1436-1444); albumin gene control region which is active in liver (Pinkert et al., 1987, Genes and Devel. 1:268-276); alpha-fetoprotein gene control region which is active in liver (Krumlauf et al., 1985, Mol. Cell. Biol. 5:1639-1648; Hammer et al., 1987, Science 235:53-58); alpha-1-antitrypsin gene control region which is active in liver (Kelsey et al., 1987, Genes and Devel. 1:161-171); beta-globin gene control region which is active in myeloid cells (Magram et al., 1985, Nature 315:338-340; Kollias et al., 1986, Cell 46:89-94); myelin basic protein gene control region which is active in oligodendrocyte cells in the brain (Readhead et al., 1987, Cell 48:703-712); myosin light chain-2 gene control region which is active in skeletal muscle (Shani, 1985, Nature 314:283-286) and gonadotropic releasing hormone gene control region which is active in the hypothalamus (Mason et al., 1986, Science 234:1372-1378). Promoters isolated from the genome of viruses that grow in mammalian cells, other than the CMV promoter, (e.g., RSV, vaccinia virus 7.5K, SV40, HSV, adenoviruses MLP, and MMTV LTR promoters) may be used, as well as promoters produced by recombinant DNA or synthetic techniques.

The use of tissue specific promoters to drive therapeutic gene expression would decrease further a toxic effect of the therapeutic gene on neighboring normal cells when virus-mediated gene delivery results in the infection of the normal cells. This would be important especially in diseases where systemic administration could be utilized to deliver a therapeutic vector throughout the body, while maintaining transgene expression to a limited and specific number of cell types. Moreover, since many growth factors, such as TGF-β, have pleiotropic effects, numerous, harmful side effects likely would be exhibited if the growth factor genes are expressed in all cells.

In some instances, the promoter elements may be constitutive or inducible promoters and can be used under the appropriate conditions to direct high level or regulated expression of the gene of interest. Expression of genes under the control of constitutive promoters does not require the presence of a specific substrate to induce gene expression and will occur under all conditions of cell growth. In contrast, expression of genes controlled by inducible promoters is responsive to the presence or absence of an inducing agent. For example, if a cell is stably transfected with a therapeutic, inducible transgene, its expression could be controlled over the life-time of the individual.

Specific initiation signals also are required for sufficient translation of inserted protein coding sequences. These signals include the ATG initiation codon and adjacent sequences. In cases where the entire coding sequence, including the initiation codon and adjacent sequences, are inserted into the appropriate expression vectors, no additional translational control signals may be needed. However, in cases where only a portion of the coding sequence is inserted, exogenous translational control signals, including the ATG initiation codon, must be provided. Furthermore, the initiation codon must be in phase with the reading flame of the protein coding sequences to ensure translation of the entire insert. These exogenous translational control signals and initiation codons can be of a variety of origins, both natural and synthetic. The efficiency and control of expression may be enhanced by the inclusion of transcription attenuation sequences, enhancer elements, etc.

In addition to DNA sequences encoding therapeutic proteins of interest, the scope of the present invention includes the use of ribozymes or antisense DNA molecules that may be transferred into mammalian cells. Such ribozymes and antisense molecules may be used to inhibit the translation of RNA encoding proteins of genes that promote the prostate disease process.

The expression of antisense RNA molecules will act directly to block the translation of mRNA by binding to targeted mRNA and preventing protein translation. The expression of ribozymes, which are enzymatic RNA molecules capable of catalyzing the specific cleavage of RNA, also may be used to block protein translation. The mechanism of ribozyme action involves sequence specific hybridization of the ribozyme molecule to complementary target RNA, followed by an endonucleolytic cleavage. Within the scope of the invention are engineered hammerhead motif ribozyme molecules that specifically and efficiently catalyze endonucleolytic cleavage of RNA sequences. RNA molecules may be generated by transcription of DNA sequences encoding the RNA molecule.

It also is within the scope of the invention that multiple genes, combined on a single genetic construct under control of one or more promoters, or prepared as separate constructs of the same or different types, may be used. Thus, an almost endless combination of different genes and genetic constructs may be employed. Certain gene combinations may be designed to, or their use may otherwise result in, achieving synergistic effects in amelioration of prostate disease, and any and all such combinations are intended to fall within the scope of the present invention. Indeed, many synergistic effects have been described in the scientific literature, so that one of ordinary skill in the art readily would be able to identify likely synergistic gene combinations, or even gene-protein combinations. It will also be appreciated to those skilled in the art that the invention can be performed within a wide range of equivalent parameters of composition, concentration, modes of administration, and conditions without departing from the spirit or scope of the invention or any embodiment thereof.

Having now fully described the invention, the same will be more readily understood by reference to specific examples which are provided by way of illustration, and are not intended to be limiting of the invention, unless herein specified.

### EXAMPLE 1

Current staging and prognostic modalities for human prostate cancer are inadequate. Furthermore, our comprehension of the genetics of prostate carcinogenesis is lacking, although several genetic and epigenetic factors have been identified that correlate with the development of a more aggressive neoplastic phenotype. In the human, mesenchymal-epithelial interaction maintains the functional integrity of the adult prostate gland. Prior investigations in this laboratory have demonstrated that fetal mesenchyme has the capacity to initiate glandular overgrowth of the adult rodent prostate (McKinnell et al., New York:Plenum Press, 1989; Sikes et al., Biology of Reproduction. 43:353-62, 1990), reduce anaplasia in the Dunning prostatic adenocarcinoma model (Chung et al., Prostate. 17:165-74, 1990; Hayashi et al., Cancer Research. 50:4747-54, 1990), and induce the differentiation of androgen receptor-deficient urogenital sinus epithelium (UGE) into functional prostate tissue (Sikes et al., Biology of Reproduction. 43:353-62,1990; Chung et al.,Molecular Biology Reports. 23:13-19,1996; Bissell et al., The Journal of Theoretical Biology. 99:31-68, 1982).

Prostatic carcinogenesis may be explained by aberrant instructive influences derived from its underlying stroma, as the microenvironment surrounding the cancer epithelium has been demonstrated to determine tumor growth and malignant potential (Drews et al., Cell. 10:401-404,1977; Franks et al., The Journal of Pathology. 100:113-120,1970). Consequently, it is believed that abnormal prostate growth and carcinogenesis may result from abnormalities in the constituents of the stromal-epithelial milieu. The inductive role of stroma has been demonstrated in a wide variety of glandular tissues during embryonic development, including the prostate (Bissell et al., The Journal of Theoretical Biology. 99:31-68, 1982; McNeal, Investigative Urology. 15:340-5,1978; Cunha et al., Journal of Steroid Biochemistry. 14:1317-24, 1981; Cunha et al., Biology of Reproduction. 22:19-42, 1980; Chung et al., Prostate. 4:503-11, 1983; Cunhaetal., Endocrine Reviews.8:338-62,1987). Prostatic proliferation in the adult may result from a reawakening of dormant embryonic growth elements present in the prostatic stroma (Pierce, N.J.: Prentiss-Hall, Inc., 1978). It has been demonstrated that fetal urogenital sinus mesenchyme (UGM), a fetal form of prostatic stroma, is inductive and can redirect prostatic epithelial growth and differentiation (Sikes et al., Biology of Reproduction. 43:353-62, 1990; Chung et al., Biology of Reproduction. 31:155-163, 1984; Gleave et al., Cancer Research. 51:3753-61, 1991). Marked growth and expression of tissue-specific secretory proteins can be induced when fetal UGM is recombined with either fetal or adult prostate epithelium (Chung, Cancer Surveys. 23:33-42,1995; Evans, The British Journal of Cancer. 68:1051 -1060, 1993) or when it is implanted directly into the adult prostate gland (Han et al., Carcinogenesis. 16:951-954, 1995). Implanted fetal mesenchyme can induce differentiation and growth of adult rat urogenital cells (Chung et al., Prostate. 17:165-74, 1990; Hayashi et al., Cancer Research. 50:4747-54, 1990). Recombinants of androgen receptor deficient fetal mesenchyme with either fetal or adult epithelium failed to produce appropriate cytodifferentiation when recombined with fetal UGM lacking the androgen receptor (derived from testicular feminization, Tfm/y, fetuses) (Sikes et al., Biology of Reproduction. 43:353-62, 1990). This further supports the contention that paracrine mediators between stroma and epithelium are prerequisite for prostate growth and morphogenesis.

Inductive influences from stroma to prostatic epithelial differentiation can be classified as either directive or permissive, depending upon the sources of embryonic epithelium and the age of both the inductive and responsive fetal tissue (Cunha et al., Recent Progress in Hormone Research. 39:559-98, 1983). Thereafter, the ultimate growth potential of the embryonic and adult prostatic epithelium in tissue recombinants or in situ will be dictated by the presence and origin of inductive stroma. By varying the amount of embryonic stroma used in the construction of tissue recombinants (Evans, The British Journal of Cancer. 68:1051-1060, 1993) or by inserting fetal UGM directly into the adult prostate (Han et al., Carcinogenesis. 16:951-954, 1995), it has been shown that the growth potential of prostatic epithelium is dictated entirely by the amount of UGM present in either tissue recombinants or in the induced chimeric adult gland. Hence, mesenchymal agents can induce normal and neoplastic prostate growth and differentiation. This implies that the adult epithelium is capable of responding to a fetal inducer that is no longer present in normal prostate tissue. Furthermore, prostate carcinogenesis mimics a reversion to a more developmentally primitive state. Therefore, the differential expression of prostate-fetal genes may direct neoplastic transformation or at least identify when a clonal population has undergone such transformation.

The temporal involvement of steroid hormones and growth factors is paramount to prostate development. Prostate growth and differentiation is tightly regulated by androgens and is influenced by a number of soluble peptide growth factors and their receptors (Sokoloffet al., Cancer. 77:1862-1872, 1996). A close reciprocal association between stromal and epithehal tissues also has a fundamental role in normal, benign, and malignant prostate development. Mesenchymal and epithelial differentiation depends upon the stimulatory effects of dihydrotestosterone, inductive growth factors and peptides, and embryonic factors (Sokoloff et al., Cancer. 77:1862-1872, 1996). The combination of epidermal growth factor, transforming growth factor-β, insulin growth factor, and gonadotropin can induce differentiation of reproductive cells. Other studies have demonstrated that many of the properties associated with tumor progression and metastasis in hormone-refractory prostate cancer cell lines can be altered after treatment with cytokines (Ritchie et al., Endocrinology. 138:1145-1150, 1997; Ausubel et al., Preparing DNA from small-scale liquid lysates. In: K. Janssen (ed.) Current protocols in molecular biology., Vol. 1, pp. Section 1.13.7. New York: John Wiley and Sons, Inc., 1994). These studies found that suppression of prostate cancer cell growth correlated with the down regulation of oncogene, suppressor gene, growth factor, and adhesion molecule gene expression. Currently, there are no fetal-prostate markers described in prostate cancer for use as either diagnostic or prognostic markers. Therefore, this study describes the isolation of novel fetal prostate-derived genes for the purpose of developing prostatic markers. Further, for the first time fetal prostate genes are shown to be (re)expressed in prostate cancer cell lines.

The hypothesis to be tested in the present study is that fetal UGS-derived gene (re)expression or loss is important in the development and progression of prostate cancer. Furthermore, these genes encode oncofetal proteins that can serve as diagnostic, prognostic and therapeutic targets for use in the management of human prostate cancer. This study presents the cloning, characterization, and examination of the expression and possible role of a single differentially-expressed fetal UGS-derived gene, UG311, in cell lines and human prostate cancer specimens.

Aim

To clone and characterize the full-length cDNA corresponding to the differentially expressed urogenital sinus-derived expressed sequence tags, UG311, from LNCaP or C4-2 lambda gt11 cDNA libraries or by 5′- and 3′-RACE.a) Urogenital sinus (UGS)-derived expressed sequence tags will be used as probes to identify homologous phage inserts in LNCaP or C4-2 cDNA libraries. Overlapping contigs will be assembled as required. b) Alternatively, UGS-derived EST homologs will be cloned using 5′-3′- rapid amplification of cDNA ends (RACE) using LNCaP and C4-2 as mRNA as starting materials. Sequences obtained will be compared to those from lambda phage inserts and a closely related GenBank sequence nmt55.

Experimental Approach

The original UG311 insert was sequenced bidirectionally and found to contain an insert of 682 bp. The GenBank analysis of this insert revealed -98% homology to a drosophila protein, nonA^{diss} and the putative mammalian homologue NonO (Mahana et al., Journal of Immunological Methods. 161:187-192,1993). NonA/NonO has been described as a non-POU domain octamer-binding protein. Octamer binding proteins (OBP) are transcription factors that regulate the expression of a wide range of genes. This occurs from both the direct interaction of the OBP with DNA as well as the OBP's interaction with other transcription factors to determine the final modulation of a particular gene's transcriptional rate (Harlow et al., Antibodies:A laboratory Manual., pp. 726. New York:Cold Spring Harbor Laboratory, 1988; Sikes et al., Cancer Research. 52:3174-81, 1992).

Classical OBPs, those that contain a POU-domain, have family members that are ubiquitously expressed as well as those that have tissue-restricted expression patterns (Zhau et al., The Prostate. 28:73-83, 1996; Marengo et al., Molecular Carcinogenesis. In Press:, 1997). Those with tissue specific expression have been shown to be important in the development and maintenance of that cell phenotype (Zhau et al., The Prostate. 28:73-83, 1996; Marengo et al., Molecular Carcinogenesis. In Press:, 1997). The ubiquitous NonO/NonA mRNA was shown to have an open reading frame of 1418 bp encoded by a 2.4 kb cDNA (Mahana et al., Journal of Immunological Methods. 161:187-192, 1993). RNA blot analysis indicated ubiquitous expression of a 1.6 kb RNA with a band present also in mouse prostate tissue. The largest and tissue-specific mRNA described for NonO/NonA was 3.8 kb found exclusively in the retina. RNA blot analysis using UG311 as a probe on prostate cancer cell line RNA (FIG. 3) gave an initial mRNA signal corresponding to 3.2 kb. This data implied that either UG311 is a member of a family related to the NonO/NonA gene or represents a novel splice variant.

To investigate these possibilities cDNA primers were synthesized to the UG311 sequence in order to perform 5′- and 3′-rapid amplification of cDNA ends (RACE). RACE reactions were performed according to the manufacturer's recommendations except that the internal primer set was subjected to a ramp-up annealing scheme instead of a ramp-down format. The resultant fragments were cloned into pCR2.1 TOPO-TA and were sequenced to confirm overlap between UG311 and the 5′-RACE clones. Two of six RACE fragments had identity in the 150 bp overlap. One other clone had homology only to the primer and the sequence diverged after that point suggesting either spurious priming or the existence of other NonO/NonA family members. These cloned 5′-RACE products extended the UG311 sequence to nearly 1500 bp. Resubmission of this contig. for FASTA to GenBank (data not shown) resulted in the discovery of two nearly identical sequences, nmt55 and p54nrb. The identity of these sequences to UG311-1500 bp was nearly 99% while that of NonO/NonA dropped to 92%. The nmt55 protein was found by screening antibodies generated against the polybasic repeat region of the human estrogen receptor (Sikes et al., Molecular Biology and Biochemistry, pp. 156. Houston:University of Texas Graduate School of Biomedical Sciences, 1993). Western blotting showed no reactivity of these antisera to the estrogen receptor. Instead, there was strong reactivity to an unrelated 55 kDa protein, nmt55/p54nrb is a protein identical to nmt55, found by using antibodies to a yeast mRNA splicing factor to screen a HeLa cDNA expression library (Rajagopal et al., International Journal of Cancer. 62:661-667, 1995). The resultant protein and cDNA bear no resemblance to the yeast splicing factor; however, there was extensive homology to human splicing factor PSF and to drosophila NonA. In HeLa the predominant transcript size was 2.6 kb with a very minor band at 1.9. The open reading frame is virtually identical to nmt55 (Rajagopal et al., International Journal of Cancer. 62:661-667, 1995). This protein was found to be localized to the nucleus and to bind to both single- and double-stranded nucleic acids (Mahana et al., Journal of Immunological Methods. 161:187-192, 1993). Furthermore, nmt55/p54nrb has been demonstrated to facilitate the association of other DNA-binding factors, e.g. topoisomerase I and Ku80, to DNA as well as have a direct role in the transcriptional machinery (Hsieh et al., Cancer Research. 55:190-7, 1995; Southern et al., Journal of Molecular Biology. 98:503-, 1975;Laemmli et al., Nature (London). 227:680-685, 1970). For these reasons nmt55 is thought to be important in either RNA-splicing or DNA repair processes. Additionally, western blotting from normal and cancerous breast samples revealed the loss of nmt55 with the progression of the breast cancer (Sikes et al., Molecular Biology and Biochemistry, pp. 156. Houston:University of Texas Graduate School of Biomedical Sciences, 1993). Interestingly, the open reading frame of nmt55 and p54nrb is found in the first 1600 bases. Thus, if the 5′-RACE of UG311 actually extended to the 5′-end of the mRNA then these genes could be homologous, except for the fact that the longest cDNA for either nmt55 or pnrb54 is only 2.7-2.9 kb or 300-500 bp shorter than the mRNA found in the prostate cancer cell lines.

Therefore, it is of interest to determine the basis for the difference in mRNA lengths of these described related species. Since nmt55 assists other DNA repair enzymes in binding to DNA or may be involved RNA splicing and transcription, it is likely that this protein or other family members represent critical molecules in either cell survival or cell stability. Therefore, cloning and characterization UG311 to determine if it is related to nmt55 or simply another splice variant of a larger mRNA to give the same open reading frame represents a novel and potentially significant step towards understanding a mechanism for prostate cancer progression. The fact that this is lost with breast cancer progression and down regulated in the LNCaP-C4-2 prostate cancer model system implies a functional significance and potential utility for nmt55/UG311 as a prostate cancer marker. For these reasons this study focuses on the cloning and characterization of UG311 to determine the relationship to nmt55 and its role in the biological behavior of prostate cancer. This is the first description, of either a fetal prostate-derived gene or a putative DNA association factor in prostate cancer cell lines with a correlation to progression.

Aim

The cloning and characterization of the full-length cDNA corresponding to the differentially expressed urogenital sinus-derived expressed sequence tag, UG311, from LNCaP or C4-2 lambda gt11 cDNA libraries or by 5′- and 3′-RACE.

Rationale

As described above, it is important to know whether UG311 represents a novel gene closely related to nmt55/nrb54 or merely a splice variant, or processing variant leading to a longer mRNA in prostate. The presence of additional coding sequence would provide clues to tissue-specific RNA splicing or transcription control while additional 3′noncoding sequence may provide information on mRNA stability or potentially tissue specific interactions with other single-stranded nucleic acid binding proteins that associate with these sequences. Therefore, it is necessary to clone the UG311 homolog from prostate cell lines or tissues.

Experimental Approach

Cloning of UG311 cDNA from Lambda gt11 Expression Library

All cell lines and libraries are to be made available from Dr. Leland Chung, Ph.D. LNCaP and C4-2 cell line lambda gt11 phage libraries will be screened for homologous clones to UG311. These clones will be sequenced to determine homology and overlap. Overlapping clones will be reassembled by subcloning with available restriction enzyme sites. These libraries were constructed from poly A+ selected RNA using Invitrogen Custom Services (Invitrogen Corp., San Diego, Calif.) and these libraries have been used previously to clone cDNAs corresponding to differential display PCR fragments (Chen et al JBC 1998). Following long-term storage at −80° C., these libraries will be retitered before screening. Up to one million plaques will be screened for each novel UGS-derived EST. At least 3 plaques will be purified through three rounds of hybridization (Gleave et al., Cancer Research. 52:1598-605, 1992). Hybridization conditions between mouse and human cDNAs have been determined empirically and are performed overnight at 60° C. in 5× standard saline citrate, 10% high molecular weight dextran sulfate, 15% formamide. Preparation of phage DNA will be accomplished by eluting phage from the purified plaques essentially as described (Gleave et al., Cancer Research. 52:1598-605, 1992; Ma et al., Fundamental and Clinical Pharmacology. 10:97-115, 1996). Phage pellets are resuspended in 200 μl Tris-Cl pH 8.0. Polymerase chain reaction (PCR) will be performed on the purified phage to determine the insert size and provide additional template for sequencing after cloning in to TA cloning vectors (Invitrogen Corp., San Diego Calif.).

The use of RACE Reactions to Generate UG311 cDNA

5-prime and 3′-prime race will be performed using the Clontech kit (Clontech, Palo Alto, Calif.). 1 mg of total RNA from the LNCaP cell line will be reverse transcribed. The RNA will be digested and a second strand made. The 5′ and 3′ adapters are then ligated to the double-stranded cDNAs in separate reactions. PCR is then performed with a 5′ adapter specific upstream primer and a gene specific downstream primer as per the manufacturer's recommendations. The PCR products will be evaluated electrophoretically, gel purified, TA cloned as described above and sequenced. Any additional sequence obtained will be subcloned onto the phage-derived cDNA with care taken to exclude RACE primer sequences. Alternatively, RACE reactions can be used to generate the entire homologous cDNA using only overlapping forward and reverse gene specific primers. In this case, the primers would be synthesized from UGS-derived EST's. These RACE products would then be assembled into a contig. and compared to the sequence obtained from the phage inserts. The RACE procedure has been used to acquire an additional 800 bp of the 5′ end of UG311 to yield 1500 bp of sequence to date. Therefore the technique will be repeated on the 3′ end and the overall product compared to the phage insets obtained above.

### EXAMPLE 2

Aim

To screen human prostate cancer specimens by immunohistochemistry (IHC) and in situ hybridization (ISH) for the expression of UG311 (nmt54) to determine if a significant correlation of UG311 expression to stage and grade, prognosis or patient survival exists.

Rationale

Antibodies to nmt55/nrb54 can be generated using routine methods well known in the art. Since nmt55 has virtual identity over most of the putative open reading frame with UG311-1500, its staining pattern should reflect the pattern that would be observed for UG311. Also, for a marker to be useful it must be able to distinguish between either the presence or absence of disease or be able to determine prognosis. Markers with such properties allow for patients to be stratified for either more or less aggressive therapeutic options. Therefore, this study seeks to determine if such a correlation exists for nmt55/UG311 in human prostate cancer specimens.

Experimental Approach

A cohort of 72 prostate cancer specimens will be examined by ISH and IHC. IHC was performed on both fresh frozen and paraffin embedded specimens (Sikes and Chung, Cancer Res. 1992). IHC will be done by the indirect calorimetric detection using DAB as the chromagen donor to a horse-radish peroxidase conjugated secondary antibody. Additionally, Dr. Robert Moreland has supplied detailed protocols for the nmt55 antibodies that include IHC, western blotting and immunoprecipitation (Sikes et al., Molecular Biology and Biochemistry, pp. 156. Houston:University of Texas Graduate School of Biomedical Sciences, 1993). The degree of staining will be scored and the tabulated data will be analyzed for significance and correlation to survival and staging.

Since some tissues will not react to IHC and others not to ISH, both will be done to fully cover the expression of nmt55 in the cohort. Furthermore, ISH provides complementary data on the localization of the mRNA for comparison to the localization of the protein. Colocalization is anticipated. Briefly, the protocol for non-radioactive ISH on paraffin embedded tissue sections is as follows: In situ hybridization will be performed using 30 ng of probe for each slide including antisense probes, sense probes as negative controls, and β-actin probes as positive controls as previously described (Gotoh et al., The Journal of Urology. In Press:, 1997; Akiyama et al., Fibronectin and integrins in invasion and metastasis., Cancer metastasis and reviews. 14:173-189, 1995). The tissue distribution of UG311 and β-actin will be determined by immunohistochemical staining methods developed in our laboratory. The intensity and the distribution of mRNA staining will be scored as follows: ++, diffuse localization in >25% of cells; +, focal localization in <25% of cells; −, negative.

If significant differences in UG311 cDNA and the nmt55 open reading frame are found, then the UG311 cDNA will be cloned into bacterial expression vector for amplification and purification. Purified UGS derived gene fusion-proteins will be used as an immunogen for the generation of polyclonal antibodies. Antibodies will be tested for reactivity under reducing and nonreducing conditions as well as paraffin-embedded and frozen tissue sections.

The purpose of this study is to generate several high quality antibodies against the UG311 gene product to facilitate the study of its biology and biochemistry in prostate cancer. Antibodies are desired that will react positively to the UG311 gene product in immunohistochemistry (cell lines and paraffin sections) western blots (reduced and non-reducing conditions) and immunoprecipitation. Since peptide-derived antibodies frequently fail to work well for all biochemical applications, the use of peptides to generate antibodies will be an alternative secondary option. First, fusion proteins will be generated from the UG311 ORF for the production of antibodies.

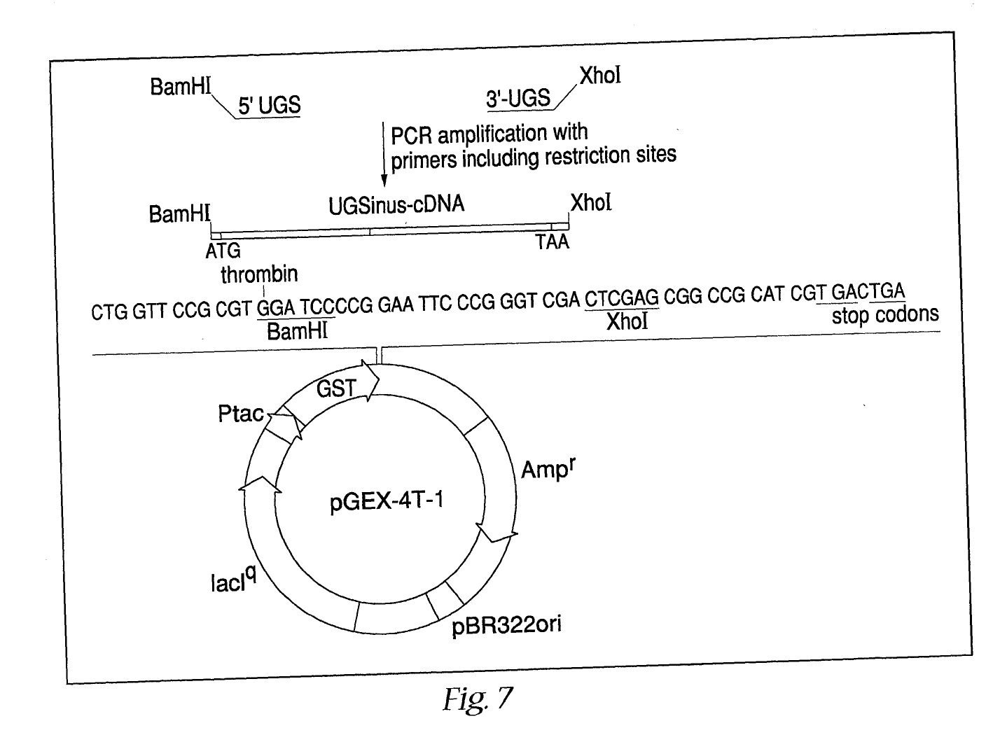

Bacterial expression and purification of many proteins or protein fragments has allowed for the generation of antibodies to a wide variety of proteins including difficult, i.e., poorly immunogenic or highly conserved proteins (Ziober et al., Seminars in Cancer Biology. 7:119-128, 1996). This strategy will be employed to generate large amount of purified UG311 gene product. The UG311 ORF will be cloned into bacterial expression vector, pGEX-4T (Pharmacia Biotech, Piscataway N.J.) (FIG. 7). This plasmid generates a glutathione S-transferase (GST) fusion protein with the protein of interest when expressed in appropriate bacterial hosts. The GST portion allows for both the facilitated monitoring of fusion protein expression using a solution-based calorimetric assay in crude cell lysates as well as the ease of protein purification using a glutathione column. Polymerase chain reaction will be used to amplify the UG311 ORF incorporating appropriate in-frame restriction endonuclease sites for directional subcloning. This approach allows one to bypass any potential 5′-UTR that may be present and directly clone the UG311 coding sequences in-frame behind the GST fusion tag. UGS-derived gene products will be purified from bacterial hosts according to the manufacturer's recommendations. For pGEX4T expressed protein, purification will be accomplished by binding to a glutathione column followed by thrombin cleavage to remove the GST fusion protein. Thrombin will be removed by passing the eluate over a benzamidine sepharose column. Rapid preliminary detection of GST-fusion constructs can be ascertained by using a GST-detection kit (Pharmacia, Piscataway N.J.). Protein yield will be estimated by Bradford and purity followed by SDS-PAGE in 12.5 to 15% acrylamide gels in both systems.

EXAMPLE 3

Aim

To assess the possible direct and indirect biologic functions of the UG311/nmt55 in prostate cancer progression.

Rationale

Since nmt55 has been shown to be lost in breast cancer progression and is associated with estrogen receptor negativity, a major prognostic factor for breast cancer, then it follows that the expression of nmt55/UG311 should be manipulated in prostate cancer cell lines to directly test whether or not the loss/overexpression of nmt55UG311 protein can modulate the aggressiveness of prostate cancer. Levels of UG311 gene expression in the LNCaP model of human prostate cancer progression will be manipulated using an inducible mammalian expression system (TET-on) in conjunction with protein tagging by using a FLAG epitope. It will be determined if the overexpression of these UGS-derived genes may decrease prostate cancer growth, invasiveness and/or metastatic potential. Conversely, suppressing the levels of UG311 gene expression by antisense technology may confer increased tumorigenicity and metastatic potential.

Since, the LNCaP/C4-2 model closely mimics the natural progression of human prostate cancer from non-metastasizing, androgen-dependent cells (LNCaP) that are gradually transformed in vivo into aggressively-metastasizing, androgen-independent cells (C4-2) this model represents an ideal system to test UG311 function by reducing the protein levels in LNCaP and by re-expression of the protein in C4-2 cells. Never before have fetal urogenital sinus-derived genes been associated with the malignant potential of prostate cancer. Further characterization of this gene and others should clarifying the role of embryonic influences on prostate carcinogenesis, as well as identify and develop novel prognostic markers and potential targets for gene therapy and other therapeutic modalities for treating human prostate cancer.

Experimental Approach

Example 2 presented above already examines whether or not there is a similar loss of nmt55/UG311 expression between human breast and prostate cancer tissues; this study will manipulate the gene product levels in a human prostate cancer progression model by using sense and antisense gene expression techniques in an inducible vector system to directly test the effects of UG311 protein levels on prostate cancer cell behavior. In order to assess the possible direct and indirect biologic functions of these genes in prostate cancer progression, the levels of UG311 expression in the experimental LNCaP model of human prostate cancer progression will be manipulated. This study will determine if overexpression of these genes may arrest prostate cancer growth and decrease its invasiveness and metastatic potential. Conversely, antisense constructs will be used to lower the steady-state levels of UG311 in the hope that reduced expression will increase invasive and metastatic potential.

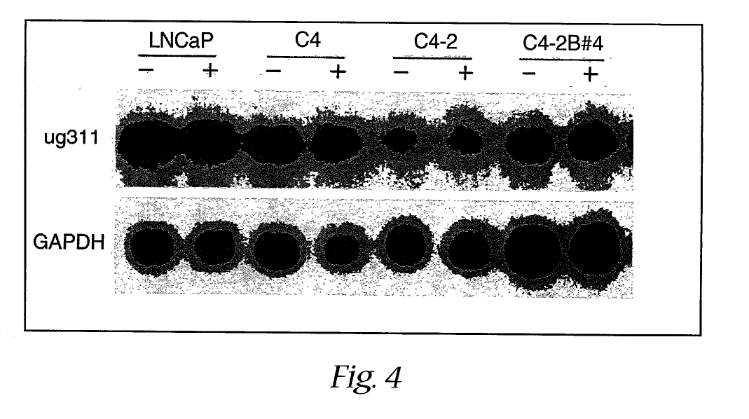

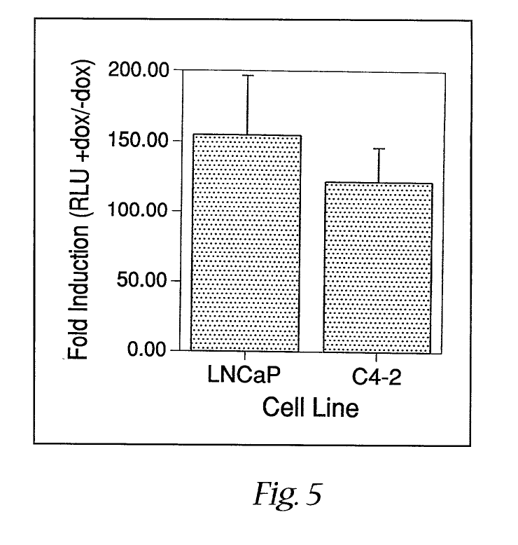

Previously, several cDNA constructs in both rat and human prostate cancer cell lines have been cloned, transfected and overexpressed (Sikes and Chung, Cancer Res. 1992) (Levine et al., EXS. 74:157-179, 1995; Nagle et al.,, Journal of Cellular Biochemistry, Supplement. 19:232-237, 1994; Umbas et al., Cancer Research. 52:5104-9,1992). Overexpression of sense cDNAs has been employed with some success to evaluate gene product function in prostate cell lines (Levine et al., EXS. 74:157-179, 1995; Umbas et al.,Cancer Research. 52:5104-9, 1992; Freeman et al., Cancer Research. 51:1910-6, 1991). Likewise, antisense strategies employing full-length cDNA constructs have proven effective for the EGF receptor in colon carcinoma and C-CAM in prostate epithelia (Bussemakers et al., Cancer Research. 52:2916-22, 1992; Chung et al., Journal of Cellular Biochemistry—Supplement. 16H:99-105, 1992). Tet-responsive clones for LNCaP and C4-2 have already been generated using the TET-on system from Clontech and have been shown to induce the level of luciferase reporter gene expression by more than 125 fold (FIG. 4). Sense and antisense constructs of UG311 fused to FLAG-tag element will be amplified by PCR and subcloned into the VP-16 responsive vector for Doxycycline (TET) induction. Protein levels can be followed by both anti-FLAG and nmt55 antibodies. Sense and antisense riboprobes will follow the levels of RNA produced by RNA blot. One correct sense and one correct antisense clone will be expanded, purified by CsCl banding and sequenced by dideoxy chain termination using the ALF/express system from Pharmacia and Cy5 amidite fluorescent primers to confirm sequence fidelity and orientation.

Western blots will be performed as described in Sikes and Chung (Nagle et al., Journal of Cellular Biochemistry, Supplement. 19:232-237,1994) in the presence of protease inhibitors to determine the levels of UG311 gene products being expressed in the transfected cell lines. Enhanced chemiluminescent (ECL) detection of the UG311 protein will be performed according to the manufacturer's recommendations (Amersham, Arlington Heights, Ill.).

One of the sense and antisense UG311 tranfected clones, selected as described above, will be assessed for changes in their tumorigenic behavior by determining both anchorage independent growth, their cell migration/invasive potential in Matrigel® and tumor development in vivo as determined by subcutaneous (s.c.) injections into athymic male mouse hosts. Anchorage independent growth of sense and antisense clones will be assessed as described previously (Wu et al., The International Journal of Cancer. Submitted October 1997, 1997) (Inventors: please confirm the citation for this reference). Either 1000 or 5000 cells/ 6-well chamber will be mixed with an equal volume (1 ml) of low melting point agarose in distilled H₂O. Cells will be monitored for 6-8 weeks at which time colonies ≧0.4 mm diameter will be counted using a dissection microscope. Modified Boyden chamber assays will be used to assess tumor cell migration and invasiveness. The results of invasion assays will be correlated to the steady-state levels of UG311 protein expressed in the clones.

For tumorigenicity in vivo (Thalmann et al., Cancer Research. 54:2577-2581,1994; Wu et al., The International Journal of Cancer. Submitted October 1997, 1997), transfected cells prepared as above will be resuspended in T-medium/5% FBS at the appropriate cell number and injected using a graduated insulin syringe. UG311-Flag-Tet-on sense and antisense transfected LNCaP and C4-2 cell clones will be injected into intact nude mice at 4×10⁶ cells per 100 μl s.c. Tumors will be allowed to develop for 6 weeks or until the tumor mass has reached 1.5 cc at which time the animals will be euthanized. Tissues will be harvested, fixed in neutral-buffered formalin for less than 16 hrs, and sent to the pathology department for paraffin embedding and sectioning. Slides will be routinely stained with hematoxylin and eosin and read by the pathologist to determine the presence of cancer cells. Sections will be stained additionally as in Sikes and Chung (1992)(Nagle et al.,, Journal of Cellular Biochemistry, Supplement. 19:232-237, 1994) or Gleave et al (1992) (Liotta et al., Annual Review of Biochemistry. 55:1037-1057, 1986) for Ki67, PSA and tunel to monitor the extent of prostate growth, differentiation and apoptosis, respectively. These will be correlated to transfected cell status, tumor growth and invasive potential.

There are to be no expected difficulties in making the cDNA gal constructs. Antisense technology, however, can be unpredictable with variable impact on the expression of the sense RNA to any gene of interest. Alternatives include: 1) antisense constructs directed at only 5′UTR and transcription initiation site (Mackay et al., Invasion Metastasis. 12:168-184, 1992). 2) design a Ribozyme directed at the UGS-derived mRNA or 3) design antisense oligonucleotides to the 5-prime end or transcription initiation site to knock-out UGS-derived gene expression.

### EXAMPLE 4

While the prostate cancer cell LNCaP/C4-2 model described above in Example 1 closely mimics the natural progression of human prostate cancer from non-metastasizing, androgen-dependent cells (LNCaP) that are gradually transformed in vivo into aggressively-metastasizing, androgen-independent cells (C4-2), this model represents only one of the model systems used herein to assay for UGS-derived fetal prostate gene function by reducing the protein levels in LNCaP and by re-expression of the protein in C4-2 cells. Other cell systems, however, may also be used in the present invention to assay for UGS-derived fetal prostate gene function, including, for example, without limitation, normal prostate tissue in conjunction with prostate cancer tissue, and early prostate cancer tissue in conjunction with metastatic prostate cancer tissue. The biological sample to be analyzed in these alternative models may be any tissue or fluid in which prostate cancer cells might be present. Various embodiments include bone marrow aspirate, bone marrow biopsy, lymph node aspirate, lymph node biopsy, spleen tissue, fine needle aspirate, skin biopsy or organ tissue biopsy.

All developmental switches have a role in prostate development and/or diseases of the prostate, including, without limitation, prostatitis, and benign and malignant growth of the prostate gland. Such developmental switches include proteins encoded by messenger RNAs including, for example, without limitation, certain messenger RNAs listed in Tables 1-5. In particular, such developmental switches include those mRNAs which encode proteins including, for example, without limitation: ugs186oft which encodes mus musculus (mouse). protein kinase clk (ec 2.7.1.-) (483aa) or related proteins; ugs 160 which encodes homo sapiens (human). kinesin-like protein eg5.10/1996 (1057 aa) or related proteins; ug381 which encodes homo sapiens (human) elongation factor 1-beta (ef-1-beta)(224 aa) or related proteins; ugs101 which encodes mus musculus (mouse) retrovirus-related pol polyprotei (1300 aa) or related proteins; ug485ors which encodes homo sapiens (human) putative rna-binding protein rnpl (157aa) or related proteins; ug356 which encodes rattus norvegicus (rat), and mus musculus (mouse) heat shock cognate 71 kDa (646 aa) or related proteins; ug108rcon which encodes escherichia coli tetracycline repressor protein class (216 aa) or related proteins; ugs045 which encodes Rattus norvegicus Smad4 protein Smad4 mRNA, complete cds. 4/98 (3041 nt) or related proteins; ug048 which encodes Human DNA sequence from PAC 434P1 on chromosome 22 Contains (45548 nt)or related proteins; ugs225 which encodes Mus musculus chromosome 19, clone D19-96, B7, complete sequence. (769037 nt)or related proteins; ug156rcon which encodes Homo sapiens protein associated which encodes Myc mRNA, complete cds. 8/98 (14807 nt)or related proteins; ug157rcon which encodes Homo sapiens ALR mRNA, complete cds. 9/97 (trx-G paralogue, trithorax gene complex, homeotic) (15789 nt)or related proteins; ug 192rcon which encodes Human FUSE binding protein mRNA, complete cds. 5/94 (2325 nt).

In addition, such developmental switches include those listed in Table 1, wherein the results of the library analysis of 728 cDNA UGS-derived ESTs are presented using the Swissprot database, including, for example, without limitation: ug517 which encodes mus musculus (mouse). k-glypican precursor. 10/1996 (557 aa)or related proteins; ugs016 which encodes mus musculus (mouse). bone proteoglycanII precursor (p(354 aa)or related proteins; ua2h6f which encodes mus musculus (mouse). insulin-like growth factor bindin (305 aa)or related proteins; ug 130 which encodes mus musculus (mouse). insulin-like growth factor bindin (271aa) or related proteins; ua1a2 which encodes Homo sapiens (human) son protein (son3). DNA binding protein w/ mos and myc homology 11/1995 (1523 aa)or related proteins; ug271 which encodes mus musculus (mouse). carg-binding factor-a (cbf-a). 11(285aa)or related proteins; ug277t which encodes ambystoma mexicanum (axolotl). homeotic protein hox-a13 (107 aa)or related proteins; ug367 which encodes mus musculus (mouse). embryonic tea domain-containing factor (445 aa)or related proteins; ug486 which encodes rattus norvegicus (rat). lim protein clp36. (contains homeodomain of lin-11) 10/1996 (327 aa)or related proteins; ug293 which encodes Homo sapiens (human). ptb-associated splicing factor ps (707 aa)or related proteins; ug485ors which encodes Homo sapiens (human). putative ma-binding protein rnpl (157aa)or related proteins; ug101rcon which encodes mus musculus (mouse). dipeptidyl peptidase iv (ec 3.4.1(760aa)or related proteins; ug211 which encodes mus musculus (mouse). matrix metalloproteinase-14 precu (582 aa)or related proteins; ug335 which encodes rattus norvegicus (rat). neprilysin (ec 3.4.24.11)(neutra (749 aa)or related proteins; ugs044 which encodes mus musculus (mouse). tlm protein (tlm oncogene). 12/199 (317 aa).

In particular, such developmental switches additionally include those in listed in Table 2 wherein the results of the library analysis of 728 cDNA UGS-derived ESTs are presented using the GENPEPT translated protein database (rel 102.0), including, for example, without limitation: ug135 which encodes breast adenocarcinoma metastasis-associated gene (contains SH3 domains) Homo sapiens (715aa).

In particular, such developmental switches additionally include those listed in Table 3, wherein the results of the library analysis of 728 cDNA UGS-derived ESTs using the primate rodent GB 103 database, including, for example, without limitation: ugs 186s which encodes Mus musculus cdc2/CDC28-like protein kinase 4 (Clk4) mRNA, comple (1549 nt)or related proteins; ug206 which encodes Rat mRNA for short type PB-cadherin, complete cds. 7/96 (4153 nt)or related proteins; ug392 which encodes Mus musculus vascular adhesion protein-I gene, complete cds.9/98 (14357 nt)or related proteins; ug142** which encodes Mus musculus tumor susceptibility protein 101 (tsg101) gene, comp (33613 nt)or related proteins; ug219** which encodes Mus musculus tumor susceptibility protein 101 (tsg101) gene, comp (33613 nt)or related proteins; ugs216 which encodes Mus musculus retinoblastoma-related protein p130 mRNA (4013 nt)or related proteins; ug414 which encodes Murine gene for interleukin 5 (eosinophil differentiation fac (6727 nt)or related proteins; ug159 which encodes Mus musculus WW domain binding protein 5 mRNA, partial cds. (proline-rich, sh3domain interactive protein) involved in regulation of transcription in development of kidney and limbs. Homologue of Drosophila enabled. (647 nt)or related proteins; ug422 which encodes Mus musculus timeless homolog mRNA, complete cds. 11/98 (4438 nt) 7. le-47 (Mammalian Circadian Autoregulatory Loop: A Timeless Ortholog and mPER1 Interact and Negatively Regulate CLOCK-BMAL 1-Induced Transcription) ugs045 which encodes Rattus norvegicus Smad4 protein (Smad4) mRNA, complete cds. 4/98 (3041 nt)or related proteins; ugs 192 which encodes Homo sapiens protein associated which encodes Myc mRNA, complete cds. 8/98 (14807 nt)or related proteins; ugs213 which encodes Mus musculus dishevelled-3 (Dvl-3) mRNA, complete cds. 6/96 (2498 nt)or related proteins; ugs218 which encodes Human Krueppel-related zinc finger protein (H-plk) mRNA, com (2873 nt)or related proteins; ug281 which encodes Human mitosin mRNA (mitotic progression factor), complete cds. 12/95 (10211 nt)or related proteins; ugs234 which encodes mus musculus high mobility group protein homolog HMG4 (Hmg4) mRNA (1502 nt)or related proteins; ug494 which encodes Human alternative splicing factor mRNA, complete cds. 9/91 (1717 nt)or related proteins; ug088rcon which encodes mus musculus matrix metalloproteinase-14 (Mmp14), exons 9 (1242 nt)or related proteins; ug179rcon which encodes mus musculus ATP-dependent metalloprotease FtsHl mRNA, complete clone (2654 nt) or related proteins; ug380 which encodes mus musculus male-enhanced antigen (Mea) mRNA (human chromo 6p21.1-21.3), complete cds. (841 nt).

In particular, such developmental switches additionally include those in listed in Table 4 wherein the results of the library analysis of 728 cDNA UGS-derived ESTs are presented using the GenBank database, including, for example, without limitation: ug031 con which encodes mus musculus vascular adhesion protein-1 gene. complete cds. 9/98 (14357 nt)or related proteins; ug059 which encodes Homo sapiens gene for osteonidogen, intron 9. 3/98 (9085 nt)or related proteins; ug039rcon which encodes mus musculus 9ORF binding protein 19BP-1 mRNA, Binding of Human Virus Oncoproteins to hDlg/SAP97, a Mammalian Homolog of the Drosophila Discs large Tumor Suppressor protein (2703 nt)or related proteins; ug051rcon which encodes Mouse mRNA for prothymosin alpha. 6/91 (1191 nt)or related proteins; ug033con which encodes M.musculus TSC-22 mRNA. Isolation of a gene encoding a putative leucine zipper structure that is induced by transforming growth factor beta 1 and other growth factors. 12/93 (1706 nt)or related proteins; ug092ft which encodes Gallus gallus single-strand DNA-binding protein.csdp SSDP (sequence-specific single-stranded DNA-binding protein), mRNA,(1211 nt)or related proteins; ug092ors which encodes fb33f07.yl Zebrafish WashU MPIMG EST Danio rerio cDNA 5′ similar to Gallus gallus single-strand DNA-binding protein. csdp SSDP (sequence-specific single-stranded DNA- binding protein), mRNA (396 nt).

This comprehensive approach and evaluation as listed above in Examples 1-4 permits the discovery of novel genes and gene products, from among the UGS-derived EST cDNA clone designations provided, inter alia, in FIG. 1, FIG. 9, and as presented in Tables 6 and 7, as well as the identification of an array of genes and gene products (whether novel or known) involved in novel pathways that play a major role in prostate disease pathology. Thus, the invention allows one to define targets useful for diagnosis, monitoring, rational drug screening and design, and/or other therapeutic intervention for prostatic disease processes, including but not limited to, prostatitis, and benign and malignant growth of the prostate gland.

All publications, patents and patent applications mentioned in this specification are herein incorporated by reference in to the specification to the same extent as if each individual publication, patent or patent application was specifically and individually indicated to be incorporated herein by reference.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention.

What is claimed is:

1. An isolated polynucleotide comprising a nucleotide sequence containing the urogenital sinus-derived expressed sequence tag comprising ug092, ug093, ug096, ug101, ug102, ug106, ug120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194.

2. An isolated polynucleotide comprising a nucleotide sequence having a urogenital sinus-derived expressed sequence tag sequence at least 95% identical to a sequence comprising ug092, ug093, ug096, ug101, ug102, ug106, ug120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194.

3. An isolated polynucleotide encoding a polypeptide wherein, except for at least one conservative amino acid substitution, said polypeptide has an amino acid sequence that is identical to a urogenital sinus-derived express sequence tag comprising ug092, ug093, ug096, ug101, ug102, ug106, ug120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194.

4. An isolated polynucleotide comprising a nucleotide sequence containing the urogenital sinus-derived expressed sequence tag comprising ug311.

5. An isolated polynucleotide of claim 1 which is DNA.

6. The isolated polynucleotide of claim 1 which is cDNA.

7. The isolated polynucleotide of claim 1 which is genomic DNA.

8. The isolated polynucleotide of claim 1 which is RNA.

9. The isolated polynucleotide of claim 1 which further comprises a detectable label.

10. A polynucleotide vector containing the polynucleotide of claim 1.

11. A polynucleotide expression vector containing the polynucleotide of claim 1 in operative association with a nucleotide regulatory element that controls expression of the polynucleotide in a host cell.

12. A cultured genetically engineered host cell containing the polynucleotide of claim 1.

13. A cultured genetically engineered host cell containing the polynucleotide of claim 1 in operative association with a nucleotide regulatory element that controls expression of the polynucleotide in the host cell.

14. The genetically engineered host cell of claim 13 which is prokaryotic.

15. The genetically engineered host cell of claim 13 which is eukaryotic.

16. A method of producing a polypeptide urogenital sinus-derived gene product, comprising the steps of:
(a) growing the genetically engineered host cell of claim 14 in a culture; and
(b) collecting the polypeptide gene product from the culture.

17. A method of producing a polypeptide urogenital sinus-derived gene product, comprising the steps of:
(a) growing the genetically engineered host cell of claim 15 in a culture; and
(b) collecting the polypeptide gene product from the culture.

18. An isolated polypeptide comprising the amino acid sequence encoded by the nucleotide sequence containing the urogenital sinus-derived expressed sequence tag comprising ug092, ug093, ug096, ug101, ug102, ug106, ug120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194.

19. A fusion protein comprising the polypeptide of claim 16 operatively associated with a heterologous polypeptide.

20. An isolated polypeptide comprising a polypeptide having an amino acid sequence at least 95% identical to the amino acid sequence encoded by the nucleotide sequence containing the urogenital sinus-derived expressed sequence tag comprising ug092, ug093, ug096, ug101, ug102, ug106, ug120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194.

21. A fusion protein comprising the polypeptide of claim 18 or 20 operatively associated with a heterologous polypeptide.

22. An isolated polypeptide comprising the amino acid sequence encoded by the nucleotide sequence containing the urogenital sinus-derived expressed sequence tag comprising ug311.

23. A pharmaceutical composition comprising
(a) An isolated polypeptide comprising the amino acid sequence encoded by the nucleotide sequence containing the urogenital sinus-derived expressed sequence tag comprising ug092, ug093, ug096, ug101, ug102, ug106, ug120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194;
(b) pharmaceutically acceptable salts thereof; and a pharmaceutically acceptable carrier.

24. A pharmaceutical composition comprising
(a) An isolated polypeptide comprising a polypeptide having an amino acid sequence at least 95% identical to the amino acid sequence encoded by the nucleotide sequence containing the urogenital sinus-derived expressed sequence tag comprising ug092, ug093, ug096, ug101, ug102, ug106, ug120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194;
(b) pharmaceutically acceptable salts thereof; and a pharmaceutically acceptable carrier.

25. A method for diagnosing prostate disease, comprising assaying, in a patient sample, the expression of a polynucleotide containing the urogenital sinus-derived expressed sequence tag comprising ug092, ug093, ug096, ug101, ug102, ug106, ug120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194.

26. The method of claim 25 in which the expression of the polynucleotide is up-regulated.

27. The method of claim 25 in which the expression of the polynucleotide is down-regulated.

28. A method of monitoring the efficacy of a compound in clinical trials for the treatment of prostate disease, comprising assaying, in a patient sample, the expression of a polynucleotide containing the urogenital sinus-derived expressed sequence tag comprising ug092, ug093, ug096, ug101, ug102, ug106, ug120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194.

29. The method of claim 28 in which the expression of the polynucleotide is up-regulated.

30. The method of claim 28 in which the expression of the polynucleotide is down-regulated.

31. The method of claim 25 or 28 in which differential expression of the polynucleotide is assayed by:
(a) obtaining a sample of cells from a patient;
(b) assaying the expression of the polynucleotide in the sample of cells; and
(c) comparing the expression level of the polynucleotide in the patient sample to the expression level of the polynucleotide in a control sample of cells, in which a difference in the expression level of the polynucleotide in the patient sample and the control indicates differential expression of the polynucleotide.

32. A method for diagnosing prostate disease, comprising determining, in a patient sample, the presence of a mutation in a gene containing the urogenital sinus-derived expressed sequence tag comprising ug092, ug093, ug096, ug101, ug102, ug106, ug120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194.

33. The method of claim 32 in which the mutation of the gene is assayed by:
(a) obtaining a sample of cells from the patient;
(b) analyzing the structure of the gene in genomic DNA obtained from the sample of cells; and
(c) comparing the structure of the gene in the patient sample to the structure of the gene in a control sample of cells, in which a difference in the structure of the gene in the patient sample and the control indicates a mutation in the gene in the patient.

34. A method for identifying a substance for treating prostate disease comprising assaying the ability of a test substance to modulate the expression of a gene containing the urogenital sinus-derived expressed sequence tag comprising ug092, ug093, ug096, ug101, ug102, ug106, ug120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194.

35. The method of claim 34 in which the prostate disease is prostatitis, benign or malignant growth of the prostate gland.

36. The method of claim 34 in which the modulation of the expression of said gene is assayed by:
(a) exposing a sample of cells to a test substance;
(b) assaying the expression of said gene in the sample of cells; and
(c) comparing the expression level of the gene in the sample exposed to the substance to the expression level of the gene in a control sample of cells, in which a difference between the expression level of the gene in the sample exposed to the substance and the control indicates the modulation of expression of the gene.

37. The method of claim 34 in which the gene is down-regulated by the test substance.

38. The method of claim 36 in which the substance is an oligonucleotide complementary to the 5′ region of the gene and blocks transcription via triple helix formation.

39. The method of claim 38 in which the substance is an antisense or ribozyme molecule that blocks translation of the gene.

40. The method of claim 34 in which the gene is up-regulated by the test substance.

41. The method of claim 34 in which the substance is a small organic or inorganic molecule that modulates the activity of the protein product by binding to the protein product.

42. The method of claim 34 in which the substance is an antibody that modulates the activity of the protein product by binding to the protein product.

43. An assay for identifying a substance that binds to the protein encoded by a gene comprising:
(a) contacting a protein or peptide containing an amino acid sequence corresponding to the binding site of the protein encoded by a gene containing a urogenital sinus-derived expressed sequence tag comprising ug092, ug093, ug096, ug101, ug102, ug106, ug120, ug254, ug291, ug307, ug308, ug311, ug317, ug320, ug334, ug335, ug353, ug354, ug357, ug440, ug441, ug482, ug484, ug485, ug491, ug493, ug494, ug503, ug505, ug506, ugs148, ugs186, and ugs194, with a test substance, under conditions and for a time sufficient to permit binding and formation of a complex between the protein or peptide and the test substance, and
(b) detecting the formation of a complex, in which the ability of the test substance to bind to the protein is indicated by the presence of the test substance in the complex.
